(19) United States
(12) Patent Application Publication
(10) Pub. No.: US 2023/0089867 A1
(43) Pub. Date: Mar. 23, 2023

(54) CYCLIC PENTAMER COMPOUNDS AS PROPROTEIN CONVERTASE SUBTILISIN/KEXIN TYPE 9 (PCSK9) INHIBITORS FOR THE TREATMENT OF METABOLIC DISORDER

(71) Applicant: Novartis AG, Basel (CH)

(72) Inventors: Christopher BECKER, Stow, MA (US); Brian Addison DECHRISTOPHER, Belmont, MA (US); Alec Nathanson FLYER, Cambridge, MA (US); Adam FRIEDMAN, Cambridge, MA (US); Christian GAMPE, Watertown, MA (US); Andrei Alexandrovich GOLOSOV, Cambridge, MA (US); Philipp GROSCHE, Riehenstrasse (DE); Tyler HARRISON, Arlington, MA (US); Qian LIU, Belmont, MA (US); Eugene Yuejin LIU, I, Lexington, MA (US); Justin Yik Ching MAO, North Reading, MA (US); Lauren Gilchrist MONOVICH, Belmont, MA (US); Tajesh Jayprakash PATEL, Westford, MA (US); Carina Cristina SANCHEZ, Somerville, MA (US); Liansheng SU, Winchester, MA (US); Lihua YANG, Westford, MA (US); Rui ZHENG, Lexington, MA (US)

(21) Appl. No.: 17/296,895

(22) PCT Filed: Nov. 26, 2019
(86) PCT No.: PCT/IB2019/060200

§ 371 Date: May 25, 2021

Related U.S. Application Data
(60) Provisional application No. 62/772,029, filed on Nov. 27, 2018.
(60) Provisional application No. 62/925,108, filed on Oct. 23, 2019.

(51) Int. Cl. C07K 7/56

(52) U.S. Cl. CPC C07K 7/56; A61K 38/00

(57) ABSTRACT

The disclosure relates to inhibitors of PCSK9 useful in the treatment of cholesterol lipid metabolism, and other diseases in which PCSK9 plays a role, having the Formula (I):

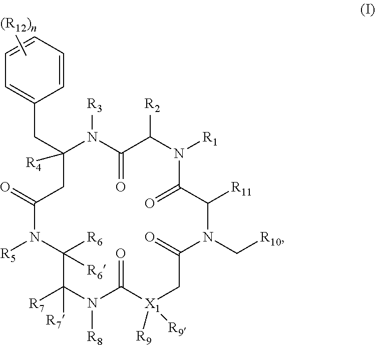

or a pharmaceutically acceptable salt, hydrate, solvate, prodrug, stereoisomer, N-oxide, or tautomer thereof, wherein X₁, R₁, R₂, R₃, R₄, R₅, R₆, R_{6′}, R₇, R_{7′}, R₈, R₉, R_{9′}, R₁₀, R₁₁, R₁₂, and n are described herein.

### RELATED APPLICATIONS

This application is a U.S. National Phase filing of International Application Serial No. PCT/IB2019/060200, filed Nov. 26, 2019, and claims the benefit of and priority to U.S. Provisional Application No. 62/772,029, filed Nov. 27, 2018, and U.S. Provisional Application No. 62/925,108, filed Oct. 23, 2019, the entire contents of each of which are incorporated herein by reference in their entireties.

### FIELD OF DISCLOSURE

The present disclosure is directed to modulators of proprotein convertase subtilisin/kexin type 9 (PCSK9) useful in the treatment of diseases or disorders associated with PCSK9 protease. Specifically, the disclosure is concerned with compounds and compositions, which inhibit PCSK9, methods of treating diseases or disorders associated with PCSK9, and methods of synthesis of these compounds.

### BACKGROUND OF THE DISCLOSURE

Proprotein convertase subtilisin/kexin type 9 (PCSK9) is a member of the secretory subtilase, subtilisin serine protease family, and is expressed in many tissues and cell types. The PCSK9 protein contains a signal sequence, a prodomain, a catalytic domain containing a conserved triad of residues (D186, H226 and S386), and a C-terminal domain and is synthesized as a soluble 74-kDa precursor that undergoes autocatalytic cleavage in the endoplasmic reticulum. The autocatalytic activity has been shown to be required for secretion.

PCSK9 has pronounced effects on plasma low density lipoprotein cholesterol (LDL-C) levels via its modulation of hepatic low density lipoprotein receptors (LDLR), the main route by which cholesterol is removed from the circulation. PCSK9 binds the LDLR and directs it to lysosomal degradation, thereby increasing plasma LDL-C levels and, in turn, coronary heart disease (CHD) risk. (Maxwell K. N., Proc. Natl. Acad. Sci., 101, 2004, 7100-7105; Park, S. W., J. Biol. Chem. 279, 2004, 50630-50638; Lagace T. A., et. al. J. Clin. Invest. 2006, 116(11):2995-3005). Overexpression of mouse or human PCSK9 in mice has been shown to elevate total and LDL-C levels and dramatically reduce hepatic LDLR protein, without an observed effect on the levels of mRNA, SREBP, or SREBP protein nuclear to cytoplasmic ratio. (Maxwell K. N., Proc. Natl. Acad. Sci. 101, 2004, 7100-7105). Moreover, mutations in PCSK9 that cause loss of PCSK9 function in mouse models have also been shown to lower total and LDL-C levels. (Cohen, J. C., et al., N. Engl. J. Med., 354, 2006, 1264-1272). Thus, the results indicate that modulation of PCSK9 results in a reduction of LDLR protein levels.

Gene deletion of PCSK9 has also been conducted in mice. PCSK9 knockout mice show an approximate 50% reduction in plasma cholesterol levels and enhanced sensitivity to statins in reducing plasma cholesterol (Rashid, S., et al., Proc. Natl. Acad. Sci., 2005, 102:5374-5379). Human genetic data strongly support the role of PCSK9 in LDL homeostasis. The link between PCSK9 and plasma LDL-C levels was first established by the discovery of PCSK9 missense mutations in patients with an autosomal dominant form of familial hypercholesterolemia (Abifadel, M., et al., Nature, 2003, 34:154-6). Patients carrying PCSK9 gain-of-function alleles have increased plasma LDL-C levels and premature CHD, whereas those with PCSK9 loss-of-function alleles have markedly reduced plasma LDL-C and are protected from CHD.

PCSK9 also plays a role in Lipoprotein (a) (Lp(a)) metabolism. Lp(a) is a proatherogenic lipoprotein comprised of an LDL particle covalently linked to apoLp(a). Human genetic studies indicate that Lp(a) is causally associated with CHD risk. PCSK9 therapeutic antibodies have been shown to significantly reduce Lp(a) levels in patients with hypercholesterolemia. (Desai, N. R., et. al., Circulation. 2013, 128(9):962-969; Lambert, G., et. al., Clin. Sci., 2017, 131, 261-268). Patients receiving statin therapy treated with a monoclonal antibody against PCSK9 have shown up to 32% reduction in Lp(a) levels compared to placebo. (Desai N. R., et. al., Circulation. 2013, 128(9):962-969).

In addition to having cardiovascular effects, PCSK9 plays an important role in sepsis, a life-threatening condition caused by a body's response to infection. Overexpression of PSCK9 in septic mice has been shown to aggravate sepsis by increasing inflammation, while inhibition of PCSK9 has been shown to reduce mortality. (Dwivedi, D. J., et al., Shock, 2016, 46(6), 672-680). Moreover, flow cytometry studies in human HepG2 cells have shown that PCSK9 negatively regulates gram-negative lipopolysaccharide (LPS) uptake by hepatocytes through the regulation of the LDLR-mediated bacterial lipid uptake of lipoteichoic acid (LTA) and LPS through an LDL-dependent mechanism. (Grin, P. M., et al., Nature, 2018, 8(1):10496) Thus, inhibition of PCSK9 has the potential to treat sepsis by reducing the body's immune response to an infection.

Currently, there are no known small molecule inhibitors of PCSK9. The only known marketed inhibitors of PCSK9 are anti-PCSK9 antibodies. Inhibition of PCSK9 with a small molecule inhibitor therefore has the potential to be a treatment for a range of diseases, including hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, triglyceride-rich lipoproteins (TRL), elevated triglycerides, sepsis, xanthoma and other disorders. For these reasons, there remains a need for novel and potent small molecule PCSK9 inhibitors.

### SUMMARY OF THE DISCLOSURE

A first aspect of the disclosure relates to compounds of Formula (I):

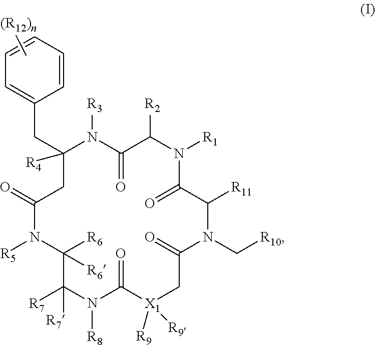

wherein:

X₁ is C or N;

R₁ is H, (C₁-C₆)alkyl, or (C₁-C₆)haloalkyl; or

R₁ and R₁₁ together with the atoms to which they are attached form a 5- to 7-membered heterocyclyl ring comprising 1-3 heteroatoms selected from N, O, and S optionally substituted with one or more substituents each independently selected from ═(O), (C₁-C₆)alkyl, and (C₁-C₆)haloalkyl;

R₂ is (C₁-C₆)alkyl, (C₁-C₆)haloalkyl, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one or more substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S;

R₃, R₄, and R₅ are each independently H, (C₁-C₆)alkyl, or (C₁-C₆)haloalkyl;

R₆, R_{6′}, R₇, and R_{7′} are each independently H, (C₁-C₆)alkyl, (C₁-C₆)haloalkyl, (C₁-C₆)hydroxyalkyl, —C(O)OH, or —C(O)O(C₁-C₆)alkyl, (C₁-C₆)hydroxyalkyl, wherein the alkyl is optionally substituted with one or more substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S; or

R₆ and R₇ together with the carbon atoms to which they are attached form a (C₃-C₇)cycloalkyl or a 4- to 7-membered heterocyclyl ring comprising 1-3 heteroatoms selected from N, O, and S; or

R₇ and R_{7′} together with the carbon atom to which they are attached form a (C₃-C₇)cycloalkyl or a 4- to 7-membered heterocyclyl ring comprising 1-3 heteroatoms selected from N, O, and S; or

R₇ and R₉ together with the atoms to which they are attached form a 5- to 7-membered heterocyclyl ring comprising 1-3 heteroatoms selected from N, O, and S optionally substituted with one or more substituents each independently selected from (C₁-C₆)alkyl, (C₁-C₆)haloalkyl, and ═(O);

R₈ is H or (C₁-C₆)alkyl;

R₉ and R_{9′} are each independently H, (C₁-C₆)alkyl, (C₂-C₆)alkenyl, (C₁-C₆)haloalkyl, (C₂-C₆)haloalkenyl, (C₁-C₆)alkoxy, (C₁-C₆)haloalkoxy, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one or more R₂₇; or R_{9′} is absent when X₁ is N; or

R₉ and R_{9′} together with the carbon atom to which they are attached form a (C₃-C₇)cycloalkyl or a 4- to 7-membered heterocyclyl ring comprising 1-3 heteroatoms selected from N, O, and S; or

R₇ and R₉ together with the atoms to which they are attached form a 5- to 7-membered heterocyclyl ring comprising 1-3 heteroatoms selected from N, O, and S optionally substituted with one or more substituents each independently selected from (C₁-C₆)alkyl, (C₁-C₆)haloalkyl, and ═(O);

R₁₀ is (C₆-C₁₀)aryl, 5- or 6-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl, heterocyclyl are substituted with —OR₁₃ or —NR₂₃R₁₃, and optionally substituted with one or more R₁₄;

R₁₁ is (C₁-C₆)alkyl, (C₁-C₆)haloalkyl, or (C₁-C₆)hydroxyalkyl, wherein the alkyl is optionally substituted with one or more R₁₅; or

R₁ and R₁₁ together with the atoms to which they are attached form a 5- to 7-membered heterocyclyl ring comprising 1-3 heteroatoms selected from N, O, and S optionally substituted with one or more substituents each independently selected from ═(O), (C₁-C₆)alkyl, and (C₁-C₆)haloalkyl;

R₁₂ is halogen, (C₁-C₆)alkyl, (C₁-C₆)alkoxy, (C₁-C₆)haloalkyl, (C₁-C₆)haloalkoxy, —OH, or CN;

R₁₃ is (C₆-C₁₀)aryl or 5- or 6-membered heteroaryl comprising 1-3 heteroatoms selected from N, O, and S, wherein the aryl and heteroaryl are substituted with R₁₆ and optionally substituted with one or more R_{16′};

each R₁₄ is independently at each occurrence halogen, (C₁-C₆)alkyl, (C₁-C₆)alkoxy, (C₁-C₆)haloalkyl, (C₁-C₆)haloalkoxy, oxo, —OH, or CN; or

when R₁₀ is cycloalkyl or heterocyclyl, two R₁₄ together, when attached to the same carbon atom, together form ═(O);

each R₁₅ is independently at each occurrence (C₁-C₆)alkoxy, (C₁-C₆)haloalkoxy, —C(O)R₁₉, —S(O)_{q}(C₁-C₆)alkyl, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, —NR₁₇C(O)OR₁₈, (C₃-C₇)cycloalkyl, or 4- to 7-membered heterocyclyl comprising 1-3 heteroatoms selected from N, O, and S, wherein the cycloalkyl and heterocyclyl are optionally substituted with one or more R₂₁;

R₁₆ is —C(O)NR₃₁R₃₂, (C₆-C₁₀)aryl, or 5- to 7-membered heteroaryl comprising 1-3 heteroatoms selected from N, O, and S, wherein the aryl and heteroaryl are optionally substituted with one or more R₂₆;

each R_{16′} is independently at each occurrence halogen, (C₁-C₆)alkyl, (C₁-C₆)alkoxy, (C₁-C₆)haloalkyl, (C₁-C₆)haloalkoxy, —OH, or CN; or

R₁₆ and R_{16′} together with the atoms to which they are attached form a 5- to 7-membered heterocyclyl ring optionally substituted with one or more substituents independently selected from ═(O) and R₃₄;

R₁₇ and R₁₈ are each independently H or (C₁-C₆)alkyl optionally substituted with one or more substituents each independently selected from (C₁-C₆)alkoxy and —C(O)O(C₁-C₆)alkyl;

R₁₉ is (C₃-C₇)cycloalkyl or 4- to 7-membered heterocyclyl comprising 1-3 heteroatoms selected from N, O, and S, wherein the cycloalkyl and heterocyclyl are optionally substituted with one or more R₂₂;

R₂₀ is —(CH₂CH₂O)ₘCH₂CH₂ONH₂, —(CH₂CH₂O)ₘCH₂CH₂ONH(C₁-C₆)alkyl, or (C₁-C₆)alkyl optionally substituted with one or more —NR₂₃C(O)R₂₄;

each R₂₁ is independently at each occurrence (C₁-C₆)alkyl, (C₁-C₆)alkoxy, (C₁-C₆)haloalkyl, (C₁-C₆)haloalkoxy, halogen, ═(O), or —OH;

each R₂₂ is independently at each occurrence (C₁-C₆)alkyl, (C₁-C₆)haloalkyl, halogen, or —OH; or

two R₂₂, when on the same atom, together with the atom to which they are attached form a (C₃-C₇)spirocycloalkyl or a 4- to 7-membered spiroheterocyclyl ring comprising 1-3 heteroatoms selected from N, O, and S;

R₂₃ is H or (C₁-C₆)alkyl;

R₂₄ is H or (C₁-C₆)alkyl optionally substituted with one or more R₂₅;

each R₂₅ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with one or more substituents each independently selected from (C₁-C₆)alkyl, (C₁-C₆)haloalkyl, and ═(O);

each R₂₆ is independently at each occurrence (C₁-C₆)alkyl optionally substituted with one or more R₂₉, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one or more substituents each independently selected from (C₁-C₆)alkyl, (C₁-C₆)haloalkyl, —NH₂, —N(H)(C₁-C₆)alkyl, —N((C₁-C₆)alkyl)₂, —N(H)(C₁-C₆)haloalkyl, —N((C₁-C₆)haloalkyl)₂, halogen, and —OH; or

two R₂₆, when on adjacent atoms, together with the atoms to which they are attached form a (C₃-C₇)carbocyclyl or a 4- to 7-membered heterocyclyl ring comprising 1-3 heteroatoms selected from N, O, and S, optionally substituted with one or more R₃₃;

each R₂₇ is independently at each occurrence CN, (C₆-C₁₀)aryl, 5- to 7-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl and heterocyclyl are optionally substituted with one or more R₂₈;

each R₂₈ is independently at each occurrence (C₁-C₆)alkyl, (C₁-C₆)haloalkyl, (C₁-C₆)alkoxy, (C₁-C₆)haloalkoxy, (C₁-C₆)hydroxyalkyl, halogen, oxo, or CN; or

when R₂₇ is cycloalkyl or heterocyclyl, two R₂₈ together with the atoms to which they are attached form a (C₄-C₇)cycloalkyl or a 4- to 7-membered heterocyclyl ring comprising 1-3 heteroatoms selected from N, O, and S; or

when R₂₇ is cycloalkyl or heterocyclyl, two R₂₈ together, when attached to the same carbon atom, together form ═(O);

each R₂₉ is independently at each occurrence —NR₃₁R₃₂ or 4- to 7-membered heterocyclyl comprising 1-3 heteroatoms selected from N, O, and S optionally substituted with one or more R₃₀;

each R₃₀ is independently at each occurrence —OH, halogen, (C₁-C₆)alkyl, or (C₁-C₆)haloalkyl; or

two R₃₀, when on the same atom, together with the atom to which they are attached form a (C₃-C₇)spirocycloalkyl or a 4- to 7-membered spiroheterocyclyl ring comprising 1-3 heteroatoms selected from N, O, and S;

R₃₁ and R₃₂ are each independently H, (C₁-C₆)alkyl, (C₁-C₆)hydroxyalkyl, (C3-C₇) cycloalkyl, or 4- to 7-membered heterocyclyl comprising 1-3 heteroatoms selected from N, O, and S, wherein the alkyl is optionally substituted with one or more D, and the cycloalkyl and heterocyclyl are optionally substituted with one or more substituents each independently selected from (C₁-C₆)alkyl, (C₁-C₆)haloalkyl, halogen, and —OH;

each R₃₃ is independently at each occurrence (C₁-C₆)alkyl, (C₁-C₆)haloalkyl, or —C(O)R, wherein R is (C₁-C₆)haloalkyl, (C₃-C₇)cycloalkyl, 4- to 7-membered heterocyclyl comprising 1-3 heteroatoms selected from N, O, and S, or (C₁-C₆)alkyl optionally substituted with one or more (C₁-C₆)alkoxy; or

two R₃₃, when on the same atom, together with the atom to which they are attached form a (C₃-C₇)spirocycloalkyl or a 4- to 7-membered spiroheterocyclyl ring comprising 1-3 heteroatoms selected from N, O, and S;

each R₃₄ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with (C₁-C₆)alkyl optionally substituted with one or more substituents each independently selected from (C₃-C₇)cycloalkyl and 4- to 10-membered mono or bicyclic heterocyclyl comprising 1-4 heteroatoms selected from N, O, and S;

m is 1-13;

n is 1, 2, 3, or 4; and

q is 0, 1, or 2;

or a pharmaceutically acceptable salt, hydrate, solvate, stereoisomer, or tautomer thereof.

In one embodiment, the present disclosure relates to compounds of Formula (I): wherein:

X₁ is C or N;

R₁ is H, (C₁-C₆)alkyl, or (C₁-C₆)haloalkyl; or

R₁ and R₁₁ together with the atoms to which they are attached form a 5- to 7-membered heterocyclyl ring comprising 1-3 heteroatoms selected from N, O, and S optionally substituted with one or more substituents each independently selected from ═(O), (C₁-C₆)alkyl, and (C₁-C₆)haloalkyl;

R₂ is (C₁-C₆)alkyl, (C₁-C₆)haloalkyl, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one or more substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S;

R₃, R₄, and R₅ are each independently H, (C₁-C₆)alkyl, or (C₁-C₆)haloalkyl;

R₆, R_{6′}, R₇, and R_{7′} are each independently H, (C₁-C₆)alkyl, (C₁-C₆)haloalkyl, or (C₁-C₆)hydroxyalkyl, wherein the alkyl is optionally substituted with one or more substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S; or

R₆ and R₇ together with the carbon atoms to which they are attached form a (C₃-C₇)cycloalkyl or a 4- to 7-membered heterocyclyl ring comprising 1-3 heteroatoms selected from N, O, and S; or

R₇ and R_{7′} together with the carbon atom to which they are attached form a (C₃-C₇)cycloalkyl or a 4- to 7-membered heterocyclyl ring comprising 1-3 heteroatoms selected from N, O, and S; or

R₇ and R₉ together with the atoms to which they are attached form a 5- to 7-membered heterocyclyl ring comprising 1-3 heteroatoms selected from N, O, and S optionally substituted with one or more substituents independently selected from (C₁-C₆)alkyl, (C₁-C₆)haloalkyl, and ═(O);

R₈ is H or (C₁-C₆)alkyl;

R₉ and R_{9′} are each independently H, (C₁-C₆)alkyl, (C₂-C₆)alkenyl, (C₁-C₆)haloalkyl, (C₂-C₆)haloalkenyl, (C₁-C₆)alkoxy, (C₁-C₆)haloalkoxy, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one or more R₂₇; or R_{9′} is absent when X₁ is N; or

R₉ and R_{9′} together with the carbon atom to which they are attached form a (C₃-C₇)cycloalkyl or a 4- to 7-membered heterocyclyl ring comprising 1-3 heteroatoms selected from N, O, and S; or

R₇ and R₉ together with the atoms to which they are attached form a 5- to 7-membered heterocyclyl ring comprising 1-3 heteroatoms selected from N, O, and S optionally substituted with one or more substituents independently selected from (C₁-C₆)alkyl, (C₁-C₆)haloalkyl, and ═(O);

R₁₀ is (C₆-C₁₀)aryl, 5- or 6-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the cycloalkyl, heterocyclyl, aryl and heteroaryl are substituted with —OR₁₃ or —NR₂₃R₁₃, and optionally substituted with one or more R₁₄;

R₁₁ is (C₁-C₆)alkyl, (C₁-C₆)haloalkyl, or (C₁-C₆)hydroxyalkyl, wherein the alkyl is optionally substituted with one or more R₁₅; or

R₁ and R₁₁ together with the atoms to which they are attached form a 5- to 7-membered heterocyclyl ring comprising 1-3 heteroatoms selected from N, O, and S optionally substituted with one or more substituents each independently selected from ═(O), (C₁-C₆)alkyl, and (C₁-C₆)haloalkyl;

R₁₂ is halogen, (C₁-C₆)alkyl, (C₁-C₆)alkoxy, (C₁-C₆)haloalkyl, (C₁-C₆)haloalkoxy, —OH, or CN;

R₁₃ is (C₆-C₁₀)aryl, or 5- or 6-membered heteroaryl comprising 1-3 heteroatoms selected from N, O, and S, wherein the aryl and heteroaryl are substituted with R₁₆ and optionally substituted with one or more R_{16′};

each R₁₄ is independently at each occurrence halogen, (C₁-C₆)alkyl, (C₁-C₆)alkoxy, (C₁-C₆)haloalkyl, (C₁-C₆)haloalkoxy, oxo, —OH, or CN; or

when R₁₀ is cycloalkyl or heterocyclyl, two R₁₄ together, when attached to the same carbon atom, together form ═(O);

each R₁₅ is independently at each occurrence (C₁-C₆)alkoxy, (C₁-C₆)haloalkoxy, —C(O)R₁₉, —S(O)_{q}(C₁-C₆)alkyl, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, —NR₁₇C(O)OR₁₈, (C₃-C₇)cycloalkyl, or 4- to 7-membered heterocyclyl comprising 1-3 heteroatoms selected from N, O, and S, wherein the cycloalkyl and heterocyclyl are optionally substituted with one or more R₂₁;

R₁₆ is —C(O)NR₃₁R₃₂, (C₆-C₁₀)aryl, or 5- to 7-membered heteroaryl comprising 1-3 heteroatoms selected from N, O, and S, wherein the aryl and heteroaryl are optionally substituted with one or more R₂₆;

each R_{16′} is independently at each occurrence halogen, (C₁-C₆)alkyl, (C₁-C₆)alkoxy, (C₁-C₆)haloalkyl, (C₁-C₆)haloalkoxy, —OH, or CN; or

R₁₆ and R_{16′} together with the atoms to which they are attached form a 5- to 7-membered heterocyclyl ring optionally substituted with one or more substituents independently selected from ═(O) and R₃₄;

R₁₇ and R₁₈ are each independently H or (C₁-C₆)alkyl optionally substituted with one or more substituents each independently selected from (C₁-C₆)alkoxy and —C(O)O(C₁-C₆)alkyl;

R₁₉ is (C₃-C₇)cycloalkyl or 4- to 7-membered heterocyclyl comprising 1-3 heteroatoms selected from N, O, and S, optionally substituted with one or more R₂₂;

R₂₀ is —(CH₂CH₂O)ₘCH₂CH₂ONH₂, —(CH₂CH₂O)ₘCH₂CH₂ONH(C₁-C₆)alkyl, or (C₁-C₆)alkyl optionally substituted with one or more —NR₂₃C(O)R₂₄;

each R₂₁ is independently at each occurrence (C₁-C₆)alkyl, (C₁-C₆)alkoxy, (C₁-C₆)haloalkyl, (C₁-C₆)haloalkoxy, halogen, ═(O), or —OH;

each R₂₂ is independently at each occurrence (C₁-C₆)alkyl, (C₁-C₆)haloalkyl, halogen, or —OH; or

two R₂₂, when on the same atom, together with the atom to which they are attached form a (C₃-C₇)spirocycloalkyl or a 4- to 7-membered spiroheterocyclyl ring comprising 1-3 heteroatoms selected from N, O, and S;

R₂₃ is H or (C₁-C₆)alkyl;

R₂₄ is H or (C₁-C₆)alkyl optionally substituted with one or more R₂₅;

each R₂₅ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with one or more substituents independently selected from (C₁-C₆)alkyl, (C₁-C₆)haloalkyl, and ═(O);

each R₂₆ is independently at each occurrence (C₁-C₆)alkyl optionally substituted with one or more R₂₉, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one or more substituents each independently selected from (C₁-C₆)alkyl, (C₁-C₆)haloalkyl, —NH₂, —N(H)(C₁-C₆)alkyl, —N((C₁-C₆)alkyl)₂, —N(H)(C₁-C₆)haloalkyl, —N((C₁-C₆)haloalkyl)₂, halogen, and —OH; or

two R₂₆, when on adjacent atoms, together with the atoms to which they are attached form a (C₃-C₇)carbocyclyl or a 4- to 7-membered heterocyclyl ring comprising 1-3 heteroatoms selected from N, O, and S, optionally substituted with one or more R₃₃;

each R₂₇ is independently at each occurrence CN, (C₆-C₁₀)aryl, 5- to 7-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl and heterocyclyl are optionally substituted with one or more R₂₈;

each R₂₈ is independently at each occurrence (C₁-C₆)alkyl, (C₁-C₆)haloalkyl, (C₁-C₆)alkoxy, (C₁-C₆)haloalkoxy, (C₁-C₆)hydroxyalkyl, halogen, oxo, or CN; or

when R₂₇ is cycloalkyl or heterocyclyl, two R₂₈ together with the atoms to which they are attached form a (C₄-C₇)cycloalkyl or a 4- to 7-membered heterocyclyl ring comprising 1-3 heteroatoms selected from N, O, and S; or

when R₂₇ is cycloalkyl or heterocyclyl, two R₂₈ together, when attached to the same carbon atom, together form ═(O);

each R₂₉ is independently at each occurrence —NR₃₁R₃₂ or 4- to 7-membered heterocyclyl comprising 1-3 heteroatoms selected from N, O, and S optionally substituted with one or more R₃₀;

each R₃₀ is independently at each occurrence —OH, halogen, (C₁-C₆)alkyl, or (C₁-C₆)haloalkyl; or

two R₃₀, when on the same atom, together with the atom to which they are attached form a (C₃-C₇)spirocycloalkyl or 4- to 7-membered spiroheterocyclyl ring comprising 1-3 heteroatoms selected from N, O, and S;

R₃₁ and R₃₂ are each independently H, (C₁-C₆)alkyl, (C₁-C₆)hydroxyalkyl, (C₃-C₇) cycloalkyl, or 4- to 7-membered heterocyclyl comprising 1-3 heteroatoms selected from N, O, and S, wherein the alkyl is optionally substituted with one or more D, and the cycloalkyl and heterocyclyl are optionally substituted with one or more substituents each independently selected from (C₁-C₆)alkyl, (C₁-C₆)haloalkyl, halogen, and —OH;

each R₃₃ is independently at each occurrence (C₁-C₆)alkyl, (C₁-C₆)haloalkyl, or —C(O)R, wherein R is (C₁-C₆)haloalkyl, (C₃-C₇)cycloalkyl, 4- to 7-membered heterocyclyl comprising 1-3 heteroatoms selected from N, O, and S, or (C₁-C₆)alkyl optionally substituted with one or more (C₁-C₆)alkoxy;

two R₃₃, when on the same atom, together with the atom to which they are attached form a (C₃-C₇)spirocycloalkyl or a 4- to 7-membered spiroheterocyclyl ring comprising 1-3 heteroatoms selected from N, O, and S;

each R₃₄ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with (C₁-C₆)alkyl optionally substituted with one or more substituents each independently selected from (C₃-C₇)cycloalkyl and 4- to 10-membered mono or bicyclic heterocyclyl comprising 1-4 heteroatoms selected from N, O, and S;

m is 1-13;

n is 1, 2, 3, or 4; and

q is 0, 1, or 2;

or pharmaceutically acceptable salts, hydrates, solvates, stereoisomers, N-oxides, or tautomers thereof.

Another aspect of the present disclosure relates to a compound of Formula (I), or a pharmaceutically acceptable salt thereof, for use in the treatment, prevention, amelioration or delay of progression of a PCSK9-mediated disease or disorder or for use in the treatment, prevention, amelioration or delay of progression of a disease or disorder requiring inhibition of PCSK9 or of PCSK9 activity

In another aspect, the present disclosure relates to the use of a compound of Formula (I), or a pharmaceutically acceptable salt thereof, for the treatment, prevention, amelioration or delay of progression of a PCSK9-mediated disease or disorder or for the treatment, prevention, amelioration or delay of progression of a disease or disorder requiring inhibition of PCSK9 or of PCSK9 activity.

Another aspect of the present disclosure relates to the use of a compound of Formula (I), or a pharmaceutically acceptable salt thereof, in the manufacture of a medicament for the treatment, prevention, amelioration or delay of progression of a PCSK9-mediated disease or disorder or for the treatment, prevention, amelioration or delay of progression of a disease or disorder requiring inhibition of PCSK9 or of PCSK9 activity.

In another aspect, the present disclosure relates a method for treating, preventing, ameliorating or delaying the progression of a PCSK9-mediated disease or disorder comprising the step of administering to a patient in need thereof a therapeutically effective amount of a compound of Formula (I), or a pharmaceutically acceptable salt thereof, according to the disclosure, or a pharmaceutically acceptable salt thereof.

Another aspect of the present disclosure relates to a method for treating, preventing, ameliorating or delaying the progression of a PCSK9-mediated disease or a disorder or of disease or disorder requiring inhibition of PCSK9 or of PCSK9 activity comprising the step of administering to a patient in need thereof a therapeutically effective amount of a compound of Formula (I), or a pharmaceutically acceptable salt thereof, according to the disclosure, or a pharmaceutically acceptable salt thereof.

In another aspect, the present disclosure relates a method of treating, preventing, inhibiting, or eliminating hypercholesterolemia, hyperlipidemia, hypertriglyceridemia, sitosterolemia, atherosclerosis, arteriosclerosis, coronary heart disease, peripheral vascular disease, vascular inflammation, xanthoma, peripheral arterial disease, sepsis, elevated Lp(a), elevated LDL, elevated TRL, or elevated triglycerides comprising the step of administering to a patient in need thereof a therapeutically effective amount of a compound of Formula (I), or a pharmaceutically acceptable salt thereof.

Another aspect of the present disclosure relates to a method of (i) reducing Lp(a), (ii) reducing Lp(a) plasma levels, (iii) reducing Lp(a) serum levels, (iv) reducing serum TRL or LDL levels, (v) reducing serum triglyceride levels, (vi) reducing LDL-C, (vii) reducing total plasma apoB concentrations, (viii) reducing LDL apoB, (ix) reducing TRL apoB, or (x) reducing non HDL-C comprising the step of administering to a patient in need thereof a therapeutically effective amount of a compound of Formula (I), or a pharmaceutically acceptable salt thereof.

In another aspect, the present disclosure also relates to a method of (i) reducing LDL-C, (ii) reducing total apolipoprotein B (apoB) concentrations, (iii) reducing LDL apoB, (iv) reducing TRL apoB, or (v) reducing non HDL-C and combinations thereof, in a patient in need thereof, wherein the method comprising administering a therapeutically effective amount of a compound of Formula (I), or a pharmaceutically acceptable salt thereof, to the patient.

Another aspect of the present disclosure relates to a method of reducing the total plasma concentration of a marker selected from (i) LDL-C, (ii) apoB, (iii) LDL apoB, (iv) TRL apoB and (v) non HDL-C and combinations thereof, in a patient in need thereof, wherein the method comprising administering a therapeutically effective amount of a compound of Formula (I), or a pharmaceutically acceptable salt thereof, to the patient.

In another aspect, the present disclosure relates to a pharmaceutical composition comprising (e.g., a therapeutically effective amount of) a compound of Formula (I), or a pharmaceutically acceptable salt thereof, and one or more pharmaceutically acceptable carriers or excipients.

Another aspect of the present disclosure relates to a pharmaceutical composition comprising (e.g., a therapeutically effective amount of) a compound of Formula (I), or a pharmaceutically acceptable salt thereof, and one or more pharmaceutically acceptable carriers or excipients for use in the treatment of a PCSK9-mediated disease or disorder.

In another aspect, the present disclosure relates to a method of modulating PCSK9 comprising administering to a patient in need thereof a compound of Formula (I), or a pharmaceutically acceptable salt thereof.

Another aspect of the present disclosure relates to a method of inhibiting PCSK9 comprising administering to a patient in need thereof a compound of Formula (I), or a pharmaceutically acceptable salt thereof. In another aspect, the present disclosure relates to a method of inhibiting PCSK9 activity comprising administering to a patient in need thereof a compound of Formula (I), or a pharmaceutically acceptable salt thereof.

In another aspect, the present disclosure relates to a method for treating a PCSK9-mediated disease or disorder comprising the step of administering to a patient in need thereof a therapeutically effective amount of a compound of Formula (I), or a pharmaceutically acceptable salt thereof.

Another aspect of the present disclosure relates to a method of reducing LDL-C in a patient in need thereof, the method comprising administering a therapeutically effective amount of a compound of Formula (I), or a pharmaceutically acceptable salt thereof to the patient, thereby reducing LDL-C in the patient.

In another aspect, the present disclosure relates to a compound of Formula (I), or a pharmaceutically acceptable salt thereof, for use in the treatment of a PCSK9-mediated disease or disorder.

Another aspect of the present disclosure relates to a compound of Formula (I), or a pharmaceutically acceptable salt thereof, for use in the treatment of a PCSK9-mediated disease or disorder which is selected from hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma.

In another aspect, the present disclosure relates to the use of a compound of Formula (I), or a pharmaceutically acceptable salt thereof, or a pharmaceutically acceptable salt thereof, in the manufacture of a medicament for treating a PCSK9-mediated disease or disorder.

Another aspect of the present disclosure relates to a compound of Formula (I), or a pharmaceutically acceptable salt thereof, for use in the manufacture of a medicament for treating a disease associated with inhibiting PCSK9 activity.

In another aspect, the present disclosure relates to the use of a compound of Formula (I), or a pharmaceutically acceptable salt thereof, in the treatment of a disease associated with the inhibition of PCSK9 activity.

In certain aspects, the PCSK9 modulating or inhibiting compounds of the disclosure may be administered alone or in combination with other compounds, including other PCSK9 modulating or inhibiting agents, or other therapeutic agents.

Accordingly, in another aspect, the present disclosure relates to a combination, comprising (e.g. a therapeutically effective amount of) a compound of formula (I), or a pharmaceutically acceptable salt thereof, and one or more therapeutically active agents.

Another aspect of the present disclosure relates to a process for the manufacture of a compound of Formula (II), or pharmaceutically acceptable salts, hydrates, solvates, stereoisomers, N-oxides, or tautomers thereof,

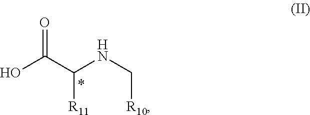

wherein R₁₀ and R₁₁ are as defined above for Formula (I), and * denotes a chiral center, comprising reacting a compound of Formula (IIa), or pharmaceutically acceptable salts, hydrates, solvates, stereoisomers, N-oxides, or tautomers thereof,

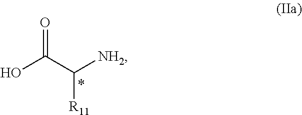

wherein R₁₁ is as defined above for Formula (I) and * denotes a chiral center; with a compound of Formula (IIb), or pharmaceutically acceptable salts, hydrates, solvates, stereoisomers, N-oxides, or tautomers thereof,

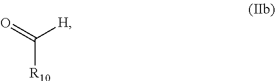

wherein R₁₀ is as defined above for Formula (I), or pharmaceutically acceptable salts, hydrates, solvates, stereoisomers, N-oxides, or tautomers thereof, via a reductive amination in the presence of a reducing agent, in aliphatic alcohol solvent, and at low temperature, to obtain the compound of Formula (II) in >70% enantiomeric excess (ee). In one embodiment, the reducing agent is sodium triacetoxyborohydride, sodium cyanoborohydride, or sodium borohydride. In another embodiment, the reducing agent is sodium borohydride. In one embodiment, the reductive amination is run at a temperature ≤10° C., ≤9° C., ≤8° C., ≤7° C., ≤6° C., ≤5° C., ≤4° C., ≤3° C., ≤2° C., ≤1° C., ≤0° C., ≤−1° C., ≤−2° C., ≤−3° C., ≤−4° C., ≤−5° C., ≤−6° C., ≤−7° C., ≤−8° C., ≤−9° C., ≤−10° C., ≤−11° C., ≤−12° C., ≤−13° C., ≤−14° C., ≤−15° C., ≤−16° C., ≤−17° C., ≤−18° C., ≤−19° C., or ≤−20° C. In one embodiment, the reductive amination is run at a temperature <10° C., <9° C., <8° C., <7° C., <6° C., <5° C., <4° C., <3° C., <2° C., <1° C., <0° C., <−1° C., <−2° C., <−3° C., <−4° C., <−5° C., <−6° C., <−7° C., <−8° C., <−9° C., <−10° C., <−11° C., <−12° C., <−13° C., <−14° C., <−15° C., <−16° C., <−17° C., <−18° C., <−19° C., or <−20° C. In another embodiment, the reductive amination is run at a temperature between about 5° C. to about 10° C., between about 1° C. to about 5° C., between about 0° C. to about 5° C., between about −5° C. to about 0° C., between about −10° C. to about −5° C., between about −10° C. to about −15° C., between about −15° C. to about −20° C. In another embodiment, the reductive amination is run at temperature of about 10° C., about 9° C., about 8° C., about 7° C., about 6° C., about 5° C., about 4° C., about 3° C., about 2° C., about 1° C., about 0° C., about −1° C., about −2° C., about −3° C., about −4° C., about −5° C., about −6° C., about −7° C., about −8° C., about −9° C., about −10° C., about −11° C., about −12° C., about −13° C., about −14° C., about −15° C., about −16° C., about −17° C., about −18° C., about −19° C., or about −20° C.

In one embodiment, the aliphatic alcohol solvent is methanol, ethanol, or i-propanol. In another embodiment, the aliphatic alcohol solvent is methanol. In one embodiment, the compound of Formula (II) is obtained in ≥70% ee, ≥75% ee, ≥80% ee, ≥90% ee, ≥95% ee, ≥96% ee, ≥97% ee, ≥98% ee, or ≥99% ee. In another embodiment, the compound of Formula (II) is obtained in >70% ee, >75% ee, >80% ee, >90% ee, >95% ee, >96% ee, >97% ee, >98% ee, or >99% ee. In another embodiment, the compound of Formula (II) is obtained in about 70% ee, about 75% ee, about 80% ee, about 90% ee, about 95% ee, about 96% ee, about 97% ee, about 98% ee, or about 99% ee. In another embodiment, the reducing agent is sodium borohydride, and the temperature is about −5° C. In another embodiment, the reducing agent is sodium borohydride, the temperature is about −5° C., and the aliphatic alcohol solvent is methanol. In another embodiment, the reducing agent is sodium borohydride, the temperature is about −5° C., the aliphatic alcohol solvent is methanol, and the compound of Formula (II) is obtained in >99% ee. In one embodiment of the compound of Formula (II), R₁₀ is (C₆-C₁₀)aryl or 5- or 6-membered heteroaryl comprising 1-3 heteroatoms selected from N, O, and S, the aryl and heteroaryl are substituted with —OR₁₃, and optionally substituted with one or more R₁₄; and R₁₃ is (C₆-C₁₀)aryl, or 5- or 6-membered heteroaryl comprising 1-3 heteroatoms selected from N, O, and S, wherein the aryl and heteroaryl are substituted with R₁₆ and optionally substituted with one or more R_{16′}.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar to or equivalent to those described herein can be used in the practice and testing of the disclosure, suitable methods and materials are described below. All publications, patent applications, patents, and other references mentioned herein are incorporated by reference. The references cited herein are not admitted to be prior art to the claimed disclosure. In the case of conflict, the present specification, including definitions, will control. In addition, the materials, methods, and examples are illustrative only and not intended to be limiting.

Other features and advantages of the disclosure will be apparent from the following detailed description and claims.

### DETAILED DESCRIPTION OF THE DISCLOSURE

The present disclosure relates to compounds and compositions that are capable of modulating the activity of PCSK9. The disclosure features methods of treating, preventing or ameliorating a disease or disorder in which PCSK9 plays a role by administering to a patient in need thereof a therapeutically effective amount of a compound of Formula (I), or a pharmaceutically acceptable salt, hydrate, solvate, stereoisomer, or tautomer thereof. The methods of the present disclosure can be used in the treatment of a variety of PCSK9 dependent diseases and disorders by modulating or inhibiting PCSK9. Inhibition or modulation of PCSK9 provides a novel approach to the treatment, prevention, or amelioration of diseases including, but not limited to, hypercholesterolemia, hyperlipidemia, hypertriglyceridemia, sitosterolemia, atherosclerosis, arteriosclerosis, coronary heart disease, peripheral vascular disease (including aortic diseases and cerebrovascular disease), peripheral arterial disease, vascular inflammation, elevated Lp(a), elevated LDL, elevated TRL, elevated triglycerides, sepsis, and xanthoma.

The compounds of the disclosure, by inhibiting PCSK9, have utility in the treatment of hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL (e.g., elevated VLDL and/or chylomicrons), elevated triglycerides, sepsis, and xanthoma.

For example, the compounds of Formula (I) of the disclosure bind to PCSK9 and thereby inhibit PCSK9 and/or PCSK9 activity, since PCSK9 cannot any longer bind to the low density lipoprotein receptors (LDLR) or any other target receptors. For example, if PCSK9 is blocked, more LDLRs are recycled and are present on the surface of cells to remove LDL-particles from the extracellular fluid. Therefore, blocking PCSK9 can lower blood LDL-particle concentrations.

Accordingly, compounds of the present disclosure may therefore be potentially useful in the treatment, prevention, amelioration or delay of progression of a PCSK9-mediated disease or disorder, or a disease or disorder in which PCSK9 plays a role, as well as conditions, diseases and disorders benefitting from modulating PCSK9 or PCSK9 activity.

In addition, compounds of the present disclosure may therefore be potentially useful in the treatment, prevention, amelioration or delay of progression of a disease or disorder requiring inhibition of PCSK9 or of PCSK9 activity.

Such diseases and disorders include diseases or disorders selected from hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL (e.g., elevated VLDL and/or chylomicrons), elevated triglycerides, sepsis, and xanthoma.

Various embodiments of the disclosure are described herein. It will be recognized that features specified in each embodiment may be combined with other specified features of other embodiments to provide further embodiments.

In a first aspect of the disclosure, the compounds of Formula (I) are described:

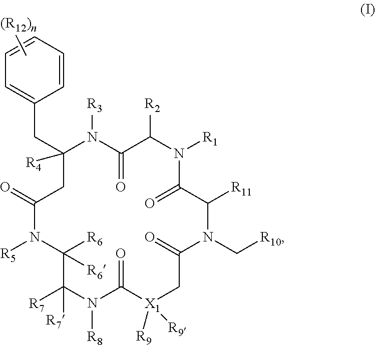

and pharmaceutically acceptable salts, hydrates, solvates, stereoisomers, and tautomers thereof, wherein X₁, R₁, R₂, R₃, R₄, R₅, R₆, R_{6′}, R₇, R_{7′}, R₈, R₉, R_{9′}, R₁₀, R₁₁, R₁₂, and n are as described herein above.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

### Chemical Nomenclature, Terms, and Conventions

In the groups, radicals, or moieties defined below, the number of carbon atoms is often specified preceding the group, for example, (C₁-C₁₀)alkyl means an alkyl group or radical having 1 to 10 carbon atoms. In general, for groups comprising two or more subgroups, the last named group is the radical attachment point, for example, “alkylaryl” means a monovalent radical of the formula alkyl-aryl-, while “arylalkyl” means a monovalent radical of the formula aryl-alkyl-.

Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups. The articles “a” and “an” are used in this disclosure to refer to one or more than one (e.g., to at least one) of the grammatical object of the article. By way of example, “an element” means one element or more than one element.

The term “and/or” is used in this disclosure to mean either “and” or “or” unless indicated otherwise.

The term “optionally substituted” means that a given chemical moiety (e.g., an alkyl group) can (but is not required to) be bonded other substituents (e.g., heteroatoms). For instance, an alkyl group that is optionally substituted can be a fully saturated alkyl chain (e.g., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, ═(O), —OH, —CN, —COOH, —CH₂CN, —O—(C₁-C₆)alkyl, (C₁-C₆)alkyl, (C₁-C₆)alkoxy, (C₁-C₆)haloalkyl, (C₁-C₆)haloalkoxy, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O)(C₁-C₆)alkyl, —C(O)NH(C₁-C₆)alkyl, —S(O)₂(C₁-C₆)alkyl, —S(O)NH(C₁-C₆)alkyl, and S(O)N((C₁-C₆)alkyl)₂. The substituents can themselves be optionally substituted. “Optionally substituted” as used herein also refers to substituted or unsubstituted whose meaning is described below.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

The term “unsubstituted” means that the specified group bears no substituents.

Unless otherwise specifically defined, “aryl” means a cyclic, aromatic hydrocarbon group having 1 to 3 aromatic rings, including monocyclic or bicyclic groups such as phenyl, biphenyl, or naphthyl. When containing two aromatic rings (bicyclic, etc.), the aromatic rings of the aryl group are optionally joined at a single point (e.g., biphenyl), or fused (e.g., naphthyl). The aryl group is optionally substituted by one or more substituents, e.g., 1 to 5 substituents, at any point of attachment. Exemplary substituents include, but are not limited to, —H, -halogen, —CN, —O—(C₁-C₆)alkyl, (C₁-C₆)alkyl, —O—(C₂-C₆)alkenyl, —O—(C₂-C₆)alkynyl, (C₂-C₆)alkenyl, (C₂-C₆)alkynyl, —OH, —OP(O)(OH)₂, —OC(O)(C₁-C₆)alkyl, —C(O)(C₁-C₆)alkyl, —OC(O)O(C₁-C₆)alkyl, NH₂, NH((C₁-C₆)alkyl), N((C₁-C₆)alkyl)₂, —S(O)₂—(C₁-C₆)alkyl, —S(O)NH(C₁-C₆)alkyl, and S(O)N((C₁-C₆)alkyl)₂. The substituents are themselves optionally substituted. Furthermore, when containing two fused rings, the aryl groups optionally have an unsaturated or partially saturated ring fused with a fully saturated ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic aromatic radical of 5 to 24 ring atoms or a polycyclic aromatic radical, containing one or more ring heteroatoms selected from N, O, or S, the remaining ring atoms being C. Heteroaryl as herein defined also means a bicyclic heteroaromatic group wherein the heteroatom is selected from N, O, or S. The aromatic radical is optionally substituted independently with one or more substituents described herein. Examples include, but are not limited to, furyl, thienyl, pyrrolyl, pyridyl, pyridyl N-oxide, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, dihydrobenzoxanyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydropyrrolo[1,2-a]pyrimidinyl, 3,4-dihydro-2H-1Δ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[I,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4 d]thiazolyl, imidazo[2,1-b][1,3,4]thiadiazolyl, thieno[2,3-b]pyrrolyl, 3H-indolyl, and derivatives thereof. Furthermore, when containing two fused rings the aryl groups herein defined may have an unsaturated or partially saturated ring fused with a fully saturated ring. Exemplary ring systems of these heteroaryl groups include indolinyl, indolinonyl, dihydrobenzothiophenyl, dihydrobenzofuran, chromanyl, thiochromanyl, tetrahydroquinolinyl, dihydrobenzothiazine,3,4-dihydro-1H-isoquinolinyl, 2,3-dihydrobenzofuran, indolinyl, indolyl, and dihydrobenzoxanyl.

“Halogen” or “halo” mean fluorine, chlorine, bromine, or iodine.

“Alkyl” means a straight or branched chain saturated hydrocarbon containing 1-12 carbon atoms. Examples of a (C₁-C₆)alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

“Alkenyl” means a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted and may be straight or branched.

“Alkynyl” means a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

“Cycloalkyl” means a monocyclic or polycyclic saturated carbon ring containing 3-18 carbon atoms. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, or bicyclo[2.2.2]octenyl and derivatives thereof. A (C₃-C₈)cycloalkyl is a cycloalkyl group containing between 3 and 8 carbon atoms. A cycloalkyl group can be fused (e.g., decalin) or bridged (e.g., norbomane).

“Carbocyclyl” means a monocyclic or polycyclic saturated or partially unsaturated carbon ring containing 3-18 carbon atoms (e.g., cycloalkyl, cycloalkenyl, cycloalkynyl, etc). Examples of carbocyclyl groups include, without limitations, cyclopropyl, cyclobutyl, cyclobutenyl, cyclopentyl, cyclopentenyl, cyclohexyl, cyclohexenyl cycloheptanyl, cyclooctanyl, norboranyl, norborenyl, bicyclo[2.2.2]octanyl, or bicyclo[2.2.2]octenyl and derivatives thereof. A (C₃-C₈) carbocyclyl is a carbocyclyl group containing between 3 and 8 carbon atoms. A carbocyclyl group can be fused (e.g., decalin) or bridged (e.g., norbomane).

“Heterocycloalkyl” means a saturated monocyclic or polycyclic ring containing carbon and at least one heteroatom selected from oxygen, nitrogen, or sulfur (O, N, or S) and wherein there is not delocalized n electrons (aromaticity) shared among the ring carbon or heteroatoms. The heterocycloalkyl ring structure may be substituted by one or more substituents. The substituents can themselves be optionally substituted. Examples of heterocycloalkyl rings include, but are not limited to, oxetanyl, azetadinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, imidazolinyl, dithiolanyl, and homotropanyl.

“Heterocyclyl” means a saturated (e.g., heterocycloalkyl ring) or partially unsaturated monocyclic or polycyclic ring containing carbon and at least one heteroatom selected from oxygen, nitrogen, or sulfur (O, N, or S) and wherein there is not delocalized n electrons (aromaticity) shared among the ring carbon or heteroatoms. The heterocyclyl ring structure may be substituted by one or more substituents. The substituents can themselves be optionally substituted. Examples of heterocyclyl rings include, but are not limited to, oxetanyl, azetadinyl, tetrahydrofuranyl, tetrahydropyranyl, pyrrolidinyl, dihydropyrrolidinyl, pyridin-2(1H)-one, dihydropyridinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, imidazolinyl, dithiolanyl, and homotropanyl.

“Hydroxyalkyl” means an alkyl group substituted with one or more —OH groups. Examples of hydroxyalkyl groups include HO—CH₂—, HO—CH₂CH₂—, and CH₂—CH(OH)—.

“Haloalkyl” means an alkyl group substituted with one or more halogens. Examples of haloalkyl groups include, but are not limited to, trifluoromethyl, difluoromethyl, pentafluoroethyl, trichloromethyl, etc.

“Haloalkoxy” means an alkoxy group substituted with one or more halogens. Examples of haloalkyl groups include, but are not limited to, trifluoromethoxy, difluoromethoxy, pentafluoroethoxy, trichloromethoxy, etc.

“Cyano” means a substituent having a carbon atom joined to a nitrogen atom by a triple bond, e.g., C≡N.

The term “oxo” as used herein refers to an “—O^{⊖}” group.

The term “N-oxide” refers to an oxygen atom bound by a single bond (e.g., “oxo”) to a nitrogen atom (e.g., N—O^{⊖}).

“Amino” means a substituent containing at least one nitrogen atom (e.g., NH₂).

“Spirocycloalkyl” or “spirocyclyl” means carbogenic bicyclic ring systems with both rings connected through a single atom. The rings can be different in size and nature, or identical in size and nature. Examples include spiropentane, spirohexane, spiroheptane, spirooctane, spirononane, or spirodecane. One or both of the rings in a spirocycle can be fused to another ring carbocyclic, heterocyclic, aromatic, or heteroaromatic ring. A (C₃-C₁₂)spirocycloalkyl is a spirocycle containing between 3 and 12 carbon atoms.

“Spiroheterocycloalkyl” or “spiroheterocyclyl” means a spirocycle wherein at least one of the rings is a heterocycle one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

### Salt, Prodrug, Derivative, and Solvate Terms and Conventions

“Prodrug” or “prodrug derivative” mean a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; Bioreversible Carriers in Drug Design, E. B. Roche (ed.), Elsevier, 1987, each of which is incorporated herein by reference in their entireties.

“Pharmaceutically acceptable prodrug” as used herein means a prodrug of a compound of the disclosure which is, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible.

“Salt” means an ionic form of the parent compound or the product of the reaction between the parent compound with a suitable acid or base to make the acid salt or base salt of the parent compound. Salts of the compounds of the present disclosure can be synthesized from the parent compounds, which contain a basic or acidic moiety, by conventional chemical methods. Generally, the salts are prepared by reacting the free base or acid parent compound with stoichiometric amounts or with an excess of the desired salt-forming inorganic or organic acid or base in a suitable solvent or various combinations of solvents.

“Pharmaceutically acceptable salt” means a salt of a compound of the disclosure which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. As the compounds of the present disclosure are useful in both free base and salt form, in practice, the use of the salt form amounts to use of the base form. Lists of suitable salts are found in, e.g., S. M. Birge, et. al., J. Pharm. Sci., 1977, 66, pp. 1-19, which is hereby incorporated by reference in its entirety.

“Pharmaceutically-acceptable acid addition salt” means those salts which retain the biological effectiveness and properties of the free bases and which are not biologically or otherwise undesirable, formed with inorganic acids such as hydrochloric acid, hydrobromic acid, hydroiodic acid, sulfuric acid, sulfamic acid, nitric acid, phosphoric acid, and the like, and organic acids such as acetic acid, trichloroacetic acid, trifluoroacetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, heptanoic acid, hexanoic acid, formic acid, fumaric acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, undecanoic acid, and the like.

“Pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

“Solvate” means a complex of variable stoichiometry formed by a solute, for example, a compound of Formula (I) and solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, such solvents selected for the purpose of the disclosure do not interfere with the biological activity of the solute. Solvates encompasses both solution-phase and isolatable solvates. Representative solvates include hydrates, ethanolates, methanolates, and the like.

“Hydrate” means a solvate wherein the solvent molecule(s) is/are water. The compounds of the present disclosure as discussed below include the free base or acid thereof, their salts, solvates, and prodrugs and may include oxidized sulfur atoms or quaternized nitrogen atoms in their structure, although not explicitly stated or shown, particularly the pharmaceutically acceptable forms thereof. Such forms, particularly the pharmaceutically acceptable forms, are intended to be embraced by the appended claims.

### Isomer Terms and Conventions

“Isomers” means compounds having the same number and kind of atoms, and hence the same molecular weight, but differing with respect to the arrangement or configuration of the atoms in space. The term includes stereoisomers and geometric isomers.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

“Enantiomers” means a pair of stereoisomers that are non-superimposable mirror images of each other.

“Diastereoisomers” or “diastereomers” mean optical isomers which are not mirror images of each other.

“Racemic mixture” or “racemate” mean a mixture containing equal parts of individual enantiomers.

“Non-racemic mixture” means a mixture containing unequal parts of individual enantiomers.

“Geometrical isomer” means a stable isomer which results from restricted freedom of rotation about double bonds (e.g., cis-2-butene and trans-2-butene) or in a cyclic structure (e.g., cis-1,3-dichlorocyclobutane and trans-1,3-dichlorocyclobutane). Because carbon-carbon double (olefinic) bonds, C═N double bonds, cyclic structures, and the like may be present in the compounds of the disclosure, the disclosure contemplates each of the various stable geometric isomers and mixtures thereof resulting from the arrangement of substituents around these double bonds and in these cyclic structures. The substituents and the isomers are designated using the cis/trans convention or using the E or Z system, wherein the term “E” means higher order substituents on opposite sides of the double bond, and the term “Z” means higher order substituents on the same side of the double bond. A thorough discussion of E and Z isomerism is provided in J. March, Advanced Organic Chemistry: Reactions, Mechanisms, and Structure, 4th ed., John Wiley & Sons, 1992, which is hereby incorporated by reference in its entirety. Several of the following examples represent single E isomers, single Z isomers, and mixtures of E/Z isomers. Determination of the E and Z isomers can be done by analytical methods such as x-ray crystallography, ¹H NMR, and ¹³C NMR.

Some of the compounds of the disclosure can exist in more than one tautomeric form. As mentioned above, the compounds of the disclosure include all such tautomers.

It is well known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

Preparation of pure enantiomers or mixtures of desired enantiomeric excess (ee) or enantiomeric purity are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000. Furthermore, there are equally well-known methods for the quantitation of enantiomeric excess or purity, for example, GC, HPLC, CE, or NMR, and assignment of absolute configuration and conformation, for example, CD ORD, X-ray crystallography, or NMR.

In general, all tautomeric forms and isomeric forms and mixtures, whether individual geometric isomers or stereoisomers or racemic or non-racemic mixtures, of a chemical structure or compound is intended, unless the specific stereochemistry or isomeric form is specifically indicated in the compound name or structure.

### Pharmaceutical Administration and Treatment Terms and Conventions

A “patient” or “subject” is a mammal, e.g., a human, mouse, rat, guinea pig, dog, cat, horse, cow, pig, or nonhuman primate, such as a monkey, chimpanzee, baboon or, rhesus. In certain embodiments, the subject is a primate. In yet other embodiments, the subject is a human.

The terms “pharmaceutically effective amount” or “therapeutically effective amount” or “effective amount” means an amount of a compound according to the disclosure which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound according to the disclosure which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the disclosure, and the age, body weight, general health, sex, and diet of the patient. Such a therapeutically effective amount can be determined routinely by one of ordinary skill in the art having regard to their own knowledge, the prior art, and this disclosure.

As used herein, the term “pharmaceutical composition” refers to a compound of the disclosure, or a pharmaceutically acceptable salt, hydrate, solvate, stereoisomer, or tautomer thereof, together with at least one pharmaceutically acceptable carrier, in a form suitable for oral or parenteral administration.

“Carrier” encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

“Combination” refers to either a fixed combination in one dosage unit form, or a combined administration where a compound of the present disclosure and at least one combination partner (e.g. another drug as explained below, also referred to as “therapeutic agent” or “co-agent”) may be administered independently at the same time or separately within time intervals, especially where these time intervals allow that the combination partners show a beneficial effect from the co-action of these therapeutic agents. The beneficial effect of the combination includes, but is not limited to, a cooperative, e.g., synergistic, effect and/or a pharmacokinetic or pharmacodynamic co-action, or any combination thereof, resulting from the combination of therapeutic agents. In one embodiment, administration of these therapeutic agents in combination is carried out over a defined time period (e.g., minutes, hours, days or weeks depending upon the combination selected).

The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound of the present disclosure and a combination partner, are both administered to a patient simultaneously in the form of a single entity or dosage. The term “non-fixed combination” means that the therapeutic agents, e.g., a compound of the present disclosure and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

A subject is “in need of” a treatment if such subject would benefit biologically, medically, or in quality of life from such treatment (preferably, a human).

The term “PCSK9” or “proprotein convertase subtilisin/kexin type 9” interchangeably refer to a naturally-occurring human proprotein convertase belonging to the proteinase K subfamily of the secretory subtilase family. PCSK9 is synthesized as a soluble zymogen that undergoes autocatalytic intramolecular processing in the endoplasmic reticulum, and is thought to function as a proprotein convertase. PCSK9 plays a role in cholesterol homeostasis and may have a role in the differentiation of cortical neurons. Mutations in the PCSK9 gene are a cause of autosomal dominant familial hypercholesterolemia. (Burnett and Hooper, Clin. Biochem. Rev. (2008) 29(1):11-26)

As used herein, the term “inhibit”, “inhibition”, or “inhibiting” refers to the reduction or suppression of a given condition, symptom, or disorder, or disease, or a significant decrease in the baseline activity of a biological activity or process.

As used herein, the term “treat”, “treating”, or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing”, or “prevention” of any disease or disorder refers to the prophylactic treatment of the disease or disorder; or delaying the onset or progression of the disease or disorder.

“Pharmaceutically acceptable” means that the substance or composition must be compatible chemically and/or toxicologically, with the other ingredients comprising a formulation, and/or the mammal being treated therewith.

“Disorder” means, and is used interchangeably with, the terms disease, condition, or illness, unless otherwise indicated.

“Administer”, “administering”, or “administration” means to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

“Prodrug” means a compound which is convertible in vivo by metabolic means (e.g., by hydrolysis) to a disclosed compound.

“Compounds of the present disclosure”, “Compounds of Formula (I)”, “compounds of the disclosure”, and equivalent expressions (unless specifically identified otherwise) refer to compounds of Formula (I), (Ia), (Ia-1), (Ia-2), (Ib), (Ic), (Id), (le), (If), (Ig), (Ih), (Ii), and (Ij) as herein described including the tautomers, the prodrugs, salts particularly the pharmaceutically acceptable salts, and the solvates and hydrates thereof, where the context so permits thereof, as well as all stereoisomers (including diastereoisomers and enantiomers), rotamers, tautomers, and isotopically labelled compounds (including deuterium (“D”) substitutions), as well as inherently formed moieties (e.g., polymorphs, solvates and/or hydrates). For purposes of this disclosure, solvates and hydrates are generally considered compositions. In general and preferably, the compounds of the disclosure and the formulas designating the compounds of the disclosure are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

“Stable compound” or “stable structure” means a compound that is sufficiently robust to survive isolation to a useful degree of purity from a reaction mixture, and formulation into an efficacious therapeutic or diagnostic agent. For example, a compound, which would have a “dangling valency” or is a carbanion is not a compound contemplated by the disclosure.

In a specific embodiment, the term “about” or “approximately” means within 20%, preferably within 10%, and more preferably within 5% of a given value or range.

As used herein, a “modulator of PCSK9” refers to a compound or molecule that is able to modulate PCSK9 biological activity or function, and/or downstream pathway(s) mediated by PCSK9 activity.

As used herein, an “inhibitor of PCSK9” refers to a compound or molecule that is able to inhibit PCSK9 biological activity or function, and/or downstream pathway(s) mediated by PCSK9 signaling. An inhibitor of PCSK9 activity encompasses compounds that block, antagonize, suppress or reduce (to any degree including significantly) PCSK9 biological activity, including downstream pathways mediated by PCSK9 activity.

As used herein, “disorders or diseases responsive to the inhibition of PCSK9,” “disorders and conditions responsive to the inhibition of PCSK9,” “disorders and conditions responsive to the inhibition of PCSK9 activity,” “disorders responsive to the inhibition of PCSK9,” “disorders responsive to the inhibition of PCSK9 activity,” “disorders in which PCSK9 plays a role,” and like terms include hypercholesterolemia, hyperlipidemia, hypertriglyceridemia, sitosterolemia, atherosclerosis, arteriosclerosis, coronary heart disease, peripheral vascular disease (including aortic diseases and cerebrovascular disease), peripheral arterial disease, vascular inflammation, elevated Lp(a), elevated LDL, elevated TRL, elevated triglycerides, sepsis, and xanthoma.

As used herein, “Inhibition of PCSK9 activity,” or “inhibition of PCSK9,” refers to a decrease in the PCSK9 activity, e.g., by administration of a compound of the disclosure.

The term “hypercholesterolemia” or “dyslipidemia” includes, e.g., familial and non-familial hypercholesterolemia. Familial hypercholesterolemia (FH) is an autosomal dominant disorder characterized by elevation of serum cholesterol bound to low density lipoprotein (LDL). Familial hypercholesterolemia includes both heterozygous FH and homozygous FH.

Hypercholesterolemia (or dyslipidemia) is the presence of high levels of cholesterol in the blood. It is a form of hyperlipidemia (elevated levels of lipids in the blood) and hyperlipoproteinemia (elevated levels of lipoproteins in the blood).

Hyperlipidemia is an elevation of lipids in the bloodstream. These lipids include cholesterol, cholesterol esters, phospholipids and triglycerides. Hyperlipidemia includes for example, type I, IIa, IIb, III, IV and V.

Hypertriglyceridemia denotes high blood levels of triglycerides. Elevated levels of triglycerides are associated with atherosclerosis, even in the absence of hypercholesterolemia, and predispose to cardiovascular disease.

“Sitosterolemia” or “phytosterolemia” is a rare autosomal recessively inherited lipid metabolic disorder characterized by hyperabsorption of sitosterol from the gastrointestinal tract and decreased biliary excretion of dietary sterols (i.e., leading to hypercholesterolemia, tendon and tuberous xanthomas, premature development of atherosclerosis) and altered cholesterol synthesis.

“Atherosclerosis” includes hardening of arteries associated with deposition of fatty substances, cholesterol, cellular waste products, calcium and fibrin in the inner lining of an artery. The buildup that results is called plaque.

“Atherosclerosis” or “arteriosclerotic vascular disease (ASVD)” is a specific form of arteriosclerosis involving thickening, hardening and loss of elasticity of the walls of arteries as a result of invasion and accumulation of white blood cells, containing both living, active white blood cells (producing inflammation) and remnants of dead cells, including cholesterol and triglycerides. Atherosclerosis is therefore a syndrome affecting arterial blood vessels due to a chronic inflammatory response of white blood cells in the walls of arteries.

“Coronary heart disease,” also known as atherosclerotic artery disease, atherosclerotic cardiovascular disease, coronary heart disease or ischemic heart disease is the most common type of heart disease and cause of heart attacks. The disease is caused by plaque building up along the inner walls of the arteries of the heart, which narrows the lumen of arteries and reduces blood flow to the heart.

“Xanthoma” is a cutaneous manifestation of lipidosis in which lipids accumulate in large foam cells within the skin. Xanthomas are associated with hyperlipidemias.

The term “elevated Lp(a) concentration”, as used herein, refers to a serum Lp(a) concentration above 30 mg/dl (75 nmol/L). “Elevated serum Lp(a)” means a serum Lp(a) level greater than about 14 mg/dL. In certain embodiments, a patient is considered to exhibit elevated serum Lp(a) if the level of serum Lp(a) measured in the patient is greater than about 15 mg/dL, about 20 mg/dL, about 25 mg/dL, about 30 mg/dL, about 35 mg/dL, about 40 mg/dL, about 45 mg/dL, about 50 mg/dL, about 60 mg/dL, about 70 mg/dL, about 80 mg/dL, about 90 mg/dL, about 100 mg/dL, about 20 mg/dL, about 140 mg/dL, about 150 mg dL, about 180 mg/dL, or about 200 mg/dL The serum Lp(a) level can be measured in a patient post-prandial. In some embodiments, the Lp(a) level is measured after a period of time of fasting (e.g., after fasting for 8 hrs, 8 hrs, 10 hrs, 12 hrs or more). Exemplary methods for measuring serum Lp(a) in a patient include, but are not limited to, rate immunonephelometry, ELISA, nephelometry, immunoturbidimetry, and dissociation-enhanced lanthanide fluorescent immunoassay, although any clinically acceptable diagnostic method can be used in the context of the present disclosure.

By “elevated triglyceride levels” or “ETL” is meant any degree of triglyceride levels that is determined to be undesirable or is targeted for modulation.

“Sepsis” is a systemic reaction characterized by arterial hypotension, metabolic acidosis, decreased systemic vascular resistance, tachypnea, and organ dysfunction. Sepsis can result from septicemia (i.e., organisms, their metabolic end-products or toxins in the blood stream), including bacteremia (i.e., bacteria in the blood), as well as toxemia (i.e., toxins in the blood), including endotoxemia (i.e., endotoxin in the blood). The term “sepsis” also encompasses fungemia (i.e., fungi in the blood), viremia (i.e., viruses or virus particles in the blood), and parasitemia (i.e., helminthic or protozoan parasites in the blood). Thus, septicemia and septic shock (acute circulatory failure resulting from septicemia often associated with multiple organ failure and a high mortality rate) may be caused by a number of organisms.

### Specific Embodiments of Compounds of Formula (I)

The present disclosure relates to compounds or pharmaceutically acceptable salts, hydrates, solvates, stereoisomers, or tautomers thereof, capable of modulating PCSK9, which are useful for the treatment of diseases and disorders associated with modulation of a PCSK9 protein or enzyme. In another embodiment, the present disclosure relates to compounds or pharmaceutically acceptable salts, hydrates, solvates, prodrugs, stereoisomers, or tautomers thereof, capable of inhibiting PCSK9, which are useful for the treatment of diseases and disorders associated with inhibition of a PCSK9 protein or enzyme. The disclosure further relates to compounds, or pharmaceutically acceptable salts, hydrates, solvates, stereoisomers, or tautomers thereof, which are useful for inhibiting PCSK9.

In one embodiment, the compounds of Formula (I) have the structure of Formula (Ia):

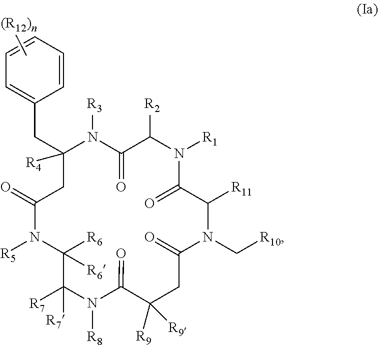

and pharmaceutically acceptable salts, hydrates, solvates, stereoisomers, and tautomers thereof.

In another embodiment, the compounds of Formula (I) have the structure of Formula (Ia-1):

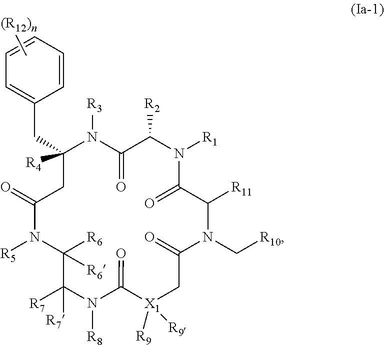

and pharmaceutically acceptable salts, hydrates, solvates, stereoisomers, and tautomers thereof.

In another embodiment, the compounds of Formula (I) have the structure of Formula (Ia-2):

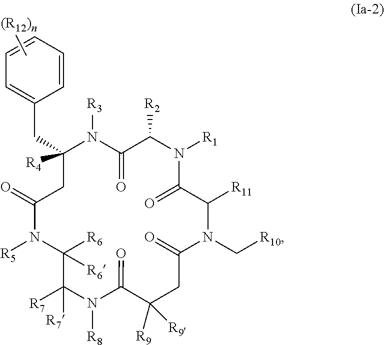

and pharmaceutically acceptable salts, hydrates, solvates, stereoisomers, and tautomers thereof.

In another embodiment, the compounds of Formula (I) have the structure of Formula (Ib):

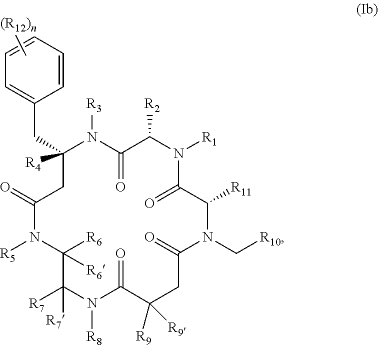

and pharmaceutically acceptable salts, hydrates, solvates, stereoisomers, and tautomers thereof.

In another embodiment, the compounds of Formula (I) have the structure of Formula (Ic):

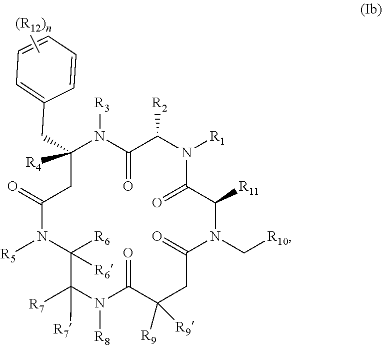

and pharmaceutically acceptable salts, hydrates, solvates, stereoisomers, and tautomers thereof.

In another embodiment, the compounds of Formula (I) have the structure of Formula (Id):

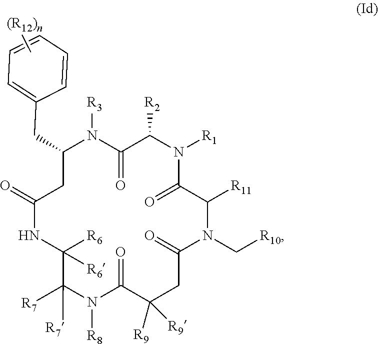

and pharmaceutically acceptable salts, hydrates, solvates, stereoisomers, and tautomers thereof.

In another embodiment, the compounds of Formula (I) have the structure of Formula (Ie):

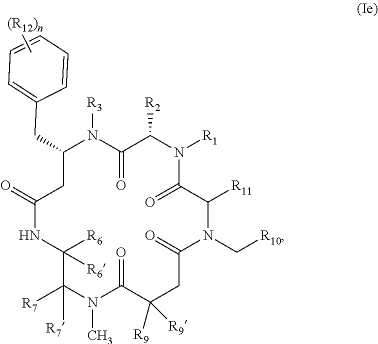

and pharmaceutically acceptable salts, hydrates, solvates, stereoisomers, and tautomers thereof.

In another embodiment, the compounds of Formula (I) have the structure of Formula (If):

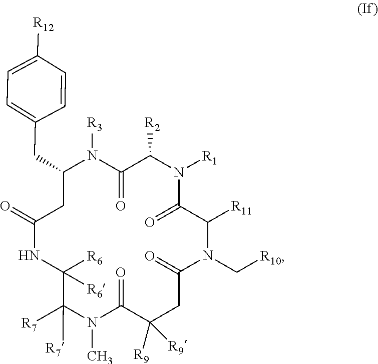

and pharmaceutically acceptable salts, hydrates, solvates, stereoisomers, and tautomers thereof.

In another embodiment, the compounds of Formula (I) have the structure of Formula (Ig):

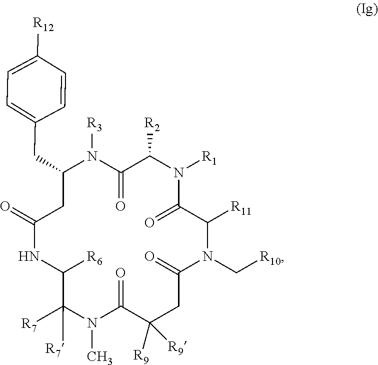

and pharmaceutically acceptable salts, hydrates, solvates, stereoisomers, and tautomers thereof.

In another embodiment, the compounds of Formula (I) have the structure of Formula (Ih):

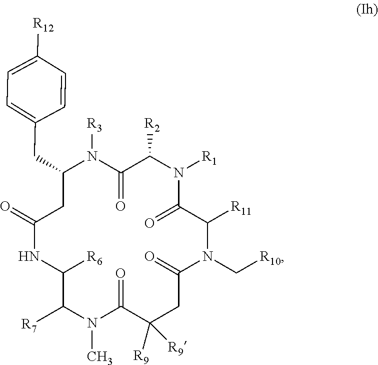

and pharmaceutically acceptable salts, hydrates, solvates, stereoisomers, and tautomers thereof.

In another embodiment, the compounds of Formula (I) have the structure of Formula (Ii):

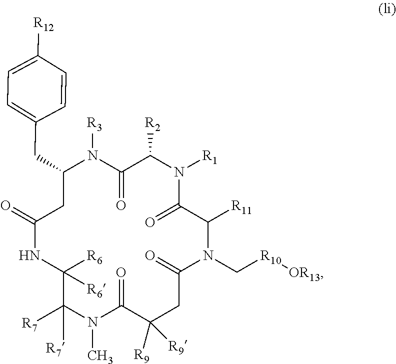

and pharmaceutically acceptable salts, hydrates, solvates, stereoisomers, and tautomers thereof.

In another embodiment, the compounds of Formula (I) have the structure of Formula (Ij):

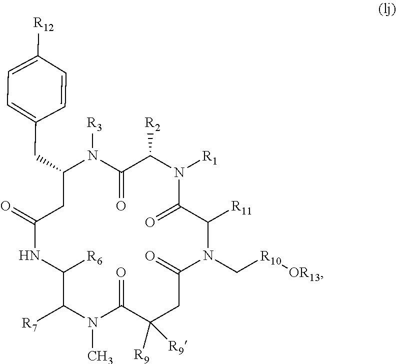

and pharmaceutically acceptable salts, hydrates, solvates, stereoisomers, and tautomers thereof.

In some embodiments of the Formulae above (e.g., Formula (I), (Ia), (Ia-1), (Ia-2), (Ib), (Ic), (Id), (le), (If), (Ig), (Ih), (Ii), and/or (Ij)),

X₁ is C or N;

R₁ is H, (C₁-C₆)alkyl, or (C₁-C₆)haloalkyl; or

R₁ and R₁₁ together with the atoms to which they are attached form a 5- to 7-membered heterocyclyl ring comprising 1-3 heteroatoms selected from N, O, and S optionally substituted with one to four substituents each independently selected from ═(O), (C₁-C₆)alkyl, and (C₁-C₆)haloalkyl;

R₂ is (C₁-C₆)alkyl, (C₁-C₆)haloalkyl, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one to four substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S;

R₃, R₄, and R₅ are each independently H, (C₁-C₆)alkyl, or (C₁-C₆)haloalkyl;

R₆, R_{6′}, R₇, and R_{7′} are each independently H, (C₁-C₆)alkyl, (C₁-C₆)haloalkyl, (C₁-C₆)hydroxyalkyl, —C(O)OH, or —C(O)O(C₁-C₆)alkyl, (C₁-C₆)hydroxyalkyl, wherein the alkyl is optionally substituted with one or more substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S; or

R₆ and R₇ together with the carbon atoms to which they are attached form a (C₃-C₇)cycloalkyl or a 4- to 7-membered heterocyclyl ring comprising 1-3 heteroatoms selected from N, O, and S; or

R₇ and R_{7′} together with the carbon atom to which they are attached form a (C₃-C₇)cycloalkyl or a 4- to 7-membered heterocyclyl ring comprising 1-3 heteroatoms selected from N, O, and S; or

R₇ and R₉ together with the atoms to which they are attached form a 5- to 7-membered heterocyclyl ring comprising 1-3 heteroatoms selected from N, O, and S optionally substituted with one to four substituents independently selected from (C₁-C₆)alkyl, (C₁-C₆)haloalkyl, and ═(O);

R₈ is H or (C₁-C₆)alkyl;

R₉ and R_{9′} are each independently H, (C₁-C₆)alkyl, (C₂-C₆)alkenyl, (C₁-C₆)haloalkyl, (C₂-C₆)haloalkenyl, (C₁-C₆)alkoxy, (C₁-C₆)haloalkoxy, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one to four R₂₇; or R_{9′} is absent when X₁ is N; or

R₉ and R_{9′} together with the carbon atom to which they are attached form a (C₃-C₇)cycloalkyl or a 4- to 7-membered heterocyclyl ring comprising 1-3 heteroatoms selected from N, O, and S; or

R₇ and R₉ together with the atoms to which they are attached form a 5- to 7-membered heterocyclyl ring comprising 1-3 heteroatoms selected from N, O, and S optionally substituted with one to four substituents independently selected from (C₁-C₆)alkyl, (C₁-C₆)haloalkyl, and ═(O);

R₁₀ is (C₆-C₁₀)aryl, 5- or 6-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the cycloalkyl, heterocyclyl, aryl and heteroaryl are substituted with —OR₁₃ or —NR₂₃R₁₃, and optionally substituted with one to four R₁₄;

R₁₁ is (C₁-C₆)alkyl, (C₁-C₆)haloalkyl, or (C₁-C₆)hydroxyalkyl, wherein the alkyl is optionally substituted with one to four R₁₅; or

R₁ and R₁₁ together with the atoms to which they are attached form a 5- to 7-membered heterocyclyl ring comprising 1-3 heteroatoms selected from N, O, and S optionally substituted with one to four substituents each independently selected from ═(O), (C₁-C₆)alkyl, and (C₁-C₆)haloalkyl;

R₁₂ is halogen, (C₁-C₆)alkyl, (C₁-C₆)alkoxy, (C₁-C₆)haloalkyl, (C₁-C₆)haloalkoxy, —OH, or CN;

R₁₃ is (C₆-C₁₀)aryl, or 5- or 6-membered heteroaryl comprising 1-3 heteroatoms selected from N, O, and S, wherein the aryl and heteroaryl are substituted with R₁₆ and optionally substituted with one to four R_{16′};

each R₁₄ is independently at each occurrence halogen, (C₁-C₆)alkyl, (C₁-C₆)alkoxy, (C₁-C₆)haloalkyl, (C₁-C₆)haloalkoxy, oxo, —OH, or CN; or

when R₁₀ is cycloalkyl or heterocyclyl, two R₁₄ together, when attached to the same carbon atom, together form ═(O);

each R₁₅ is independently at each occurrence (C₁-C₆)alkoxy, (C₁-C₆)haloalkoxy, —C(O)R₁₉, —S(O)_{q}(C₁-C₆)alkyl, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, —NR₁₇C(O)OR₁₈, (C₃-C₇)cycloalkyl, or 4- to 7-membered heterocyclyl comprising 1-3 heteroatoms selected from N, O, and S, wherein the cycloalkyl and heterocyclyl are optionally substituted with one to four R₂₁;

R₁₆ is —C(O)NR₃₁R₃₂, (C₆-C₁₀)aryl, or 5- to 7-membered heteroaryl comprising 1-3 heteroatoms selected from N, O, and S, wherein the aryl and heteroaryl are optionally substituted with one to four R₂₆;

each R_{16′} is independently at each occurrence halogen, (C₁-C₆)alkyl, (C₁-C₆)alkoxy, (C₁-C₆)haloalkyl, (C₁-C₆)haloalkoxy, —OH, or CN; or

R₁₆ and R_{16′} together with the atoms to which they are attached form a 5- to 7-membered heterocyclyl ring optionally substituted with one to four substituents independently selected from ═(O) and R₃₄;

R₁₇ and R₁₈ are each independently H or (C₁-C₆)alkyl optionally substituted with one to four substituents each independently selected from (C₁-C₆)alkoxy and —C(O)O(C₁-C₆)alkyl;

R₁₉ is (C₃-C₇)cycloalkyl or 4- to 7-membered heterocyclyl comprising 1-3 heteroatoms selected from N, O, and S, optionally substituted with one to four R₂₂;

R₂₀ is —(CH₂CH₂O)ₘCH₂CH₂ONH₂, —(CH₂CH₂O)ₘCH₂CH₂ONH(C₁-C₆)alkyl, or (C₁-C₆)alkyl optionally substituted with one to four —NR₂₃C(O)R₂₄;

each R₂₁ is independently at each occurrence (C₁-C₆)alkyl, (C₁-C₆)alkoxy, (C₁-C₆)haloalkyl, (C₁-C₆)haloalkoxy, halogen, ═(O), or —OH;

each R₂₂ is independently at each occurrence (C₁-C₆)alkyl, (C₁-C₆)haloalkyl, halogen, or —OH; or

two R₂₂, when on the same atom, together with the atom to which they are attached form a (C₃-C₇)spirocycloalkyl or a 4- to 7-membered spiroheterocyclyl ring comprising 1-3 heteroatoms selected from N, O, and S;

R₂₃ is H or (C₁-C₆)alkyl;

R₂₄ is H or (C₁-C₆)alkyl optionally substituted with one to four R₂₅;

each R₂₅ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with one to four substituents independently selected from (C₁-C₆)alkyl, (C₁-C₆)haloalkyl, and ═(O);

each R₂₆ is independently at each occurrence (C₁-C₆)alkyl optionally substituted with one to four R₂₉, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one to four substituents each independently selected from (C₁-C₆)alkyl, (C₁-C₆)haloalkyl, —NH₂, —N(H)(C₁-C₆)alkyl, —N((C₁-C₆)alkyl)₂, —N(H)(C₁-C₆)haloalkyl, —N((C₁-C₆)haloalkyl)₂, halogen, and —OH; or

two R₂₆, when on adjacent atoms, together with the atoms to which they are attached form a (C₃-C₇)carbocyclyl or a 4- to 7-membered heterocyclyl ring comprising 1-3 heteroatoms selected from N, O, and S, optionally substituted with one to four R₃₃;

each R₂₇ is independently at each occurrence CN, (C₆-C₁₀)aryl, 5- to 7-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl and heterocyclyl are optionally substituted with one to four R₂₈;

each R₂₈ is independently at each occurrence (C₁-C₆)alkyl, (C₁-C₆)haloalkyl, (C₁-C₆)alkoxy, (C₁-C₆)haloalkoxy, (C₁-C₆)hydroxyalkyl, halogen, oxo, or CN; or

when R₂₇ is cycloalkyl or heterocyclyl, two R₂₈ together with the atoms to which they are attached form a (C₄-C₇)cycloalkyl or a 4- to 7-membered heterocyclyl ring comprising 1-3 heteroatoms selected from N, O, and S; or

when R₂₇ is cycloalkyl or heterocyclyl, two R₂₈ together, when attached to the same carbon atom, together form ═(O);

each R₂₉ is independently at each occurrence —NR₃₁R₃₂ or 4- to 7-membered heterocyclyl comprising 1-3 heteroatoms selected from N, O, and S optionally substituted with one to four R₃₀;

each R₃₀ is independently at each occurrence —OH, halogen, (C₁-C₆)alkyl, or (C₁-C₆)haloalkyl; or

two R₃₀, when on the same atom, together with the atom to which they are attached form a (C₃-C₇)spirocycloalkyl or 4- to 7-membered spiroheterocyclyl ring comprising 1-3 heteroatoms selected from N, O, and S;

R₃₁ and R₃₂ are each independently H, (C₁-C₆)alkyl, (C₁-C₆)hydroxyalkyl, (C₃-C₇) cycloalkyl, or 4- to 7-membered heterocyclyl comprising 1-3 heteroatoms selected from N, O, and S, wherein the alkyl is optionally substituted with one to six D, and the cycloalkyl and heterocyclyl are optionally substituted with one to four substituents each independently selected from (C₁-C₆)alkyl, (C₁-C₆)haloalkyl, halogen, and —OH;

each R₃₃ is independently at each occurrence (C₁-C₆)alkyl, (C₁-C₆)haloalkyl, or —C(O)R, wherein R is (C₁-C₆)haloalkyl, (C₃-C₇)cycloalkyl, 4- to 7-membered heterocyclyl comprising 1-3 heteroatoms selected from N, O, and S, or (C₁-C₆)alkyl optionally substituted with one to four (C₁-C₆)alkoxy;

two R₃₃, when on the same atom, together with the atom to which they are attached form a (C₃-C₇)spirocycloalkyl or a 4- to 7-membered spiroheterocyclyl ring comprising 1-3 heteroatoms selected from N, O, and S;

each R₃₄ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with (C₁-C₆)alkyl optionally substituted with one to four substituents each independently selected from (C₃-C₇)cycloalkyl and 4- to 10-membered mono or bicyclic heterocyclyl comprising 1-4 heteroatoms selected from N, O, and S; m is 1-13;

n is 1, 2, 3, or 4; and

q is 0, 1, or 2;

or pharmaceutically acceptable salts, hydrates, solvates, stereoisomers, N-oxides, or tautomers thereof.

In some embodiments of the Formulae above,

X₁ is C or N;

R₁ is H, (C₁-C₆)alkyl, or (C₁-C₆)haloalkyl; or

R₁ and R₁₁ together with the atoms to which they are attached form a 5- to 7-membered heterocyclyl ring comprising 1-3 heteroatoms selected from N, O, and S optionally substituted with one to four substituents each independently selected from ═(O), (C₁-C₆)alkyl, and (C₁-C₆)haloalkyl;

R₂ is (C₁-C₆)alkyl, (C₁-C₆)haloalkyl, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one to four substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S;

R₃, R₄, and R₅ are each independently H, (C₁-C₆)alkyl, or (C₁-C₆)haloalkyl;

R₆, R_{6′}, R₇, and R_{7′} are each independently H, (C₁-C₆)alkyl, (C₁-C₆)haloalkyl, or (C₁-C₆)hydroxyalkyl, wherein the alkyl is optionally substituted with one to four substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S; or

R₆ and R₇ together with the carbon atoms to which they are attached form a (C₃-C₇)cycloalkyl or a 4- to 7-membered heterocyclyl ring comprising 1-3 heteroatoms selected from N, O, and S; or

R₇ and R_{7′} together with the carbon atom to which they are attached form a (C₃-C₇)cycloalkyl or a 4- to 7-membered heterocyclyl ring comprising 1-3 heteroatoms selected from N, O, and S; or

R₇ and R₉ together with the atoms to which they are attached form a 5- to 7-membered heterocyclyl ring comprising 1-3 heteroatoms selected from N, O, and S optionally substituted with one to four substituents independently selected from (C₁-C₆)alkyl, (C₁-C₆)haloalkyl, and ═(O);

R₈ is H or (C₁-C₆)alkyl;

R₉ and R_{9′} are each independently H, (C₁-C₆)alkyl, (C₂-C₆)alkenyl, (C₁-C₆)haloalkyl, (C₂-C₆)haloalkenyl, (C₁-C₆)alkoxy, (C₁-C₆)haloalkoxy, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one to four R₂₇; or R_{9′} is absent when X₁ is N; or

R₉ and R_{9′} together with the carbon atom to which they are attached form a (C₃-C₇)cycloalkyl or a 4- to 7-membered heterocyclyl ring comprising 1-3 heteroatoms selected from N, O, and S; or

R₇ and R₉ together with the atoms to which they are attached form a 5- to 7-membered heterocyclyl ring comprising 1-3 heteroatoms selected from N, O, and S optionally substituted with one to four substituents independently selected from (C₁-C₆)alkyl, (C₁-C₆)haloalkyl, and ═(O);

R₁₀ is (C₆-C₁₀)aryl, 5- or 6-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the cycloalkyl, heterocyclyl, aryl and heteroaryl are substituted with —OR₁₃ or —NR₂₃R₁₃, and optionally substituted with one to four R₁₄;

R₁₁ is (C₁-C₆)alkyl, (C₁-C₆)haloalkyl, or (C₁-C₆)hydroxyalkyl, wherein the alkyl is optionally substituted with one to four R₁₅; or

R₁ and R₁₁ together with the atoms to which they are attached form a 5- to 7-membered heterocyclyl ring comprising 1-3 heteroatoms selected from N, O, and S optionally substituted with one to four substituents each independently selected from ═(O), (C₁-C₆)alkyl, and (C₁-C₆)haloalkyl;

R₁₂ is halogen, (C₁-C₆)alkyl, (C₁-C₆)alkoxy, (C₁-C₆)haloalkyl, (C₁-C₆)haloalkoxy, —OH, or CN;

R₁₃ is (C₆-C₁₀)aryl, or 5- or 6-membered heteroaryl comprising 1-3 heteroatoms selected from N, O, and S, wherein the aryl and heteroaryl are substituted with R₁₆ and optionally substituted with one to four R_{16′};

each R₁₄ is independently at each occurrence halogen, (C₁-C₆)alkyl, (C₁-C₆)alkoxy, (C₁-C₆)haloalkyl, (C₁-C₆)haloalkoxy, oxo, —OH, or CN; or

when R₁₀ is cycloalkyl or heterocyclyl, two R₁₄ together, when attached to the same carbon atom, together form ═(O);

each R₁₅ is independently at each occurrence (C₁-C₆)alkoxy, (C₁-C₆)haloalkoxy, —C(O)R₁₉, —S(O)_{q}(C₁-C₆)alkyl, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, —NR₁₇C(O)OR₁₈, (C₃-C₇)cycloalkyl, or 4- to 7-membered heterocyclyl comprising 1-3 heteroatoms selected from N, O, and S, wherein the cycloalkyl and heterocyclyl are optionally substituted with one to four R₂₁;

R₁₆ s is —C(O)NR₃₁R₃₂, (C₆-C₁₀)aryl, or 5- to 7-membered heteroaryl comprising 1-3 heteroatoms selected from N, O, and S, wherein the aryl and heteroaryl are optionally substituted with one to four R₂₆;

each R_{16′} is independently at each occurrence halogen, (C₁-C₆)alkyl, (C₁-C₆)alkoxy, (C₁-C₆)haloalkyl, (C₁-C₆)haloalkoxy, —OH, or CN; or

R₁₆ and R_{16′} together with the atoms to which they are attached form a 5- to 7-membered heterocyclyl ring optionally substituted with one to four substituents independently selected from ═(O) and R₃₄;

R₁₇ and R₁₈ are each independently H or (C₁-C₆)alkyl optionally substituted with one to four substituents each independently selected from (C₁-C₆)alkoxy and —C(O)O(C₁-C₆)alkyl;

R₁₉ is (C₃-C₇)cycloalkyl or 4- to 7-membered heterocyclyl comprising 1-3 heteroatoms selected from N, O, and S, optionally substituted with one to four R₂₂;

R₂₀ is —(CH₂CH₂O)ₘCH₂CH₂ONH₂, —(CH₂CH₂O)ₘCH₂CH₂ONH(C₁-C₆)alkyl, or (C₁-C₆)alkyl optionally substituted with one to four —NR₂₃C(O)R₂₄;

each R₂₁ is independently at each occurrence (C₁-C₆)alkyl, (C₁-C₆)alkoxy, (C₁-C₆)haloalkyl, (C₁-C₆)haloalkoxy, halogen, ═(O), or —OH;

each R₂₂ is independently at each occurrence (C₁-C₆)alkyl, (C₁-C₆)haloalkyl, halogen, or —OH; or

two R₂₂, when on the same atom, together with the atom to which they are attached form a (C₃-C₇)spirocycloalkyl or a 4- to 7-membered spiroheterocyclyl ring comprising 1-3 heteroatoms selected from N, O, and S;

R₂₃ is H or (C₁-C₆)alkyl;

R₂₄ is H or (C₁-C₆)alkyl optionally substituted with one to four R₂₅;

each R₂₅ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with one to four substituents independently selected from (C₁-C₆)alkyl, (C₁-C₆)haloalkyl, and ═(O);

each R₂₆ is independently at each occurrence (C₁-C₆)alkyl optionally substituted with one to four R₂₉, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one to four substituents each independently selected from (C₁-C₆)alkyl, (C₁-C₆)haloalkyl, —NH₂, —N(H)(C₁-C₆)alkyl, —N((C₁-C₆)alkyl)₂, —N(H)(C₁-C₆)haloalkyl, —N((C₁-C₆)haloalkyl)₂, halogen, and —OH; or

two R₂₆, when on adjacent atoms, together with the atoms to which they are attached form a (C₃-C₇)carbocyclyl or a 4- to 7-membered heterocyclyl ring comprising 1-3 heteroatoms selected from N, O, and S, optionally substituted with one to four R₃₃;

each R₂₇ is independently at each occurrence CN, (C₆-C₁₀)aryl, 5- to 7-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl and heterocyclyl are optionally substituted with one to four R₂₈;

each R₂₈ is independently at each occurrence (C₁-C₆)alkyl, (C₁-C₆)haloalkyl, (C₁-C₆)alkoxy, (C₁-C₆)haloalkoxy, (C₁-C₆)hydroxyalkyl, halogen, oxo, or CN; or

when R₂₇ is cycloalkyl or heterocyclyl, two R₂₈ together with the atoms to which they are attached form a (C₄-C₇)cycloalkyl or a 4- to 7-membered heterocyclyl ring comprising 1-3 heteroatoms selected from N, O, and S; or

when R₂₇ is cycloalkyl or heterocyclyl, two R₂₈ together, when attached to the same carbon atom, together form ═(O);

each R₂₉ is independently at each occurrence —NR₃₁R₃₂ or 4- to 7-membered heterocyclyl comprising 1-3 heteroatoms selected from N, O, and S optionally substituted with one to four R₃₀;

each R₃₀ is independently at each occurrence —OH, halogen, (C₁-C₆)alkyl, or (C₁-C₆)haloalkyl; or

two R₃₀, when on the same atom, together with the atom to which they are attached form a (C₃-C₇)spirocycloalkyl or 4- to 7-membered spiroheterocyclyl ring comprising 1-3 heteroatoms selected from N, O, and S;

R₃₁ and R₃₂ are each independently H, (C₁-C₆)alkyl, (C₁-C₆)hydroxyalkyl, (C₃-C₇) cycloalkyl, or 4- to 7-membered heterocyclyl comprising 1-3 heteroatoms selected from N, O, and S, wherein the alkyl is optionally substituted with one to six D, and the cycloalkyl and heterocyclyl are optionally substituted with one to four substituents each independently selected from (C₁-C₆)alkyl, (C₁-C₆)haloalkyl, halogen, and —OH;

each R₃₃ is independently at each occurrence (C₁-C₆)alkyl, (C₁-C₆)haloalkyl, or —C(O)R, wherein R is (C₁-C₆)haloalkyl, (C₃-C₇)cycloalkyl, 4- to 7-membered heterocyclyl comprising 1-3 heteroatoms selected from N, O, and S, or (C₁-C₆)alkyl optionally substituted with one to four (C₁-C₆)alkoxy;

two R₃₃, when on the same atom, together with the atom to which they are attached form a (C₃-C₇)spirocycloalkyl or a 4- to 7-membered spiroheterocyclyl ring comprising 1-3 heteroatoms selected from N, O, and S;

each R₃₄ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with (C₁-C₆)alkyl optionally substituted with one to four substituents each independently selected from (C₃-C₇)cycloalkyl and 4- to 10-membered mono or bicyclic heterocyclyl comprising 1-4 heteroatoms selected from N, O, and S; m is 1-13;

n is 1, 2, 3, or 4; and

q is 0, 1, or 2;

or pharmaceutically acceptable salts, hydrates, solvates, stereoisomers, N-oxides, or tautomers thereof.

In some embodiments of the Formulae above, R₁ is H, (C₁-C₃)alkyl, or (C₁-C₃)haloalkyl.

In another embodiment, R₁ is (C₁-C₃)alkyl or (C₁-C₃)haloalkyl. In yet another embodiment, R₁ is H or (C₁-C₃)alkyl. In another embodiment, R₁ is H, methyl, ethyl, n-propyl or i-propyl. In yet another embodiment, R₁ is H, methyl, or ethyl. In another embodiment, R₁ is H or methyl. In yet another embodiment, R₁ is H.

In some embodiments of the Formulae above, R₁ and R₁₁ together with the atoms to which they are attached form a 5- to 7-membered heterocyclyl ring comprising 1-3 heteroatoms selected from N, O, and S optionally substituted with one to three substituents each independently selected from ═(O), (C₁-C₆)alkyl, and (C₁-C₆)haloalkyl. In another embodiment, R₁ and R₁₁ together with the atoms to which they are attached form a 5- to 7-membered heterocyclyl ring comprising 1-3 heteroatoms selected from N, O, and S substituted with one to three substituents each independently selected from ═(O), (C₁-C₆)alkyl, and (C₁-C₆)haloalkyl. In yet another embodiment, R₁ and R₁₁ together with the atoms to which they are attached form a 5- or 6-membered heterocyclyl ring comprising 1-3 heteroatoms selected from N, O, and S optionally substituted with one to three substituents each independently selected from ═(O), (C₁-C₆)alkyl, and (C₁-C₆)haloalkyl. In another embodiment, R₁ and R₁₁ together with the atoms to which they are attached form a 6- or 7-membered heterocyclyl ring comprising 1-3 heteroatoms selected from N, O, and S optionally substituted with one to three substituents each independently selected from ═(O), (C₁-C₆)alkyl, and (C₁-C₆)haloalkyl.

In some embodiments of the Formulae above, R₂ is (C₁-C₄)alkyl, (C₁-C₄)haloalkyl, (C₁-C₄)hydroxyalkyl, (C₃-C₇)cycloalkyl, or 4- to 7-membered heterocyclyl comprising 1-3 heteroatoms selected from N, O, and S, wherein the alkyl is optionally substituted with one to three substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S. In another embodiment, R₂ is (C₁-C₄)alkyl, (C₁-C₄)haloalkyl, or (C₁-C₄)hydroxyalkyl, wherein the alkyl is optionally substituted with one to three substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S.

In another embodiment, R₂ is (C₁-C₄)alkyl, (C₁-C₄)haloalkyl, (C₁-C₄)hydroxyalkyl, or 4- to 6-membered heterocyclyl comprising 1-3 heteroatoms selected from N, O, and S, wherein the alkyl is optionally substituted with one to three substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S. In yet another embodiment, R₂ is (C₁-C₃)alkyl, (C₁-C₃)haloalkyl, (C₁-C₃)hydroxyalkyl, or 4- to 6-membered heterocyclyl comprising 1-3 heteroatoms selected from N, O, and S, wherein the alkyl is optionally substituted with one to three substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S.

In another embodiment, R₂ is (C₁-C₃)alkyl, (C₁-C₃)haloalkyl, (C₁-C₃)hydroxyalkyl, or 4- to 6-membered heterocyclyl comprising 1-3 heteroatoms selected from N, O, and S, wherein the alkyl is optionally substituted with one to two substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S. In yet another embodiment, R₂ is (C₁-C₃)alkyl, (C₁-C₃)haloalkyl, (C₁-C₃)hydroxyalkyl, or 4- to 6-membered heterocyclyl comprising 1-3 heteroatoms selected from N, O, and S, wherein the alkyl is optionally substituted with one to two substituents each independently selected from (C₁-C₃)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S.

In another embodiment, R₂ is (C₁-C₃)alkyl, (C₁-C₃)haloalkyl, (C₁-C₃)hydroxyalkyl, or 4- to 6-membered heterocyclyl comprising 1-3 heteroatoms selected from N, O, and S, wherein the alkyl is optionally substituted with one to two substituents each independently selected from (C₁-C₃)alkoxy, (C₁-C₆)haloalkoxy, —C(O)(C₁-C₆)alkyl, —C(O)OH, —OC(O)(C₁-C₆)alkyl, —C(O)NR₁₇R₁₈, and 5- or 6-membered heteroaryl comprising 1-4 heteroatoms selected from N, O, and S. In yet another embodiment, R₂ is (C₁-C₃)alkyl, (C₁-C₃)haloalkyl, or (C₁-C₃)hydroxyalkyl, wherein the alkyl is optionally substituted with one to two substituents each independently selected from (C₁-C₃)alkoxy, (C₁-C₆)haloalkoxy, —C(O)(C₁-C₆)alkyl, —C(O)OH, —OC(O)(C₁-C₆)alkyl, —C(O)NR₁₇R₁₈, and 5- or 6-membered heteroaryl comprising 1-4 heteroatoms selected from N, O, and S.

In some embodiments of the Formulae above, R₃ is H, (C₁-C₃)alkyl, or (C₁-C₃)haloalkyl. In another embodiment, R₃ is (C₁-C₃)alkyl or (C₁-C₃)haloalkyl. In yet another embodiment, R₃ is H or (C₁-C₃)alkyl. In another embodiment, R₃ is H, methyl, ethyl, n-propyl or i-propyl. In yet another embodiment, R₃ is H, methyl, or ethyl. In another embodiment, R₃ is H or methyl.

In some embodiments of the Formulae above, R₄ is H, (C₁-C₃)alkyl, or (C₁-C₃)haloalkyl. In another embodiment, R₄ is (C₁-C₃)alkyl or (C₁-C₃)haloalkyl. In yet another embodiment, R₄ is H or (C₁-C₃)alkyl. In another embodiment, R₄ is H, methyl, ethyl, n-propyl or i-propyl. In yet another embodiment, R₄ is H, methyl, or ethyl. In another embodiment, R₄ is H or methyl. In yet another embodiment, R₄ is H.

In some embodiments of the Formulae above, R₅ is H, (C₁-C₃)alkyl, or (C₁-C₃)haloalkyl. In another embodiment, R₅ is (C₁-C₃)alkyl or (C₁-C₃)haloalkyl. In yet another embodiment, R₅ is H or (C₁-C₃)alkyl. In another embodiment, R₅ is H, methyl, ethyl, n-propyl or i-propyl. In yet another embodiment, R₅ is H, methyl, or ethyl. In another embodiment, R₅ is H or methyl. In yet another embodiment, R₅ is H.

In some embodiments of the Formulae above, R₆ is H, (C₁-C₃)alkyl, (C₁-C₃)haloalkyl, or (C₁-C₃)hydroxyalkyl, wherein the alkyl is optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In another embodiment, R₆ is H, (C₁-C₃)alkyl, (C₁-C₃)haloalkyl, or (C₁-C₃)hydroxyalkyl, wherein the alkyl is substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R₆ is H, (C₁-C₃)alkyl, (C₁-C₃)haloalkyl, or (C₁-C₃)hydroxyalkyl. In another embodiment, R₆ is H, (C₁-C₃)alkyl, or (C₁-C₃)hydroxyalkyl, wherein the alkyl is optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S.

In another embodiment, R₆ is H or (C₁-C₃)alkyl optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R₆ is H or (C₁-C₃)alkyl substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In another embodiment, R₆ is H or (C₁-C₃)alkyl. In yet another embodiment, R₆ is H, methyl, ethyl, n-propyl or i-propyl. In another embodiment, R₆ is H, methyl, or ethyl. In yet another embodiment, R₆ is H or methyl.

In some embodiments of the Formulae above, R_{6′} is H, (C₁-C₃)alkyl, (C₁-C₃)haloalkyl, or (C₁-C₃)hydroxyalkyl, wherein the alkyl is optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In another embodiment, R_{6′} is H, (C₁-C₃)alkyl, (C₁-C₃)haloalkyl, or (C₁-C₃)hydroxyalkyl, wherein the alkyl is substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R_{6′} is H, (C₁-C₃)alkyl, (C₁-C₃)haloalkyl, or (C₁-C₃)hydroxyalkyl. In another embodiment, R_{6′} is H, (C₁-C₃)alkyl, or (C₁-C₃)hydroxyalkyl, wherein the alkyl is optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S.

In another embodiment, R_{6′} is H or (C₁-C₃)alkyl optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R_{6′} is H or (C₁-C₃)alkyl substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In another embodiment, R_{6′} is H or (C₁-C₃)alkyl. In yet another embodiment, R_{6′} is H, methyl, ethyl, n-propyl or i-propyl. In another embodiment, R_{6′} is H, methyl, or ethyl. In yet another embodiment, R_{6′} is H or methyl.

In some embodiments of the Formulae above, R₇ is H, (C₁-C₃)alkyl, (C₁-C₃)haloalkyl, or (C₁-C₃)hydroxyalkyl, wherein the alkyl is optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In another embodiment, R₇ is H, (C₁-C₃)alkyl, (C₁-C₃)haloalkyl, or (C₁-C₃)hydroxyalkyl, wherein the alkyl is substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R₇ is H, (C₁-C₃)alkyl, (C₁-C₃)haloalkyl, or (C₁-C₃)hydroxyalkyl. In another embodiment, R₇ is H, (C₁-C₃)alkyl, or (C₁-C₃)hydroxyalkyl, wherein the alkyl is optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S.

In another embodiment, R₇ is H or (C₁-C₃)alkyl optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R₇ is H or (C₁-C₃)alkyl substituted with one to three each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In another embodiment, R₇ is H or (C₁-C₃)alkyl. In yet another embodiment, R₇ is H, methyl, ethyl, n-propyl or i-propyl. In another embodiment, R₇ is H, methyl, or ethyl. In yet another embodiment, R₇ is H or methyl. In another embodiment, R₇ is H.

In some embodiments of the Formulae above, R_{7′} is H, (C₁-C₃)alkyl, (C₁-C₃)haloalkyl, or (C₁-C₃)hydroxyalkyl, wherein the alkyl is optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In another embodiment, R_{7′} is H, (C₁-C₃)alkyl, (C₁-C₃)haloalkyl, or (C₁-C₃)hydroxyalkyl, wherein the alkyl is substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R_{7′} is H, (C₁-C₃)alkyl, (C₁-C₃)haloalkyl, or (C₁-C₃)hydroxyalkyl. In another embodiment, R_{7′} is H, (C₁-C₃)alkyl, or (C₁-C₃)hydroxyalkyl, wherein the alkyl is optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S.

In another embodiment, R_{7′} is H or (C₁-C₃)alkyl optionally substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R_{7′} is H or (C₁-C₃)alkyl substituted with one to three substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S. In yet another embodiment, R_{7′} is H, (C₁-C₃)alkyl, (C₁-C₃)haloalkyl, or (C₁-C₃)hydroxyalkyl, wherein the alkyl is optionally substituted with one to three substituents each independently selected from (C₁-C₃)alkoxy, —C(O)OH, —C(O)O(C₁-C₃)alkyl, —NR₁₇R₁₈, —NR₁₇C(O)R₁₈, and (C₃-C₇)cycloalkyl.

In some embodiments of the Formulae above, R₆ and R₇ together with the carbon atoms to which they are attached form a (C₃-C₇)cycloalkyl or a 4- to 7-membered heterocyclyl ring comprising 1-3 heteroatoms selected from N, O, and S. In another embodiment, R₆ and R₇ together with the carbon atoms to which they are attached form a (C₃-C₇)cycloalkyl ring. In yet another embodiment, R₆ and R₇ together with the carbon atoms to which they are attached form a (C₄-C₇)cycloalkyl ring. In another embodiment, R₆ and R₇ together with the carbon atoms to which they are attached form a (C₅-C₇)cycloalkyl ring. In yet another embodiment, R₆ and R₇ together with the carbon atoms to which they are attached form a (C₆-C₇)cycloalkyl ring. In another embodiment, R₆ and R₇ together with the carbon atoms to which they are attached form a (C₅-C₆)cycloalkyl ring. In yet another embodiment, R₆ and R₇ together with the carbon atoms to which they are attached form a (C₄-C₆)cycloalkyl ring.

In another embodiment, R₆ and R₇ together with the carbon atoms to which they are attached form a 4- to 7-membered heterocyclyl ring comprising 1-3 heteroatoms selected from N, O, and S. In yet another embodiment, R₆ and R₇ together with the carbon atoms to which they are attached form a 5- to 7-membered heterocyclyl ring comprising 1-3 heteroatoms selected from N, O, and S. In another embodiment, R₆ and R₇ together with the carbon atoms to which they are attached form a 6- to 7-membered heterocyclyl ring comprising 1-3 heteroatoms selected from N, O, and S. In yet another embodiment, R₆ and R₇ together with the carbon atoms to which they are attached form a 4- to 6-membered heterocyclyl ring comprising 1-3 heteroatoms selected from N, O, and S. In another embodiment, R₆ and R₇ together with the carbon atoms to which they are attached form a 5- or 6-membered heterocyclyl ring comprising 1-3 heteroatoms selected from N, O, and S.

In some embodiments of the Formulae above, R₇ and R_{7′} together with the carbon atom to which they are attached form a (C₃-C₇)cycloalkyl or a 4- to 7-membered heterocyclyl ring comprising 1-3 heteroatoms selected from N, O, and S. In another embodiment, R₇ and R_{7′} together with the carbon atom to which they are attached form a (C₃-C₇)cycloalkyl ring. In another embodiment, R₇ and R_{7′} together with the carbon atom to which they are attached form a (C₄-C₇)cycloalkyl ring. In another embodiment, R₇ and R_{7′} together with the carbon atom to which they are attached form a (C₅-C₇)cycloalkyl ring. In another embodiment, R₇ and R_{7′} together with the carbon atom to which they are attached form a (C₆-C₇)cycloalkyl ring. In another embodiment, R₇ and R_{7′} together with the carbon atom to which they are attached form a (C₃-C₆)cycloalkyl ring. In another embodiment, R₇ and R_{7′} together with the carbon atom to which they are attached form a (C₃-C₅)cycloalkyl ring. In another embodiment, R₇ and R_{7′} together with the carbon atom to which they are attached form a (C₃-C₄)cycloalkyl ring.

In another embodiment, R₇ and R_{7′} together with the carbon atom to which they are attached form a 4- to 7-membered heterocyclyl ring comprising 1-3 heteroatoms selected from N, O, and S. In another embodiment, R₇ and R_{7′} together with the carbon atom to which they are attached form a 5- to 7-membered heterocyclyl ring comprising 1-3 heteroatoms selected from N, O, and S. In another embodiment, R₇ and R_{7′} together with the carbon atom to which they are attached form a 6- or 7-membered heterocyclyl ring comprising 1-3 heteroatoms selected from N, O, and S. In another embodiment, R₇ and R_{7′} together with the carbon atom to which they are attached form a 4- or 5-membered heterocyclyl ring comprising 1-3 heteroatoms selected from N, O, and S. In another embodiment, R₇ and R_{7′} together with the carbon atom to which they are attached form a 5- or 6-membered heterocyclyl ring comprising 1-3 heteroatoms selected from N, O, and S.

In another embodiment, R₇ and R_{7′} together with the carbon atom to which they are attached form a (C₃-C₆)cycloalkyl or a 4- to 6-membered heterocyclyl ring comprising 1-3 heteroatoms selected from N, O, and S. In another embodiment, R₇ and R_{7′} together with the carbon atom to which they are attached form a (C₃-C₆)cycloalkyl or a 5- or 6-membered heterocyclyl ring comprising 1-3 heteroatoms selected from N, O, and S.

In some embodiments of the Formulae above, R₇ and R₉ together with the atoms to which they are attached form a 5- to 7-membered heterocyclyl ring comprising 1-3 heteroatoms selected from N, O, and S optionally substituted with one or more substituents independently selected from (C₁-C₆)alkyl, (C₁-C₆)haloalkyl, and ═(O). In another embodiment, R₇ and R₉ together with the atoms to which they are attached form a 5- to 7-membered heterocyclyl ring comprising 1-3 heteroatoms selected from N, O, and S optionally substituted with one or more substituents independently selected from (C₁-C₆)alkyl and ═(O). In yet another embodiment, R₇ and R₉ together with the atoms to which they are attached form a 5- or 6-membered heterocyclyl ring comprising 1-3 heteroatoms selected from N, O, and S optionally substituted with one or more substituents independently selected from (C₁-C₆)alkyl, (C₁-C₆)haloalkyl, and ═(O).

In another embodiment, R₇ and R₉ together with the atoms to which they are attached form a 6- or 7-membered heterocyclyl ring comprising 1-3 heteroatoms selected from N, O, and S optionally substituted with one or more substituents independently selected from (C₁-C₆)alkyl, (C₁-C₆)haloalkyl, and ═(O). In yet another embodiment, R₇ and R₉ together with the atoms to which they are attached form a 5- or 6-membered heterocyclyl ring comprising 1-3 heteroatoms selected from N, O, and S optionally substituted with one or more substituents independently selected from (C₁-C₆)alkyl and ═(O). In another embodiment, R₇ and R₉ together with the atoms to which they are attached form a 6- or 7-membered heterocyclyl ring comprising 1-3 heteroatoms selected from N, O, and S optionally substituted with one or more substituents independently selected from (C₁-C₆)alkyl and ═(O).

In some embodiments of the Formulae above, R₈ is H or (C₁-C₃)alkyl. In another embodiment, R₈ is H, methyl, ethyl, n-propyl or i-propyl. In yet another embodiment, R₈ is H, methyl, or ethyl. In yet another embodiment, R₈ is H or methyl. In another embodiment, R₈ is methyl.

In some embodiments of the Formulae above, R₉ is H, (C₁-C₄)alkyl, (C₂-C₄)alkenyl, (C₁-C₄)haloalkyl, (C₂-C₄)haloalkenyl, (C₁-C₄)alkoxy, (C₁-C₄)haloalkoxy, (C₁-C₄)hydroxyalkyl, (C₃-C₇)cycloalkyl, or 4- to 7-membered heterocyclyl comprising 1-3 heteroatoms selected from N, O, and S, wherein the alkyl is optionally substituted with one to three R₂₇. In another embodiment, R₉ is H, (C₁-C₄)alkyl, (C₂-C₄)alkenyl, (C₁-C₄)haloalkyl, (C₂-C₄)haloalkenyl, (C₁-C₄)alkoxy, (C₁-C₄)hydroxyalkyl, (C₃-C₇)cycloalkyl, or 4- to 7-membered heterocyclyl comprising 1-3 heteroatoms selected from N, O, and S, wherein the alkyl is optionally substituted with one to three R₂₇.

In another embodiment, R₉ is H, (C₁-C₄)alkyl, (C₂-C₄)alkenyl, (C₂-C₄)haloalkenyl, (C₁-C₄)alkoxy, (C₁-C₄)hydroxyalkyl, (C₃-C₇)cycloalkyl, or 4- to 7-membered heterocyclyl comprising 1-3 heteroatoms selected from N, O, and S, wherein the alkyl is optionally substituted with one to three R₂₇. In yet another embodiment, R₉ is H, (C₁-C₄)alkyl, (C₂-C₄)alkenyl, (C₂-C₄)haloalkenyl, (C₁-C₄)alkoxy, (C₁-C₄)hydroxyalkyl, or (C₃-C₇)cycloalkyl, wherein the alkyl is optionally substituted with one to three R₂₇. In another embodiment, R₉ is H, (C₁-C₄)alkyl, (C₂-C₄)alkenyl, (C₂-C₄)haloalkenyl, (C₁-C₄)alkoxy, (C₁-C₄)hydroxyalkyl, or (C₃-C₇)cycloalkyl, wherein the alkyl is substituted with one to three R₂₇.

In another embodiment, R₉ is H, (C₁-C₄)alkyl, (C₂-C₄)alkenyl, (C₂-C₄)haloalkenyl, (C₁-C₄)alkoxy, or (C₃-C₇)cycloalkyl, wherein the alkyl is optionally substituted with one to three R₂₇. In yet another embodiment, R₉ is H, (C₁-C₄)alkyl, (C₂-C₄)alkenyl, (C₂-C₄)haloalkenyl, or (C₃-C₆)cycloalkyl, wherein the alkyl is optionally substituted with one to three R₂₇. In another embodiment, R₉ is H, (C₁-C₄)alkyl, (C₂-C₄)alkenyl, (C₂-C₄)haloalkenyl, or (C₃-C₆)cycloalkyl, wherein the alkyl is optionally substituted with one to three R₂₇. In yet another embodiment, R₉ is H, (C₁-C₄)alkyl, (C₂-C₄)alkenyl, or (C₃-C₆)cycloalkyl, wherein the alkyl is optionally substituted with one to three R₂₇. In another embodiment, R₉ is H, (C₁-C₄)alkyl, or (C₃-C₆)cycloalkyl, wherein the alkyl is optionally substituted with one to three R₂₇. In yet another embodiment, R₉ is H or (C₁-C₄)alkyl optionally substituted with one to three R₂₇. In another embodiment, R₉ is H or (C₁-C₄)alkyl substituted with one to three R₂₇.

In some embodiments of the Formulae above, R_{9′} is H, (C₁-C₄)alkyl, (C₂-C₄)alkenyl, (C₁-C₄)haloalkyl, (C₂-C₄)haloalkenyl, (C₁-C₄)alkoxy, (C₁-C₄)haloalkoxy, (C₁-C₄)hydroxyalkyl, (C₃-C₇)cycloalkyl, or 4- to 7-membered heterocyclyl comprising 1-3 heteroatoms selected from N, O, and S, wherein the alkyl is optionally substituted with one to three R₂₇. In another embodiment, R_{9′} is H, (C₁-C₄)alkyl, (C₂-C₄)alkenyl, (C₁-C₄)haloalkyl, (C₂-C₄)haloalkenyl, (C₁-C₄)alkoxy, (C₁-C₄)hydroxyalkyl, (C₃-C₇)cycloalkyl, or 4- to 7-membered heterocyclyl comprising 1-3 heteroatoms selected from N, O, and S, wherein the alkyl is optionally substituted with one to three R₂₇.

In another embodiment, R_{9′} is H, (C₁-C₄)alkyl, (C₂-C₄)alkenyl, (C₂-C₄)haloalkenyl, (C₁-C₄)alkoxy, (C₁-C₄)hydroxyalkyl, (C₃-C₇)cycloalkyl, or 4- to 7-membered heterocyclyl comprising 1-3 heteroatoms selected from N, O, and S, wherein the alkyl is optionally substituted with one to three R₂₇. In yet another embodiment, R_{9′} is H, (C₁-C₄)alkyl, (C₂-C₄)alkenyl, (C₂-C₄)haloalkenyl, (C₁-C₄)alkoxy, (C₁-C₄)hydroxyalkyl, or (C₃-C₇)cycloalkyl, wherein the alkyl is optionally substituted with one to three R₂₇. In another embodiment, R_{9′} is H, (C₁-C₄)alkyl, (C₂-C₄)alkenyl, (C₂-C₄)haloalkenyl, (C₁-C₄)alkoxy, (C₁-C₄)hydroxyalkyl, or (C₃-C₇)cycloalkyl, wherein the alkyl is substituted with one to three R₂₇.

In another embodiment, R_{9′} is H, (C₁-C₄)alkyl, (C₂-C₄)alkenyl, (C₂-C₄)haloalkenyl, (C₁-C₄)alkoxy, or (C₃-C₇)cycloalkyl, wherein the alkyl is optionally substituted with one to three R₂₇. In yet another embodiment, R_{9′} is H, (C₁-C₄)alkyl, (C₂-C₄)alkenyl, (C₂-C₄)haloalkenyl, or (C₃-C₆)cycloalkyl, wherein the alkyl is optionally substituted with one to three R₂₇. In another embodiment, R_{9′} is H, (C₁-C₄)alkyl, (C₂-C₄)alkenyl, (C₂-C₄)haloalkenyl, or (C₃-C₆)cycloalkyl, wherein the alkyl is optionally substituted with one to three R₂₇. In yet another embodiment, R_{9′} is H, (C₁-C₄)alkyl, (C₂-C₄)alkenyl, or (C₃-C₆)cycloalkyl, wherein the alkyl is optionally substituted with one to three R₂₇. In another embodiment, R_{9′} is H, (C₁-C₄)alkyl, or (C₃-C₆)cycloalkyl, wherein the alkyl is optionally substituted with one to three R₂₇. In yet another embodiment, R₉, is H or (C₁-C₄)alkyl optionally substituted with one to three R₂₇. In another embodiment, R_{9′} is H or (C₁-C₄)alkyl substituted with one to three R₂₇.

In another embodiment, R_{9′} is absent.

In another embodiment, X¹ is N and R_{9′} is absent. In another embodiment, when X¹ is N, R_{9′} is absent. In yet another embodiment, R_{9′} is absent when X₁ is N.

In some embodiments of the Formulae above, R₉ and R_{9′} together with the carbon atom to which they are attached form a (C₃-C₆)cycloalkyl or 4- to 6-membered heterocyclyl ring comprising 1-3 heteroatoms selected from N, O, and S. In another embodiment, R₉ and R_{9′} together with the carbon atom to which they are attached form a (C₃-C₇)cycloalkyl. In another embodiment, R₉ and R_{9′} together with the carbon atom to which they are attached form a (C₃-C₆)cycloalkyl. In yet another embodiment, R₉ and R_{9′} together with the carbon atom to which they are attached form a (C₃-C₆)cycloalkyl.

In another embodiment, R₉ and R_{9′} together with the carbon atom to which they are attached form a 4- to 7-membered heterocyclyl ring comprising 1-3 heteroatoms selected from N, O, and S. In another embodiment, R₉ and R_{9′} together with the carbon atom to which they are attached form a 4- to 6-membered heterocyclyl ring comprising 1-3 heteroatoms selected from N, O, and S. In another embodiment, R₉ and R_{9′} together with the carbon atom to which they are attached form a 4- or 5-membered heterocyclyl ring comprising 1-3 heteroatoms selected from N, O, and S. In another embodiment, R₉ and R_{9′} together with the carbon atom to which they are attached form a 5- to 7-membered heterocyclyl ring comprising 1-3 heteroatoms selected from N, O, and S.

In some embodiments of the Formulae above, R₁₀ is (C₆-C₁₀)aryl, 5- or 6-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the cycloalkyl, heterocyclyl, aryl and heteroaryl are substituted with —OR₁₃ or —NR₂₃R₁₃, and optionally substituted with one to three R₁₄. In another embodiment, R₁₀ is (C₆-C₁₀)aryl, 5- or 6-membered heteroaryl comprising 1-3 heteroatoms selected from N, O, and S, or 4- to 7-membered heterocyclyl comprising 1-3 heteroatoms selected from N, O, and S, wherein the heterocyclyl, aryl and heteroaryl are substituted with —OR₁₃ or —NR₂₃R₁₃, and optionally substituted with one to three R₁₄. In yet another embodiment, R₁₀ is (C₆-C₁₀)aryl, 5- or 6-membered heteroaryl comprising 1-3 heteroatoms selected from N, O, and S, or 4- to 7-membered heterocyclyl comprising 1-3 heteroatoms selected from N, O, and S, wherein the heterocyclyl, aryl and heteroaryl are substituted with —OR₁₃, and optionally substituted with one to three R₁₄.

In another embodiment, R₁₀ is (C₆-C₁₀)aryl or 5- or 6-membered heteroaryl comprising 1-3 heteroatoms selected from N, O, and S, wherein the aryl and heteroaryl are substituted with —OR₁₃ or —NR₂₃R₁₃, and optionally substituted with one to three R₁₄. In yet another embodiment, R₁₀ is (C₆-C₁₀)aryl or 5- or 6-membered heteroaryl comprising 1-3 heteroatoms selected from N, O, and S, wherein the aryl and heteroaryl are substituted with —OR₁₃, and optionally substituted with one to three R₁₄. In another embodiment, R₁₀ is phenyl, 5- or 6-membered heteroaryl comprising 1-3 heteroatoms selected from N, O, and S, or 4- to 7-membered heterocyclyl comprising 1-3 heteroatoms selected from N, O, and S, wherein the heterocyclyl, phenyl and heteroaryl are substituted with —OR₁₃ or —NR₂₃R₁₃, and optionally substituted with one to three R₁₄. In yet another embodiment, R₁₀ is phenyl, 5- or 6-membered heteroaryl comprising 1-3 heteroatoms selected from N, O, and S, or 4- to 7-membered heterocyclyl comprising 1-3 heteroatoms selected from N, O, and S, wherein the heterocyclyl, phenyl and heteroaryl are substituted with —OR₁₃, and optionally substituted with one to three R₁₄.

In another embodiment, R₁₀ is phenyl or 5- or 6-membered heteroaryl comprising 1-3 heteroatoms selected from N, O, and S, wherein the phenyl and heteroaryl are substituted with —OR₁₃ or —NR₂₃R₁₃, and optionally substituted with one to three R₁₄. In yet another embodiment, R₁₀ is phenyl or 5- or 6-membered heteroaryl comprising 1-3 heteroatoms selected from N, O, and S, wherein the phenyl and heteroaryl are substituted with —OR₁₃, and optionally substituted with one to three R₁₄. In another embodiment, R₁₀ is phenyl or 5-membered heteroaryl comprising 1-3 heteroatoms selected from N, O, and S, wherein the phenyl and heteroaryl are substituted with —OR₁₃ or —NR₂₃R₁₃, and optionally substituted with one to three R₁₄. In yet another embodiment, R₁₀ is phenyl or 5-membered heteroaryl comprising 1-3 heteroatoms selected from N, O, and S, wherein the phenyl and heteroaryl are substituted with —OR₁₃, and optionally substituted with one to three R₁₄.

In another embodiment, R₁₀ is phenyl or 6-membered heteroaryl comprising 1-3 heteroatoms selected from N, O, and S, wherein the phenyl and heteroaryl are substituted with —OR₁₃ or —NR₂₃R₁₃, and optionally substituted with one to three R₁₄. In yet another embodiment, R₁₀ is phenyl or 6-membered heteroaryl comprising 1-3 heteroatoms selected from N, O, and S, wherein the phenyl and heteroaryl are substituted with —OR₁₃, and optionally substituted with one to three R₁₄. In another embodiment, R₁₀ is phenyl or pyridine, wherein the phenyl and pyridine are substituted with —OR₁₃ or —NR₂₃R₁₃, and optionally substituted with one to three R₁₄. In yet another embodiment, R₁₀ is phenyl or pyridine, wherein the phenyl and pyridine are substituted with —OR₁₃, and optionally substituted with one to three R₁₄. In another embodiment, R₁₀ is phenyl substituted with —OR₁₃ or —NR₂₃R₁₃, and optionally substituted with one to three R₁₄. In yet another embodiment, R₁₀ is phenyl substituted with —OR₁₃, and optionally substituted with one to three R₁₄.

In some embodiments of the Formulae above, R₁₁ is (C₁-C₆)alkyl, (C₁-C₆)haloalkyl, or (C₁-C₆)hydroxyalkyl, wherein the alkyl is optionally substituted with one to three R₁₅. In another embodiment, R₁₁ is (C₁-C₆)alkyl, (C₁-C₆)haloalkyl, or (C₁-C₆)hydroxyalkyl, wherein the alkyl is optionally substituted with one to three R₁₅. In yet another embodiment, R₁₁ is (C₁-C₄)alkyl, (C₁-C₄)haloalkyl, or (C₁-C₄)hydroxyalkyl, wherein the alkyl is optionally substituted with one to three R₁₅. In another embodiment, R₁₁ is (C₁-C₆)alkyl, (C₁-C₆)haloalkyl, or (C₁-C₆)hydroxyalkyl, wherein the alkyl is optionally substituted with one to two R₁₅. In yet another embodiment, R₁₁ is (C₁-C₄)alkyl, (C₁-C₄)haloalkyl, or (C₁-C₄)hydroxyalkyl, wherein the alkyl is optionally substituted with one to two R₁₅. In another embodiment, R₁₁ is (C₁-C₆)alkyl or (C₁-C₆)haloalkyl, wherein the alkyl is optionally substituted with one to three R₁₅. In yet another embodiment, R₁₁ is (C₁-C₄)alkyl or (C₁-C₄)haloalkyl, wherein the alkyl is optionally substituted with one to three R₁₅. In another embodiment, R₁₁ is (C₁-C₆)alkyl or (C₁-C₆)hydroxyalkyl, wherein the alkyl is optionally substituted with one to three R₁₅. In yet another embodiment, R₁₁ is (C₁-C₄)alkyl or (C₁-C₄)hydroxyalkyl, wherein the alkyl is optionally substituted with one to three R₁₅. In another embodiment, R₁₁ is (C₁-C₆)alkyl optionally substituted with one to three R₁₅. In yet another embodiment, R₁₁ is (C₁-C₄)alkyl optionally substituted with one to three R₁₅.

In some embodiments of the Formulae above, R₁₂ is halogen, (C₁-C₃)alkyl, (C₁-C₆)alkoxy, (C₁-C₃)haloalkyl, (C₁-C₆)haloalkoxy, —OH, or CN. In another embodiment, R₁₂ is halogen, (C₁-C₃)alkyl, (C₁-C₆)alkoxy, (C₁-C₃)haloalkyl, or (C₁-C₆)haloalkoxy. In yet another embodiment, R₁₂ is halogen, —OH, or CN. In another embodiment, R₁₂ is halogen, (C₁-C₆)alkoxy, or (C₁-C₆)haloalkoxy, —OH, or CN. In yet another embodiment, R₁₂ is halogen, (C₁-C₃)alkyl, (C₁-C₃)haloalkyl, —OH, or CN. In another embodiment, R₁₂ is halogen, (C₁-C₃)alkyl, or (C₁-C₃)haloalkyl. In yet another embodiment, R₁₂ is halogen or (C₁-C₃)haloalkyl. In another embodiment, R₁₂ is halogen. In yet another embodiment, R₁₂ is Cl or F. In another embodiment, R₁₂ is halogen. In yet another embodiment, R₁₂ is Cl.

In some embodiments of the Formulae above, R₁₃ is (C₆-C₁₀)aryl, or 5- or 6-membered heteroaryl comprising 1-3 heteroatoms selected from N, O, and S, wherein the aryl and heteroaryl are substituted with R₁₆ and optionally substituted with one to three R_{16′}. In another embodiment, R₁₃ is phenyl or 5- or 6-membered heteroaryl comprising 1-3 heteroatoms selected from N, O, and S, wherein the phenyl and heteroaryl are substituted with R₁₆ and optionally substituted with one to three R_{16′}. In yet another embodiment, R₁₃ is phenyl or 5-membered heteroaryl comprising 1-3 heteroatoms selected from N, O, and S, wherein the phenyl and heteroaryl are substituted with R₁₆ and optionally substituted with one to three R_{16′}. In another embodiment, R₁₃ is phenyl or 6-membered heteroaryl comprising 1-3 heteroatoms selected from N, O, and S, wherein the phenyl and heteroaryl are substituted with R₁₆ and optionally substituted with one to three R_{16′}.

In another embodiment, R₁₃ is phenyl or 6-membered heteroaryl comprising 1-2 N atoms, wherein the phenyl and heteroaryl are substituted with R₁₆ and optionally substituted with one to three R_{16′}. In yet another embodiment, R₁₃ is phenyl or pyridine, wherein the phenyl and pyridine are substituted with R₁₆ and optionally substituted with one to three R_{16′}. In another embodiment, R₁₃ is phenyl or pyridine, wherein the phenyl and pyridine are substituted with R₁₆ and optionally substituted with one to two R_{16′}. In yet another embodiment, R₁₃ is phenyl or pyridine, wherein the phenyl and pyridine are substituted with R₁₆. In another embodiment, R₁₃ is phenyl is substituted with R₁₆ and optionally substituted with one to three R_{16′}. In yet another embodiment, R₁₃ is phenyl is substituted with R₁₆ and optionally substituted with one to two R_{16′}. In another embodiment, R₁₃ is phenyl is substituted with R₁₆.

In some embodiments of the Formulae above, each R₁₄ is independently at each occurrence halogen, (C₁-C₄)alkyl, (C₁-C₄)alkoxy, (C₁-C₄)haloalkyl, (C₁-C₄)haloalkoxy, oxo, —OH, or CN. In another embodiment, each R₁₄ is independently at each occurrence halogen, (C₁-C₄)alkyl, (C₁-C₄)alkoxy, (C₁-C₄)haloalkyl, (C₁-C₄)haloalkoxy, —OH, or CN. In yet another embodiment, each R₁₄ is independently at each occurrence halogen, (C₁-C₄)alkyl, (C₁-C₄)alkoxy, (C₁-C₄)haloalkyl, (C₁-C₄)haloalkoxy, oxo, or —OH. In another embodiment, each R₁₄ is independently at each occurrence halogen, (C₁-C₄)alkyl, (C₁-C₄)alkoxy, (C₁-C₄)haloalkyl, (C₁-C₄)haloalkoxy, oxo, or CN. In yet another embodiment, each R₁₄ is independently at each occurrence halogen, (C₁-C₄)alkyl, (C₁-C₄)alkoxy, (C₁-C₄)haloalkyl, (C₁-C₄)haloalkoxy, or oxo. In another embodiment, each R₁₄ is independently at each occurrence halogen, (C₁-C₄)alkyl, (C₁-C₄)alkoxy, (C₁-C₄)haloalkyl, or (C₁-C₄)haloalkoxy. In yet another embodiment, each R₁₄ is independently at each occurrence halogen, (C₁-C₄)alkyl, (C₁-C₄)haloalkyl, or (C₁-C₄)haloalkoxy. In another embodiment, each R₁₄ is independently at each occurrence halogen, (C₁-C₄)alkyl, (C₁-C₄)alkoxy, or (C₁-C₄)haloalkyl.

In yet another embodiment, each R₁₄ is independently at each occurrence halogen, (C₁-C₄)alkyl, or (C₁-C₄)haloalkyl. In another embodiment, each R₁₄ is independently at each occurrence halogen, (C₁-C₄)alkyl, or (C₁-C₄)haloalkoxy. In yet another embodiment, each R₁₄ is independently at each occurrence halogen, or (C₁-C₄)alkyl. In another embodiment, each R₁₄ is independently at each occurrence F, Cl, (C₁-C₄)alkyl, (C₁-C₄)alkoxy, (C₁-C₄)haloalkyl, or (C₁-C₄)haloalkoxy. In yet another embodiment, each R₁₄ is independently at each occurrence F, Cl, (C₁-C₄)alkyl, (C₁-C₄)haloalkyl, or (C₁-C₄)haloalkoxy. In another embodiment, each R₁₄ is independently at each occurrence F, Cl, (C₁-C₄)alkyl, or (C₁-C₄)haloalkoxy. In yet another embodiment, each R₁₄ is independently at each occurrence F, Cl, or (C₁-C₄)alkyl. In another embodiment, each R₁₄ is independently at each occurrence F, Cl, or (C₁-C₃)alkyl. In yet another embodiment, each R₁₄ is independently at each occurrence is halogen.

In another embodiment, when R₁₀ is cycloalkyl or heterocyclyl, two R₁₄ together, when attached to the same carbon atom, together form ═(O).

In some embodiments of the Formulae above, each R₁₅ is independently at each occurrence (C₁-C₃)alkoxy, (C₁-C₃)haloalkoxy, —C(O)R₁₉, —S(O)_{q}(C₁-C₃)alkyl, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, —NR₁₇C(O)OR₁₈, (C₃-C₇)cycloalkyl, or 4- to 7-membered heterocyclyl comprising 1-3 heteroatoms selected from N, O, and S, wherein the cycloalkyl and heterocyclyl are optionally substituted with one to four R₂₁. In another embodiment, each R₁₅ is independently at each occurrence (C₁-C₃)alkoxy, (C₁-C₃)haloalkoxy, —C(O)R₁₉, —S(O)_{q}(C₁-C₃)alkyl, —C(O)OH, —C(O)O(C₁-C₆)alkyl, or —OC(O)(C₁-C₆)alkyl. In yet another embodiment, each R₁₅ is independently at each occurrence —NR₁₇R₁₈, —C(O)NR₁₇R₁₈, —NR₁₇C(O)R₂₀, —NR₁₇C(O)OR₁₈, (C₃-C₇)cycloalkyl, or 4- to 7-membered heterocyclyl comprising 1-3 heteroatoms selected from N, O, and S, wherein the cycloalkyl and heterocyclyl are optionally substituted with one to four R₂₁.

In another embodiment, each R₁₅ is independently at each occurrence (C₁-C₃)alkoxy, (C₁-C₃)haloalkoxy, —C(O)R₁₉, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, —NR₁₇C(O)OR₁₈, (C₃-C₇)cycloalkyl, or 4- to 7-membered heterocyclyl comprising 1-3 heteroatoms selected from N, O, and S, wherein the cycloalkyl and heterocyclyl are optionally substituted with one to four R₂₁. In yet another embodiment, each R₁₅ is independently at each occurrence (C₁-C₃)alkoxy, (C₁-C₃)haloalkoxy, —C(O)R₁₉, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, —NR₁₇C(O)OR₁₈, or 4- to 7-membered heterocyclyl comprising 1-3 heteroatoms selected from N, O, and S, wherein the heterocyclyl is optionally substituted with one to four R₂₁.

In another embodiment, each R₁₅ is independently at each occurrence (C₁-C₃)alkoxy, (C₁-C₃)haloalkoxy, —C(O)R₁₉, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, or 4- to 7-membered heterocyclyl comprising 1-3 heteroatoms selected from N, O, and S, wherein the heterocyclyl is optionally substituted with one to four R₂₁. In yet another embodiment, each R₁₅ is independently at each occurrence (C₁-C₃)alkoxy, (C₁-C₃)haloalkoxy, —C(O)R₁₉, —C(O)OH, —C(O)O(C₁-C₆)alkyl, —OC(O)(C₁-C₆)alkyl, —NR₁₇R₁₈, —C(O)NR₁₇R₁₈, or 4- to 7-membered heterocyclyl comprising 1-3 heteroatoms selected from N, O, and S, wherein the heterocyclyl is optionally substituted with one to four R₂₁.

In some embodiments of the Formulae above, R₁₆ is —C(O)NR₃₁R₃₂, (C₆-C₁₀)aryl, or 5- to 7-membered heteroaryl comprising 1-3 heteroatoms selected from N, O, and S, wherein the aryl and heteroaryl are optionally substituted with one to three R₂₆. In another embodiment, R₁₆ is —C(O)NR₃₁R₃₂, phenyl, or 5- to 7-membered heteroaryl comprising 1-3 heteroatoms selected from N, O, and S, wherein the phenyl and heteroaryl are optionally substituted with one to three R₂₆. In yet another embodiment, R₁₆ is —C(O)NR₃₁R₃₂, phenyl, or 5- to 6-membered heteroaryl comprising 1-3 heteroatoms selected from N, O, and S, wherein the phenyl and heteroaryl are optionally substituted with one to three R₂₆. In another embodiment, R₁₆ is phenyl or 5- to 6-membered heteroaryl comprising 1-3 heteroatoms selected from N, O, and S, wherein the phenyl and heteroaryl are optionally substituted with one to three R₂₆.

In another embodiment, R₁₆ is —C(O)NR₃₁R₃₂ or 5- to 6-membered heteroaryl comprising 1-3 heteroatoms selected from N, O, and S, optionally substituted with one to three R₂₆. In yet another embodiment, R₁₆ is —C(O)NR₃₁R₃₂. In another embodiment, R₁₆ is 5- to 6-membered heteroaryl comprising 1-3 heteroatoms selected from N, O, and S, optionally substituted with one to four R₂₆. In yet another embodiment, R₁₆ is 5- to 6-membered heteroaryl comprising 1-3 heteroatoms selected from N and O, optionally substituted with one to four R₂₆. In another embodiment, R₁₆ is 5- to 6-membered heteroaryl comprising 1-3 N atoms, optionally substituted with one to four R₂₆. In yet another embodiment, R₁₆ is imidazole, triazole, pyridine, pyrimidine or pyrazine, wherein each is optionally substituted with one to four R₂₆.

In some embodiments of the Formulae above, each R_{16′} is independently at each occurrence halogen, (C₁-C₆)alkyl, (C₁-C₆)alkoxy, (C₁-C₆)haloalkyl, (C₁-C₆)haloalkoxy, or —OH. In another embodiment, each R_{16′} is independently at each occurrence halogen, (C₁-C₆)alkyl, (C₁-C₆)alkoxy, (C₁-C₆)haloalkyl, (C₁-C₆)haloalkoxy, or —OH. In yet another embodiment, each R_{16′} is independently at each occurrence halogen, (C₁-C₃)alkyl, (C₁-C₃)alkoxy, (C₁-C₃)haloalkyl, (C₁-C₃)haloalkoxy, —OH or CN. In another embodiment, each R_{16′} is independently at each occurrence halogen, (C₁-C₆)alkyl, (C₁-C₆)alkoxy, (C₁-C₆)haloalkyl, (C₁-C₆)haloalkoxy, or —OH

In yet another embodiment, each R_{16′} is independently at each occurrence halogen, (C₁-C₆)alkyl, (C₁-C₆)alkoxy, (C₁-C₆)haloalkyl, or (C₁-C₆)haloalkoxy. In another embodiment, each R_{16′} is independently at each occurrence halogen, (C₁-C₆)alkyl, (C₁-C₆)haloalkyl, (C₁-C₆)haloalkoxy, or —OH. In yet another embodiment, each R_{16′} is independently at each occurrence halogen, (C₁-C₆)alkyl, (C₁-C₆)alkoxy, (C₁-C₆)haloalkyl, or —OH. In another embodiment, each R_{16′} is independently at each occurrence halogen, (C₁-C₆)alkyl, or (C₁-C₆)haloalkyl. In yet another embodiment, each R_{16′} is independently at each occurrence halogen or (C₁-C₆)alkyl. In another embodiment, each R_{16′} is independently at each occurrence halogen or (C₁-C₃)alkyl.

In some embodiments of the Formulae above, R₁₆ and R_{16′} together with the atoms to which they are attached form a 5- to 7-membered heterocyclyl ring optionally substituted with one to three substituents independently selected from ═(O) and R₃₄. In another embodiment, R₁₆ and R_{16′} together with the atoms to which they are attached form a 5- or 6-membered heterocyclyl ring optionally substituted with one to three substituents independently selected from ═(O) and R₃₄. In another embodiment, R₁₆ and R_{16′} together with the atoms to which they are attached form a 6- or 7-membered heterocyclyl ring optionally substituted with one to three substituents independently selected from ═(O) and R₃₄.

In some embodiments of the Formulae above, R₁₇ is H or (C₁-C₆)alkyl optionally substituted with one to two substituents each independently selected from (C₁-C₆)alkoxy and —C(O)O(C₁-C₆)alkyl. In another embodiment, R₁₇ is H or (C₁-C₄)alkyl optionally substituted with one to two substituents each independently selected from (C₁-C₄)alkoxy and —C(O)O(C₁-C₄)alkyl. In another embodiment, R₁₇ is H or (C₁-C₃)alkyl optionally substituted with one to two substituents each independently selected from (C₁-C₃)alkoxy and —C(O)O(C₁-C₃)alkyl.

In some embodiments of the Formulae above, R₁₈ is H or (C₁-C₆)alkyl optionally substituted with one to two substituents each independently selected from (C₁-C₆)alkoxy and —C(O)O(C₁-C₆)alkyl. In another embodiment, R₁₈ is H or (C₁-C₄)alkyl optionally substituted with one to two substituents each independently selected from (C₁-C₄)alkoxy and —C(O)O(C₁-C₄)alkyl. In another embodiment, R₁₈ is H or (C₁-C₃)alkyl optionally substituted with one to two substituents each independently selected from (C₁-C₃)alkoxy and —C(O)O(C₁-C₃)alkyl.

In some embodiments of the Formulae above, R₁₉ is (C₃-C₇)cycloalkyl or 4- to 7-membered heterocyclyl comprising 1-3 heteroatoms selected from N, O, and S, optionally substituted with one to four R₂₂. In another embodiment, R₁₉ is (C₃-C₇)cycloalkyl or 4- to 7-membered heterocyclyl comprising 1-3 heteroatoms selected from N, O, and S, optionally substituted with one to three R₂₂. In yet another embodiment, R₁₉ is (C₃-C₆)cycloalkyl or 4- to 7-membered heterocyclyl comprising 1-3 heteroatoms selected from N, O, and S, optionally substituted with one to three R₂₂. In another embodiment, R₁₉ is (C₃-C₇)cycloalkyl or 4- to 6-membered heterocyclyl comprising 1-3 heteroatoms selected from N, O, and S, optionally substituted with one to three R₂₂. In ye another embodiment, R₁₉ is (C₃-C₆)cycloalkyl or 4- to 6-membered heterocyclyl comprising 1-3 heteroatoms selected from N, O, and S, optionally substituted with one to three R₂₂.

In another embodiment, R₁₉ is (C₃-C₇)cycloalkyl optionally substituted with one to three R₂₂. In yet another embodiment, R₁₉ is (C₃-C₆)cycloalkyl optionally substituted with one to three R₂₂. In another embodiment, R₁₉ is (C₄-C₇)cycloalkyl optionally substituted with one to three R₂₂. In yet another embodiment, R₁₉ is (C₄-C₆)cycloalkyl optionally substituted with one to three R₂₂. In another embodiment, R₁₉ is 4- to 7-membered heterocyclyl comprising 1-3 heteroatoms selected from N, O, and S, optionally substituted with one to three R₂₂. In yet another embodiment, R₁₉ is 5- to 7-membered heterocyclyl comprising 1-3 heteroatoms selected from N, O, and S, optionally substituted with one to three R₂₂. In another embodiment, R₁₉ is 4- to 6-membered heterocyclyl comprising 1-3 heteroatoms selected from N, O, and S, optionally substituted with one to three R₂₂.

In some embodiments of the Formulae above, R₂₀ is —(CH₂CH₂O)ₘCH₂CH₂ONH₂, —(CH₂CH₂O)ₘCH₂CH₂ONH(C₁-C₆)alkyl, or (C₁-C₆)alkyl optionally substituted with one to three —NR₂₃C(O)R₂₄. In another embodiment, R₂₀ is —(CH₂CH₂O)ₘCH₂CH₂ONH₂, or —(CH₂CH₂O)ₘCH₂CH₂ONH(C₁-C₆)alkyl. In yet another embodiment, R₂₀ is —(CH₂CH₂O)ₘCH₂CH₂ONH(C₁-C₆)alkyl or (C₁-C₆)alkyl optionally substituted with one to three —NR₂₃C(O)R₂₄. In another embodiment, R₂₀ is —(CH₂CH₂O)ₘCH₂CH₂ONH₂ or (C₁-C₆)alkyl optionally substituted with one to three —NR₂₃C(O)R₂₄. In another embodiment, R₂₀ is (C₁-C₆)alkyl optionally substituted with one to three —NR₂₃C(O)R₂₄.

In some embodiments of the Formulae above, each R₂₁ is independently at each occurrence (C₁-C₄)alkyl, (C₁-C₄)alkoxy, (C₁-C₄)haloalkyl, (C₁-C₄)haloalkoxy, halogen, ═(O), or —OH. In another embodiment, each R₂₁ is independently at each occurrence (C₁-C₄)alkyl, (C₁-C₄)alkoxy, (C₁-C₄)haloalkyl, (C₁-C₄)haloalkoxy, halogen, or ═(O). In yet another embodiment, each R₂₁ is independently at each occurrence (C₁-C₄)alkyl, (C₁-C₄)alkoxy, (C₁-C₄)haloalkyl, or (C₁-C₄)haloalkoxy. In another embodiment, each R₂₁ is independently at each occurrence, halogen, ═(O), or —OH. In yet another embodiment, each R₂₁ is independently at each occurrence (C₁-C₄)alkyl, (C₁-C₄)haloalkyl, halogen, ═(O), or —OH. In another embodiment, each R₂₁ is independently at each occurrence (C₁-C₄)alkyl, halogen, ═(O), or —OH. In yet another embodiment, each R₂₁ is independently at each occurrence (C₁-C₄)alkyl, halogen, or ═(O). In another embodiment, each R₂₁ is independently at each occurrence halogen or ═(O). In yet another embodiment, each R₂₁ is independently at each occurrence (C₁-C₄)alkyl, halogen, or —OH.

In some embodiments of the Formulae above, each R₂₂ is independently at each occurrence (C₁-C₄)alkyl, (C₁-C₄)haloalkyl, halogen, or —OH. In another embodiment, each R₂₂ is independently at each occurrence (C₁-C₄)alkyl, (C₁-C₄)haloalkyl, or halogen. In yet another embodiment, each R₂₂ is independently at each occurrence (C₁-C₄)alkyl, halogen, or —OH. In another embodiment, each R₂₂ is independently at each occurrence (C₁-C₄)alkyl or (C₁-C₄)haloalkyl. In yet another embodiment, each R₂₂ is independently at each occurrence, halogen or —OH. In another embodiment, each R₂₂ is independently at each occurrence (C₁-C₄)alkyl or halogen.

In some embodiments of the Formulae above, two R₂₂ together when on the same atom form a (C₃-C₆)spirocycloalkyl or 4- to 7-membered spiroheterocyclyl comprising 1-3 heteroatoms selected from N, O, and S. In another embodiment, two R₂₂ together when on the same atom form a (C₃-C₇)spirocycloalkyl or 4- to 6-membered spiroheterocyclyl comprising 1-3 heteroatoms selected from N, O, and S. In another embodiment, two R₂₂ together when on the same atom form a (C₃-C₆)spirocycloalkyl or 4- to 6-membered spiroheterocyclyl comprising 1-3 heteroatoms selected from N, O, and S. In another embodiment, two R₂₂ together when on the same atom form a (C₃-C₇)spirocycloalkyl. In another embodiment, two R₂₂ together when on the same atom form a (C₃-C₆)spirocycloalkyl. In another embodiment, two R₂₂ together when on the same atom form a (C₃-C₅)spirocycloalkyl. In another embodiment, two R₂₂ together when on the same atom form a (C₃-C₄)spirocycloalkyl. In another embodiment, two R₂₂ together when on the same atom form a (C₄-C₇)spirocycloalkyl. In another embodiment, two R₂₂ together when on the same atom form a (C₅-C₇)spirocycloalkyl. In another embodiment, two R₂₂ together when on the same atom form a (C₆-C₇)spirocycloalkyl.

In another embodiment, two R₂₂ together when on the same atom form a 4- to 7-membered spiroheterocyclyl comprising 1-3 heteroatoms selected from N, O, and S. In yet another embodiment, two R₂₂ together when on the same atom form a 4- to 6-membered spiroheterocyclyl comprising 1-3 heteroatoms selected from N, O, and S. In another embodiment, two R₂₂ together when on the same atom form a 4- or 5-membered spiroheterocyclyl comprising 1-3 heteroatoms selected from N, O, and S. In yet another embodiment, two R₂₂ together when on the same atom form a 5- to 7-membered spiroheterocyclyl comprising 1-3 heteroatoms selected from N, O, and S. In another embodiment, two R₂₂ together when on the same atom form a 6- or 7-membered spiroheterocyclyl comprising 1-3 heteroatoms selected from N, O, and S. In yet another embodiment, two R₂₂ together when on the same atom form a 5- or 6-membered spiroheterocyclyl comprising 1-3 heteroatoms selected from N, O, and S.

In some embodiments of the Formulae above, R₂₃ is H or (C₁-C₃)alkyl. In another embodiment, R₂₃ is (C₁-C₃)alkyl. In another embodiment, R₂₃ is H, methyl, ethyl, n-propyl, or i-propyl. In another embodiment, R₂₃ is H, methyl, or ethyl. In another embodiment, R₂₃ is H or methyl. In another embodiment, R₂₃ is H.

In some embodiments of the Formulae above, R₂₄ is H or (C₁-C₆)alkyl optionally substituted with one to three R₂₅. In another embodiment, R₂₄ is H. In yet another embodiment, R₂₄ is (C₁-C₆)alkyl optionally substituted with one to three R₂₅. In another embodiment, R₂₄ is H or (C₁-C₆)alkyl optionally substituted with one to two R₂₅. In yet another embodiment, R₂₄ is (C₁-C₆)alkyl optionally substituted with one to two R₂₅.

In some embodiments of the Formulae above, each R₂₅ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with one to three substituents independently selected from (C₁-C₆)alkyl, (C₁-C₆)haloalkyl, and ═(O). In another embodiment, each R₂₅ is independently at each occurrence (C₃-C₇)cycloalkyl or 8- to 10-membered bicyclic heterocyclyl comprising 1-4 heteroatoms selected from N, O, and S, wherein the cycloalkyl and heterocyclyl are optionally substituted with one to three substituents independently selected from (C₁-C₆)alkyl, (C₁-C₆)haloalkyl, and ═(O).

In another embodiment, each R₂₅ is independently at each occurrence (C₃-C₇)cycloalkyl optionally substituted with one to three substituents independently selected from (C₁-C₆)alkyl, (C₁-C₆)haloalkyl, and ═(O). In yet another embodiment, each R₂₅ is independently at each occurrence (C₄-C₇)cycloalkyl optionally substituted with one to three substituents independently selected from (C₁-C₆)alkyl, (C₁-C₆)haloalkyl, and ═(O). In another embodiment, each R₂₅ is independently at each occurrence (C₃-C₆)cycloalkyl optionally substituted with one to three substituents independently selected from (C₁-C₆)alkyl, (C₁-C₆)haloalkyl, and ═(O). In yet another embodiment, each R₂₅ is independently at each occurrence (C₆-C₇)cycloalkyl optionally substituted with one to three substituents independently selected from (C₁-C₆)alkyl, (C₁-C₆)haloalkyl, and ═(O). In another embodiment, each R₂₅ is independently at each occurrence (C₄-C₆)cycloalkyl optionally substituted with one to three substituents independently selected from (C₁-C₆)alkyl, (C₁-C₆)haloalkyl, and ═(O).

In another embodiment, each R₂₅ is independently at each occurrence 4- to 10-membered mono or bicyclic heterocyclyl comprising 1-4 heteroatoms selected from N, O, and S, wherein the cycloalkyl and heterocyclyl are optionally substituted with one to three substituents independently selected from (C₁-C₆)alkyl, (C₁-C₆)haloalkyl, and ═(O). In yet another embodiment, each R₂₅ is independently at each occurrence 4- to 7-membered mono heterocyclyl comprising 1-3 heteroatoms selected from N, O, and S, wherein the cycloalkyl and heterocyclyl are optionally substituted with one to three substituents independently selected from (C₁-C₆)alkyl, (C₁-C₆)haloalkyl, and ═(O). In another embodiment, each R₂₅ is independently at each occurrence 8- to 10-membered bicyclic heterocyclyl comprising 1-4 heteroatoms selected from N, O, and S, optionally substituted with one to three substituents independently selected from (C₁-C₆)alkyl, (C₁-C₆)haloalkyl, and ═(O).

In some embodiments of the Formulae above, each R₂₆ is independently at each occurrence (C₁-C₆)alkyl optionally substituted with one to three R₂₉, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one to three substituents each independently selected from (C₁-C₆)alkyl, (C₁-C₆)haloalkyl, —NH₂, —N(H)(C₁-C₆)alkyl, —N((C₁-C₆)alkyl)₂, —N(H)(C₁-C₆)haloalkyl, —N((C₁-C₆)haloalkyl)₂, halogen, and —OH.

In another embodiment, each R₂₆ is independently at each occurrence (C₁-C₆)alkyl optionally substituted with one to three R₂₉, (C₂-C₆)alkenyl, (C₂-C₆)alkynyl, (C₁-C₆)alkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, or 4- to 7-membered heterocyclyl comprising 1-3 heteroatoms selected from N, O, and S, wherein the cycloalkyl and heterocyclyl are optionally substituted with one to three substituents each independently selected from (C₁-C₆)alkyl, (C₁-C₆)haloalkyl, —NH₂, —N(H)(C₁-C₆)alkyl, —N((C₁-C₆)alkyl)₂, —N(H)(C₁-C₆)haloalkyl, —N((C₁-C₆)haloalkyl)₂, halogen, and —OH.

In yet another embodiment, each R₂₆ is independently at each occurrence (C₁-C₆)alkyl optionally substituted with one to three R₂₉, (C₂-C₆)alkenyl, (C₂-C₆)alkynyl, (C₁-C₆)alkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one to three substituents each independently selected from (C₁-C₆)alkyl, (C₁-C₆)haloalkyl, —NH₂, —N(H)(C₁-C₆)alkyl, —N((C₁-C₆)alkyl)₂, —N(H)(C₁-C₆)haloalkyl, —N((C₁-C₆)haloalkyl)₂, halogen, and —OH.

In another embodiment, each R₂₆ is independently at each occurrence (C₁-C₆)alkyl optionally substituted with one to three R₂₉, (C₂-C₆)alkenyl, (C₂-C₆)alkynyl, (C₁-C₆)alkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one to three substituents each independently selected from (C₁-C₆)alkyl, (C₁-C₆)haloalkyl, —NH₂, —N(H)(C₁-C₆)haloalkyl, —N((C₁-C₆)haloalkyl)₂, halogen, and —OH.

In yet another embodiment, each R₂₆ is independently at each occurrence (C₁-C₆)alkyl optionally substituted with one to three R₂₉, (C₂-C₆)alkenyl, (C₂-C₆)alkynyl, (C₁-C₆)alkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one to three substituents each independently selected from (C₁-C₆)alkyl, (C₁-C₆)haloalkyl, —NH₂, —N(H)(C₁-C₆)haloalkyl, —N((C₁-C₆)haloalkyl)₂, and —OH.

In another embodiment, each R₂₆ is independently at each occurrence (C₁-C₆)alkyl optionally substituted with one to three R₂₉, (C₂-C₆)alkenyl, (C₂-C₆)alkynyl, (C₁-C₆)alkoxy, (C₁—C)hydroxyalkyl, —C(O)NR₃₁R₃₂, -4- to 7-membered heterocyclyl comprising 1-3 heteroatoms selected from N, O, and S, wherein the heterocyclyl is optionally substituted with one to three substituents each independently selected from (C₁-C₆)alkyl, (C₁-C₆)haloalkyl, —NH₂, —N(H)(C₁-C₆)haloalkyl, —N((C₁-C₆)haloalkyl)₂, and —OH. In yet another embodiment, each R₂₉ is independently at each occurrence (C₁-C₆)alkyl optionally substituted with one to three R₂₉, (C₂-C₆)alkenyl, (C₂-C₆)alkynyl, (C₁-C₆)alkoxy, (C₁-C₆)hydroxyalkyl, 4- to 7-membered heterocyclyl comprising 1-3 heteroatoms selected from N, O, and S, wherein the heterocyclyl is optionally substituted with one to three substituents each independently selected from (C₁-C₆)alkyl, (C₁-C₆)haloalkyl, —NH₂, —N(H)(C₁-C₆)haloalkyl, —N((C₁-C₆)haloalkyl)₂, and —OH.

In another embodiment, each R₂₆ is independently at each occurrence (C₁-C₆)alkyl optionally substituted with one to three R₂₉, (C₂-C₆)alkenyl, (C₂-C₆)alkynyl, (C₁-C₆)alkoxy, (C₁-C₆)hydroxyalkyl, 4- to 7-membered heterocyclyl comprising 1-3 heteroatoms selected from N, O, and S, wherein the heterocyclyl is optionally substituted with one to three substituents each independently selected from (C₁-C₆)alkyl, (C₁-C₆)haloalkyl, and —OH. In another embodiment, each R₂₆ is independently at each occurrence (C₁-C₆)alkyl optionally substituted with one to three R₂₉.

In some embodiments of the Formulae above, two R₂₆, when on adjacent atoms, together with the atoms to which they are attached form a (C₃-C₇)carbocyclyl or 4- to 7-membered heterocyclyl ring comprising 1-3 heteroatoms selected from N, O, and S, optionally substituted with one to three R₃₃. In another embodiment, two R₂₆, when on adjacent atoms, together with the atoms to which they are attached form a (C₃-C₆)carbocyclyl or 4- to 6-membered heterocyclyl ring comprising 1-3 heteroatoms selected from N, O, and S, optionally substituted with one to three R₃₃. In yet another embodiment, two R₂₆, when on adjacent atoms, together with the atoms to which they are attached form a (C₄-C₆)carbocyclyl or 4- to 6-membered heterocyclyl ring comprising 1-3 heteroatoms selected from N, O, and S, optionally substituted with one to three R₃₃. In another embodiment, two R₂₆, when on adjacent atoms, together with the atoms to which they are attached form a (C₅-C₆)carbocyclyl or 5- or 6-membered heterocyclyl ring comprising 1-3 heteroatoms selected from N, O, and S, optionally substituted with one to three R₃₃.

In another embodiment, two R₂₆, when on adjacent atoms, together with the atoms to which they are attached form a (C₃-C₇)carbocyclyl optionally substituted with one to three R₃₃. In yet another embodiment, two R₂₆, when on adjacent atoms, together with the atoms to which they are attached form a (C₄-C₇)carbocyclyl optionally substituted with one to three R₃₃. In another embodiment, two R₂₆, when on adjacent atoms, together with the atoms to which they are attached form a (C₃-C₆)carbocyclyl optionally substituted with one to three R₃₃. In yet another embodiment, two R₂₆, when on adjacent atoms, together with the atoms to which they are attached form a (C₄-C₆)carbocyclyl optionally substituted with one to three R₃₃. In another embodiment, two R₂₆, when on adjacent atoms, together with the atoms to which they are attached form a (C₅-C₇)carbocyclyl optionally substituted with one to three R₃₃.

In another embodiment, two R₂₆, when on adjacent atoms, together with the atoms to which they are attached form a 4- to 7-membered heterocyclyl ring comprising 1-3 heteroatoms selected from N, O, and S, optionally substituted with one to three R₃₃. In another embodiment, two R₂₆, when on adjacent atoms, together with the atoms to which they are attached form a 4- to 6-membered heterocyclyl ring comprising 1-3 heteroatoms selected from N, O, and S, optionally substituted with one to three R₃₃. In another embodiment, two R₂₆, when on adjacent atoms, together with the atoms to which they are attached form a 5- to 7-membered heterocyclyl ring comprising 1-3 heteroatoms selected from N, O, and S, optionally substituted with one to three R₃₃. In another embodiment, two R₂₆, when on adjacent atoms, together with the atoms to which they are attached form a 5- or 6-membered heterocyclyl ring comprising 1-3 heteroatoms selected from N, O, and S, optionally substituted with one to three R₃₃.

In some embodiments of the Formulae above, each R₂₇ is independently at each occurrence CN, (C₆-C₁₀)aryl, 5- to 7-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl and heterocyclyl are optionally substituted with one to four R₂₃. In another embodiment, each R₂₇ is independently at each occurrence CN, (C₆-C₁₀)aryl, 5- or 6-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl and heterocyclyl are optionally substituted with one to four R₂₃.

In another embodiment, each R₂₇ is independently at each occurrence CN, (C₆-C₁₀)aryl, 5- or 6-membered heteroaryl comprising 1-3 heteroatoms selected from N, O, and S, or (C₃-C₇)cycloalkyl, wherein the aryl, heteroaryl, and cycloalkyl are optionally substituted with one to four R₂₈. In yet another embodiment, each R₂₇ is independently at each occurrence (C₆-C₁₀)aryl, 5- or 6-membered heteroaryl comprising 1-3 heteroatoms selected from N, O, and S, or (C₃-C₇)cycloalkyl, wherein the aryl, heteroaryl, and cycloalkyl are optionally substituted with one to four R₂₈.

In another embodiment, each R₂₇ is independently at each occurrence phenyl, 5- or 6-membered heteroaryl comprising 1-3 heteroatoms selected from N, O, and S, or (C₃-C₇)cycloalkyl, wherein the phenyl, heteroaryl, and cycloalkyl are optionally substituted with one to four R₂₈. In yet another embodiment, each R₂₇ is independently at each occurrence (C₆-C₁₀)aryl or 5- or 6-membered heteroaryl comprising 1-3 heteroatoms selected from N, O, and S, wherein the aryl and heteroaryl, are optionally substituted with one to four R₂₈. In another embodiment, each R₂₇ is independently at each occurrence phenyl or 5- or 6-membered heteroaryl comprising 1-3 heteroatoms selected from N, O, and S, wherein the phenyl and heteroaryl, are optionally substituted with one to four R₂₈.

In another embodiment, each R₂₇ is independently at each occurrence (C₆-C₁₀)aryl or 5- or 6-membered heteroaryl comprising 1-3 heteroatoms selected from N and O, wherein the aryl and heteroaryl, are optionally substituted with one to four R₂₈. In yet another embodiment, each R₂₇ is independently at each occurrence phenyl or 5- or 6-membered heteroaryl comprising 1-3 heteroatoms selected from N and O, wherein the aryl and heteroaryl, are optionally substituted with one to four R₂₈. In another embodiment, each R₂₇ is independently at each occurrence (C₆-C₁₀)aryl or 5- or 6-membered heteroaryl comprising 1-3 heteroatoms N, wherein the aryl and heteroaryl, are optionally substituted with one to four R₂₈. In another embodiment, each R₂₇ is independently at each occurrence phenyl or 5- or 6-membered heteroaryl comprising 1-3 heteroatoms N, wherein the aryl and heteroaryl, are optionally substituted with one to four R₂₈.

In some embodiments of the Formulae above, each R₂₈ is independently at each occurrence (C₁-C₃)alkyl, (C₁-C₃)haloalkyl, (C₁-C₃)alkoxy, (C₁-C₃)haloalkoxy, (C₁-C₃)hydroxyalkyl, halogen, oxo, or CN. In yet another embodiment, each R₂₈ is independently at each occurrence (C₁-C₃)alkyl, (C₁-C₃)haloalkyl, (C₁-C₃)alkoxy, (C₁-C₃)haloalkoxy, halogen, oxo, or CN. In another embodiment, each R₂₈ is independently at each occurrence (C₁-C₃)alkyl, (C₁-C₃)haloalkyl, (C₁-C₃)haloalkoxy, halogen, oxo, or CN. In yet another embodiment, each R₂₈ is independently at each occurrence (C₁-C₃)alkyl, (C₁-C₃)haloalkyl, (C₁-C₃)haloalkoxy, halogen, or oxo. In another embodiment, each R₂₈ is independently at each occurrence (C₁-C₃)alkyl, (C₁-C₃)haloalkyl, halogen, or oxo. In yet another embodiment, each R₂₈ is independently at each occurrence (C₁-C₃)alkyl, (C₁-C₃)haloalkyl, (C₁-C₃)haloalkoxy, or halogen.

In some embodiments of the Formulae above, when R₂₇ is cycloalkyl, two R₂₈ together with the atoms to which they are attached form a (C₄-C₇)cycloalkyl or a 4- to 7-membered heterocyclyl ring comprising 1-3 heteroatoms selected from N, O, and S. In another embodiment, when R₂₇ is heterocyclyl, two R₂₈ together with the atoms to which they are attached form a (C₄-C₇)cycloalkyl or a 4- to 7-membered heterocyclyl ring comprising 1-3 heteroatoms selected from N, O, and S. In another embodiment, when R₂₇ is cycloalkyl or heterocyclyl, two R₂₈ together with the atoms to which they are attached form a (C₄-C₆)cycloalkyl or a 4- to 6-membered heterocyclyl ring comprising 1-3 heteroatoms selected from N, O, and S. In another embodiment, when R₂₇ is cycloalkyl or heterocyclyl, two R₂₈ together with the atoms to which they are attached form a (C₄-C₆)cycloalkyl or a 4- or 5-membered heterocyclyl ring comprising 1-3 heteroatoms selected from N, O, and S.

In another embodiment, when R₂₇ is cycloalkyl or heterocyclyl, two R₂₈ together with the atoms to which they are attached form a (C₅-C₇)cycloalkyl or a 5- to 7-membered heterocyclyl ring comprising 1-3 heteroatoms selected from N, O, and S. In another embodiment, when R₂₇ is cycloalkyl or heterocyclyl, two R₂₈ together with the atoms to which they are attached form a (C₆-C₇)cycloalkyl or a 6- or 7-membered heterocyclyl ring comprising 1-3 heteroatoms selected from N, O, and S.

In another embodiment, when R₂₇ is cycloalkyl or heterocyclyl, two R₂₈ together with the atoms to which they are attached form a (C₄-C₇)cycloalkyl or a 4- to 7-membered heterocyclyl ring comprising 1-3 heteroatoms selected from N, O, and S.

In another embodiment, when R₂₇ is cycloalkyl or heterocyclyl, two R₂₈ together, when attached to the same carbon atom, together form ═(O);

In some embodiments of the Formulae above, each R₂₉ is independently at each occurrence —NR₃₁R₃₂ or 4- to 7-membered heterocyclyl comprising 1-3 heteroatoms selected from N, O, and S optionally substituted with one to three R₃₀. In another embodiment, each R₂₉ is independently at each occurrence —NR₃₁R₃₂. In yet another embodiment, each R₂₉ is independently at each occurrence —NR₃₁R₃₂ or 5- to 7-membered heterocyclyl comprising 1-3 heteroatoms selected from N, O, and S optionally substituted with one to three R₃₀. In another embodiment, each R₂₉ is independently at each occurrence —NR₃₁R₃₂ or 6- or 7-membered heterocyclyl comprising 1-3 heteroatoms selected from N, O, and S optionally substituted with one to three R₃₀. In yet another embodiment, each R₂₉ is independently at each occurrence —NR₃₁R₃₂ or 4- to 6-membered heterocyclyl comprising 1-3 heteroatoms selected from N, O, and S optionally substituted with one to three R₃₀. In another embodiment, each R₂₉ is independently at each occurrence —NR₃₁R₃₂ or 4- or 5-membered heterocyclyl comprising 1-3 heteroatoms selected from N, O, and S optionally substituted with one to three R₃₀.

In another embodiment, each R₂₉ is independently at each occurrence 4-membered heterocyclyl comprising 1-3 heteroatoms selected from N, O, and S optionally substituted with one to three R₃₀. In yet another embodiment, each R₂₉ is independently at each occurrence 5-membered heterocyclyl comprising 1-3 heteroatoms selected from N, O, and S optionally substituted with one to three R₃₀. In another embodiment, each R₂₉ is independently at each occurrence 6-membered heterocyclyl comprising 1-3 heteroatoms selected from N, O, and S optionally substituted with one to three R₃₀.

In another embodiment, each R₂₉ is independently at each occurrence 4- to 7-membered heterocyclyl comprising 1-3 heteroatoms selected from N, O, and S optionally substituted with one to three R₃₀. In yet another embodiment, each R₂₉ is independently at each occurrence 5- to 7-membered heterocyclyl comprising 1-3 heteroatoms selected from N, O, and S optionally substituted with one to three R₃₀. In another embodiment, each R₂₉ is independently at each occurrence 6- or 7-membered heterocyclyl comprising 1-3 heteroatoms selected from N, O, and S optionally substituted with one to three R₃₀. In yet another embodiment, each R₂₉ is independently at each occurrence 4- or 5-membered heterocyclyl comprising 1-3 heteroatoms selected from N, O, and S optionally substituted with one to three R₃₀. In another embodiment, each R₂₉ is independently at each occurrence 4- to 6-membered heterocyclyl comprising 1-3 heteroatoms selected from N, O, and S optionally substituted with one to three R₃₀.

In some embodiments of the Formulae above, each R₃₀ is independently at each occurrence —OH, halogen, (C₁-C₆)alkyl, or (C₁-C₆)haloalkyl. In another embodiment, each R₃₀ is independently at each occurrence halogen, (C₁-C₆)alkyl, or (C₁-C₆)haloalkyl. In another embodiment, each R₃₀ is independently at each occurrence —OH, (C₁-C₆)alkyl, or (C₁-C₆)haloalkyl. In another embodiment, each R₃₀ is independently at each occurrence —OH, halogen, or (C₁-C₆)alkyl. In another embodiment, each R₃₀ is independently at each occurrence —OH, halogen, or (C₁-C₆)haloalkyl.

In some embodiments of the Formulae above, two R₃₀, when on the same atom, together with the atom to which they are attached form a (C₃-C₆)spirocycloalkyl or 4- to 7-membered spiroheterocyclyl ring comprising 1-3 heteroatoms selected from N, O, and S. In another embodiment, two R₃₀, when on the same atom, together with the atom to which they are attached form a (C₃-C₇)spirocycloalkyl or 4- to 6-membered spiroheterocyclyl ring comprising 1-3 heteroatoms selected from N, O, and S. In yet another embodiment, two R₃₀, when on the same atom, together with the atom to which they are attached form a (C₃-C₅)spirocycloalkyl or 4- or 5-membered spiroheterocyclyl ring comprising 1-3 heteroatoms selected from N, O, and S. In another embodiment, two R₃₀, when on the same atom, together with the atom to which they are attached form a (C₃-C₇)spirocycloalkyl.

In another embodiment, two R₃₀, when on the same atom, together with the atom to which they are attached form a (C₃-C₆)spirocycloalkyl. In another embodiment, two R₃₀, when on the same atom, together with the atom to which they are attached form a (C₃-C₅)spirocycloalkyl. In another embodiment, two R₃₀, when on the same atom, together with the atom to which they are attached form a (C₃-C₄)spirocycloalkyl. In another embodiment, two R₃₀, when on the same atom, together with the atom to which they are attached form a (C₄-C₇)spirocycloalkyl. In yet another embodiment, two R₃₀, when on the same atom, together with the atom to which they are attached form a (C₅-C₇)spirocycloalkyl. In another embodiment, two R₃₀, when on the same atom, together with the atom to which they are attached form a (C₆-C₇)spirocycloalkyl.

In another embodiment, two R₃₀, when on the same atom, together with the atom to which they are attached form a 4- to 7-membered spiroheterocyclyl ring comprising 1-3 heteroatoms selected from N, O, and S. In yet another embodiment, two R₃₀, when on the same atom, together with the atom to which they are attached form a 4- to 6-membered spiroheterocyclyl ring comprising 1-3 heteroatoms selected from N, O, and S. In another embodiment, two R₃₀, when on the same atom, together with the atom to which they are attached form a 4- to 5-membered spiroheterocyclyl ring comprising 1-3 heteroatoms selected from N, O, and S. In yet another embodiment, two R₃₀, when on the same atom, together with the atom to which they are attached form a 5- to 7-membered spiroheterocyclyl ring comprising 1-3 heteroatoms selected from N, O, and S. In another embodiment, two R₃₀, when on the same atom, together with the atom to which they are attached form a 6- to 7-membered spiroheterocyclyl ring comprising 1-3 heteroatoms selected from N, O, and S.

In some embodiments of the Formulae above, R₃₁ is H, (C₁-C₆)alkyl, (C₁-C₆)hydroxyalkyl, (C₃-C₇) cycloalkyl, or 4- to 7-membered heterocyclyl comprising 1-3 heteroatoms selected from N, O, and S, wherein the alkyl is optionally substituted with one to ten D atoms, and the cycloalkyl and heterocyclyl are optionally substituted with one to three substituents each independently selected from (C₁-C₆)alkyl, (C₁-C₆)haloalkyl, halogen, and —OH. In another embodiment, R₃₁ is H, (C₁-C₆)alkyl, (C₁-C₆)hydroxyalkyl, or 4- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH.

In another embodiment, R₃₁ is H, (C₁-C₆)alkyl, (C₁-C₆)hydroxyalkyl, or 5- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In yet another embodiment, R₃₁ is H, (C₁-C₆)alkyl, (C₁-C₆)hydroxyalkyl, or 4- to 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In another embodiment, R₃₁ is H, (C₁-C₆)alkyl, (C₁-C₆)hydroxyalkyl, or 5- or 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH.

In another embodiment, R₃₁ is H, (C₁-C₆)alkyl, (C₁-C₆)hydroxyalkyl, or 4- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In yet another embodiment, R₃₁ is H, (C₁-C₆)alkyl, (C₁-C₆)hydroxyalkyl, or 5- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In another embodiment, R₃₁ is H, (C₁-C₆)alkyl, (C₁-C₆)hydroxyalkyl, or 4- to 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In yet another embodiment, R₃₁ is H, (C₁-C₆)alkyl, (C₁-C₆)hydroxyalkyl, or 5- or 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl.

In another embodiment, R₃₁ is H, (C₁-C₆)alkyl, or 4- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In yet another embodiment, R₃₁ is H, (C₁-C₆)alkyl, or 5- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In another embodiment, R₃₁ is H, (C₁-C₆)alkyl, or 4- to 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In yet another embodiment, R₃₁ is H, (C₁-C₆)alkyl, or 5- or 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH.

In another embodiment, R₃₁ is H, (C₁-C₆)alkyl, or 4- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In yet another embodiment, R₃₁ is H, (C₁-C₆)alkyl, or 5- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In another embodiment, R₃₁ is H, (C₁-C₆)alkyl, or 4- to 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In yet another embodiment, R₃₁ is H, (C₁-C₆)alkyl, or 5- or 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl.

In some embodiments of the Formulae above, R₃₂ is H, (C₁-C₆)alkyl, (C₁-C₆)hydroxyalkyl, (C₃-C₇) cycloalkyl, or 4- to 7-membered heterocyclyl comprising 1-3 heteroatoms selected from N, O, and S, wherein the alkyl is optionally substituted with one to ten D atoms, and the cycloalkyl and heterocyclyl are optionally substituted with one to three substituents each independently selected from (C₁-C₆)alkyl, (C₁-C₆)haloalkyl, halogen, and —OH. In another embodiment, R₃₂ is H, (C₁-C₆)alkyl, (C₁-C₆)hydroxyalkyl, or 4- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH.

In another embodiment, R₃₂ is H, (C₁-C₆)alkyl, (C₁-C₆)hydroxyalkyl, or 5- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In yet another embodiment, R₃₂ is H, (C₁-C₆)alkyl, (C₁-C₆)hydroxyalkyl, or 4- to 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In another embodiment, R₃₂ is H, (C₁-C₆)alkyl, (C₁-C₆)hydroxyalkyl, or 5- or 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH.

In another embodiment, R₃₂ is H, (C₁-C₆)alkyl, (C₁-C₆)hydroxyalkyl, or 4- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In yet another embodiment, R₃₂ is H, (C₁-C₆)alkyl, (C₁-C₆)hydroxyalkyl, or 5- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In another embodiment, R₃₂ is H, (C₁-C₆)alkyl, (C₁-C₆)hydroxyalkyl, or 4- to 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In yet another embodiment, R₃₂ is H, (C₁-C₆)alkyl, (C₁—C₆)hydroxyalkyl, or 5- or 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl.

In another embodiment, R₃₂ is H, (C₁-C₆)alkyl, or 4- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In yet another embodiment, R₃₂ is H, (C₁-C₆)alkyl, or 5- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In another embodiment, R₃₂ is H, (C₁-C₆)alkyl, or 4- to 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH. In yet another embodiment, R₃₂ is H, (C₁-C₆)alkyl, or 5- or 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl, (C₁-C₆)haloalkyl, halogen, and —OH.

In another embodiment, R₃₂ is H, (C₁-C₆)alkyl, or 4- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In yet another embodiment, R₃₂ is H, (C₁-C₆)alkyl, or 5- to 7-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In another embodiment, R₃₂ is H, (C₁-C₆)alkyl, or 4- to 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl. In yet another embodiment, R₃₂ is H, (C₁-C₆)alkyl, or 5- or 6-membered heterocyclyl comprising 1-3 heteroatoms selected from N, O, and S, wherein the alkyl is optionally substituted with one to ten D atoms, and the heterocyclyl is optionally substituted with one to three substituents each independently selected from (C₁-C₆)alkyl.

In some embodiments of the Formulae above, each R₃₃ is independently at each occurrence (C₁-C₆)alkyl, (C₁-C₆)haloalkyl, or —C(O)R, wherein R is (C₁-C₆)haloalkyl, (C₃-C₇)cycloalkyl, 4- to 7-membered heterocyclyl comprising 1-3 heteroatoms selected from N, O, and S, or (C₁-C₆)alkyl optionally substituted with one to three (C₁-C₆)alkoxy. In another embodiment, each R₃₃ is independently at each occurrence (C₁-C₆)alkyl or —C(O)R, wherein R is (C₁-C₆)haloalkyl, (C₃-C₇)cycloalkyl, 4- to 7-membered heterocyclyl comprising 1-3 heteroatoms selected from N, O, and S, or (C₁-C₆)alkyl optionally substituted with one to three (C₁-C₆)alkoxy. In yet another embodiment, each R₃₃ is independently at each occurrence (C₁-C₆)alkyl, (C₁-C₆)haloalkyl, or —C(O)R, wherein R is (C₃-C₇)cycloalkyl, 4- to 7-membered heterocyclyl comprising 1-3 heteroatoms selected from N, O, and S, or (C₁-C₆)alkyl optionally substituted with one to three (C₁-C₆)alkoxy.

In another embodiment, each R₃₃ is independently at each occurrence (C₁-C₆)alkyl, (C₁-C₆)haloalkyl, or —C(O)R, wherein R is (C₁-C₆)haloalkyl, 4- to 7-membered heterocyclyl comprising 1-3 heteroatoms selected from N, O, and S, or (C₁-C₆)alkyl optionally substituted with one to three (C₁-C₆)alkoxy. In yet another embodiment, each R₃₃ is independently at each occurrence (C₁-C₆)alkyl, (C₁-C₆)haloalkyl, or —C(O)R, wherein R is 4- to 7-membered heterocyclyl comprising 1-3 heteroatoms selected from N, O, and S, or (C₁-C₆)alkyl optionally substituted with one to three (C₁-C₆)alkoxy. In another embodiment, each R₃₃ is independently at each occurrence (C₁-C₆)alkyl or —C(O)R, wherein R is 4- to 7-membered heterocyclyl comprising 1-3 heteroatoms selected from N, O, and S, or (C₁-C₆)alkyl optionally substituted with one to three (C₁-C₆)alkoxy.

In another embodiment, each R₃₃ is independently at each occurrence (C₁-C₆)alkyl or —C(O)R, wherein R is 5- to 7-membered heterocyclyl comprising 1-3 heteroatoms selected from N, O, and S, or (C₁-C₆)alkyl optionally substituted with one to three (C₁-C₆)alkoxy. In yet another embodiment, each R₃₃ is independently at each occurrence (C₁-C₆)alkyl or —C(O)R, wherein R is 4- to 6-membered heterocyclyl comprising 1-3 heteroatoms selected from N, O, and S, or (C₁-C₆)alkyl optionally substituted with one to three (C₁-C₆)alkoxy. In another embodiment, each R₃₃ is independently at each occurrence (C₁-C₆)alkyl or —C(O)R, wherein R is 5- or 6-membered heterocyclyl comprising 1-3 heteroatoms selected from N, O, and S, or (C₁-C₆)alkyl optionally substituted with one to three (C₁-C₆)alkoxy.

In some embodiments of the Formulae above, two R₃₃, when on the same atom, together with the atom to which they are attached form a (C₃-C₇)spirocycloalkyl or 4- to 7-membered spiroheterocyclyl ring comprising 1-3 heteroatoms selected from N, O, and S. In yet another embodiment, two R₃₃, when on the same atom, together with the atom to which they are attached form a (C₃-C₆)spirocycloalkyl or 4- to 6-membered spiroheterocyclyl ring comprising 1-3 heteroatoms selected from N, O, and S. In another embodiment, two R₃₃, when on the same atom, together with the atom to which they are attached form a (C₃-C₆)spirocycloalkyl or 4- or 5-membered spiroheterocyclyl ring comprising 1-3 heteroatoms selected from N, O, and S. In yet another embodiment, two R₃₃, when on the same atom, together with the atom to which they are attached form a (C₃-C₄)spirocycloalkyl or 4- to 7-membered spiroheterocyclyl ring comprising 1-3 heteroatoms selected from N, O, and S.

In another embodiment, two R₃₃, when on the same atom, together with the atom to which they are attached form a (C₄-C₇)spirocycloalkyl or 5- to 7-membered spiroheterocyclyl ring comprising 1-3 heteroatoms selected from N, O, and S. In yet another embodiment, two R₃₃, when on the same atom, together with the atom to which they are attached form a (C₅-C₇)spirocycloalkyl or 6- or 7-membered spiroheterocyclyl ring comprising 1-3 heteroatoms selected from N, O, and S. In another embodiment, two R₃₃, when on the same atom, together with the atom to which they are attached form a (C₃-C₇)spirocycloalkyl. In yet another embodiment, two R₃₃, when on the same atom, together with the atom to which they are attached form a (C₃-C₆)spirocycloalkyl. In another embodiment, two R₃₃, when on the same atom, together with the atom to which they are attached form a (C₃-C₆)spirocycloalkyl. In yet another embodiment, two R₃₃, when on the same atom, together with the atom to which they are attached form a (C₄-C₇)spirocycloalkyl. In another embodiment, two R₃₃, when on the same atom, together with the atom to which they are attached form a (C₅-C₇)spirocycloalkyl. In yet another embodiment, two R₃₃, when on the same atom, together with the atom to which they are attached form a (C₆-C₇)spirocycloalkyl.

In another embodiment, two R₃₃, when on the same atom, together with the atom to which they are attached form a 4- to 7-membered spiroheterocyclyl ring comprising 1-3 heteroatoms selected from N, O, and S. In yet another embodiment, two R₃₃, when on the same atom, together with the atom to which they are attached form a 5- to 7-membered spiroheterocyclyl ring comprising 1-3 heteroatoms selected from N, O, and S. In another embodiment, two R₃₃, when on the same atom, together with the atom to which they are attached form a 6- or 7-membered spiroheterocy

In some embodiments of the Formulae above, each R₃₄ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with (C₁-C₆)alkyl optionally substituted with one to three substituents each independently selected from (C₃-C₇)cycloalkyl and 4- to 10-membered mono or bicyclic heterocyclyl comprising 1-4 heteroatoms selected from N, O, and S. In another embodiment, each R₃₄ is independently at each occurrence (C₃-C₇)cycloalkyl optionally substituted with (C₁-C₆)alkyl optionally substituted with one to three substituents each independently selected from (C₃-C₇)cycloalkyl and 4- to 10-membered mono or bicyclic heterocyclyl comprising 1-4 heteroatoms selected from N, O, and S.

In another embodiment, each R₃₄ is independently at each occurrence 4- to 10-membered mono or bicyclic heterocyclyl comprising 1-4 heteroatoms selected from N, O, and S, optionally substituted with (C₁-C₆)alkyl optionally substituted with one to three substituents each independently selected from (C₃-C₇)cycloalkyl and 4- to 10-membered mono or bicyclic heterocyclyl comprising 1-4 heteroatoms selected from N, O, and S. In another embodiment, each R₃₄ is independently at each occurrence 4- to 10-membered mono or bicyclic heterocyclyl comprising 1-4 heteroatoms selected from N, O, and S, optionally substituted with (C₁-C₆)alkyl optionally substituted with one to three (C₃-C₇)cycloalkyl.

In some embodiments of the Formulae above, m is 1-13. In another embodiment, m is 1-12. In another embodiment, m is 1-11. In another embodiment, m is 1-10. In another embodiment, m is 1-9. In another embodiment, m is 1-8. In another embodiment, m is 1-7. In another embodiment, m is 1-6. In another embodiment, m is 1-5. In another embodiment, m is 1-4. In another embodiment, m is 1-3. In another embodiment, m is 1-2. In another embodiment, m is 2-13. In another embodiment, m is 3-13. In another embodiment, m is 4-13. In another embodiment, m is 5-13. In another embodiment, m is 6-13. In another embodiment, m is 7-13. In another embodiment, m is 8-13. In another embodiment, m is 9-13. In another embodiment, m is 10-13. In another embodiment, m is 11-13. In another embodiment, m is 12-13.

In some embodiments of the Formulae above, n is 1, 2, 3, or 4. In another embodiment, n is 1, 2, or 3. In yet another embodiment, n is 2, 3, or 4. In another embodiment, n is 1 or 2. In yet another embodiment, n is 2 or 3. In another embodiment, n is 3 or 4. In yet another embodiment, n is 1. In another embodiment, n is 2. In yet another embodiment, n is 3. In another embodiment, n is 4.

In some embodiments of the Formulae above, R₁ is H or (C₁-C₆)alkyl; or R₁ and R₁₁ together with the atoms to which they are attached form a 5- to 7-membered heterocyclyl ring comprising 1-3 heteroatoms selected from N, O, and S optionally substituted with ═(O).

In some embodiments of the Formulae above, R₁₀ is phenyl substituted with —OR₁₃ and optionally substituted with one to three R₁₄. In another embodiment, R₁₀ is pyridinyl or pyridinone substituted with —OR₁₃ and optionally substituted with one to three R₁₄.

In some embodiments of the Formulae above, R₁₃ is phenyl substituted with R₁₆ and optionally substituted with one to three R_{16′}. In another embodiment, R₁₃ is pyridinyl substituted with R₁₆ and optionally substituted with one to three R_{16′}.

In some embodiments of the Formulae above, q is 0, 1, or 2. In another embodiment, q is 0 or 1. In yet another embodiment, q is 1 or 2. In another embodiment, q is 0 or 2. In yet another embodiment, q is 0. In another embodiment, q is 1. In yet another embodiment, q is 2.

In some embodiments of the Formulae above, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), and X₁ is C. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, and R₃ is H or (C₁-C₆)alkyl. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, and R₄ is H. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, and R₅ is H. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, and R₆ is H or (C₁-C₆)alkyl.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, and R_{7′} is H or (C₁-C₆)alkyl. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl and R₈ is (C₁-C₆)alkyl. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, and R₁₂ is halogen.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, and n is 1. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, n is 1, and R₁₀ is phenyl substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is (C₁-C₆)alkyl, R₁₂ is halogen, n is 1, R₁₀ is phenyl substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, n is 1, R₁₀ is phenyl substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is (C₁-C₆)alkyl, R₁₂ is halogen, n is 1, and R₁₀ is pyridinyl or pyridinone substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is (C₁-C₆)alkyl, R₁₂ is halogen, n is 1, R₁₀ is pyridinyl or pyridinone substituted with —OR₁₃ and optionally substituted with one to three R₁₄ and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is (C₁-C₆)alkyl, R₁₂ is halogen, n is 1, R₁₀ is pyridinyl or pyridinone substituted with —OR₁₃ and optionally substituted with one to three R₁₄ and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is (C₁-C₆)alkyl, R₁₂ is halogen, and n is 2. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, n is 2, and R₁₀ is phenyl substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, n is 2, R₁₀ is phenyl substituted with —OR₁₃ and optionally substituted with one to three R₁₄, and R₁₃ is phenyl substituted with R₁₆ and optionally substituted with one to three R_{16′}. In yet another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, n is 2, R₁₀ is phenyl substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, n is 2, and R₁₀ is pyridinyl or pyridinone substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, n is 2, R₁₀ is pyridinyl or pyridinone substituted with —OR₁₃ and optionally substituted with one to three R₁₄, and R₁₃ is phenyl substituted with R₁₆ and optionally substituted with one to three R_{16′}. In yet another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is halogen, n is 2, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl and R₈ is (C₁-C₆)alkyl. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, and R₁₂ is chloro or fluoro.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, and n is 1. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 1, and R₁₀ is phenyl substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 1, R₁₀ is phenyl substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 1, R₁₀ is phenyl substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 1, and R₁₀ is pyridinyl or pyridinone substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro n is 1, R₁₀ is pyridinyl or pyridinone substituted with —OR₁₃ and optionally substituted with one to three R₁₄, and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 1, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro and n is 2. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 2, and R₁₀ is phenyl substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 2, R₁₀ is phenyl substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 2, R₁₀ is phenyl substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 2, and R₁₀ is pyridinyl or pyridinone substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro, n is 2, R₁₀ is pyridinyl or pyridinone substituted with —OR₁₃ and optionally substituted with one to three R₁₄, and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl R₈ is (C₁-C₆)alkyl, R₁₂ is chloro or fluoro n is 2, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, and R₈ is methyl. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, and R₁₂ is halogen. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, and n is 1.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 1, and R₁₀ is phenyl substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 1, R₁₀ is phenyl substituted with —OR₁₃ and optionally substituted with one to three R₁₄, and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 1, R₁₀ is phenyl substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 1, and R₁₀ is pyridinyl or pyridinone substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 1, R₁₀ is pyridinyl or pyridinone substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 1, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, and n is 2. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 2, and R₁₀ is phenyl substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 2, R₁₀ is phenyl substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 2, R₁₀ is phenyl substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 2, and R₁₀ is pyridinyl or pyridinone substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 2, R₁₀ is pyridinyl or pyridinone substituted with —OR₁₃ and optionally substituted with one to three R₁₄, and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is halogen, n is 2, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, and R₁₂ is chloro or fluoro. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is chloro or fluoro, and n is 1.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is chloro or fluoro, n is 1, and R₁₀ is phenyl substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is chloro or fluoro, n is 1, R₁₀ is phenyl substituted with —OR₁₃ and optionally substituted with one to three R₁₄, and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is chloro or fluoro, n is 1, R₁₀ is phenyl substituted with —OR₁₃ and optionally substituted with one to three R₁₄, and R₁₃ is pyridinyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is chloro or fluoro, n is 1, and R₁₀ is pyridinyl or pyridinone substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is chloro or fluoro, n is 1, R₁₀ is pyridinyl or pyridinone substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H or (C₁-C₆)alkyl, R₈ is methyl, R₁₂ is chloro or fluoro, n is 1, R₁₀ is pyridinyl or pyridinone substituted with —OR₁₃ and optionally substituted with one to three R₁₄, and wherein R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, and R_{7′} is H.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, and R₈ is (C₁-C₆)alkyl. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, and R₁₂ is halogen. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, and n is 1.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 1, and R₁₀ is phenyl substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 1, R₁₀ is phenyl substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 1, R₁₀ is phenyl substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 1, and R₁₀ is pyridinyl or pyridinone substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 1, R₁₀ is pyridinyl or pyridinone substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 1, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, and n is 2. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 2, and R₁₀ is phenyl substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 2, R₁₀ is phenyl substituted with —OR₁₃ and optionally substituted with one to three R₁₄, and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 2, R₁₀ is phenyl substituted with —OR₁₃ and optionally substituted with one to three R₁₄, and R₁₃ is pyridinyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 2, and R₁₀ is pyridinyl or pyridinone substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 2, R₁₀ is pyridinyl or pyridinone substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is halogen, n is 2, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, and R₁₂ is chloro or fluoro. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is chloro or fluoro, and n is 1.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is chloro or fluoro, n is 1, and R₁₀ is phenyl substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is chloro or fluoro, n is 1, R₁₀ is phenyl substituted with —OR₁₃ and optionally substituted with one to three R₁₄, and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, R₈ is (C₁-C₆)alkyl, R₁₂ is chloro or fluoro, n is 1, R₁₀ is phenyl substituted with —OR₁₃ and optionally substituted with one to three R₁₄, and R₁₃ is pyridinyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 1, and R₁₀ is pyridinyl or pyridinone substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 1, R₁₀ is pyridinyl or pyridinone substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 1, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, and n is 2. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 2, and R₁₀ is phenyl substituted with —OR₁₃ and optionally substituted with one to three R₁₄. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 2, R₁₀ is phenyl substituted with —OR₁₃ and optionally substituted with one to three R₁₄, and R₁₃ is phenyl substituted with R₁₆ and optionally substituted with one to three R_{16′}.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 2, R₁₀ is phenyl substituted with —OR₁₃ and optionally substituted with one to three R₁₄, and R₁₃ is pyridinyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 2, and R₁₀ is pyridinyl or pyridinone substituted with —OR₁₃ and optionally substituted with one to three R₁₄.

In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 2, R₁₀ is pyridinyl or pyridinone substituted with —OR₁₃ and optionally substituted with one to three R₁₄, and R₁₃ is phenyl substituted with R₁₆ and optionally substituted with one to three R_{16′}. In another embodiment, R₁ is H or (C₁-C₆)alkyl, or R₁ and R₁₁ together with the atoms to which they are attached form a 5- to 7-membered heterocyclyl ring comprising 1-3 heteroatoms selected from N, O, and S optionally substituted with ═(O), X₁ is C, R₃ is H or (C₁-C₆)alkyl, R₄ is H, R₅ is H, R₆ is H or (C₁-C₆)alkyl, R_{7′} is H, Rₐ is (C₁-C₆)alkyl, R₁₂ is chloro or fluoro, n is 2, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In some embodiments of the Formulae above, X₁ is C, and R₁ is H or (C₁-C₆)alkyl. In another embodiment, X₁ is C, R₁ is H or (C₁-C₆)alkyl and R₃ is H or (C₁-C₆)alkyl. In another embodiment, X₁ is C, R₁ is H or (C₁-C₆)alkyl, R₃ is H or (C₁-C₆)alkyl, and R₄ is H. In another embodiment, X₁ is C, R₁ is H or (C₁-C₆)alkyl, R₃ is H or (C₁-C₆)alkyl, R₄ is H, and R₅ is H. In another embodiment, X₁ is C, R₁ is H or (C₁-C₆)alkyl, R₃ is H or (C₁-C₆)alkyl, R₄ is H, R₅ is H, and R_{6′} is H or (C₁-C₆)alkyl. In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, and R_{7′} is H or (C₁-C₆)alkyl. In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, and R₈ is (C₁-C₆)alkyl. In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, R₃ is (C₁-C₆)alkyl, and R₁₂ is halogen. In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, R₈ is (C₁-C₆)alkyl, R₁₂ is halogen, and n is 1.

In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, Rₐ is (C₁-C₆)alkyl, R₁₂ is halogen, n is 1, and R₁₀ is phenyl substituted with —OR₁₃ and optionally substituted with one to three R₁₄. In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, Rₐ is (C₁-C₆)alkyl, R₁₂ is halogen, n is 1, R₁₀ is phenyl substituted with —OR₁₃ and optionally substituted with one to three R₁₄, and R₁₃ is phenyl substituted with R₁₆ and optionally substituted with one to three R_{16′}.

In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, Rₐ is (C₁-C₆)alkyl, R₁₂ is halogen, n is 1, R₁₀ is phenyl substituted with —OR₁₃ and optionally substituted with one to three R₁₄, and R₁₃ is pyridinyl substituted with R₁₆ and optionally substituted with one to three R_{16′}. In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, Rₐ is (C₁-C₆)alkyl, R₁₂ is halogen, n is 1, and R₁₀ is pyridinyl or pyridinone substituted with —OR₁₃ and optionally substituted with one to three R₁₄. In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, Rₐ is (C₁-C₆)alkyl, R₁₂ is halogen, n is 1, R₁₀ is pyridinyl or pyridinone substituted with —OR₁₃ and optionally substituted with one to three R₁₄, and R₁₃ is phenyl substituted with R₁₆ and optionally substituted with one to three R_{16′}. In another embodiment, X₁ is C, R₁ is H or (C₁-C₆)alkyl, R₃ is H or (C₁-C₆)alkyl, R₄ is H, R₅ is H, R_{6′} is H or (C₁-C₆)alkyl, R_{7′} is H or (C₁-C₆)alkyl, Rₐ is (C₁-C₆)alkyl, R₁₂ is halogen, n is 1, R₁₀ is pyridinyl or pyridinone substituted with —OR₁₃ and optionally substituted with one to three R₁₄, and R₁₃ is pyridinyl substituted with R₁₆ and optionally substituted with one to three R_{16′}.

In some embodiments of the Formulae above, X₁ is C, R₁ is H or (C₁-C₆)alkyl and R₃ is H or methyl. In another embodiment, X₁ is C, R₁ is H or (C₁-C₆)alkyl, R₃ is H or methyl, and R₄ is H. In another embodiment, X₁ is C, R₁ is H or (C₁-C₆)alkyl, R₃ is H or methyl, R₄ is H, and R₅ is H. In another embodiment, X₁ is C, R₁ is H or (C₁-C₆)alkyl, R₃ is H or methyl, R₄ is H, R₅ is H, and R_{6′} is H or methyl. In another embodiment, X₁ is C, R₁ is H or (C₁-C₆)alkyl, R₃ is H or methyl, R₄ is H, R₅ is H, R_{6′} is H or methyl, and R_{7′} is H. In another embodiment, X₁ is C, R₁ is H or (C₁-C₆)alkyl, R₃ is H or methyl, R₄ is H, R₅ is H, R_{6′} is H or methyl, R_{7′} is H, and Rₐ is methyl. In another embodiment, X₁ is C, R₁ is H or (C₁-C₆)alkyl, R₃ is H or methyl, R₄ is H, R₅ is H, R_{6′} is H or methyl, R_{7′} is H, R₈ is methyl, and R₁₂ is chloro or fluoro. In another embodiment, X₁ is C, R₁ is H or (C₁-C₆)alkyl, R₃ is H or methyl, R₄ is H, R₅ is H, R_{6′} is H or methyl, R_{7′} is H, R₈ is methyl, R₁₂ is chloro or fluoro, and n is 1.

In another embodiment, X₁ is C, R₁ is H or (C₁-C₆)alkyl, R₃ is H or methyl, R₄ is H, R₅ is H, R_{6′} is H or methyl, R_{7′} is H, R₈ is methyl, R₁₂ is chloro or fluoro, n is 1, and R₁₀ is phenyl substituted with —OR₁₃ and optionally substituted with one to three R₁₄. In another embodiment, X₁ is C, R₁ is H or (C₁-C₆)alkyl, R₃ is H or methyl, R₄ is H, R₅ is H, R_{6′} is H or methyl, R_{7′} is H, R₈ is methyl, R₁₂ is chloro or fluoro, n is 1, R₁₀ is phenyl substituted with —OR₁₃ and optionally substituted with one to three R₁₄, and R₁₃ is phenyl substituted with R₁₆ and optionally substituted with one to three R_{16′}.

In another embodiment, X₁ is C, R₁ is H or (C₁-C₆)alkyl, R₃ is H or methyl, R₄ is H, R₅ is H, R_{6′} is H or methyl, R_{7′} is H, R₈ is methyl, R₁₂ is chloro or fluoro, n is 1, R₁₀ is phenyl substituted with —OR₁₃ and optionally substituted with one to three R₁₄, and R₁₃ is pyridinyl substituted with R₁₆ and optionally substituted with one to three R_{16′}. In another embodiment, X₁ is C, R₁ is H or (C₁-C₆)alkyl, R₃ is H or methyl, R₄ is H, R₅ is H, R_{6′} is H or methyl, R_{7′} is H, R₈ is methyl, R₁₂ is chloro or fluoro, n is 1, and R₁₀ is pyridinyl or pyridinone substituted with —OR₁₃ and optionally substituted with one to three R₁₄.

In another embodiment, X₁ is C, R₁ is H or (C₁-C₆)alkyl, R₃ is H or methyl, R₄ is H, R₅ is H, R_{6′} is H or methyl, R_{7′} is H, R₈ is methyl, R₁₂ is chloro or fluoro, n is 1, R₁₀ is pyridinyl or pyridinone substituted with —OR₁₃ and optionally substituted with one to three R₁₄ and R₁₃ is phenyl substituted with R₁₆ and optionally substituted with one to three R_{16′}. In another embodiment, X₁ is C, R₁ is H or (C₁-C₆)alkyl, R₃ is H or methyl, R₄ is H, R₅ is H, R_{6′} is H or methyl, R_{7′} is H, R₈ is methyl, R₁₂ is chloro or fluoro, n is 1, R₁₀ is pyridinyl or pyridinone substituted with —OR₁₃ and optionally substituted with one to three R₁₄ and R₁₃ is pyridinyl substituted with R₁₆ and optionally substituted with one to three R_{16′}.

Embodiment 1. A compound according to Formula (I), wherein X₁, R₁, R₂, R₃, R₄, R₅, R₆, R_{6′}, R₇, R_{7′}, R₈, R₉, R_{9′}, R₁₀, R₁₁, R₁₂, and n are as described herein above.

Embodiment 2. The compound according to Embodiment 1, wherein X₁ is C.

Embodiment 3. The compound according to Embodiment 1 or 2, wherein R₁ is H or (C₁-C₆)alkyl; or R₁ and R₁₁ together with the atoms to which they are attached form a 5- to 7-membered heterocyclyl ring comprising 1-3 heteroatoms selected from N, O, and S optionally substituted with ═(O).

Embodiment 4. The compound according to any one of Embodiments 1-3, wherein R₁ is H.

Embodiment 5. The compound according to any one of Embodiments 1-3, wherein R₁ and R₁₁ together with the atoms to which they are attached form a 5- to 7-membered heterocyclyl ring comprising 1-3 heteroatoms selected from N, O, and S optionally substituted with ═(O).

Embodiment 6. The compound according to any one of Embodiments 1-5, wherein R₃ is H or (C₁-C₆)alkyl.

Embodiment 7. The compound according to any one of Embodiments 1-6, wherein R₃ is H or methyl.

Embodiment 8. The compound according to any one of Embodiments 1-7, wherein R₄ is H.

Embodiment 9. The compound according to any one of Embodiments 1-8, wherein R₅ is H.

Embodiment 10. The compound according to any one of Embodiments 1-9, wherein R₆ is H or (C₁-C₆)alkyl.

Embodiment 11. The compound according to any one of Embodiments 1-10, wherein R₆ is H or methyl.

Embodiment 12. The compound according to any one of Embodiments 1-11, wherein R_{6′} is H or (C₁-C₆)alkyl.

Embodiment 13. The compound according to any one of Embodiments 1-12, wherein R_{6′}, is H or methyl.

Embodiment 14. The compound according to any one of Embodiments 1-13, wherein R_{7′} is H or (C₁-C₆)alkyl.

Embodiment 15. The compound according to any one of Embodiments 1-14, wherein R_{7′} is H.

Embodiment 16. The compound according to any one of Embodiments 1-15, wherein R₈ is (C₁-C₆)alkyl.

Embodiment 17. The compound according to any one of Embodiments 1-16, wherein R₈ is methyl.

Embodiment 18. The compound according to any one of Embodiments 1-17, wherein R₁₂ is halogen.

Embodiment 19. The compound according to any one of Embodiments 1-18, wherein R₁₂ is chloro or fluoro.

Embodiment 20. The compound according to any one of Embodiments 1-19, wherein n is 1.

Embodiment 21. The compound according to any one of Embodiments 1-19, wherein n is 2.

Embodiment 22. The compound according to Embodiment 1, having the Formula (Ia), Formula (Ia-1), Formula (Ia-2), Formula (Ib), Formula (Ic), Formula (Id), Formula (Ie), Formula (If), Formula (Ig), Formula (Ih), Formula (Ii) or Formula (Ij).

Embodiment 23. The compound according to any one of Embodiments 1-22, wherein R₁₀ is phenyl substituted with —OR₁₃ and optionally substituted with one to three R₁₄.

Embodiment 24. The compound according to any one of Embodiments 1-22, wherein R₁₀ is pyridinyl or pyridinone substituted with —OR₁₃ and optionally substituted with one to three R₁₄.

Embodiment 25. The compound according to any one of Embodiments 1-24, wherein R₁₃ is phenyl substituted with R₁₆ and optionally substituted with one to three R_{16′}.

Embodiment 26. The compound according to any one of Embodiments 1-24, wherein R₁₃ is pyridinyl substituted with R₁₆ and optionally substituted with one to three R_{16′}.

Embodiment 27. The compound (Cmpd) of Embodiment 1 selected from:

| Cmpd No. | Chemical name |
|---|---|
| 2 | (4S,7S,10S,14R,16aS,20aS)-14-((1H-pyrazol-1-yl)methyl)-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 3 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(2- |
| | (methylamino)ethyl)hexadecahydrobenzo[l][1,4,7,11,14]pentaazacyclooctadecine- |
| | 2,6,9,12,15(3H)-pentaone; |
| 4 | tert-butyl (3-((2S,5S,8S,13S,16R)-16-benzyl-1-(4-chloro-2-(4-(2- |
| | ((dimethylamino)methyl)-1-methyl-1H-imidazol-5-yl)phenoxy)benzyl)-8-(4- |
| | chlorobenzyl)-5-(methoxymethyl)-7,13,14-trimethyl-3,6,10,15,18-pentaoxo- |
| | 1,4,7,11,14-pentaazacyclooctadecan-2-yl)propyl)carbamate |
| 5 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-13-(cyclopropylmethyl)-5- |
| | (methoxymethyl)-2,7,14,16-tetramethyl-1,4,7,11,14-pentaazacyclooctadecane- |
| | 3,6,10,15,18-pentaone; |
| 7 | (2S,5S,8S,13S,16R)-1-(4-chloro-2-(4-(1-methyl-2-(pyrrolidin-1-ylmethyl)-1H- |
| | imidazol-5-yl)phenoxy)-6-(2,2,2-trifluoroethoxy)benzyl)-8-(4-chlorobenzyl)-5- |
| | (methoxymethyl)-2,7,13,14,16-pentamethyl-1,4,7,11,14-pentaazacyclooctadecane- |
| | 3,6,10,15,18-pentaone; |
| 8 | (4S,7S,10R,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7,10-bis(methoxymethyl)-5,16- |
| | dimethylhexadecahydrobenzo[l][1,4,7,11,14]pentaazacyclooctadecine- |
| | 2,6,9,12,15(3H)-pentaone; |
| 9 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)benzyl)-5- |
| | (methoxymethyl)-2,7,13,14-tetramethyl-1,4,7,11,14-pentaazacyclooctadecane- |
| | 3,6,10,15,18-pentaone; |
| 10 | tert-butyl 3-((2S,5S,8S,13S,16R)-16-benzyl-1-(4-chloro-2-(4-(2- |
| | ((dimethylamino)methyl)-1-methyl-1H-imidazol-5-yl)phenoxy)benzyl)-8-(4- |
| | chlorobenzyl)-5-(methoxymethyl)-7,13,14-trimethyl-3,6,10,15,18-pentaoxo- |
| | 1,4,7,11,14-pentaazacyclooctadecan-2-yl)propanoate |
| 11 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2-(hydroxymethyl)-5- |
| | (methoxymethyl)-7,13,14-trimethyl-1,4,7,11,14-pentaazacyclooctadecane- |
| | 3,6,10,15,18-pentaone; |
| 12 | (2R,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 13 | (2S,5S,10S,13R,17S)-13-benzyl-16-(4-chloro-2-(4-(2-((dimethylamino)methyl)-1- |
| | methyl-1H-imidazol-5-yl)phenoxy)benzyl)-5-(4-chlorobenzyl)-2-(methoxymethyl)- |
| | 4,10,11-trimethyl-1,4,8,11,16-pentaazabicyclo[15.2.1]icosane-3,7,12,15,19,20- |
| | hexaone; |
| 14 | (4S,7S,10S,14R,16aR,20aS)-14-benzyl-11-(4-chloro-2-(4-(1-methyl-2-(pyrrolidin-1- |
| | ylmethyl)-1H-imidazol-5-yl)phenoxy)benzyl)-4-(4-chlorobenzyl)-7-(methoxymethyl)- |
| | 5,10,16-trimethyltetradecahydro-1H-pyrano[3,4- |
| | l][1,4,7,11,14]pentaazacyclooctadecine-2,6,9,12,15(3H)-pentaone; |
| 15 | (4S,7S,10S,14R,16aS,20aS)-14-benzyl-4-(4-chlorobenzyl)-11-(4-(4-(2- |
| | ((dimethylamino)methyl)-1-methyl-1H-imidazol-5-yl)phenoxy)-2-fluorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 16 | (4S,7S,10S,14R,16aS,20aS)-11-(2-(4-(2-(azetidin-1-ylmethyl)-1-methyl-1H- |
| | imidazol-5-yl)phenoxy)-4-chlorobenzyl)-14-benzyl-4-(4-chlorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 17 | (2S,5S,8S,13S,16R)-2-(azetidin-1-ylmethyl)-16-benzyl-1-(4-chloro-2-(4-(2- |
| | ((dimethylamino)methyl)-1-methyl-1H-imidazol-5-yl)phenoxy)benzyl)-8-(4- |
| | chlorobenzyl)-5-(methoxymethyl)-7,13,14-trimethyl-1,4,7,11,14- |
| | pentaazacyclooctadecane-3,6,10,15,18-pentaone; |
| 18 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-14-(isoxazol-3- |
| | ylmethyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 19 | (4S,7S,10S,14R,16aS,20aS)-14-benzyl-11-(4-chloro-2-(4-(5-methyl-4-(pyrrolidin-1- |
| | ylmethyl)-1H-imidazol-1-yl)phenoxy)benzyl)-4-(4-chlorobenzyl)-7-(methoxymethyl)- |
| | 5,10,16-trimethylhexadecahydrobenzo[l][1,4,7,11,14]pentaazacyclooctadecine- |
| | 2,6,9,12,15(3H)-pentaone; |
| 20 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(2-morpholino-2- |
| | oxoethyl)hexadecahydrobenzo[l][1,4,7,11,14]pentaazacyclooctadecine- |
| | 2,6,9,12,15(3H)-pentaone; |
| 21 | (2S,5S,8S,13S,16R)-16-benzyl-1-(4-chloro-2-(4-(2-(1-hydroxyethyl)-1-methyl-1H- |
| | imidazol-5-yl)phenoxy)benzyl)-8-(4-chlorobenzyl)-5-(hydroxymethyl)-2,7,13,14- |
| | tetramethyl-1,4,7,11,14-pentaazacyclooctadecane-3,6,10,15,18-pentaone; |
| 22 | (2S,5S,8S,13S,16R)-1-(4-chloro-2-(4-(1-methyl-2-(pyrrolidin-1-ylmethyl)-1H- |
| | imidazol-5-yl)phenoxy)benzyl)-8-(4-chlorobenzyl)-16-(2,4-difluorobenzyl)-5- |
| | (methoxymethyl)-2,7,13,14-tetramethyl-1,4,7,11,14-pentaazacyclooctadecane- |
| | 3,6,10,15,18-pentaone; |
| 23 | (4S,7S,10S,14R,16aS,20aS)-11-(2-(4-(2-(2-oxa-6-azaspiro[3.3]heptan-6-ylmethyl)- |
| | 1-methyl-1H-imidazol-5-yl)phenoxy)-4-ethylbenzyl)-14-benzyl-4-(4-chlorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 25 | (2S,5R,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-5-(fluoromethyl)-2,13,14- |
| | trimethyl-1,4,7,11,14-pentaazacyclooctadecane-3,6,10,15,18-pentaone; |
| 26 | (2S,5S,8S,13S,16R)-16-benzyl-8-(4-chlorobenzyl)-1-(4-ethyl-2-(4-(1-methyl-2- |
| | (pyrrolidin-1-ylmethyl)-1H-imidazol-5-yl)phenoxy)benzyl)-2-(2-fluoroethyl)-5- |
| | (methoxymethyl)-7,13,14-trimethyl-1,4,7,11,14-pentaazacyclooctadecane- |
| | 3,6,10,15,18-pentaone; |
| 27 | (2S,5S,8S,13R,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5,13- |
| | bis(methoxymethyl)-2,7,14-trimethyl-1,4,7,11,14-pentaazacyclooctadecane- |
| | 3,6,10,15,18-pentaone; |
| 28 | (2S,5S,8S,13S,16R)-16-benzyl-1-(4-chloro-2-(4-(1-methyl-2-(1-methylpyrrolidin-2- |
| | yl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 29 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-10-(2-(dimethylamino)ethyl)-7-(methoxymethyl)-5,16- |
| | dimethylhexadecahydrobenzo[l][1,4,7,11,14]pentaazacyclooctadecine- |
| | 2,6,9,12,15(3H)-pentaone; |
| 30 | (2S,5S,8S,13S,16R)-16-benzyl-1-(4-chloro-2-(4-(1-methyl-2-(pent-1-yn-1-yl)-1H- |
| | imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 31 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2,7,13,14-tetramethyl- |
| | 5-(oxetan-3-yl)-1,4,7,11,14-pentaazacyclooctadecane-3,6,10,15,18-pentaone; |
| 32 | (2S,5S,8S,12S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 2,7,12,14-tetramethyl-1,4,7,11,14-pentaazacyclooctadecane-3,6,10,15,18- |
| | pentaone; |
| 33 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,13,14- |
| | tetramethyl-16-(2,3,4-trifluorobenzyl)-1,4,7,11,14-pentaazacyclooctadecane- |
| | 3,6,10,15,18-pentaone; |
| 34 | (2S,5S,8S,13S,16R)-16-benzyl-1-(4-chloro-2-fluoro-6-(4-(1-methyl-2-(pyrrolidin-1- |
| | ylmethyl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 35 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)-4- |
| | (trifluoromethoxy)benzyl)-5-(methoxymethyl)-2,7,13,14-tetramethyl-1,4,7,11,14- |
| | pentaazacyclooctadecane-3,6,10,15,18-pentaone; |
| 36 | (2S,5S,8S,13S,16R)-16-benzyl-1-(4-chloro-2-(4-(5-(4-((2- |
| | fluoroethyl)amino)cyclohexyl)pyridin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 37 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,13,14-trimethyl-2-(2,2,2-trifluoroethyl)-1,4,7,11,14-pentaazacyclooctadecane- |
| | 3,6,10,15,18-pentaone; |
| 38 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,13,14-trimethyl-2-(2-morpholino-2-oxoethyl)-1,4,7,11,14- |
| | pentaazacyclooctadecane-3,6,10,15,18-pentaone; |
| 39 | (4S,7S,10S,16aS,20aS)-11-(4-chloro-2-(4-(2-((dimethylamino)methyl)-1-methyl- |
| | 1H-imidazol-5-yl)phenoxy)-6-(2,2,2-trifluoroethoxy)benzyl)-4-(4-chlorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 40 | (2S,5S,8S,13S,16S)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,13,14- |
| | tetramethyl-16-(pentan-3-yl)-1,4,7,11,14-pentaazacyclooctadecane-3,6,10,15,18- |
| | pentaone; |
| 41 | (4S,7S,14R,16aS,20aS)-14-benzyl-4-(4-chlorobenzyl)-11-(2-(4-(2- |
| | ((dimethylamino)methyl)-1-methyl-1H-imidazol-5-yl)phenoxy)-4,6-difluorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 42 | (2S,5S,8S,13S,16R)-16-benzyl-8-(4-chlorobenzyl)-1-(4-(difluoromethyl)-2-(4-(2- |
| | ((dimethylamino)methyl)-1-methyl-1H-imidazol-5-yl)phenoxy)benzyl)-5- |
| | (methoxymethyl)-2,7,13,14-tetramethyl-1,4,7,11,14-pentaazacyclooctadecane- |
| | 3,6,10,15,18-pentaone |
| 43 | (2S,5S,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-13-(4-(dimethylamino)butyl)- |
| | 5-(methoxymethyl)-2,7,14-trimethyl-1,4,7,11,14-pentaazacyclooctadecane- |
| | 3,6,10,15,18-pentaone; |
| 44 | (2S,5S,8S,13S,16R)-16-allyl-1-(4-chloro-2-(4-(2-((dimethylamino)methyl)-1-methyl- |
| | 1H-imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 45 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-16-((5-fluoropyridin-2-yl)methyl)- |
| | 5-(methoxymethyl)-2,7,13,14-tetramethyl-1,4,7,11,14-pentaazacyclooctadecane- |
| | 3,6,10,15,18-pentaone; |
| 46 | tert-butyl 3-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10- |
| | yl]propanoate |
| 48 | (4S,7S,10S,14R,16aS,20aS)-14-benzyl-4-(4-chlorobenzyl)-11-(2-(4-(2- |
| | ((dimethylamino)methyl)-1-methyl-1H-imidazol-5-yl)phenoxy)-4-fluorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 49 | (2S,5S,8S,13S,16R)-16-benzyl-1-(2-(4-(2-((bis(methyl-d3)amino)methyl)-1-methyl- |
| | 1H-imidazol-5-yl)phenoxy)-4-chlorobenzyl)-8-(4-chlorobenzyl)-2-ethyl-5- |
| | (methoxymethyl)-7,13,14-trimethyl-1,4,7,11,14-pentaazacyclooctadecane- |
| | 3,6,10,15,18-pentaone; |
| 50 | (2S,5S,8S,16R)-16-benzyl-1-(4-chloro-2-(4-(2-((dimethylamino)methyl)-1-methyl- |
| | 1H-imidazol-5-yl)phenoxy)benzyl)-8-(4-chlorobenzyl)-5-(methoxymethyl)- |
| | 2,7,12,13,14-pentamethyl-1,4,7,11,14-pentaazacyclooctadecane-3,6,10,15,18- |
| | pentaone; |
| 51 | (2S,5S,8S,13S,16R)-16-benzyl-1-(4-chloro-2-(3,5-difluoro-4-(1-methyl-2-(pyrrolidin- |
| | 1-ylmethyl)-1H-imidazol-5-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 52 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-10-(2-(3-hydroxy-3-methylazetidin-1-yl)-2-oxoethyl)-7- |
| | (methoxymethyl)-5,16- |
| | dimethylhexadecahydrobenzo[l][1,4,7,11,14]pentaazacyclooctadecine- |
| | 2,6,9,12,15(3H)-pentaone; |
| 53 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(ethoxymethyl)-2,7,13,14,16- |
| | pentamethyl-1,4,7,11,14-pentaazacyclooctadecane-3,6,10,15,18-pentaone; |
| 54 | (2S,5S,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-2-(2-fluoroethyl)-5- |
| | (methoxymethyl)-7,13,14-trimethyl-1,4,7,11,14-pentaazacyclooctadecane- |
| | 3,6,10,15,18-pentaone; |
| 55 | (2S,5S,8S,13S,16R)-16-benzyl-1-(4-chloro-2-(4-(1,4-dimethyl-2-(pyrrolidin-1- |
| | ylmethyl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 56 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-16-(3-(difluoromethyl)benzyl)-5- |
| | (methoxymethyl)-2,7,13,14-tetramethyl-1,4,7,11,14-pentaazacyclooctadecane- |
| | 3,6,10,15,18-pentaone; |
| 57 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-16-(4-(difluoromethyl)benzyl)-5- |
| | (methoxymethyl)-2,7,13,14-tetramethyl-1,4,7,11,14-pentaazacyclooctadecane- |
| | 3,6,10,15,18-pentaone; |
| 58 | (4S,7S,10R,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 60 | (2S,5S,8S,13S,16R)-16-benzyl-1-(4-chloro-2-(4-(2-(((R)-3-fluoropyrrolidin-1- |
| | yl)methyl)-1-methyl-1H-imidazol-5-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 61 | (4S,7S,10S,14R)-14-benzyl-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,16-trimethyltetradecahydro-1H-pyrano[3,4- |
| | l][1,4,7,11,14]pentaazacyclooctadecine-2,6,9,12,15(3H)-pentaone; |
| 62 | (5S,8S,13S,16R)-8-(4-chlorobenzyl)-1-(2-(difluoromethoxy)-6-(4-(2- |
| | ((dimethylamino)methyl)-1-methyl-1H-imidazol-5-yl)phenoxy)benzyl)-5- |
| | (methoxymethyl)-2,7,13,14,16-pentamethyl-1,4,7,11,14-pentaazacyclooctadecane- |
| | 3,6,10,15,18-pentaone; |
| 63 | (2S,5S,8S,13S)-1-(4-chloro-2-(4-(1-methyl-2-(pyrrolidin-1-ylmethyl)-1H-imidazol-5- |
| | yl)phenoxy)benzyl)-8-(4-chlorobenzyl)-16-((1-(difluoromethyl)-1H-pyrazol-3- |
| | yl)methyl)-5-(methoxymethyl)-2,7,13,14-tetramethyl-1,4,7,11,14- |
| | pentaazacyclooctadecane-3,6,10,15,18-pentaone; |
| 64 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,14,16- |
| | tetramethylhexadecahydrobenzo[l][1,4,7,11,14]pentaazacyclooctadecine- |
| | 2,6,9,12,15(3H)-pentaone; |
| 65 | (2S,5S,8S,16R)-16-benzyl-1-(4-chloro-2-(4-(2-((dimethylamino)methyl)-1-methyl- |
| | 1H-imidazol-5-yl)phenoxy)benzyl)-8-(4-chlorobenzyl)-5-(methoxymethyl)- |
| | 2,7,12,12,14-pentamethyl-1,4,7,11,14-pentaazacyclooctadecane-3,6,10,15,18- |
| | pentaone; |
| 66 | (2S,5S,8S,13S)-8-(4-chlorobenzyl)-1-(2-(4-(2-((dimethylamino)methyl)-1-methyl- |
| | 1H-imidazol-5-yl)phenoxy)benzyl)-5-(hydroxymethyl)-2,7,13,14-tetramethyl- |
| | 1,4,7,11,14-pentaazacyclooctadecane-3,6,10,15,18-pentaone; |
| 67 | (2S,5S,8S,13S,16R)-1-(4-chloro-2-(4-(1-methyl-2-(pyrrolidin-1-ylmethyl)-1H- |
| | imidazol-5-yl)phenoxy)benzyl)-8-(4-chlorobenzyl)-16-(2,3-difluorobenzyl)-5- |
| | (methoxymethyl)-2,7,13,14-tetramethyl-1,4,7,11,14-pentaazacyclooctadecane- |
| | 3,6,10,15,18-pentaone; |
| 68 | (2S,5S,8S,13S,16R)-16-benzyl-1-(4-chloro-2-(4-(2-ethoxy-1-methyl-1H-imidazol-5- |
| | yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 69 | (2S,5S,8S,13S,16R)-16-benzyl-1-(4-chloro-2-(4-(2-methoxypyrimidin-5- |
| | yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 70 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2-ethyl-5- |
| | (methoxymethyl)-7,13,14-trimethyl-1,4,7,11,14-pentaazacyclooctadecane- |
| | 3,6,10,15,18-pentaone; |
| 71 | (2S,5S,8S,13R,16R)-methyl 16-benzyl-1-(4-chloro-2-(4-(2- |
| | ((dimethylamino)methyl)-1-methyl-1H-imidazol-5-yl)phenoxy)benzyl)-8-(4- |
| | chlorobenzyl)-5-(methoxymethyl)-2,7,14-trimethyl-3,6,10,15,18-pentaoxo- |
| | 1,4,7,11,14-pentaazacyclooctadecane-13-carboxylate; |
| 72 | (2S,5S,8S,13S,16R)-16-benzyl-1-(4-chloro-2-(3-fluoro-4-(1-methyl-2-(pyrrolidin-1- |
| | ylmethyl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 73 | (2S,5S,8S,13S,16R)-16-benzyl-1-(4-chloro-2-(4-(1-methyl-2-(1-(pyrrolidin-1- |
| | yl)ethyl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 74 | (2R,5S,8S,13S,16R)-16-benzyl-8-(4-chlorobenzyl)-1-(4-(difluoromethyl)-2-(4-(2- |
| | ((dimethylamino)methyl)-1-methyl-1H-imidazol-5-yl)phenoxy)benzyl)-5- |
| | (methoxymethyl)-2,7,13,14-tetramethyl-1,4,7,11,14-pentaazacyclooctadecane- |
| | 3,6,10,15,18-pentaone; |
| 75 | (2S,5S,8S,13S,16S)-1-(4-chloro-2-(4-(2-((dimethylamino)methyl)-1-methyl-1H- |
| | imidazol-5-yl)phenoxy)benzyl)-8-(4-chlorobenzyl)-16-((R)-1-hydroxybutyl)-5- |
| | (methoxymethyl)-2,7,13,14-tetramethyl-1,4,7,11,14-pentaazacyclooctadecane- |
| | 3,6,10,15,18-pentaone; |
| 76 | (2S,5S,8S,13S,16R)-1-(2-(4-(2-(azetidin-1-ylmethyl)-1-methyl-1H-imidazol-5- |
| | yl)phenoxy)-4-chlorobenzyl)-16-benzyl-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 77 | (1S,5S,8S,11S,15R,18R)-15-benzyl-12-(4-chloro-2-(4-(2-((dimethylamino)methyl)- |
| | 1-methyl-1H-imidazol-5-yl)phenoxy)benzyl)-5-(4-chlorobenzyl)-8-(methoxymethyl)- |
| | 6,11,17-trimethyl-2,6,9,12,17-pentaazabicyclo[16.1.0]nonadecane-3,7,10,13,16- |
| | pentaone; |
| 78 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2,5- |
| | bis(methoxymethyl)-7,13,14-trimethyl-1,4,7,11,14-pentaazacyclooctadecane- |
| | 3,6,10,15,18-pentaone; |
| 79 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-((difluoromethoxy)methyl)-5,10,16- |
| | trimethylhexadecahydrobenzo[l][1,4,7,11,14]pentaazacyclooctadecine- |
| | 2,6,9,12,15(3H)-pentaone; |
| 80 | (2S,5S,8S,13S,16R)-16-benzyl-8-(4-chlorobenzyl)-5-(methoxymethyl)-2,7,13,14- |
| | tetramethyl-1-(2-(4-(1-methyl-2-(pyrrolidin-1-ylmethyl)-1H-imidazol-5-yl)phenoxy)- |
| | 4-(trifluoromethyl)benzyl)-1,4,7,11,14-pentaazacyclooctadecane-3,6,10,15,18- |
| | pentaone; |
| 81 | (2S,5S,8S,13S,16R)-16-benzyl-1-(4-chloro-2-(4-(1-methyl-2-((E)-pent-1-en-1-yl)- |
| | 1H-imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 82 | 2-((2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,13,14-trimethyl-3,6,10,15,18-pentaoxo-1,4,7,11,14-pentaazacyclooctadecan-2- |
| | yl)acetic acid; |
| 83 | (2S,5S,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-13-(2-fluoroethyl)-5- |
| | (methoxymethyl)-2,7,14-trimethyl-1,4,7,11,14-pentaazacyclooctadecane- |
| | 3,6,10,15,18-pentaone; |
| 84 | (2S,5S,8S,13S,16R)-16-benzyl-1-(4-chloro-2-(4-(5-(1-(2-fluoroethyl)piperidin-4- |
| | yl)pyridin-3-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 85 | (4S,7S,10S,14R,16aS,20aS)-14-benzyl-4-(4-chlorobenzyl)-11-(2-(4-(2-((R)-1- |
| | (dimethylamino)ethyl)-1-methyl-1H-imidazol-5-yl)phenoxy)-4-ethylbenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 86 | (2S,5S,8S,13S,16R)-16-benzyl-1-(4-chloro-2-(4-(2-(methylamino)pyrimidin-5- |
| | yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 87 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2-(2-hydroxyethyl)-5- |
| | (methoxymethyl)-7,13,14-trimethyl-1,4,7,11,14-pentaazacyclooctadecane- |
| | 3,6,10,15,18-pentaone; |
| 88 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)-6-methylbenzyl)-5- |
| | (methoxymethyl)-2,7,13,14-tetramethyl-1,4,7,11,14-pentaazacyclooctadecane- |
| | 3,6,10,15,18-pentaone; |
| 89 | 2-((4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,16-trimethyl-2,6,9,12,15- |
| | pentaoxodocosahydrobenzo[l][1,4,7,11,14]pentaazacyclooctadecin-14- |
| | yl)acetonitrile |
| 90 | (2S,5S,8S,12S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2-(3- |
| | (dimethylamino)propyl)-5-(methoxymethyl)-7,12,13,14-tetramethyl-1,4,7,11,14- |
| | pentaazacyclooctadecane-3,6,10,15,18-pentaone; |
| 91 | (4S,7S,10R,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-10-(2- |
| | (dimethylamino)ethyl)-7-(methoxymethyl)-5,16-dimethyl-14-(pyridin-2- |
| | ylmethyl)hexadecahydrobenzo[l][1,4,7,11,14]pentaazacyclooctadecine- |
| | 2,6,9,12,15(3H)-pentaone; |
| 92 | (2S,5S,8S,13S,16R)-16-benzyl-1-(4-chloro-2-(4-(2-isopropoxy-1-methyl-1H- |
| | imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 93 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(3-morpholino-3- |
| | oxopropyl)hexadecahydrobenzo[l][1,4,7,11,14]pentaazacyclooctadecine- |
| | 2,6,9,12,15(3H)-pentaone; |
| 94 | (2S,5S,8S,12S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,12,13,14-tetramethyl-2-(2-morpholinoethyl)-1,4,7,11,14- |
| | pentaazacyclooctadecane-3,6,10,15,18-pentaone; |
| 95 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,13,14-trimethyl-2-(3-oxo-3-(2-oxa-6-azaspiro[3.3]heptan-6-yl)propyl)-1,4,7,11,14- |
| | pentaazacyclooctadecane-3,6,10,15,18-pentaone; |
| 96 | (4S,7S,10S,14S,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,16-trimethyl-14-(2,3,3- |
| | trifluoroallyl)hexadecahydrobenzo[l][1,4,7,11,14]pentaazacyclooctadecine- |
| | 2,6,9,12,15(3H)-pentaone; |
| 97 | (2S,5S,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-2-(2,2-difluoroethyl)-5- |
| | (methoxymethyl)-7,13,14-trimethyl-1,4,7,11,14-pentaazacyclooctadecane- |
| | 3,6,10,15,18-pentaone; |
| 98 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,13,14- |
| | tetramethyl-16-(2,3,5-trifluorobenzyl)-1,4,7,11,14-pentaazacyclooctadecane- |
| | 3,6,10,15,18-pentaone; |
| 99 | 2-(((2S,5S,8S,12S,13S,16R)-1-(4-chloro-2-(4-(2-((dimethylamino)methyl)-1-methyl- |
| | 1H-imidazol-5-yl)phenoxy)benzyl)-8-(4-chlorobenzyl)-5-(methoxymethyl)- |
| | 2,7,12,13,14-pentamethyl-3,6,10,15,18-pentaoxo-1,4,7,11,14- |
| | pentaazacyclooctadecan-16-yl)methyl)benzonitrile; |
| 100 | (4S,7S,10S,14R,16aS,20aS)-14-(bicyclo[1.1.1]pentan-1-ylmethyl)-11-(4-chloro-2- |
| | (4-(2-((dimethylamino)methyl)-1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 101 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(2- |
| | morpholinoethyl)hexadecahydrobenzo[l][1,4,7,11,14]pentaazacyclooctadecine- |
| | 2,6,9,12,15(3H)-pentaone; |
| 102 | (2S,5S,8S,12S,13S,16R)-16-benzyl-8-(4-chlorobenzyl)-1-(2-(4-(2- |
| | ((dimethylamino)methyl)-1-methyl-1H-imidazol-5-yl)phenoxy)-4-methylbenzyl)-5- |
| | (methoxymethyl)-2,7,12,13,14-pentamethyl-1,4,7,11,14-pentaazacyclooctadecane- |
| | 3,6,10,15,18-pentaone; |
| 103 | (2S,5S,8S,13S,16R)-1-(4-chloro-2-(4-(1-methyl-2-((S)-1-(pyrrolidin-1-yl)ethyl)-1H- |
| | imidazol-5-yl)phenoxy)benzyl)-8-(4-chlorobenzyl)-5-(methoxymethyl)-2,7,13,14,16- |
| | pentamethyl-1,4,7,11,14-pentaazacyclooctadecane-3,6,10,15,18-pentaone; |
| 104 | (4S,7S,10S,14R,16aS,20aS)-14-benzyl-11-(4-chloro-2-(4-(2-((R)-1-(dimethylamino)ethyl)- |
| | 1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 105 | (4S,7S,10S,14R,16aS,20aS)-11-(4-chloro-2-(4-(4-((dimethylamino)methyl)-5- |
| | methyl-1H-imidazol-1-yl)phenoxy)benzyl)-4-(4-chlorobenzyl)-7-(methoxymethyl)- |
| | 5,10,16-trimethyl-14-(pyridin-2- |
| | ylmethyl)hexadecahydrobenzo[l][1,4,7,11,14]pentaazacyclooctadecine- |
| | 2,6,9,12,15(3H)-pentaone; |
| 106 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(2-oxo-2-(2-oxa-6- |
| | azaspiro[3.3]heptan-6- |
| | yl)ethyl)hexadecahydrobenzo[l][1,4,7,11,14]pentaazacyclooctadecine- |
| | 2,6,9,12,15(3H)-pentaone; |
| 107 | (2S,5S,8S,12S,13S,16R)-16-allyl-8-(4-chlorobenzyl)-1-(2-(4-(2- |
| | ((dimethylamino)methyl)-1-methyl-1H-imidazol-5-yl)phenoxy)-4-methylbenzyl)-5- |
| | (methoxymethyl)-2,7,12,13,14-pentamethyl-1,4,7,11,14-pentaazacyclooctadecane- |
| | 3,6,10,15,18-pentaone; |
| 108 | (2S,5S,8S,13S,16R)-1-(4-chloro-2-(4-(1-methyl-2-(pyrrolidin-1-ylmethyl)-1H- |
| | imidazol-5-yl)phenoxy)benzyl)-8-(4-chlorobenzyl)-16-(4-fluorobenzyl)-5- |
| | (methoxymethyl)-2,7,13,14-tetramethyl-1,4,7,11,14-pentaazacyclooctadecane- |
| | 3,6,10,15,18-pentaone; |
| 109 | (4S,7S,10S,14R,16aS,20aS)-10-(2-aminoethyl)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16- |
| | dimethylhexadecahydrobenzo[l][1,4,7,11,14]pentaazacyclooctadecine- |
| | 2,6,9,12,15(3H)-pentaone; |
| 110 | (4S,7S,10S,14R,16aS,20aS)-14-allyl-11-(4-chloro-2-(4-(2-((dimethylamino)methyl)- |
| | 1-methyl-1H-imidazol-5-yl)phenoxy)benzyl)-4-(4-chlorobenzyl)-7-(methoxymethyl)- |
| | 5,10,16-trimethylhexadecahydrobenzo[l][1,4,7,11,14]pentaazacyclooctadecine- |
| | 2,6,9,12,15(3H)-pentaone; |
| 111 | 2-(((4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,16-dimethyl-2,6,9,12,15-pentaoxo-10-(3-(pyrrolidin-1- |
| | yl)propyl)docosahydrobenzo[l][1,4,7,11,14]pentaazacyclooctadecin-14- |
| | yl)methyl)pyridine 1-oxide; |
| 112 | (2S,5S,8S,13S,16R)-1-(2-(4-(4-(2-oxa-6-azaspiro[3.3]heptan-6-ylmethyl)-5-methyl- |
| | 1H-imidazol-1-yl)phenoxy)-4-chlorobenzyl)-16-benzyl-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 113 | 3-(2-(((2S,5S,8S,13S,16R)-16-benzyl-8-(4-chlorobenzyl)-5-(methoxymethyl)- |
| | 2,7,13,14-tetramethyl-3,6,10,15,18-pentaoxo-1,4,7,11,14- |
| | pentaazacyclooctadecan-1-yl)methyl)-5-chlorophenoxy)-N-(piperidin-4- |
| | yl)benzamide |
| 114 | (2S,5S,8S,13S,16R)-16-benzyl-1-(4-chloro-2-(4-(5,6,7,8-tetrahydro- |
| | [1,2,4]triazolo[4,3-a]pyrazin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 115 | (2S,5S,8S,13S,16R)-16-benzyl-1-(4-chloro-2-(4-(5,6,7,8-tetrahydro-1,6- |
| | naphthyridin-3-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 116 | (2S,5S,8S,13S,16R)-16-benzyl-1-(4-chloro-2-(4-(7-(2-methoxyacetyl)-5,6,7,8- |
| | tetrahydro-[1,2,4]triazolo[4,3-a]pyrazin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 117 | (2S,5S,8S,13S,16R)-16-benzyl-1-(4-chloro-2-(4-(6-(2-methoxyacetyl)-5,6,7,8- |
| | tetrahydro-1,6-naphthyridin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 118 | 3-(2-(((2S,5S,8S,13S,16R)-16-benzyl-8-(4-chlorobenzyl)-5-(methoxymethyl)- |
| | 2,7,13,14-tetramethyl-3,6,10,15,18-pentaoxo-1,4,7,11,14- |
| | pentaazacyclooctadecan-1-yl)methyl)-5-chlorophenoxy)-N-(1-methylpiperidin-4- |
| | yl)benzamide; |
| 119 | (2S,5S,8S,13S,16R)-16-benzyl-1-(4-chloro-2-(4-(5-(pyrrolidin-2-yl)pyridin-3- |
| | yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 120 | (2S,5S,8S,13S,16R)-16-benzyl-1-(4-chloro-2-(4-(4-((diethylamino)methyl)-1H- |
| | 1,2,3-triazol-1-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 121 | (2S,5S,8S,13S,16R)-16-benzyl-1-(4-chloro-2-(4-(5,6,7,8-tetrahydroimidazo[1,2- |
| | a]pyrazin-3-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 122 | 5-(4-(2-(((2S,5S,8S,13S,16R)-16-benzyl-8-(4-chlorobenzyl)-5-(methoxymethyl)- |
| | 2,7,13,14-tetramethyl-3,6,10,15,18-pentaoxo-1,4,7,11,14- |
| | pentaazacyclooctadecan-1-yl)methyl)-5-chlorophenoxy)phenyl)-N-(piperidin-4- |
| | yl)picolinamide; |
| 123 | (2S,5S,8S,13S,16R)-16-benzyl-1-(4-chloro-2-(4-(7-isopropyl-5,6,7,8- |
| | tetrahydroimidazo[1,2-a]pyrazin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 124 | (2S,5S,8S,13S,16R)-16-benzyl-1-(4-chloro-2-(4-(7-(pyrrolidine-3-carbonyl)-5,6,7,8- |
| | tetrahydroimidazo[1,2-a]pyrazin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 125 | (2S,5S,8S,13S,16R)-16-benzyl-1-(4-chloro-2-((3-oxo-2-(piperidin-4-yl)isoindolin-5- |
| | yl)oxy)benzyl)-8-(4-chlorobenzyl)-5-(hydroxymethyl)-2,7,13,14-tetramethyl- |
| | 1,4,7,11,14-pentaazacyclooctadecane-3,6,10,15,18-pentaone; |
| 126 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 127 | (2S,5S,8S,13S,16R)-16-benzyl-1-(4-chloro-2-(4-(4-(pyrrolidin-1-ylmethyl)-1H-1,2,3- |
| | triazol-1-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 128 | (2S,5S,8S,13S,16R)-16-benzyl-1-(4-chloro-2-(4-(4-(piperidin-1-ylmethyl)-1H-1,2,3- |
| | triazol-1-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 129 | (2S,5S,8S,13S,16R)-16-benzyl-1-(4-chloro-2-(4-(7-((R)-pyrrolidine-3-carbonyl)- |
| | 5,6,7,8-tetrahydro-[1,2,4]triazolo[4,3-a]pyrazin-3-yl)phenoxy)benzyl)-8-(4- |
| | chlorobenzyl)-5-(methoxymethyl)-2,7,13,14-tetramethyl-1,4,7,11,14- |
| | pentaazacyclooctadecane-3,6,10,15,18-pentaone; |
| 130 | (2S,5S,8S,13S,16R)-16-benzyl-1-(4-chloro-2-((2-(1-methylpiperidin-4-yl)-3- |
| | oxoisoindolin-5-yl)oxy)benzyl)-8-(4-chlorobenzyl)-5-(hydroxymethyl)-2,7,13,14- |
| | tetramethyl-1,4,7,11,14-pentaazacyclooctadecane-3,6,10,15,18-pentaone; |
| 131 | (2S,5S,8S,13S,16R)-16-benzyl-1-(4-chloro-2-(4-(7-((S)-pyrrolidine-3-carbonyl)- |
| | 5,6,7,8-tetrahydro-[1,2,4]triazolo[4,3-a]pyrazin-3-yl)phenoxy)benzyl)-8-(4- |
| | chlorobenzyl)-5-(methoxymethyl)-2,7,13,14-tetramethyl-1,4,7,11,14- |
| | pentaazacyclooctadecane-3,6,10,15,18-pentaone; |
| 132 | (2S,5S,8S,13S,16R)-16-benzyl-1-(4-chloro-2-(4-(7-isobutyryl-5,6,7,8- |
| | tetrahydroimidazo[1,2-a]pyrazin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 133 | (2S,5S,8S,13S,16R)-16-benzyl-1-(4-chloro-2-(4-(7-(tetrahydrofuran-3-carbonyl)- |
| | 5,6,7,8-tetrahydroimidazo[1,2-a]pyrazin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 134 | (2S,5S,8S,13S,16R)-16-benzyl-1-(4-chloro-2-(4-(4-((dimethylamino)methyl)-5- |
| | methyl-1H-1,2,3-triazol-1-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 135 | (2S,5S,8S,13S,16R)-16-benzyl-1-(4-chloro-2-(4-(5-(1,4-dimethylpiperazin-2- |
| | yl)pyridin-3-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 136 | (2S,5S,8S,13S,16R)-16-benzyl-1-(4-chloro-2-(4-(1′,2′,3′,6′-tetrahydro-[3,4′- |
| | bipyridin]-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 137 | (2S,5S,8S,13S,16R)-16-benzyl-1-(4-chloro-2-(4-(5-(piperidin-4-yl)pyridin-3- |
| | yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 138 | (2S,5S,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 139 | (2S,5S,8S,13S,16R)-16-benzyl-1-(4-chloro-2-((2-(1-(cyclopropylmethyl)piperidin-4- |
| | yl)-3-oxoisoindolin-5-yl)oxy)benzyl)-8-(4-chlorobenzyl)-5-(hydroxymethyl)- |
| | 2,7,13,14-tetramethyl-1,4,7,11,14-pentaazacyclooctadecane-3,6,10,15,18- |
| | pentaone; |
| 141 | (2S,5S,8S)-1-(4-chloro-2-(4-(2-((dimethylamino)methyl)-1-methyl-1H-imidazol-5- |
| | yl)phenoxy)benzyl)-8-(4-chlorobenzyl)-5-(methoxymethyl)-2,7,14-trimethyl- |
| | 1,4,7,11,14-pentaazacyclooctadecane-3,6,10,15,18-pentaone; |
| 142 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(hydroxymethyl)- |
| | 2,7,13,14-tetramethyl-1,4,7,11,14-pentaazacyclooctadecane-3,6,10,15,18- |
| | pentaone; |
| 143 | (2S,5S,8S,13S,16R)-16-benzyl-8-(4-chlorobenzyl)-5-(hydroxymethyl)-2,7,13,14- |
| | tetramethyl-1-(2-(2-oxo-2-(5H-pyrrolo[3,4-b]pyridin-6(7H)-yl)ethoxy)benzyl)- |
| | 1,4,7,11,14-pentaazacyclooctadecane-3,6,10,15,18-pentaone; |
| 144 | (6R,10S,13S,16S)-6-benzyl-9-(4-chloro-2-(4-(2-((dimethylamino)methyl)-1-methyl- |
| | 1H-imidazol-5-yl)phenoxy)benzyl)-16-(4-chlorobenzyl)-13-(methoxymethyl)- |
| | 4,10,15-trimethyl-4,9,12,15,19-pentaazaspiro[2.17]icosane-5,8,11,14,18-pentaone; |
| 145 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 146 | (2S,5S,8S,13S,16R)-16-benzyl-1-(4-chloro-2-(4-(4-methyl-5-(pyrrolidin-1- |
| | ylmethyl)pyridin-3-yl)phenoxy)benzyl)-8-(4-chlorobenzyl)-5-(hydroxymethyl)- |
| | 2,7,13,14-tetramethyl-1,4,7,11,14-pentaazacyclooctadecane-3,6,10,15,18- |
| | pentaone; |
| 147 | (1R,5S,8S,11S,15R,18S)-15-benzyl-12-(4-chloro-2-(4-(2-((dimethylamino)methyl)- |
| | 1-methyl-1H-imidazol-5-yl)phenoxy)benzyl)-5-(4-chlorobenzyl)-8-(methoxymethyl)- |
| | 6,11,17-trimethyl-2,6,9,12,17-pentaazabicyclo[16.1.0]nonadecane-3,7,10,13,16- |
| | pentaone; |
| 148 | (2S,5S,8S,13S,16R)-5-((1H-tetrazol-5-yl)methyl)-16-benzyl-1-(4-chloro-2-(4-(2- |
| | ((dimethylamino)methyl)-1-methyl-1H-imidazol-5-yl)phenoxy)benzyl)-8-(4- |
| | chlorobenzyl)-2,7,13,14-tetramethyl-1,4,7,11,14-pentaazacyclooctadecane- |
| | 3,6,10,15,18-pentaone; |
| 149 | ethyl 1-(4-(2-(((2S,5S,8S,13S,16R)-16-benzyl-8-(4-chlorobenzyl)-5- |
| | (methoxymethyl)-2,7,13,14-tetramethyl-3,6,10,15,18-pentaoxo-1,4,7,11,14- |
| | pentaazacyclooctadecan-1-yl)methyl)-5-chlorophenoxy)phenyl)-5-methyl-1H- |
| | imidazole-4-carboxylate; |
| 150 | (2S,5S,8S,13S,16R)-16-benzyl-8-(4-chlorobenzyl)-1-(4-ethyl-2-(4-(1-methyl-2- |
| | (pyrrolidin-1-ylmethyl)-1H-imidazol-5-yl)phenoxy)benzyl)-5-(methoxymethyl)- |
| | 2,7,13,14-tetramethyl-1,4,7,11,14-pentaazacyclooctadecane-3,6,10,15,18- |
| | pentaone; |
| 151 | (2S,5S,8S,13S,16R)-16-benzyl-1-(4-chloro-2-(4-(2-(1-hydroxypropyl)-1-methyl-1H- |
| | imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 152 | (4S,7S,10S,14R,16aS,20aS)-14-benzyl-11-(4-chloro-2-(4-(1-methyl-2-((R)-1- |
| | (pyrrolidin-1-yl)ethyl)-1H-imidazol-5-yl)phenoxy)benzyl)-4-(4-chlorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 154 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)-6-methoxybenzyl)-5- |
| | (methoxymethyl)-2,7,13,14-tetramethyl-1,4,7,11,14-pentaazacyclooctadecane- |
| | 3,6,10,15,18-pentaone; |
| 155 | (2S,5S,8S,13S,16R)-1-(4-chloro-2-(4-(1-methyl-2-(pyrrolidin-1-ylmethyl)-1H- |
| | imidazol-5-yl)phenoxy)benzyl)-8-(4-chlorobenzyl)-16-(3-fluorobenzyl)-5- |
| | (methoxymethyl)-2,7,13,14-tetramethyl-1,4,7,11,14-pentaazacyclooctadecane- |
| | 3,6,10,15,18-pentaone; |
| 156 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2-(2-methoxyethyl)-5- |
| | (methoxymethyl)-7,13,14-trimethyl-1,4,7,11,14-pentaazacyclooctadecane- |
| | 3,6,10,15,18-pentaone; |
| 157 | (4S,7S,10S,14R,16aS,20aS)-11-(2-(4-(2-(2-oxa-6-azaspiro[3.3]heptan-6-ylmethyl)- |
| | 1-methyl-1H-imidazol-5-yl)phenoxy)-4-chlorobenzyl)-14-benzyl-4-(4-chlorobenzyl)- |
| | 7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 158 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,13,14-trimethyl-2-(3-morpholinopropyl)-1,4,7,11,14-pentaazacyclooctadecane- |
| | 3,6,10,15,18-pentaone; |
| 159 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-16-methoxy-5-(methoxymethyl)- |
| | 2,7,13,14-tetramethyl-1,4,7,11,14-pentaazacyclooctadecane-3,6,10,15,18- |
| | pentaone; |
| 160 | 2-((2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2,7,13,14-tetramethyl- |
| | 3,6,10,15,18-pentaoxo-1,4,7,11,14-pentaazacyclooctadecan-5-yl)-N,N- |
| | dimethylacetamide; |
| 161 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,13,14,16- |
| | pentamethyl-1,4,7,11,14-pentaazacyclooctadecane-3,6,10,15,18-pentaone; |
| 162 | (2S,5S,8S,13S,16R)-16-benzyl-1-(4-chloro-2-(4-(7-((S)-tetrahydrofuran-3- |
| | carbonyl)-5,6,7,8-tetrahydroimidazo[1,2-a]pyrazin-3-yl)phenoxy)benzyl)-8-(4- |
| | chlorobenzyl)-5-(methoxymethyl)-2,7,13,14-tetramethyl-1,4,7,11,14- |
| | pentaazacyclooctadecane-3,6,10,15,18-pentaone; |
| 163 | 1-(4-(2-(((2S,5S,8S,13S,16R)-16-benzyl-8-(4-chlorobenzyl)-5-(methoxymethyl)- |
| | 2,7,13,14-tetramethyl-3,6,10,15,18-pentaoxo-1,4,7,11,14- |
| | pentaazacyclooctadecan-1-yl)methyl)-5-chlorophenoxy)phenyl)-N-isopropyl-5- |
| | methyl-1H-imidazole-4-carboxamide; |
| 164 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(hydroxymethyl)- |
| | 5,10,14,16- |
| | tetramethylhexadecahydrobenzo[l][1,4,7,11,14]pentaazacyclooctadecine- |
| | 2,6,9,12,15(3H)-pentaone; |
| 165 | (2S,5S,8S,13S,16R)-1-(4-chloro-2-(4-(1-methyl-2-(pyrrolidin-1-ylmethyl)-1H- |
| | imidazol-5-yl)phenoxy)benzyl)-8-(4-chlorobenzyl)-16-(3,4-difluorobenzyl)-5- |
| | (methoxymethyl)-2,7,13,14-tetramethyl-1,4,7,11,14-pentaazacyclooctadecane- |
| | 3,6,10,15,18-pentaone; |
| 166 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)-3,6-difluorobenzyl)-5- |
| | (methoxymethyl)-2,7,13,14-tetramethyl-1,4,7,11,14-pentaazacyclooctadecane- |
| | 3,6,10,15,18-pentaone; |
| 167 | (2S,5S,8S,13S,16R)-16-benzyl-1-(4-chloro-2-(4-(2-(1-hydroxyethyl)-1-methyl-1H- |
| | imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 168 | (2S,5S,8S,13S,16R)-16-benzyl-8-(4-chlorobenzyl)-1-((4-(4-(2- |
| | ((dimethylamino)methyl)-1-methyl-1H-imidazol-5-yl)phenoxy)-6-oxo-1,6- |
| | dihydropyridin-3-yl)methyl)-5-(methoxymethyl)-2,7,13,14-tetramethyl-1,4,7,11,14- |
| | pentaazacyclooctadecane-3,6,10,15,18-pentaone; |
| 169 | (2S,5S,8S,13S,16R)-13-(4-aminobutyl)-16-benzyl-1-(4-chloro-2-(4-(1-methyl-2- |
| | (pyrrolidin-1-ylmethyl)-1H-imidazol-5-yl)phenoxy)benzyl)-8-(4-chlorobenzyl)-5- |
| | (methoxymethyl)-2,7,14-trimethyl-1,4,7,11,14-pentaazacyclooctadecane- |
| | 3,6,10,15,18-pentaone; |
| 170 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5- |
| | ((difluoromethoxy)methyl)-2,7,13,14-tetramethyl-1,4,7,11,14- |
| | pentaazacyclooctadecane-3,6,10,15,18-pentaone; |
| 171 | (2S,5S,8S,13S,16R)-16-benzyl-1-(4-chloro-2-(4-(7-((R)-tetrahydrofuran-3- |
| | carbonyl)-5,6,7,8-tetrahydroimidazo[1,2-a]pyrazin-3-yl)phenoxy)benzyl)-8-(4- |
| | chlorobenzyl)-5-(methoxymethyl)-2,7,13,14-tetramethyl-1,4,7,11,14- |
| | pentaazacyclooctadecane-3,6,10,15,18-pentaone; |
| 172 | 2-(((4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,16-trimethyl-2,6,9,12,15- |
| | pentaoxodocosahydrobenzo[l][1,4,7,11,14]pentaazacyclooctadecin-14- |
| | yl)methyl)pyridine 1-oxide; |
| 173 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-2-ethyl-5-(methoxymethyl)- |
| | 7,13,14,16-tetramethyl-1,4,7,11,14-pentaazacyclooctadecane-3,6,10,15,18- |
| | pentaone; |
| 174 | tert-butyl 2-((2S,5S,8S,13S,16R)-16-benzyl-1-(4-chloro-2-(4-(2- |
| | ((dimethylamino)methyl)-1-methyl-1H-imidazol-5-yl)phenoxy)benzyl)-8-(4- |
| | chlorobenzyl)-5-(methoxymethyl)-7,13,14-trimethyl-3,6,10,15,18-pentaoxo- |
| | 1,4,7,11,14-pentaazacyclooctadecan-2-yl)acetate; |
| 175 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,13,14-trimethyl-2-(3-morpholino-3-oxopropyl)-1,4,7,11,14- |
| | pentaazacyclooctadecane-3,6,10,15,18-pentaone; |
| 176 | (2S,5S,8S,13S,16R)-16-benzyl-1-(4-chloro-2-(4-(6-((S)-pyrrolidine-3-carbonyl)- |
| | 5,6,7,8-tetrahydro-1,6-naphthyridin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 177 | (2S,5S,8S,13R,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 2,7,14-trimethyl-3,6,10,15,18-pentaoxo-1,4,7,11,14-pentaazacyclooctadecane-13- |
| | carboxylic acid; |
| 178 | (2S,5S,8S,13S,16R)-16-benzyl-1-(4-chloro-2-(4-(1-methyl-2-(1-(pyrrolidin-1- |
| | yl)ethyl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 179 | (2S,5S,8S,13S,16R)-16-benzyl-8-(4-chlorobenzyl)-1-(4-(difluoromethoxy)-2-(4-(2- |
| | ((dimethylamino)methyl)-1-methyl-1H-imidazol-5-yl)phenoxy)benzyl)-5- |
| | (methoxymethyl)-2,7,13,14-tetramethyl-1,4,7,11,14-pentaazacyclooctadecane- |
| | 3,6,10,15,18-pentaone; |
| 180 | 2-(((2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,13,14- |
| | tetramethyl-3,6,10,15,18-pentaoxo-1,4,7,11,14-pentaazacyclooctadecan-16- |
| | yl)methyl)-5-fluoropyridine 1-oxide |
| 181 | (2S,5S,8S,13S,16R)-16-benzyl-1-(4-chloro-2-(4-(2-ethyl-1-methyl-1H-imidazol-5- |
| | yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 182 | tert-butyl (3-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10- |
| | yl)propyl)carbamate; |
| 183 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-16-(cyclohexylmethyl)-5- |
| | (methoxymethyl)-2,7,13,14-tetramethyl-1,4,7,11,14-pentaazacyclooctadecane- |
| | 3,6,10,15,18-pentaone; |
| 184 | (5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-13-isobutyl-5-(methoxymethyl)- |
| | 2,7,14,16-tetramethyl-1,4,7,11,14-pentaazacyclooctadecane-3,6,10,15,18- |
| | pentaone; |
| 185 | tert-butyl 2-((2S,5S,8S,13S,16R)-16-benzyl-1-(4-chloro-2-(4-(2- |
| | ((dimethylamino)methyl)-1-methyl-1H-imidazol-5-yl)phenoxy)benzyl)-8-(4- |
| | chlorobenzyl)-2,7,13,14-tetramethyl-3,6,10,15,18-pentaoxo-1,4,7,11,14- |
| | pentaazacyclooctadecan-5-yl)acetate; |
| 186 | (2S,5S,8S,12S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 2,7,12,13,14-pentamethyl-1,4,7,11,14-pentaazacyclooctadecane-3,6,10,15,18- |
| | pentaone; |
| 187 | (4S,7S,10S,14R,16aS,20aR)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,10,16-trimethyltetradecahydro-1H-pyrano[3,4- |
| | l][1,4,7,11,14]pentaazacyclooctadecine-2,6,9,12,15(3H)-pentaone; |
| 188 | 1-(4-(2-(((2S,5S,8S,13S,16R)-16-benzyl-8-(4-chlorobenzyl)-5-(methoxymethyl)- |
| | 2,7,13,14-tetramethyl-3,6,10,15,18-pentaoxo-1,4,7,11,14- |
| | pentaazacyclooctadecan-1-yl)methyl)-5-chlorophenoxy)phenyl)-N-(2-hydroxyethyl)- |
| | 5-methyl-1H-imidazole-4-carboxamide; |
| 189 | (2S,5S,8S,13S,16R)-1-(2-(4-(2-(2-oxa-6-azaspiro[3.3]heptan-6-ylmethyl)-1-methyl- |
| | 1H-imidazol-5-yl)phenoxy)-4-chlorobenzyl)-16-benzyl-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 190 | (2S,5S,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-13-(2-(dimethylamino)ethyl)- |
| | 5-(methoxymethyl)-2,7,14-trimethyl-1,4,7,11,14-pentaazacyclooctadecane- |
| | 3,6,10,15,18-pentaone; |
| 191 | tert-butyl (3-((4S,7S,10S,14R,16aS,20aS)-14-benzyl-11-(4-chloro-2-(4-(1-methyl-2- |
| | (pyrrolidin-1-ylmethyl)-1H-imidazol-5-yl)phenoxy)benzyl)-4-(4-chlorobenzyl)-7- |
| | (methoxymethyl)-5,16-dimethyl-2,6,9,12,15- |
| | pentaoxodocosahydrobenzo[l][1,4,7,11,14]pentaazacyclooctadecin-10- |
| | yl)propyl)carbamate; |
| 192 | (2S,5S,8S,13S,16R)-2-(3-aminopropyl)-16-benzyl-1-(4-chloro-2-(4-(2- |
| | ((dimethylamino)methyl)-1-methyl-1H-imidazol-5-yl)phenoxy)benzyl)-8-(4- |
| | chlorobenzyl)-5-(methoxymethyl)-7,13,14-trimethyl-1,4,7,11,14- |
| | pentaazacyclooctadecane-3,6,10,15,18-pentaone; |
| 193 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(hydroxymethyl)-2,7,13,14,16- |
| | pentamethyl-1,4,7,11,14-pentaazacyclooctadecane-3,6,10,15,18-pentaone; |
| 194 | (4S,7S,10R,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(hydroxymethyl)-5,10,16- |
| | trimethylhexadecahydrobenzo[l][1,4,7,11,14]pentaazacyclooctadecine- |
| | 2,6,9,12,15(3H)-pentaone; |
| 195 | (2S,5S,8S,13S)-1-(4-chloro-2-(4-(2-((dimethylamino)methyl)-1-methyl-1H-imidazol- |
| | 5-yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 196 | (4S,7S,10S,16aS,20aS)-4-(4-chlorobenzyl)-11-(2-(difluoromethoxy)-6-(4-(2- |
| | ((dimethylamino)methyl)-1-methyl-1H-imidazol-5-yl)phenoxy)benzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 197 | (2S,5S,8S,13S,16R)-16-benzyl-1-(4-chloro-2-(4-(2-methoxy-1-methyl-1H-imidazol- |
| | 5-yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 198 | (2S,5S,8S,12S,13S,16R)-16-benzyl-1-(4-chloro-2-(4-(1-methyl-2-(pyrrolidin-1- |
| | ylmethyl)-1H-imidazol-5-yl)phenoxy)benzyl)-8-(4-chlorobenzyl)-5-(methoxymethyl)- |
| | 2,7,12,13,14-pentamethyl-1,4,7,11,14-pentaazacyclooctadecane-3,6,10,15,18- |
| | pentaone; |
| 199 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)-4-fluorobenzyl)-5- |
| | (methoxymethyl)-2,7,13,14-tetramethyl-1,4,7,11,14-pentaazacyclooctadecane- |
| | 3,6,10,15,18-pentaone; |
| 200 | (2S,5S,8S,13S)-8-(4-chlorobenzyl)-1-(2-(4-(2-((dimethylamino)methyl)-1-methyl- |
| | 1H-imidazol-5-yl)phenoxy)benzyl)-5-(hydroxymethyl)-2-(methoxymethyl)-7,13,14- |
| | trimethyl-1,4,7,11,14-pentaazacyclooctadecane-3,6,10,15,18-pentaone; |
| 201 | (6S,11S,14S,17S)-18-(4-chloro-2-(4-(2-((dimethylamino)methyl)-1-methyl-1H- |
| | imidazol-5-yl)phenoxy)benzyl)-11-(4-chlorobenzyl)-14-(methoxymethyl)-5,6,12,17- |
| | tetramethyl-5,8,12,15,18-pentaazaspiro[2.17]icosane-4,9,13,16,19-pentaone; |
| 202 | (4S,7S,10S,16aS,20aS)-11-(4-chloro-2-(4-(2-((dimethylamino)methyl)-1-methyl- |
| | 1H-imidazol-5-yl)phenoxy)benzyl)-4-(4-chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 203 | (4S,7S,10S,14R,16aS,20aS)-14-benzyl-11-(4-chloro-2-(4-(1-methyl-2-(pyrrolidin-1- |
| | ylmethyl)-1H-imidazol-5-yl)phenoxy)benzyl)-4-(4-chlorobenzyl)-7-(methoxymethyl)- |
| | 5,10,16-trimethylhexadecahydrobenzo[l][1,4,7,11,14]pentaazacyclooctadecine- |
| | 2,6,9,12,15(3H)-pentaone; |
| 204 | (4S,7S,10R,14R,16aS,20aS)-10-(2-aminoethyl)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16- |
| | dimethylhexadecahydrobenzo[l][1,4,7,11,14]pentaazacyclooctadecine- |
| | 2,6,9,12,15(3H)-pentaone; |
| 205 | (2S,5S,8S,13S,16R)-16-benzyl-1-(4-chloro-2-(4-(1-methyl-2-(morpholinomethyl)- |
| | 1H-imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 206 | (2S,5S,8S,13S,16R)-1-(4-chloro-2-(4-(1-methyl-2-(pyrrolidin-1-ylmethyl)-1H- |
| | imidazol-5-yl)phenoxy)benzyl)-8-(4-chlorobenzyl)-5-(methoxymethyl)-2,7,13,14- |
| | tetramethyl-16-(3-(trifluoromethoxy)benzyl)-1,4,7,11,14-pentaazacyclooctadecane- |
| | 3,6,10,15,18-pentaone; |
| 207 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2-ethyl-5- |
| | (hydroxymethyl)-7,13,14-trimethyl-1,4,7,11,14-pentaazacyclooctadecane- |
| | 3,6,10,15,18-pentaone; |
| 208 | (4S,7S,10S,14R,16aS,20aS)-14-benzyl-4-(4-chlorobenzyl)-11-(2-(4-(2- |
| | ((dimethylamino)methyl)-1-methyl-1H-imidazol-5-yl)phenoxy)-4,6-difluorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 209 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chloro-3-fluorobenzyl)-2-ethyl-5- |
| | (methoxymethyl)-7,13,14-trimethyl-1,4,7,11,14-pentaazacyclooctadecane- |
| | 3,6,10,15,18-pentaone; |
| 210 | (2S,5S,8S,13S,16R)-16-benzyl-8-(4-chlorobenzyl)-1-((2-(4-(2- |
| | ((dimethylamino)methyl)-1-methyl-1H-imidazol-5-yl)phenoxy)pyridin-3-yl)methyl)-5- |
| | (methoxymethyl)-2,7,13,14-tetramethyl-1,4,7,11,14-pentaazacyclooctadecane- |
| | 3,6,10,15,18-pentaone; |
| 211 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,13,14-trimethyl-2-(2-(methylsulfonyl)ethyl)-1,4,7,11,14- |
| | pentaazacyclooctadecane-3,6,10,15,18-pentaone; |
| 212 | (2S,5S,8S,13S,16R)-16-benzyl-1-(4-chloro-2-(4-(6-((R)-pyrrolidine-3-carbonyl)- |
| | 5,6,7,8-tetrahydro-1,6-naphthyridin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 213 | 2-((2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2,7,13,14-tetramethyl- |
| | 3,6,10,15,18-pentaoxo-1,4,7,11,14-pentaazacyclooctadecan-5-yl)acetic acid |
| 214 | (2S,5S,8S,13S,16R)-16-benzyl-8-(4-chlorobenzyl)-1-((3-(4-(2- |
| | ((dimethylamino)methyl)-1-methyl-1H-imidazol-5-yl)phenoxy)pyridin-2-yl)methyl)-5- |
| | (methoxymethyl)-2,7,13,14-tetramethyl-1,4,7,11,14-pentaazacyclooctadecane- |
| | 3,6,10,15,18-pentaone; |
| 215 | (2S,5S,8S,13S)-1-(4-chloro-2-(4-(2-((dimethylamino)methyl)-1-methyl-1H-imidazol- |
| | 5-yl)phenoxy)benzyl)-8-(4-chlorobenzyl)-16-cyclopropyl-5-(methoxymethyl)- |
| | 2,7,13,14-tetramethyl-1,4,7,11,14-pentaazacyclooctadecane-3,6,10,15,18- |
| | pentaone; |
| 216 | (2S,5S,8S,13S,16R)-16-benzyl-1-(4-chloro-2-(4-(1-methyl-2-(1-(pyrrolidin-1- |
| | yl)ethyl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 217 | N-(4-((2S,5S,8S,13S,16R)-16-benzyl-1-(4-chloro-2-(4-(1-methyl-2-(pyrrolidin-1- |
| | ylmethyl)-1H-imidazol-5-yl)phenoxy)benzyl)-8-(4-chlorobenzyl)-5-(methoxymethyl)- |
| | 2,7,14-trimethyl-3,6,10,15,18-pentaoxo-1,4,7,11,14-pentaazacyclooctadecan-13- |
| | yl)butyl)acetamide |
| 218 | 2-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10-yl)-N ,N- |
| | bis(2-methoxyethyl)acetamide |
| 219 | 2-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10-yl)acetic |
| | acid |
| 220 | (2S,5S,8S,13S,16R)-16-benzyl-1-(4-chloro-2-(4-(2-(((S)-3-hydroxypyrrolidin-1- |
| | yl)methyl)-1-methyl-1H-imidazol-5-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 221 | 2-((2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2,7,13,14-tetramethyl- |
| | 3,6,10,15,18-pentaoxo-1,4,7,11,14-pentaazacyclooctadecan-5-yl)-N- |
| | methylacetamide |
| 222 | (2R,5S,8S,13S,16R)-16-benzyl-1-(4-chloro-2-(4-(2-(hydroxymethyl)-1-methyl-1H- |
| | imidazol-5-yl)phenoxy)benzyl)-8-(4-chlorobenzyl)-2,5-bis(methoxymethyl)-7,13,14- |
| | trimethyl-1,4,7,11,14-pentaazacyclooctadecane-3,6,10,15,18-pentaone; |
| 223 | (4S,7S,10S,14R,16aS,20aS)-14-benzyl-4-(4-chlorobenzyl)-11-(4-ethyl-2-(4-(1- |
| | methyl-2-(pyrrolidin-1-ylmethyl)-1H-imidazol-5-yl)phenoxy)benzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 224 | (4S,7S,10S,14R,16aR,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,10,16-trimethyltetradecahydro-1H-pyrano[3,4- |
| | l][1,4,7,11,14]pentaazacyclooctadecine-2,6,9,12,15(3H)-pentaone; |
| 225 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2-(4- |
| | (dimethylamino)butyl)-5-(methoxymethyl)-7,13,14-trimethyl-1,4,7,11,14- |
| | pentaazacyclooctadecane-3,6,10,15,18-pentaone; |
| 226 | ((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-5,10,16-trimethyl-2,6,9,12,15- |
| | pentaoxodocosahydrobenzo[l][1,4,7,11,14]pentaazacyclooctadecin-7-yl)methyl |
| | pivalate; |
| 227 | (5S,8S,11S,16S)-4-(4-chloro-2-(4-(2-((dimethylamino)methyl)-1-methyl-1H- |
| | imidazol-5-yl)phenoxy)benzyl)-11-(4-chlorobenzyl)-8-(methoxymethyl)-5,10,17- |
| | trimethyl-1,4,7,10,14,17-hexaazabicyclo[14.2.1]nonadecane-3,6,9,13,18-pentaone; |
| 228 | (4S,7S,14R,16aS,20aS)-14-benzyl-4-(4-chlorobenzyl)-11-(2-(4-(2- |
| | ((dimethylamino)methyl)-1-methyl-1H-imidazol-5-yl)phenoxy)-4-fluorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 229 | (2S,5S,8S,13S,16R)-1-(4-chloro-2-(4-(1-methyl-2-(pyrrolidin-1-ylmethyl)-1H- |
| | imidazol-5-yl)phenoxy)benzyl)-8-(4-chlorobenzyl)-16-(2-fluorobenzyl)-5- |
| | (methoxymethyl)-2,7,13,14-tetramethyl-1,4,7,11,14-pentaazacyclooctadecane- |
| | 3,6,10,15,18-pentaone; |
| 230 | (2S,5S,8S,13S,16R)-1-(2-chloro-6-(4-(2-((dimethylamino)methyl)-1-methyl-1H- |
| | imidazol-5-yl)phenoxy)benzyl)-8-(4-chlorobenzyl)-5-(methoxymethyl)-2,7,13,14,16- |
| | pentamethyl-1,4,7,11,14-pentaazacyclooctadecane-3,6,10,15,18-pentaone; |
| 232 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)-3-fluorobenzyl)-5- |
| | (methoxymethyl)-2,7,13,14-tetramethyl-1,4,7,11,14-pentaazacyclooctadecane- |
| | 3,6,10,15,18-pentaone; |
| 233 | 3-((2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-2,7,13,14-tetramethyl- |
| | 3,6,10,15,18-pentaoxo-1,4,7,11,14-pentaazacyclooctadecan-5-yl)propanoic acid; |
| 234 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7,10-bis(methoxymethyl)-5,16- |
| | dimethylhexadecahydrobenzo[l][1,4,7,11,14]pentaazacyclooctadecine- |
| | 2,6,9,12,15(3H)-pentaone; |
| 235 | (2S,5S,8S,13R,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-13-(hydroxymethyl)-5- |
| | (methoxymethyl)-2,7,14-trimethyl-1,4,7,11,14-pentaazacyclooctadecane- |
| | 3,6,10,15,18-pentaone; |
| 236 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)-4,6-difluorobenzyl)-5- |
| | (methoxymethyl)-2,7,13,14-tetramethyl-1,4,7,11,14-pentaazacyclooctadecane- |
| | 3,6,10,15,18-pentaone; |
| 237 | tert-butyl 2-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10-yl)acetate; |
| 238 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-16-(3-(difluoromethoxy)benzyl)- |
| | 5-(methoxymethyl)-2,7,13,14-tetramethyl-1,4,7,11,14-pentaazacyclooctadecane- |
| | 3,6,10,15,18-pentaone; |
| 239 | 2-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10-yl)-N-(2- |
| | methoxyethyl)-N-methylacetamide; |
| 240 | (2S,5S,8S,13S,16R)-16-benzyl-8-(4-chlorobenzyl)-1-(2-(4-(2- |
| | ((dimethylamino)methyl)-1-methyl-1H-imidazol-5-yl)phenoxy)-4-methylbenzyl)-5- |
| | (methoxymethyl)-2,7,13,14-tetramethyl-1,4,7,11,14-pentaazacyclooctadecane- |
| | 3,6,10,15,18-pentaone; |
| 241 | (4S,7S,10S,14R,16aS,20aS)-14-benzyl-11-(4-chloro-2-(4-(2-((S)-1- |
| | (dimethylamino)ethyl)-1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 242 | (4S,7S,10S,14R,16aS,20aS)-14-benzyl-11-(4-chloro-2-(4-(2-(1-hydroxyethyl)-1- |
| | methyl-1H-imidazol-5-yl)phenoxy)benzyl)-4-(4-chlorobenzyl)-7-(methoxymethyl)- |
| | 5,10,16-trimethylhexadecahydrobenzo[l][1,4,7,11,14]pentaazacyclooctadecine- |
| | 2,6,9,12,15(3H)-pentaone; |
| 243 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(hydroxymethyl)-5,10,16- |
| | trimethylhexadecahydrobenzo[l][1,4,7,11,14]pentaazacyclooctadecine- |
| | 2,6,9,12,15(3H)-pentaone; |
| 244 | 1-(4-(2-(((2S,5S,8S,13S,16R)-16-benzyl-8-(4-chlorobenzyl)-5-(methoxymethyl)- |
| | 2,7,13,14-tetramethyl-3,6,10,15,18-pentaoxo-1,4,7,11,14- |
| | pentaazacyclooctadecan-1-yl)methyl)-5-chlorophenoxy)phenyl)-N-ethyl-5-methyl- |
| | 1H-imidazole-4-carboxamide; |
| 245 | 3-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10- |
| | yl)propanoic acid; |
| 246 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,13,14- |
| | tetramethyl-16-(2,3,5,6-tetrafluorobenzyl)-1,4,7,11,14-pentaazacyclooctadecane- |
| | 3,6,10,15,18-pentaone; |
| 247 | (1R,5S,8S,11S,15R,18S)-15-benzyl-12-(4-chloro-2-(4-(2-((dimethylamino)methyl)- |
| | 1-methyl-1H-imidazol-5-yl)phenoxy)benzyl)-5-(4-chlorobenzyl)-8-(methoxymethyl)- |
| | 6,11,17-trimethyI-2,6,9,12,17-pentaazabicyclo[16.1.0]nonadecane-3,7,10,13,16- |
| | pentaone; |
| 248 | (2S,5S,8S,13S,16R)-16-benzyl-1-(4-chloro-2-(4-(2-((dimethylamino)methyl)-1- |
| | methyl-1H-imidazol-5-yl)phenoxy)benzyl)-8-(4-chloro-2-fluorobenzyl)-2-ethyl-5- |
| | (methoxymethyl)-7,13,14-trimethyl-1,4,7,11,14-pentaazacyclooctadecane- |
| | 3,6,10,15,18-pentaone; |
| 249 | 3-(4-(2-(((2S,5S,8S,13S,16R)-16-benzyl-8-(4-chlorobenzyl)-5-(methoxymethyl)- |
| | 2,7,13,14-tetramethyl-3,6,10,15,18-pentaoxo-1,4,7,11,14- |
| | pentaazacyclooctadecan-1-yl)methyl)-5-chlorophenoxy)phenyl)-6,8-dihydro-5H- |
| | spiro[imidazo[1,2-a]pyrazine-7,1′-pyrrolidin]-1′-ium chloride; |
| 250 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-10-(2- |
| | (dimethylamino)ethyl)-7-(methoxymethyl)-5,16-dimethyl-14-(pyridin-2- |
| | ylmethyl)hexadecahydrobenzo[l][1,4,7,11,14]pentaazacyclooctadecine- |
| | 2,6,9,12,15(3H)-pentaone; |
| 251 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(3-(pyrrolidin-1- |
| | yl)propyl)hexadecahydrobenzo[l][1,4,7,11,14]pentaazacyclooctadecine- |
| | 2,6,9,12,15(3H)-pentaone; |
| 252 | (4S,7S,10S,14R,16aS,20aS)-14-benzyl-11-(4-chloro-2-(4-(1-methyl-2-(pyrrolidin-1- |
| | ylmethyl)-1H-imidazol-5-yl)phenoxy)benzyl)-4-(4-chlorobenzyl)-7-(methoxymethyl)- |
| | 5,16-dimethyl-10-(3- |
| | morpholinopropyl)hexadecahydrobenzo[l][1,4,7,11,14]pentaazacyclooctadecine- |
| | 2,6,9,12,15(3H)-pentaone; |
| 253 | (4S,7S,10S,16aS,20aS)-14-((1H-pyrazol-1-yl)methyl)-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 254 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(3-(3,3,4,4-tetrafluoropyrrolidin- |
| | 1-yl)propyl)hexadecahydrobenzo[l][1,4,7,11,14]pentaazacyclooctadecine- |
| | 2,6,9,12,15(3H)-pentaone; |
| 255 | (5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-13-isobutyl-5-(methoxymethyl)- |
| | 2,7,14,16-tetramethyl-1,4,7,11,14-pentaazacyclooctadecane-3,6,10,15,18- |
| | pentaone; |
| 256 | (2S,5S,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-5-(methoxymethyl)-2,13,14- |
| | trimethyl-1,4,7,11,14-pentaazacyclooctadecane-3,6,10,15,18-pentaone; |
| 257 | (2S,5S,8S,13S,16R)-16-benzyl-1-(4-chloro-2-(4-(1-methyl-2-(pyrrolidin-1-ylmethyl)- |
| | 1H-imidazol-5-yl)phenoxy)benzyl)-8-(4-chlorobenzyl)-5-(2-fluoroethyl)-2,7,13,14- |
| | tetramethyl-1,4,7,11,14-pentaazacyclooctadecane-3,6,10,15,18-pentaone; |
| 258 | (2S,5S,8S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,13,14- |
| | tetramethyl-16-(2,3,6-trifluorobenzyl)-1,4,7,11,14-pentaazacyclooctadecane- |
| | 3,6,10,15,18-pentaone; |
| 259 | (2S,5S,8S,13S,16R)-16-benzyl-1-(4-chloro-2-(4-(5-methyl-4-(pyrrolidin-1-ylmethyl)- |
| | 1H-imidazol-1-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 260 | (4S,7S,10S,14R,16aS,20aS)-14-benzyl-11-(4-chloro-2-(4-(5-methyl-4-(pyrrolidin-1- |
| | ylmethyl)-1H-imidazol-1-yl)phenoxy)benzyl)-4-(4-chlorobenzyl)-7-(hydroxymethyl)- |
| | 5,10,16-trimethylhexadecahydrobenzo[l][1,4,7,11,14]pentaazacyclooctadecine- |
| | 2,6,9,12,15(3H)-pentaone; |
| 261 | (2S,5S,8S,12S,13S,16R)-16-benzyl-1-(4-chloro-2-(4-(2-((R)-1- |
| | (dimethylamino)ethyl)-1-methyl-1H-imidazol-5-yl)phenoxy)benzyl)-8-(4- |
| | chlorobenzyl)-5-(methoxymethyl)-2,7,12,13,14-pentamethyl-1,4,7,11,14- |
| | pentaazacyclooctadecane-3,6,10,15,18-pentaone; |
| 262 | (1S,6S,9S,12S,16S)-13-(4-chloro-2-(4-(2-((dimethylamino)methyl)-1-methyl-1H- |
| | imidazol-5-yl)phenoxy)benzyl)-6-(4-chlorobenzyl)-9-(methoxymethyl)-7,12,17,19- |
| | tetramethyl-3,7,10,13,17,19-hexaazabicyclo[14.2.2]icosane-4,8,11,14,18,20- |
| | hexaone; |
| 263 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,16-trimethyl-14- |
| | propylhexadecahydrobenzo[l][1,4,7,11,14]pentaazacyclooctadecine- |
| | 2,6,9,12,15(3H)-pentaone; |
| 265 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,16-trimethyl-14-((6-methylpyridin-2- |
| | yl)methyl)hexadecahydrobenzo[l][1,4,7,11,14]pentaazacyclooctadecine- |
| | 2,6,9,12,15(3H)-pentaone; |
| 266 | (4S,7S,10S,14R,16aS,20aS)-14-benzyl-11-(4-chloro-2-(4-(5-methyl-4- |
| | (morpholinomethyl)-1H-imidazol-1-yl)phenoxy)benzyl)-4-(4-chlorobenzyl)-7- |
| | (methoxymethyl)-5,10,16- |
| | trimethylhexadecahydrobenzo[l][1,4,7,11,14]pentaazacyclooctadecine- |
| | 2,6,9,12,15(3H)-pentaone; |
| 267 | diethyl 4,4′-((3-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10- |
| | yl)propyl)azanediyl)dibutanoate |
| 268 | (4S,7S,10S,14R,16aS,20aS)-14-benzyl-11-(4-chloro-2-(4-(2-((R)-1- |
| | (dimethylamino)propyl)-1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 269 | 2-((4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-2,6,9,12,15- |
| | pentaoxodocosahydrobenzo[l][1,4,7,11,14]pentaazacyclooctadecin-10-yl)-N,N- |
| | dimethylacetamide |
| 270 | (2S,5S,8S,12S,13S,16R)-16-benzyl-1-(4-chloro-2-(4-(4-((R)-1- |
| | (dimethylamino)ethyl)-5-methyl-1H-imidazol-1-yl)phenoxy)benzyl)-8-(4- |
| | chlorobenzyl)-5-(methoxymethyl)-2,7,12,13,14-pentamethyl-1,4,7,11,14- |
| | pentaazacyclooctadecane-3,6,10,15,18-pentaone; |
| 271 | (2S,5S,8S,13S,16R)-16-benzyl-1-(4-chloro-2-(4-(2-(3,6-dihydro-2H-pyran-4-yl)-1- |
| | methyl-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 272 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(3- |
| | morpholinopropyl)hexadecahydrobenzo[l][1,4,7,11,14]pentaazacyclooctadecine- |
| | 2,6,9,12,15(3H)-pentaone; |
| 273 | (4S,7S,10S,14R,16aS,20aS)-14-benzyl-11-(4-chloro-2-((5-(2- |
| | ((dimethylamino)methyl)-1-methyl-1H-imidazol-5-yl)pyridin-2-yl)oxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 274 | (4S,7S,10S,14R,16aS,20aR)-14-benzyl-11-(4-chloro-2-(4-(1-methyl-2-(pyrrolidin-1- |
| | ylmethyl)-1H-imidazol-5-yl)phenoxy)benzyl)-4-(4-chlorobenzyl)-7-(methoxymethyl)- |
| | 5,10,16-trimethyltetradecahydro-1H-pyrano[3,4- |
| | l][1,4,7,11,14]pentaazacyclooctadecine-2,6,9,12,15(3H)-pentaone; |
| 275 | (4S,7S,10S,16aS,20aS)-11-(4-chloro-2-(4-(2-((dimethylamino)methyl)-1-methyl- |
| | 1H-imidazol-5-yl)phenoxy)benzyl)-4-(4-chlorobenzyl)-7-(methoxymethyl)-5,10,16- |
| | trimethyl-14-((2-oxopyridin-1(2H)- |
| | yl)methyl)hexadecahydrobenzo[l][1,4,7,11,14]pentaazacyclooctadecine- |
| | 2,6,9,12,15(3H)-pentaone; |
| 276 | (4S,7S,10S,16aS,20aS)-11-(4-chloro-2-(4-(2-((dimethylamino)methyl)-1-methyl- |
| | 1H-imidazol-5-yl)phenoxy)benzyl)-4-(4-chlorobenzyl)-7-(methoxymethyl)-5,10,16- |
| | trimethyl-14-((2-oxopyridin-1(2H)- |
| | yl)methyl)hexadecahydrobenzo[l][1,4,7,11,14]pentaazacyclooctadecine- |
| | 2,6,9,12,15(3H)-pentaone; |
| 277 | (4S,7S,10S,14R,16aS,20aS)-14-allyl-11-(4-chloro-2-(4-(2-((dimethylamino)methyl)- |
| | 1-methyl-1H-imidazol-5-yl)phenoxy)benzyl)-4-(4-chlorobenzyl)-7-(methoxymethyl)- |
| | 5,16-dimethyl-10-(3- |
| | morpholinopropyl)hexadecahydrobenzo[l][1,4,7,11,14]pentaazacyclooctadecine- |
| | 2,6,9,12,15(3H)-pentaone; |
| 278 | (4S,7S,10S,14R,16aS,20aS)-14-benzyl-11-(4-chloro-2-(4-(4- |
| | ((dimethylamino)methyl)-5-methyl-1H-imidazol-1-yl)phenoxy)benzyl)-4-(4- |
| | chlorobenzyl)-7-(hydroxymethyl)-5,10,16- |
| | trimethylhexadecahydrobenzo[l][1,4,7,11,14]pentaazacyclooctadecine- |
| | 2,6,9,12,15(3H)-pentaone; |
| 279 | (2S,5S,8S,12S,13S,16R)-1-(4-chloro-2-(4-(1-methyl-2-(pyrrolidin-1-ylmethyl)-1H- |
| | imidazol-5-yl)phenoxy)benzyl)-8-(4-chlorobenzyl)-5-(methoxymethyl)-2,7,12,13,14- |
| | pentamethyl-16-(2,3,4-trifluorobenzyl)-1,4,7,11,14-pentaazacyclooctadecane- |
| | 3,6,10,15,18-pentaone; |
| 280 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,16-trimethyl-14-(pyridin-3- |
| | ylmethyl)hexadecahydrobenzo[l][1,4,7,11,14]pentaazacyclooctadecine- |
| | 2,6,9,12,15(3H)-pentaone; |
| 281 | (2S,5S,8S,13S,16R)-16-benzyl-1-(4-chloro-2-(3-methyl-4-(1-methyl-2-(pyrrolidin-1- |
| | ylmethyl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 282 | (2S,5S,8S,13S,16R)-16-benzyl-1-(4-chloro-2-(4-(1-methyl-2-vinyl-1H-imidazol-5- |
| | yl)phenoxy)benzyl)-8-(4-chlorobenzyl)-5-(methoxymethyl)-2,7,13,14-tetramethyl- |
| | 1,4,7,11,14-pentaazacyclooctadecane-3,6,10,15,18-pentaone; |
| 283 | (2S,5S,8S,12S,13S,16R)-16-benzyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 7,12,13,14-tetramethyl-2-(3-morpholinopropyl)-1,4,7,11,14- |
| | pentaazacyclooctadecane-3,6,10,15,18-pentaone; |
| 284 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-7-(methoxymethyl)- |
| | 5,10,16-trimethyl-14-(pyridin-2- |
| | ylmethyl)hexadecahydrobenzo[l][1,4,7,11,14]pentaazacyclooctadecine- |
| | 2,6,9,12,15(3H)-pentaone; |
| 285 | (4S,7S,10S,14R,16aS,20aS)-14-benzyl-11-(4-chloro-2-(4-(1-methyl-2-(pyrrolidin-1- |
| | ylmethyl)-1H-imidazol-5-yl)phenoxy)benzyl)-4-(4-chlorobenzyl)-10-(3- |
| | (dimethylamino)propyl)-7-(methoxymethyl)-5,16- |
| | dimethylhexadecahydrobenzo[l][1,4,7,11,14]pentaazacyclooctadecine- |
| | 2,6,9,12,15(3H)-pentaone; |
| 286 | (4S,7S,10S,14R,16aS,20aS)-14-(but-2-en-1-yl)-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 287 | tert-butyl 3-((4S,7S,10R,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(hydroxymethyl)-5,16-dimethyl-2,6,9,12,15- |
| | pentaoxodocosahydrobenzo[l][1,4,7,11,14]pentaazacyclooctadecin-10- |
| | yl)propanoate; |
| 288 | (4S,7S,10R,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-10-(2-(dimethylamino)ethyl)-7-(methoxymethyl)-5,16- |
| | dimethylhexadecahydrobenzo[l][1,4,7,11,14]pentaazacyclooctadecine- |
| | 2,6,9,12,15(3H)-pentaone; |
| 289 | (2S,5S,8S,12S,13S,16R)-16-allyl-1-(4-chloro-2-(4-(2-((dimethylamino)methyl)-1- |
| | methyl-1H-imidazol-5-yl)phenoxy)benzyl)-8-(4-chlorobenzyl)-5-(methoxymethyl)- |
| | 2,7,12,13,14-pentamethyl-1,4,7,11,14-pentaazacyclooctadecane-3,6,10,15,18- |
| | pentaone; |
| 290 | (4S,7S,10S,14R,16aS,20aS)-11-(4-chloro-2-(4-(2-((dimethylamino)methyl)-1- |
| | methyl-1H-imidazol-5-yl)phenoxy)benzyl)-4-(4-chlorobenzyl)-14- |
| | (cyclopropylmethyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 291 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-10-(3-(dimethylamino)propyl)-7-(methoxymethyl)-5,16- |
| | dimethylhexadecahydrobenzo[l][1,4,7,11,14]pentaazacyclooctadecine- |
| | 2,6,9,12,15(3H)-pentaone; |
| 292 | (4S,7S,10S,14R,16aS,20aS)-14-benzyl-11-(4-chloro-2-(4-(4- |
| | ((dimethylamino)methyl)-5-methyl-1H-imidazol-1-yl)phenoxy)benzyl)-4-(4- |
| | chlorobenzyl)-7-(methoxymethyl)-5,10,16- |
| | trimethylhexadecahydrobenzo[l][1,4,7,11,14]pentaazacyclooctadecine- |
| | 2,6,9,12,15(3H)-pentaone; |
| 293 | (2S,5S,8S,12S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,12,13,14- |
| | pentamethyl-16-(2,3,4-trifluorobenzyl)-1,4,7,11,14-pentaazacyclooctadecane- |
| | 3,6,10,15,18-pentaone; |
| 294 | (4S,7S,10R,14R,16aS,20aR)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,10,16-trimethyltetradecahydro-1H-pyrano[3,4- |
| | l][1,4,7,11,14]pentaazacyclooctadecine-2,6,9,12,15(3H)-pentaone; |
| 295 | (2S,5S,8S,12S,13S,16R)-1-(4-chloro-2-(4-(2-((dimethylamino)methyl)-1-methyl-1H- |
| | imidazol-5-yl)phenoxy)benzyl)-8-(4-chlorobenzyl)-5-(methoxymethyl)-2,7,12,13,14- |
| | pentamethyl-16-(pyridin-2-ylmethyl)-1,4,7,11,14-pentaazacyclooctadecane- |
| | 3,6,10,15,18-pentaone; |
| 296 | (4S,7S,10S,14R,16aS,20aS)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16-dimethyl-10-(3-(2-oxopyrrolidin-1- |
| | yl)propyl)hexadecahydrobenzo[l][1,4,7,11,14]pentaazacyclooctadecine- |
| | 2,6,9,12,15(3H)-pentaone; |
| 298 | (2S,5S,8S,13S,16R)-16-benzyl-1-(4-chloro-2-(2,6-difluoro-4-(1-methyl-2-(pyrrolidin- |
| | 1-ylmethyl)-1H-imidazol-5-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 299 | (2S,5S,8S,13S,16R)-16-benzyl-1-(4-chloro-2-(2,3-difluoro-4-(1-methyl-2-(pyrrolidin- |
| | 1-ylmethyl)-1H-imidazol-5-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 300 | (2S,5S,8S,13S,16R)-16-benzyl-1-(4-chloro-2-(4-(1-ethyl-2-(pyrrolidin-1-ylmethyl)- |
| | 1H-imidazol-5-yl)phenoxy)benzyl)-8-(4-chlorobenzyl)-5-(methoxymethyl)-2,7,13,14- |
| | tetramethyl-1,4,7,11,14-pentaazacyclooctadecane-3,6,10,15,18-pentaone; |
| 301 | (2S,5S,8S,13S,16R)-16-benzyl-1-(4-chloro-2-(4-(4-methyl-5-(pyrrolidin-1- |
| | ylmethyl)pyridin-3-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; |
| 302 | (2S,5S,8S,13S,16R)-16-benzyl-1-(4-chloro-2-(4-(8-(pyrrolidin-1- |
| | ylmethyl)imidazo[1,2-alpyridin-3-yl)phenoxy)benzyl)-8-(4-chlorobenzyl)-5- |
| | (methoxymethyl)-2,7,13,14-tetramethyl-1,4,7,11,14-pentaazacyclooctadecane- |
| | 3,6,10,15,18-pentaone; |
| 303 | (2S,5S,8S,13S,16R)-16-benzyl-1-(4-chloro-2-(4-(1-(2-fluoroethyl)-2-(pyrrolidin-1- |
| | ylmethyl)-1H-imidazol-5-yl)phenoxy)benzyl)-8-(4-chlorobenzyl)-5-(methoxymethyl)- |
| | 2,7,13,14-tetramethyl-1,4,7,11,14-pentaazacyclooctadecane-3,6,10,15,18- |
| | pentaone; and |
| 304 | (4S,7S,10S,14R,16aS,20aS)-10-(3-aminopropyl)-14-benzyl-11-(4-chloro-2-(4-(2- |
| | ((dimethylamino)methyl)-1-methyl-1H-imidazol-5-yl)phenoxy)benzyl)-4-(4- |
| | chlorobenzyl)-7-(methoxymethyl)-5,16- |
| | dimethylhexadecahydrobenzo[l][1,4,7,11,14]pentaazacyclooctadecine- |
| | 2,6,9,12,15(3H)-pentaone. |

Embodiment 28. The compound (Cmpd) of Embodiment 1 selected from:
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-10-(2-(dimethylamino)ethyl)-7-(methoxymethyl)-5,16-dimethylhexadecahydrobenzo[l][1,4,7,11,14]pentaazacyclooctadecine-2,6,9,12,15(3H)-pentaone; and
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(2-morpholinoethyl)hexadecahydrobenzo[l][1,4,7,11,14]pentaazacyclooctadecine-2,6,9,12,15(3H)-pentaone.

Embodiment 29. A pharmaceutical composition comprising a therapeutically effective amount of a compound according to any one of Embodiments 1-28, or a pharmaceutically acceptable salt thereof, and one or more pharmaceutically acceptable carriers or excipients.

Embodiment 30. A combination comprising a compound according to any one of Embodiments 1-28, or a pharmaceutically acceptable salt thereof, and one or more additional therapeutically active agents.

Embodiment 31. The combination according to Embodiment 30, wherein the additional therapeutically active agent is a statin.

Embodiment 32. The pharmaceutical composition according to Embodiment 29 or combination according to Embodiment 30 or 31, for use in the treatment, prevention, amelioration, or delay in the progression of a PCSK9-mediated disease or disorder.

Embodiment 33. The pharmaceutical composition or the combination according to Embodiment 32, wherein said PCSK9-mediated disease or disorder or the disease or disorder requiring inhibition of PCSK9 activity is selected from hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma.

Embodiment 34. A method of modulating PCSK9 comprising administering to a patient in need thereof a compound of any one of Embodiments 1-28, or a pharmaceutically acceptable salt thereof.

Embodiment 35. A method of inhibiting PCSK9 comprising administering to a patient in need thereof a compound of any one of Embodiments 1-28, or a pharmaceutically acceptable salt thereof.

Embodiment 36. A method for treating, preventing, ameliorating or delaying the progression of a PCSK9-mediated disease or disorder comprising the step of administering to a patient in need thereof a therapeutically effective amount of a compound according to any one of Embodiments 1-28, or a pharmaceutically acceptable salt thereof.

Embodiment 37. The method of Embodiment 36, wherein said PCSK9-mediated disease or disorder is selected from hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma.

Embodiment 38. A method of (i) reducing Lp(a), (ii) reducing Lp(a) plasma levels, (iii) reducing Lp(a) serum levels, (iv) reducing serum TRL or LDL levels, (v) reducing serum triglyceride levels, (vi) reducing LDL-C, (vii) reducing total plasma apoB concentrations, (viii) reducing LDL apoB, (ix) reducing TRL apoB, or (x) reducing non HDL-C, the method comprising administering to a patient in need thereof a therapeutically effective amount of a compound of any one of Embodiments 1-28, or a pharmaceutically acceptable salt thereof to the patient, thereby reducing LDL-C in the patient.

Embodiment 39. The method of Embodiments 34-38, wherein administering is performed orally, parentally, subcutaneously, by injection, or by infusion.

Embodiment 40. A compound according to any one of Embodiments 1-28, or a pharmaceutically acceptable salt thereof, for use in the treatment of a PCSK9-mediated disease or disorder.

Embodiment 41. A compound according to any one of Embodiments 1-28, or a pharmaceutically acceptable salt thereof, for use in the treatment, prevention, amelioration or delay of progression or for use in the treatment, prevention, amelioration or delay of progression of a disease or disorder requiring inhibition of PCSK9.

Embodiment 42. Use of a compound according to any one of Embodiments 1-28, or a pharmaceutically acceptable salt thereof, for the treatment, prevention, amelioration or delay of progression of a PCSK9-mediated disease or disorder or for the treatment, prevention, amelioration or delay of progression of a disease or disorder requiring inhibition of PCSK9.

Embodiment 43. Use of a compound according to any one of Embodiments 1-28, or a pharmaceutically acceptable salt thereof, in the manufacture of a medicament for the treatment, prevention, amelioration or delay of progression of a PCSK9-mediated disease or disorder or for the treatment, prevention, amelioration or delay of progression of a disease or disorder requiring inhibition of PCSK9.

Embodiment 44. A method for treating, preventing, ameliorating or delaying the progression of a PCSK9-mediated disease or disorder or of disease or disorder requiring inhibition of PCSK9 or of PCSK9 activity comprising the step of administering to a patient in need thereof a therapeutically effective amount of a compound according to any one of the Embodiments 1-28, or a pharmaceutically acceptable salt thereof.

Embodiment 45. The compound for use according to Embodiment 41, the use of a compound according to Embodiment 42 or 43, or the method for according to Embodiment 44, wherein said PCSK9-mediated disease or disorder or the disease or disorder requiring inhibition of PCSK9 is selected from hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma.

Embodiment 46. A process for the manufacture of a compound of Formula (II), or a pharmaceutically acceptable salt, hydrate, solvate, stereoisomer, N-oxide, or tautomer thereof,

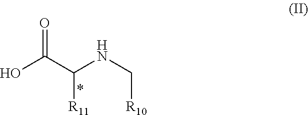

wherein R₁₀ and R₁₁ are as defined above for Formula (I), and * denotes a chiral center, comprising reacting a compound of Formula (IIa), or a pharmaceutically acceptable salt, hydrate, solvate, stereoisomer, N-oxide, or tautomer thereof,

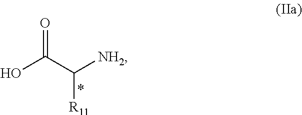

wherein R₁₁ is as defined above for Formula (I) and * denotes a chiral center; with a compound of Formula (IIb), or a pharmaceutically acceptable salt, hydrate, solvate, stereoisomer, N-oxide, or tautomer thereof,

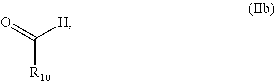

wherein R₁₀ is as defined above for Formula (I), or a pharmaceutically acceptable salt, hydrate, solvate, stereoisomer, N-oxide, or tautomer thereof, via a reductive amination in the presence of a reducing agent, in aliphatic alcohol solvent, and at low temperature, to obtain the compound of Formula (II) in >70% enantiomeric excess (ee).

Embodiment 47. The process of Embodiment 46, wherein the reducing agent is sodium borohydride.

Embodiment 48. The process of Embodiment 46 or 47, wherein the temperature is about −5° C.

Embodiment 49. The process of any one of Embodiments 46-48, wherein the aliphatic alcohol solvent is methanol.

Embodiment 50. The process of any one of Embodiments 46-49, wherein the compound of Formula (II) is obtained in >99% ee.

In another embodiment of the disclosure, the compounds of Formula (I) are enantiomers. In some embodiments the compounds are the (S)-enantiomer. In other embodiments the compounds are the (R)-enantiomer. In yet other embodiments, the compounds of Formula (I) may be (+) or (−) enantiomers.

In another embodiment of the disclosure, the compounds of Formula (I) are diastereomers.

It should be understood that all isomeric forms are included within the present disclosure, including mixtures thereof. If the compound contains a double bond, the substituent may be in the E or Z configuration. If the compound contains a disubstituted cycloalkyl, the cycloalkyl substituent may have a cis- or trans configuration. All tautomeric forms are also intended to be included.

Compounds of the disclosure, and pharmaceutically acceptable salts, hydrates, solvates, stereoisomers and prodrugs thereof may exist in their tautomeric form (for example, as an amide or imino ether). All such tautomeric forms are contemplated herein as part of the present disclosure.

The compounds of the disclosure may contain asymmetric or chiral centers, and, therefore, exist in different stereoisomeric forms. It is intended that all stereoisomeric forms of the compounds of the disclosure as well as mixtures thereof, including racemic mixtures, form part of the present disclosure. In addition, the present disclosure embraces all geometric and positional isomers. For example, if a compound of the disclosure incorporates a double bond or a fused ring, both the cis- and trans-forms, as well as mixtures, are embraced within the scope of the disclosure. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the disclosure may be atropisomers (e.g., substituted biaryls) and are considered as part of this disclosure. Enantiomers can also be separated by use of a chiral HPLC column.

It is also possible that the compounds of the disclosure may exist in different tautomeric forms, and all such forms are embraced within the scope of the disclosure. Also, for example, all keto-enol and imine-enamine forms of the compounds are included in the disclosure.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this disclosure, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the disclosure. Also, for example, all keto-enol and imine-enamine forms of the compounds are included in the disclosure.) Individual stereoisomers of the compounds of the disclosure may, for example, be substantially free of other isomers, or may be admixed, for example, as racemates or with all other, or other selected, stereoisomers. The chiral centers of the present disclosure can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The compounds of Formula (I) may form salts, which are also within the scope of this disclosure. Reference to a compound of the Formula herein is understood to include reference to salts thereof, unless otherwise indicated.

The present disclosure relates to compounds which are modulators of PCSK9. In one embodiment, the compounds of the present disclosure are inhibitors of PCSK9.

The disclosure is directed to compounds as described herein and pharmaceutically acceptable salts, hydrates, solvates, stereoisomers, or tautomers thereof, and pharmaceutical compositions comprising one or more compounds as described herein, or pharmaceutically acceptable salts, hydrates, solvates, stereoisomers, or tautomers thereof.

### Activity of the Compounds

The activity of compounds according to the present disclosure as PCSK9 inhibitors can be assessed using a time resolved fluorescence resonance energy transfer (TR-FRET) assay. This time resolved fluorescence resonance energy transfer (TR-FRET) assay measures the ability of a compounds of the present disclosure to interfere with the binding of human PCSK9 to human LDLR, providing measures of both potency (IC50) and efficacy (Amax).

Solutions of varying concentrations are prepared by diluting a compound of the disclosure in dimethylsulfoxide (DMSO) and the resulting solutions are pipetted into a plate. DMSO is used as a negative control. An intermediate plate is prepared in by transferring a known amount of each compound solution and of the control from the compound plate into a corresponding well containing assay buffer and mixing thoroughly. A third plate is then prepared to be used for the assay by adding Human PCSK9 Alexa Fluor 647, followed by a known amount of each solution from the intermediate plate. Unlabeled human PCSK9 in assay buffer containing DMSO is used as a positive control for the assay. Following incubation, Human LDLR extracellular domain-Europium Kryptate is added to each well of the assay plate and the resulting mixture is incubated for an additional period of time. The TR-FRET signal is measured and the FRET ratio (FRET/Europium) is used to calculate the IC₅₀ and Amax of the compounds.

### Method of Synthesizing the Compounds

The compounds of the present disclosure may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of Formula (I).

Those skilled in the art will recognize if a stereocenter exists in the compounds of Formula (I). Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present disclosure can be prepared in a number of ways well known to those skilled in the art of organic synthesis. By way of example, compounds of the present disclosure can be synthesized using the methods described below, together with synthetic methods known in the art of synthetic organic chemistry, or variations thereof as appreciated by those skilled in the art. Preferred methods include but are not limited to those methods described below. Compounds of the present disclosure can be synthesized by following the steps outlined in General Scheme 1 which comprise different sequences of assembling intermediates (1-a)-(1-k) and 1-L. Starting materials are either commercially available or made by known procedures in the reported literature or as illustrated.

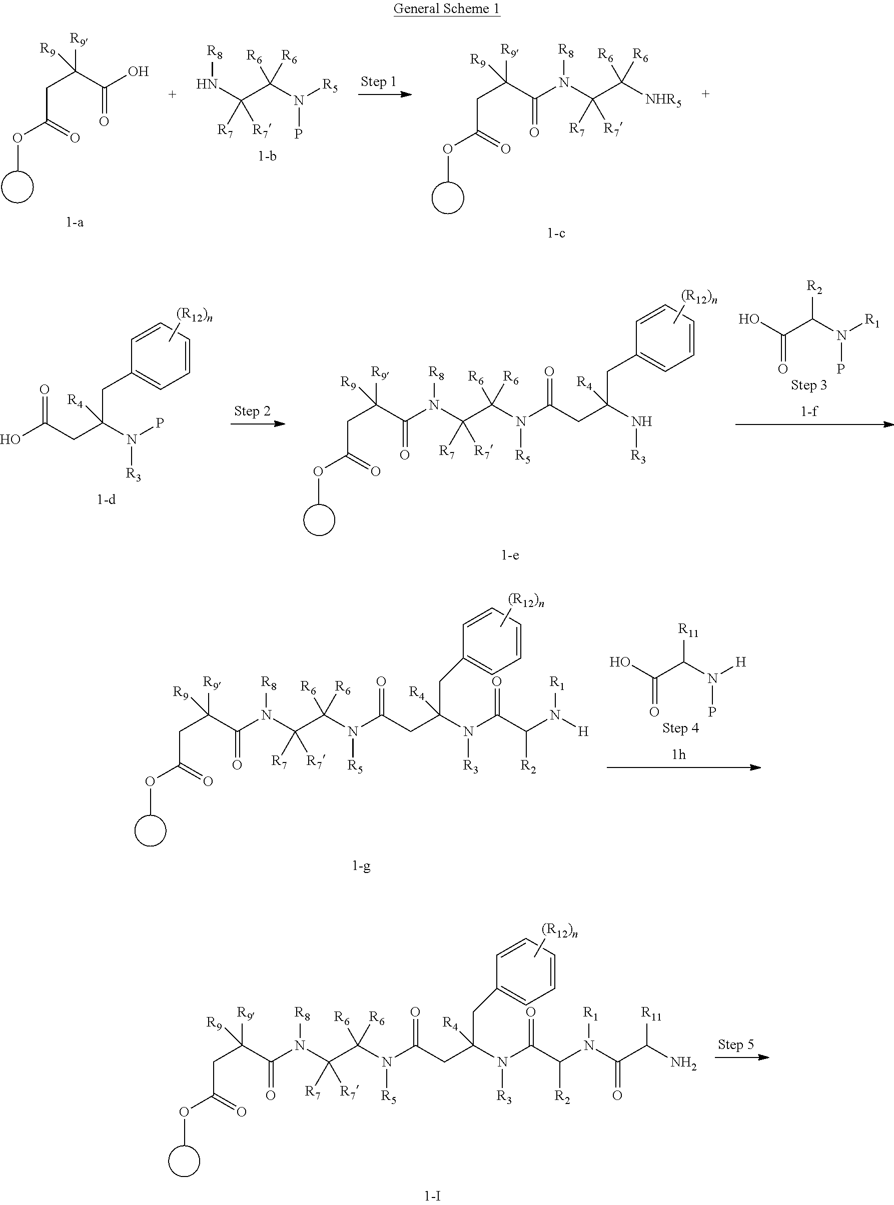

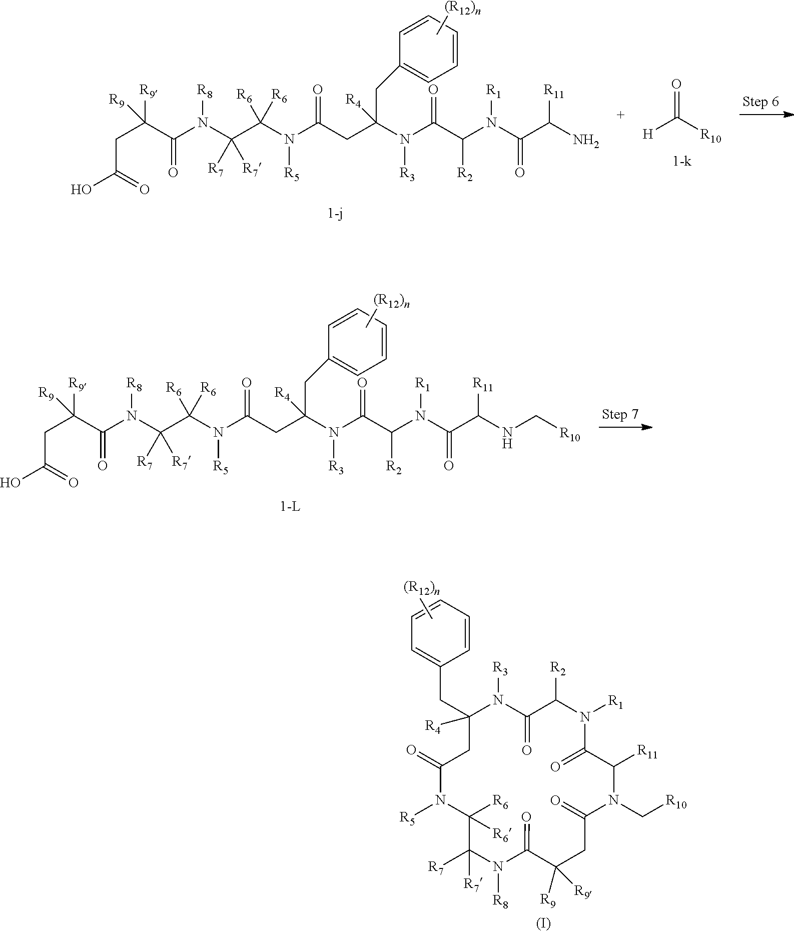

wherein R₁, R₂, R₃, R₄, R₅, R₆, R_{6′}, R₇, R_{7′}, R₈, R₉, R_{9′}, R₁₀, R₁₁, R₁₂, and n are defined in Formula (I) and P is an amine protecting group (e.g., tert-Butyloxycarbonyl (Boc), 9-Fluorenylmethyloxycarbonyl (Fmoc), etc.).

The general way of preparing compounds of Formula (I) wherein X₁ is C by using intermediates 1-a, 1-b, 1-c, 1-d, 1-e, 1-f, 1-g, 1-h, 1-i, 1-j, 1-k, and 1-L is outlined in General Scheme 1. Synthesis of intermediate 1-c can be accomplished by coupling of 1-a with 1-b under standard coupling conditions using an amide coupling reagent (e.g., 2-(1H-Benzotriazol-1-yl)-1,1,3,3-tetramethyluronium-tetrafluoroborate (TBTU), O-(1H-6-chlorobenzotriazole-1-yl)-1,1,3,3-tetramethyluronium hexafluorophosphate (HCTU), or O-(7-azobenzotriazol-1-yl)-1,1,3,3-tetramethyluroniumhexafluorophosphate (HATU) and optionally a base (e.g., triethylamine (TEA) or N,N-diisopropylethylamine (DIPEA)) in a solvent (e.g., N-Methylpyrrolidine (NMP) or dimethylformamide (DMF)) on a resin (e.g., TentaGel™ S RAM resin) followed by removal of amine protection group P (e.g., treatment with 4-methylpiperidine/DMA for removal of Fmoc group). Coupling of 1-c and acid 1-d using an amide coupling reagent (e.g., TBTU, HCTU, or HATU) and optionally a base (e.g., triethylamine or N,N-diisopropylethylamine (DIPEA)) followed by deprotection (e.g., treatment with 4-methylpiperidine/DMA for removal of Fmoc group) provides 1-e. The coupling and deprotection steps are repeated in steps 3 and 4 using the standard coupling conditions and deprotection conditions described above to provide intermediate 1-i.

Cleavage of 1-i from the resin by repetitive treatment with 1,1,1,3,3,3-Hexafluoropropan-2-ol (HFIP) in a solvent (e.g., dichloromethane (DCM)) provides 1-j. Reductive amination of amine 1-j and aldehyde 1-k in the presence of a reducing agent, e.g., sodium triacetoxyborohydride, sodium cyanoborohydride, or sodium borohydride, optionally an acid (e.g., acetic acid (AcOH)) in a solvent, e.g., methanol (MeOH) and/or DCM, provides 1-L. Cyclization of 1-L using standard coupling conditions, e.g., an amide coupling reagent (e.g., HATU, HOAt, TBTU, and/or HCTU), optionally a base (e.g., 2,6-lutidine, TEA or DIPEA) in a solvent (e.g., DCM, NMP or DMF) provides the desired compound of Formula (I).

It should be understood that in the description and formula shown above, the various groups X₁, R₁, R₂, R₃, R₄, R₅, R₆, R_{6′}, R₇, R_{7′}, R₈, R₉, R_{9′}, R₁₀, R₁₁, R₁₂, and n, and other variables are as defined above, except where otherwise indicated. Furthermore, for synthetic purposes, the compounds of General Scheme 1 are mere representative with elected radicals to illustrate the general synthetic methodology of the compounds of Formula (I) as defined herein.

### Methods of Using the Disclosed Compounds

Another aspect of the disclosure is directed to a method of modulating PCSK9. The method comprises administering to a patient in need thereof an effective amount of a compound of Formula (I), or a pharmaceutically acceptable salt, hydrate, solvate, stereoisomer, or tautomer thereof or a pharmaceutical composition comprising a compound of Formula (I), or a pharmaceutically acceptable salt, hydrate, solvate, stereoisomer, or tautomer thereof, and a pharmaceutically acceptable carrier.

In another aspect, the disclosure is directed to a method of inhibiting PCSK9. The method involves administering to a patient in need thereof an effective amount of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier.

Another aspect of the disclosure relates to a method of treating, preventing, inhibiting, or eliminating a disease or disorder in which PCSK9 plays a role. The method comprises administering to a patient in need of a treatment for diseases or disorders in which PCSK9 plays a role an effective amount of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier.

Another aspect of the present disclosure relates to a method of treating, preventing, inhibiting, or eliminating a disease or disorder in a patient associated with the inhibition of PCSK9, the method comprising administering to a patient in need thereof an effective amount of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier.

In another aspect, the present disclosure relates to a method of treating, preventing, inhibiting, or eliminating a PCSK9-mediated disease or disorder. The method comprises administering to a patient in need of a treatment for a PCSK9-mediated disease or disorder an effective amount of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier.

Another aspect of the present disclosure relates to a method of treating, preventing, inhibiting, or eliminating hypercholesterolemia, hyperlipidemia, hypertriglyceridemia, sitosterolemia, atherosclerosis, arteriosclerosis, coronary heart disease, peripheral vascular disease, vascular inflammation, xanthoma, peripheral arterial disease, sepsis, elevated Lp(a), elevated LDL, elevated TRL, or elevated triglycerides. The method comprises administering to a patient in need of a treatment an effective amount of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier.

In another aspect, the present disclosure relates to a method of reducing Lp(a), reducing Lp(a) plasma levels, reducing Lp(a) serum levels, reducing serum TRL or LDL levels, reducing serum triglyceride levels, reducing LDL-C, reducing total plasma apoB concentrations, reducing LDL apoB, reducing TRL apoB, or reducing non HDL-C. The method comprises administering to a patient in need thereof an effective amount of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier.

In another aspect, the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the treatment, prevention, inhibition, or elimination of a disease or disorder in which PCSK9 plays a role.

Another aspect of the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the treatment, prevention, inhibition, or elimination of a disease associated with inhibiting PCSK9.

Another aspect of the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the treatment, prevention, inhibition, or elimination of hypercholesterolemia, hyperlipidemia, hypertriglyceridemia, sitosterolemia, atherosclerosis, arteriosclerosis, coronary heart disease, peripheral vascular disease, vascular inflammation, xanthoma, peripheral arterial disease, sepsis, elevated Lp(a), elevated LDL, elevated TRL, or elevated triglycerides.

In another aspect, the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the reduction of Lp(a), in the reduction of Lp(a) plasma levels, in the reduction of Lp(a) serum levels, in the reduction of serum TRL or LDL levels, in the reduction of serum triglyceride levels, in the reduction of LDL apoB, in the reduction of TRL apoB, or in the reduction of non HDL-C.

Another aspect of the present disclosure relates to the use of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for treating, preventing, inhibiting, or eliminating a disease or disorder in which PCSK9 plays a role.

In another aspect, the present disclosure relates to the use of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier in the manufacture of a medicament for inhibiting PCSK9.

In another aspect, the present disclosure relates to the use of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier in the manufacture of a medicament for treating, preventing, inhibiting, or eliminating hypercholesterolemia, hyperlipidemia, hypertriglyceridemia, sitosterolemia, atherosclerosis, arteriosclerosis, coronary heart disease, peripheral vascular disease, vascular inflammation, xanthoma, peripheral arterial disease, sepsis, elevated Lp(a), elevated LDL, elevated TRL, or elevated triglycerides.

Another aspect of the present disclosure relates to the use of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier in the manufacture of a medicament for reducing Lp(a), reducing Lp(a) plasma levels, reducing Lp(a) serum levels, reducing serum TRL or LDL levels, reducing serum triglyceride levels, reducing LDL apoB, reducing TRL apoB, or reducing non HDL-C.

In another aspect, the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the manufacture of a medicament for treating a disease associated with inhibiting PCSK9.

Another aspect of the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the manufacture of a medicament for treating a disease in which PCSK9 plays a role.

In another aspect, the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the manufacture of a medicament for treating, preventing, inhibiting, or eliminating hypercholesterolemia, hyperlipidemia, hypertriglyceridemia, sitosterolemia, atherosclerosis, arteriosclerosis, coronary heart disease, peripheral vascular disease, vascular inflammation, xanthoma, peripheral arterial disease, sepsis, elevated Lp(a), elevated LDL, elevated TRL, or elevated triglycerides.

Another aspect of the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the manufacture of a medicament for reducing Lp(a), reducing Lp(a) plasma levels, reducing Lp(a) serum levels, reducing serum TRL or LDL levels, reducing serum triglyceride levels, reducing LDL apoB, reducing TRL apoB, or reducing non HDL-C.

In another aspect, the present disclosure relates to the use of an inhibitor of PCSK9 for the preparation of a medicament used in the treatment, prevention, inhibition or elimination of hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, or xanthoma.

Another aspect of the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the treatment of a PCSK9-mediated disease or disorder.

Another aspect of the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the treatment of a PCSK9-mediated disease or disorder which is selected from hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma.

In another aspect, the present disclosure relates to the use of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier in the manufacture of a medicament for treating a PCSK9-mediated disease or disorder.

Another aspect of the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use in the manufacture of a medicament for treating a PCSK9-mediated disease or disorder.

In another aspect, the present disclosure relates to the use of a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, and a pharmaceutically acceptable carrier for use as a medicament.

The present disclosure also relates to the use of an inhibitor of PCSK9 for the preparation of a medicament used in the treatment, prevention, inhibition, or elimination of a disease or condition in which PCSK9 plays a role, wherein the medicament comprises a compound of Formula (I).

In another aspect, the present disclosure relates to a method for the manufacture of a medicament for treating, preventing, inhibiting, or eliminating a disease or condition mediated by PCSK9, wherein the medicament comprises a compound of Formula (I), or a pharmaceutically acceptable salt, hydrate, solvate, stereoisomer, or tautomer, or a pharmaceutical composition comprising a compound of Formula (I), or a pharmaceutically acceptable salt, hydrate, solvate, stereoisomer, or tautomer thereof, and a pharmaceutically acceptable carrier.

In some embodiments of the methods above, the PCSK9-mediated disease or disorder, the disease or disorder in which PCSK9 plays a role, the disease or disorder in a patient associated with the inhibition of PCSK9, and the disease associated with inhibiting PCSK9 is selected from hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma.

The compounds of the present disclosure find use in reducing or lowering low density lipoprotein cholesterol (LDL-C) in an individual in need thereof. The individual may have persistently elevated levels of LDL-C. In some embodiments, the individual has LDL-C plasma levels consistently above 70 mg/dL, for example above 80, 90, 100, 110, 120, 130, 140, 150, 160, 170, 180, or 190 mg/dL, or higher. The compounds of the present disclosure may also be used to reduce or lower non-high density lipoprotein cholesterol (non-HDL-C) or total cholesterol in an individual in need thereof.

The present disclosure also related to methods for improving blood cholesterol markers associated with increased risk of heart disease. These markers include high total cholesterol, high LDL, high total cholesterol to HDL ratio and high LDL to HDL ratio. A total cholesterol of less than 200 mg/dL is considered desirable, 200-239 mg/dL is considered borderline high and 240 mg/dL and above is considered high.

In a further aspect, the disclosure provides methods of reducing LDL-C, non-HDL-C and/or total cholesterol in an individual in need thereof, the method comprising administering a therapeutically effective amount to the individual a compound as described herein.

In another embodiment, the present disclosure relates to a compound of Formula (I), or a pharmaceutically acceptable salt, hydrate, solvate, stereoisomer, or tautomer thereof, or a pharmaceutical composition comprising a compound of Formula (I), or a pharmaceutically acceptable salt, hydrate, solvate, stereoisomer, or tautomer thereof, of the present disclosure and a pharmaceutically acceptable carrier used for the treatment of diseases including, but not limited to, hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma.

In one embodiment, are provided methods of treating a disease or disorder in which PCSK9 plays a role including hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma comprising administering to a patient suffering from at least one of said diseases or disorder a compound of Formula (I).

The disclosed compounds can be administered in effective amounts to treat or prevent a disorder and/or prevent the development thereof in subjects.

The disclosed compounds can be administered in effective amounts to treat or prevent a disorder and/or prevent the development thereof in subjects.

### Administration, Pharmaceutical Compositions, and Dosing of the Disclosed Compounds

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Another aspect of the disclosure is directed to pharmaceutical compositions comprising a compound of Formula (I), or a pharmaceutically acceptable salt, hydrate, solvate, stereoisomer, or tautomer thereof, and a pharmaceutically acceptable carrier. The pharmaceutical acceptable carrier may further include an excipient, diluent, or surfactant. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. The pharmaceutical compositions of the present disclosure can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and

e) absorbents, colorants, flavors and sweeteners.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, of the disclosed pharmaceutical compositions, or of the disclosed combinations, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, between about 0.1-500 mg/kg, or between about 1-100 mg/kg.

### Combination Therapy

The compounds of the disclosure can be administered in therapeutically effective amounts in a combinational therapy with one or more therapeutic agents (pharmaceutical combinations) or modalities, e.g., non-drug therapies. For example, synergistic effects can occur with other cardiovascular agents, antihypertensive agents, coronary vasodilators, and diuretic substances. Where the compounds of the application are administered in conjunction with other therapies, dosages of the co-administered compounds will of course vary depending on the type of co-drug employed, on the specific drug employed, on the condition being treated and so forth.

The compound of the present disclosure may be administered either simultaneously with, or before or after, one or more other therapeutic agent. The compound of the present disclosure may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the present disclosure.

In one embodiment, the disclosure provides a product comprising a compound of the present disclosure and at least one other therapeutic agent as a combined preparation for simultaneous, separate or sequential use in therapy. In one embodiment, the therapy is the treatment of a disease or condition mediated by PCSK9. Products provided as a combined preparation include a composition comprising the compound of the present disclosure and the other therapeutic agent(s) together in the same pharmaceutical composition, or the compound of the present disclosure and the other therapeutic agent(s) in separate form, e.g. in the form of a kit.

In another aspect, the disclosure includes a compound of Formula (I), Formula (Ia), Formula (Ia-1), Formula (Ia-2), Formula (Ib), Formula (Ic), Formula (Id), Formula (Ie), Formula (If), Formula (Ig), Formula (Ih), Formula (Ii), Formula (Ij), a compound according to any one of embodiment No. 1 to No. 28, or any embodiment of Formula (I), Formula (Ia), Formula (Ia-1), Formula (Ia-2), Formula (Ib), Formula (Ic), Formula (Id), Formula (Ie), Formula (If), Formula (Ig), Formula (Ih), Formula (Ii), Formula (Ij) described herein, or a pharmaceutically acceptable salt thereof, for use in a combination therapy. A compound, composition, medicament and compounds for use of F Formula (I), Formula (Ia), Formula (Ia-1), Formula (Ia-2), Formula (Ib), Formula (Ic), Formula (Id), Formula (Ie), Formula (If), Formula (Ig), Formula (Ih), Formula (Ii), Formula (Ij), a compound according to any one of embodiment No. 1 to No. 28, or any embodiment of Formula (I), Formula (Ia), Formula (Ia-1), Formula (Ia-2), Formula (Ib), Formula (Ic), Formula (Id), Formula (Ie), Formula (If), Formula (Ig), Formula (Ih), Formula (Ii), Formula (Ij) described herein, or a pharmaceutically acceptable salt thereof, may also be used to advantage in combination with one or more other therapeutic agents.

Another aspect of the disclosure is directed to pharmaceutical compositions comprising a compound of Formula (I), or a pharmaceutically acceptable salt, hydrate, solvate, stereoisomer, or tautomer thereof, a pharmaceutically acceptable carrier, and one or more therapeutic agents. The pharmaceutical acceptable carrier may further include an excipient, diluent, or surfactant.

Combination therapy includes the administration of the subject compounds in further combination with other biologically active ingredients (such as, but not limited to, a second agent such as, but not limited to, a cardiovascular agent, an adrenergic blocker, an antihypertensive agent, an angiotensin system inhibitor, an angiotensin-converting enzyme (ACE) inhibitor, a coronary vasodilator, a diuretic, or an adrenergic stimulant or a second agent that targets PCSK9) and non-drug therapies (such as, but not limited to, surgery or radiation treatment). For instance, the compounds of the application can be used in combination with other pharmaceutically active compounds, preferably compounds that are able to enhance the effect of the compounds of the application. The compounds of the application can be administered simultaneously (as a single preparation or separate preparation) or sequentially to the other drug therapy or treatment modality. In general, a combination therapy envisions administration of two or more drugs during a single cycle or course of therapy.

In some embodiments, compounds of the application can be used in combination with agents known to be beneficial for reducing cholesterol, including LDL-C, non-HDL-C, triglyceride-lowering agents, and total cholesterol and/or raising HDL-C.

Exemplary therapeutic agents that may be used in combination with the compounds of the disclosure, include, but are not limited to, hypolipidemic agents, niacin and analogs thereof, bile acid sequestrants, a thyroid hormone mimetic, thyroid hormone receptor (THR) β-selective agonist, a microsomal triglyceride transfer protein (MTP) inhibitor, an acyl CoA:diacylglycerol acyltransferase 1 (DGAT1) inhibitor, a Niemann Pick C1-like 1 (NPC1-L1) inhibitor, an agonist of ATP Binding Cassette (ABC) proteins G5 or G8, an inhibitory nucleic acid targeting PCSK9, an inhibitory nucleic acid targeting apoB100, apoA-I up-regulator/inducer, ABCA1 stabilizer or inducer, phospholipid transfer protein (PLTP) inhibitor, fish oil, anti-diabetic agent, anti-obesity agent, agonists of peroxisome proliferator-activator receptors, ATP citrate lyase (ACL) inhibitor, and anti-hypertensive agents.

Examples of hypolipidemic agents that may be used in combination with the compounds of the disclosure include, but are not limited to, an HMG-CoA reductase inhibitor, squalene synthase inhibitors, LXR agonist, FXR agonist, fibrates, cholesterol absorption inhibitors, nicotinic acid bile acid binding resins, nicotinic acid and other GPR109 agonists and aspirin.

HMG-CoA reductase inhibitors (i.e., statins) are a class of drugs used to lower cholesterol levels by inhibiting the enzyme HMG-CoA reductase, which plays a central role in the production of cholesterol in the liver. Increased cholesterol levels have been associated with cardiovascular diseases and statins are therefore used in the prevention of these diseases. Exemplary statins include, but are not limited to, atorvastatin, cerivastatin, compactin, dalvastatin, dihydrocompactin, fluindostatin, fluvastatin, lovastatin, pitavastatin, mevastatin, pravastatin, rivastatin, simvastatin, and velostatin, or pharmaceutically acceptable salts thereof.

Fibrates or fibric acid derivatives lower triglycerides and raise HDL cholesterol. They may have little effect on LDL cholesterol. For example, Gemfibrozil or fenofibrate is prescribed for people who have very high triglycerides or who have low HDL and high triglycerides. Gemfibrozil may be used to reduce the risk of heart attack in people with coronary artery disease (CAD) who have low HDL and high triglycerides. Examples of fibrates include, but are not limited to, clofibrate, gemfibrozil, fenofibrate, ciprofibrate, and bezafibrate.

Cholesterol absorption inhibitors are a class of compounds that prevents the uptake of cholesterol from the small intestine into the circulatory system, and, in turn, reduce plasma LDL-C concentrations. Increased cholesterol levels are associated with increased CVD risk; thus, cholesterol absorption inhibitors are used with the goal of reducing CVD risk. A non-limiting example of a cholesterol absorption inhibitor is Ezetimibe, previously known as “Sch-58235”. Another example is Sch-48461. Both compounds are developed by Schering-Plough.

Examples of bile acid sequestrants that may be used in combination with the compounds of the disclosure include, but are not limited to, cholestyramine, colestipol, and colesvelam.

A non-limiting example of a thyroid hormone mimetic that may be used in combination with the compounds of the disclosure is compound KB2115.

A non-limiting example of a thyroid hormone receptor (THR) β-selective agonist that may be used in combination with the compounds of the disclosure is MGL-3196.

DGAT is an enzyme that catalyzes the last step in triacylglycerol biosynthesis. DGAT catalyzes the coupling of a 1,2-diacylglycerol with a fatty acyl-CoA resulting in Coenzyme A and triacylglycerol. Two enzymes that display DGAT activity have been identified: DGAT1 (acyl coA-diacylglycerol acyl transferase 1, see Cases et al, Proc. Natl. Acad. Sci. 95:13018-13023, 1998) and DGAT2 (acyl coA-diacylglycerol acyl transferase 2, see Cases et al, J. Biol. Chem. 276:38870-38876, 2001). DGAT1 and DGAT2 do not share significant protein sequence homology. Importantly, DGAT1 knockout mice are protected from high fat diet-induced weight gain and insulin resistance (Smith et al, Nature Genetics 25:87-90, 2000). The phenotype of the DGAT1 knockout mice suggests that a DGAT1 inhibitor has utility for the treatment of obesity and obesity-associated complications. DGAT1 inhibitors useful in said combination are compounds and analogs generically and specifically disclosed e.g. in WO2007/126957 and WO2009/040410, in particular in the compound claims and the final products of the working examples, the subject-matter of the final products, the pharmaceutical preparations and the claims.

Examples of DGAT1 inhibitors suitable for use in combination with compounds of the present disclosure, include but are not limited to, {4-[4-(3-Methoxy-5-phenylamino-pyridin-2-yl)-phenyl]-cyclohexyl}-acetic acid, (4-{4-[5-(1-Methyl-1H-pyrazol-3-ylamino)-pyridin-2-yl]-phenyl}-cyclohexyl)-acetic acid, (4-{4-[5-(5-Fluoro-6-methoxy-pyridin-3-ylamino)-pyridin-2-yl]-phenyl}-cyclohexyl)-acetic acid, (4-{5-[5-(6-Trifluoromethyl-pyridin-3-ylamino)-pyridin-2-yl]-spirocyclohexylidenyl-1,1′-indanyl}-acetic acid, (4-{4-[5-(Benzooxazol-2-ylamino)-pyridin-2-yl]-phenyl}-cyclohexyl)-acetic acid, 4-(4-{4-[2-(3-Chlorophenylamino)-oxazol-5-yl]-phenyl}-cyclohexyl)-butyric acid, (4-{4-[5-(6-Trifluoromethyl-pyridin-3-ylamino)-pyridin-2-yl]-phenyl}-cyclohexyl)-acetic acid, (6-{4-[4-(2H-Tetrazol-5-ylmethyl)-cyclohexyl]-phenyl}-pyridazin-3-yl)-(6-trifluoromethyl-pyridin-3-yl)-amine, 3-(4-{4-[6-(6-Trifluoromethyl-pyridin-3-ylamino)-pyridazin-3-yl]-phenyl}-cyclohexylmethyl)-4H-[1,2,4]oxadiazol-5-one, (1-{4-[6-(3-Trifluoromethyl-phenylamino)-pyridazin-3-yl]-phenyl}-piperidin-4-yl)-acetic acid, (4-{4-[4-Methyl-6-(6-trifluoromethyl-pyridin-3-ylamino)-pyridazin-3-yl]-phenyl}-cyclohexyl)-acetic acid, (4-{4-[5-(6-Trifluoromethyl-pyridin-3-ylamino)-pyrazin-2-yl]-phenyl}-cyclohexyl)-acetic acid, 6-[5-(4-Chloro-phenyl)-[1,3,4]oxadiazol-2-yl]-2-(2,6-dichloro-phenyl)-1H-benzoimidazole, 6-(5-Cyclohexyl-[1,3,4]oxadiazol-2-yl)-2-(2,6-dichloro-phenyl)-1H-benzoimidazole, 6-(5-Butyl-[1,3,4]oxadiazol-2-yl)-2-(2,6-dichloro-phenyl)-1H-benzoimidazole, 2-(2,6-Dichloro-phenyl)-6-[5-(5-methyl-pyridin-3-yl)-[1,3,4]oxadiazol-2-yl]-1H-benzoimidazole, 6-[5-(4-Chloro-phenyl)-[1,3,4]oxadiazol-2-yl]-2-(2,6-dimethyl-4-morpholin-4-yl-phenyl)-1H-benzoimidazole, 6-[5-(4-Chloro-phenyl)-[1,3,4]oxadiazol-2-yl]-2-(3,5-dichloro-pyridin-4-yl)-1H-benzoimidazole, 3-(4-{5-[5-(4-Methoxy-phenyl)-[1,3,4]oxadiazol-2-yl]-1H-benzoimidazol-2-yl}-3,5-dimethyl-phenyl)-2,2-dimethyl-propionic acid, 3-(4-{6-[5-(4-Methoxy-phenyl)-[1,3,4]oxadiazol-2-yl]-1H-benzoimidazol-2-yl}-3,5-dimethyl-phenyl)-propionic acid, 3-(4-{6-[5-(4-methoxyphenylamino)-[1,3,4]oxadiazol-2-yl]-1H-benzimidazol-2-yl}-3,5-dimethylphenyl)-propionic acid, [3-(4-{6-[5-(4-Chloro-phenyl)-[1,3,4]oxadiazol-2-yl]-1H-benzoimidazol-2-yl}-3,5-dimethyl-phenyl)-propyl]-phosphonic acid, 2-(2,6-Dichloro-phenyl)-6-(4,5-diphenyl-oxazol-2-yl)-1H-benzoimidazole, (4-{6-[5-(4-Chloro-phenyl)-[1,3,4]oxadiazol-2-yl]-1H-benzoimidazol-2-yl}-3,5-dimethyl-phenoxy)-acetic acid, 2-(2,6-Dichloro-phenyl)-6-(5-pyrrolidin-1-yl-[1,3,4]oxadiazol-2-yl)-1H-benzoimidazole, and 3,5-Dimethyl-4-{6-[5-(4-trifluoromethyl-phenylamino)-[1,3,4]oxadiazol-2-yl]-1H-benzoimidazol-2-yl}-phenol.

A non-limiting example of a Niemann Pick C1-like 1 (NPC1-L1) inhibitor that may be used in combination with the compounds of the disclosure is ezetimibe.

Apolipoprotein A-I is a protein that in humans is encoded by the APOA1 gene. It has a specific role in lipid metabolism. Apolipoprotein A-I is the major protein component of high density lipoprotein (HDL) in plasma. Chylomicrons secreted from enterocytes also contain ApoA-I but it is quickly transferred to HDL in the bloodstream. The protein promotes cholesterol efflux from tissues to the liver for excretion. It is a cofactor for lecithin cholesterolacyltransferase (LCAT) which is responsible for the formation of most plasma cholesteryl esters. Infusion of a variant of apoA-I in humans has been shown to regress atherosclerotic plaque, as assessed by intravascular ultrasound; thus, apoA-I reduces CVD risk and has the ability to both slow progression and induce regression of atherosclerosis. A non-limiting example of an apoA-I up-regulator/inducer is RVX208.

ATP-binding cassette transporter, ABCA1 (member 1 of human transporter sub-family ABCA), also known as the cholesterol efflux regulatory protein (CERP) is a protein which in humans is encoded by the ABCA1 gene. This transporter is a major regulator of cellular cholesterol and phospholipid homeostasis. A non-limiting example of an ABCA1 regulator is Probucol. Probucol lowers the level of cholesterol in the bloodstream by increasing the rate of LDL catabolism. Additionally, probucol may inhibit cholesterol synthesis and delay cholesterol absorption. Probucol is a powerful antioxidant, which inhibits the oxidation of cholesterol in LDLs; this slows the formation of foam cells, which contribute to atherosclerotic plaques.

The liver X receptor (LXR) is a member of the nuclear receptor family of transcription factors and is closely related to nuclear receptors such as PPAR, FXR and RXR. Liver X receptors (LXRs) are important regulators of cholesterol, fatty acids and glucose homeostasis. LXR agonists are effective for treatment of murine models of atherosclerosis, diabetes, anti-inflammation and Alzheimer's disease. Treatment with LXR agonists (including but not limited to, hypocholamide, T0901317, GW3965, or N,N-dimethyl-3-beta-hydroxy-cholenamide (DMHCA)) lowers the cholesterol level in serum and liver and inhibits the development of atherosclerosis in murine disease models. Examples of LXR agonists include, but are not limited to, GW3965 (a synthetic nonsteroidal liver X receptor (LXR) agonist/activator) and T0901317 (a dual LXR, FXR agonist).

The farnesoid X receptor (FXR), also known as NR1H4 (nuclear receptor subfamily 1, group H, member 4) is a nuclear hormone receptor with activity similar to that seen in other steroid receptors such as estrogen or progesterone but more similar in form to PPAR, LXR and RXR. Activation of the nuclear receptor FXR is known to improve hyperglycemia and hyperlipidemia. A non-limiting example of a FXR agonist is GW4064 (3-(2,6-Dichlorophenyl)-4-(3′-carboxy-2-chlorostilben-4-yl)oxymethyl-5-isopropylisoxazole).

Phospholipid transfer protein (PLTP) is a protein that in humans is encoded by the PLTP gene. The protein encoded by this gene is one of at least two lipid transfer proteins found in human plasma, with CETP being the other. The encoded protein transfers phospholipids from triglyceride-rich lipoproteins to HDL. In addition to regulating the size of HDL particles, this protein may be involved in cholesterol metabolism. At least two transcript variants encoding different isoforms have been found for this gene. Because PLTP influences the metabolism of both triglyceride-rich lipoproteins and HDL, modulation of this transfer protein has the potential to alter cardiovascular disease risk.

Fish oil is derived from the tissues of oily fish. Fish oils contain the omega-3 fatty acids eicosapentaenoic acid (EPA) and docosahexaenoic acid (DHA), precursors of eicosanoids that are known to have many health benefits. Fish oil and other omega-3 sources are most highly recommended for the following conditions: hypertriglyceridemia, secondary cardiovascular disease and prevention of high blood pressure. For example, Lovaza® is used along with a low-fat and low-cholesterol diet to lower very high triglycerides (fats) in your blood. Examples of omega-3 fatty acids that may be used in combination with the compounds of the disclosure include, but are not limited to Lovaza® and Vascepa® (icosapent ethyl).

Examples of anti-diabetic agents that may be used in combination with the compounds of the disclosure include, but are not limited to, insulin, insulin derivatives and mimetics; insulin secretagogues such as the sulfonylureas; insulinotropic sulfonylurea receptor ligands such as meglitinides, e.g., nateglinide and repaglinide; protein tyrosine phosphatase-1B (PTP-1B) inhibitors including, but not limited to, PTP-112; GSK3 (glycogen synthase kinase-3) inhibitors including, but not limited to, SB-517955, SB-4195052, SB-216763, NN-57-05441 and NN-57-05445; RXR ligands including, but not limited to, GW-0791 and AGN-194204; sodium-dependent glucose cotransporter inhibitors including, but not limited to, T-1095; glycogen phosphorylase A inhibitors including, but not limited to, BAY R3401; biguanides including, but not limited to, metformin; alpha-glucosidase inhibitors including, but not limited to, acarbose; GLP-1 (glucagon like peptide-1), GLP-1 analogs including, but not limited to, Exendin-4 and GLP-1 mimetics; and DPPIV (dipeptidyl peptidase IV) inhibitors including, but not limited to, vildagliptin.

Examples of sulfonylureas include, but are not limited to, tolbutamide, chlorpropamide, tolazamide, acetohexamide, 4-chloro-N-[(1-pyrolidinylamino)carbonyl]-benzenesulfonamide (glycopyramide), glibenclamide (glyburide), gliclazide, 1-butyl-3-metanilylurea, carbutamide, glibonuride, glipizide, gliquidone, glisoxepid, glybuthiazole, glibuzole, glyhexamide, glymidine, glypinamide, phenbutamide, amaryl, and tolylcyclamide, or pharmaceutically acceptable salts thereof.

DPP-IV (dipeptidyl peptidase IV) is responsible for inactivating GLP-1. More particularly, DPP-IV generates a GLP-1 receptor antagonist and thereby shortens the physiological response to GLP-1. GLP-1 is a major stimulator of pancreatic insulin secretion and has direct beneficial effects on glucose disposal.

The DPP-IV inhibitor can be peptidic or, preferably, non-peptidic. Examples of DPP-IV inhibitors also include, but are not limited to, generically and specifically DPP-IV inhibitors disclosed in WO 98/19998, DE 196 16 486 A1, WO 00/34241 and WO 95/15309, in each case in particular in the compound claims and the final products of the working examples, the subject-matter of the final products, the pharmaceutical preparations and the claims are hereby incorporated into the present application by reference to these publications.

GLP-1 (glucagon like peptide-1) is an insulinotropic protein which is described, e.g., by W. E. Schmidt et al. in Diabetologia, 28, 1985, 704-707 and in U.S. Pat. No. 5,705,483. The term “GLP-1 agonists” includes variants and analogs of GLP-1(7-36)NH₂ which are disclosed in particular in U.S. Pat. Nos. 5,120,712, 5,118,666, 5,512,549, WO 91/11457 and by C. Orskov, et al, in J. Biol. Chem., 264 (1989) 12826. Further examples include GLP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹³-GLP-1(7-37) and, in particular, GLP-1(7-37)OH, VAL⁸-GLP-1(7-37), GLY⁸-GLP-1(7-37), THR⁸-GLP-1(7-37), MET⁸-GLP-1(7-37) and 4-imidazopropionyl-GLP-1. Special preference is also given to the GLP agonist analog exendin-4, described by Greig, et al., in Diabetologia, 1999, 42, 45-50.

Also included in the definition “anti-diabetic agent” are insulin sensitivity enhancers which restore impaired insulin receptor function to reduce insulin resistance and consequently enhance the insulin sensitivity. Examples include hypoglycemic thiazolidinedione derivatives (e.g., glitazone, (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297)).

Examples of anti-obesity agents that may be used in combination with the compounds of the disclosure include, but are not limited to, orlistat, sibutramine, phentermine and Cannabinoid Receptor 1 (CB1) antagonists e.g. rimonabant.

Examples of agonists of peroxisome proliferator-activator receptors that may be used in combination with the compounds of the disclosure include, but are not limited to, fenofibrate, pioglitazone, rosiglitazone, tesaglitazar, BMS-298585, L-796449, the compounds specifically described in the patent application WO 2004/103995 i.e. compounds of examples 1 to 35 or compounds specifically listed in claim 21, or the compounds specifically described in the patent application WO 03/043985 i.e. compounds of examples 1 to 7 or compounds specifically listed in claim 19 and especially (R)-1-{4-[5-methyl-2-(4-trifluoromethyl-phenyl)-oxazol-4-ylmethoxy]-benzenesulfonyl}-2,3-dihydro-1H-indole-2-carboxylic or a salt thereof.

Examples of hypolipidemic agents that may be used in combination with the compounds of the disclosure include, but are not limited to, an HMG-CoA reductase inhibitor, squalene synthase inhibitors, LXR agonist, FXR agonist, fibrates, cholesterol absorption inhibitors, nicotinic acid bile acid binding resins, bempedoic acid, nicotinic acid and other GPR109 agonists, and aspirin.

Examples of anti-hypertensive agents that may be used in combination with the compounds of the disclosure include, but are not limited to, loop diuretics; angiotensin converting enzyme (ACE); inhibitors of the Na—K-ATPase membrane pump; neutralendopeptidase (NEP) inhibitors; ACE/NEP inhibitors; angiotensin II antagonists; renin inhibitors; β-adrenergic receptor blockers; inotropic agents; calcium channel; aldosterone receptor antagonists; and aldosterone synthase inhibitors.

Examples of loop diuretics that may be used in combination with the compounds of the disclosure include, but are not limited to, ethacrynic acid, furosemide and torsemide.

The term “ACE-inhibitor” (also called angiotensin converting enzyme inhibitors) includes molecules that interrupt the enzymatic degradation of angiotensin I to angiotensin II. Such compounds may be used for the regulation of blood pressure and for the treatment of congestive heart failure. Examples include, but are not limited to, alacepril, benazepril, benazeprilat, captopril, ceronapril, cilazapril, delapril, enalapril, enaprilat, fosinopril, imidapril, lisinopril, moexipril, moveltopril, perindopril, quinapril, ramipril, spirapril, temocapril, and trandolapril, or a pharmaceutically acceptable salt thereof.

A non-limiting example of an inhibitor of the Na—K-ATPase membrane pump is digoxin.

The term “NEP inhibitor” refers to a compound that inhibits neutral endopeptidase (NEP). Examples include, but are not limited to, Candoxatril, Candoxatrilat, Dexecadotril, Ecadotril, Racecadotril, Sampatrilat, Fasidotril, Omapatrilat, Gemopatrilat, Daglutril, SCH-42495, SCH-32615, UK-447841, AVE-0848, PL-37, and (2R,4s)-5-Biphenyl-4-yl-4-(3-carboxy-propionylamino)-2-methyl-pentanoic acid ethyl ester, or a pharmaceutically acceptable salt thereof. NEP inhibitors also include Phosphono/biaryl substituted dipeptide derivatives, as disclosed in U.S. Pat. No. 5,155,100. NEP inhibitors also include N-mercaptoacyl phenylalanine derivative as disclosed in PCT application WO 2003/104200. NEP inhibitors also include dual-acting antihypertensive agents as disclosed in PCT applications WO 2008/133896, WO 2009/035543, or WO 2009/134741. Other examples include compounds disclosed in U.S. application Ser. Nos. 12/788,794; 12/788,766, and 12/947,029. NEP inhibitors also include compounds disclosed in WO 2010/136474, WO 2010/136493, WO 2011/061271, WO 2012/065953, WO 2012/065956, WO 2014/126979, and WO 2014/015965. Other examples of NEP inhibitors are compounds disclosed in WO2015116786, WO2015116760, WO2014138053, WO2014025891, WO2013184934, WO2013067163, WO2012166389, WO2012166387, WO2012112742, and WO2012082853.

The term “ACE/NEP inhibitors” refers to a compound that inhibits both angiotensin converting enzyme (ACE) and neutral endopeptidase (NEP). Examples of ACE/NEP inhibitors that may be used in combination with the compounds of the disclosure include, but are not limited to, omapatrilat, sampatrilat, and fasidotril.

The class of angiotensin II antagonists or AT₁ receptor antagonists comprises compounds having differing structural features, essentially preferred are the non-peptidic ones. Examples of angiotensin II antagonists that may be used in combination with the compounds of the disclosure include, but are not limited to, valsartan, losartan, candesartan, eprosartan, irbesartan, saprisartan, tasosartan, telmisartan, the compounds with the designation E-1477 and ZD-8731 of the following formulae

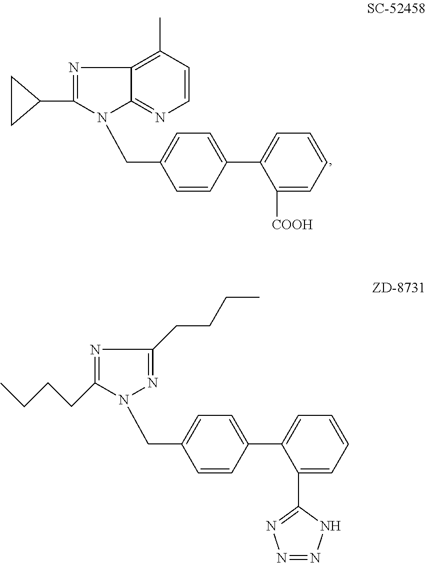 [SMILES: Cc1ccnc2c1nc(C1CC1)n2Cc1ccc(-c2ccccc2C(=O)O)cc1 ; CCCCc1nc(CCCC)n(Cc2ccc(-c3ccccc3-c3nnn[nH]3)cc2)n1]

or, in each case, a pharmaceutically acceptable salt thereof.

The term “renin inhibitor” includes ditekiren (chemical name: [1S-[1R,2R,4R(1R,2R)]]-1-[(1,1-dimethylethoxy)carbonyl]-L-proly I-L-phenylalanyl-N-[2-hydroxy-5-methyl-1-(2-methylpropyl)-4-[[[2-methyl-1-[[(2 pyridinylmrthyl)amino]carbonyl]butyl]amino]carbonyl]hexyl]-N-alfa-methyl-L-histidinamide); terlakiren (chemical name: [R—(R,S)]—N-(4-morpholinylcarbonyl)-L-phenylalanyl-N-[1-(cyclohexylmethyl)-2-hydroxy-3-(1-methylethoxy)-3-oxopropyl]-S-methyl-L-cysteineamide); Aliskiren (chemical name: (2S,4s,5S,7S)-5-amino-N-(2-carbamoyl-2,2-dimethylethyl)-4-hydroxy-7-{[4-methoxy-3-(3-methoxypropoxy)phenyl]methyl}-8-methyl-2-(propan-2-yl)nonanamide) and zankiren (chemical name: [1S-[1R[R(R)],2S,3r]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]-amino]-4-thiazolepropanamide), or, hydrochloride salts thereof, or, SPP630, SPP635 and SPP800 as developed by Speedel, or RO 66-1132 and RO 66-1168 of Formula (A) and (B):

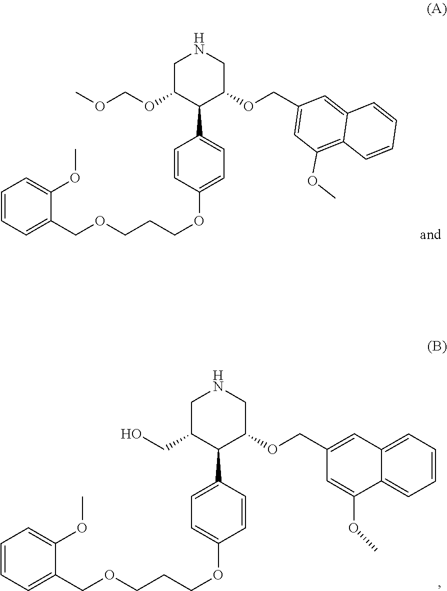

or pharmaceutically acceptable salts thereof. The term “aliskiren”, if not defined specifically, is to be understood both as the free base and as a salt thereof, especially a pharmaceutically acceptable salt thereof, most preferably a hemi-fumarate salt thereof.

Examples of β-adrenergic receptor blockers that may be used in combination with the compounds of the disclosure include, but are not limited to, acebutolol, atenolol, betaxolol, bisoprolol, metoprolol, nadolol, propranolol, sotalol, and timolol.

Examples of inotropic agents that may be used in combination with the compounds of the disclosure include, but are not limited to, digoxin, dobutamine, and milrinone; Inotropes as used herein include, for example, dobutamine, isoproterenol, milrinone, amirinone, levosimendan, epinephrine, norepinephrine, isoproterenol, and digoxin.

Examples of calcium channel blockers that may be used in combination with the compounds of the disclosure include, but are not limited to, amlodipine, bepridil, diltiazem, felodipine, nicardipine, nimodipine, nifedipine, nisoldipine and verapamil.

The class of aldosterone synthase inhibitors comprises both steroidal and non-steroidal aldosterone synthase inhibitors, the latter being most preferred. The class of aldosterone synthase inhibitors comprises compounds having differing structural features. Examples of aldosterone synthase inhibitor that can be used in combination with the compounds of the present disclosure include, but are not limited to, the (+)-enantiomer of the hydrochloride of fadrozole (U.S. Pat. Nos. 4,617,307 and 4,889,861) of formula

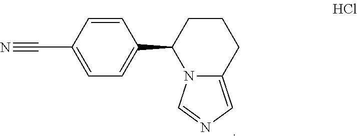 [SMILES: N#Cc1ccc([C@H]2CCCc3cncn32)cc1]

or, if appropriable, a pharmaceutically acceptable salt thereof; and compounds and analogs generically and specifically disclosed e.g. in US2007/0049616, in particular in the compound claims and the final products of the working examples, the subject-matter of the final products, the pharmaceutical preparations and the claims are hereby incorporated into the present application by reference to this publication. Examples of aldosterone synthase inhibitors that can be used in combination with the compounds of the present disclosure include, but are not limited to, without limitation 4-(6,7-dihydro-5H-pyrrolo[1,2-c]imidazol-5-yl)-3-methylbenzonitrile; 5-(2-chloro-4-cyanophenyl)-6,7-dihydro-5H-pyrrolo[1,2-c]imidazole-5-carboxylic acid (4-methoxybenzyl)methylamide; 4′-fluoro-6-(6,7,8,9-tetrahydro-5H-imidazo[1,5-a]azepin-5-yl)biphenyl-3-carbonitrile; 5-(4-Cyano-2-methoxyphenyl)-6,7-dihydro-5H-pyrrolo[1,2-c]imidazole-5-carboxylic acid butyl ester; 4-(6,7-Dihydro-5H-pyrrolo[1,2-c]imidazol-5-yl)-2-methoxybenzonitrile; 5-(2-Chloro-4-cyanophenyl)-6,7-dihydro-5H-pyrrolo[1,2-c]imidazole-5-carboxylic acid 4-fluorobenzyl ester; 5-(4-Cyano-2-trifluoromethoxyphenyl)-6,7-dihydro-5H-pyrrolo[1,2-c]imidazole-5-carboxylic acid methyl ester; 5-(4-Cyano-2-methoxyphenyl)-6,7-dihydro-5H-pyrrolo[1,2-c]imidazole-5-carboxylic acid 2-isopropoxyethyl ester; 4-(6,7-Dihydro-5H-pyrrolo[1,2-c]imidazol-5-yl)-2-methylbenzonitrile; 4-(6,7-dihydro-5H-pyrrolo[1,2-c]imidazol-5-yl)-3-fluorobenzonitrile; 4-(6,7-Dihydro-5H-pyrrolo[1,2-c]imidazol-5-yl)-2-methoxybenzonitrile; 3-Fluoro-4-(7-methylene-6,7-dihydro-5H-pyrrolo[1,2-c]imidazol-5-yl)benzonitrile; cis-3-Fluoro-4-[7-(4-fluoro-benzyl)-5,6,7,8-tetrahydro-imidazo[1,5-a]pyridin-5-yl]benzonitrile; 4′-Fluoro-6-(9-methyl-6,7,8,9-tetrahydro-5H-imidazo[1,5-a]azepin-5-yl)biphenyl-3-carbonitrile; 4′-Fluoro-6-(9-methyl-6,7,8,9-tetrahydro-5H-imidazo[1,5-a]azepin-5-yl)biphenyl-3-carbonitrile or in each case, the (R) or (S) enantiomer thereof; or if appropriable, a pharmaceutically acceptable salt thereof.

The term aldosterone synthase inhibitors also include, but are not limited to, compounds and analogs disclosed in WO2008/076860, WO2008/076336, WO2008/076862, WO2008/027284, WO2004/046145, WO2004/014914, and WO2001/076574.

Furthermore, Aldosterone synthase inhibitors also include, but are not limited to, compounds and analogs disclosed in U.S. patent applications US2007/0225232, US2007/0208035, US2008/0318978, US2008/0076794, US2009/0012068, US20090048241 and in PCT applications WO2006/005726, WO2006/128853, WO2006128851, WO2006/128852, WO2007065942, WO2007/116099, WO2007/116908, WO2008/119744 and in European patent application EP 1886695. Preferred aldosterone synthase inhibitors suitable for use in the present disclosure include, without limitation 8-(4-Fluorophenyl)-5,6-dihydro-8H-imidazo[5,1-c1][1,4]oxazine; 4-(5,6-Dihydro-8H-imidazo[5,1-c][1,4]oxazin-8-yl)-2-fluorobenzonitrile; 4-(5,6-Dihydro-8H-imidazo[5,1-c][1,4]oxazin-8-yl)-2,6-difluorobenzonitrile; 4-(5,6-Dihydro-8H-imidazo[5,1-c][1,4]oxazin-8-yl)-2-methoxybenzonitrile; 3-(5,6-Dihydro-8H-imidazo[5,1-c][1,4]oxazin-8-yl)benzonitrile; 4-(5,6-Dihydro-8H-imidazo[5,1-c][1,4]oxazin-8-yl)phthalonitrile; 4-(8-(4-Cyanophenyl)-5,6-dihydro-8H-imidazo[5,1-c][1,4]oxazin-8-yl)benzonitrile; 4-(5,6-Dihydro-8H-imidazo[5,1-c][1,4]oxazin-8-yl)benzonitrile; 4-(5,6-Dihydro-8H-imidazo[5,1-c][1,4]oxazin-8-yl)naphthalene-1-carbonitrile; 8-[4-(1H-Tetrazol-5-yl)phenyl]-5,6-dihydro-8H-imidazo[5,1-c][1,4]oxazine as developed by Speedel or in each case, the (R) or (S) enantiomer thereof; or if appropriable, a pharmaceutically acceptable salt thereof.

Aldosterone synthase inhibitors useful in said combination include, but are not limited to, compounds and analogs generically and specifically disclosed e.g. in WO 2009/156462 and WO 2010/130796, in particular in the compound claims and the final products of the working examples, the subject-matter of the final products, the pharmaceutical preparations and the claims. Preferred Aldosterone Synthase inhibitors suitable for combination in the present disclosure include, 3-(6-Fluoro-3-methyl-2-pyridin-3-yl-1H-indol-1-ylmethyl)-benzonitrile hydrochloride, 1-(4-Methanesulfonyl-benzyl)-3-methyl-2-pyridin-3-yl-1H-indole, 2-(5-Benzyloxy-pyridin-3-yl)-6-chloro-1-methyl-1H-indole, 5-(3-Cyano-1-methyl-1H-indol-2-yl)-nicotinic acid ethyl ester, N-[5-(6-chloro-3-cyano-1-methyl-1H-indol-2-yl)-pyridin-3-ylmethyl]-ethanesulfonamide, Pyrrolidine-1-sulfonic acid 5-(6-chloro-3-cyano-1-methyl-1H-indol-2-yl)-pyridin-3-yl ester, N-Methyl-N-[5-(1-methyl-1H-indol-2-yl)-pyridin-3-ylmethyl]-methanesulfonamide, 6-Chloro-1-methyl-2-{5-[(2-pyrrolidin-1-yl-ethylamino)-methyl]-pyridin-3-yl}-1H-indole-3-carbonitrile, 6-Chloro-2-[5-(4-methanesulfonyl-piperazin-1-ylmethyl)-pyridin-3-yl]-1-methyl-1H-indole-3-carbonitrile, 6-Chloro-1-methyl-2-{5-[(1-methyl-piperidin-4-ylamino)-methyl]-pyridin-3-yl}-1H-indole-3-carbonitrile, Morpholine-4-carboxylic acid [5-(6-chloro-3-cyano-1-methyl-1H-indol-2-yl)-pyridin-3-ylmethyl]-amide, N-[5-(6-Chloro-1-methyl-1H-indol-2-yl)-pyridin-3-ylmethyl]-ethanesulfonamide, C,C,C-Trifluoro-N-[5-(1-methyl-1H-indol-2-yl)-pyridin-3-ylmethyl]-methanesulfonamide, N-[5-(3-Chloro-4-cyano-phenyl)-pyridin-3-yl]-4-trifluoromethyl-benzenesulfonamide, N-[5-(3-Chloro-4-cyano-phenyl)-pyridin-3-yl]-1-phenyl-methanesulfonamide, N-(5-(3-chloro-4-cyanophenyl)pyridin-3-yl)butane-1-sulfonamide, N-(1-(5-(4-cyano-3-methoxyphenyl)pyridin-3-yl)ethyl)ethanesulfonamide, N-((5-(3-chloro-4-cyanophenyl)pyridin-3-yl)(cyclopropyl)methyl)ethanesulfonamide, N-(cyclopropyl(5-(1H-indol-5-yl)pyridin-3-yl)methyl)ethanesulfonamide, N-(cyclopropyl(5-naphtalen-1-yl-pyridin-3-yl)methyl)ethanesulfonamide, Ethanesulfonic acid [5-(6-chloro-1-methyl-1H-pyrrolo[2,3-b]pyridin-2-yl)-pyridin-3-ylmethyl]-amide and Ethanesulfonic acid {[5-(3-chloro-4-cyano-phenyl)-pyridin-3-yl]-cyclopropyl-methyl}-ethyl-amide.

Lipid-lowering agents are known in the art, and described, e.g., in Goodman and Gilman's The Pharmacological Basis of Therapeutics, 11th Ed., Brunton, Lazo and Parker, Eds., McGraw-Hill (2006); 2009 Physicians' Desk Reference (PDR), for example, in the 63rd (2008) Eds., Thomson PDR.

“Combination therapy” is intended to embrace administration of these therapeutic agents in a sequential manner, wherein each therapeutic agent is administered at a different time and in any order, or in alternation and in any order, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

In accordance with the foregoing, the present disclosure also provides a therapeutic combination, e.g., a kit, kit of parts, e.g., for use in any method as defined herein, comprising a compound of Formula (I), or a pharmaceutically acceptable salt thereof, to be used concomitantly or in sequence with at least one pharmaceutical composition comprising at least another therapeutic agent, selected from a hypolipidemic agent, niacin or analogs thereof, a bile acid sequestrant, a thyroid hormone mimetic, a thyroid hormone receptor (THR) β-selective agonist, a microsomal triglyceride transfer protein (MTP) inhibitor, an acyl CoA:diacylglycerol acyltransferase (DGAT) inhibitor, a Niemann Pick C1-like 1 (NPC1-L1) inhibitor, an agonist of ATP Binding Cassette (ABC) proteins G5 or G8, an inhibitory nucleic acid targeting PCSK9, an inhibitory nucleic acid targeting apoB100, apoA-I up-regulator/inducer, an ABCA1 stabilizer or inducer, phospholipid transfer protein (PLTP) inhibitor, fish oil, an antidiabetic agent, an anti-obesity agent, an agonist of peroxisome proliferator-activator receptors, ATP citrate lyase (ACL) inhibitor, and an anti-hypertensive agent, or a pharmaceutically acceptable salt thereof. The kit may comprise instructions for its administration. The combination can be a fixed combination (e.g. in the same pharmaceutical composition) or a free combination (e.g. in separate pharmaceutical compositions).

Similarly, the present disclosure provides a kit of parts comprising: (i) a pharmaceutical composition of the disclosure; and (ii) a pharmaceutical composition comprising a compound selected from a hypolipidemic agent, niacin or analogs thereof, a bile acid sequestrant, a thyroid hormone mimetic, a thyroid hormone receptor (THR) β-selective agonist, a microsomal triglyceride transfer protein (MTP) inhibitor, an acyl CoA:diacylglycerol acyltransferase (DGAT) inhibitor, a Niemann Pick C1-like 1 (NPC1-L1) inhibitor, an agonist of ATP Binding Cassette (ABC) proteins G5 or G8, an inhibitory nucleic acid targeting PCSK9, an inhibitory nucleic acid targeting apoB100, apoA-I up-regulator/inducer, an ABCA1 stabilizer or inducer, phospholipid transfer protein (PLTP) inhibitor, fish oil, an antidiabetic agent, an anti-obesity agent, an agonist of peroxisome proliferator-activator receptors, ATP citrate lyase (ACL) inhibitor, and an anti-hypertensive agent, or a pharmaceutically acceptable salt thereof, in the form of two separate units of the components (i) to (ii).

Likewise, the present disclosure provides a method as defined above comprising co-administration, e.g., concomitantly or in sequence, of a therapeutically effective amount of a compound of Formula (I), or a pharmaceutically acceptable salt thereof, and a second drug substance, said second drug substance being a hypolipidemic agent, niacin or analogs thereof, a bile acid sequestrant, a thyroid hormone mimetic, a thyroid hormone receptor (THR) β-selective agonist, a microsomal triglyceride transfer protein (MTP) inhibitor, an acyl CoA:diacylglycerol acyltransferase (DGAT) inhibitor, a Niemann Pick C1-like 1 (NPC1-L1) inhibitor, an agonist of ATP Binding Cassette (ABC) proteins G5 or G8, an inhibitory nucleic acid targeting PCSK9, an inhibitory nucleic acid targeting apoB100, apoA-I up-regulator/inducer, an ABCA1 stabilizer or inducer, phospholipid transfer protein (PLTP) inhibitor, fish oil, an antidiabetic agent, an anti-obesity agent, an agonist of peroxisome proliferator-activator receptors, ATP citrate lyase (ACL) inhibitor, and an anti-hypertensive agent, e.g., as indicated above.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof, which may suggest themselves to those, skilled in the art without departing from the spirit of the present disclosure and/or scope of the appended claims.

### Analytical Methods, Materials, and Instrumentation

Unless otherwise noted, reagents and solvents were used as received from commercial suppliers. Proton nuclear magnetic resonance (NMR) spectra were obtained with a Varian spectrometer at 400 MHz, a Bruker spectrometer at 300 MHz or 400 MHz. Spectra are given in ppm (δ) and coupling constants, J, are reported in Hertz. Tetramethylsilane (TMS) or the solvent peak was used as an internal standard.

Abbreviations

| | |
|---|---|
| ACN | Acetonitrile |
| AcOEt | Ethyl acetate |
| AcOH | Acetic acid |
| AcOtBu | tert-Butyl acetate |
| Arg | Arginine |
| BME | β-Mercaptoethanol |
| Boc | tert-Butyloxycarbonyl |
| Boc₂O | Di-tert-butyl-dicarbonate |
| DBU | 1,8-Diazabicyclo[5.4.0]undec-7-ene |
| DCE | 1,2-Dichloroethane |
| DCM | Dichloromethane |
| DEAD | Diethylazodicarboxylate |
| DIAD | Diisopropylazodicarboxylate |
| DIC | N,N′-Diisopropylcarbodiimide |
| DIEA, DIPEA | N,N-Diisopropylethylamine |
| DMA | N,N-Dimethylacetamide |
| DMAP | 4-Dimethylaminopyridine |
| DME | 1,2-Dimethoxyethane |
| DMF | N,N-Dimethylformamide |
| DMSO | Dimethylsulfoxide |
| DMT-MM | 4-(4,6-Dimethoxy-1,3,5- |
| | triazin-2-yl)-4- |
| | methylmorpholinium chloride |
| DPPA | Diphenylphosphorylazide |
| EDC | N-(3-Dimethylaminopropyl)-N- |
| | ethylcarbodiimide HCl |
| EtOAc or EA | Ethyl acetate |
| EtOH | Ethanol |
| Fmoc | 9-Fluorenylmethyloxycarbonyl |
| FmocOSu | 9-Fluorenylmethyl-N- |
| | hydroxysuccinimide |
| GC | Gas chromatography |
| HATU | 1-[Bis(dimethylamino)methylene]- |
| | 1H-1,2,3-triazolo[4,5- |
| | b]pyridinium 3-oxid |
| | hexafluorophosphate |
| HCTU | O-(6-Chlorobenzotriazol-1-yl)- |
| | N,N,N′,N′-tetramethyluronium |
| | hexafluorophosphate |
| HFIP | 1,1,1,3,3,3- |
| | Hexafluoropropan-2-ol |
| HOAt | 1-Hydroxy-7- |
| | azabenzotriazole |
| HOBt | 1-Hydroxybenzotriazole |
| HOSu | N-Hydroxysuccinimide |
| IPA, i-PrOH | iso-propanol |
| KHMDS | Potassium |
| | hexamethyldisilazane |
| KN(TMS)₂ | Potassium |
| | hexamethyldisilazane |
| KOtBu, | Potassium tert-butylate |
| tBuOK | |
| LCMS | Liquid chromatography mass |
| | spectrometry |
| LiHMDS | Lithium hexamethyldisilazane |
| mCPBA | meta-Chloroperbenzoic acid |
| MeI | Methyl iodide |
| MeOH | Methanol |
| Me₃SiCl, | trimethylsilyl chloride |
| TMSCI | |
| MMPP | Magnesium |
| | monoperoxyphthalate |
| NaBH(OAc)₃ | Sodium triacetoxyborohydride |
| NaOAc | Sodium acetate |
| NaOMe | Sodium methanolate |
| NBS | N-Bromosuccinimide |
| nBuOH | n-Butanol |
| NIS | N-Iodosuccinimide |
| NMP | N-Methylpyrrolidine |
| Ns | 4-Nosyl |
| o/n | overnight |
| P1-tBu | N′-tert.-Butyl- |
| | N,N,N′,N′,N″,N″- |
| | hexamethylphosphorimidictriamide |
| PE | Petroleum ether |
| Pmc | 2,2,5,7,8- |
| | Pentamethylchroman-6- |
| | sulfonyl |
| PPHF | Pyridinium poly(hydrogen |
| | fluoride) |
| PS | Polystyrene |
| PTSA | para-toluenesulfonic acid |
| PyOxim | (((1-cyano-2-ethoxy-2- |
| | oxoethylidene)amino)oxy)tri(pyrrolidin- |
| | 1-yl)phosphonium |
| | hexafluorophosphate(V) |
| Rt | Retention time |
| Ser | Serine |
| TBAF | Tetrabutylammonium fluoride |
| TBME | tert-butyl methyl ether |
| TBS | tert-butyldimethylsilyl |
| TBTA | Tris[(1-benzyl-1H-1,2,3- |
| | triazol-4-yl)methyl]amine |
| TBTU | 2-(1 H-Benzotriazol-1-yl)- |
| | 1,1,3,3-tetramethyluronium- |
| | tetrafluoroborate |
| TEA | Triethylamine |
| TFA | Trifluoroacetic acid |
| TFFH | Tetramethylfluoroformamidinium |
| | hexafluorophosphate |
| THF | Tetrahydrofuran |
| TMOF | Trimethylorthoformate |
| TMSOTf | Trimethylsilyltriflate |
| TosBIC | alpha-Tosylbenzyl isocyanide |
| TOSMIC | Toluenesulfonylmethyl |
| | isocyanide |
| t_{R} | Retention time |
| TTPA | Tris(N,N- |
| | tetramethylene)phosphoric |
| | acid triamide |

### General Preparative HPLC Purification Procedure and Mass Spectra

The final products were purified by preparative reversed-phase HPLC and reversed-phase flash chromatography using a Waters XBridge Prep C18 OBD Column, 5 μm, 30 mm×250 mm; a Waters XBridge OBD Prep Column Reversed-Phase BEH Column, 5 um, 19×150 mm; a Waters XSelect CSH C18 OBD Prep Column, 5 μm, 19 mm×250 mm; or a 50 gram, a 100 gram or a 275 gram RediSep Rf Gold C18 High Performance Column.

The following mobile phases were used:
Eluent A: 0.1% TFA in H₂O and eluent B: ACN
Eluent A: 0.5% HCOOH in H₂O and eluent B: ACN
Eluent A: 0.01 M HCl in H₂O and eluent B: ACN
Eluent A: 0.01 M NH₄OH in H₂O and eluent B: ACN

Gradients were designed based on the specific requirements of the separation problem. Pure products were lyophilized from ACN/H₂O and obtained, depending on the used eluents, as a free base or the corresponding trifluoroacetate, formate or hydrochloride salt. In several cases, the salt form was changed using the following methods:
The TFA salt was partitioned between EtOAc and 5% aq. NaHCO₃. The organic layer was washed with 5% aq. NaHCO₃ (2×) and brine, dried over Na₂SO₄, filtered and evaporated to dryness in vacuo. The residue was dissolved in ACN/H₂O (1:1) and 1 M HCl (˜1.5-3 eq per basic center), then lyophilized to afford the hydrochloride of the product as a white solid.
The formate salt or trifluoroacetate was dissolved in EtOAc and washed with saturated aqueous sodium bicarbonate. The aqueous layer was then extracted with fresh EtOAc (×2). The combined organics were dried over sodium sulfate, filtered, and concentrated in vacuo to yield a white solid as the free base.

### LCMS Analytical Methods:

The products were analyzed by the analytical methods described below.

| | | |
|---|---|---|
| Time | Eluent A [%] | Eluent B [%] |

Analytical method 1
Agilent 1100/1200 ALS system/Waters ZQD
MS system
Eluent A: 0.05% Trifluoroacetic acid in H₂O
Eluent B: Acetonitrile
Column temperature: 40° C.
Flow: 2.0 mL/min
Column: SunFire C18, 3.5 μm, 3.0 × 30 mm
Gradient:

| | | |
|---|---|---|
| 0.00 | 95 | 5 |
| 1.70 | 5 | 95 |
| 2.00 | 5 | 95 |
| 2.10 | 95 | 5 |

Analytical method 2
Waters Acquity UPLC system/Waters SQD
MS system
Eluent A: 5 mM Ammonium hydroxide in H₂O
Eluent B: 5 mM Ammonium hydroxide in
acetonitrile
Column temperature: 50° C.
Flow: 1.0 mL/min
Column: Acquity UPLC BEH C18, 1.7 μm, 2.1 × 50 mm
Gradient:

| | | |
|---|---|---|
| 0.00 | 98 | 2 |
| 4.40 | 2 | 98 |
| 5.15 | 2 | 98 |
| 5.19 | 98 | 2 |

Analytical method 3
Waters Acquity UPLC system/Waters Xevo
G2 Qtof MS system
Eluent A: 0.1% Formic acid in H₂O
Eluent B: 0.1% Formic acid in acetonitrile
Column temperature: 50° C.
Flow: 1.0 mL/min
Column: Acquity UPLC BEH C18, 1.7 μm, 2.1 × 50 mm
Gradient:

| | | |
|---|---|---|
| 0.00 | 98 | 2 |
| 0.06 | 98 | 2 |
| 1.76 | 2 | 98 |
| 2.00 | 2 | 98 |
| 2.16 | 98 | 2 |

Analytical method 4
Waters Acquity UPLC system/Waters SQD
MS system
Eluent A: 0.1% Formic acid in H₂O
Eluent B: 0.1% Formic acid in acetonitrile
Column temperature: 50° C.
Flow: 1.0 mL/min
Column: Acquity UPLC BEH C18, 1.7 μm, 2.1 × 50 mm
Gradient:

| | | |
|---|---|---|
| 0.00 | 98 | 2 |
| 4.40 | 2 | 98 |
| 5.15 | 2 | 98 |
| 5.19 | 98 | 2 |

Analytical method 5
Waters Acquity UPLC system/Waters SQD
MS system
Eluent A: 5 mM Ammonium hydroxide in H₂O
Eluent B: 5 mM Ammonium hydroxide in
acetonitrile
Column temperature: 50° C.
Flow: 1.0 mL/min
Column: Acquity UPLC BEH C18, 1.7 μm, 2.1 × 30 mm
Gradient:

| | | |
|---|---|---|
| 0.00 | 98 | 2 |
| 0.10 | 98 | 2 |
| 1.50 | 2 | 98 |
| 1.80 | 2 | 98 |
| 1.90 | 98 | 2 |
| 2.00 | 98 | 2 |

Analytical method 6
Waters Acquity UPLC system/Waters Xevo
G2 Qtof MS system
Eluent A: 0.1% Formic acid in H₂O
Eluent B: 0.1% Formic acid in acetonitrile
Column temperature: 50° C.
Flow: 1.0 mL/min
Column: Acquity UPLC BEH C18, 1.7 μm, 2.1 × 50 mm
Gradient:

| | | |
|---|---|---|
| 0.00 | 98 | 2 |
| 4.40 | 2 | 98 |
| 5.15 | 2 | 98 |
| 5.19 | 98 | 2 |

Analytical method 7
Waters Acquity UPLC system/Waters SQD
MS system
Eluent A: 0.1% Formic acid in H₂O
Eluent B: 0.1% Formic acid in acetonitrile
Column temperature: 50° C.
Flow: 1.0 mL/min
Column: Acquity UPLC BEH C18, 1.7 μm, 2.1 × 30 mm
Gradient:

| | | |
|---|---|---|
| 0.00 | 98 | 2 |
| 0.10 | 98 | 2 |
| 1.50 | 2 | 98 |
| 1.80 | 2 | 98 |
| 1.90 | 98 | 2 |
| 2.00 | 98 | 2 |

Analytical method 8
Agilent 1100/1200 ALS system/Waters ZQD
MS system
Eluent A: 5 mM Ammonium hydroxide in H₂O
Eluent B: Acetonitrile
Column temperature: 40° C.
Flow: 2.0 mL/min
Column: XBridge C18, 3.5 μm, 3.0 × 30 mm
Gradient:

| | | |
|---|---|---|
| 0.00 | 95 | 5 |
| 1.70 | 5 | 95 |
| 2.00 | 5 | 95 |
| 2.10 | 95 | 5 |

Analytical method 9
Waters Acquity UPLC/Waters QTof MS
system
Eluent A: 0.05% Trifluoroacetic acid in H₂O
Eluent B: 0.04% Trifluoroacetic acid in
acetonitrile
Column temperature: 80° C.
Flow: 0.5 mL/min
Column: Acquity UPLC CSH C18, 1.7 μm, 2.1 mm × 100 mm
Gradient:

| | | |
|---|---|---|
| 0.00 | 95 | 5 |
| 0.20 | 95 | 5 |
| 9.40 | 2 | 98 |
| 9.80 | 2 | 98 |
| 9.90 | 95 | 5 |
| 10.00 | 95 | 5 |

Analytical method 10
Waters Acquity UPLC/SQD MS system
Eluent A: 0.05% Formic acid and 3.75 mM
ammonium acetate in H₂O
Eluent B: 0.04% Formic acid in acetonitrile
Column temperature: 60° C.
Flow: 1.0 mL/min
Column: Acquity UPLC HSS T3, 1.8 μm, 2.1 mm × 50 mm
Gradient:

| | | |
|---|---|---|
| 0.00 | 95 | 5 |
| 1.40 | 2 | 98 |
| 1.80 | 2 | 98 |
| 1.90 | 95 | 5 |
| 2.00 | 95 | 5 |

Analytical method 11
Waters Acquity UPLC/SQD MS system
Eluent A: 0.05% Formic acid and 3.75 mM
ammonium acetate in H₂O
Eluent B: 0.04% Formic acid in acetonitrile
Column temperature: 60° C.
Flow: 1.0 mL/min
Column: Acquity UPLC HSS T3, 1.8 μm, 2.1 mm × 50 mm
Gradient:

| | | |
|---|---|---|
| 0.00 | 99 | 1 |
| 1.40 | 2 | 98 |
| 1.80 | 2 | 98 |
| 1.90 | 99 | 1 |
| 2.00 | 99 | 1 |

Analytical method 12
Waters Acquity UPLC/SQD MS system
Eluent A: 0.05% Formic acid and 3.75 mM
ammonium acetate in H₂O
Eluent B: 0.04% Formic acid in acetonitrile
Column temperature: 60° C.
Flow: 1.0 mL/min
Column: Acquity UPLC HSS T3, 1.8 μm, 2.1 mm × 50 mm
Gradient:

| | | |
|---|---|---|
| 0.00 | 95 | 5 |
| 9.40 | 2 | 98 |
| 9.80 | 2 | 98 |
| 9.90 | 95 | 5 |
| 10.00 | 95 | 5 |

Analytical method 13
Waters Acquity UPLC/Waters QTof MS
system
Eluent A: 0.05% Trifluoroacetic acid in H₂O
Eluent B: 0.04% Trifluoroacetic acid in
acetonitrile
Column temperature: 80° C.
Flow: 0.8 mL/min
Column: Acquity UPLC CSH C18, 1.7 μm, 2.1 mm × 100 mm
Gradient:

| | | |
|---|---|---|
| 0.00 | 95 | 5 |
| 0.20 | 95 | 5 |
| 9.40 | 2 | 98 |
| 9.80 | 2 | 98 |
| 9.90 | 95 | 5 |
| 10.00 | 95 | 5 |

Analytical method 14
Waters Acquity UPLC/Waters QTof MS
system
Eluent A: 0.05% Trifluoroacetic acid in H₂O
Eluent B: 0.04% Trifluoroacetic acid in
acetonitrile
Column temperature: 80° C.
Flow: 0.5 mL/min
Column: Acquity UPLC CSH C18, 1.7 μm, 2.1 mm × 100 mm
Gradient:

| | | |
|---|---|---|
| 0.00 | 95 | 5 |
| 0.20 | 95 | 5 |
| 9.40 | 2 | 98 |
| 10.40 | 2 | 98 |
| 10.50 | 95 | 5 |
| 11.00 | 95 | 5 |

Analytical method 15
Waters Acquity UPLC/Waters QTof MS
system
Eluent A: 0.05% Trifluoroacetic acid in H₂O
Eluent B: 0.04% Trifluoroacetic acid in
acetonitrile
Column temperature: 80° C.
Flow: 0.8 mL/min
Column: Acquity UPLC BEH C18, 300 Å, 1.7 μm, 2.1 × 50 mm
Gradient:

| | | |
|---|---|---|
| 0.0 | 95 | 5 |
| 0.2 | 95 | 5 |
| 4.4 | 2 | 98 |
| 4.8 | 2 | 98 |
| 4.9 | 95 | 5 |
| 5.0 | 95 | 5 |

Analytical method 16
Waters Acquity UPLC/Waters QDa MS
system
Eluent A: 5 mM Ammonium hydroxide in H₂O
Eluent B: 5 mM Ammonium hydroxide in
acetonitrile
Column temperature: 50° C.
Flow: 1.0 mL/min
Column: Acquity UPLC BEH C18, 1.7 μm, 2.1 × 30 mm
Gradient:

| | | |
|---|---|---|
| 0.00 | 99 | 1 |
| 1.20 | 70 | 30 |
| 2.15 | 2 | 98 |
| 2.19 | 99 | 1 |

### Example 1: General Synthesis Procedure for Assembly of Pentamer Compounds

The cyclic pentamer compounds were assembled on solid phase and in solution from blocks A (succinate), B (diamine), C (β3-amino acid), D (α-amino acid (α-aa)), and F (aldehyde) using Methods A-H. The building blocks used for the syntheses are summarized herein below. For the solid phase strategies, PS-2-Chlorotrityl chloride resin was used.

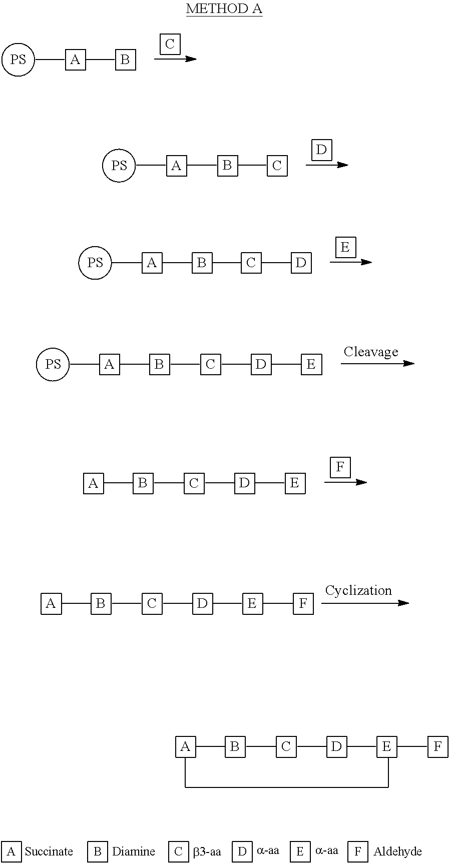

### Method A:

The monomer building blocks C, D, and E were coupled sequentially to the polymer bound dimer intermediate A-B using standard Fmoc-chemistry. Coupling of PS-A-B with α-amino acid C using standard coupling conditions (e.g., an amide coupling reagent in a solvent followed by removal of the Fmoc protecting group under basic conditions) provided PS-A-B-C. The deprotection and coupling steps were repeated followed by cleavage from the resin (e.g., by treatment with HFIP) to provide A-B-C-D-E. Reductive alkylation with aldehyde F using a reducing agent in a solvent provided intermediate pentamer A-B-C-D-E. Cyclization using standard coupling conditions using an amide coupling reagent followed by deprotection provided the desired cyclic pentamer compound of Formula (I).

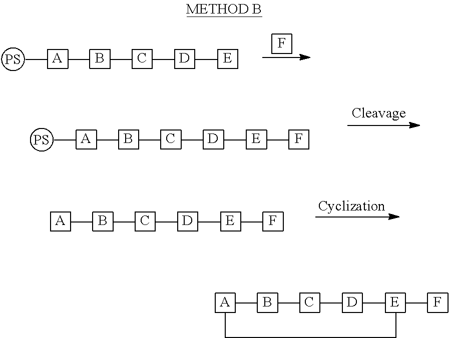

Reductive alkylation with aldehyde F was done on the polymer-bound pentamer compound. After cleavage from the resin, the cyclization was carried out as described in Method A to provide the desired compound of Formula (I).

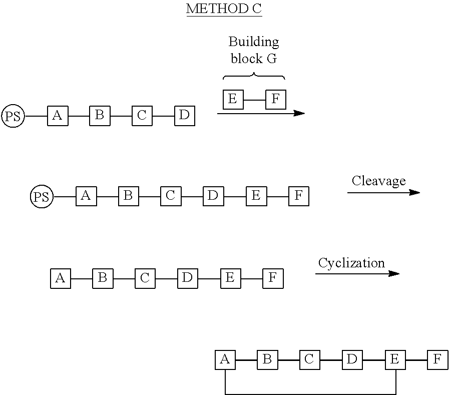

A preformed building block G composed of the building blocks E and F was coupled to the polymer bound tetramer. Cleavage from the resin and cyclization were carried out as described in Method A to provide the desired compound of Formula (I).

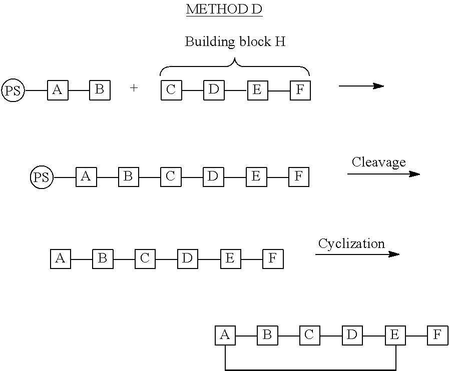

A preformed building block H composed of the building blocks C, D, E and F was coupled to the polymer bound dimer A-B. The synthesis of building block H starting from the building blocks C, D, E and F is described herein below in Section 0. Cleavage from the resin and cyclization were carried out as described in Method A to provide the desired compound of Formula (I).

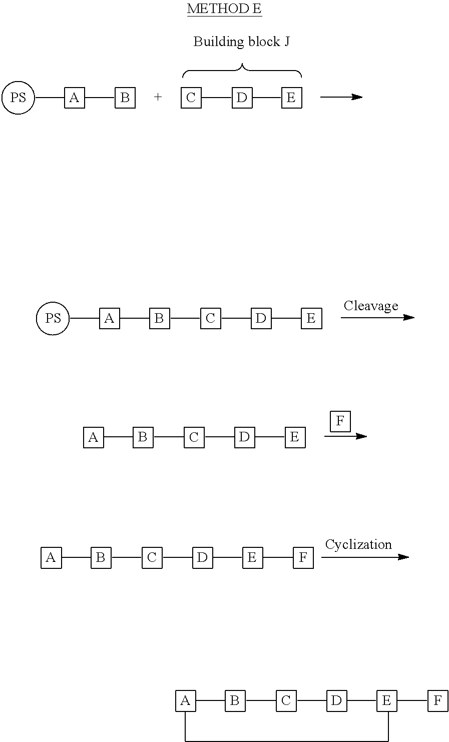

A preformed building block J composed of the building blocks C, D, and E was coupled to the polymer bound dimer A-B. The synthesis of building block J starting from the building blocks C, D and E is described in Section 0. Cleavage from the resin, reductive alkylation with Aldehyde F and cyclization were carried out as described in Method A to provide the desired compound of Formula (I).

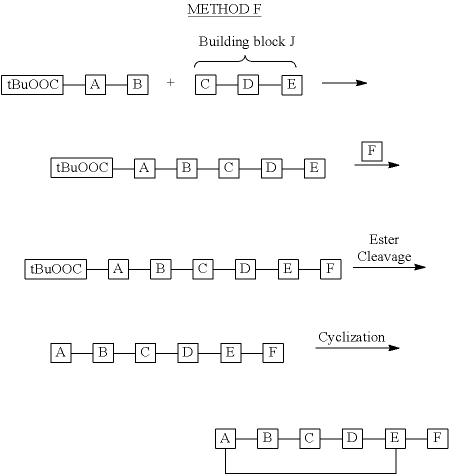

The dimer AB protected as a tBu-ester was coupled with the trimer building block J. After reductive alkylation with aldehyde F, the tBu-ester was cleaved using acidic deprotection conditions. Cyclization was carried out as described in Method A to provide the desired compound of Formula (I).

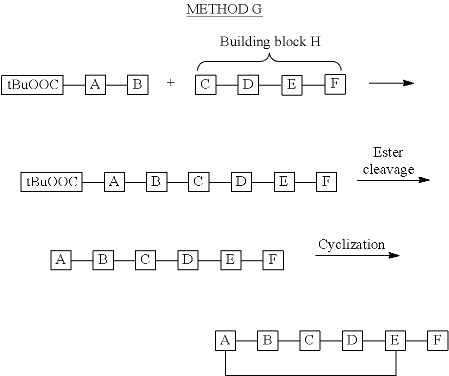

Building H was coupled to the C-terminal of tBu protected dimer A-B. Acidic cleavage of the ester followed by cyclization as described in Method A provided the desired compound of Formula (I).

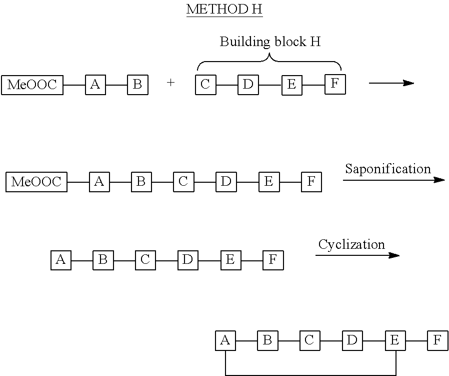

Building H was coupled to the C-terminal of methyl protected dimer A-B. Saponification of the ester followed by cyclization as described in Method A provided the desired compound of Formula (I).

In Methods A-H, a variety of coupling reagents were used for amide formation including assembly of the linear peptide as well as cyclization, e.g. HATU, PyOxim, TBTU, DMT-MM. For reductive alkylation with building block F, various reducing agents were used (e.g. NaBH(OAc)₃, NaCNBH₃ and NaBH₄). In some cases, NaCNBH₃ led to the formation of boron complexes that required treatment with Pd/C or PtO₂ for hydrolysis.

### Example 2.1: Method A—Synthesis of (2S,5S,8S,13S,16R)-16-benzyl-8-(4-chlorobenzyl)-1-(4-ethyl-2-(4-(1-methyl-2-(pyrrolidin-1-ylmethyl)-1H-imidazol-5-yl)phenoxy)benzyl)-5-(methoxymethyl)-2,7,13,14-tetramethyl-1,4,7,11,14-pentaazacyclooctadecane-3,6,10,15,18-pentaone hydrochloride (Compound 150)

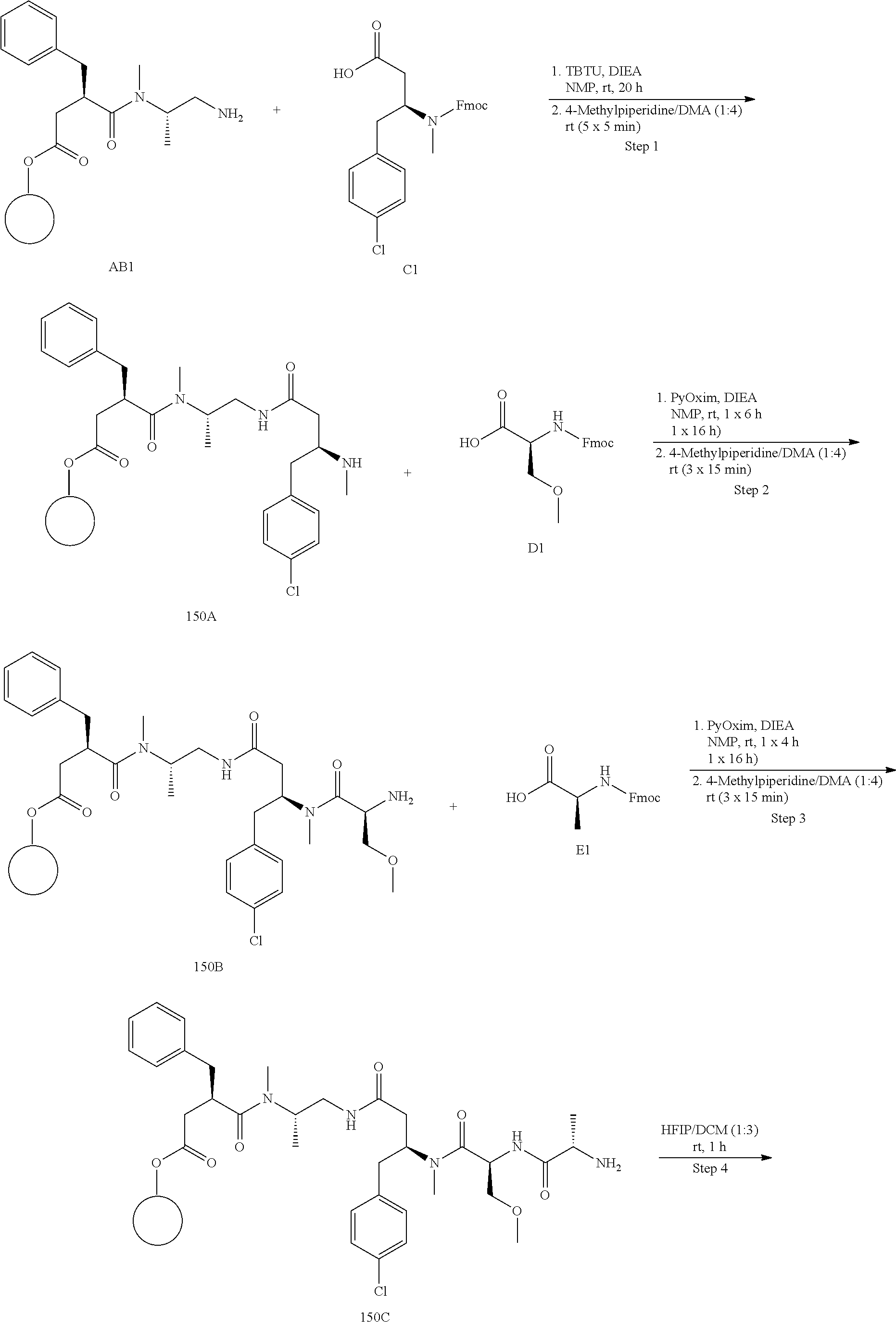

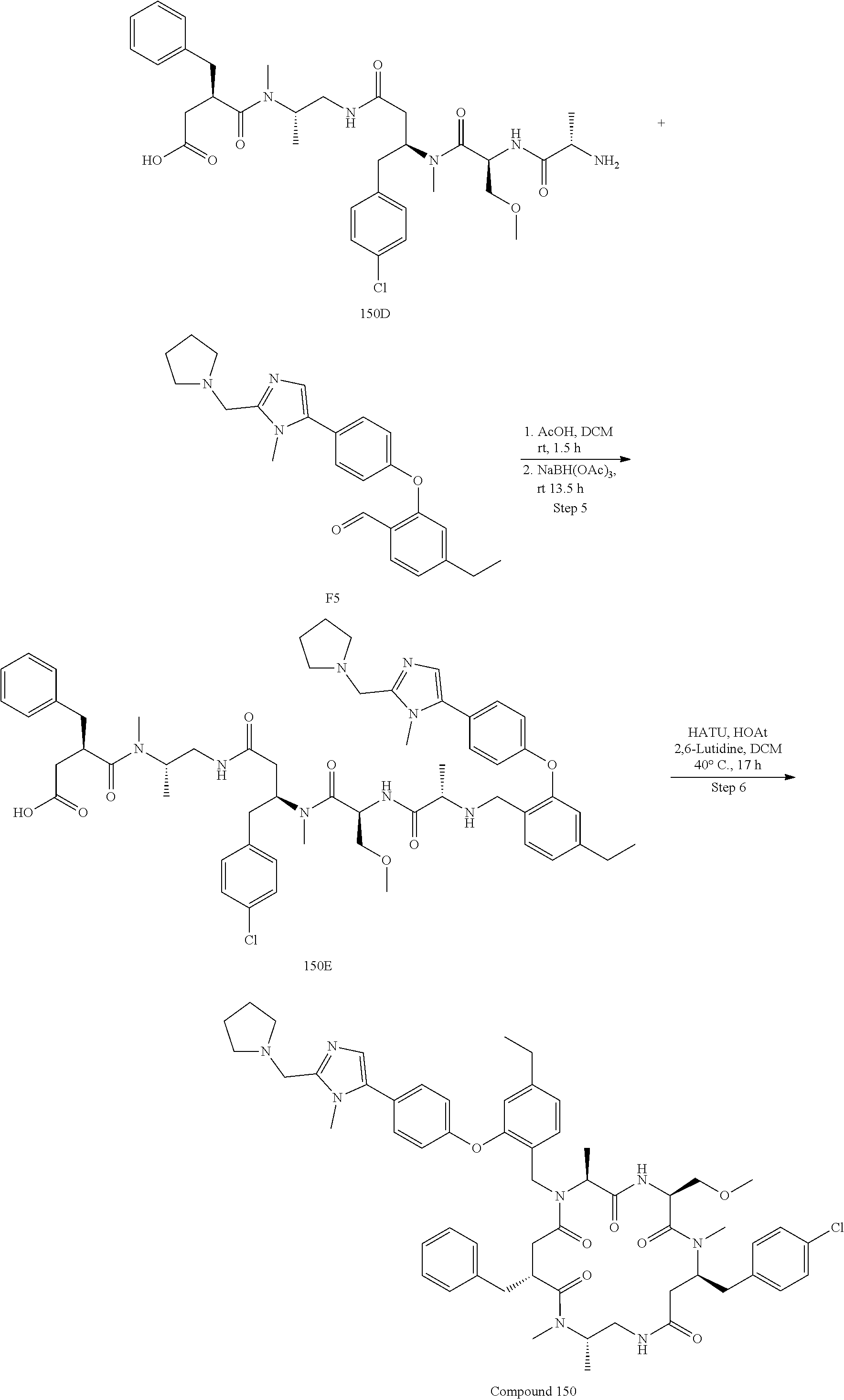

### Step 1. PS-(2-Chlorotrityl) (R)-3-benzyl-4-(((S)-1-((S)-4-(4-chlorophenyl)-3-(methylamino)butanamido)-propan-2-yl)(methyl)amino)-4-oxobutanoate (150A)

Step 1-1. A solution of C1 (4.05 g, 9.00 mmol), TBTU (2.89 g, 9.00 mmol) and DIEA (1.729 mL, 9.90 mmol) in NMP (70 mL) was shaken for 2 min at rt, and was then added to AB1 (6.00 mmol) that had been washed with NMP (3×). The resulting suspension was shaken for 20 h at rt, then filtered and the resin was washed with DMA (3×). For capping, Ac₂O/pyridine/DMA (1:1:8) (70 mL) was added and the resulting suspension was shaken for 15 min at rt. The resin was drained, and then washed with DMA (3×).

Step 1-2. Fmoc-deprotection was done by repetitive treatment with 4-methylpiperidine/DMA (1:4) (5×70 mL, each time shaking for 5 min at rt). The cleavage solutions were collected and used to determine the loading of the resin via UV spectra. After Fmoc removal, the resin was washed with DMA (3×), DCM (3×), DMA (3×) and DCM (5×) and dried in vacuo to provide 150A (11.58 g; 5.532 mmol; loading: 0.478 mmol/g).

### Step 2. PS-(2-Chlorotrityl) (4S,7S,12S,15R)-4-amino-15-benzyl-7-(4-chlorobenzyl)-6,12,13-trimethyl-5,9,14-trioxo-2-oxa-6,10,13-triazaheptadecan-17-oate (150B)

Step 2-1. A solution of Fmoc-O-methyl-L-serine (D1, 2.292 g, 6.72 mmol), PyOxim (3.54 g, 6.72 mmol), and DIEA (2.346 mL, 13.43 mmol) in NMP (55 mL) was shaken for 2 min at rt, and was then added to 150A (4.477 mmol) that had been washed with NMP (3×). The resulting suspension was shaken for 6 h at rt, and then filtered. A solution of Fmoc-O-methyl-L-serine (D1) (1.528 g, 4.48 mmol), PyOxim (2.361 g, 4.48 mmol) and DIEA (1.564 mL, 8.95 mmol) in NMP (35 mL) was stirred for 2 min at rt, and then added to the resin. The resulting suspension was shaken for 16 h at rt and filtered and the resin was washed with DMA (3×). For capping, Ac₂O/pyridine/DMA (1:1:8) (40 mL) was added and the resulting suspension was shaken for 15 min at rt. The resin was drained, and then washed with DMA (3×).

Step 2-2. Fmoc-deprotection was done by repetitive treatment with 4-methylpiperidine/DMA (1:4) (3×30 mL, each time shaking for 15 min at rt). After Fmoc-removal, the resin was washed with DMA (3×) and DCM (5×). The product 150B (˜4.477 mmol) was taken to the next step.

### Step 3. PS-(2-Chlorotrityl) (2S,5S,8S,13S,16R)-2-amino-16-benzyl-8-(4-chlorobenzyl)-5-(methoxymethyl)-7,13,14-trimethyl-3,6,10,15-tetraoxo-4,7,11,14-tetraazaoctadecan-18-oate (150C)

Step 3-1. A solution of Fmoc-Ala-OH (E1) (4.18 g, 13.43 mmol), PyOxim (7.08 g, 13.43 mmol), and DIEA (4.69 mL, 26.9 mmol) in NMP (120 ml) was shaken at rt for 2 min, and was then added to 150B (4.477 mmol) that had been washed with NMP (3×). The resulting suspension was shaken for 4 h at rt and then filtered. A solution of Fmoc-Ala-OH (E1) (4.18 g, 13.43 mmol), PyOxim (7.08 g, 13.43 mmol) and DIEA (4.69 ml, 26.9 mmol) in NMP (120 ml) was stirred for 2 min at rt, was then added to the resin. The suspension was shaken for 16 h at rt, filtered, and the resin was washed with DMA (3×). For capping, Ac₂O/pyridine/DMA (1:1:8) (30 mL) was added and the resulting suspension was shaken for 15 min at rt. The resin was drained and then washed with DMA (3×).

Step 3-2. Fmoc-deprotection was done by repetitive treatment with 4-methylpiperidine/DMA (1:4) (3×30 mL, each time shaking for 15 min at rt). After Fmoc-removal the resin was washed with DMA (3×) and DCM (5×). The product 150C (assumed to be 4.477 mmol) was taken to the next step.

### Step 4. (2S,5S,8S,13S,16R)-2-amino-16-benzyl-8-(4-chlorobenzyl)-5-(methoxymethyl)-7,13,14-trimethyl-3,6,10,15-tetraoxo-4,7,11,14-tetraazaoctadecan-18-oic acid (150D)

To 150C (4.477 mmol) was added HFIP/DCM (1:3) (130 mL) and the resulting suspension was shaken for 20 min at rt. The cleavage solution was then filtered off and collected (3×). The resin was washed with DCM (2×) and the wash solutions were collected. The combined cleavage and wash solutions were concentrated to dryness in vacuo. The crude residue was lyophilized from tBuOH/H₂O (4:1) to yield 150D (3.9 g, ˜4.477 mmol; containing 76% of desired product) as a white solid. Analytical method 12: t_{R}=2.94 min; [M+H]⁺=660.3.

### Step 5. (3S,6S,9S,14S,17R)-17-benzyl-9-(4-chlorobenzyl)-1-(4-ethyl-2-(4-(1-methyl-2-(pyrrolidin-1-ylmethyl)-1H-imidazol-5-yl)phenoxy)phenyl)-6-(methoxymethyl)-3,8,14,15-tetramethyl-4,7,11,16-tetraoxo-2,5,8,12,15-pentaazanonadecan-19-oic acid trifluoroacetate (150E)

150D (130 mg, 150 μmol, 76% pure) and F5 (64.3 mg, 165 μmol) were dissolved in a mixture of DCM (10 mL) and AcOH (0.034 mL, 600 μmol) and the resulting solution was stirred for 1.5 h at rt. NaBH(OAc)₃ (159 mg, 750 μmol) was then added and the reaction mixture was stirred for 13.5 h at rt. MeOH (1.5 mL) was added and the reaction mixture was concentrated to dryness in vacuo. The crude product was purified by preparative reversed-phase HPLC (eluent A: 0.1% TFA in H₂O and eluent B: ACN). The pure fractions were combined and lyophilized to afford 150E (106.8 mg, 77.7 μmol, 52% yield) as a white solid. Analytical method 10; t_{R}=0.86 min; [M+H]⁺=1033.7.

### Step 6. (2S,5S,8S,13S,16R)-16-benzyl-8-(4-chlorobenzyl)-1-(4-ethyl-2-(4-(1-methyl-2-(pyrrolidin-1-ylmethyl)-1H-imidazol-5-yl)phenoxy)benzyl)-5-(methoxymethyl)-2,7,13,14-tetramethyl-1,4,7,11,14-pentaazacyclooctadecane-3,6,10,15,18-pentaone hydrochloride (Compound 150)

To a solution of 150E (106.8 mg, 77.7 μmol), HATU (129 mg, 0.339 mmol) and HOAt (17.28 mg, 0.127 mmol) in DCM (100 mL) was added 2,6-Lutidine (0.296 mL, 2.54 mmol) and the resulting mixture was stirred for 17 h at 40° C. Most of DCM was then removed in vacuo and the resulting residue was partitioned between EtOAc (70 mL) and 5% aq. NaHCO₃ (15 mL). The organic layer was washed with 5% aq. NaHCO₃ (3×10 mL) and brine (10 mL), dried over Na₂SO₄, filtered, and evaporated to dryness.

The crude product was purified by preparative reversed-phase HPLC (eluent A: 0.1% TFA in H₂O and eluent B: ACN). Pure fractions were combined and lyophilized to afford the TFA salt of the product (50 mg). The TFA salt was partitioned between EtOAc (30 mL) and 5% aq. NaHCO₃ (3 mL). The organic layer was washed with 5% aq. NaHCO₃ (2×2 mL) and brine (3 mL), dried over Na₂SO₄, filtered and evaporated to dryness. The residue was dissolved in ACN/H₂O (1:1) (20 mL) and 1 M HCl (0.13 mL), then lyophilized to afford Compound 150 (40.6 mg, 0.037 mmol, 47% yield) as a white solid. Analytical method 9; t_{R}=5.03 min; [M+H]⁺=1015.5.

The Compounds and intermediates shown in Table 1 below were prepared according to the procedure used for Compound 150 (Example 2.1) from the respective intermediates shown in Table 20, Table 22, Table 23, Table 24, and Table 25.

TABLE 1

| Cmd | | Synthesis | |
|---|---|---|---|
| No. | Structure | procedure | LCMS |
| 5 | 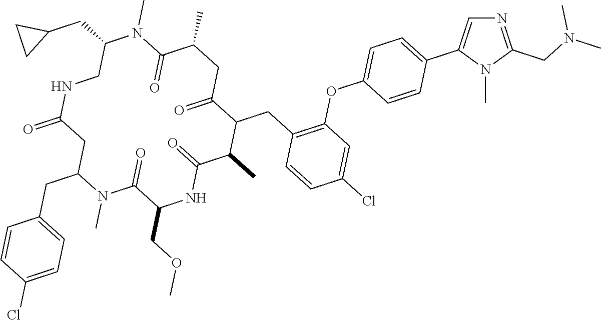 [SMILES: COC[C@@H]1NC(=O)[C@H](C)C(Cc2ccc(Cl)cc2Oc2ccc(-c3cnc(CN(C)C)n3C)cc2)C(=O)C[C@@H](C)C(=O)N(C)[C@@H](CC2CC2)CNC(=O)CC(Cc2ccc(Cl)cc2)N(C)C1=O] | Compound 150 (Example 2.1) | Analytical method 7 t_{R} = 1.05 min [M + H]⁺ = 959.7 |
| 7 | 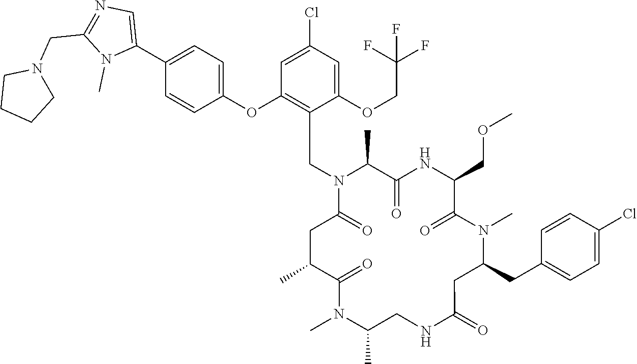 [SMILES: COC[C@@H]1NC(=O)[C@H](C)N(Cc2c(OCC(F)(F)F)cc(Cl)cc2Oc2ccc(-c3cnc(CN4CCCC4)n3C)cc2)C(=O)C[C@@H](C)C(=O)N(C)[C@@H](C)CNC(=O)C[C@H](Cc2ccc(Cl)cc2)N(C)C1=O] | Compound 150 (Example 2.1) | Analytical method 9 t_{R} = 4.51 min [M + H]⁺ = 1043.4 |
| 8 | 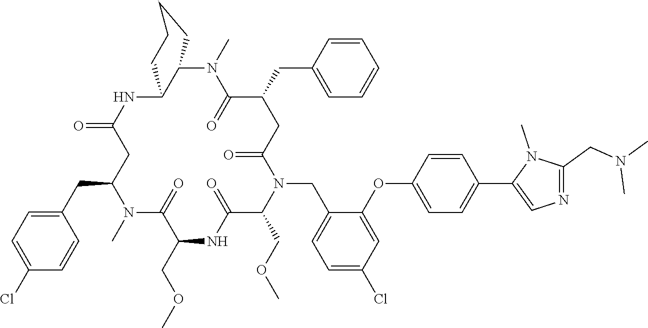 | Compound 150 (Example 2.1) | Analytical method 3 t_{R} = 1.12 min [M + H]⁺ = 1065.48 |
| 10 | 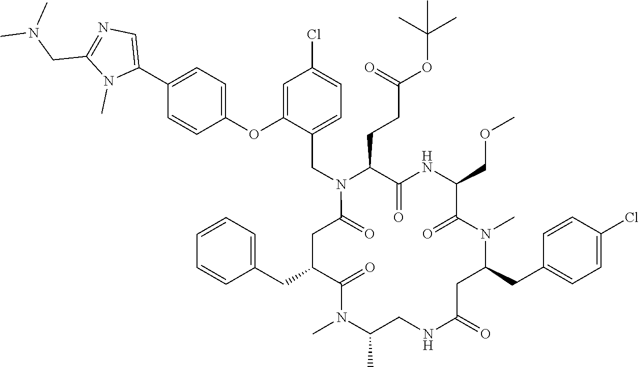 [SMILES: COC[C@@H]1NC(=O)[C@H](CCC(=O)OC(C)(C)C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 5 t_{R} = 1.28 min [M + H]⁺ = 1109.40 |
| 11A | 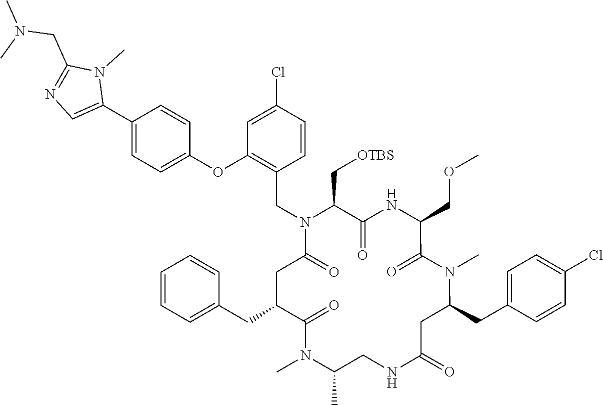 [SMILES: COC[C@@H]1NC(=O)[C@H](CO[Si](C)(C)C(C)(C)C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 5 t_{R} = 1.47 min [M + H]⁺ = 1125.9 |
| 12 | 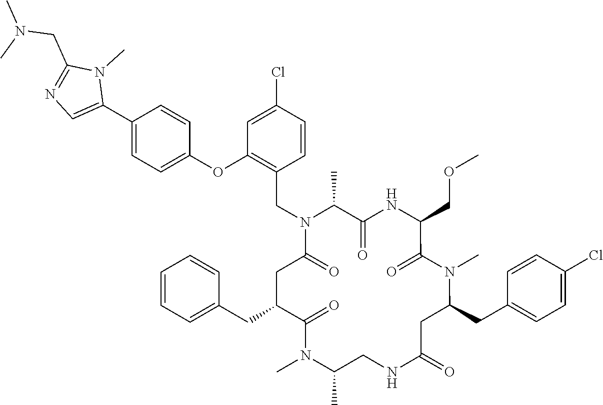 [SMILES: COC[C@@H]1NC(=O)[C@@H](C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 7 t_{R} = 0.98 min [M + H]⁺ = 995.5 |
| 15 | 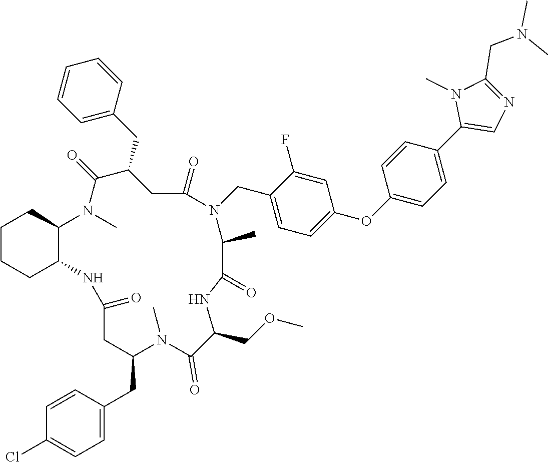 [SMILES: COC[C@@H]1NC(=O)[C@H](C)N(Cc2ccc(Oc3ccc(-c4cnc(CN(C)C)n4C)cc3)cc2F)C(=O)C[C@@H](Cc2ccccc2)C(=O)N(C)[C@@H]2CCCC[C@H]2NC(=O)C[C@H](Cc2ccc(Cl)cc2)N(C)C1=O] | Compound 150 (Example 2.1) | Analytical method 3 t_{R} = 1.07 min [M + H]⁺ = 1019.5 |
| 16 | 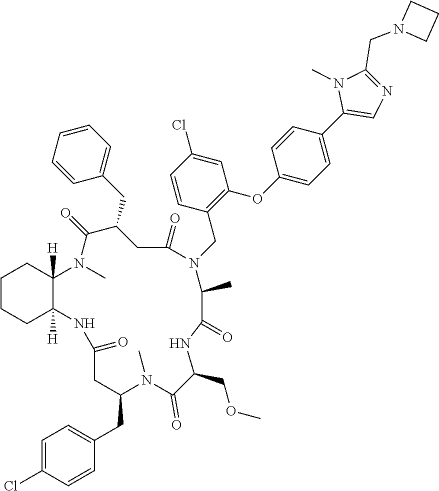 [SMILES: COC[C@@H]1NC(=O)[C@H](C)N(Cc2ccc(Cl)cc2Oc2ccc(-c3cnc(CN4CCC4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 7 t_{R} = 1.04 min [M + H]⁺ = 1049.3 |
| 17 | 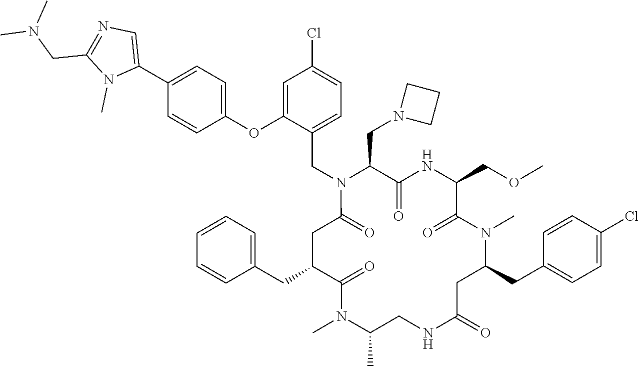 [SMILES: COC[C@@H]1NC(=O)[C@H](CN2CCC2)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 5 t_{R} = 1.25 min [M + H]⁺ = 1051.0 |
| 19 | 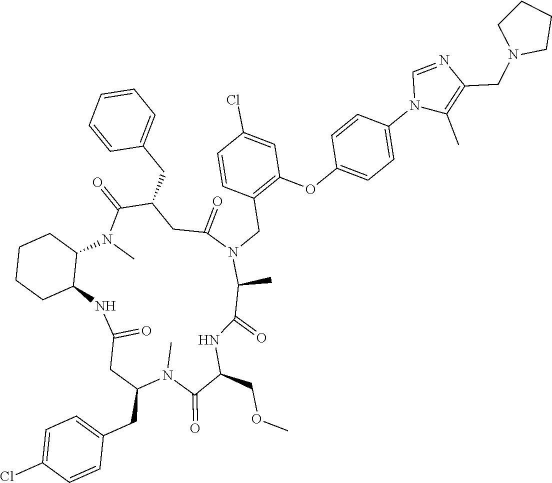 [SMILES: COC[C@@H]1NC(=O)[C@H](C)N(Cc2ccc(Cl)cc2Oc2ccc(-n3cnc(CN4CCCC4)c3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 2 t_{R} = 3.43 min [M + H]⁺ = 1061.3 |
| 22 | 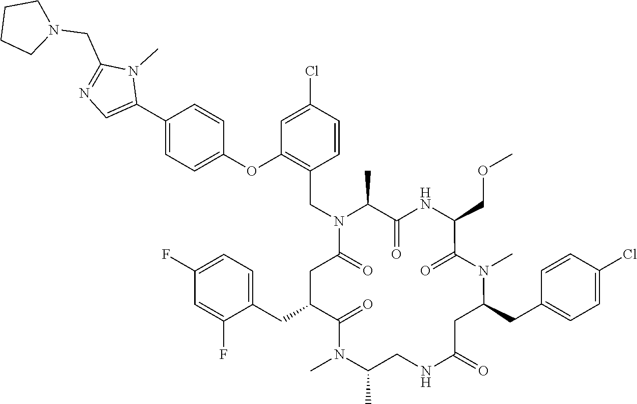 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccc(F)cc2F)C(=O)N(C)[C@@H](C)CNC(=O)C[C@H](Cc2ccc(Cl)cc2)N(C)C1=O] | Compound 150 (Example 2.1) | Analytical method 9 t_{R} = 4.77 min [M + H]⁺ = 1057.4 |
| 23 | 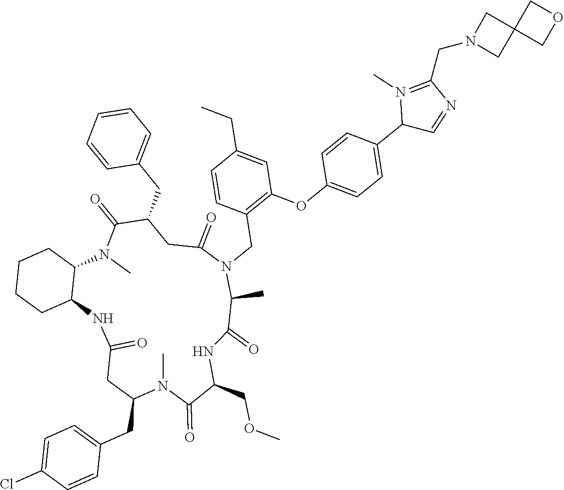 | Compound 150 (Example 2.1) | Analytical method 2 t_{R} = 2.89 min [M + H]⁺ = 1084.1 |
| 25 | 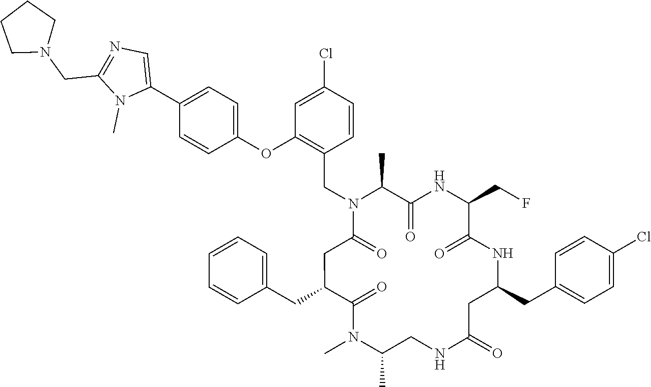 [SMILES: C[C@H]1CNC(=O)C[C@H](Cc2ccc(Cl)cc2)NC(=O)[C@H](CF)NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N1C] | Compound 150 (Example 2.1) | Analytical method 9 t_{R} = 4.95 min [M + H]⁺ = 995.4 |
| 26 | 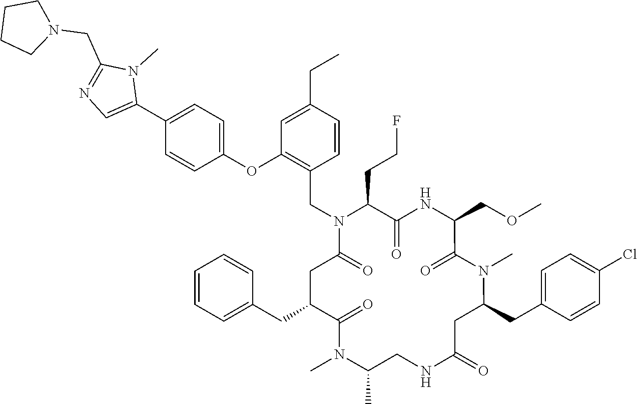 [SMILES: CCc1ccc(CN2C(=O)C[C@@H](Cc3ccccc3)C(=O)N(C)[C@@H](C)CNC(=O)C[C@H](Cc3ccc(Cl)cc3)N(C)C(=O)[C@H](COC)NC(=O)[C@@H]2CCF)c(Oc2ccc(-c3cnc(CN4CCCC4)n3C)cc2)c1] | Compound 150 (Example 2.1) | Analytical method 5 t_{R} = 1.24 min [M + H]⁺ = 1047.5 |
| 27 | 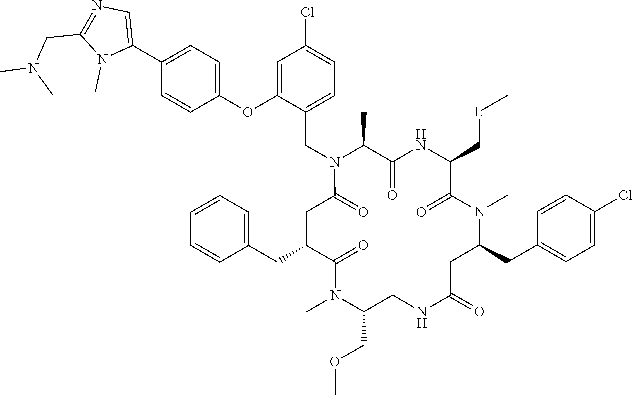 | Compound 150 (Example 2.1) | Analytical method 6 t_{R} = 2.28 min [M + H]⁺ = 1025.5 |
| 28 | 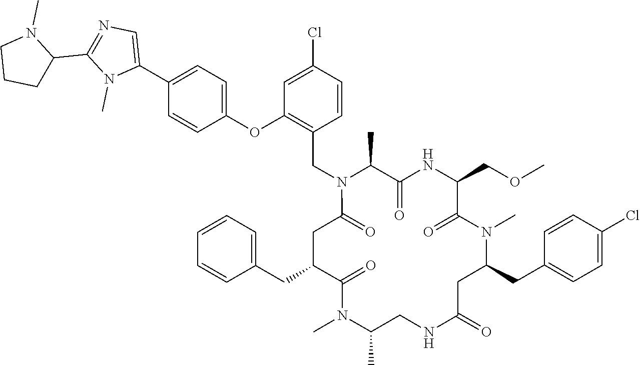 [SMILES: COC[C@@H]1NC(=O)[C@H](C)N(Cc2ccc(Cl)cc2Oc2ccc(-c3cnc(C4CCCN4C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.42 min [M + H]⁺ = 1021.4 |
| 29 | 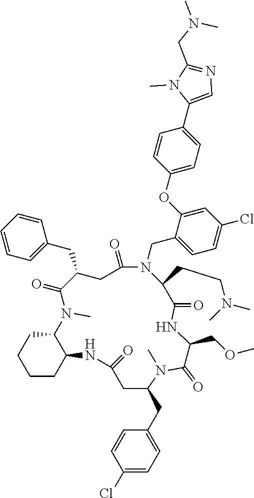 [SMILES: COC[C@@H]1NC(=O)[C@H](CCN(C)C)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1)/ Polymer-bound pentamers were synthesized with a N-terminal Boc group. After cleavage from resin with HFIP, Boc was removed by treatment with TFA. | Analytical method 3 t_{R} = 0.89 min [M + H]⁺ = 1091.5 |
| 30 | 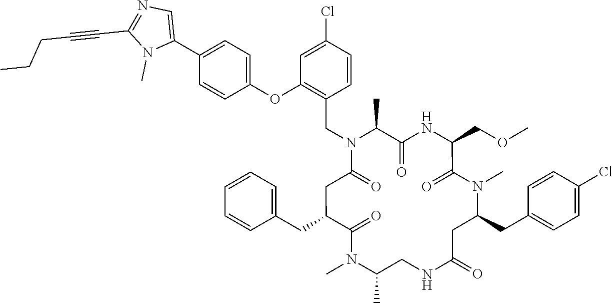 [SMILES: CCCC#Cc1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)n1C] | Compound 150 (Example 2.1) | Analytical method 9 t_{R} = 5.65 min [M + H]⁺ = 1004.4 |
| 32 | 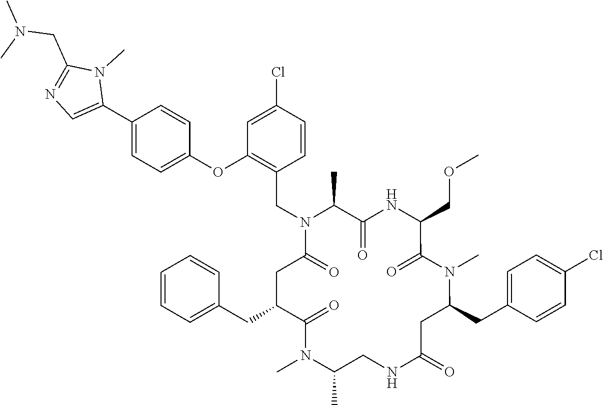 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 7 t_{R} = 1.00 min [M + H]⁺ = 995.4 |
| 34 | 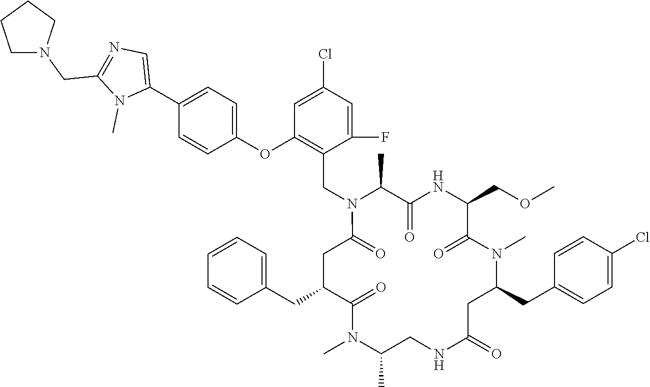 [SMILES: COC[C@@H]1NC(=O)[C@H](C)N(Cc2c(F)cc(Cl)cc2Oc2ccc(-c3cnc(CN4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.40 min [M + H]⁺ = 1039.4 |
| 35 | 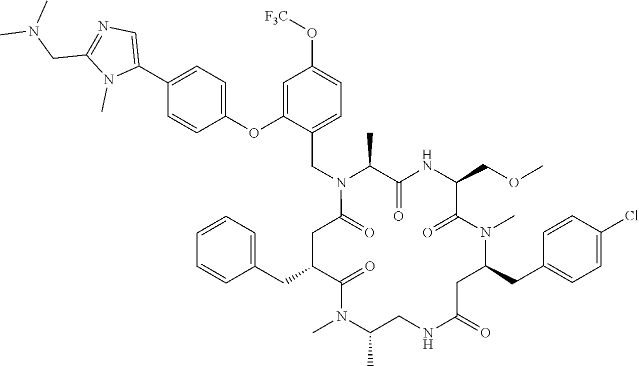 [SMILES: COC[C@@H]1NC(=O)[C@H](C)N(Cc2ccc(OC(F)(F)F)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 3 t_{R} = 1.07 min [M + H]⁺ = 1045.5 |
| 36A | 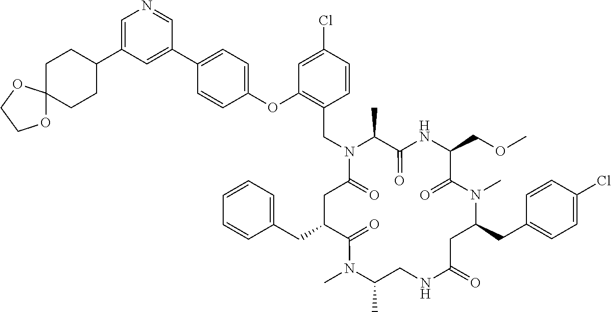 [SMILES: COC[C@@H]1NC(=O)[C@H](C)N(Cc2ccc(Cl)cc2Oc2ccc(-c3cncc(C4CCC5(CC4)OCCO5)c3)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.39 min [M + H]⁺ = 1075.7 |
| 40 | 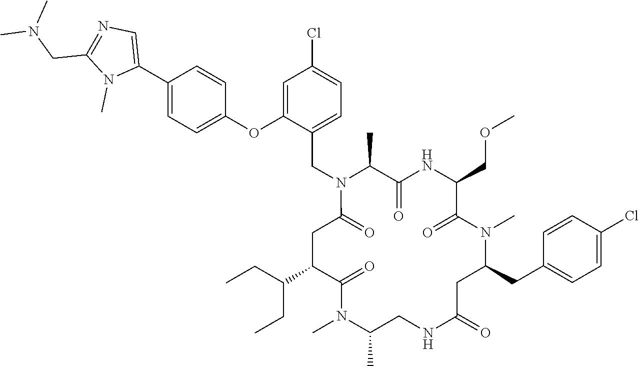 [SMILES: CCC(CC)[C@@H]1CC(=O)N(Cc2ccc(Cl)cc2Oc2ccc(-c3cnc(CN(C)C)n3C)cc2)[C@@H](C)C(=O)N[C@@H](COC)C(=O)N(C)[C@@H](Cc2ccc(Cl)cc2)CC(=O)NC[C@H](C)N(C)C1=O] | Compound 150 (Example 2.1) | Analytical method 4 t_{R} = 2.12 min [M + H]⁺ = 974.9 |
| 41 | 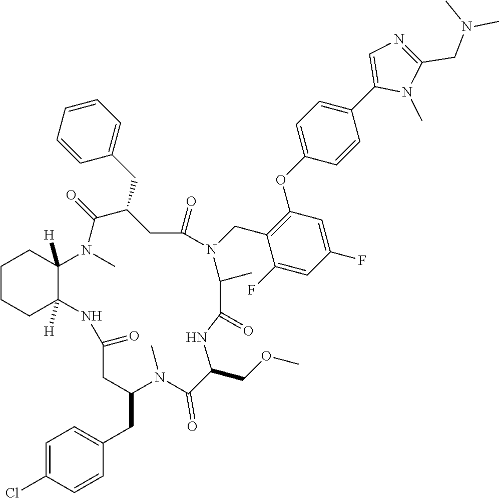 [SMILES: COC[C@@H]1NC(=O)C(C)N(Cc2c(F)cc(F)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 7 t_{R} = 0.97 min [M + H]⁺ = 1037.4 |
| 42 | 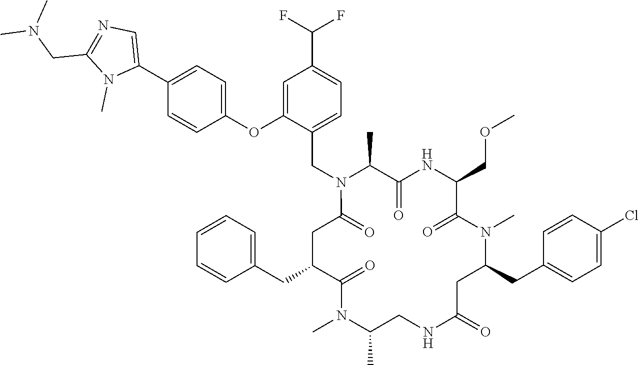 [SMILES: COC[C@@H]1NC(=O)[C@H](C)N(Cc2ccc(C(F)F)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 3 t_{R} = 1.02 min [M + H]⁺ = 1011.5 |
| 43A | 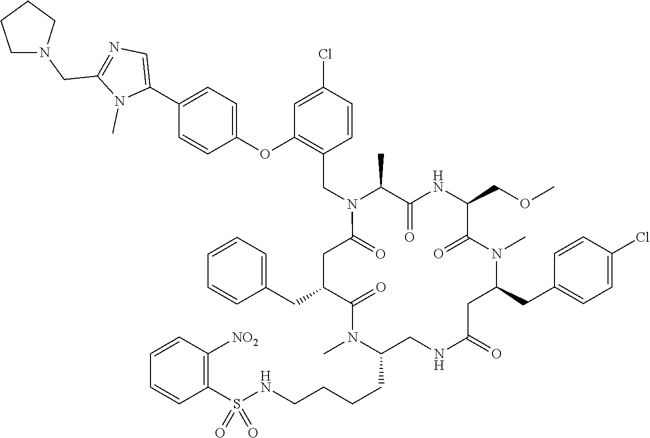 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](CCCCNS(=O)(=O)c2ccccc2[N+](=O)[O-])CNC(=O)C[C@H](Cc2ccc(Cl)cc2)N(C)C1=O] | Compound 150 (Example 2.1) | Analytical method 9 t_{R} = 5.26 min [M + H]⁺ = 1263.5 |
| 46 | 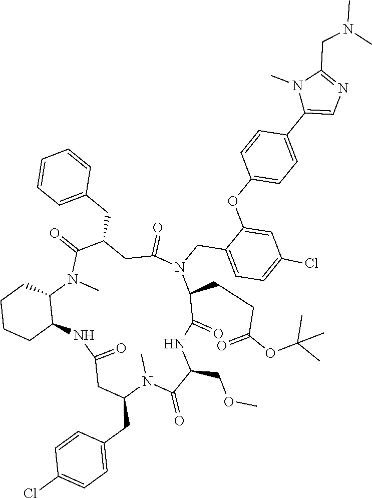 [SMILES: COC[C@@H]1NC(=O)[C@H](CCC(=O)OC(C)(C)C)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 6 t_{R} = 2.61 min [M + H]⁺ = 1149.5 |
| 48 | 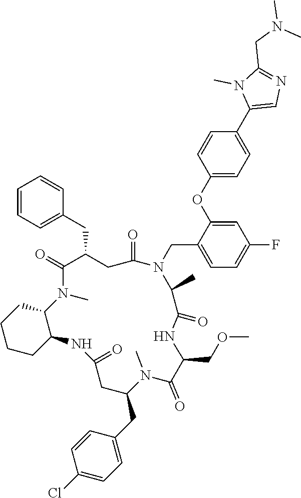 [SMILES: COC[C@@H]1NC(=O)[C@H](C)N(Cc2ccc(F)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 3 t_{R} = 1.07 min [M + H]⁺ = 1019.5 |
| 51 | 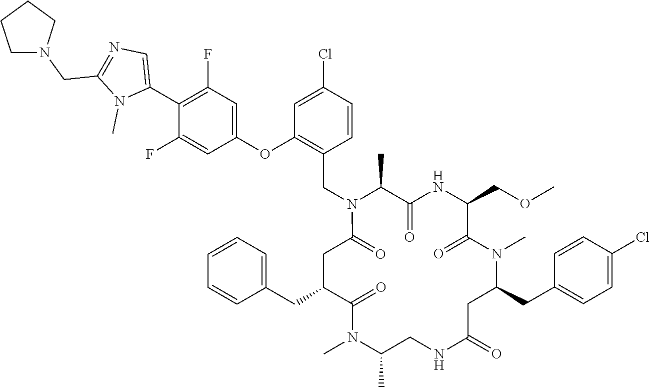 [SMILES: COC[C@@H]1NC(=O)[C@H](C)N(Cc2ccc(Cl)cc2Oc2cc(F)c(-c3cnc(CN4CCCC4)n3C)c(F)c2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.05 min [M + H]⁺ = 1057.5 |
| 54 | 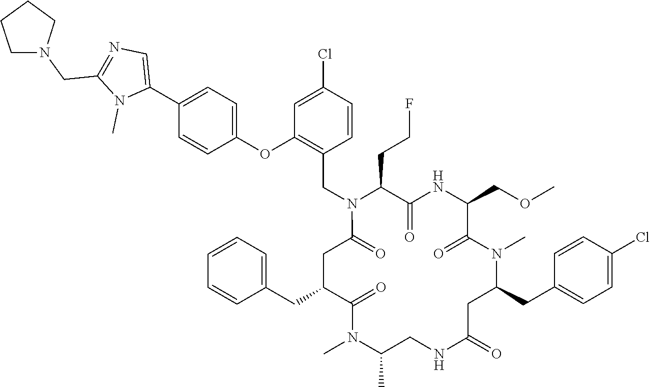 [SMILES: COC[C@@H]1NC(=O)[C@H](CCF)N(Cc2ccc(Cl)cc2Oc2ccc(-c3cnc(CN4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 9 t_{R} = 4.76 min [M + H]⁺ = 1053.4 |
| 55 | 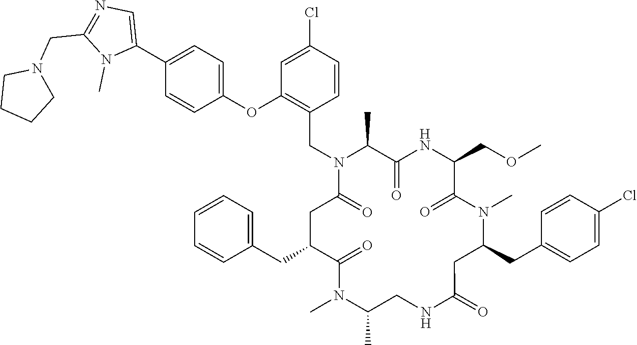 | Compound 150 (Example 2.1) | Analytical method 13 t_{R} = 4.14 min [M + H]⁺ = 1035.5 |
| 58 | 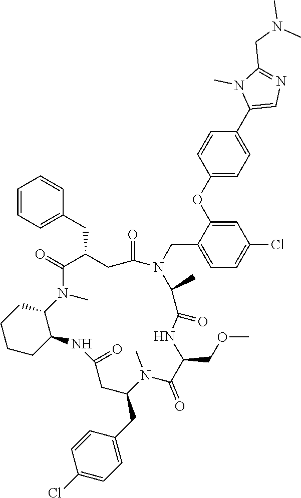 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 6 t_{R} = 2.46 min [M + H]⁺ = 1035.5 |
| 60 | 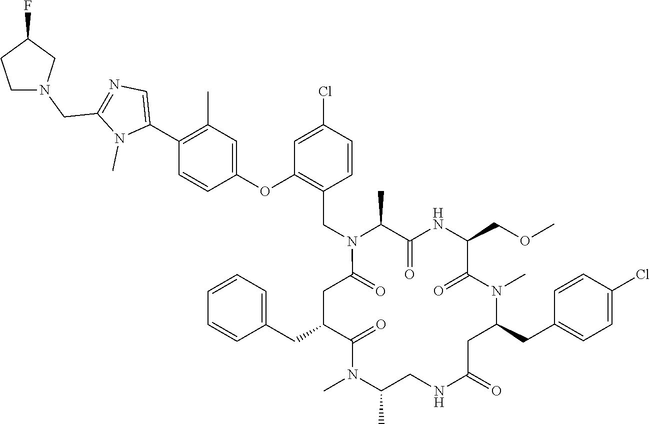 | Compound 150 (Example 2.1) | Analytical method 13 t_{R} = 4.48 min [M + H]⁺ = 1039.4 |
| 62 | 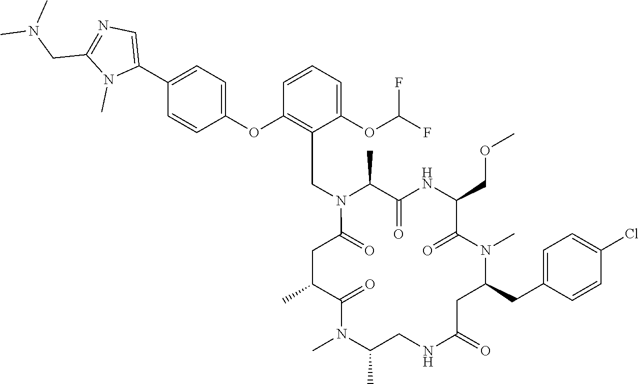 [SMILES: COC[C@@H]1NC(=O)[C@H](C)N(Cc2c(Oc3ccc(-c4cnc(CN(C)C)n4C)cc3)cccc2OC(F)F)C(=O)C[C@@H](C)C(=O)N(C)[C@@H](C)CNC(=O)C[C@H](Cc2ccc(Cl)cc2)N(C)C1=O] | Compound 150 (Example 2.1) | Analytical method 7 t_{R} = 0.95 min [M + H]⁺ = 951.3 |
| 63 | 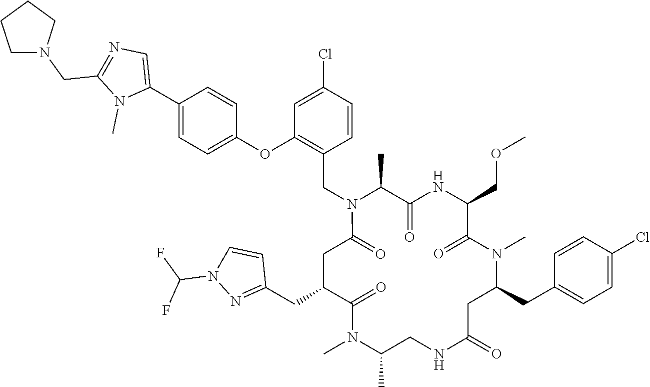 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccn(C(F)F)n2)C(=O)N(C)[C@@H](C)CNC(=O)C[C@H](Cc2ccc(Cl)cc2)N(C)C1=O] | Compound 150 (Example 2.1) | Analytical method 14 t_{R} = 4.08 min [M + H]⁺ = 1061.4 |
| 64 | 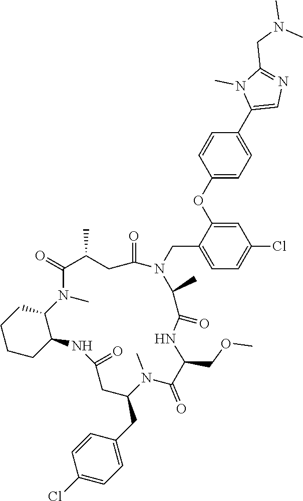 [SMILES: COC[C@@H]1NC(=O)[C@H](C)N(Cc2ccc(Cl)cc2Oc2ccc(-c3cnc(CN(C)C)n3C)cc2)C(=O)C[C@@H](C)C(=O)N(C)[C@H]2CCCC[C@@H]2NC(=O)C[C@H](Cc2ccc(Cl)cc2)N(C)C1=O] | Compound 150 (Example 2.1) | Analytical method 6 t_{R} = 2.02 min [M + H]⁺ = 959.4 |
| 67 | 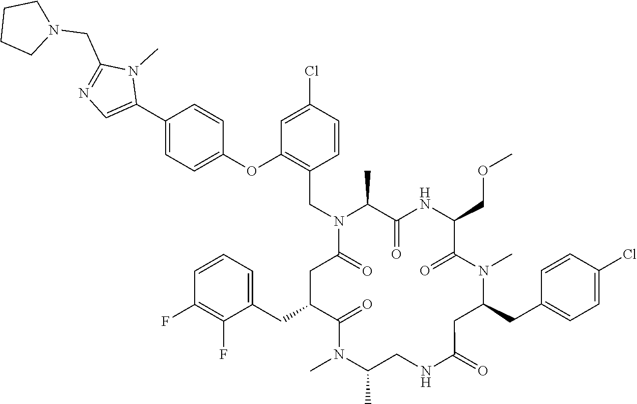 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2cccc(F)c2F)C(=O)N(C)[C@@H](C)CNC(=O)C[C@H](Cc2ccc(Cl)cc2)N(C)C1=O] | Compound 150 (Example 2.1) | Analytical method 9 t_{R} = 4.77 min [M + H]⁺ = 1057.4 |
| 68 | 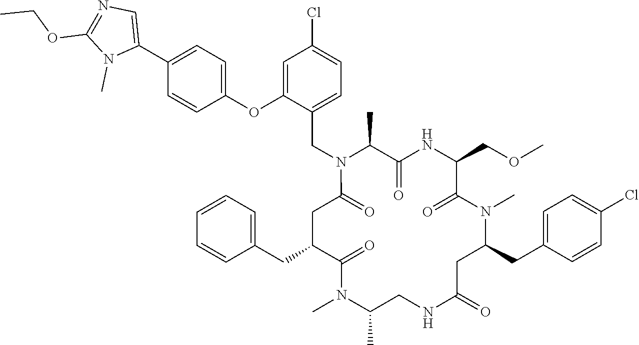 [SMILES: CCOc1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)n1C] | Compound 150 (Example 2.1) | Analytical method 4 t_{R} = 2.43 min [M + H]⁺ = 982.4 |
| 70 | 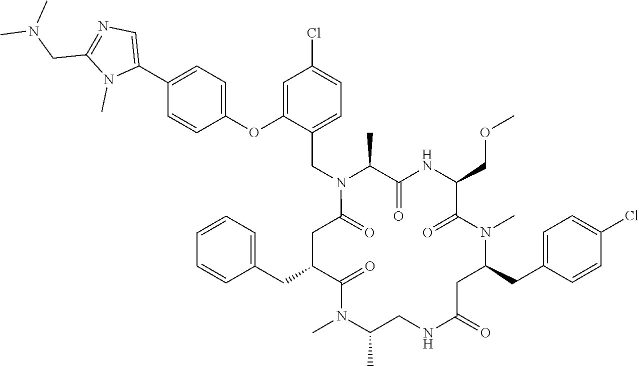 | Compound 150 (Example 2.1) | Analytical method 6 t_{R} = 2.28 min [M + H]⁺ = 1009.5 |
| 71 | 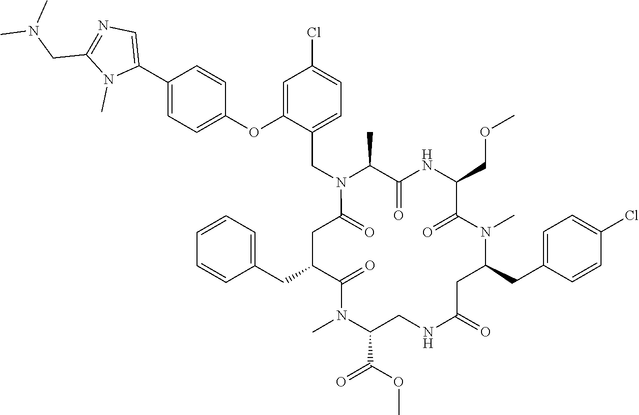 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H](C(=O)OC)CNC(=O)C[C@H](Cc2ccc(Cl)cc2)N(C)C1=O] | Compound 150 (Example 2.1)/ starting from ABC trimer K1 which was synthesized by loading of Fmoc- protected K1 trimer (see Example 15.1) on PS-2- Chlorotrityl chloride followed by Fmoc-removal. | Analytical method 4 t_{R} = 2.09 min [M + 2H]²⁺ = 520.3 |
| 72 | 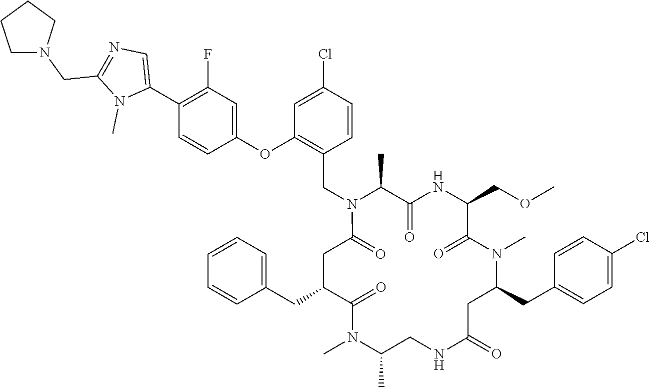 [SMILES: COC[C@@H]1NC(=O)[C@H](C)N(Cc2ccc(Cl)cc2Oc2ccc(-c3cnc(CN4CCCC4)n3C)c(F)c2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.46 min [M + H]⁺ = 1039.5 |
| 73 | 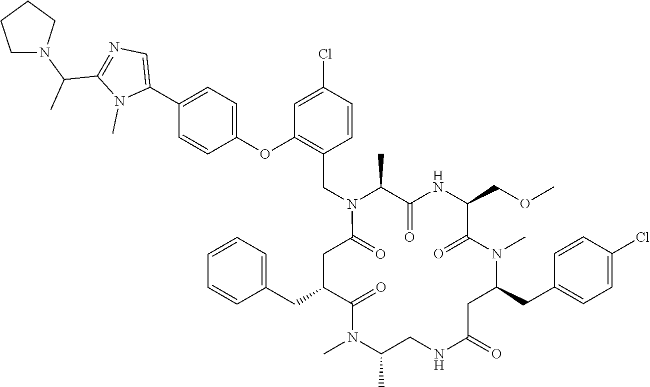 [SMILES: COC[C@@H]1NC(=O)[C@H](C)N(Cc2ccc(Cl)cc2Oc2ccc(-c3cnc(C(C)N4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 9 t_{R} = 5.06 min [M + H]⁺ = 1035.5 |
| 74 | 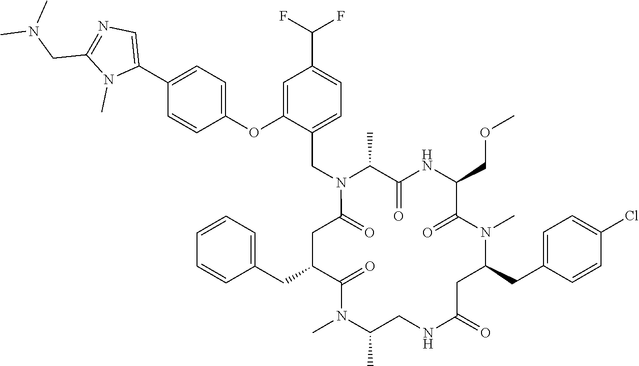 [SMILES: COC[C@@H]1NC(=O)[C@@H](C)N(Cc2ccc(C(F)F)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 3 t_{R} = 1.00 min [M + H]⁺ = 1011.5 |
| 75A | 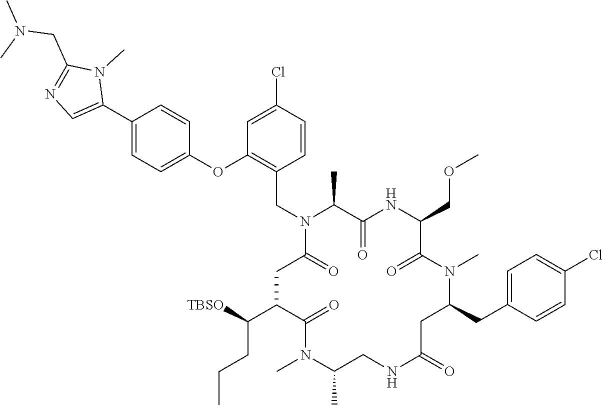 | PS-AB1 was coupled with Fmoc- protected CDE trimer J1 (see Example 14.1) with remaining steps performed according to the procedure used for Compound 150 (Example 2.1) | Analytical method 2 t_{R} = 3.62 min [M + H]⁺ = 1091.4 |
| 76 | 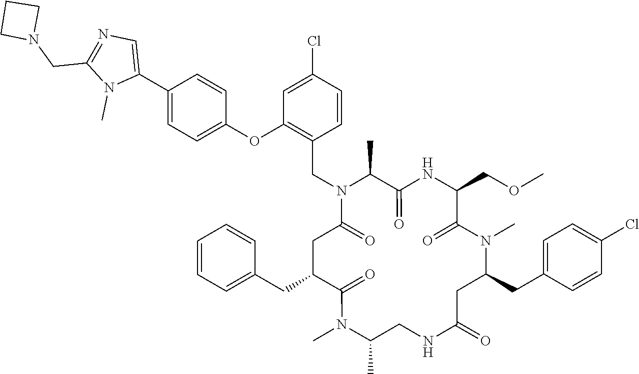 [SMILES: COC[C@@H]1NC(=O)[C@H](C)N(Cc2ccc(Cl)cc2Oc2ccc(-c3cnc(CN4CCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 2 t_{R} = 2.80 min [M + H]⁺ = 1007.5 |
| 78 | 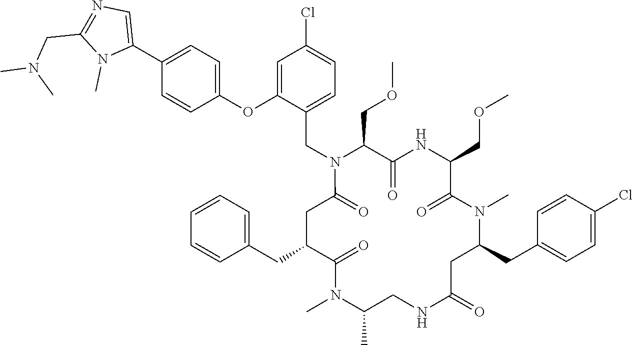 [SMILES: COC[C@@H]1NC(=O)[C@H](COC)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 6 t_{R} = 2.24 min [M + H]⁺ = 1025.5 |
| 80 | 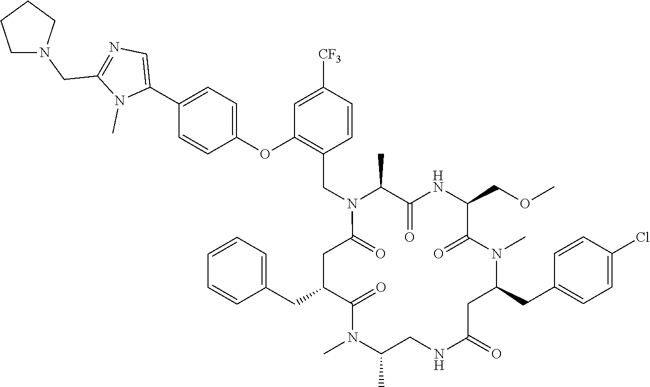 [SMILES: COC[C@@H]1NC(=O)[C@H](C)N(Cc2ccc(C(F)(F)F)cc2Oc2ccc(-c3cnc(CN4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 9 t_{R} = 4.79 min [M + H]⁺ = 1055.5 |
| 81 | 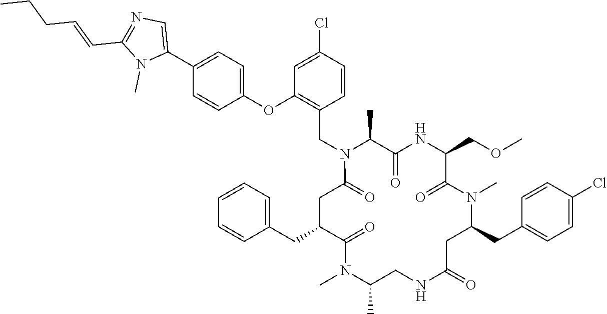 [SMILES: CCC/C=C/c1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)n1C] | Compound 150 (Example 2.1) | Analytical method 9 t_{R} = 5.26 min [M + H]⁺ = 1006.4 |
| 82A | 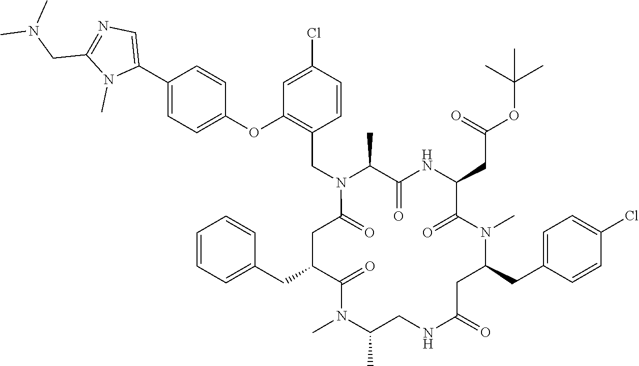 [SMILES: C[C@H]1CNC(=O)C[C@H](Cc2ccc(Cl)cc2)N(C)C(=O)[C@H](CC(=O)OC(C)(C)C)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Compound 150 (Example 2.1) | Analytical method 6 t_{R} = 2.37 min [M + H]⁺ = 1065.5 |
| 83 | 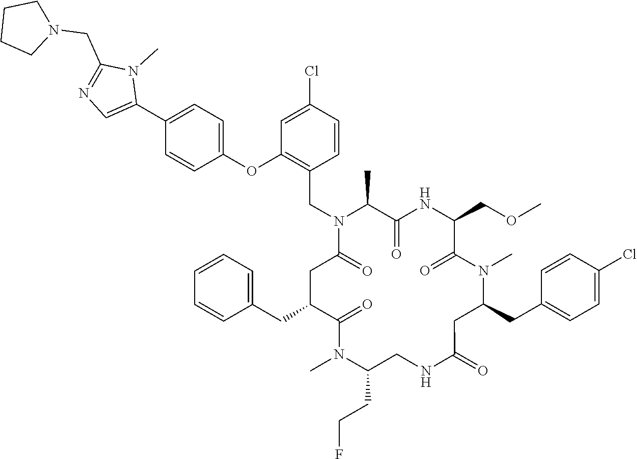 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](CCF)CNC(=O)C[C@H](Cc2ccc(Cl)cc2)N(C)C1=O] | Compound 150 (Example 2.1) | Analytical method 9 t_{R} = 4.81 min [M + H]⁺ = 1053.4 |
| 85 | 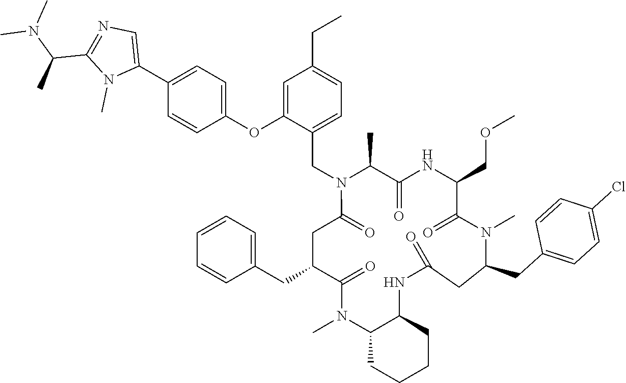 [SMILES: CCc1ccc(CN2C(=O)C[C@@H](Cc3ccccc3)C(=O)N(C)[C@H]3CCCC[C@@H]3NC(=O)C[C@H](Cc3ccc(Cl)cc3)N(C)C(=O)[C@H](COC)NC(=O)[C@@H]2C)c(Oc2ccc(-c3cnc([C@@H](C)N(C)C)n3C)cc2)c1] | Compound 150 (Example 2.1) | Analytical method 4 t_{R} = 2.27 min [M + H]⁺ = 1043.7 |
| 87 | 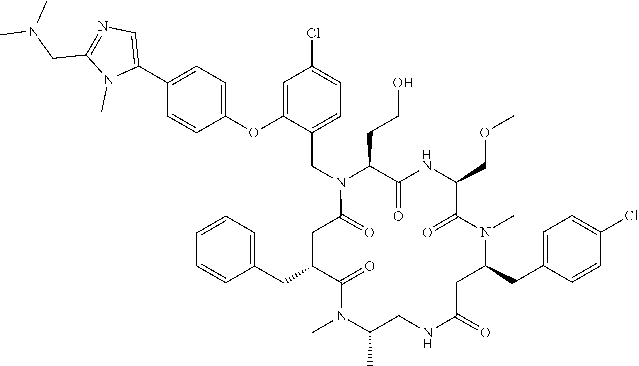 [SMILES: COC[C@@H]1NC(=O)[C@H](CCO)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 2 t_{R} = 2.90 min [M + 2H]²⁺ = 513.3 |
| 90 | 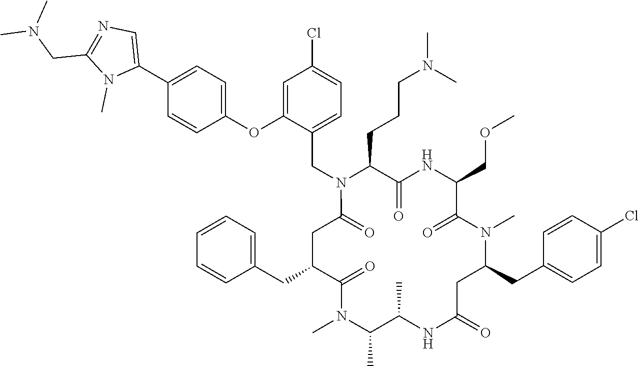 [SMILES: COC[C@@H]1NC(=O)[C@H](CCCN(C)C)N(Cc2ccc(Cl)cc2Oc2ccc(-c3cnc(CN(C)C)n3C)cc2)C(=O)C[C@@H](Cc2ccccc2)C(=O)N(C)[C@@H](C)[C@H](C)NC(=O)C[C@H](Cc2ccc(Cl)cc2)N(C)C1=O] | Compound 150 (Example 2.1) | Analytical method 3 t_{R} = 0.87 min [M + H]⁺ = 1080.5 |
| 92 | 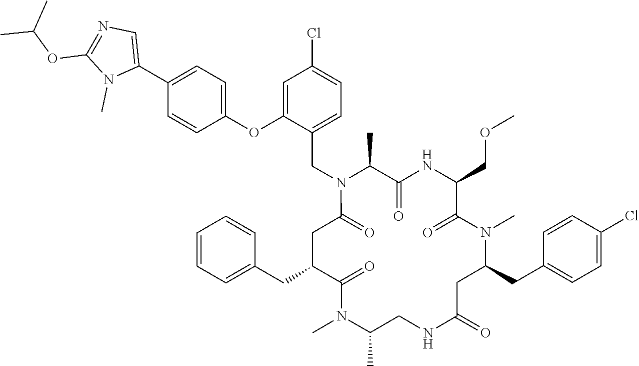 [SMILES: COC[C@@H]1NC(=O)[C@H](C)N(Cc2ccc(Cl)cc2Oc2ccc(-c3cnc(OC(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 3 t_{R} = 1.31 min [M + H]⁺ = 996.4 |
| 96 | 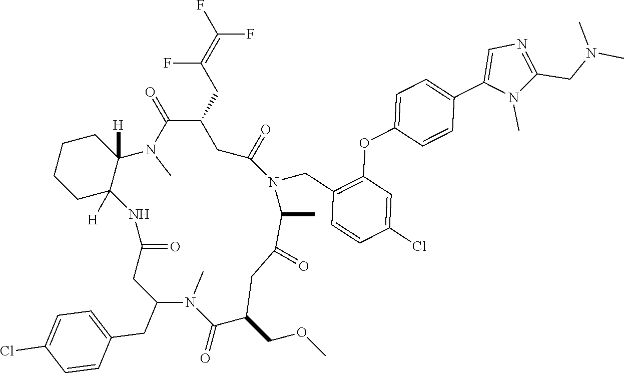 [SMILES: COC[C@@H]1CC(=O)[C@H](C)N(Cc2ccc(Cl)cc2Oc2ccc(-c3cnc(CN(C)C)n3C)cc2)C(=O)C[C@@H](CC(F)=C(F)F)C(=O)N(C)[C@H]2CCCCC2NC(=O)CC(Cc2ccc(Cl)cc2)N(C)C1=O] | Compound 150 (Example 2.1) | Analytical method 7 t_{R} = 1.04 min [M + H]⁺ = 1038.5 |
| 97 | 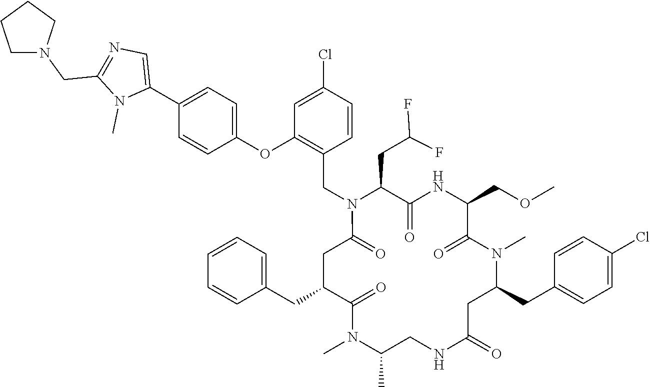 [SMILES: COC[C@@H]1NC(=O)[C@H](CC(F)F)N(Cc2ccc(Cl)cc2Oc2ccc(-c3cnc(CN4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 9 t_{R} = 4.98 min [M + H]⁺ = 1071.4 |
| 102 | 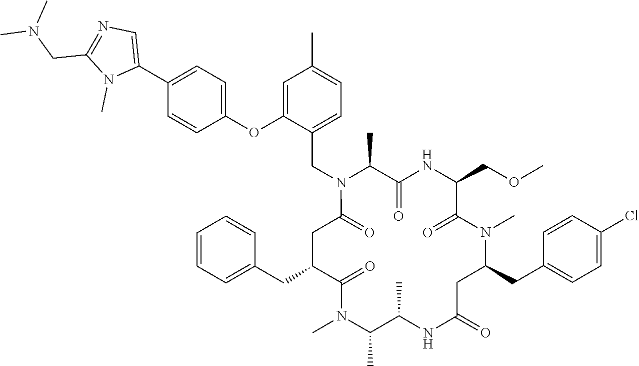 [SMILES: COC[C@@H]1NC(=O)[C@H](C)N(Cc2ccc(C)cc2Oc2ccc(-c3cnc(CN(C)C)n3C)cc2)C(=O)C[C@@H](Cc2ccccc2)C(=O)N(C)[C@@H](C)[C@H](C)NC(=O)C[C@H](Cc2ccc(Cl)cc2)N(C)C1=O] | Compound 150 (Example 2.1) | Analytical method 7 t_{R} = 1.03 min [M + H]⁺ = 989.5 |
| 103 | 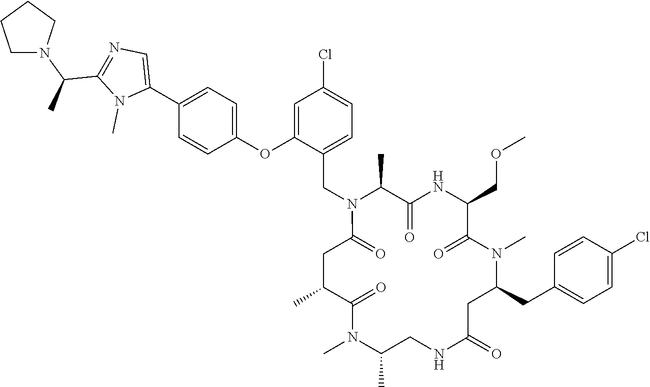 [SMILES: COC[C@@H]1NC(=O)[C@H](C)N(Cc2ccc(Cl)cc2Oc2ccc(-c3cnc([C@@H](C)N4CCCC4)n3C)cc2)C(=O)C[C@@H](C)C(=O)N(C)[C@@H](C)CNC(=O)C[C@H](Cc2ccc(Cl)cc2)N(C)C1=O] | Compound 150 (Example 2.1) | Analytical method 2 t_{R} = 2.85 min [M + H]⁺ = 959.6 |
| 104 | 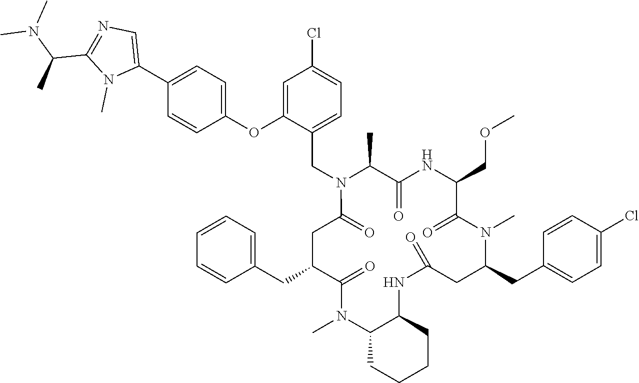 [SMILES: COC[C@@H]1NC(=O)[C@H](C)N(Cc2ccc(Cl)cc2Oc2ccc(-c3cnc([C@@H](C)N(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 2 t_{R} = 3.11 min [M + H]⁺ = 1049.8 |
| 107 | 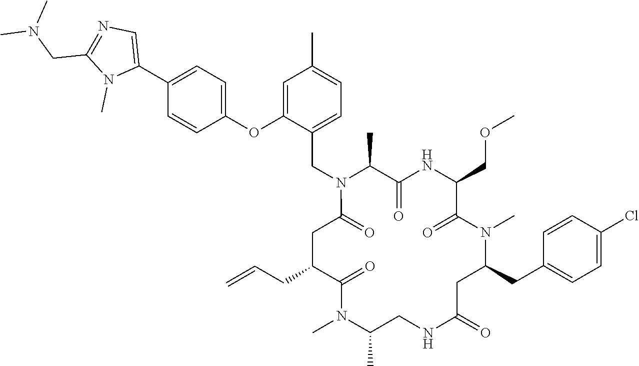 | Compound 150 (Example 2.1) | Analytical method 7 t_{R} = 1.00 min [M + H]⁺ = 939.0 |
| 108 | 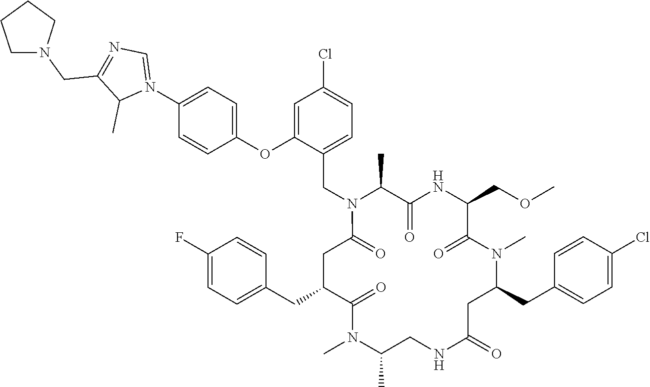 | Compound 150 (Example 2.1) | Analytical method 9 t_{R} = 4.74 min [M + H]⁺ = 1039.4 |
| 110 | 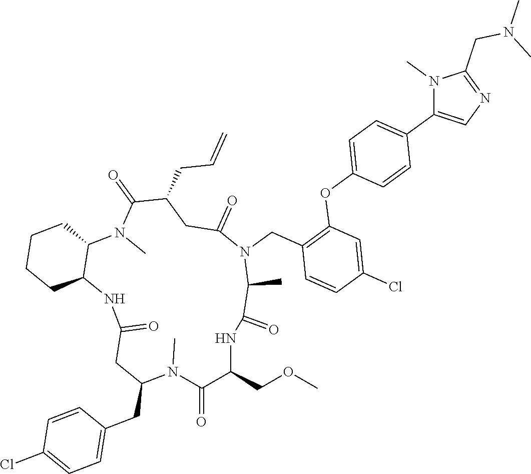 [SMILES: C=CC[C@@H]1CC(=O)N(Cc2ccc(Cl)cc2Oc2ccc(-c3cnc(CN(C)C)n3C)cc2)[C@@H](C)C(=O)N[C@@H](COC)C(=O)N(C)[C@@H](Cc2ccc(Cl)cc2)CC(=O)N[C@H]2CCCC[C@@H]2N(C)C1=O] | Compound 150 (Example 2.1) | Analytical method 7 t_{R} = 1.06 min [M + H]⁺ = 984.7 |
| 112 | 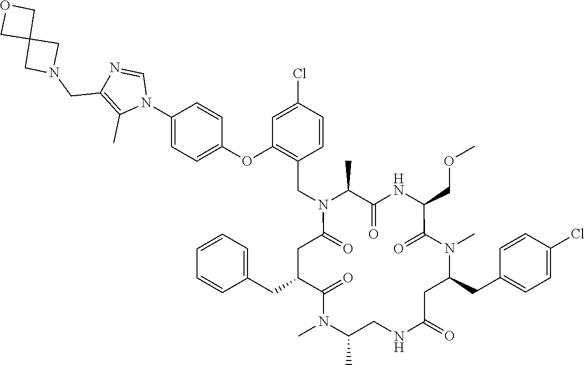 [SMILES: COC[C@@H]1NC(=O)[C@H](C)N(Cc2ccc(Cl)cc2Oc2ccc(-n3cnc(CN4CC5(COC5)C4)c3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 3 t_{R} = 1.03 min [M + H]⁺ = 1049.5 |
| 114A | 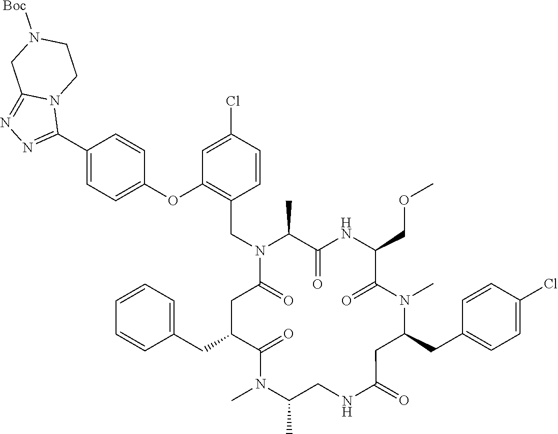 [SMILES: COC[C@@H]1NC(=O)[C@H](C)N(Cc2ccc(Cl)cc2Oc2ccc(-c3nnc4n3CCN(C(=O)OC(C)(C)C)C4)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.25 min [M + H]⁺ = 1080.7 |
| 115A | 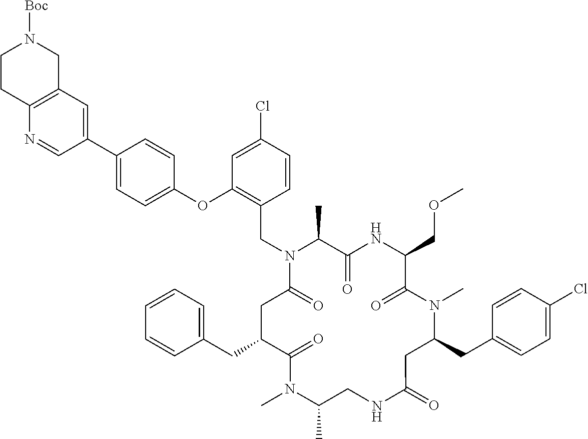 [SMILES: COC[C@@H]1NC(=O)[C@H](C)N(Cc2ccc(Cl)cc2Oc2ccc(-c3cnc4c(c3)CN(C(=O)OC(C)(C)C)CC4)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.43 min [M + H]⁺ = 1090.7 |
| 119A | 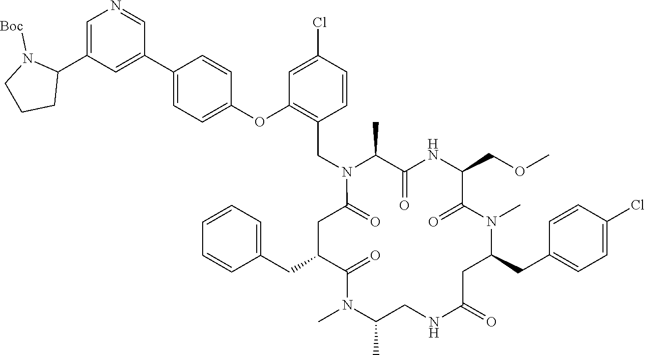 [SMILES: COC[C@@H]1NC(=O)[C@H](C)N(Cc2ccc(Cl)cc2Oc2ccc(-c3cncc(C4CCCN4C(=O)OC(C)(C)C)c3)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.41 min [M + H]⁺ = 1104.6 |
| 121A | 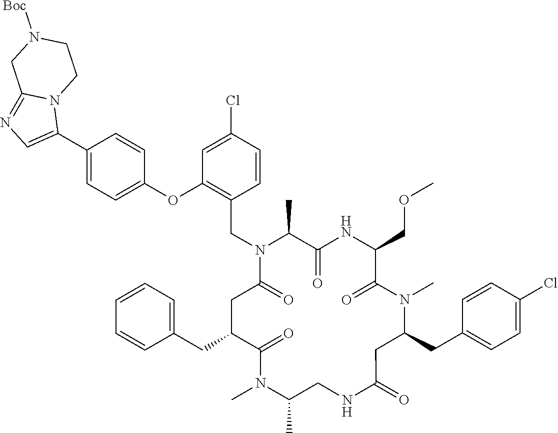 [SMILES: COC[C@@H]1NC(=O)[C@H](C)N(Cc2ccc(Cl)cc2Oc2ccc(-c3cnc4n3CCN(C(=O)OC(C)(C)C)C4)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.26 min [M + H]⁺ = 1079.6 |
| 122A | 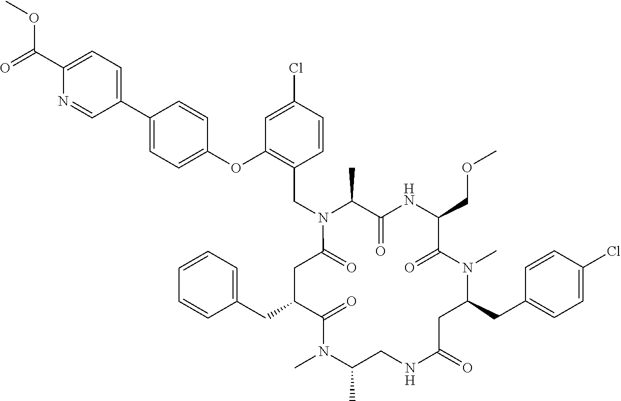 [SMILES: COC[C@@H]1NC(=O)[C@H](C)N(Cc2ccc(Cl)cc2Oc2ccc(-c3ccc(C(=O)OC)nc3)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.31 min [M + H]⁺ = 993.8 |
| 126 | 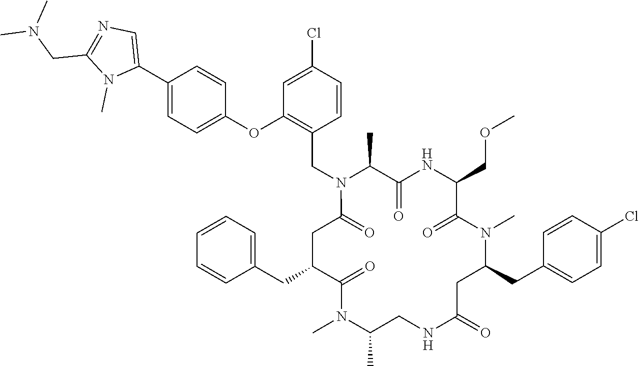 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.30 min [M + H]⁺ = 995.4 |
| 135 | 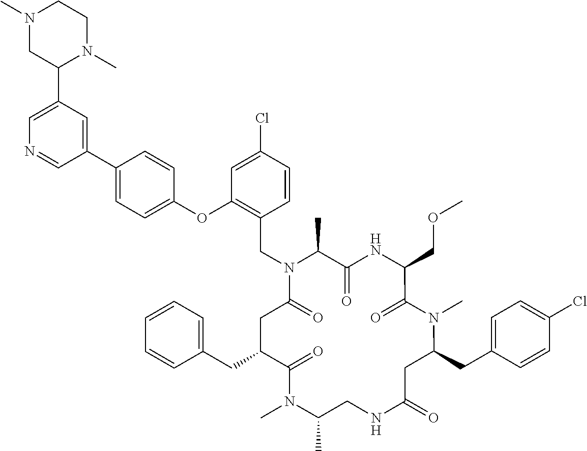 [SMILES: COC[C@@H]1NC(=O)[C@H](C)N(Cc2ccc(Cl)cc2Oc2ccc(-c3cncc(C4CN(C)CCN4C)c3)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.28 min [M + H]⁺ = 1047.6 |
| 136A | 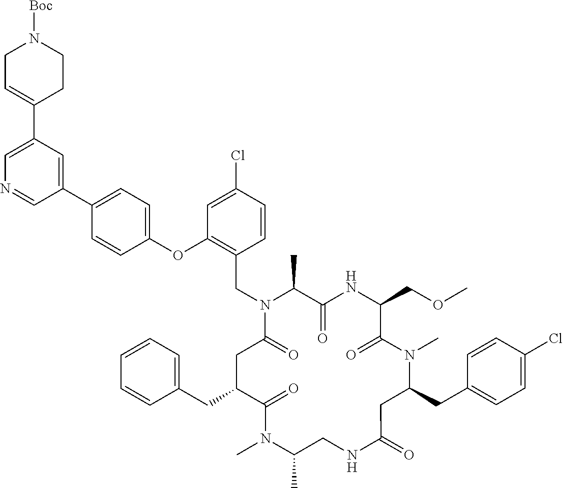 [SMILES: COC[C@@H]1NC(=O)[C@H](C)N(Cc2ccc(Cl)cc2Oc2ccc(-c3cncc(C4=CCN(C(=O)OC(C)(C)C)CC4)c3)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.47 min [M + H]⁺ = 1116.7 |
| 137A | 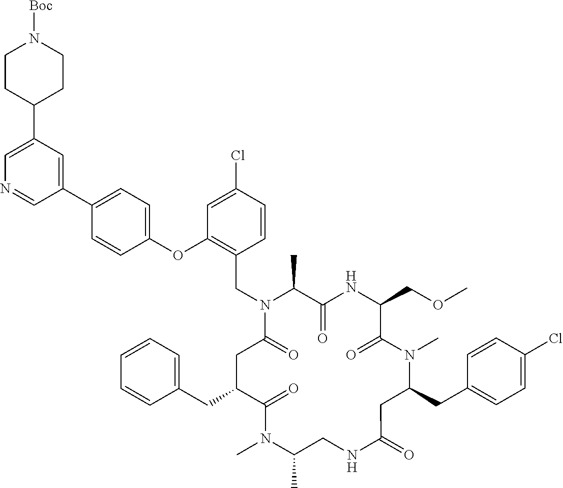 [SMILES: COC[C@@H]1NC(=O)[C@H](C)N(Cc2ccc(Cl)cc2Oc2ccc(-c3cncc(C4CCN(C(=O)OC(C)(C)C)CC4)c3)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 10 t_{R} = 1.47 min [M + H]⁺ = 1118.7 |
| 138 | 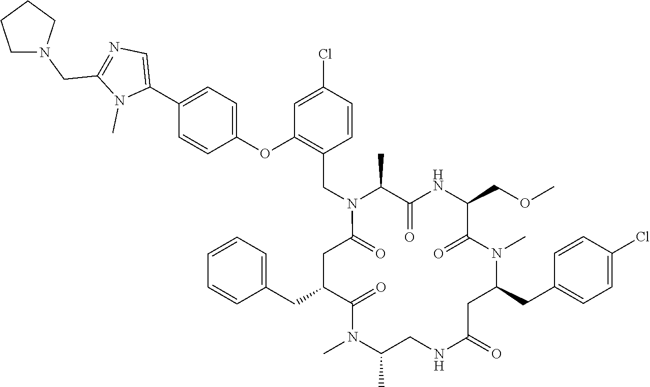 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.34 min [M + H]⁺ = 1021.5 |
| 141 | 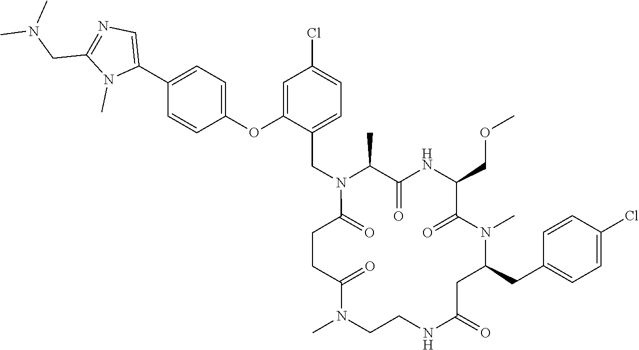 [SMILES: COC[C@@H]1NC(=O)[C@H](C)N(Cc2ccc(Cl)cc2Oc2ccc(-c3cnc(CN(C)C)n3C)cc2)C(=O)CCC(=O)N(C)CCNC(=O)C[C@H](Cc2ccc(Cl)cc2)N(C)C1=O] | Compound 150 (Example 2.1) | Analytical method 7 t_{R} = 0.93 min [M + H]⁺ = 891.4 |
| 145 | 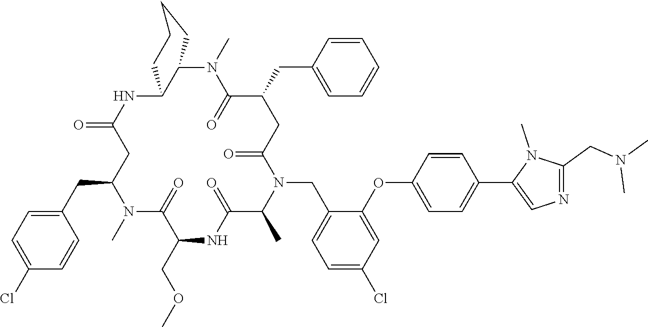 | Compound 150 (Example 2.1) | Analytical method 6 t_{R} = 2.31 min [M + H]⁺ = 1035.5 |
| 149 | 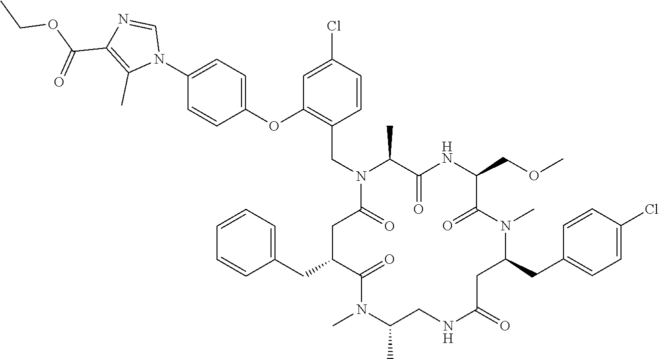 [SMILES: CCOC(=O)c1ncn(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)c1C] | Compound 150 (Example 2.1) | Analytical method 4 t_{R} = 2.87 min [M + 2H]²⁺ = 506.7 |
| 152 | 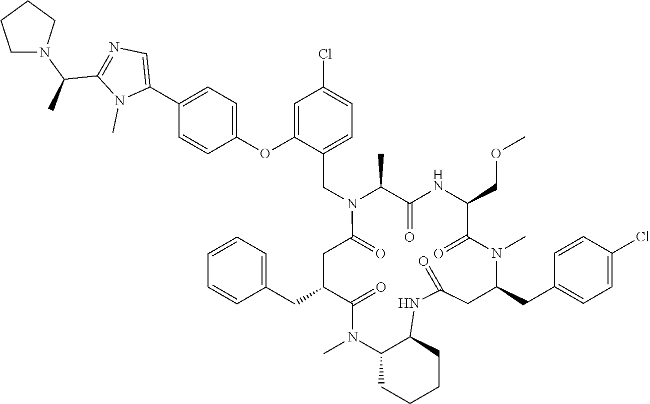 [SMILES: COC[C@@H]1NC(=O)[C@H](C)N(Cc2ccc(Cl)cc2Oc2ccc(-c3cnc([C@@H](C)N4CCCC4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 2 t_{R} = 3.27 min [M + H]⁺ = 1076.0 |
| 155 | 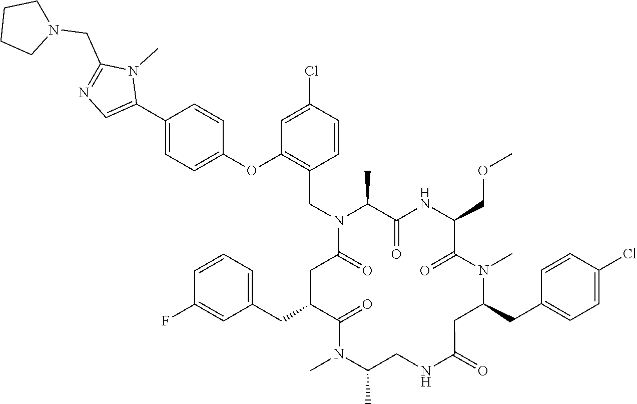 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2cccc(F)c2)C(=O)N(C)[C@@H](C)CNC(=O)C[C@H](Cc2ccc(Cl)cc2)N(C)C1=O] | Compound 150 (Example 2.1) | Analytical method 9 t_{R} = 4.75 min [M + H]⁺ = 1039.4 |
| 156 | 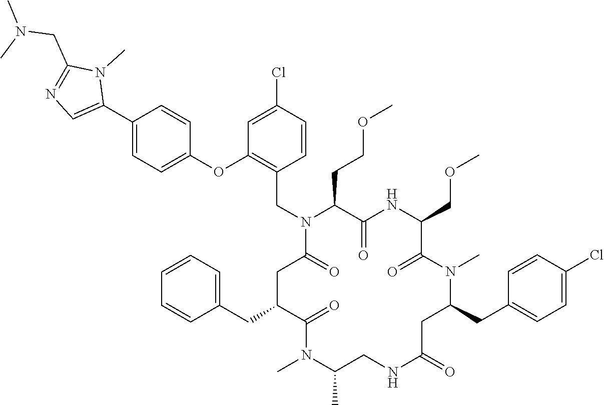 [SMILES: COCC[C@H]1C(=O)N[C@@H](COC)C(=O)N(C)[C@@H](Cc2ccc(Cl)cc2)CC(=O)NC[C@H](C)N(C)C(=O)[C@H](Cc2ccccc2)CC(=O)N1Cc1ccc(Cl)cc1Oc1ccc(-c2cnc(CN(C)C)n2C)cc1] | Compound 150 (Example 2.1)/ Polymer-bound pentamers were synthesized with an N-terminal Boc group. After cleavage from resin with HFIP, Boc was removed by treatment with TFA. | Analytical method 2 t_{R} = 2.91 min [M + H]⁺ = 1039.6 |
| 157 | 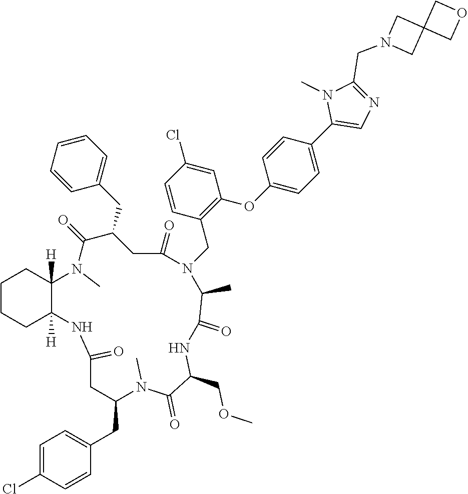 [SMILES: COC[C@@H]1NC(=O)[C@H](C)N(Cc2ccc(Cl)cc2Oc2ccc(-c3cnc(CN4CC5(COC5)C4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 4 t_{R} = 2.13 min [M + H]⁺ = 1089.9 |
| 163 | 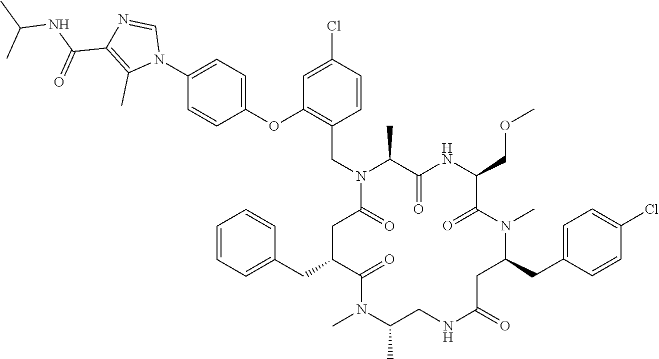 [SMILES: COC[C@@H]1NC(=O)[C@H](C)N(Cc2ccc(Cl)cc2Oc2ccc(-n3cnc(C(=O)NC(C)C)c3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 4 t_{R} = 2.91 min [M + H]⁺ = 1023.2 |
| 164 | 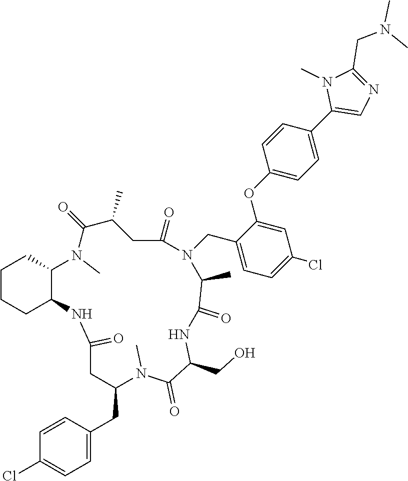 [SMILES: C[C@@H]1CC(=O)N(Cc2ccc(Cl)cc2Oc2ccc(-c3cnc(CN(C)C)n3C)cc2)[C@@H](C)C(=O)N[C@@H](CO)C(=O)N(C)[C@@H](Cc2ccc(Cl)cc2)CC(=O)N[C@H]2CCCC[C@@H]2N(C)C1=O] | Compound 150 (Example 2.1) | Analytical method 6 t_{R} = 2.09 min [M + H]⁺ = 945.4 |
| 165 | 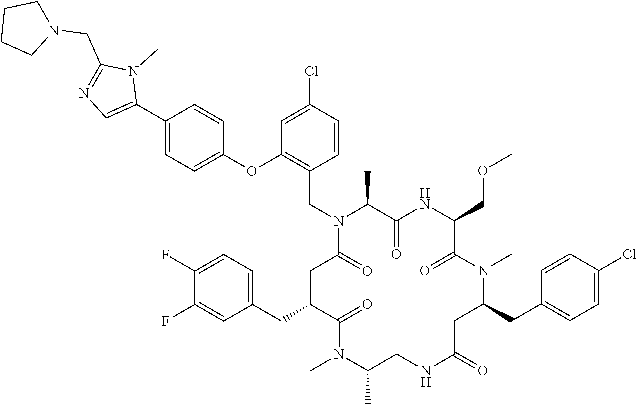 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccc(F)c(F)c2)C(=O)N(C)[C@@H](C)CNC(=O)C[C@H](Cc2ccc(Cl)cc2)N(C)C1=O] | Compound 150 (Example 2.1) | Analytical method 9 t_{R} = 4.73 min [M + H]⁺ = 1057.4 |
| 166 | 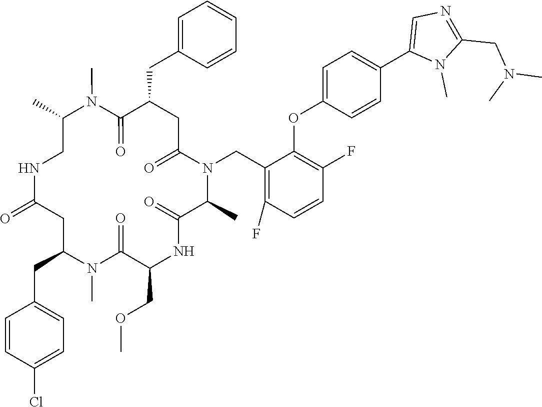 [SMILES: COC[C@@H]1NC(=O)[C@H](C)N(Cc2c(F)ccc(F)c2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 7 t_{R} = 1.00 min [M + H]⁺ = 997.4 |
| 168 | 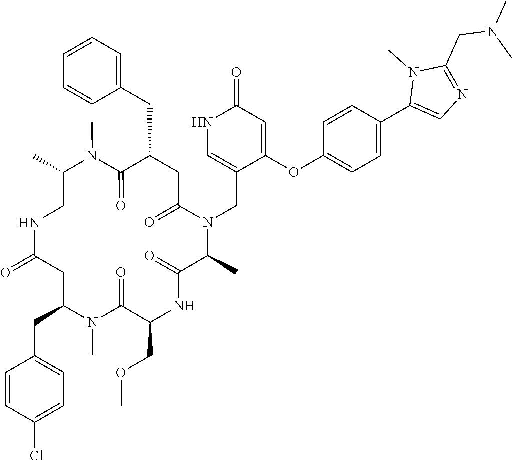 [SMILES: COC[C@@H]1NC(=O)[C@H](C)N(Cc2c[nH]c(=O)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 2 t_{R} = 2.17 min [M + H]⁺ = 978.4 |
| 174 | 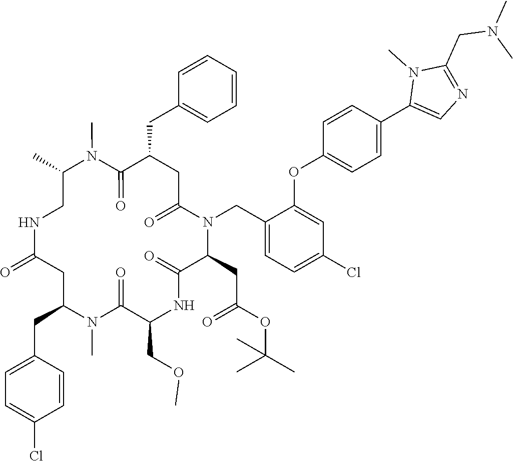 [SMILES: COC[C@@H]1NC(=O)[C@H](CC(=O)OC(C)(C)C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 3 t_{R} = 1.20 min [M + H]⁺ = 1095.5 |
| 178 | 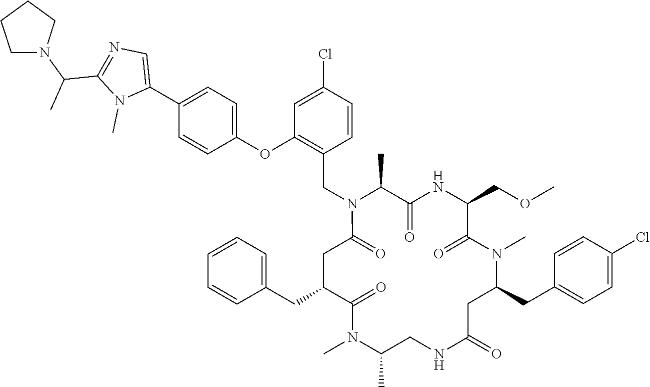 [SMILES: COC[C@@H]1NC(=O)[C@H](C)N(Cc2ccc(Cl)cc2Oc2ccc(-c3cnc(C(C)N4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 9 t_{R} = 4.88 min [M + H]⁺ = 1035.5 |
| 179 | 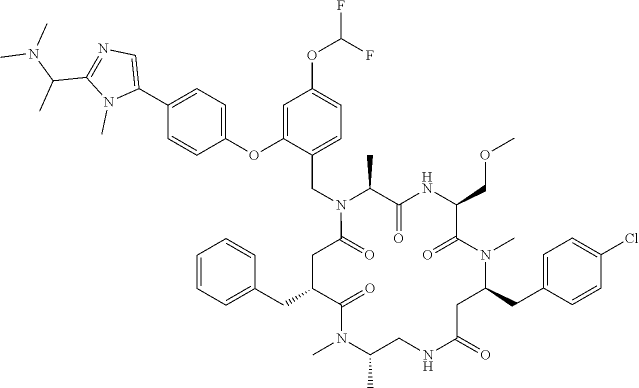 | Compound 150 (Example 2.1) | Analytical method 7 t_{R} = 1.02 min [M + H]⁺ = 1027.7 |
| 184 | 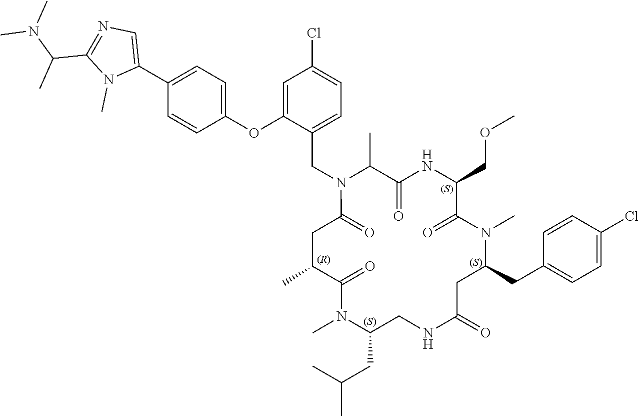 | Compound 150 (Example 2.1) | Analytical method 7 t_{R} = 1.00 min [M + H]⁺ = 961.3 |
| 188 | 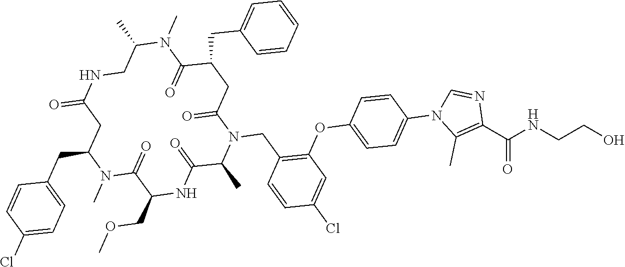 [SMILES: COC[C@@H]1NC(=O)[C@H](C)N(Cc2ccc(Cl)cc2Oc2ccc(-n3cnc(C(=O)NCCO)c3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 4 t_{R} = 2.53 min [M + H]⁺ = 513.0 |
| 189 | 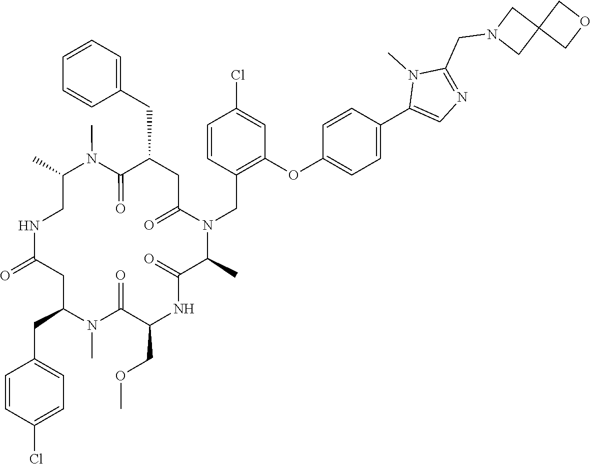 [SMILES: COC[C@@H]1NC(=O)[C@H](C)N(Cc2ccc(Cl)cc2Oc2ccc(-c3cnc(CN4CC5(COC5)C4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 2 t_{R} = 2.63 min [M + H]⁺ = 1049.7 |
| 190 | 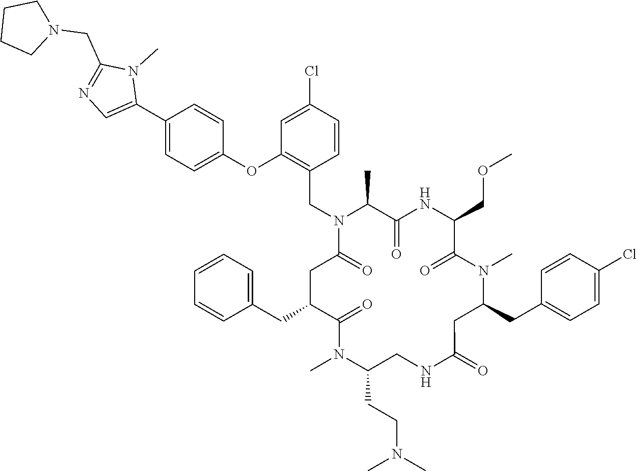 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](CCN(C)C)CNC(=O)C[C@H](Cc2ccc(Cl)cc2)N(C)C1=O] | Compound 150 (Example 2.1) | Analytical method 9 t_{R} = 3.70 min [M + H]⁺ = 1078.5 |
| 194 | 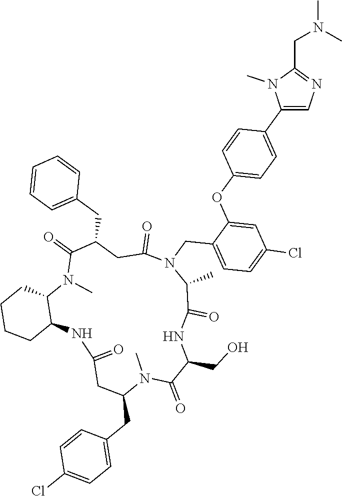 [SMILES: C[C@@H]1C(=O)N[C@@H](CO)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1] | Compound 150 (Example 2.1) | Analytical method 6 t_{R} = 2.39 min [M + H]⁺ = 1021.5 |
| 197 | 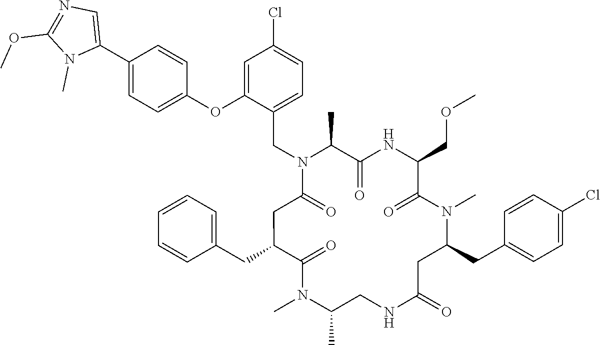 [SMILES: COC[C@@H]1NC(=O)[C@H](C)N(Cc2ccc(Cl)cc2Oc2ccc(-c3cnc(OC)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 3 t_{R} = 1.25 min [M + H]⁺ = 968.4 |
| 199 | 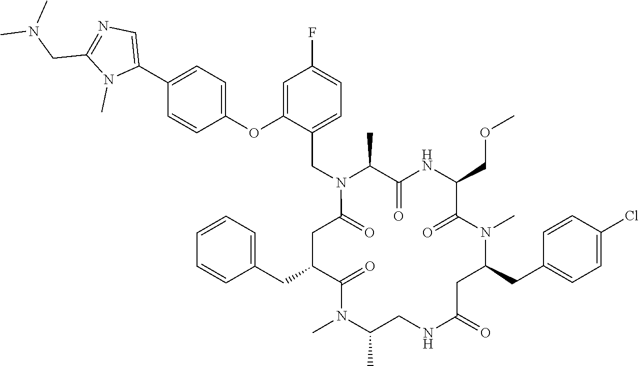 [SMILES: COC[C@@H]1NC(=O)[C@H](C)N(Cc2ccc(F)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 3 t_{R} = 1.02 min [M + H]⁺ = 979.4 |
| 203 | 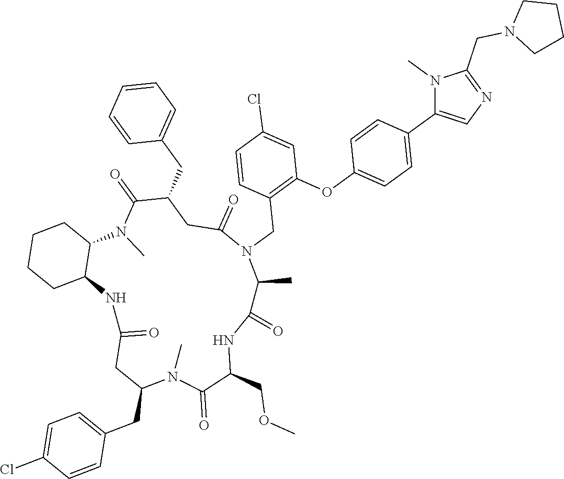 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 3 t_{R} = 1.10 min [M + H]⁺ = 1061.5 |
| 205 | 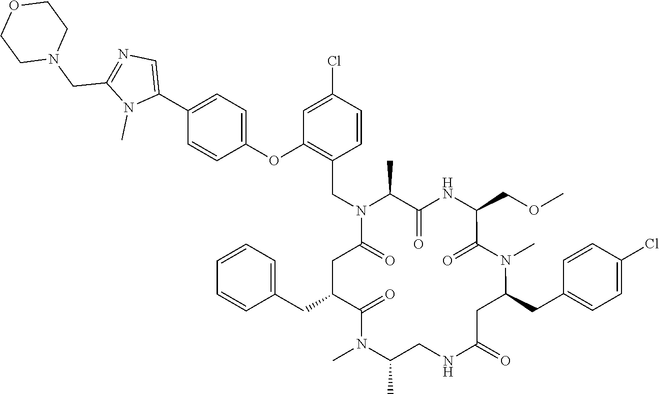 [SMILES: COC[C@@H]1NC(=O)[C@H](C)N(Cc2ccc(Cl)cc2Oc2ccc(-c3cnc(CN4CCOCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 9 t_{R} = 4.83 min [M + H]⁺ = 1037.4 |
| 206 | 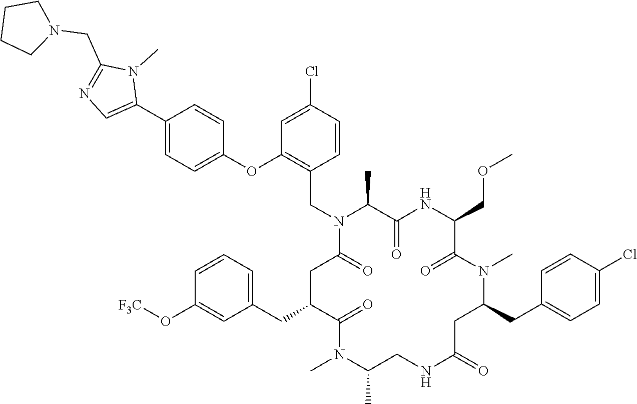 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2cccc(OC(F)(F)F)c2)C(=O)N(C)[C@@H](C)CNC(=O)C[C@H](Cc2ccc(Cl)cc2)N(C)C1=O] | Compound 150 (Example 2.1) | Analytical method 9 t_{R} = 5.13 min [M + H]⁺ = 1105.4 |
| 207A | 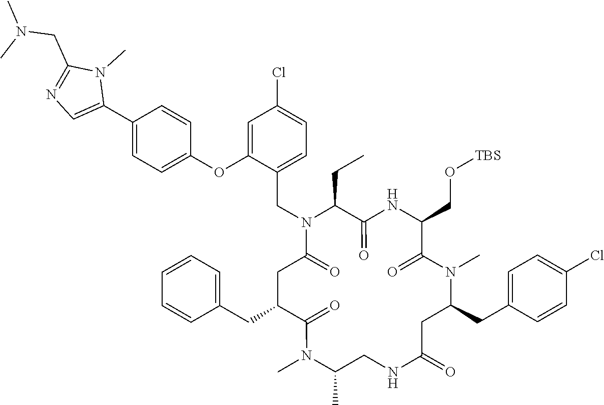 | Compound 150 (Example 2.1) | Analytical method 5 t_{R} = 1.22 min [M + H]⁺ = 1109.6 |
| 208 | 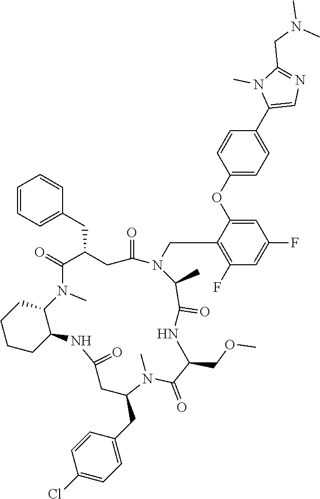 [SMILES: COC[C@@H]1NC(=O)[C@H](C)N(Cc2c(F)cc(F)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 3 t_{R} = 1.07 min [M + H]⁺ = 1037.5 |
| 211 | 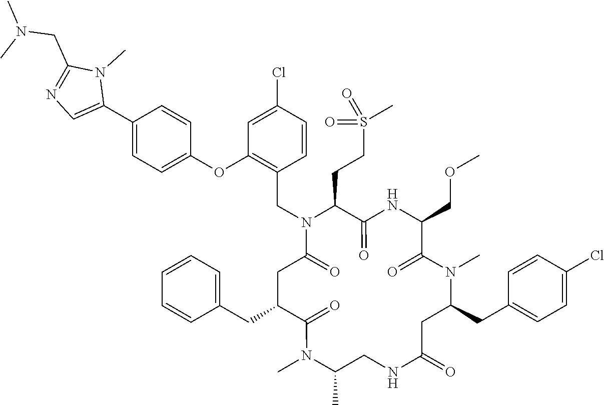 [SMILES: COC[C@@H]1NC(=O)[C@H](CCS(C)(=O)=O)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1)/ Polymer-bound pentamers were synthesized with an N-terminal Boc group. After cleavage from resin with HFIP, Boc was removed by treatment with TFA. | Analytical method 4 t_{R} = 2.00 min [M − H]⁻ = 1085.8 |
| 216 | 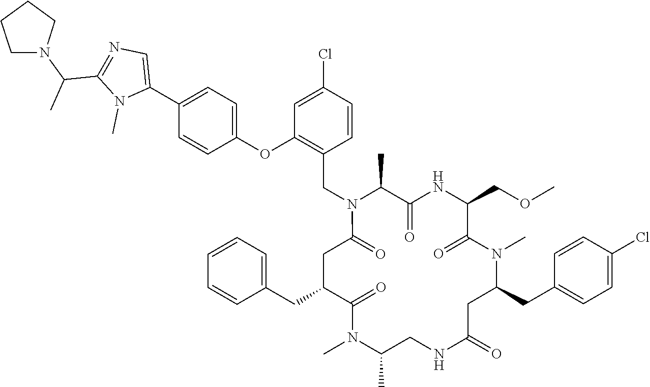 [SMILES: COC[C@@H]1NC(=O)[C@H](C)N(Cc2ccc(Cl)cc2Oc2ccc(-c3cnc(C(C)N4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 9 t_{R} = 5.06 min [M + H]⁺ = 1035.5 |
| 220A | 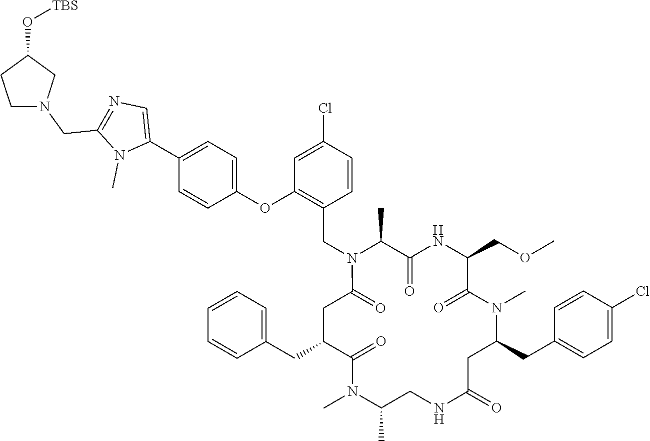 | Compound 150 (Example 2.1) | Analytical method 10 t_{R} = 1.36 min [M + H]⁺ = 1151.7 |
| 221 | 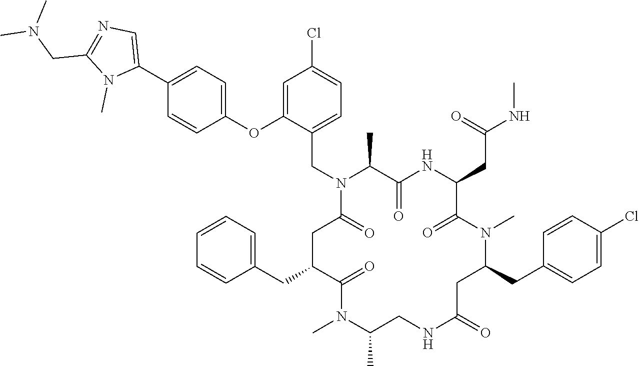 [SMILES: CNC(=O)C[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 7 t_{R} = 0.89 min [M + H]⁺ = 1022.7 |
| 222 | 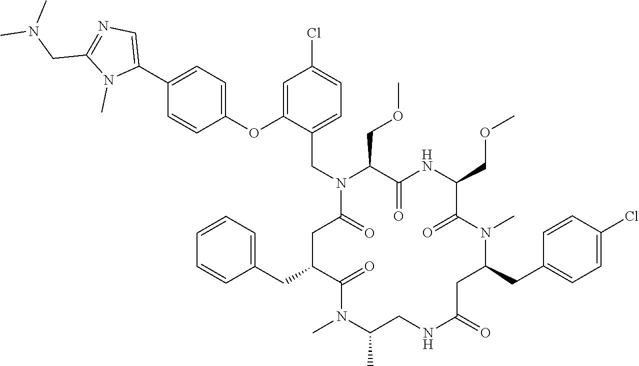 | Compound 150 (Example 2.1) | Analytical method 6 t_{R} = 2.31 min [M + H]⁺ = 998.4 |
| 225 | 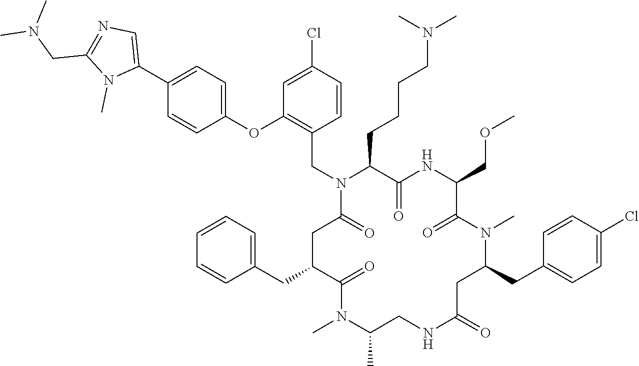 [SMILES: COC[C@@H]1NC(=O)[C@H](CCCCN(C)C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 2 t_{R} = 3.14 min [M + 2H]²⁺ = 540.7 |
| 228 | 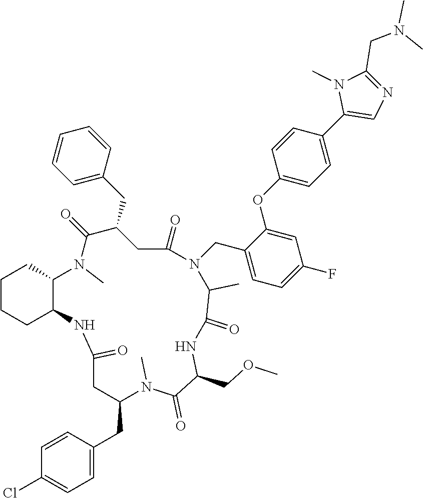 [SMILES: COC[C@@H]1NC(=O)C(C)N(Cc2ccc(F)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 7 t_{R} = 1.03 min [M + H]⁺ = 1019.4 |
| 229 | 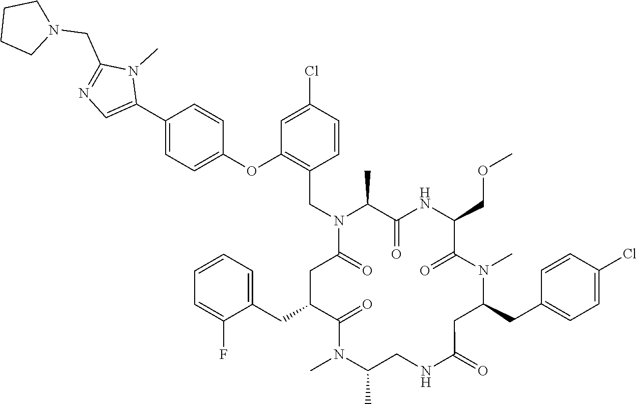 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2F)C(=O)N(C)[C@@H](C)CNC(=O)C[C@H](Cc2ccc(Cl)cc2)N(C)C1=O] | Compound 150 (Example 2.1) | Analytical method 9 t_{R} = 4.74 min [M + H]⁺ = 1039.4 |
| 230 | 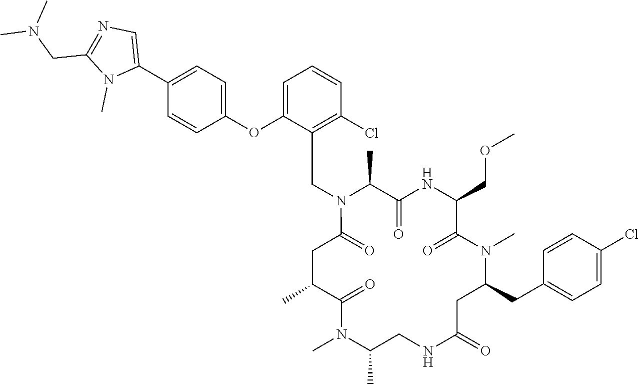 [SMILES: COC[C@@H]1NC(=O)[C@H](C)N(Cc2c(Cl)cccc2Oc2ccc(-c3cnc(CN(C)C)n3C)cc2)C(=O)C[C@@H](C)C(=O)N(C)[C@@H](C)CNC(=O)C[C@H](Cc2ccc(Cl)cc2)N(C)C1=O] | Compound 150 (Example 2.1) | Analytical method 7 t_{R} = 1.04 min [M + H]⁺ = 919.0 |
| 232 | 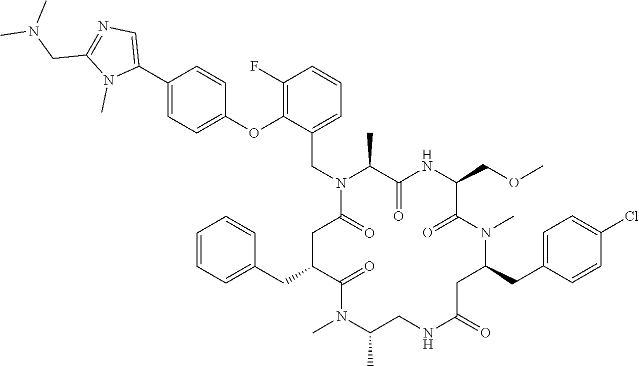 [SMILES: COC[C@@H]1NC(=O)[C@H](C)N(Cc2cccc(F)c2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 7 t_{R} = 0.99 min [M + H]⁺ = 979.4 |
| 234 | 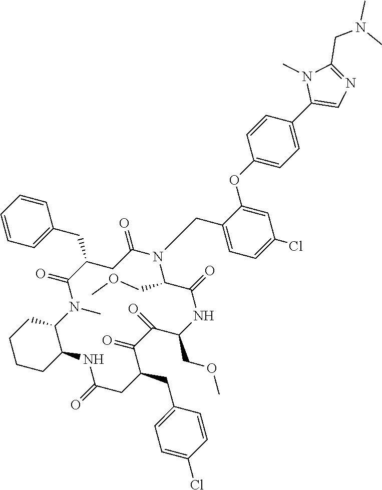 [SMILES: COC[C@@H]1NC(=O)[C@H](COC)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)C(=O)C1=O] | Compound 150 (Example 2.1) | Analytical method 3 t_{R} = 1.12 min [M + H]⁺ = 1065.5 |
| 236 | 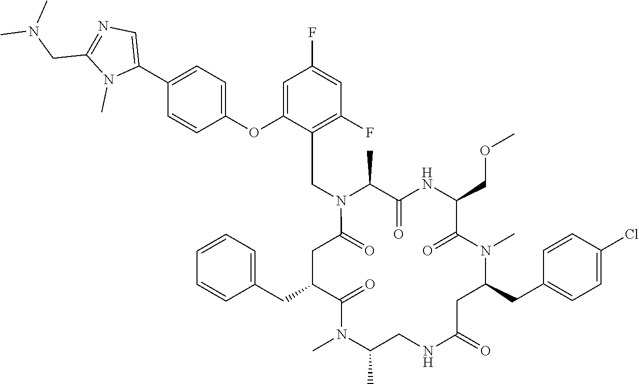 [SMILES: COC[C@@H]1NC(=O)[C@H](C)N(Cc2c(F)cc(F)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 7 t_{R} = 0.96 min [M + H]⁺ = 997.8 |
| 237 | 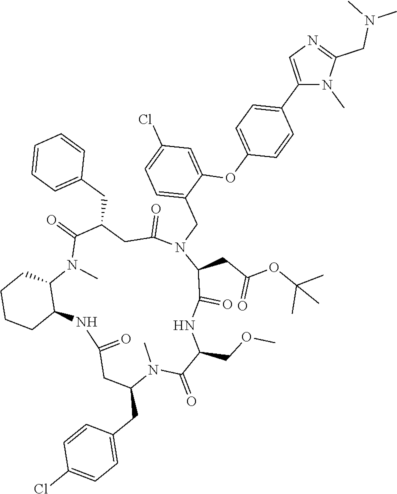 [SMILES: COC[C@@H]1NC(=O)[C@H](CC(=O)OC(C)(C)C)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 6 t_{R} = 2.55 min [M + H]⁺ = 1135.5 |
| 240 | 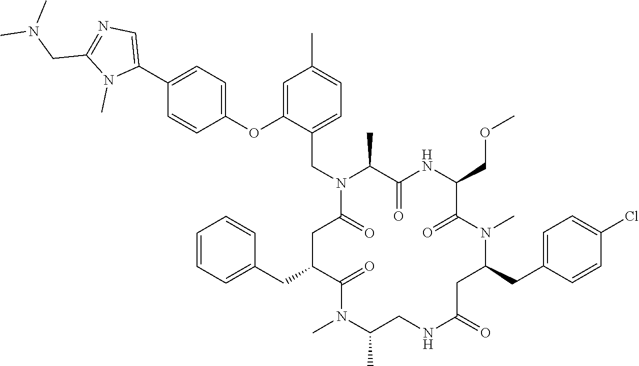 [SMILES: COC[C@@H]1NC(=O)[C@H](C)N(Cc2ccc(C)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 7 t_{R} = 1.02 min [M + H]⁺ = 975.4 |
| 243 | 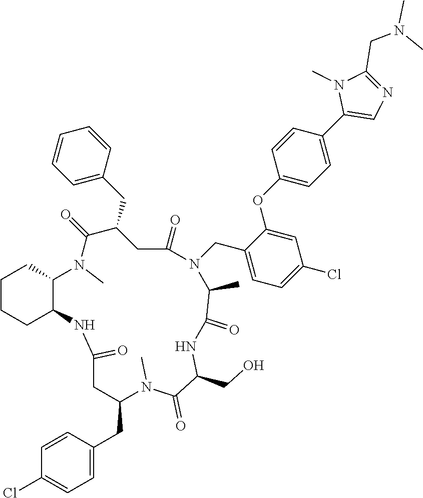 [SMILES: C[C@H]1C(=O)N[C@@H](CO)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1] | Compound 150 (Example 2.1)/ TBS-protected serine D11 was used for pentamer synthesis. The TBS group was removed during purification under acidic conditions. | Analytical method 6 t_{R} = 2.32 min [M + H]⁺ = 1021.5 |
| 255 | 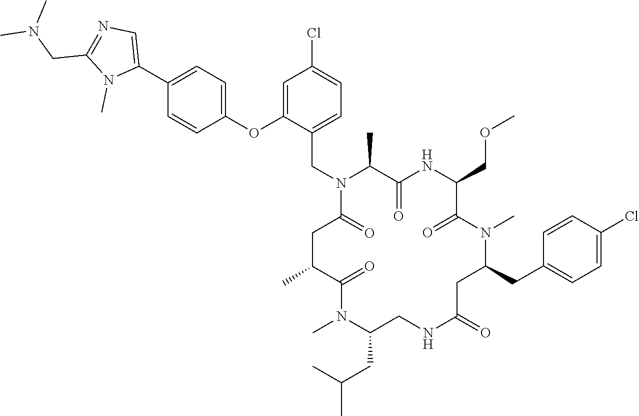 [SMILES: COC[C@@H]1NC(=O)[C@H](C)N(Cc2ccc(Cl)cc2Oc2ccc(-c3cnc(CN(C)C)n3C)cc2)C(=O)C[C@@H](C)C(=O)N(C)[C@@H](CC(C)C)CNC(=O)C[C@H](Cc2ccc(Cl)cc2)N(C)C1=O] | Compound 150 (Example 2.1) | Analytical method 7 t_{R} = 1.06 min [M + H]⁺ = 961.3 |
| 256 | 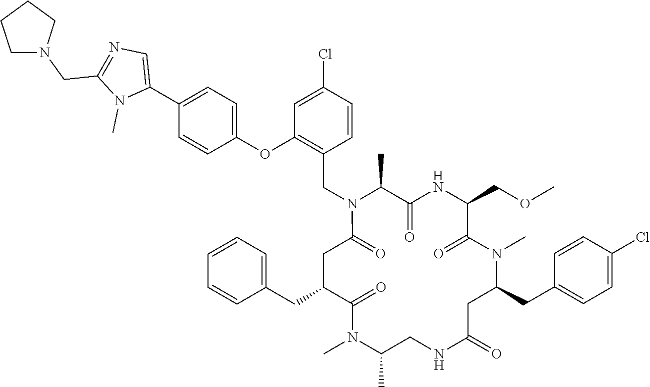 | Compound 150 (Example 2.1) | Analytical method 9 t_{R} = 4.88 min [M + H]⁺ = 1007.4 |
| 257 | 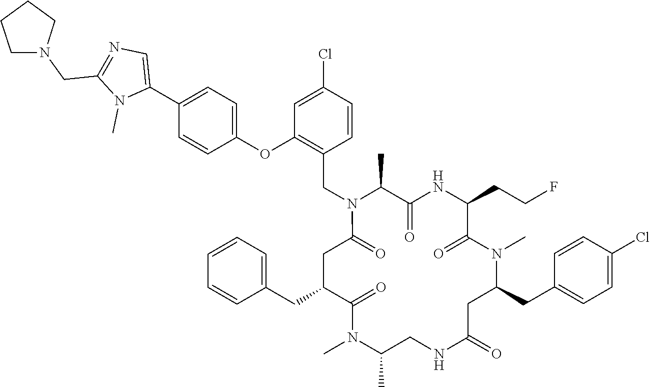 [SMILES: C[C@H]1CNC(=O)C[C@H](Cc2ccc(Cl)cc2)N(C)C(=O)[C@H](CCF)NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N1C] | Compound 150 (Example 2.1) | Analytical method 9 t_{R} = 4.70 min [M + H]⁺ = 1023.4 |
| 259 | 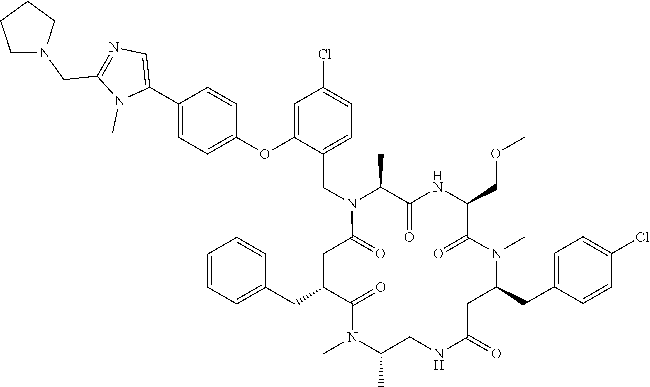 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 5 t_{R} = 1.26 min [M + H]⁺ = 1022.0 |
| 260A | 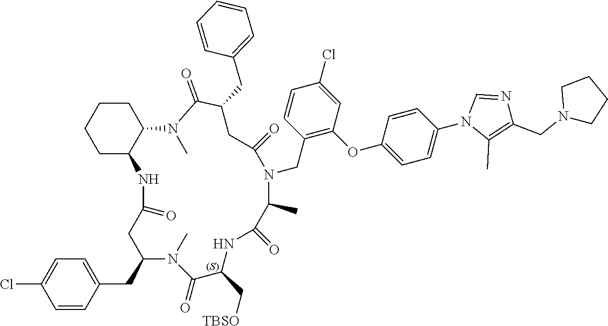 | Compound 150 (Example 2.1) | Analytical method 5 t_{R} = 1.67 min [M + H]⁺ = 1160.6 |
| 262 | 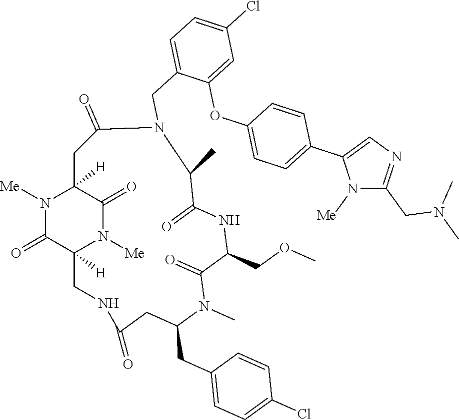 [SMILES: COC[C@@H]1NC(=O)[C@H](C)N(Cc2ccc(Cl)cc2Oc2ccc(-c3cnc(CN(C)C)n3C)cc2)C(=O)C[C@H]2C(=O)N(C)[C@@H](CNC(=O)C[C@H](Cc3ccc(Cl)cc3)N(C)C1=O)C(=O)N2C] | Compound 150 (Example 2.1) | Analytical method 4 t_{R} = 1.73 min [M + H]⁺ = 946.3 |
| 266 | 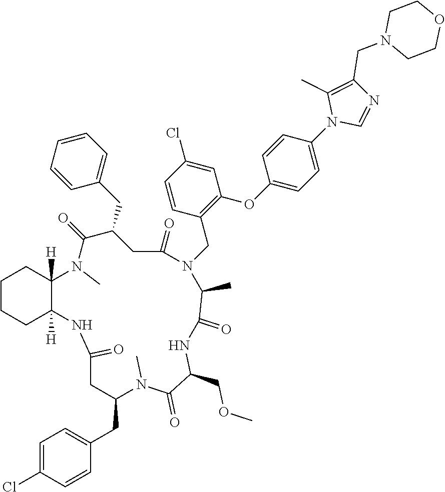 [SMILES: COC[C@@H]1NC(=O)[C@H](C)N(Cc2ccc(Cl)cc2Oc2ccc(-n3cnc(CN4CCOCC4)c3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 4 t_{R} = 2.18 min [M + H]⁺ = 1077.5 |
| 268 | 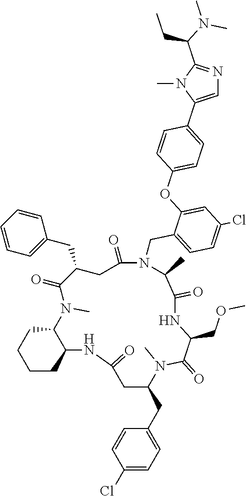 [SMILES: CC[C@H](c1ncc(-c2ccc(Oc3cc(Cl)ccc3CN3C(=O)C[C@@H](Cc4ccccc4)C(=O)N(C)[C@H]4CCCC[C@@H]4NC(=O)C[C@H](Cc4ccc(Cl)cc4)N(C)C(=O)[C@H](COC)NC(=O)[C@@H]3C)cc2)n1C)N(C)C] | Compound 150 (Example 2.1) | Analytical method 2 t_{R} = 3.12 min [M + H]⁺ = 1064.2 |
| 271 | 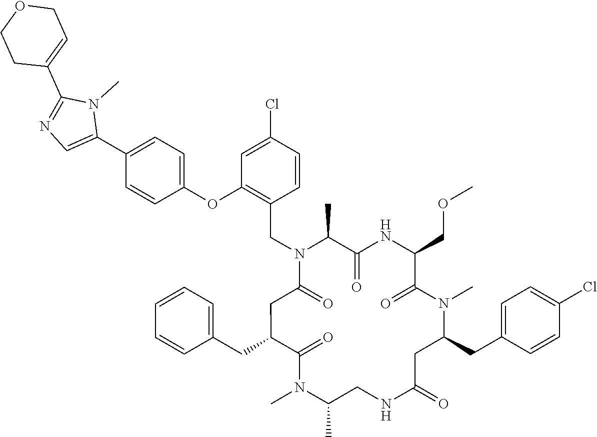 [SMILES: COC[C@@H]1NC(=O)[C@H](C)N(Cc2ccc(Cl)cc2Oc2ccc(-c3cnc(C4=CCOCC4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 9 t_{R} = 5.05 min [M + H]⁺ = 1020.4 |
| 273 | 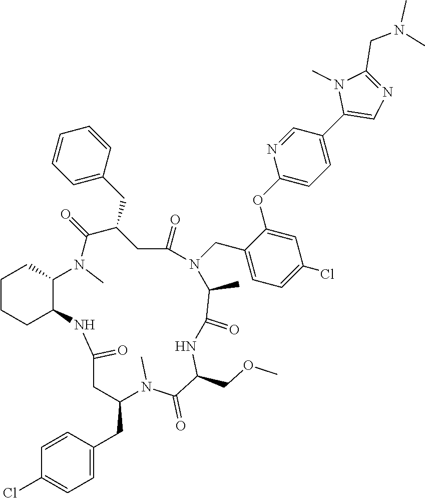 [SMILES: COC[C@@H]1NC(=O)[C@H](C)N(Cc2ccc(Cl)cc2Oc2ccc(-c3cnc(CN(C)C)n3C)cn2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 5 t_{R} = 1.18 min [M + H]⁺ = 1036.5 |
| 278A | 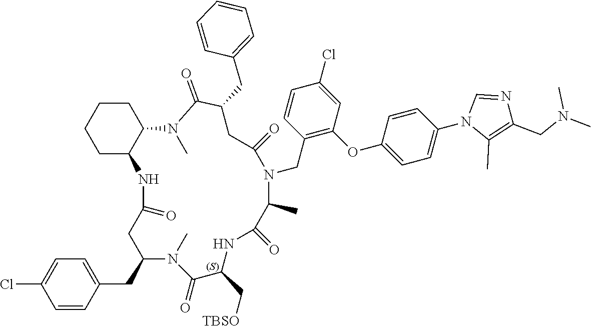 | Compound 150 (Example 2.1) | Analytical method 5 t_{R} = 1.49 min [M + H]⁺ = 1135.4 |
| 280 | 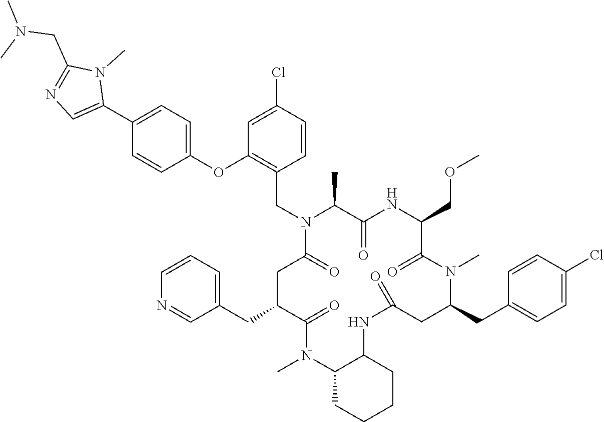 | Compound 150 (Example 2.1) | Analytical method 14 t_{R} = 3.49 min [M + H]⁺ = 1036.5 |
| 281 | 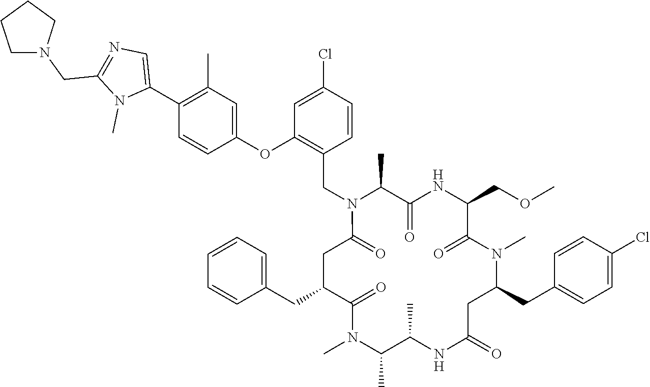 | Compound 150 (Example 2.1) | Analytical method 13 t_{R} = 4.43 min [M + H]⁺ = 1035.5 |
| 286 | 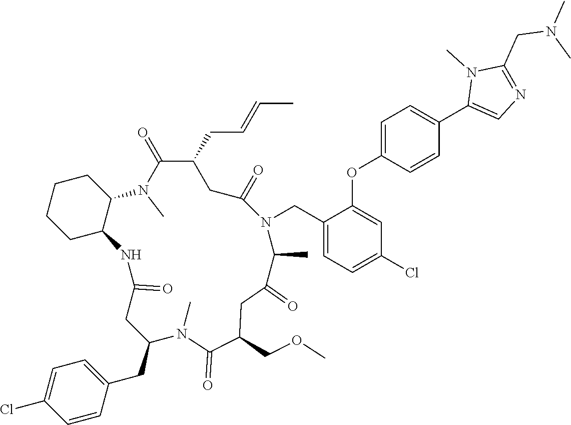 [SMILES: C/C=C/C[C@@H]1CC(=O)N(Cc2ccc(Cl)cc2Oc2ccc(-c3cnc(CN(C)C)n3C)cc2)[C@@H](C)C(=O)C[C@@H](COC)C(=O)N(C)[C@@H](Cc2ccc(Cl)cc2)CC(=O)N[C@H]2CCCC[C@@H]2N(C)C1=O] | Compound 150 (Example 2.1) | Analytical method 7 t_{R} = 1.04 min [M + H]⁺ = 999.6 |
| 287 | 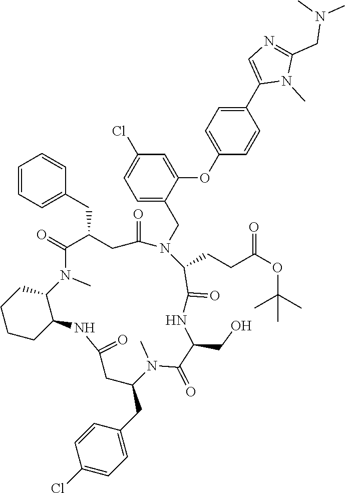 [SMILES: CN(C)Cc1ncc(-c2ccc(Oc3cc(Cl)ccc3CN3C(=O)C[C@@H](Cc4ccccc4)C(=O)N(C)[C@H]4CCCC[C@@H]4NC(=O)C[C@H](Cc4ccc(Cl)cc4)N(C)C(=O)[C@H](CO)NC(=O)[C@H]3CCC(=O)OC(C)(C)C)cc2)n1C] | Compound 150 (Example 2.1) | Analytical method 3 t_{R} = 1.14 min [M + H]⁺ = 1135.5 |
| 288 | 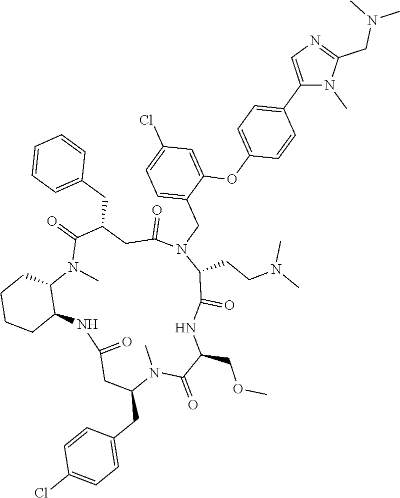 [SMILES: COC[C@@H]1NC(=O)[C@@H](CCN(C)C)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 7 t_{R} = 0.82 min [M + H]⁺ = 1091.6 |
| 289 | 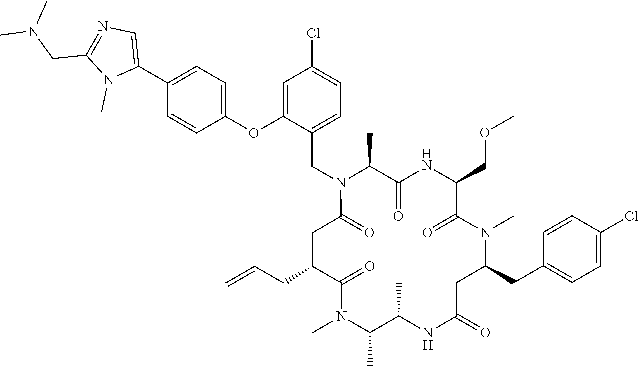 [SMILES: C=CC[C@@H]1CC(=O)N(Cc2ccc(Cl)cc2Oc2ccc(-c3cnc(CN(C)C)n3C)cc2)[C@@H](C)C(=O)N[C@@H](COC)C(=O)N(C)[C@@H](Cc2ccc(Cl)cc2)CC(=O)N[C@@H](C)[C@H](C)N(C)C1=O] | Compound 150 (Example 2.1) | Analytical method 7 t_{R} = 0.96 min [M + H]⁺ = 959.3 |
| 291 | 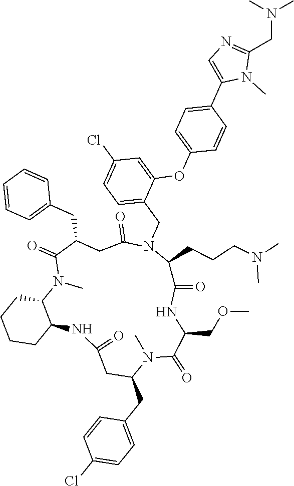 [SMILES: COC[C@@H]1NC(=O)[C@H](CCCN(C)C)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 3 t_{R} = 0.90 min [M + H]⁺ = 1106.5 |
| 292 | 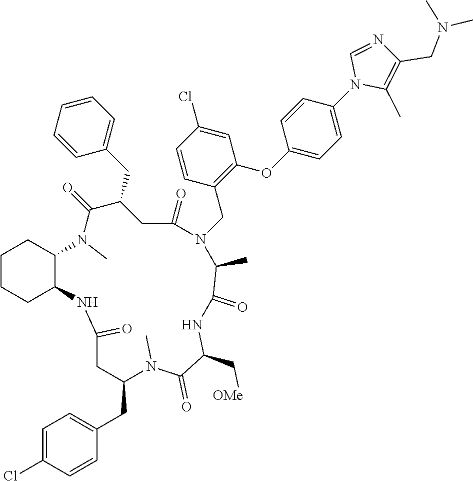 [SMILES: COC[C@@H]1NC(=O)[C@H](C)N(Cc2ccc(Cl)cc2Oc2ccc(-n3cnc(CN(C)C)c3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 150 (Example 2.1) | Analytical method 3 t_{R} = 1.10 min [M + H]⁺ = 1035.5 |
| 298 | 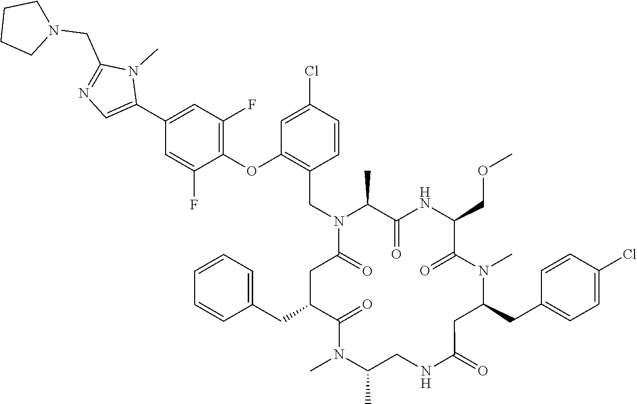 [SMILES: COC[C@@H]1NC(=O)[C@H](C)N(Cc2ccc(Cl)cc2Oc2c(F)cc(-c3cnc(CN4CCCC4)n3C)cc2F)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.43 min [M + H]⁺ = 1057.4 |
| 299 | 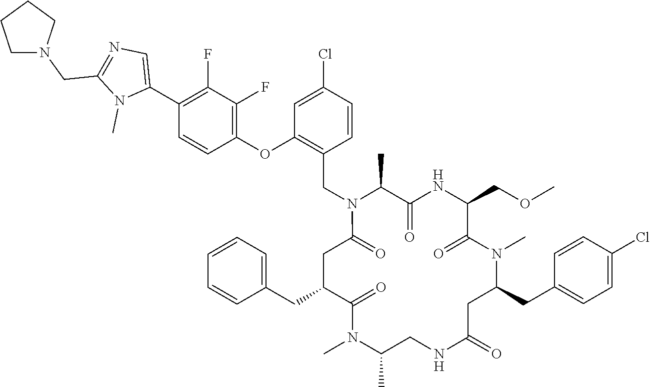 [SMILES: COC[C@@H]1NC(=O)[C@H](C)N(Cc2ccc(Cl)cc2Oc2ccc(-c3cnc(CN4CCCC4)n3C)c(F)c2F)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.50 min [M + H]⁺ = 1057.4 |
| 300 | 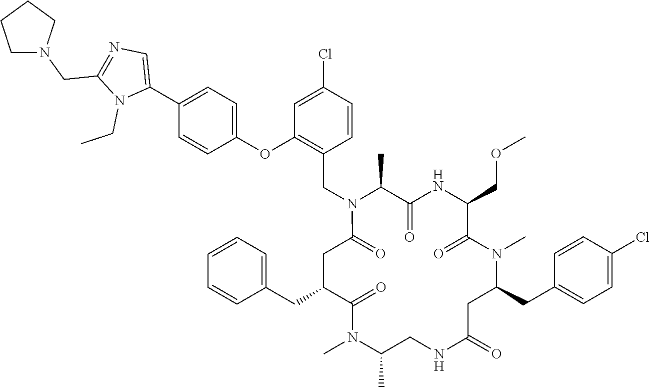 [SMILES: CCn1c(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)cnc1CN1CCCC1] | Compound 150 (Example 2.1) | Analytical method 13 t_{R} = 4.46 min [M + H]⁺ = 1035.5 |
| 301 | 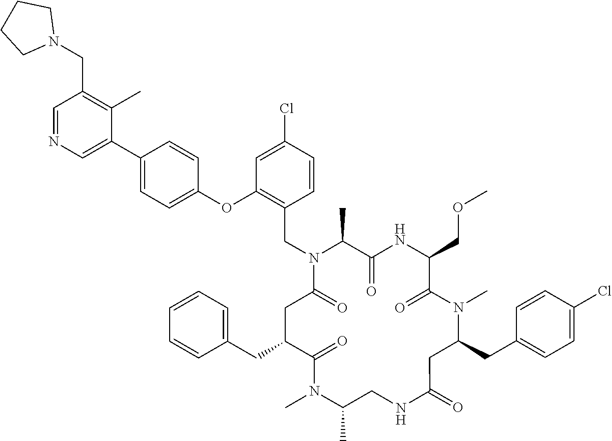 [SMILES: COC[C@@H]1NC(=O)[C@H](C)N(Cc2ccc(Cl)cc2Oc2ccc(-c3cncc(CN4CCCC4)c3C)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.30 min [M + H]⁺ = 1032.5 |
| 302 | 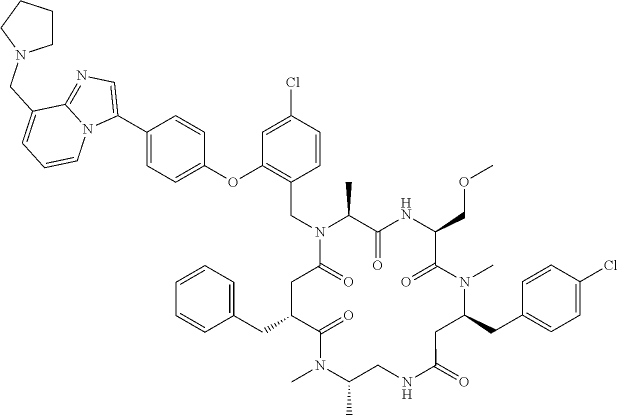 [SMILES: COC[C@@H]1NC(=O)[C@H](C)N(Cc2ccc(Cl)cc2Oc2ccc(-c3cnc4c(CN5CCCC5)cccn34)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.36 min [M + H]⁺ = 1057.4 |
| 303 | 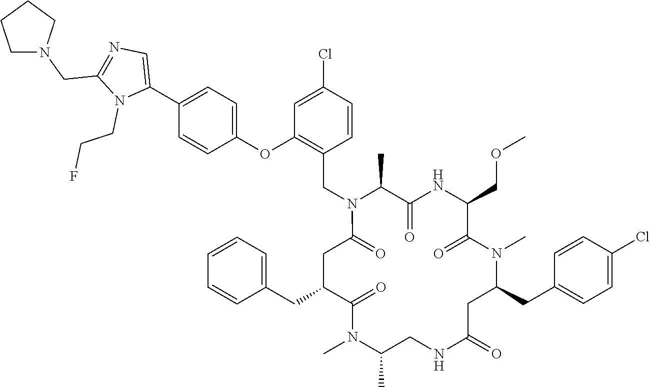 [SMILES: COC[C@@H]1NC(=O)[C@H](C)N(Cc2ccc(Cl)cc2Oc2ccc(-c3cnc(CN4CCCC4)n3CCF)cc2)C(=O)C[C@@H](Cc2ccccc2)C(=O)N(C)[C@@H](C)CNC(=O)C[C@H](Cc2ccc(Cl)cc2)N(C)C1=O] | Compound 150 (Example 2.1) | Analytical method 13 t_{R} = 4.50 min [M + H]⁺ = 1053.4 |

### Example 2.2: Method B—Synthesis of (2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(ethoxymethyl)-2,7,13,14,16-pentamethyl-1,4,7,11,14-pentaaza cyclooctadecane-3,6,10,15,18-pentaone formate (Compound 53)

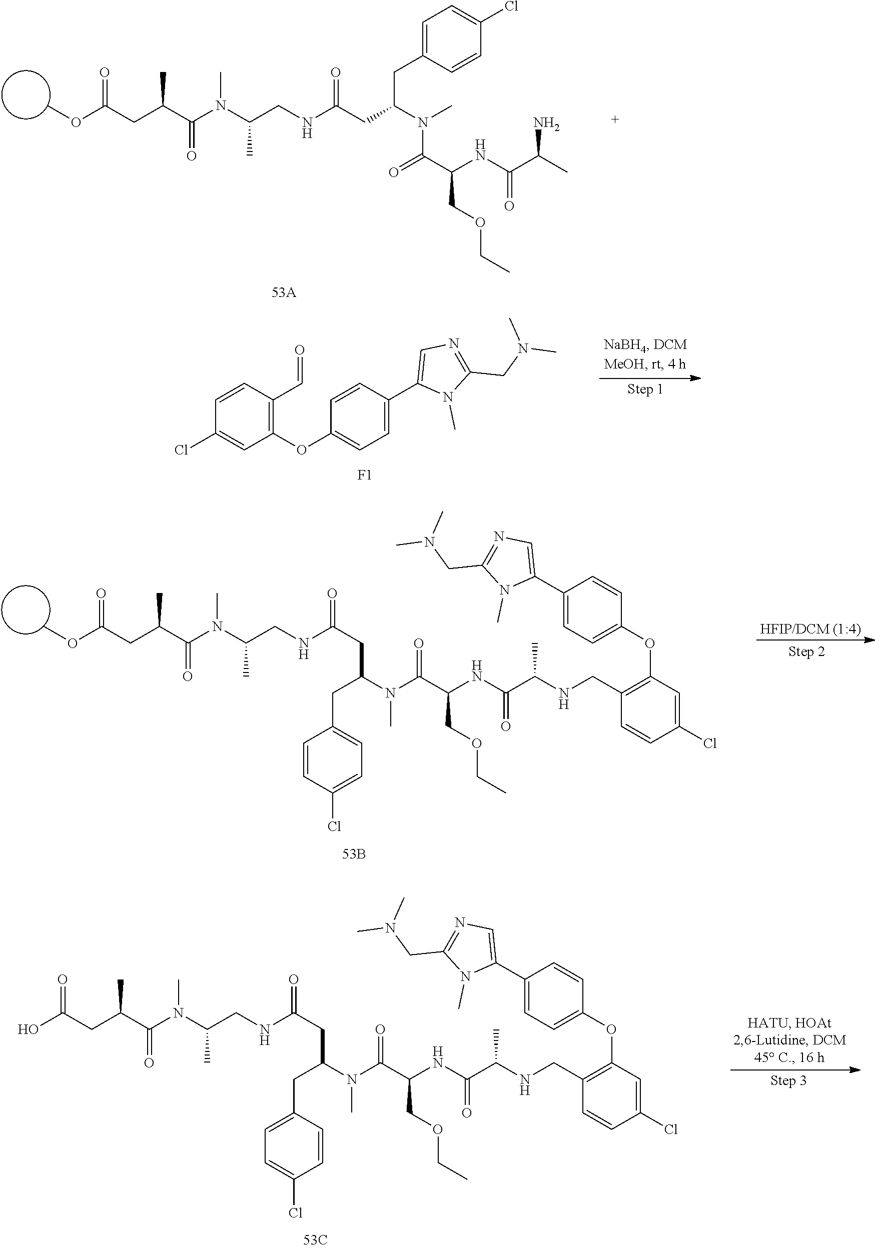

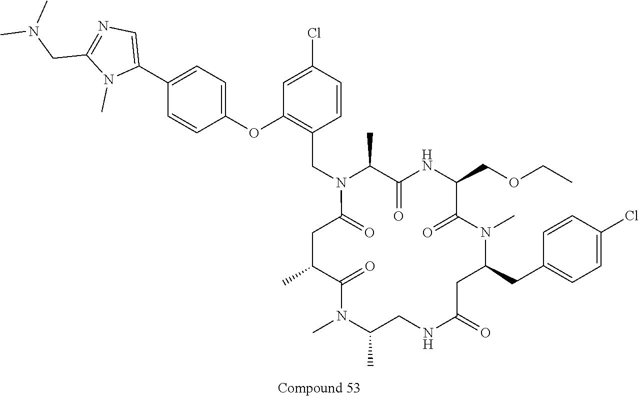 [SMILES: CCOC[C@@H]1NC(=O)[C@H](C)N(Cc2ccc(Cl)cc2Oc2ccc(-c3cnc(CN(C)C)n3C)cc2)C(=O)C[C@@H](C)C(=O)N(C)[C@@H](C)CNC(=O)C[C@H](Cc2ccc(Cl)cc2)N(C)C1=O]

### Step 1. PS-2-Chloro-trityl (3S,6S,9S,14S,17R)-1-(4-chloro-2-(4-(2-((dimethylamino)methyl)-1-methyl-1H-imidazol-5-yl)phenoxy)phenyl)-9-(4-chlorobenzyl)-6-(ethoxymethyl)-3,8,14,15,17-pentamethyl-4,7,11,16-tetraoxo-2,5,8,12,15-pentaazanonadecan-19-oate (53B)

Resin 53A (0.500 g, 0.2 mmol) (prepared according to the procedure used for Compound 150C in Example 2.1) was added to intermediate F1 (0.222 g, 0.600 mmol) in DMF (4 mL). The resulting mixture was shaken at room temperature overnight and then filtered and washed with DMF (anhydrous, 4×10 mL) and DCM (anhydrous, 5×10 mL). To the dried resin, was added DCM/MeOH (75/25) (4 mL) followed by sodium borohydride (0.091 g, 2.400 mmol). The reaction mixture was then slightly agitated for 4 h, filtered, and washed with DMF (3×10 mL) and DCM (3×10 mL). The resin was dried under vacuum to yield 53B (500 mg, crude), which was taken onto the next step without purification.

### Step 2. (3S,6S,9S,14S,17R)-1-(4-chloro-2-(4-(2-((dimethylamino)methyl)-1-methyl-1H-imidazol-5-yl)phenoxy)phenyl)-9-(4-chlorobenzyl)-6-(ethoxymethyl)-3,8,14,15,17-pentamethyl-4,7,11,16-tetraoxo-2,5,8,12,15-pentaazanonadecan-19-oic acid hydrochloride (53C)

TFA/TIPS/H₂O (95:2.5:2.5, 5 mL) was added to resin 53B (500 mg, 0.2 mmol) in a fritted syringe and shaken for 2 h at rt. The reaction mixture was filtered and the filtrate was poured into cold Et₂O/heptane (1:1, 40 mL Falcon tube) and left to stand in an ice-bath for 10 min. Once precipitation was observed, the tube was centrifuged for 5 min at 0° C. at 3000 rpm. The white solid collected at the bottom of the tube, the ether/heptane was poured out, and fresh cold ether was added into the tube. The white solid was broken up with a spatula and centrifuged again. This process was repeated 4 times total. The white solid was then dissolved in THF and converted to HCl salt by adding 4 M HCl in dioxane (2 mL, 8.00 mmol) and stirring the resulting mixture at rt for 2 h. The resulting cloudy precipitate was diluted with ether and centrifuged for 5 min at 0° C. at 3000 rpm. The white solid collected at bottom of tube, the ether was poured out, and fresh ether was added into the tube. The white solid was then broken up with a spatula and the suspension was centrifuged again. The white solid was then left to dry under vacuum for 1 h at rt to yield 53C (130 mg, 63%) as a HCl salt. Analytical method 1; t_{R}=0.99 min; [M+H]⁺=951.8.

### Step 3. (2S,5S,8S,13S,16R)-1-(4-chloro-2-(4-(2-((dimethylamino)methyl)-1-methyl-1H-imidazol-5-yl) phenoxy)benzyl)-8-(4-chlorobenzyl)-5-(ethoxymethyl)-2,7,13,14,16-pentamethyl-1,4,7,11,14-pentaaza cyclooctadecane-3,6,10,15,18-pentaone formate (Compound 53)

To 53C (130 mg, 0.127 mmol) was dissolved in DCM (100 mL) was added HATU (193 mg, 0.507 mmol), 2,6-lutidine (0.443 mL, 3.81 mmol) and HOAt (25.9 mg, 0.190 mmol) and the resulting mixture was stirred for 16 h at 45° C. The reaction mixture was concentrated to dryness and the residue was partitioned between EtOAc (50 mL) and 5% aq. NaHCO₃ (10 mL). The organic layer was washed with 5% aq. NaHCO₃ (2×10 mL) and brine (10 mL), dried over Na₂SO₄, filtered, and concentrated. The obtained light beige oil was purified via reversed-phase chromatography (eluting with ACN/1H₂O with 0.1% Formic acid) to afford the formate salt of Compound 53 (13 mg, 9%) as a white solid. Analytical method 7; t_{R}=1.01 my; [M+H]⁺=933.6.

The Compounds shown in Table 2 were prepared according to the procedure used for Compound 53 (Example 2.2) from the respective intermediates shown in Table 20, Table 22, Table 23, Table 24, and Table 25.

TABLE 2

| Cmd | | Synthesis | |
|---|---|---|---|
| No. | Structure | procedure | LCMS |
| 9 | 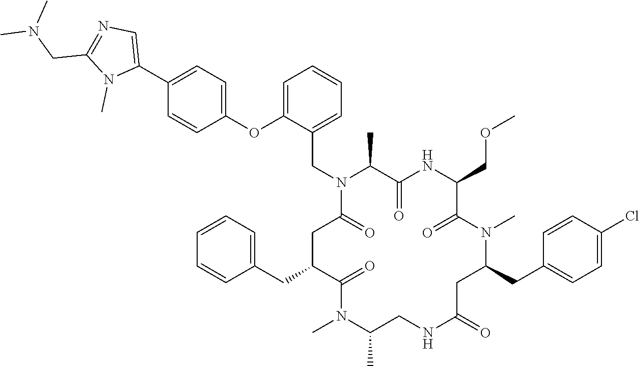 [SMILES: COC[C@@H]1NC(=O)[C@H](C)N(Cc2ccccc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 53 (Example 2.2) | Analytical method 4 t_{R} = 1.93 min [M + H]⁺ = 961.8 |
| 21A | 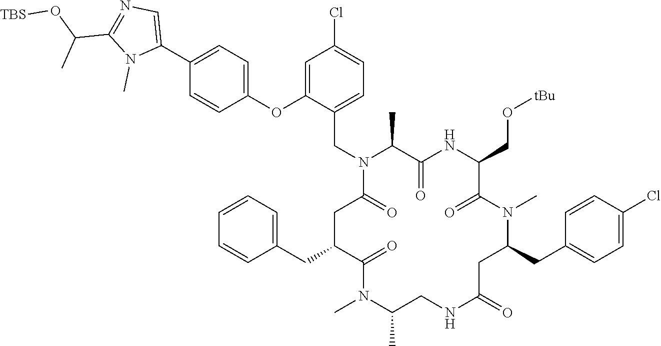 | Compound 53 (Example 2.2) | Analytical method 5 t_{R} = 1.76 min [M − H]⁻ = 1136.4 |
| 37 | 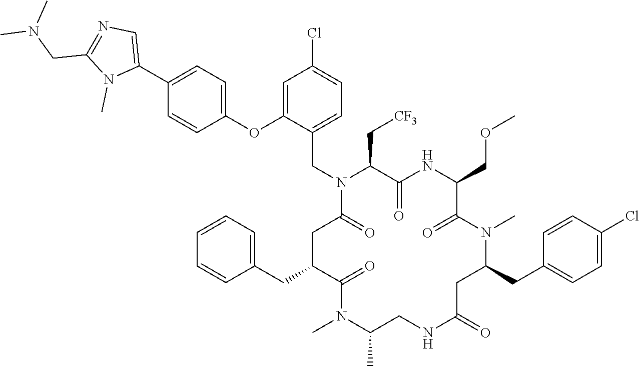 [SMILES: COC[C@@H]1NC(=O)[C@H](CC(F)(F)F)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 53 (Example 2.2) | Analytical method 6 t_{R} = 2.41 min [M + H]⁺ = 1063.4 |
| 39 | 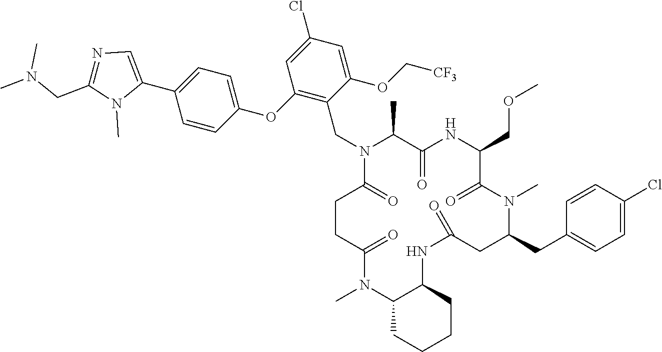 [SMILES: COC[C@@H]1NC(=O)[C@H](C)N(Cc2c(OCC(F)(F)F)cc(Cl)cc2Oc2ccc(-c3cnc(CN(C)C)n3C)cc2)C(=O)CCC(=O)N(C)[C@H]2CCCC[C@@H]2NC(=O)C[C@H](Cc2ccc(Cl)cc2)N(C)C1=O] | Compound 53 (Example 2.2) | Analytical method 4 t_{R} = 2.04 min [M + H]⁺ = 1042.9 |
| 49 | 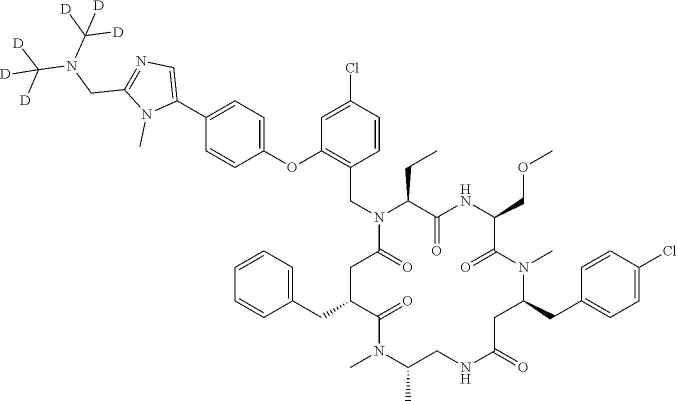 [SMILES: [2H]C([2H])([2H])N(Cc1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3CC)cc2)n1C)C([2H])([2H])[2H]] | Compound 53 (Example 2.2) | Analytical method 7 t_{R} = 0.98 min [M + H]⁺ = 1015.8 |
| 50 | 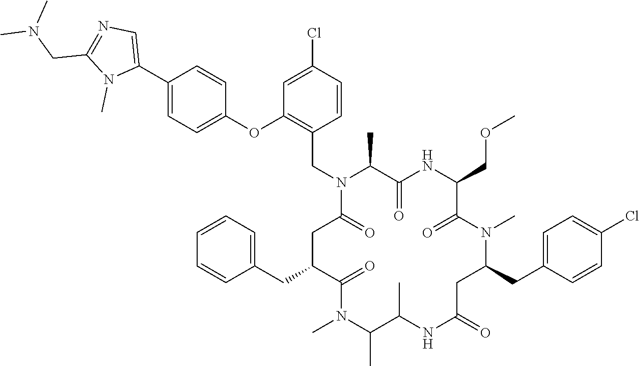 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)C(C)C(C)NC(=O)C[C@H](Cc2ccc(Cl)cc2)N(C)C1=O] | Compound 53 (Example 2.2) | Analytical method 6 t_{R} = 2.30 min [M + H]⁺ = 1009.5 |
| 66A | 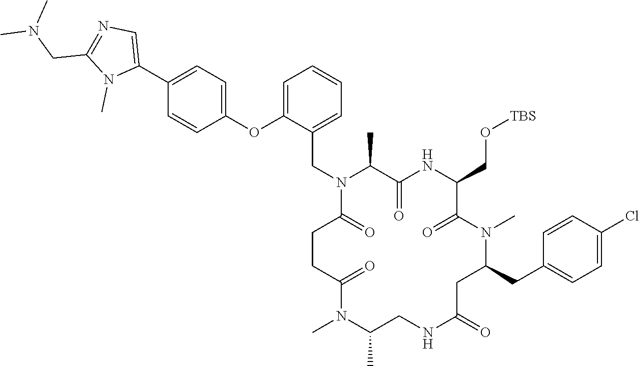 | Compound 53 (Example 2.2) | Analytical method 8 t_{R} = 1.62 min [M + H]⁺ = 971.5 |
| 69 | 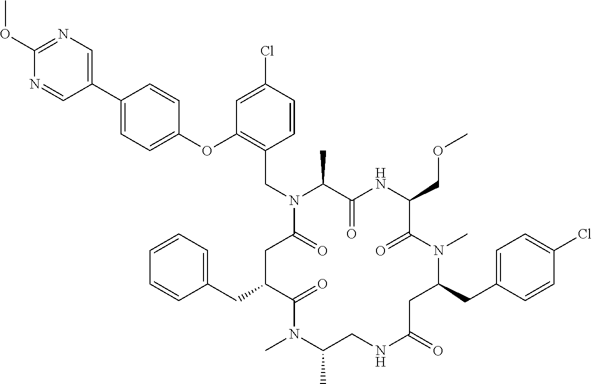 [SMILES: COC[C@@H]1NC(=O)[C@H](C)N(Cc2ccc(Cl)cc2Oc2ccc(-c3cnc(OC)nc3)cc2)C(=O)C[C@@H](Cc2ccccc2)C(=O)N(C)[C@@H](C)CNC(=O)C[C@H](Cc2ccc(Cl)cc2)N(C)C1=O] | Compound 53 (Example 2.2) | Analytical method 6 t_{R} = 3.08 min [M + H]⁺ = 966.4 |
| 86 | 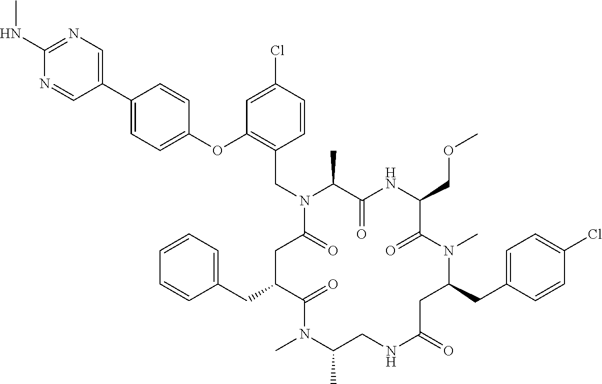 [SMILES: CNc1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)cn1] | Compound 53 (Example 2.2) | Analytical method 6 t_{R} = 2.91 min [M + H]⁺ = 965.4 |
| 88 | 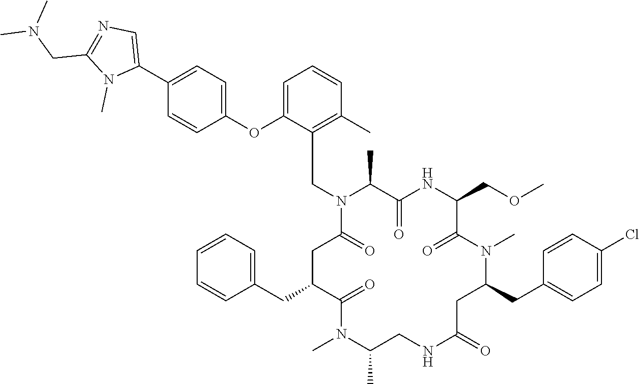 [SMILES: COC[C@@H]1NC(=O)[C@H](C)N(Cc2c(C)cccc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 53 (Example 2.2) | Analytical method 2 t_{R} = 2.77 min [M + H]⁺ = 975.6 |
| 118A | 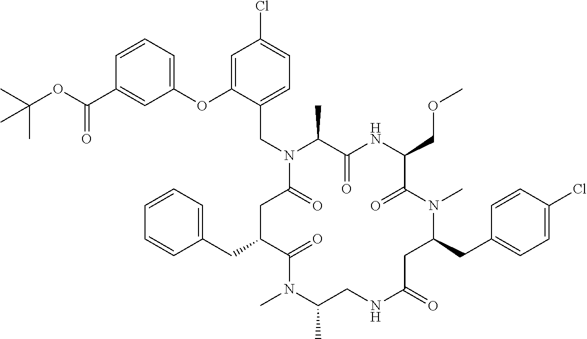 [SMILES: COC[C@@H]1NC(=O)[C@H](C)N(Cc2ccc(Cl)cc2Oc2cccc(C(=O)OC(C)(C)C)c2)C(=O)C[C@@H](Cc2ccccc2)C(=O)N(C)[C@@H](C)CNC(=O)C[C@H](Cc2ccc(Cl)cc2)N(C)C1=O] | Compound 53 (Example 2.2) | Analytical method 5 t_{R} = 1.58 min [M + H]⁺ = 958.8 |
| 125A | 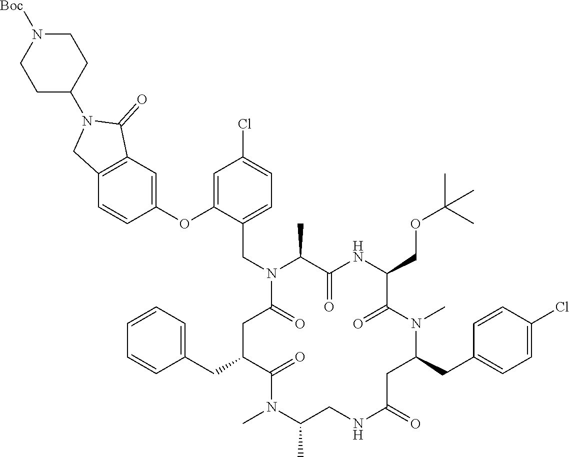 [SMILES: C[C@H]1CNC(=O)C[C@H](Cc2ccc(Cl)cc2)N(C)C(=O)[C@H](COC(C)(C)C)NC(=O)[C@H](C)N(Cc2ccc(Cl)cc2Oc2ccc3c(c2)C(=O)N(C2CCN(C(=O)OC(C)(C)C)CC2)C3)C(=O)C[C@@H](Cc2ccccc2)C(=O)N1C] | Compound 53 (Example 2.2) | Analytical method 7 t_{R} = 1.54 min [M + H]⁺ = 1138.5 |
| 142A | 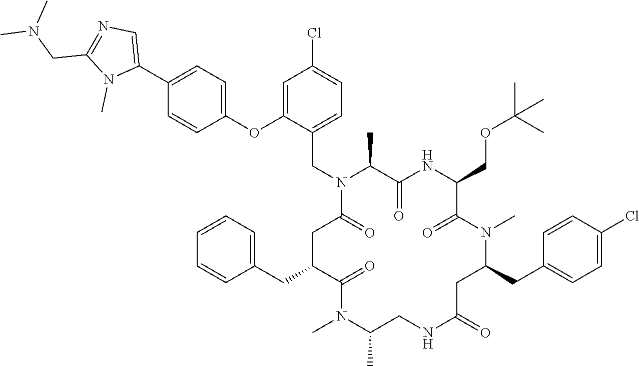 [SMILES: C[C@H]1CNC(=O)C[C@H](Cc2ccc(Cl)cc2)N(C)C(=O)[C@H](COC(C)(C)C)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Compound 53 (Example 2.2) | Analytical method 5 t_{R} = 1.43 min [M + H]⁺ = 1037.5 |
| 143A | 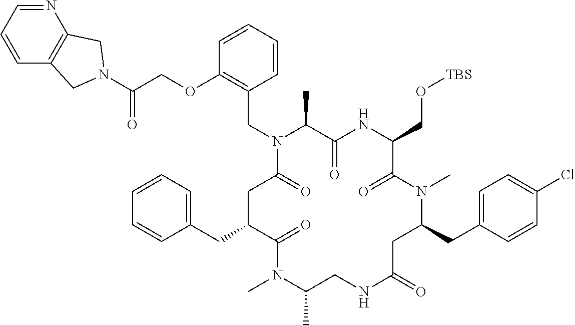 | Compound 53 (Example 2.2) | Analytical method 1 t_{R} = 1.74 min [M + H]⁺ = 1008.5 |
| 144 | 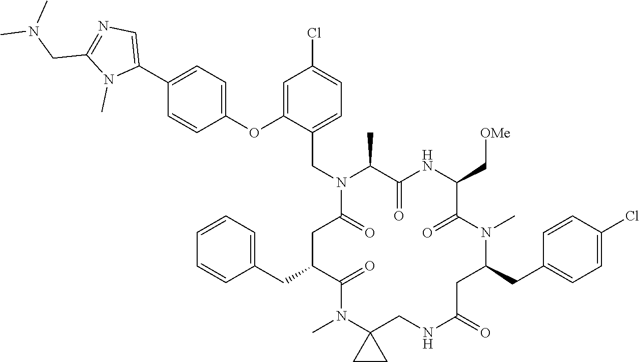 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)C2(CC2)CNC(=O)C[C@H](Cc2ccc(Cl)cc2)N(C)C1=O] | Compound 53 (Example 2.2) | Analytical method 4 t_{R} = 2.17 min [M + H]⁺ = 1007.3 |
| 146A | 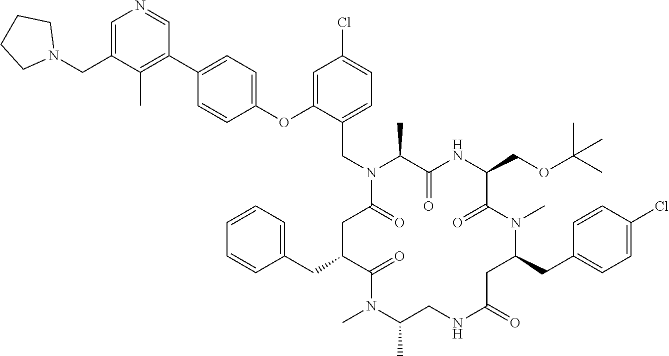 [SMILES: Cc1c(CN2CCCC2)cncc1-c1ccc(Oc2cc(Cl)ccc2CN2C(=O)C[C@@H](Cc3ccccc3)C(=O)N(C)[C@@H](C)CNC(=O)C[C@H](Cc3ccc(Cl)cc3)N(C)C(=O)[C@H](COC(C)(C)C)NC(=O)[C@@H]2C)cc1] | Compound 53 (Example 2.2) | Analytical method 7 t_{R} = 1.26 min [M + H]⁺ = 1074.4 |
| 148A | 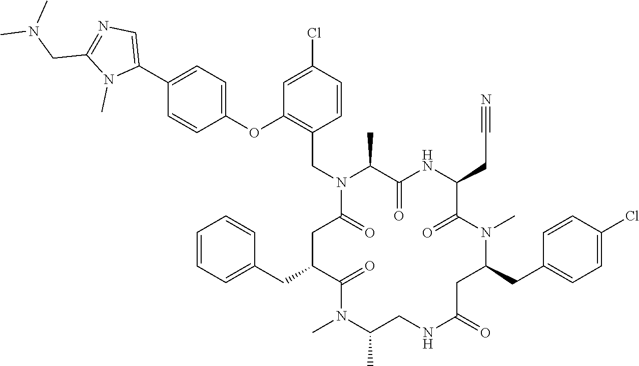 [SMILES: C[C@H]1CNC(=O)C[C@H](Cc2ccc(Cl)cc2)N(C)C(=O)[C@H](CC#N)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Compound 53 (Example 2.2) | Analytical method 9 t_{R} = 1.06 min [M + H]⁺ = 990.4 |
| 151A | 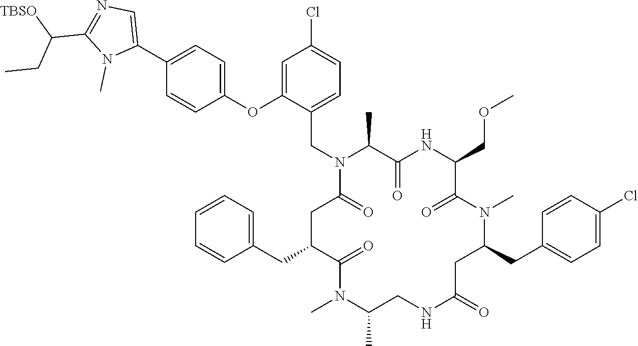 [SMILES: CCC(O[Si](C)(C)C(C)(C)C)c1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)n1C] | Compound 53 (Example 2.2) | Analytical method 5 t_{R} = 1.75 min [M + H]⁺ = 1110.4 |
| 154 | 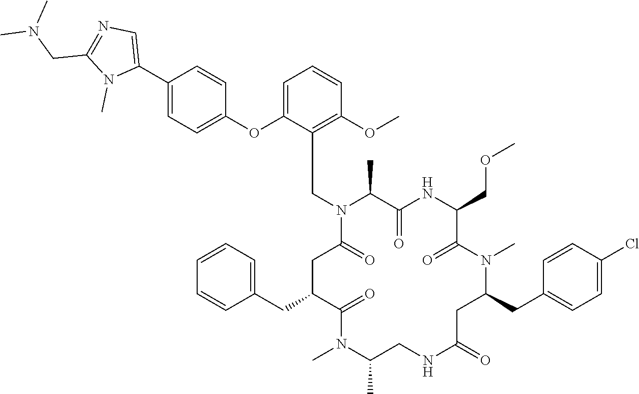 [SMILES: COC[C@@H]1NC(=O)[C@H](C)N(Cc2c(OC)cccc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 53 (Example 2.2) | Analytical method 2 t_{R} = 2.76 min [M + H]⁺ = 991.0 |
| 161 | 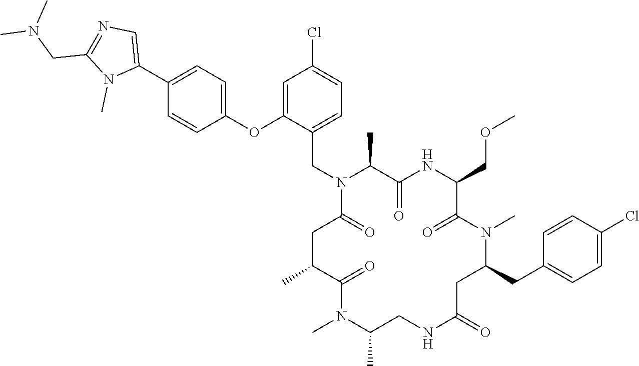 [SMILES: COC[C@@H]1NC(=O)[C@H](C)N(Cc2ccc(Cl)cc2Oc2ccc(-c3cnc(CN(C)C)n3C)cc2)C(=O)C[C@@H](C)C(=O)N(C)[C@@H](C)CNC(=O)C[C@H](Cc2ccc(Cl)cc2)N(C)C1=O] | Compound 53 (Example 2.2) | Analytical method 1 t_{R} = 1.09 min [M + H]⁺ = 920.0 |
| 167A | 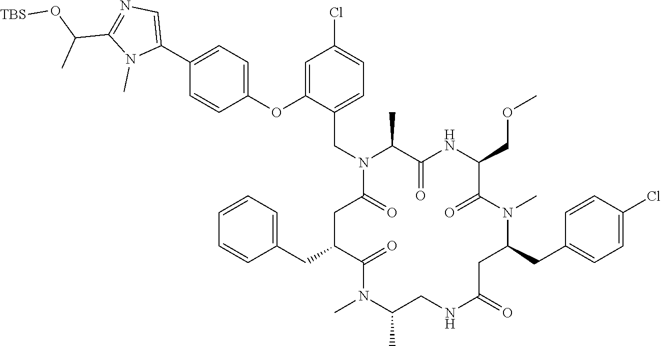 | Compound 53 (Example 2.2) | Analytical method 5 t_{R} = 1.66 min [M + H]⁺ = 1096.3 |
| 173 | 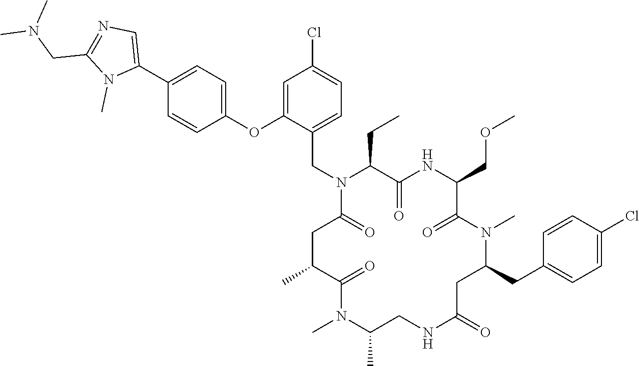 [SMILES: CC[C@H]1C(=O)N[C@@H](COC)C(=O)N(C)[C@@H](Cc2ccc(Cl)cc2)CC(=O)NC[C@H](C)N(C)C(=O)[C@H](C)CC(=O)N1Cc1ccc(Cl)cc1Oc1ccc(-c2cnc(CN(C)C)n2C)cc1] | Compound 53 (Example 2.2) | Analytical method 2 t_{R} = 2.64 min [M + H]⁺ = 932.9 |
| 181 | 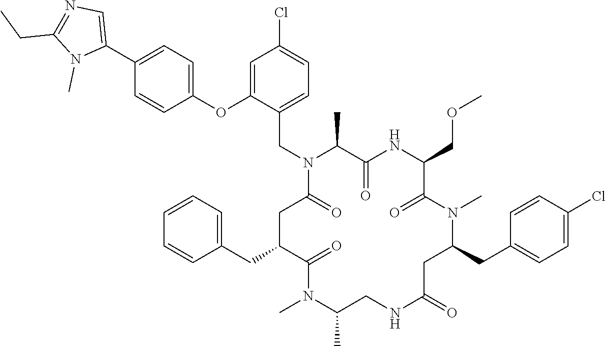 [SMILES: CCc1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)n1C] | Compound 53 (Example 2.2) | Analytical method 2 t_{R} = 2.89 min [M + H]⁺ = 966.3 |
| 183 | 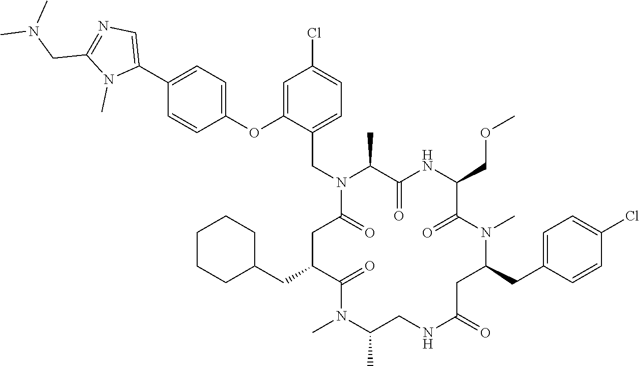 [SMILES: COC[C@@H]1NC(=O)[C@H](C)N(Cc2ccc(Cl)cc2Oc2ccc(-c3cnc(CN(C)C)n3C)cc2)C(=O)C[C@@H](CC2CCCCC2)C(=O)N(C)[C@@H](C)CNC(=O)C[C@H](Cc2ccc(Cl)cc2)N(C)C1=O] | Compound 53 (Example 2.2) | Analytical method 2 t_{R} = 3.09 min [M + H]⁺ = 1003.0 |
| 193 | 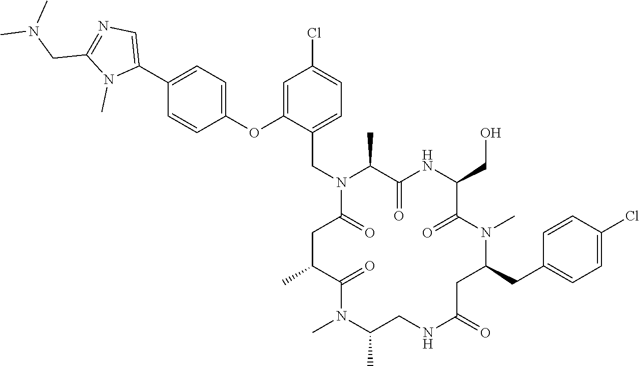 [SMILES: C[C@@H]1CC(=O)N(Cc2ccc(Cl)cc2Oc2ccc(-c3cnc(CN(C)C)n3C)cc2)[C@@H](C)C(=O)N[C@@H](CO)C(=O)N(C)[C@@H](Cc2ccc(Cl)cc2)CC(=O)NC[C@H](C)N(C)C1=O] | Compound 53 (Example 2.2)/The linear peptide was cleaved from the resin using TFA/TIS/H₂O (95:2.5:2.5). The obtained TFA salt was converted to the HCl salt by treatment with 4M HCl in dioxane | Analytical method 7 t_{R} = 0.89 min [M + H]⁺ = 905.5 |
| 200A | 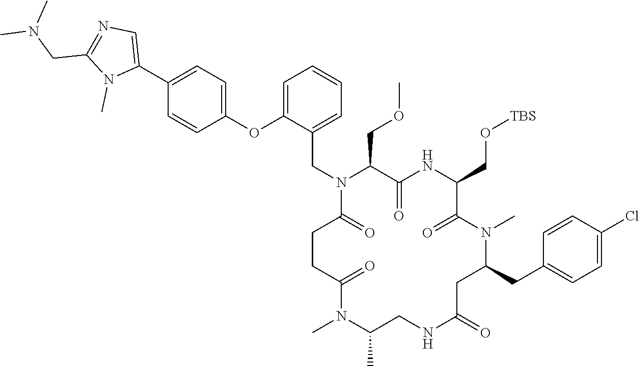 | Compound 53 (Example 2.2) | Analytical method 5 t_{R} = 1.45 min [M + H]⁺ = 1001.3 |
| 202 | 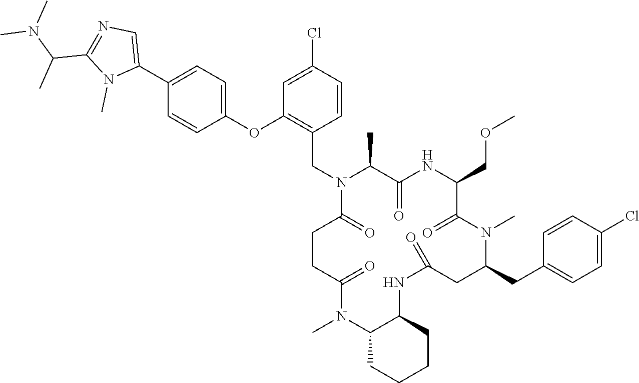 | Compound 53 (Example 2.2) | Analytical method 4 t_{R} = 1.96 min [M + H]⁺ = 945.5 |
| 209 | 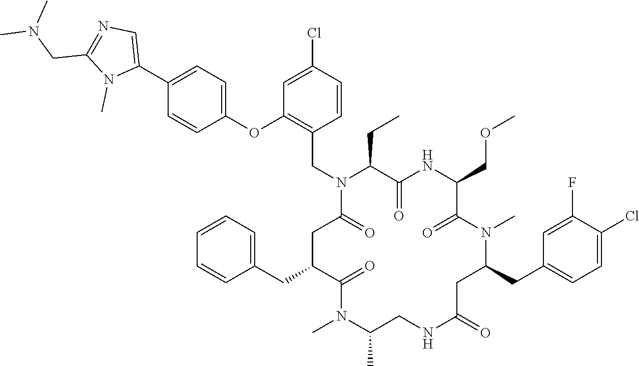 [SMILES: CC[C@H]1C(=O)N[C@@H](COC)C(=O)N(C)[C@@H](Cc2ccc(Cl)c(F)c2)CC(=O)NC[C@H](C)N(C)C(=O)[C@H](Cc2ccccc2)CC(=O)N1Cc1ccc(Cl)cc1Oc1ccc(-c2cnc(CN(C)C)n2C)cc1] | Compound 53 (Example 2.2) | Analytical method 7 t_{R} = 1.00 min [M + H]⁺ = 1027.3 |
| 210 | 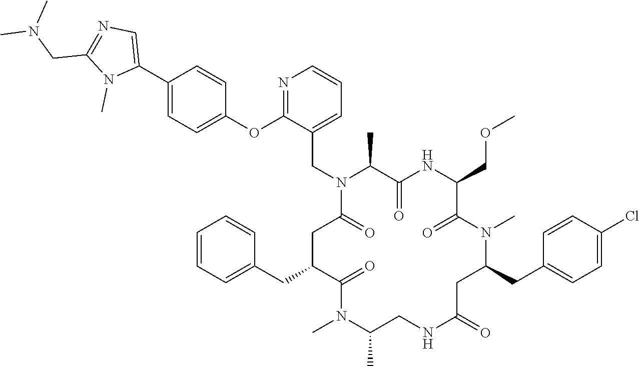 [SMILES: COC[C@@H]1NC(=O)[C@H](C)N(Cc2cccnc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 53 (Example 2.2) | Analytical method 7 t_{R} = 0.97 min [M + H]⁺ = 962.4 |
| 214 | 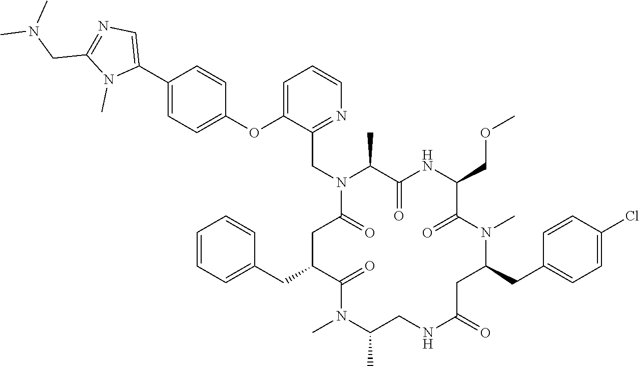 [SMILES: COC[C@@H]1NC(=O)[C@H](C)N(Cc2ncccc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 53 (Example 2.2) | Analytical method 7 t_{R} = 1.00 min [M + H]⁺ = 962.3 |
| 233A | 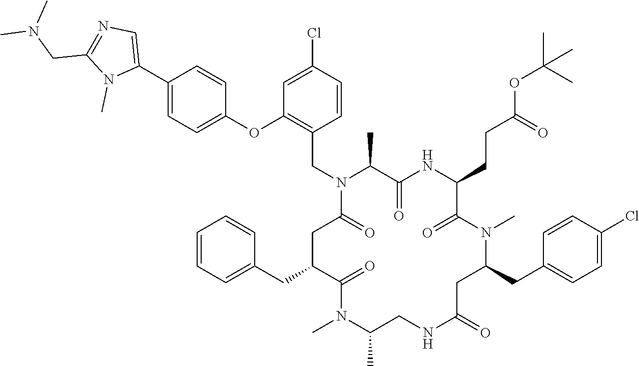 [SMILES: C[C@H]1CNC(=O)C[C@H](Cc2ccc(Cl)cc2)N(C)C(=O)[C@H](CCC(=O)OC(C)(C)C)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Compound 53 (Example 2.2) | Analytical method 5 t_{R} = 1.31 min [M − H]⁻ = 1077.3 |
| 242A | 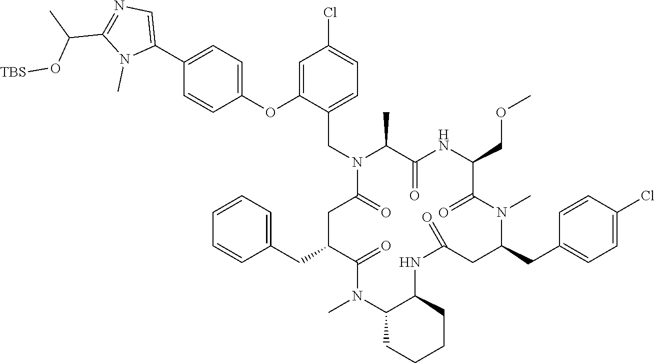 | Compound 53 (Example 2.2) | Analytical method 5 t_{R} = 1.72 min [M + H]⁺ = 1138.2 |
| 248 | 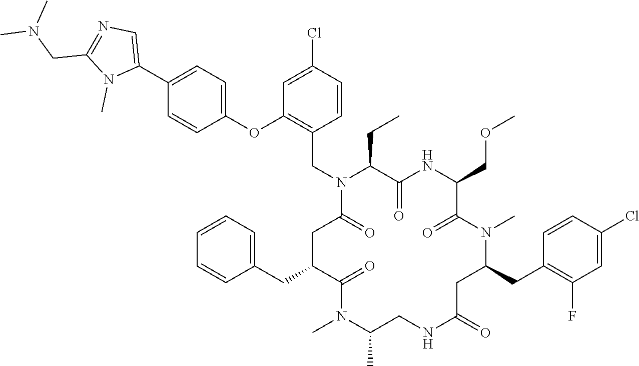 [SMILES: CC[C@H]1C(=O)N[C@@H](COC)C(=O)N(C)[C@@H](Cc2ccc(Cl)cc2F)CC(=O)NC[C@H](C)N(C)C(=O)[C@H](Cc2ccccc2)CC(=O)N1Cc1ccc(Cl)cc1Oc1ccc(-c2cnc(CN(C)C)n2C)cc1] | Compound 53 (Example 2.2) | Analytical method 7 t_{R} = 1.06 min [M + H]⁺ = 1026.8 |
| 282 | 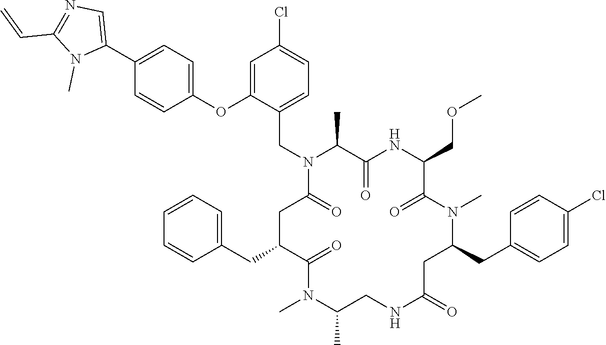 [SMILES: C=Cc1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)n1C] | Compound 53 (Example 2.2)/ NaBH(OAc)₃ was used for reductive alkylation (Step 1). | Analytical method 2 t_{R} = 2.90 min [M + H]⁺ = 964.3 |

### Example 2.3: Method C— Synthesis of (4S,7S,10S,14R,16aS,20aS)-10-(2-aminoethyl)-14-benzyl-11-(4-chloro-2-(4-(2-((dimethylamino)methyl)-1-methyl-1H-imidazol-5-yl)phenoxy)-benzyl)-4-(4-chlorobenzyl)-7-(methoxymethyl)-5,16-dimethylhexadecahydro-benzo[l][1,4,7,11,14]pentaazacyclo-octadecine-2,6,9,12,15(3H)-pentaone (Compound 109)

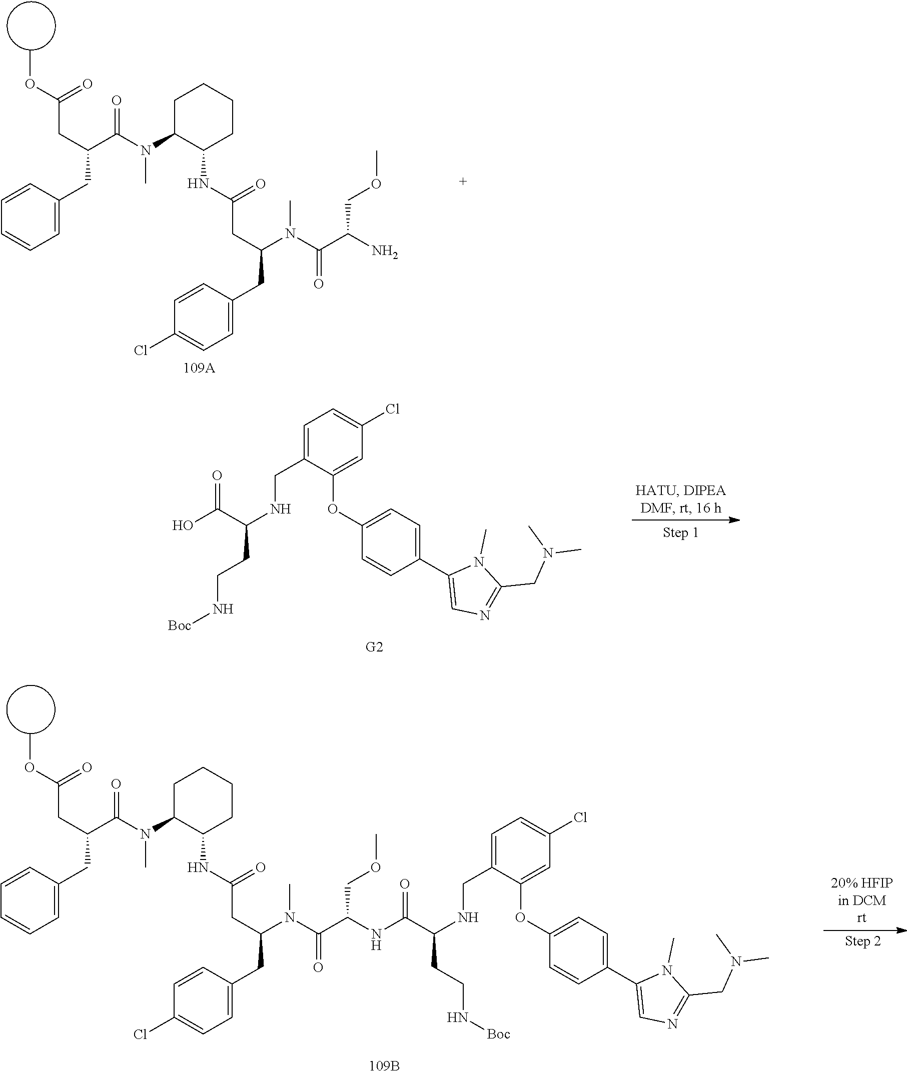 [SMILES: COC[C@H](N)C(=O)N(C)[C@H](CC(=O)N[C@H]1CCCC[C@@H]1N(C)C(=O)[C@@H](CC(=O)OC)Cc1ccccc1)Cc1ccc(Cl)cc1 ; CN(C)Cc1ncc(-c2ccc(Oc3cc(Cl)ccc3CN[C@@H](CCNC(=O)OC(C)(C)C)C(=O)O)cc2)n1C ; COC[C@H](NC(=O)[C@H](CCNC(=O)OC(C)(C)C)NCc1ccc(Cl)cc1Oc1ccc(-c2cnc(CN(C)C)n2C)cc1)C(=O)N(C)[C@H](CC(=O)N[C@H]1CCCC[C@@H]1N(C)C(=O)[C@@H](CC(=O)OC)Cc1ccccc1)Cc1ccc(Cl)cc1]

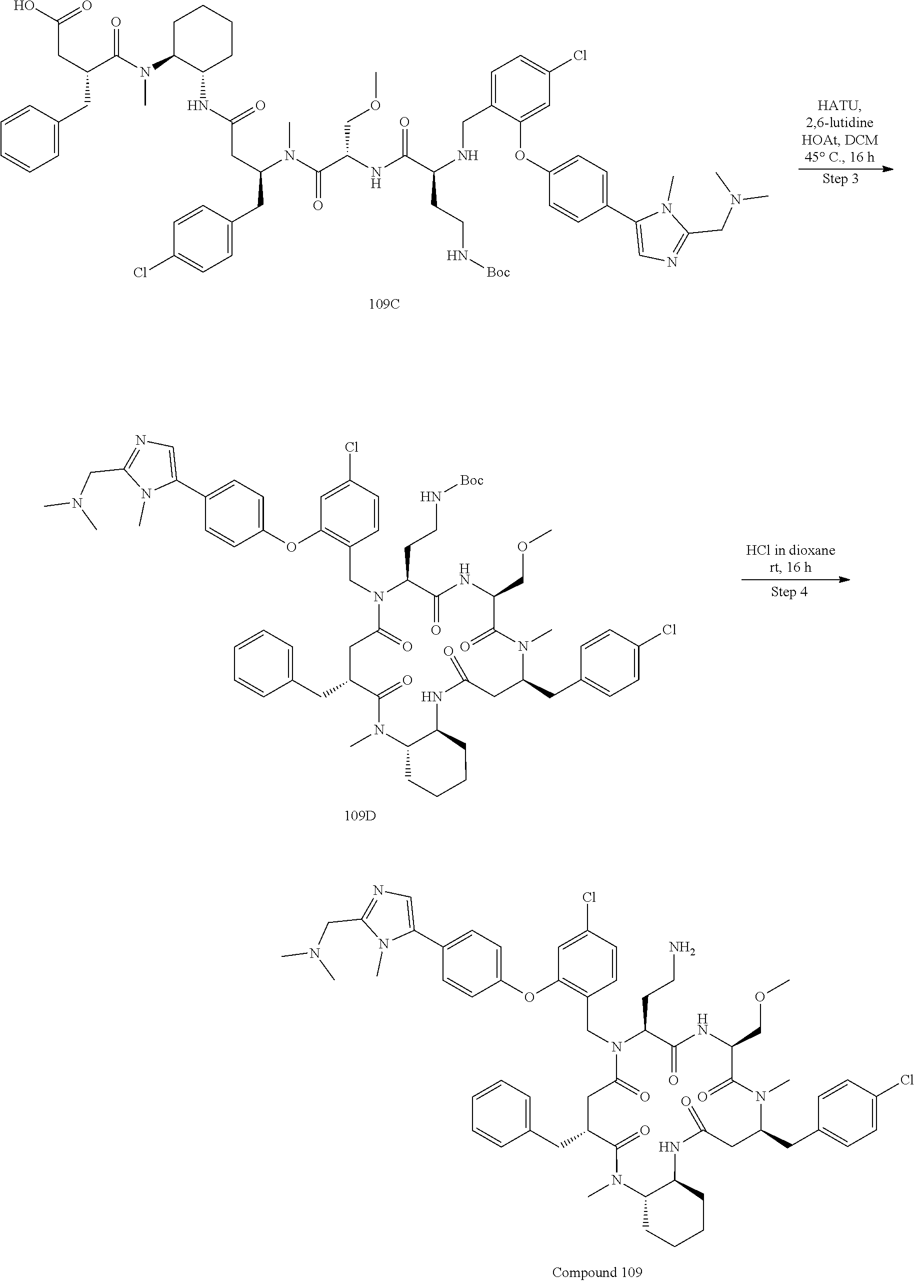

### Step 1. PS-2-chlorotrityl (R)-3-benzyl-4-(((1S,2S)-2-((8S,11S,14S)-8-((4-chloro-2-(4-(2-((dimethylamino)-methyl)-1-methyl-1H-imidazol-5-yl)phenoxy)benzyl)amino)-14-(4-chlorobenzyl)-1-(methoxymethyl)-2,2,13-trimethyl-4,9,2-trioxo-3-oxa-5,10,13-triazahexadecan-16-amido)cyclohexyl)(methyl)amino)-4-oxobutanoate (109B)

To G2 (1.000 g, 1.680 mmol) in DMF (20 mL) was added DIPEA (0.587 mL, 3.36 mmol) followed by HATU (0.639 g, 1.680 mmol) and the resulting mixture was stirred until completely homogenous. This solution was then added to 109A (prepared according to the procedure used for 150B in Example 1, Step 2) (2.8 g, 0.840 mmol) in 10 mL of DMF in a shaker flask. The reaction mixture was allowed to shake o/n at rt. The resin was filtered, washed with DMF (3×) and DCM (3×), and dried under vacuum to give 109B (2.9 g, crude) which was taken onto the next step without purification.

### Step 2. (R)-3-Benzyl-4-(((1S,2S)-2-((8S,11S,14S)-8-((4-chloro-2-(4-(2-((dimethylamino)methyl)-1-methyl-1H-imidazol-5-yl)phenoxy)benzyl)amino)-14-(4-chlorobenzyl)-11-(methoxymethyl)-2,2,13-trimethyl-4,9,12-trioxo-3-oxa-5,10,13-triazahexadecan-16-amido)cyclohexyl)(methyl)amino)-4-oxobutanoic acid (109C)

109B (2.9 g, 0.87 mmol) was cleaved from the resin by shaking with 75 mL of 20% HFIP/DCM for 20 min at rt. The resin was filtered and the filtrate was collected. Both steps were repeated three more times to ensure all of the product was cleaved from the resin. The combined filtrates were concentrated to provide a crude oil (1.8 g) which was purified via reversed-phase chromatography (eluting with MeCN/H₂O with 0.1% NH₄OH) to afford 109C as a white solid (917 mg, 80%). Analytical method 2; t_{R}=2.04 min; [M+H]⁺=1182.7.

### Step 3. tert-Butyl (2-((4S,7S,10S,14R,16aS,20aS)-14-benzyl-11-(4-chloro-2-(4-(2-((dimethylamino)-methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)ethyl)carbamate trifluoroacetate (109D)

To 109C (800 mg, 0.676 mmol) dissolved in DCM (800 mL) was added HATU (1028 mg, 2.70 mmol), 2,6-lutidine (2.362 mL, 20.28 mmol) and HOAt (92 mg, 0.676 mmol) and the resulting mixture was stirred for 18 h at 45° C. The reaction mixture was then concentrated to dryness via rotary evaporation with water bath at rt. The obtained crude oil was purified via reversed-phase chromatography (eluting with ACN/H₂O with 0.1% TFA) to afford 109D as a white solid (484 mg, 59.6%). Analytical method 2; t_{R}=3.18 min; [M+H]⁺=1166.8.

### Step 4. (4S,7S,10S,14R,16aS,20aS)-10-(2-Aminoethyl)-14-benzyl-11-(4-chloro-2-(4-(2-((dimethyl amino)methyl)-1-methyl-1H-imidazol-5-yl)phenoxy)benzyl)-4-(4-chlorobenzyl)-7-(methoxymethyl)-5,16-dimethylhexadecahydrobenzo[l][1,4,7,11,14]pentaazacyclooctadecine-2,6,9,12,15(3H)-pentaone (Compound 109)

To a solution of 1090 (484 mg, 0.415 mmol) in anhydrous dioxane (20 ml) at 0° C. was added HCl (4 M in dioxane) (7.27 mL, 29.1 mmol) dropwise. The reaction mixture turned cloudy. After 15 m the ice bath was removed and the mixture was then stirred at rt overnight. The reaction mixture was then concentrated via rotary evaporation with water bath at rt and the resulting residue was dried under vacuum. The crude material was dissolved in EtOAc and washed with sat. aq. sodium bicarbonate (2×300 mL). The aq. layer was then extracted with fresh EtOAc (×5). The combined organics were dried over sodium sulfate, filtered, and concentrated in vacuo to yield Compound 109 as a white solid (370 mg, 95%). Analytical method 7; t_{R}=0.87 min; [M+H]⁺=1063.7.

The Compounds and intermediates shown in Table 3 below were prepared according to the procedure used for Compound 109 or Intermediate 109D (Example 2.3) from the respective intermediates shown in in Table 19, Table 21, Table 22, and Table 31.

TABLE 3

| Cmd | | Synthesis | |
|---|---|---|---|
| No. | Structure | procedure | LCMS |
| 4 | 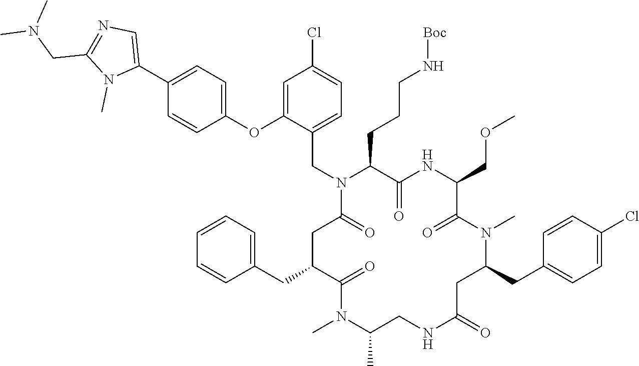 [SMILES: COC[C@@H]1NC(=O)[C@H](CCCNC(=O)OC(C)(C)C)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Intermediate 109D (Example 2.3) | Analytical method 3 t_{R} = 1.11 min [M + H]⁺ = 1138.5 |
| 31 | 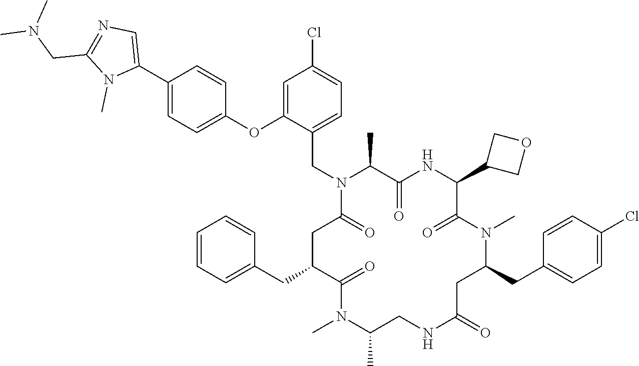 [SMILES: C[C@H]1CNC(=O)C[C@H](Cc2ccc(Cl)cc2)N(C)C(=O)[C@H](C2COC2)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Intermediate 109D (Example 2.3) | Analytical method 7 t_{R} = 0.92 min [M − H]⁻ = 1005.4 |
| 79 | 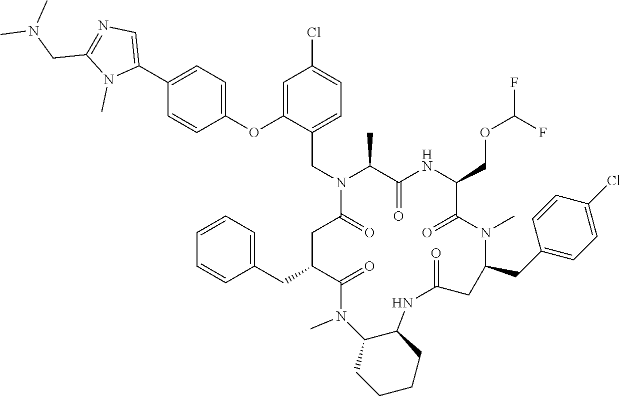 [SMILES: C[C@H]1C(=O)N[C@@H](COC(F)F)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1] | Intermediate 109D (Example 2.3) | Analytical method 2 t_{R} = 3.10 min [M + H]⁺ = 1071.5 |
| 94 | 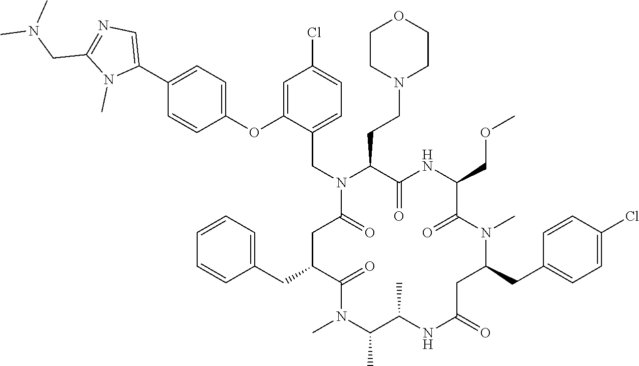 [SMILES: COC[C@@H]1NC(=O)[C@H](CCN2CCOCC2)N(Cc2ccc(Cl)cc2Oc2ccc(-c3cnc(CN(C)C)n3C)cc2)C(=O)C[C@@H](Cc2ccccc2)C(=O)N(C)[C@@H](C)[C@H](C)NC(=O)C[C@H](Cc2ccc(Cl)cc2)N(C)C1=O] | Intermediate 109D (Example 2.3) | Analytical method 3 t_{R} = 0.87 min [M + H]⁺ = 1108.5 |
| 113A | 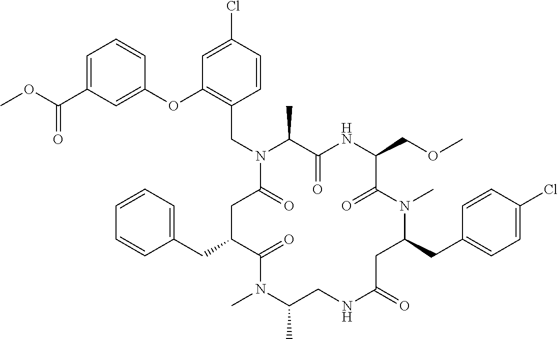 [SMILES: COC[C@@H]1NC(=O)[C@H](C)N(Cc2ccc(Cl)cc2Oc2cccc(C(=O)OC)c2)C(=O)C[C@@H](Cc2ccccc2)C(=O)N(C)[C@@H](C)CNC(=O)C[C@H](Cc2ccc(Cl)cc2)N(C)C1=O] | Intermediate 109D (Example 2.3)- from Fmoc- protected G11 and G9, respectively. Fmoc was removed by treatment with 4- methypiperidine prior to cleavage from the resin. | Analytical method 15 t_{R} = 2.89 min [M + H]⁺ = 916.3 |
| 120A | 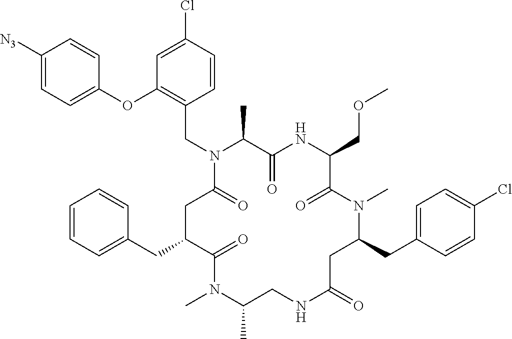 [SMILES: COC[C@@H]1NC(=O)[C@H](C)N(Cc2ccc(Cl)cc2Oc2ccc(N=[N+]=[N-])cc2)C(=O)C[C@@H](Cc2ccccc2)C(=O)N(C)[C@@H](C)CNC(=O)C[C@H](Cc2ccc(Cl)cc2)N(C)C1=O] | Intermediate 109D (Example 2.3)- from Fmoc- protected G11 and G9, respectively. Fmoc was removed by treatment with 4- methypiperidine prior to cleavage from the resin. | Analytical method 4 t_{R} = 3.29 min [M + H]⁺ = 899.7 |
| 170 | 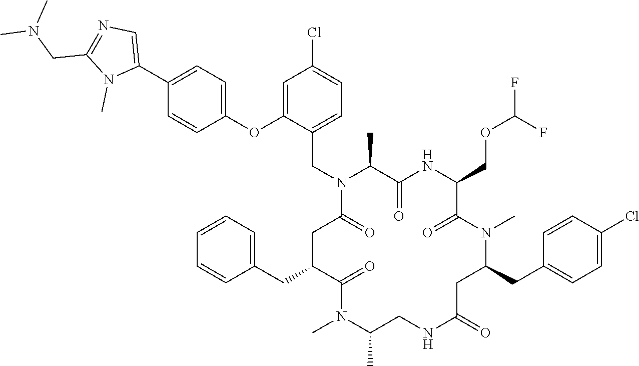 [SMILES: C[C@H]1CNC(=O)C[C@H](Cc2ccc(Cl)cc2)N(C)C(=O)[C@H](COC(F)F)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Intermediate 109D (Example 2.3) | Analytical method 2 t_{R} = 2.92 min [M + H]⁺ = 1031.4 |
| 182 | 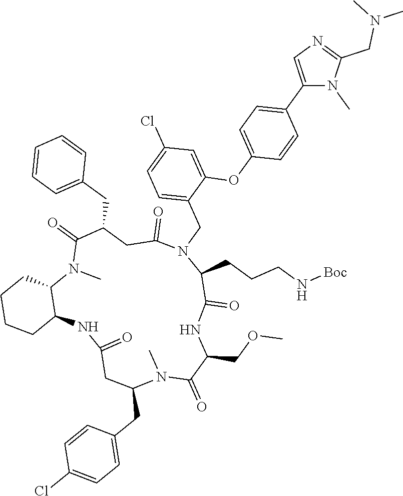 [SMILES: COC[C@@H]1NC(=O)[C@H](CCCNC(=O)OC(C)(C)C)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Intermediate 109D (Example 2.3) | Analytical method 5 t_{R} = 1.28 min [M + H]⁺ = 1177.6 |
| 186 | 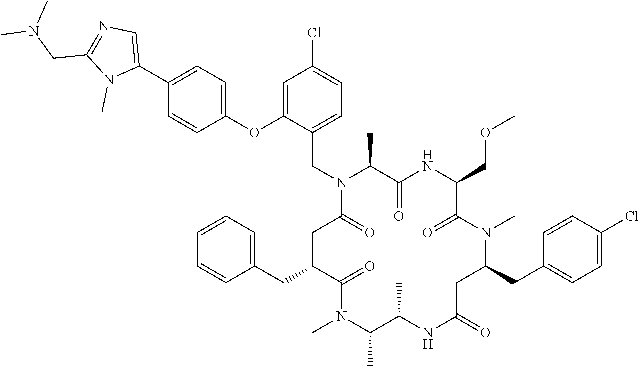 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H](C)[C@H](C)NC(=O)C[C@H](Cc2ccc(Cl)cc2)N(C)C1=O] | Intermediate 109D (Example 2.3) | Analytical method 3 t_{R} = 1.07 min [M + H]⁺ = 1009.6 |
| 191 | 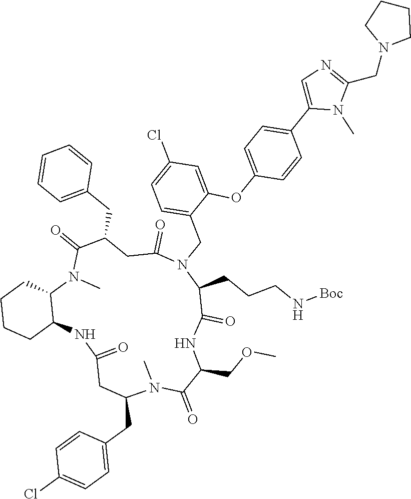 [SMILES: COC[C@@H]1NC(=O)[C@H](CCCNC(=O)OC(C)(C)C)N(Cc2ccc(Cl)cc2Oc2ccc(-c3cnc(CN4CCCC4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Intermediate 109D (Example 2.3) | Analytical method 7 t_{R} = 1.07 min [M + H]⁺ = 1204.3 |
| 192 | 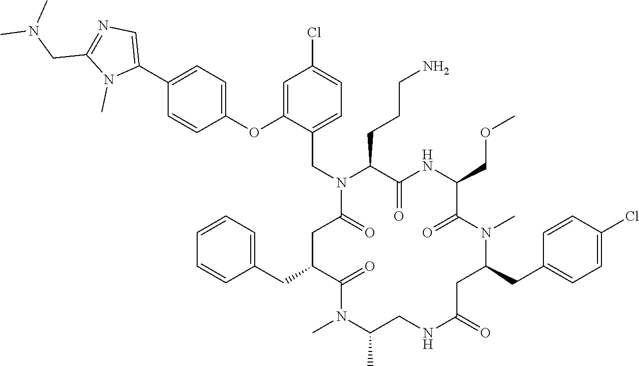 [SMILES: COC[C@@H]1NC(=O)[C@H](CCCN)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 109 (Example 2.3) | Analytical method 5 t_{R} = 1.18 min [M + H]⁺ = 1038.4 |
| 198 | 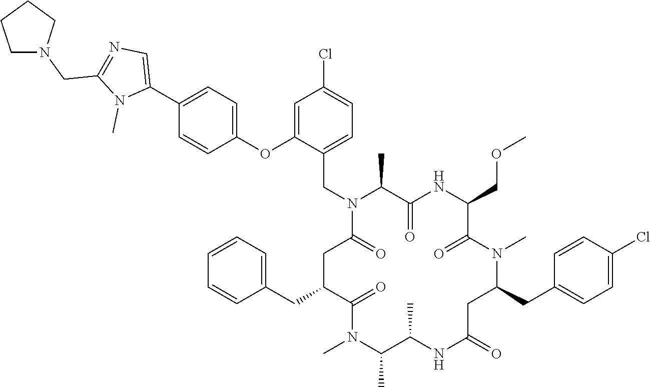 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](C)[C@H](C)NC(=O)C[C@H](Cc2ccc(Cl)cc2)N(C)C1=O] | Intermediate 109D (Example 2.3) | Analytical method 3 t_{R} = 1.07 min [M + H]⁺ = 1035.5 |
| 204 | 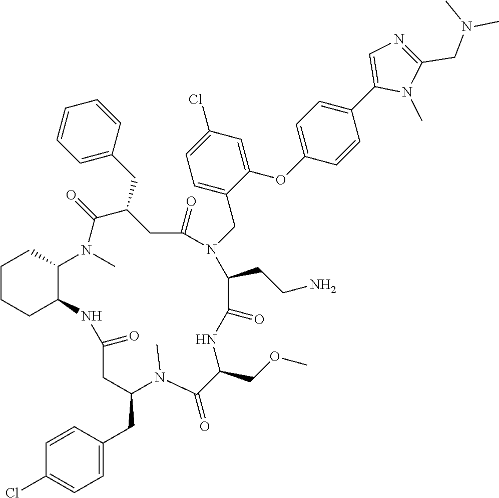 [SMILES: COC[C@@H]1NC(=O)[C@H](CCN)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 109 (Example 2.3) | Analytical method 5 t_{R} = 1.23 min [M + H]⁺ = 1064.7 |
| 252A | 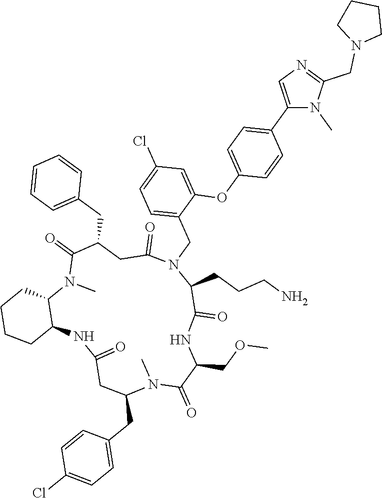 [SMILES: COC[C@@H]1NC(=O)[C@H](CCCN)N(Cc2ccc(Cl)cc2Oc2ccc(-c3cnc(CN4CCCC4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 109 (Example 2.3) | Analytical method 5 t_{R} = 1.35 min [M − H]⁻ = 1102.2 |
| 269 | 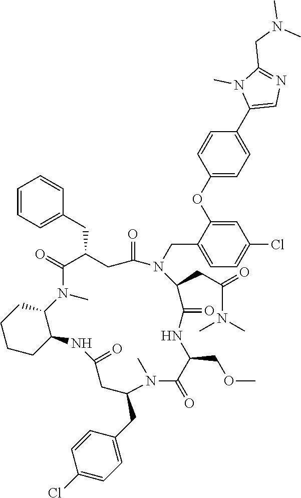 [SMILES: COC[C@@H]1NC(=O)[C@H](CC(=O)N(C)C)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Intermediate 109D (Example 2.3) | Analytical method 3 t_{R} = 1.11 min [M + H]⁺ = 1106.5 |
| 277 | 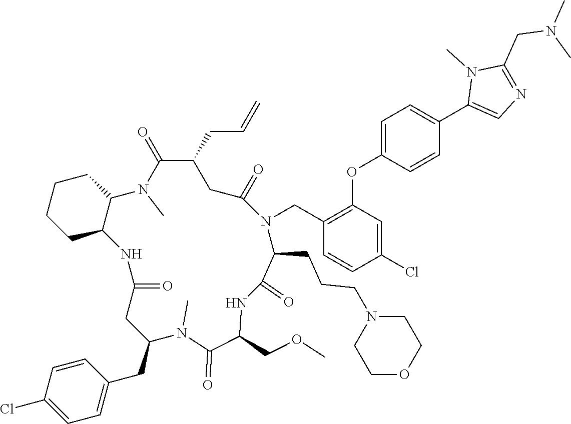 | Intermediate 109D (Example 2.3) | Analytical method 7 t_{R} = 1.03 min [M + H]⁺ = 989.5 |
| 283 | 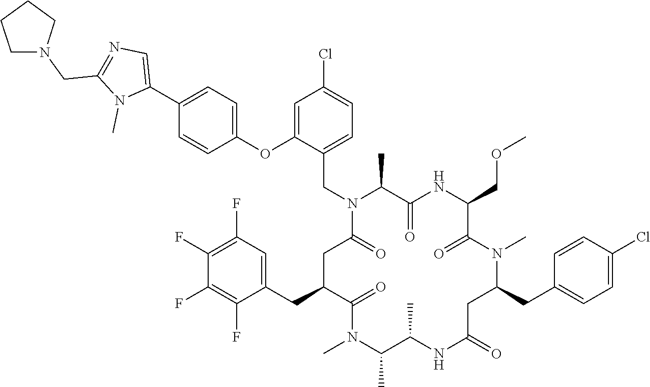 | Intermediate 109D (Example 2.3) | Analytical method 3 t_{R} = 0.87 min [M + H]⁺ = 1122.5 |
| 285A | 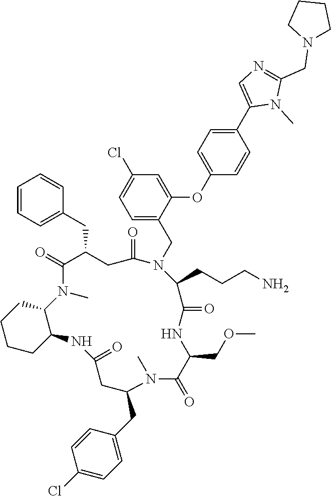 [SMILES: COC[C@@H]1NC(=O)[C@H](CCCN)N(Cc2ccc(Cl)cc2Oc2ccc(-c3cnc(CN4CCCC4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 109 (Example 2.3) | Analytical method 5 t_{R} = 1.31 min [M + H]⁺ = 1104.4 |
| 304 | 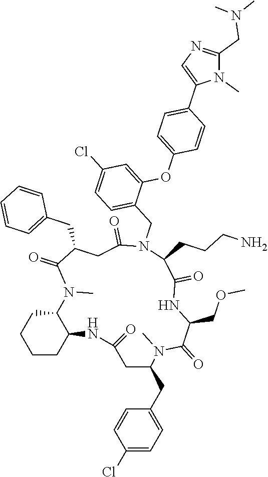 [SMILES: COC[C@@H]1NC(=O)[C@H](CCCN)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 109 (Example 2.3) | Analytical method 5 t_{R} = 1.21 min [M + H]⁺ = 1078.7 |

### Example 2.4: Method D—Synthesis of (5S,8S,11S,15R)-15-benzyl-12-(4-chloro-2-(4-(2-((dimethylamino)methyl)-1-methyl-1H-imidazol-5-yl)phenoxy)benzyl)-5-(4-chlorobenzyl)-8-(methoxymethyl)-6,11,17-trimethyl-2,6,9,12,17-pentaazabicyclo[16.1.0]nonadecane-3,7,10,13,16-pentaone trifluoroacetate (Compound 147)

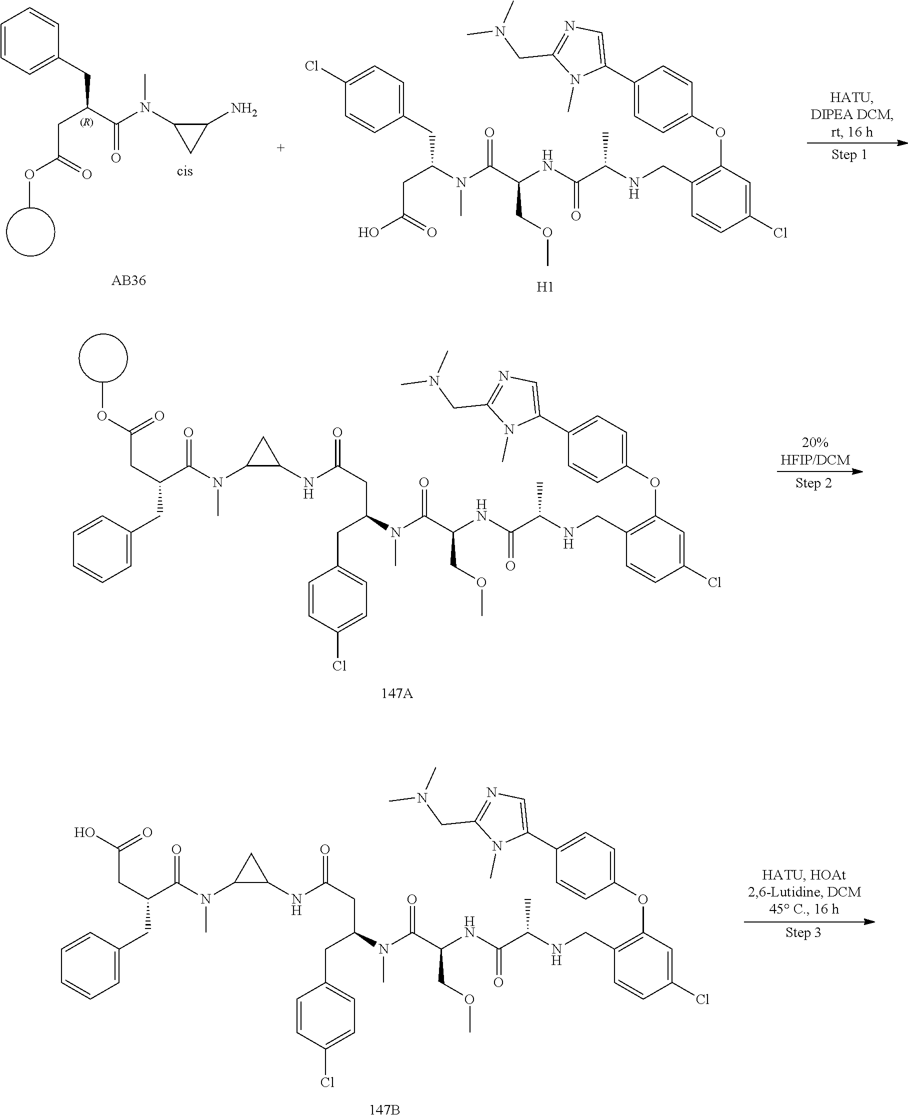

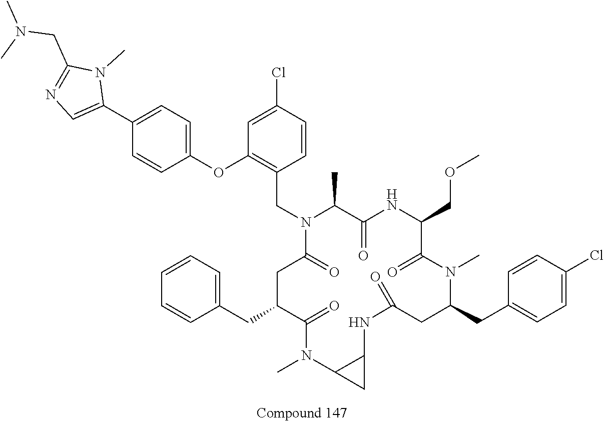 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)C2CC2NC(=O)C[C@H](Cc2ccc(Cl)cc2)N(C)C1=O]

### Step 1. PS-(2-Chlorotrityl) (3R)-3-benzyl-4-((2-((S)-3-((S)-2-((S)-2-((4-chloro-2-(4-(2-((dimethylamino)methyl)-1-methyl-1H-imidazol-5-yl)phenoxy)benzyl)amino)propanamido)-3-methoxy-N-methylpropanamido)-4-(4-chlorophenyl)butanamido)cyclopropyl)(methyl)amino)-4-oxobutanoate (147A)

147A was prepared according to the procedure used in Example 2.3, Step 1 starting from AB36 (cis racemic) (462 mg, 0.30 mmol) and H1 (338 mg, 0.45 mmol). Intermediate 147A (462 mg, crude), which was taken onto without purification.

### Step 2. (R)-3-benzyl-4-(((1S,2R)-2-((S)-3-((S)-2-((S)-2-((4-chloro-2-(4-(2-((dimethylamino)methyl)-1-methyl-1H-imidazol-5-yl)phenoxy)benzyl)amino)propanamido)-3-methoxy-N-methylpropanamido)-4-(4-chlorophenyl)butanamido)cyclopropyl)(methyl)amino)-4-oxobutanoic acid (147B)

147B was prepared according to the procedure used in Example 2.3, Step 2 starting from 147A (462 mg, 0.30 mmol). Analytical method 7; t_{R}=0.76 min; [M+H]⁺=1010.9.

### Step 3. (5S,8S,11S,15R)-15-benzyl-12-(4-chloro-2-(4-(2-((dimethylamino)methyl)-1-methyl-1H-imidazol-5-yl)phenoxy)benzyl)-5-(4-chlorobenzyl)-8-(methoxymethyl)-6,11,17-trimethyl-2,6,9,12,17-pentaazabicyclo-[16.1.0]nonadecane-3,7,10,13,16-pentaone trifluoroacetate (Compound 147)

Compound 147 was prepared according to the procedure used in Example 2.3, Step 3 starting from 147B (78 mg, 0.077 mmol). The TFA salt of Compound 147 (16.7 mg, 19%) was obtained as a white solid (cis diastereomeric mixture). Analytical method 4; t_{R}=1.96 min; [M+H]⁺=993.4.

The Compounds in Table 4 below were prepared according to the procedure used for Compound 147 (Example 2.4).

TABLE 4

| Cmd | | Synthesis | |
|---|---|---|---|
| No. | Structure | Procedure | LCMS |
| 61 | 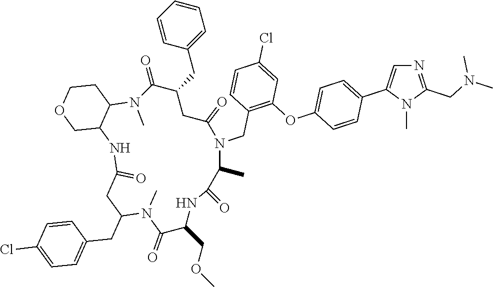 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)C2CCOCC2NC(=O)CC(Cc2ccc(Cl)cc2)N(C)C1=O] | Compound 147 (Example 2.4) | Analytical method 7 t_{R} = 0.96 min [M + H]⁺ = 1037.4 |
| 77 | 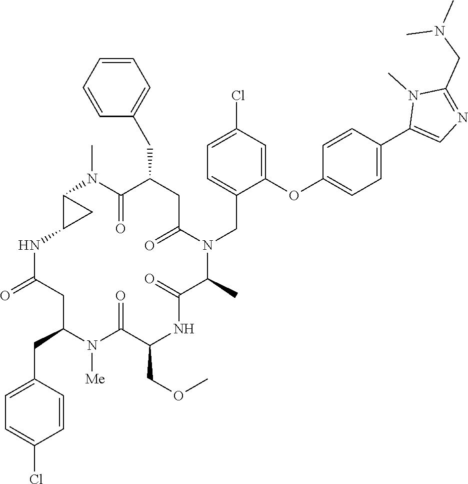 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H]2C[C@@H]2NC(=O)C[C@H](Cc2ccc(Cl)cc2)N(C)C1=O] | Compound 147 (Example 2.4)- obtained by chromatographic separation of Compound 147 using Chiral SFC conditions (35% MeOH:IPA (1:1) in CO₂ with 10 mM NH₄OH. Column OJ-H 21 × 250 mm 5 um | Analytical method 4 t_{R} = 1.94 min [M + H]⁺ = 993.6 |
| 89 | 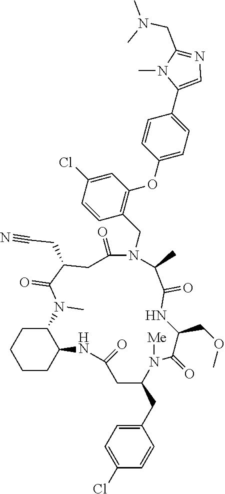 [SMILES: COC[C@@H]1NC(=O)[C@H](C)N(Cc2ccc(Cl)cc2Oc2ccc(-c3cnc(CN(C)C)n3C)cc2)C(=O)C[C@@H](CC#N)C(=O)N(C)[C@H]2CCCC[C@@H]2NC(=O)C[C@H](Cc2ccc(Cl)cc2)N(C)C1=O] | Compound 147 (Example 2.4) | Analytical method 4 t_{R} = 1.86 min [M + H]⁺ = 984.8 |
| 159 | 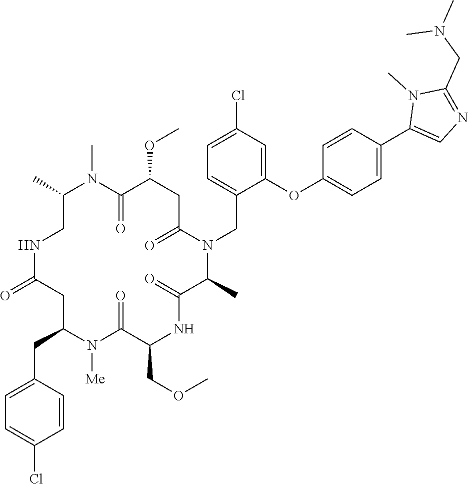 [SMILES: COC[C@@H]1NC(=O)[C@H](C)N(Cc2ccc(Cl)cc2Oc2ccc(-c3cnc(CN(C)C)n3C)cc2)C(=O)C[C@@H](OC)C(=O)N(C)[C@@H](C)CNC(=O)C[C@H](Cc2ccc(Cl)cc2)N(C)C1=O] | Compound 147 (Example 2.4) | Analytical method 4 t_{R} = 1.75 min [M + H]⁺ = 935.2 |
| 215 | 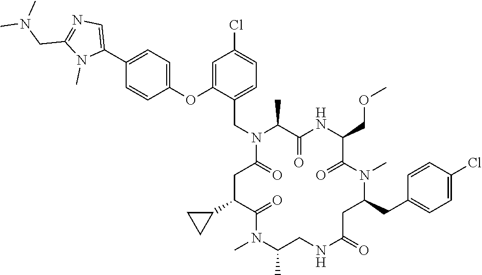 [SMILES: COC[C@@H]1NC(=O)[C@H](C)N(Cc2ccc(Cl)cc2Oc2ccc(-c3cnc(CN(C)C)n3C)cc2)C(=O)C[C@@H](C2CC2)C(=O)N(C)[C@@H](C)CNC(=O)C[C@H](Cc2ccc(Cl)cc2)N(C)C1=O] | Compound 147 (Example 2.4) | Analytical method 3 t_{R} = 0.99 min [M + H]⁺ = 945.4 |
| 247 | 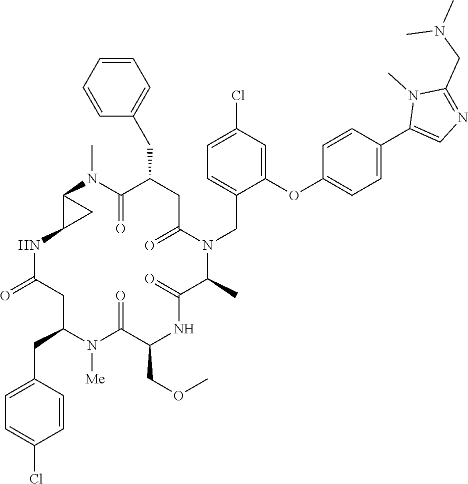 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@H]2C[C@H]2NC(=O)C[C@H](Cc2ccc(Cl)cc2)N(C)C1=O] | Compound 147 (Example 2.4)- obtained by chromatographic separation of Compound 147 using Chiral SFC conditions (35% MeOH:IPA (1:1) in CO₂ with 10 mM NH₄OH. Column OJ-H 21 × 250 mm 5 um) | Analytical method 4 t_{R} = 1.97 min [M + H]⁺ = 993.5 |
| 263 | 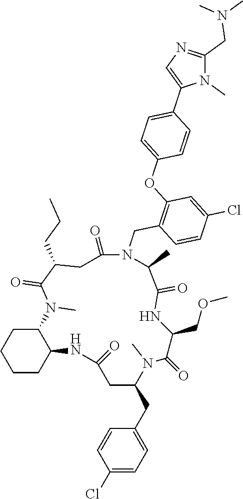 [SMILES: CCC[C@@H]1CC(=O)N(Cc2ccc(Cl)cc2Oc2ccc(-c3cnc(CN(C)C)n3C)cc2)[C@@H](C)C(=O)N[C@@H](COC)C(=O)N(C)[C@@H](Cc2ccc(Cl)cc2)CC(=O)N[C@H]2CCCC[C@@H]2N(C)C1=O] | Compound 147 (Example 2.4) | Analytical method 4 t_{R} = 2.11 min [M + H]⁺ = 987.2 |
| 290 | 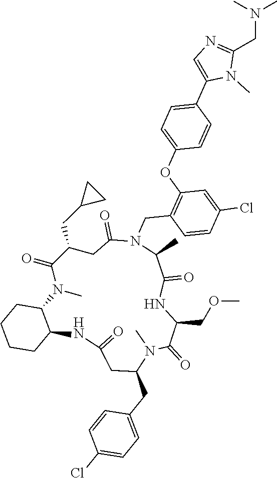 [SMILES: COC[C@@H]1NC(=O)[C@H](C)N(Cc2ccc(Cl)cc2Oc2ccc(-c3cnc(CN(C)C)n3C)cc2)C(=O)C[C@@H](CC2CC2)C(=O)N(C)[C@H]2CCCC[C@@H]2NC(=O)C[C@H](Cc2ccc(Cl)cc2)N(C)C1=O] | Compound 147 (Example 2.4) | Analytical method 4 t_{R} = 2.12 min [M + H]⁺ = 1001.2 |

### Example 2.5: Method E—Synthesis of 4S,7S,10S,14R)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxy-methyl)-5,10,16-trimethyltetradecahydro-1H-pyrano[3,4-1][1,4,7,11,14]pentaazacyclooctadecine-2,6,9,12,15(3H)-pentaone trifluoroacetate (Compound 61)

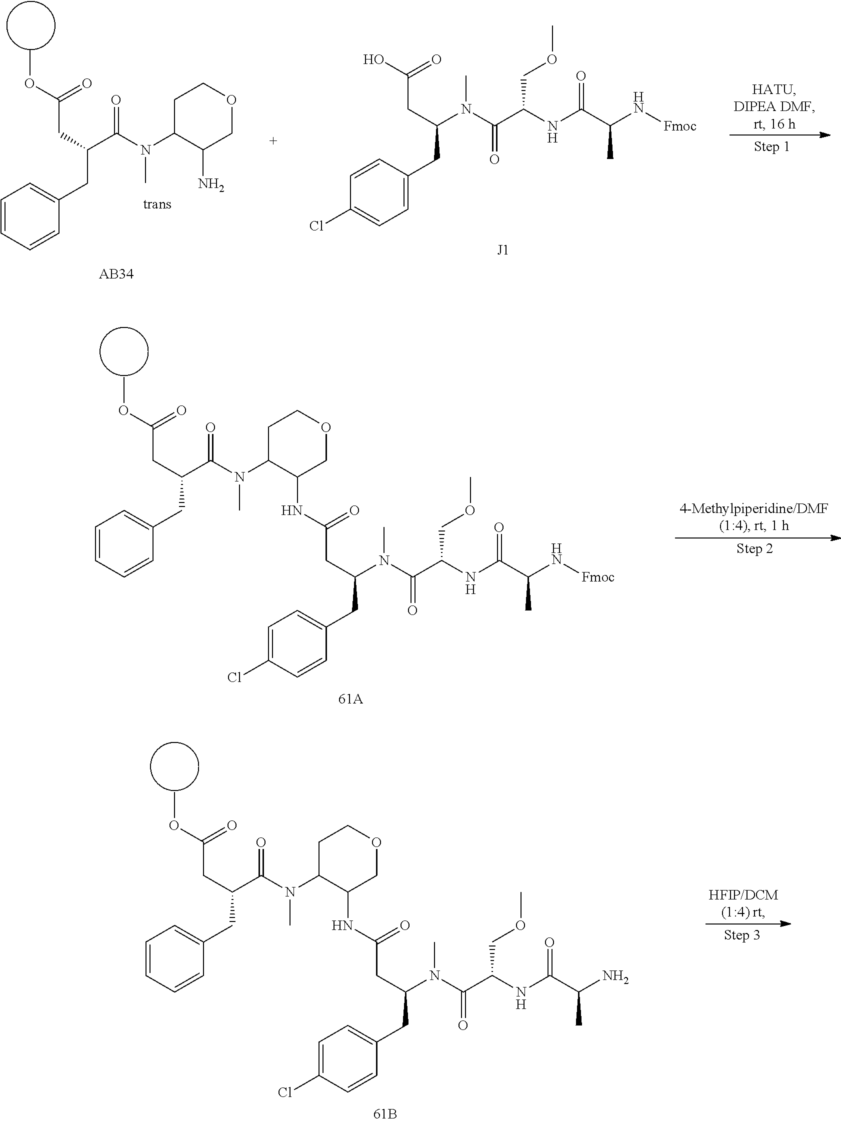

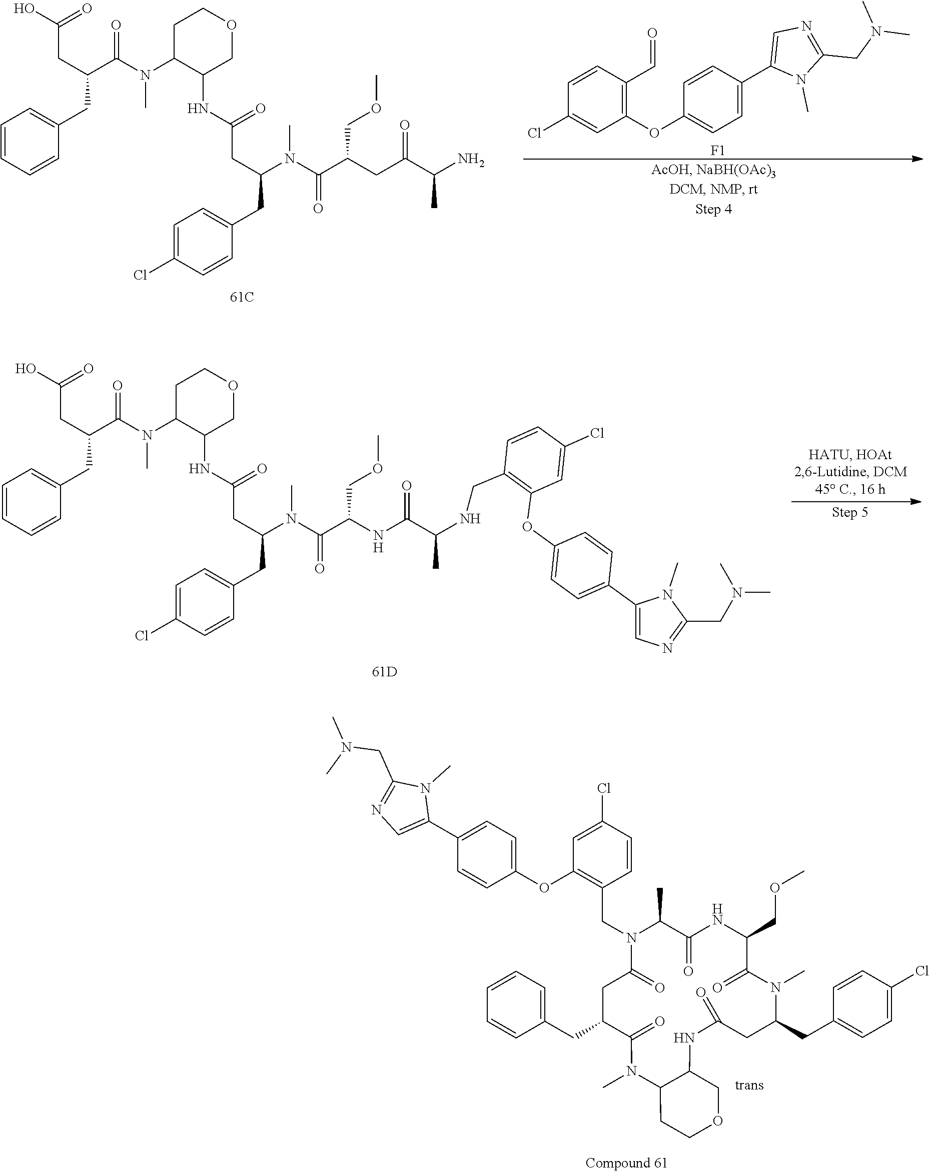

### Step 1. 2-chloro-trityl Resin Bound—(3R)-3-benzyl-4-((3-((5S,8S,11S)-11-(4-chlorobenzyl)-1-(9H-fluoren-9-yl)-8-(methoxymethyl)-5,10-dimethyl-3,6,9-trioxo-2-oxa-4,7,10-triazatridecan-13-amido)tetrahydro-2H-pyran-4-yl)(methyl)amino)-4-oxobutanoic acid (61A)

Intermediate 61A was prepared according to the procedure used in Example 2.3, Step 1 starting from resin AB34 (trans racemic) (52 mg, 0.085 mmol) and intermediate J1 (106 mg, 0.17 mmol). 61A (103 mg, crude) was carried onto the next step without purification.

### Step 2. 2-Chloro trityl Resin Bound—(3R)-4-((3-((S)-3-((S)-2-((S)-2-aminopropanamido)-3-methoxy-N-methyl propanamido)-4-(4-chlorophenyl)butanamido)tetrahydro-2H-pyran-4-yl)(methyl)amino)-3-benzyl-4-oxobutanoic acid (61B)

Intermediate 61B was prepared according to the procedure used in Example 2.1, Step 1-2 starting from 61A (103 mg, 0.085 mmol). 61B (84 mg, crude) was carried onto the next step without purification.

### Step 3. (3R)-4-((3-((S)-3-((S)-2-((S)-2-aminopropanamido)-3-methoxy-N-methylpropanamido)-4-(4-chloro phenyl)butanamido)tetrahydro-2H-pyran-4-yl)(methyl)amino)-3-benzyl-4-oxobutanoic acid (61C)

Intermediate 61C (20 mg, 34%) was prepared according to the procedure used in Example 2.3, Step 2 starting from 61B (84 mg, 0.085 mmol). Analytical method 7; t_{R}=0.74 min; [M+H]⁺=702.5.

### Step 4. (3R)-3-benzyl-4-((3-((S)-3-((S)-2-((S)-2-((4-chloro-2-(4-(2-((dimethylamino)methyl)-1-methyl-1H-imidazol-5-yl)phenoxy)benzyl)amino)propanamido)-3-methoxy-N-methylpropanamido)-4-(4-chloro phenyl)butanamido)tetrahydro-2H-pyran-4-yl)(methyl)amino)-4-oxobutanoic acid (61)

Intermediate 61D (5 mg, 17%) was prepared according to the procedure used in Example 2.1, Step 5 starting from intermediate 61C (20 mg, 0.028 mmol) and intermediate F1 (10.5 mg, 0.028 mmol). Analytical method 7; t_{R}=0.89 min; [M+H]⁺=1055.5.

### Step 5. (4S,7S,10S,14R)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyltetradecahydro-1H-pyrano[3,4-1][1,4,7,11,14]pentaazacyclooctadecine-2,6,9,12,15(3H)-pentaone (Compound 61)

Compound 61 was prepared according to the procedure used in Example 2.3, Step 3 starting from intermediate 61D (5 mg, 0.004 mmol). This provided Compound 61 as a white solid (1 mg, 17%). Analytical method 7; t_{R}=0.96 min; [M+H]⁺=1037.4.

### Example 2.6: Method F Exemplified by the Synthesis of (2S,5S,8S,13S,16R)-16-benzyl-8-(4-chlorobenzyl)-1-(4-ethyl-2-(4-(1-methyl-2-(pyrrolidin-1-ylmethyl)-1H-imidazol-5-yl)phenoxy)benzyl)-2-(2-fluoroethyl)-5-(methoxymethyl)-7,13,14-trimethyl-1,4,7,11,14-pentaazacyclooctadecane-3,6,10,15,18-pentaone (Compound 26)

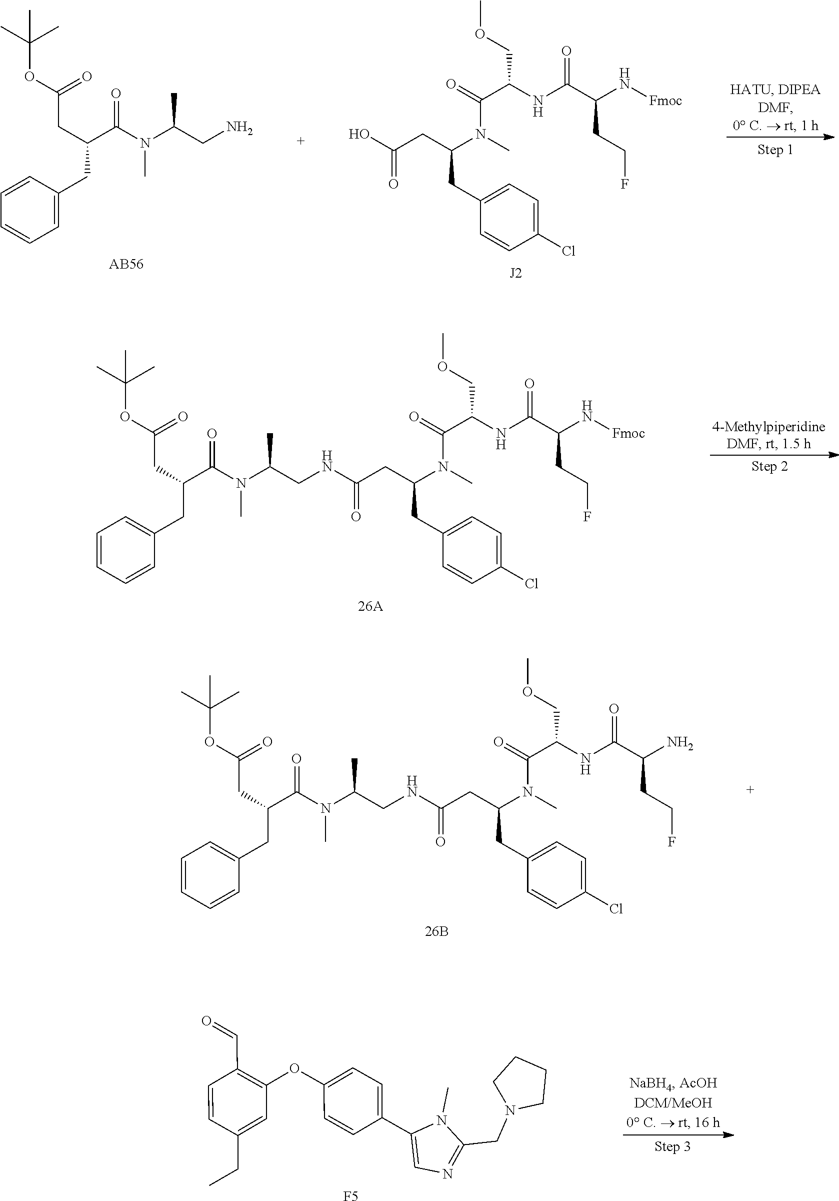

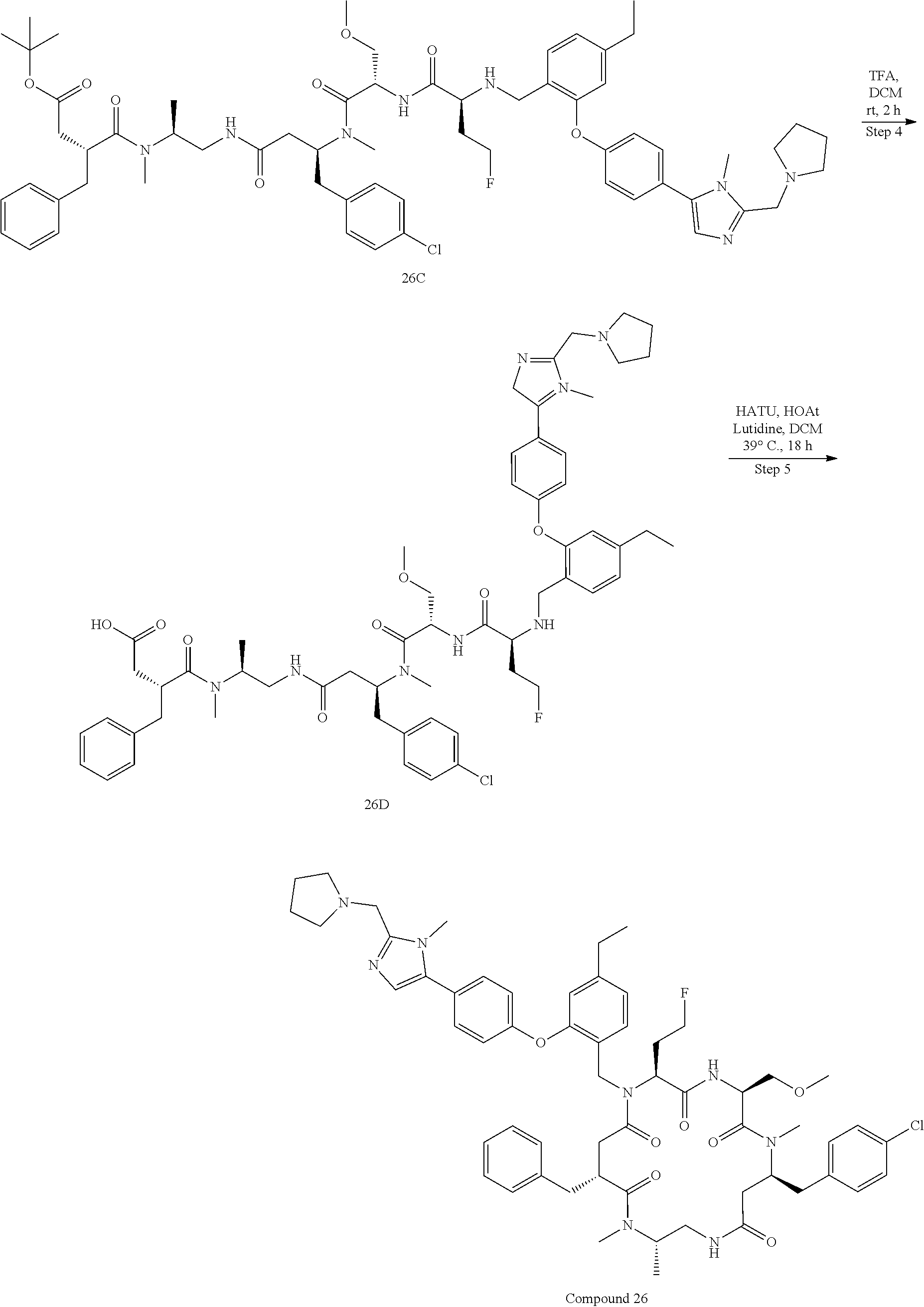

### Step 1. (5S,8S,11S,16S,19R)-tert-Butyl 19-benzyl-11-(4-chlorobenzyl)-1-(9H-fluoren-9-yl)-5-(2-fluoroethyl)-8-(methoxymethyl)-10,16,17-trimethyl-3,6,9,13,18-pentaoxo-2-oxa-4,7,10,14,17-pentaazahenicosan-21-oate, (5S,8S,11S,16S,19R)-tert-butyl 19-benzyl-11-(4-chlorobenzyl)-5-(2-fluoroethyl)-8-(methoxymethyl)-10,16,17-trimethyl-3,6,9,13,18-pentaoxo-4,7,10,14,17-pentaazahenicosan-21-oate (26A)

To a solution of J2 (200 mg, 0.306 mmol) in DMF (3 mL) at 0° C. was added DIPEA (0.160 mL, 0.917 mmol) and HATU (122 mg, 0.321 mmol). The mixture was stirred at room temperature for 5 minutes and then AB56 (102 mg, 0.306 mmol) was added. The resulting mixture was stirred at room temperature for 1 hour. The crude product was purified by normal phase chromatography (40 g silica-gel column, eluting with 70-100% EtOAc/Heptane) to provide 26A (291 mg, crude) as a yellow oil that was used directly in the next step without purification. Analytical method 5; t_{R}=1.33 min; [M+H]⁺=970.3.

### Step 2. (3R,6S,11S,14S,17S)-tert-butyl 17-amino-3-benzyl-11-(4-chlorobenzyl)-19-fluoro-14-(methoxymethyl)-5,6,12-trimethyl-4,9,13,16-tetraoxo-5,8,12,15-tetraazanonadecan-1-oate, 1-((9H-fluoren-9-yl)methyl)-4-methylpiperidine (26B)

To a solution of 26A (291 mg, 0.3 mmol) in DMF (2 mL) was added 4-methylpiperidine (0.249 mL, 2.1 mmol) and the resulting mixture was stirred for 1.5 h at room temperature. The reaction mixture was purified by reverse-phase chromatography, using C18 column (40 g, eluting with 40-100% MeCN/water, 0.1% NH₄OH). The fractions containing the product were lyophilized to dryness to afford 26B as a white solid (30 mg, 13% yield, over two steps). Analytical method 5; t_{R}=1.33 min; [M+H]⁺=970.3.

### Step 3. (3S,6S,9S,14S,17R)-tert-butyl 17-benzyl-9-(4-chlorobenzyl)-1-(4-ethyl-2-(4-(1-methyl-2-(pyrrolidin-1-ylmethyl)-1H-imidazol-5-yl)phenoxy)phenyl)-3-(2-fluoroethyl)-6-(methoxymethyl)-8,14,15-trimethyl-4,7,11,16-tetraoxo-2,5,8,12,15-pentaazanonadecan-19-oate (26C)

A mixture of 26B (30 mg, 0.040 mmol) and aldehyde F5 (15.61 mg, 0.040 mmol) in DCM (4 mL) was stirred at room temperature overnight. The reaction mixture was concentrated to dryness and then MeOH (4 mL) was added to afford a slightly cloudy solution. To a stirred mixture cooled using an ice bath was slowly added sodium borohydride (4.55 mg, 0.120 mmol) in portions, followed by acetic acid (3.44 μL, 0.060 mmol). The resulting mixture was cooled using an ice bath and stirred for 30 minutes. Once LCMS showed complete consumption of starting materials, the reaction was quenched with 0.1 mL of water and acetic acid (3.44 μL, 0.060 mmol), warmed to room temperature, and then stirred for 30 minutes. The mixture was concentrated and the crude product was directly purified by reversed-phase chromatography, using C18 column (40 g, eluting with 40-100% MeCN/water, 0.1% NH₄OH). The fractions containing product were lyophilized to dryness to afford 26C (45 mg, 100% yield) as a white solid. Analytical method 5; t_{R}=1.38 min; [M+H]⁺=1120.8.

### Step 4. (3S,6S,9S,14S,17R)-17-benzyl-9-(4-chlorobenzyl)-1-(4-ethyl-2-(4-(1-methyl-2-(pyrrolidin-1-ylmethyl)-1H-imidazol-5-yl)phenoxy)phenyl)-3-(2-fluoroethyl)-6-(methoxymethyl)-8,14,15-trimethyl-4,7,11,16-tetraoxo-2,5,8,12,15-pentaazanonadecan-19-oic acid (26D)

To a mixture of 26C (45 mg, 0.040 mmol) in DCM (3 mL) was added trifluoroacetic acid (0.464 mL, 6.02 mmol) and the resulting mixture was stirred at room temperature for 2 hours. Another 0.15 mL trifluoroacetic acid was added and the mixture was stirred for another 1 hour. Once LCMS showed complete consumption of starting materials, the reaction mixture was concentrated to dryness and purified by reversed-phase chromatography using C18 column (40 g, eluting with 40-100% MeCN/water, 0.1% TFA). The fractions containing product were lyophilized to dryness to afford 26D (20 mg, 47% yield) as a white solid. Analytical method 5; t_{R}=0.90 min; [M+H]⁺=1066.1.

### Step 5. (2S,5S,8S,13S,16R)-16-benzyl-8-(4-chlorobenzyl)-1-(4-ethyl-2-(4-(1-methyl-2-(pyrrolidin-1-ylmethyl)-1H-imidazol-5-yl)phenoxy)benzyl)-2-(2-fluoroethyl)-5-(methoxymethyl)-7,13,14-trimethyl-1,4,7,11,14-pentaazacyclooctadecane-3,6,10,15,18-pentaone (Compound 26)

A mixture of 26D (20 mg, 0.019 mmol), HATU (28.5 mg, 0.075 mmol) and HOAt (3.83 mg, 0.028 mmol) in DCM (80 mL) was stirred for 2 minutes at room temperature. 2,6-Lutidine (0.044 mL, 0.375 mmol) was then added and the resulting mixture was stirred for 18 hours at 39° C. The reaction mixture was quenched with water (5 mL), and the organic phase was separated and then concentrated under reduced pressure without heat. The crude product was first purified with reverse-phase chromatography using C18 column (40 g, eluting with 40-100% MeCN/water, 0.1% TFA). The fractions containing product were lyophilized to dryness and then repurified using basic reverse-phase chromatography using C18 column (40 g, eluting with 40-100% MeCN/water, 0.1% NH₄OH). The fractions containing product were lyophilized to dryness to afford Compound 26 (2 mg, 47% yield) as a white solid. Analytical method 5; t_{R}=1.24 min; [M+H]⁺=1047.5.

### Example 2.7: Method G—Synthesis of (2S,5S,8S,13S,16R)-16-allyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaaza cyclooctadecane-3,6,10,15,18-pentaone (Compound 44)

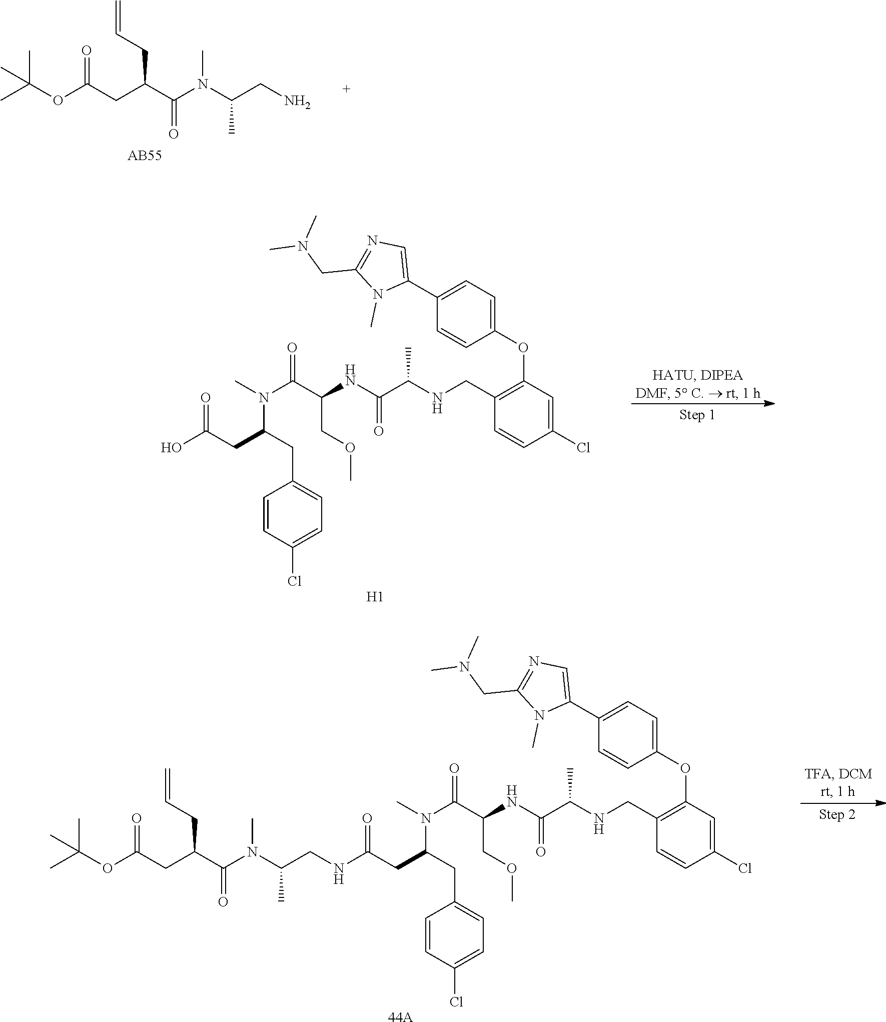 [SMILES: C=CC[C@H](CC(=O)OC(C)(C)C)C(=O)N(C)[C@@H](C)CN ; COC[C@H](NC(=O)[C@H](C)NCc1ccc(Cl)cc1Oc1ccc(-c2cnc(CN(C)C)n2C)cc1)C(=O)N(C)[C@H](CC(=O)O)Cc1ccc(Cl)cc1 ; "H1" ; C=CC[C@H](CC(=O)OC(C)(C)C)C(=O)N(C)[C@@H](C)CNC(=O)C[C@H](Cc1ccc(Cl)cc1)N(C)C(=O)[C@H](COC)NC(=O)[C@H](C)NCc1ccc(Cl)cc1Oc1ccc(-c2cnc(CN(C)C)n2C)cc1 ; "44A"]

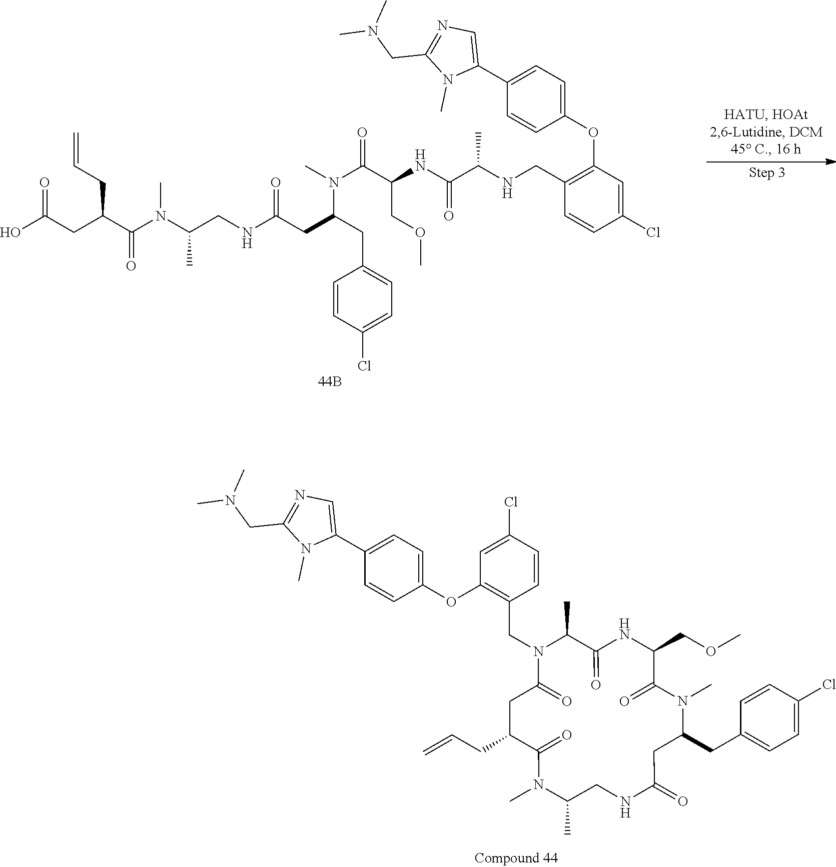

### Step 1. tert-butyl (3S,6S,9S,14S,17R)-17-allyl-1-(4-chloro-2-(4-(2-((dimethylamino)methyl)-1-methyl-1H-imidazol-5-yl)phenoxy)phenyl)-9-(4-chlorobenzyl)-6-(methoxymethyl)-3,8,14,15-tetramethyl-4,7,11,16-tetraoxo-2,5,8,12,15-pentaazanonadecan-19-oate (44A)

To a solution of intermediate H1 (200 mg, 0.265 mmol) in DMF (3 mL) at 0° C. was added DIPEA (0.463 mL, 2.65 mmol) and HATU (111 mg, 0.292 mmol). After 5 min, intermediate AB55 (83 mg, 0.292 mmol) was added and the resulting mixture was stirred at room temperature for 1 h. The reaction mixture was concentrated and the crude product was purified via reversed-phase chromatography using 100 g C18 column (eluting 10-100% MeCN in water with 0.1% NH₄OH) to afford intermediate 44A (130 mg, 48.0%) as a white solid. Analytical method 4; t_{R}=1.60 min; [M+H]⁺=1019.9.

### Step 2. (3S,6S,9S,14S,17R)-17-allyl-1-(4-chloro-2-(4-(2-((dimethylamino)methyl)-1-methyl-1H-imidazol-5-yl)phenoxy)phenyl)-9-(4-chlorobenzyl)-6-(methoxymethyl)-3,8,14,15-tetramethyl-4,7,11,16-tetraoxo-2,5,8,12,15-pentaazanonadecan-19-oic acid (44B)

44A (130 mg, 0.127 mmol) in 20% TFA in DCM (10 mL) at rt was stirred for 1 h. The reaction mixture was then concentrated in vacuo and the crude residue was purified via reversed-phase chromatography (100 g 018 column, eluting with 10-100% MeCN in water with 0.1% NH₄OH) to afford intermediate 44B (70 mg, 54%) as a white solid. Analytical method 4; t_{R}=1.34 min; [M+H]⁺=963.7.

### Step 3. (25,55,85,13S,16R)-16-allyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-1,4,7,11,14-penta azacyclooctadecane-3,6,10,15,18-pentaone (Compound 44)

Compound 44 (19 mg, 26%) was prepared according to the procedure used in Example 2.3, Step 3 from 44B (70 mg, 0.073 mmol). Analytical method 7; t_{R}=0.92 min; [M+H]⁺=945.3.

The Compounds shown in Table 5 below were prepared according to the procedure used for Example 2.7, Compound 44 (Example 2.7) from the respective intermediates shown in Table 20 and Table 32 herein below.

TABLE 5

| Cmd | | Synthesis | |
|---|---|---|---|
| No. | Structure | Procedure | LCMS |
| 2 | 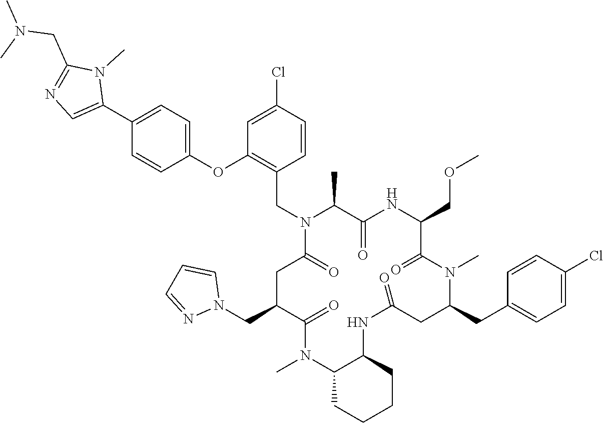 [SMILES: COC[C@@H]1NC(=O)[C@H](C)N(Cc2ccc(Cl)cc2Oc2ccc(-c3cnc(CN(C)C)n3C)cc2)C(=O)C[C@H](Cn2cccn2)C(=O)N(C)[C@H]2CCCC[C@@H]2NC(=O)C[C@H](Cc2ccc(Cl)cc2)N(C)C1=O] | Compound 44 (Example 2.7) | Analytical method 2 t_{R} = 2.71 min [M + H]⁺ = 1025.6 |
| 18 | 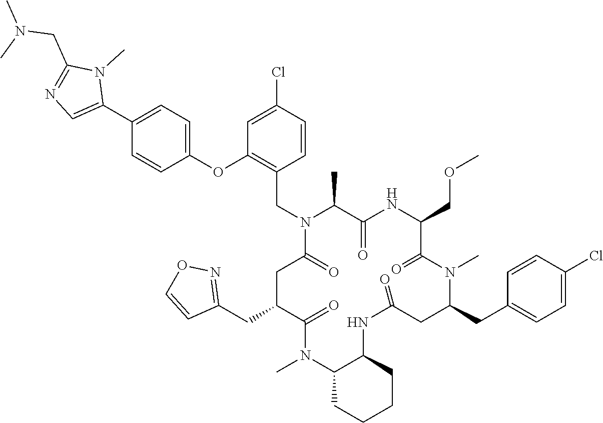 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccon2)C(=O)N(C)[C@H]2CCCC[C@@H]2NC(=O)C[C@H](Cc2ccc(Cl)cc2)N(C)C1=O] | Compound 44 (Example 2.7) | Analytical method 5 t_{R} = 1.11 min [M + H]⁺ = 1026.9 |
| 33 | 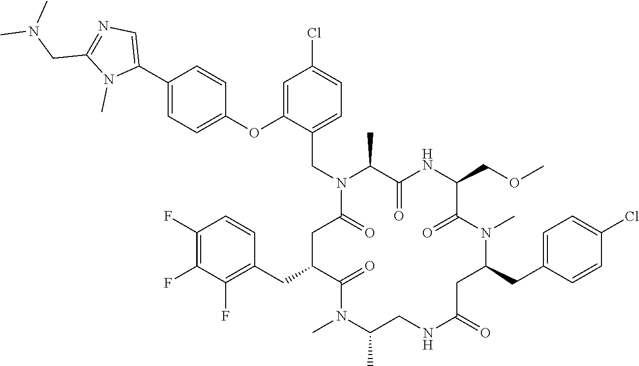 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccc(F)c(F)c2F)C(=O)N(C)[C@@H](C)CNC(=O)C[C@H](Cc2ccc(Cl)cc2)N(C)C1=O] | Compound 44 (Example 2.7) | Analytical method 5 t_{R} = 1.17 min [M + H]⁺ = 1049.6 |
| 56 | 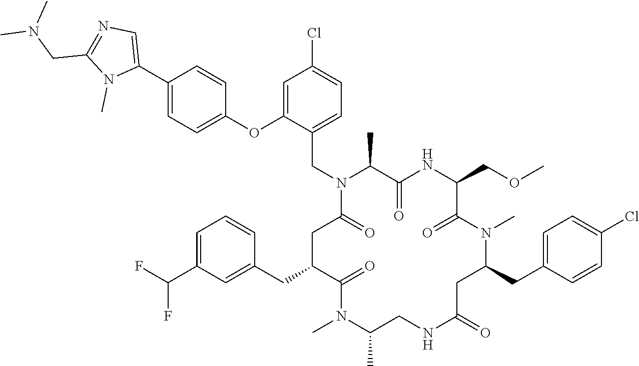 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2cccc(C(F)F)c2)C(=O)N(C)[C@@H](C)CNC(=O)C[C@H](Cc2ccc(Cl)cc2)N(C)C1=O] | Compound 44 (Example 2.7) | Analytical method 5 t_{R} = 1.18 min [M + H]⁺ = 1045.3 |
| 57 | 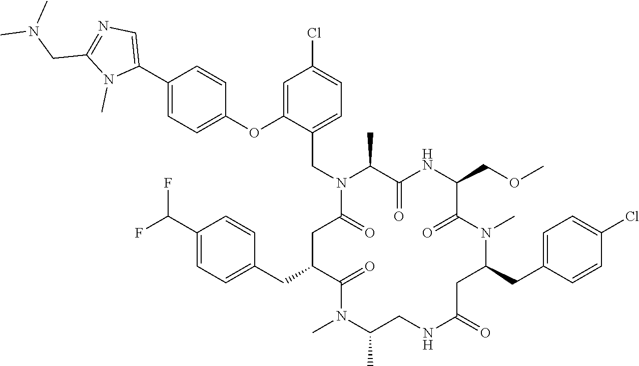 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccc(C(F)F)cc2)C(=O)N(C)[C@@H](C)CNC(=O)C[C@H](Cc2ccc(Cl)cc2)N(C)C1=O] | Compound 44 (Example 2.7) | Analytical method 5 t_{R} = 1.18 min [M + H]⁺ = 1045.7 |
| 98 | 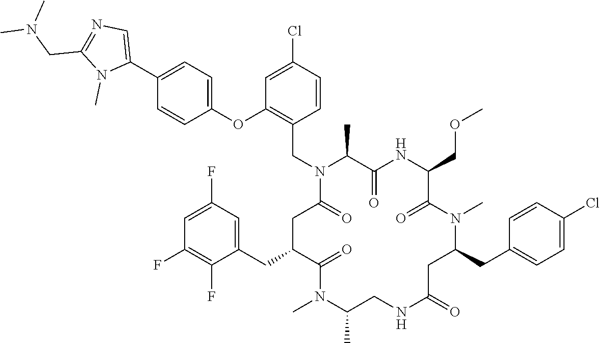 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2cc(F)cc(F)c2F)C(=O)N(C)[C@@H](C)CNC(=O)C[C@H](Cc2ccc(Cl)cc2)N(C)C1=O] | Compound 44 (Example 2.7) | Analytical method t_{R} = 2.07 min [M + H]⁺ = 1049.0 |
| 99 | 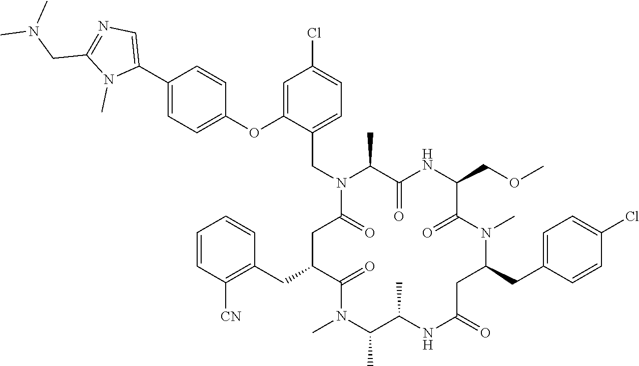 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2C#N)C(=O)N(C)[C@@H](C)[C@H](C)NC(=O)C[C@H](Cc2ccc(Cl)cc2)N(C)C1=O] | Compound 44 (Example 2.7) | Analytical method 5 t_{R} = 1.13 min [M + H]⁺ = 1034.3 |
| 172 | 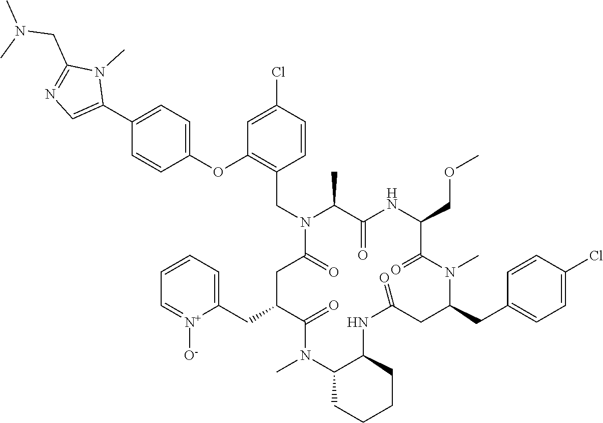 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2cccc[n+]2[O-])C(=O)N(C)[C@H]2CCCC[C@@H]2NC(=O)C[C@H](Cc2ccc(Cl)cc2)N(C)C1=O] | Compound 44 (Example 2.7) | Analytical method 7 t_{R} = 0.88 min [M + H]⁺ = 1052.2 |
| 180 | 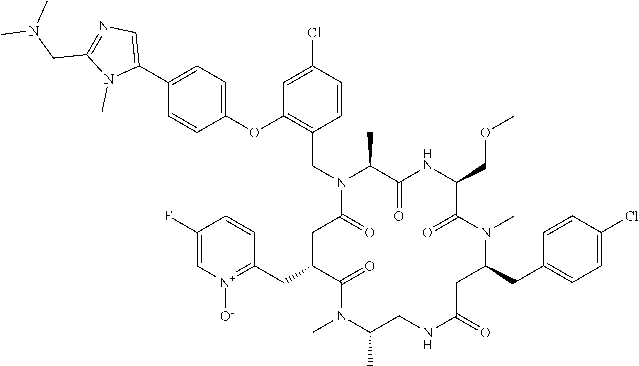 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccc(F)c[n+]2[O-])C(=O)N(C)[C@@H](C)CNC(=O)C[C@H](Cc2ccc(Cl)cc2)N(C)C1=O] | Compound 44 (Example 2.7) | Analytical method 4 t_{R} = 1.78 min [M + H]⁺ = 1032.6 |
| 238 | 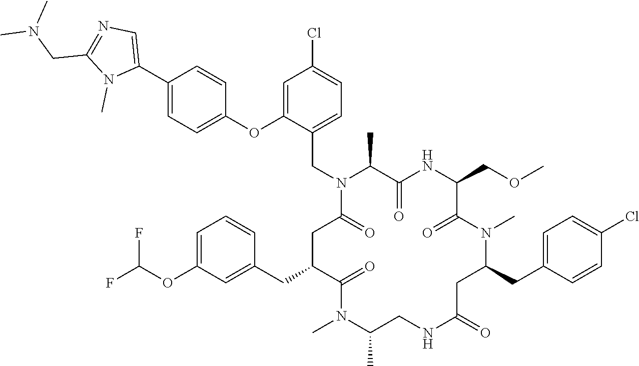 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2cccc(OC(F)F)c2)C(=O)N(C)[C@@H](C)CNC(=O)C[C@H](Cc2ccc(Cl)cc2)N(C)C1=O] | Compound 44 (Example 2.7) | Analytical method 5 t_{R} = 1.16 min [M + H]⁺ = 1061.6 |
| 246 | 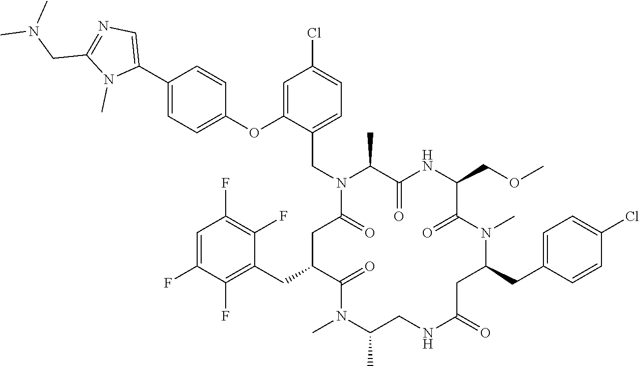 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2c(F)c(F)cc(F)c2F)C(=O)N(C)[C@@H](C)CNC(=O)C[C@H](Cc2ccc(Cl)cc2)N(C)C1=O] | Compound 44 (Example 2.7) | Analytical method 7 t_{R} = 0.95 min [M + H]⁺ = 1067.3 |
| 253 | 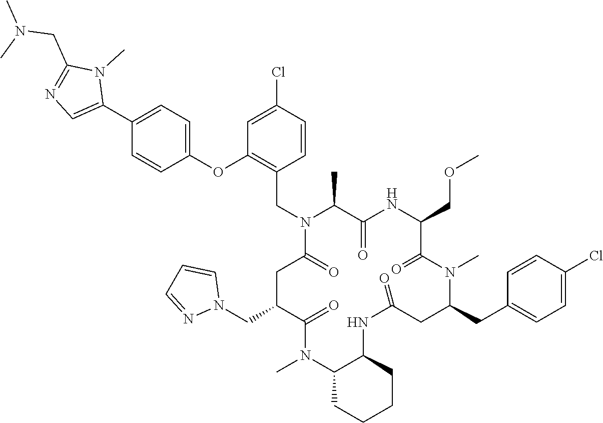 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cn2cccn2)C(=O)N(C)[C@H]2CCCC[C@@H]2NC(=O)C[C@H](Cc2ccc(Cl)cc2)N(C)C1=O] | Compound 44 (Example 2.7) | Analytical method 2 t_{R} = 2.65 min [M + H]⁺ = 1025.5 |
| 258 | 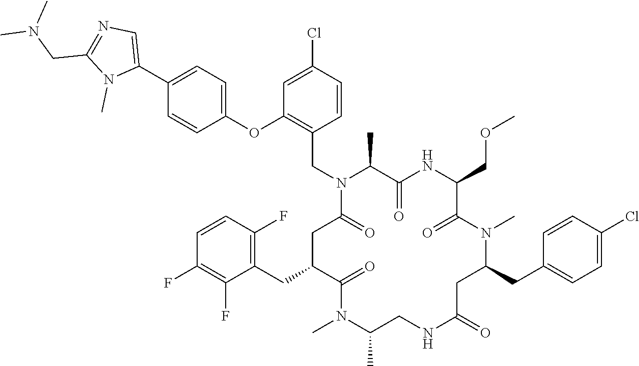 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2c(F)ccc(F)c2F)C(=O)N(C)[C@@H](C)CNC(=O)C[C@H](Cc2ccc(Cl)cc2)N(C)C1=O] | Compound 44 (Example 2.7) | Analytical method 4 t_{R} = 2.05 min [M + H]⁺ = 1049.0 |
| 261 | 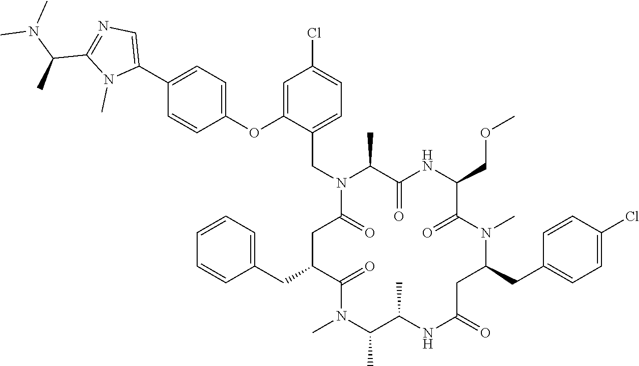 [SMILES: COC[C@@H]1NC(=O)[C@H](C)N(Cc2ccc(Cl)cc2Oc2ccc(-c3cnc([C@@H](C)N(C)C)n3C)cc2)C(=O)C[C@@H](Cc2ccccc2)C(=O)N(C)[C@@H](C)[C@H](C)NC(=O)C[C@H](Cc2ccc(Cl)cc2)N(C)C1=O] | Compound 44 (Example 2.7) | Analytical method 2 t_{R} = 2.96 min [M + H]⁺ = 1023.6 |
| 270 | 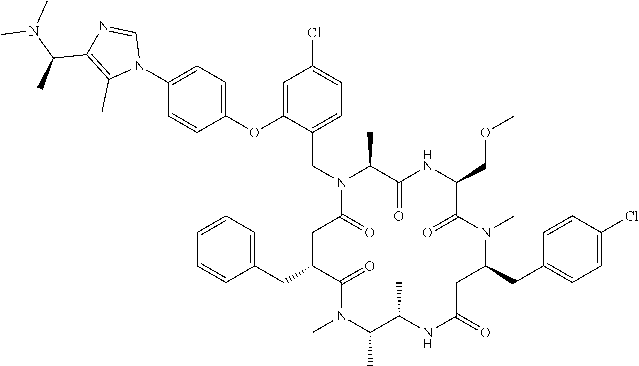 [SMILES: COC[C@@H]1NC(=O)[C@H](C)N(Cc2ccc(Cl)cc2Oc2ccc(-n3cnc([C@@H](C)N(C)C)c3C)cc2)C(=O)C[C@@H](Cc2ccccc2)C(=O)N(C)[C@@H](C)[C@H](C)NC(=O)C[C@H](Cc2ccc(Cl)cc2)N(C)C1=O] | Compound 44 (Example 2.7) | Analytical method 2 t_{R} = 3.14 min [M + H]⁺ = 1023.2 |

### Example 2.8: Method H— Synthesis of (4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-((6-methylpyridin-2-yl)methyl)hexadecahydrobenzo-[l][1,4,7,11,14]pentaazacyclooctadecine-2,6,9,12,15(3H)-pentaone (Compound 265)

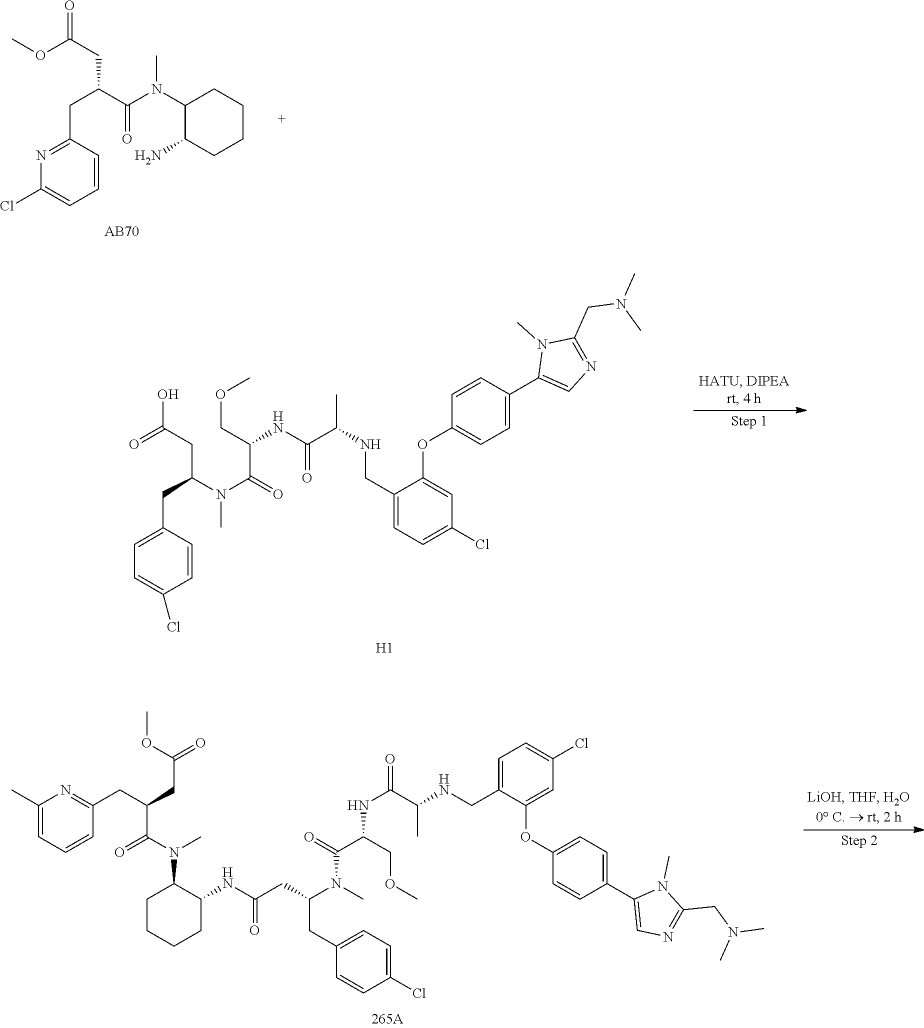

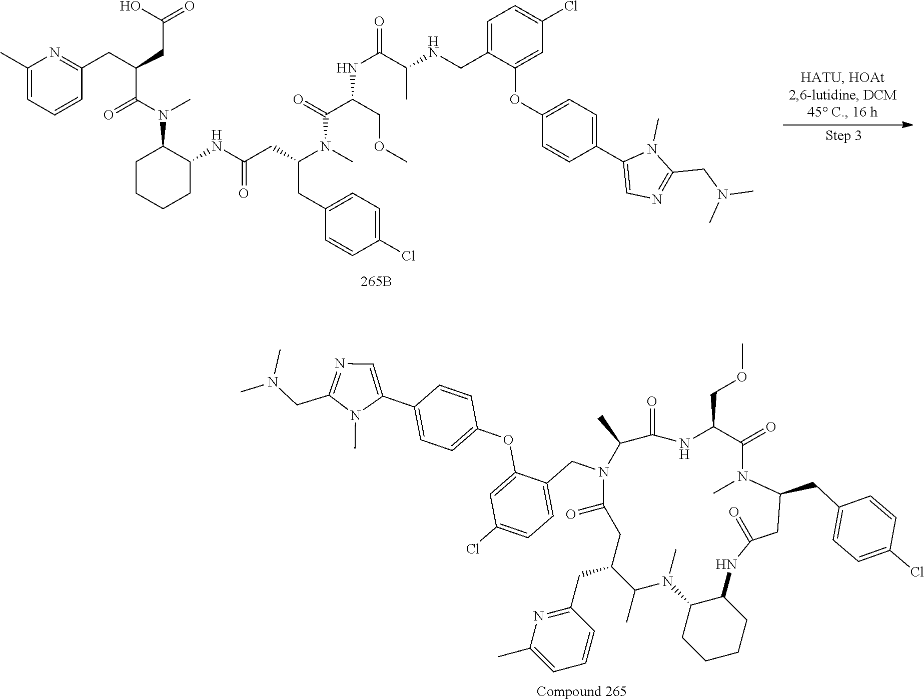

### Step 1. Methyl (R)-4-(((1S,2S)-2-((S)-3-((S)-2-((S)-2-((4-chloro-2-(4-(2-((dimethylamino)methyl)-1-methyl-1H-imidazol-5-yl)phenoxy)benzyl)amino)propanamido)-3-methoxy-N-methylpropanamido)-4-(4-chlorophenyl)-butanamido)cyclohexyl)(methyl)amino)-3-((6-methylpyridin-2-yl)methyl)-4-oxobutanoate (265A)

To a solution of AB70 (152 mg, 0.437 mmol) in DMF (6 mL) was added DIPEA (0.229 mL, 1.312 mmol), H1 (330 mg, 0.437 mmol), and HATU (183 mg, 0.481 mmol) and the resulting mixture was stirred at rt for 4 h and then concentrated. The crude material was purified by reversed-phase chromatography using (100 g C18 aq column, eluting with 10-100% MeCN in water both with 0.1% NH₄OH) to afford intermediate 265A as a white solid (254 mg, 53.6%). Analytical method 5; t_{R}=1.19 min; [M+H]⁺=1082.7.

### Step 2. (R)-4-(((1S,2S)-2-((S)-3-((S)-2-((S)-2-((4-chloro-2-(4-(2-((dimethylamino)methyl)-1-methyl-1H-imidazol-5-yl)phenoxy)benzyl)amino)propanamido)-3-methoxy-N-methylpropanamido)-4-(4-chlorophenyl)butanamido)cyclohexyl)(methyl)amino)-3-((6-methylpyridin-2-yl)methyl)-4-oxobutanoic acid (265B)

To a solution of 265A (254 mg, 0.235 mmol) in THF (8 mL) and H₂O (2 mL) was added LiOH (2 M, 0.35 mL, 0.700 mmol) at 0° C. and the resulting mixture was stirred at rt for 2 h and then diluted with EtOAc. The pH was adjusted to pH=8 with 1 N HCl and the mixture was then extracted with EtOAc. The organic layer was dried over Na₂SO₄, filtered, and concentrated to give 265B (251 mg, 100%) which was carried onto the next step without purification. Analytical method 5; t_{R}=0.83 min; [M+H]⁺=1068.8.

### Step 3. (4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-((6-methylpyridin-2-yl)-methyl)hexadecahydrobenzo[l][1,4,7,11,14]pentaazacyclooctadecine-2,6,9,12,15(3H)-pentaone (Compound 265)

Compound 265 (80 mg, 31.1%) was prepared according to the procedure used in Example 2.3, Step 3 starting from 265B (256 mg, 0.239 mmol). Analytical method 4; t_{R}=1.78 min; [M+H]⁺=1050.6.

The following Compounds shown in Table 6 below were prepared according to the procedure used for Compound 265 (Example 2.8).

TABLE 6

| Cmd | | Synthesis | |
|---|---|---|---|
| No. | Structure | Procedure | LCMS |
| 14 | 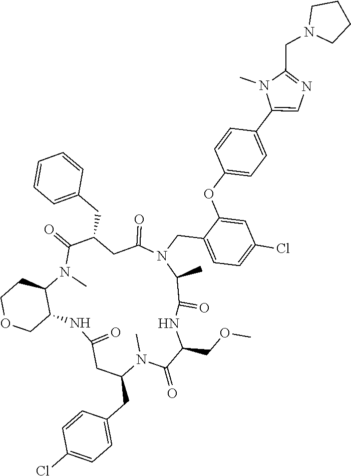 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H]2CCOC[C@H]2NC(=O)C[C@H](Cc2ccc(Cl)cc2)N(C)C1=O] | Compound 265 (Example 2.8) | Analytical method 5 t_{R} = 1.24 min [M + H]⁺ = 1062.5 |
| 65 | 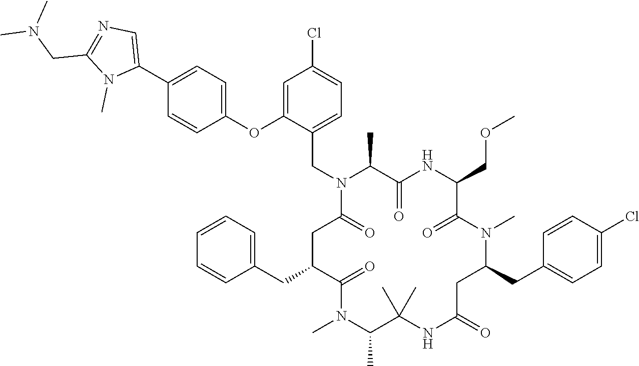 | Compound 265 (Example 2.8) | Analytical method 3 t_{R} = 1.08 min [M + H]⁺ = 1009.5 |
| 100 | 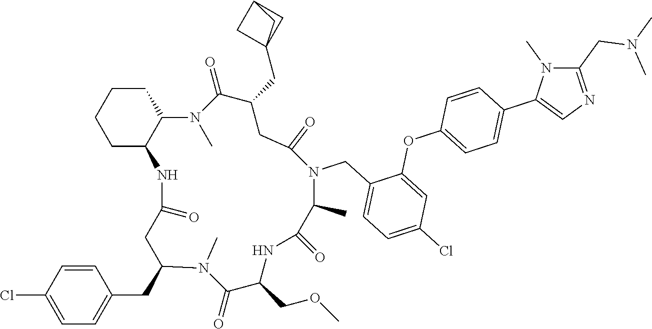 | Compound 265 (Example 2.8) | Analytical method t_{R} = 1.14 min [M + H]⁺ = 1025.5 |
| 105A | 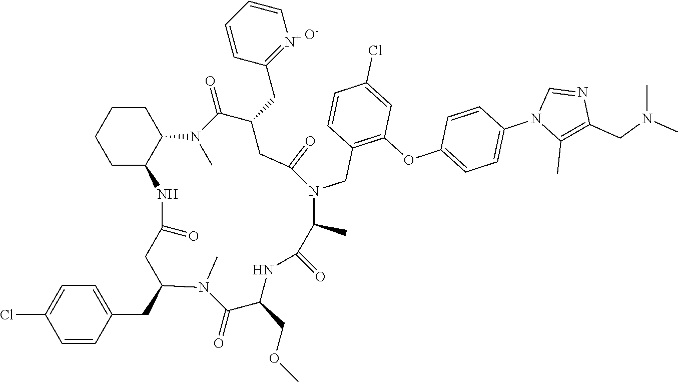 | Compound 265 (Example 2.8) | Analytical method 5 t_{R} = 1.08 min [M + H]⁺ = 1052.3 |
| 111 | 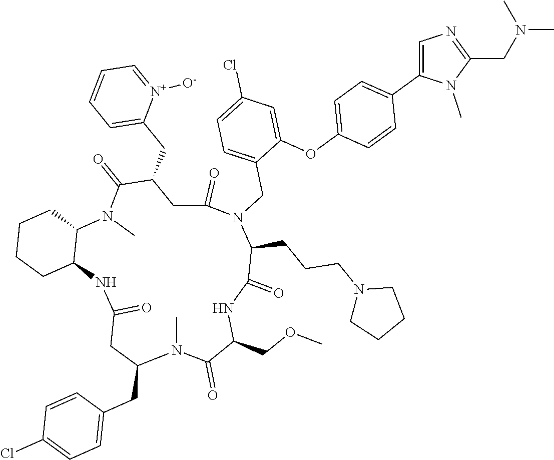 [SMILES: COC[C@@H]1NC(=O)[C@H](CCCN2CCCC2)N(Cc2ccc(Cl)cc2Oc2ccc(-c3cnc(CN(C)C)n3C)cc2)C(=O)C[C@@H](Cc2cccc[n+]2[O-])C(=O)N(C)[C@H]2CCCC[C@@H]2NC(=O)C[C@H](Cc2ccc(Cl)cc2)N(C)C1=O] | Compound 265 (Example 2.8) | Analytical method 3 t_{R} = 0.77 min [M + H]⁺ = 1149.5 |
| 187 | 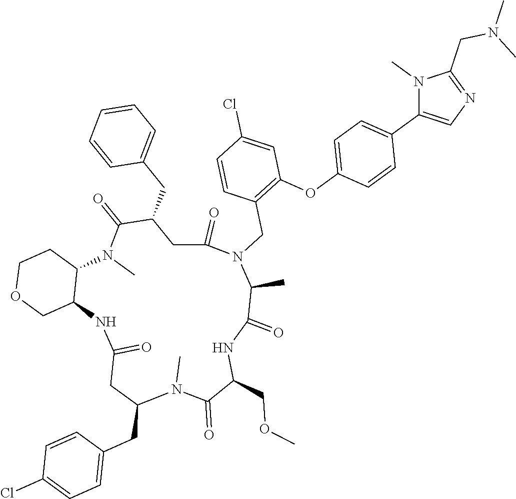 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@H]2CCOC[C@@H]2NC(=O)C[C@H](Cc2ccc(Cl)cc2)N(C)C1=O] | Compound 265 (Example 2.8) | Analytical method 7 t_{R} = 0.95 min [M + H]⁺ = 1037.4 |
| 223 | 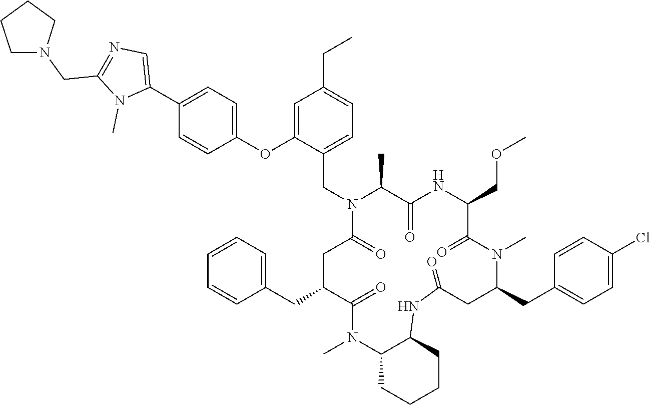 [SMILES: CCc1ccc(CN2C(=O)C[C@@H](Cc3ccccc3)C(=O)N(C)[C@H]3CCCC[C@@H]3NC(=O)C[C@H](Cc3ccc(Cl)cc3)N(C)C(=O)[C@H](COC)NC(=O)[C@@H]2C)c(Oc2ccc(-c3cnc(CN4CCCC4)n3C)cc2)c1] | Compound 265 (Example 2.8) | Analytical method 4 t_{R} = 2.22 min [M + H]⁺ = 1055.8 |
| 224 | 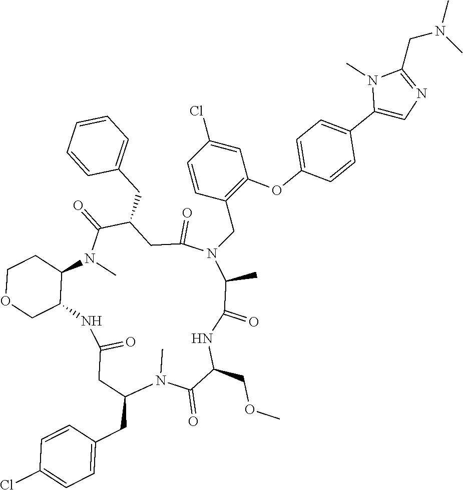 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H]2CCOC[C@H]2NC(=O)C[C@H](Cc2ccc(Cl)cc2)N(C)C1=O] | Compound 265 (Example 2.8) | Analytical method 7 t_{R} = 0.94 min [M + H]⁺ = 1037.6 |
| 227 | 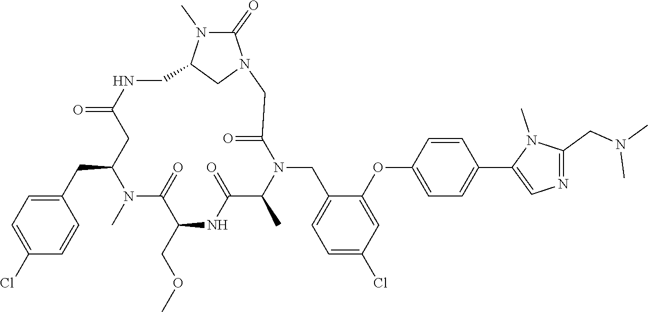 [SMILES: COC[C@@H]1NC(=O)[C@H](C)N(Cc2ccc(Cl)cc2Oc2ccc(-c3cnc(CN(C)C)n3C)cc2)C(=O)CN2C[C@H](CNC(=O)C[C@H](Cc3ccc(Cl)cc3)N(C)C1=O)N(C)C2=O] | Compound 265 (Example 2.8)- the linear peptide was synthesized as a tBu ester that was hydrolysed with TFA before the cyclization step. | Analytical method 3 t_{R} = 0.88 min [M + H]⁺ = 904.4 |
| 241 | 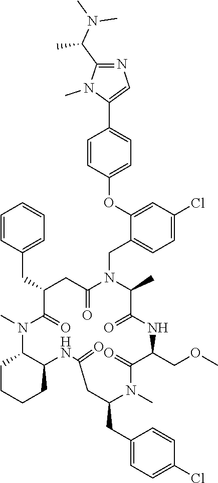 [SMILES: COC[C@@H]1NC(=O)[C@H](C)N(Cc2ccc(Cl)cc2Oc2ccc(-c3cnc([C@H](C)N(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 265 (Example 2.8) | Analytical method 4 t_{R} = 2.29 min [M + H]⁺ = 1049.6 |
| 275 | 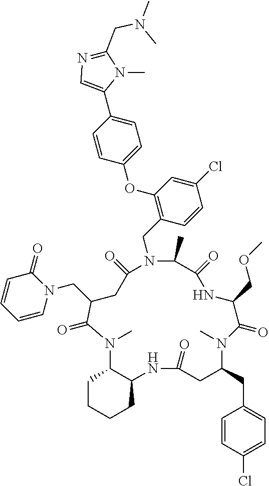 [SMILES: COC[C@@H]1NC(=O)[C@H](C)N(Cc2ccc(Cl)cc2Oc2ccc(-c3cnc(CN(C)C)n3C)cc2)C(=O)CC(Cn2ccccc2=O)C(=O)N(C)[C@H]2CCCC[C@@H]2NC(=O)C[C@H](Cc2ccc(Cl)cc2)N(C)C1=O] | Compound 265 (Example 2.8) | Analytical method 2 t_{R} = 2.48 min [M + H]⁺ = 1053.0 |
| 276 | 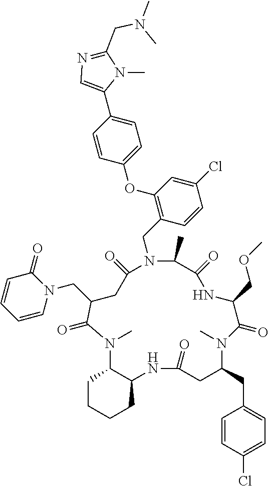 [SMILES: COC[C@@H]1NC(=O)[C@H](C)N(Cc2ccc(Cl)cc2Oc2ccc(-c3cnc(CN(C)C)n3C)cc2)C(=O)CC(Cn2ccccc2=O)C(=O)N(C)[C@H]2CCCC[C@@H]2NC(=O)C[C@H](Cc2ccc(Cl)cc2)N(C)C1=O] | Compound 265 (Example 2.8) | Analytical method 3 t_{R} = 0.98 min [M + H]⁺ = 1052.5 |
| 274 | 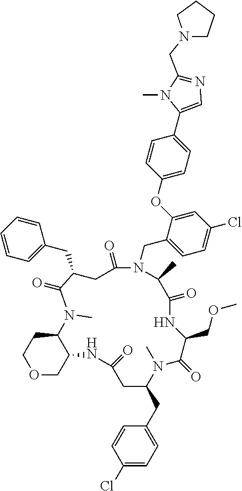 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H]2CCOC[C@H]2NC(=O)C[C@H](Cc2ccc(Cl)cc2)N(C)C1=O] | Compound 265 (Example 2.8) | Analytical method 3 t_{R} = 1.04 min [M + H]⁺ = 1063.5 |
| 279 | 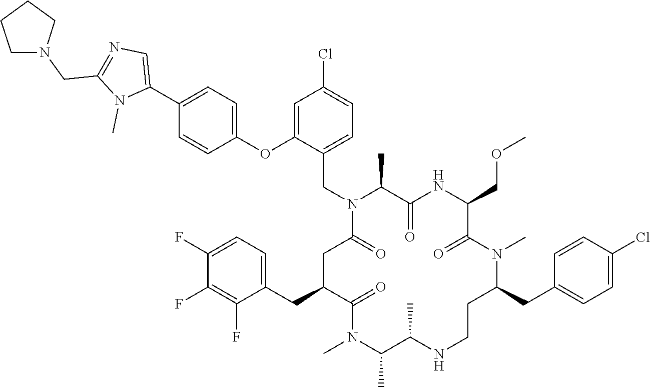 | Compound 265 (Example 2.8) | Analytical method 3 t_{R} = 1.11 min [M + H]⁺ = 1089.4 |
| 293 | 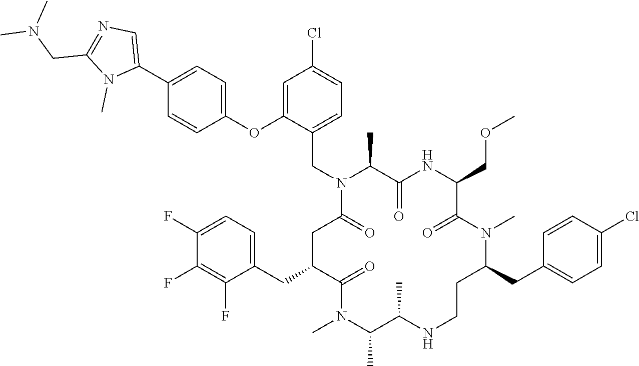 | Compound 265 (Example 2.8) | Analytical method 3 t_{R} = 1.08 min [M + H]⁺ = 1063.4 |
| 294 | 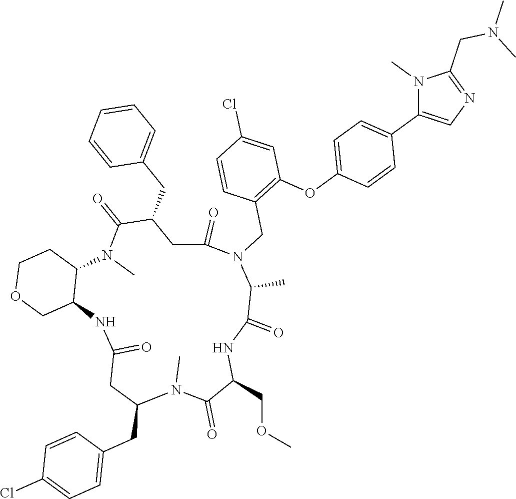 [SMILES: COC[C@@H]1NC(=O)[C@@H](C)N(Cc2ccc(Cl)cc2Oc2ccc(-c3cnc(CN(C)C)n3C)cc2)C(=O)C[C@@H](Cc2ccccc2)C(=O)N(C)[C@H]2CCOC[C@@H]2NC(=O)C[C@H](Cc2ccc(Cl)cc2)N(C)C1=O] | Compound 265 (Example 2.8)- obtained as a by- product in the synthesis of Compound 187. | Analytical method 7 t_{R} = 0.95 min [M + H]⁺ = 1038.0 |
| 295A | 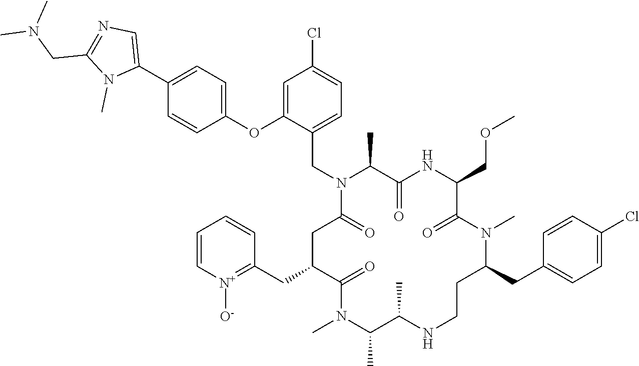 | Compound 265 (Example 2.8) | Analytical method 5 t_{R} = 100 min [M + H]⁺ = 1026.1 |

### Example 2.9: Synthesis of (4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxy-methyl)-5,16-dimethyl-10-(3-(3,3,4,4-tetrafluoropyrrolidin-1-yl)propyl)hexadecahydro-benzo[l][1,4,7,11,14]pentaazacyclooctadecine-2,6,9,12,15(3H)-pentaone (Compound 254)

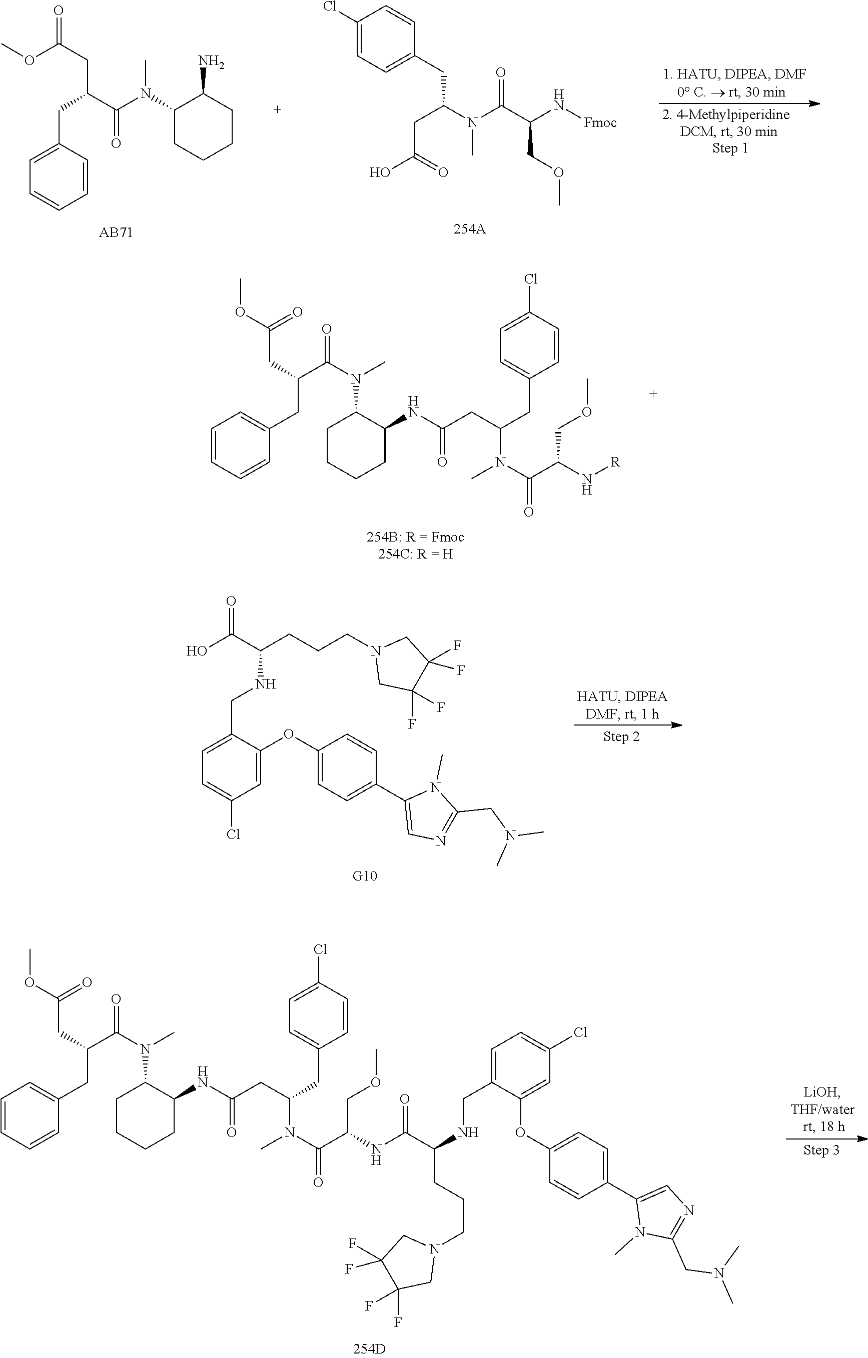

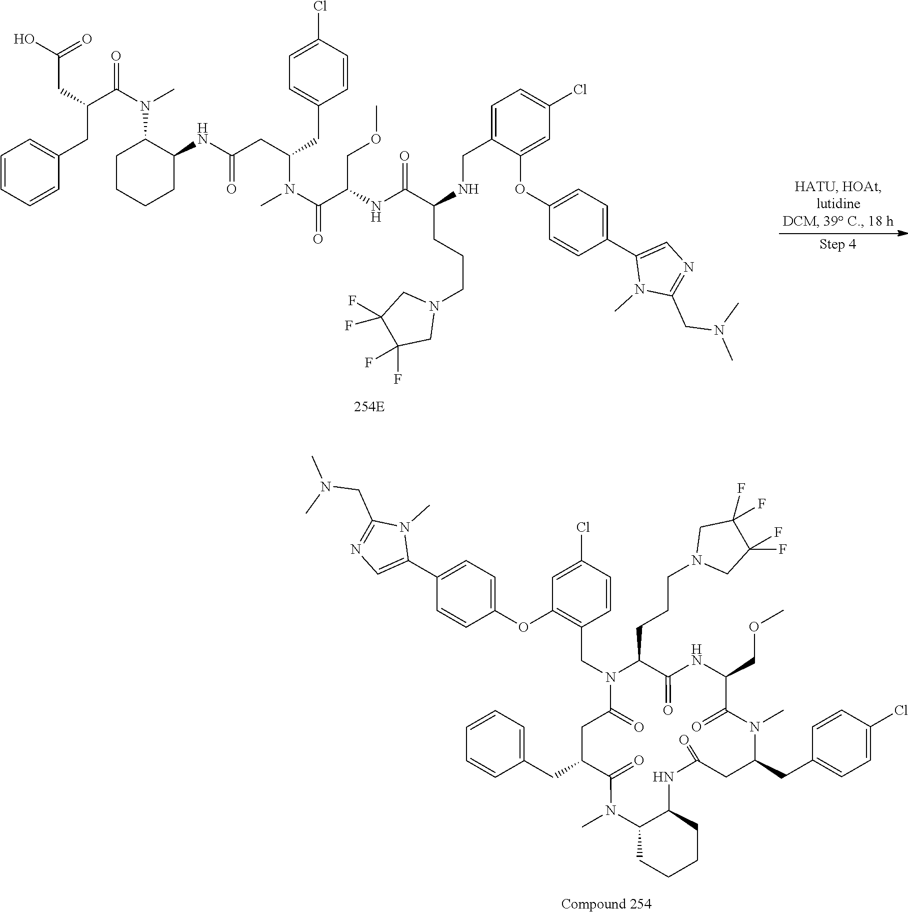

### Step 1. (R)-Methyl 4-(((1S,2S)-2-((S)-3-((S)-2-amino-3-methoxy-N-methylpropanamido)-4-(4-chlorophenyl)butanamido)cyclohexyl)(methyl)amino)-3-benzyl-4-oxobutanoate (254C)

Step 1-1: A solution of 254A (1.001 g, 1.816 mmol) (254A was obtained by cleavage of H1-2 (see Example 13.1) from the resin with HFIP), DIPEA (0.381 mL, 2.179 mmol), and HATU (0.829 g, 2.179 mmol) in DMF (7.5 mL), was stirred at 0° C. for 5 min and then a mixture of AB71 (0.67 g, 1.816 mmol) and DIPEA (0.381 mL, 2.179 mmol) in DMF (2 mL) was added. The resulting mixture was stirred at rt for 30 min. To the reaction mixture were added EtOAc (100 mL) and water (50 mL). The organic phase was separated and the organic layer was washed with brine, dried over Na₂SO₄ concentrated, and dried under high-vacuum overnight to afford 254B. The crude product was used in the next step without purification.

Step 1-2: 4-Methylpiperidine (3.22 mL, 27.2 mmol) was added to a mixture of 254B (1.572 g, 1.816 mmol) in DCM (20 mL) and the resulting mixture was stirred at rt for 30 min. The reaction mixture was concentrated and the crude product was purified by chromatography (80 g C18-column, eluting with 40-90% MeCN/water, 0.1% NH₄OH) to provide 254C (0.72 g, 62%). Analytical method 5: t_{R}=1.03 min; [M+H]⁺=643.0.

### Step 2. (R)-Methyl 3-benzyl-4-(((1S,2S)-2-((S)-3-((S)-2-((S)-2-((4-chloro-2-(4-(2-((dimethylamino)methyl)-1-methyl-1H-imidazol-5-yl)phenoxy)benzyl)amino)-5-(3,3,4,4-tetrafluoropyrrolidin-1-yl)pentanamido)-3-methoxy-N-methylpropanamido)-4-(4-chlorophenyl)butanamido)cyclohexyl)(methyl)amino)-4-oxobutanoate (254D)

To a solution of G10 (0.7 g, 0.964 mmol) in DMF (10 mL) at 0° C. was added DIPEA (1.010 mL, 5.78 mmol) and HATU (0.403 g, 1.060 mmol) and the resulting mixture was stirred at rt for 5 min. 254C (0.620 g, 0.964 mmol) was added and the resulting mixture was stirred at rt for 1 h and then purified by chromatography (80 g silica column, eluting with 30-80% DCM/MeOH, 0.1% NH₄OH) to afford 254D (928 mg, 78%). Analytical method 5: t_{R}=1.36 min; [M+H]+/2=619.2.

### Step 3. (R)-3-Benzyl-4-(((1S,2S)-2-((S)-3-((S)-2-((S)-2-((4-chloro-2-(4-(2-((dimethylamino)methyl)-1-methyl-1H-imidazol-5-yl)phenoxy)benzyl)amino)-5-(3,3,4,4-tetrafluoropyrrolidin-1-yl)pentanamido)-3-methoxy-N-methylpropanamido)-4-(4-chlorophenyl)butanamido)cyclohexyl)(methyl)amino)-4-oxobutanoic acid (254E)

A mixture of 254D (0.928 g, 0.75 mmol) in THF (30 mL) and water (3 mL) was treated with 1 M LiOH (2.25 mL, 2.25 mmol) at 0° C. and the resulting mixture was stirred at 20° C. for 16 h. Since LCMS showed starting material only, 3 mL of water and 2M LiOH aq. solution (0.7 mL) were added at 0° C. and the resulting mixture was stirred at rt for 2 h. Once LCMS showed complete consumption of starting material, the reaction mixture was cooled to 0° C. and 1.0 N HCl was added to adjust to pH 7. The organic phase removed under reduced pressure and the crude product was purified by chromatography (100 g C18 column, eluting with 5-60% MeCN/Water, 0.1% NH₄OH). The fractions containing product (e.g., MS=1223) were freeze-dried to afford 254E (0.36 g, 39%). Analytical method 5: tR=0.92 min; [M+H]+/2=611.5.

### Step 4. (4S,7S,10S,14R,16aS,20aS)-14-Benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(3-(3,3,4,4-tetrafluoropyrrolidin-1-yl)propyl)hexadecahydrobenzo[l][1,4,7,11,14]pentaazacyclooctadecine-2,6,9,12,15(3H)-pentaone (Compound 254)

To a round bottom flask containing 254E (320 mg, 0.262 mmol), HATU (398 mg, 1.046 mmol) and HOAt (53.4 mg, 0.392 mmol) was added DCM (500 mL) and the resulting mixture was stirred for 5 min at rt. 2,6-lutidine (0.914 mL, 7.85 mmol) was then added and reaction mixture was stirred for 18 h at 39° C. Water (100 mL) was then added and separated. The organic phase was evaporated under reduced pressure and the crude product was purified via chromatography (24 g C18-column, eluting with 50-100% IPA/water with 0.1% NH₄OH). The fractions containing product were freeze-dried and repurified by HPLC (eluting with MeCN/Water with 0.1% NH₄OH) to afford Compound 254 (55 mg, 17%). Analytical method 4: t_{R}=2.41 min; [M+H]⁺=1204.5.

### Example 2.10: Method K— Synthesis of 2-(((4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-10-(2-(dimethylamino)ethyl)-7-(methoxymethyl)-5,16-dimethyl-2,6,9,12,15-pentaoxodocosahydro-benzo[l][1,4,7,11,14]pentaazacyclooctadecin-14-yl)methyl)pyridine 1-oxide trifluoroacetate (Intermediate 250I)

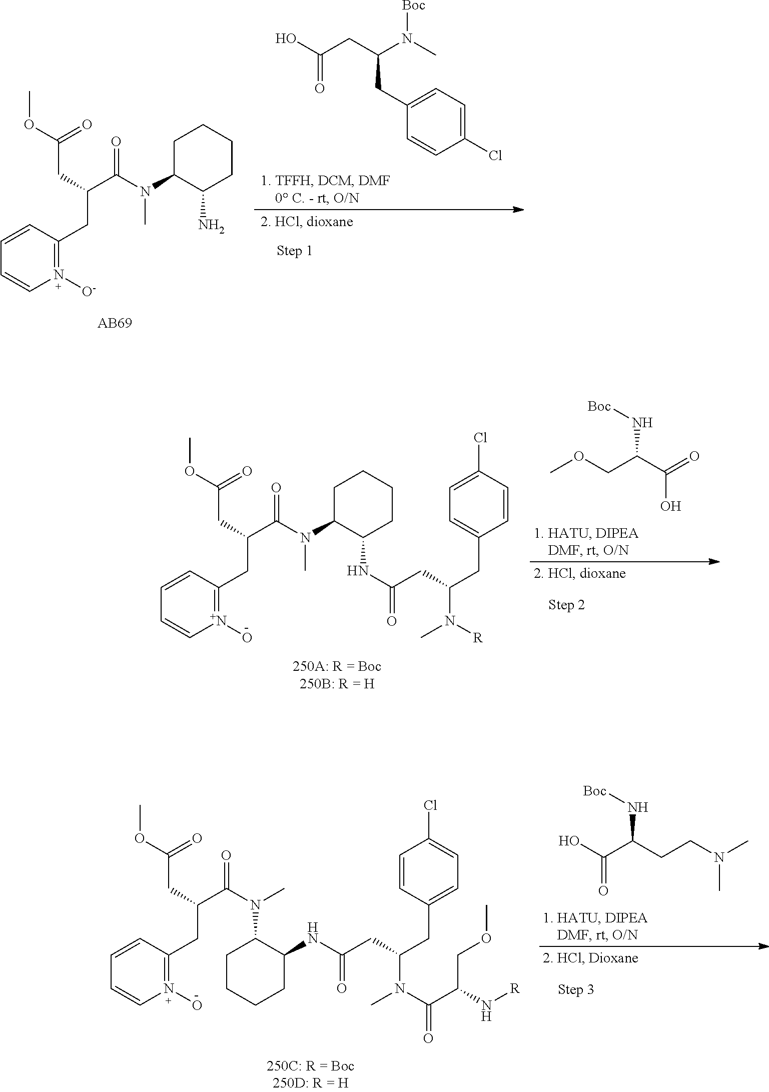

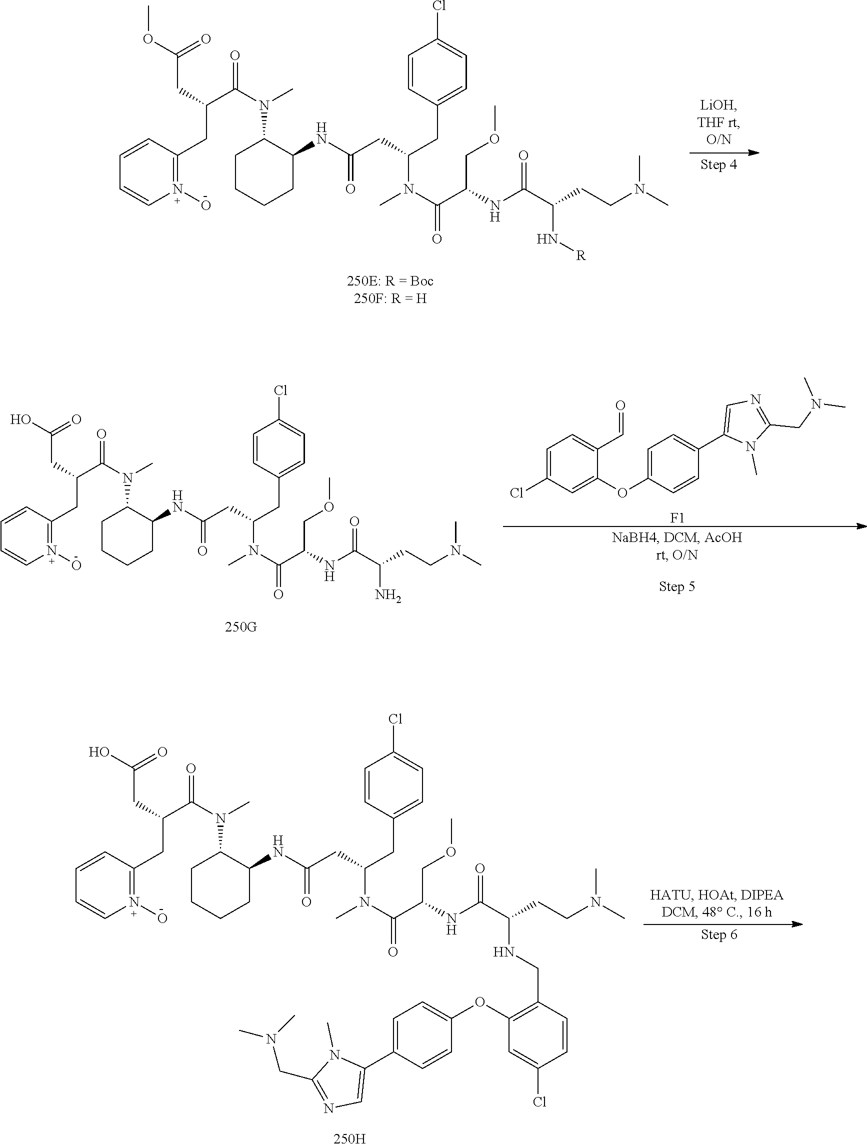

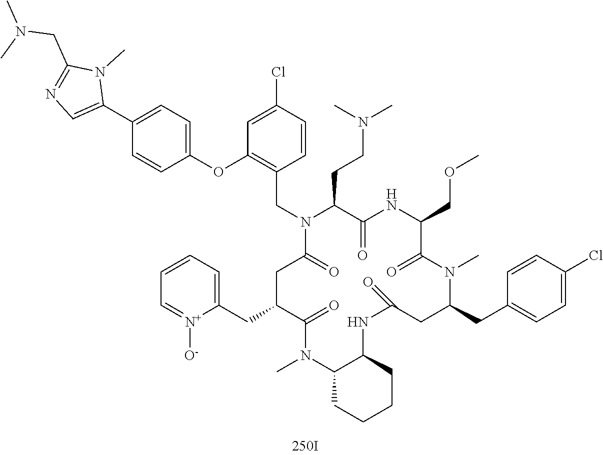 [SMILES: COC[C@@H]1NC(=O)[C@H](CCN(C)C)N(Cc2ccc(Cl)cc2Oc2ccc(-c3cnc(CN(C)C)n3C)cc2)C(=O)C[C@@H](Cc2cccc[n+]2[O-])C(=O)N(C)[C@H]2CCCC[C@@H]2NC(=O)C[C@H](Cc2ccc(Cl)cc2)N(C)C1=O]

### Step 1. 2-((R)-2-(((1S,2S)-2-((S)-4-(4-Chlorophenyl)-3-(methylamino)butanamido)cyclohexyl) (methyl)carbamoyl)-4-methoxy-4-oxobutyl)pyridine 1-oxide (250B)

Step 1-1: To a solution of AB69 (602 mg, 1.56 mmol) in DCM (30 mL)/DMF (2 mL) was added DIPEA (0.817 mL, 4.68 mmol) and TFFH (538 mg, 2.028 mmol) at 0° C. The resulting mixture was stirred for 30 min and (S)-3-((tert-butoxycarbonyl)(methyl)amino)-4-(4-chlorophenyl)butanoic acid (563 mg, 1.716 mmol) was then added. The reaction mixture was warmed to rt slowly and stirred overnight. The reaction mixture was then concentrated to remove DCM and the crude oil was purified via normal phase chromatography (40 g silica column, eluting with DCM/MeOH (98:2) to (85:15)) to afford 250A as a white solid (900 mg, 83% yield). Analytical method 7; t_{R}=1.02 min; [M+H]⁺=659.3.

Step 1-2: To 250 A (660 mg, 1.001 mmol) dissolved in dioxane (12 mL) at 0° C. was slowly added dropwise 4 M HCl in dioxane (4 mL, 16 mmol) and the resulting mixture was stirred at rt for 16 h. The reaction mixture turned cloudy and was concentrated down to a white solid and dried over vacuum to afford compound 250B (595 mg, 100% yield) which was carried onto the next step without purification. Analytical method 7; t_{R}=0.85 min; [M+H]⁺=559.0.

### Step 2. 2-((R)-2-(((1S,2S)-2-((S)-3-((S)-2-Amino-3-methoxy-N-methylpropanamido)-4-(4-chlorophenyl)-butanamido)cyclohexyl)(methyl)carbamoyl)-4-methoxy-4-oxobutyl)pyridine 1-oxide (250D)

Step 2-1: To a solution of 250B (650 mg, 1.091 mmol) in DCM (40 mL)/DMF (4 mL) was added Boc-(MeO)-Ser-OH (287 mg, 1.310 mmol) and DIPEA (0.572 mL, 3.27 mmol) at 0° C. The resulting mixture was stirred for 30 min (solid dissolved in 10 min) and then HATU (477 mg, 1.255 mmol) was added. The reaction mixture was warmed to RT slowly and stirred at room temperature overnight. The mixture was concentrated to remove DCM and the crude product was purified via silica gel chromatography (40 g column, eluting with 2-10% MeOH/DCM) to provide 250C (780 mg, 89%) as white solid. Analytical method 7; t_{R}=0.99 min; [M+H]⁺=760.2.

Step 2-2: 250D (641 mg, 100% yield, white solid) was prepared according to the procedure used in Step 1-2 starting from 250C (700 mg, 0.921 mmol). Analytical method 7; t_{R}=0.81 min; [M+H]⁺=659.7.

### Step 3. 2-((R)-2-(((1S,2S)-2-((S)-3-((S)-2-((S)-2-Amino-4-(dimethylamino)butanamido)-3-methoxy-N-methylpropanamido)-4-(4-chlorophenyl)butanamido)cyclohexyl)(methyl)carbamoyl)-4-methoxy-4-oxobutyl)pyridine 1-oxide (250F)

Step 3-1: 250E (480 mg, 72% yield, white solid) was prepared according to the procedure used in Step 2-1 from 250D (470 mg, 0.675 mmol) and (S)-2-((tert-butoxycarbonyl)amino)-4-(dimethylamino) butanoic acid (199 mg, 0.810 mmol). Analytical method 7; t_{R}=0.99 min; [(M+2)]⁺=888.6.

Step 3-2: 250F (377 mg, 94% yield, white solid) was prepared according to the procedure used in Step 2-2 from 250E (480 mg, 0.486 mmol). Analytical method 7; t_{R}=0.85 min; [M+H]⁺=788.0.

### Step 4. 2-((R)-3-(((1S,2S)-2-((S)-3-((S)-2-((S)-2-Amino-4-(dimethylamino)butanamido)-3-methoxy-N-methylpropanamido)-4-(4-chlorophenyl)butanamido)cyclohexyl)(methyl)amino)-2-(carboxymethyl)-3-oxopropyl)pyridine 1-oxide (250G)

To 250F (420 mg, 0.479 mmol) dissolved in THF (10 mL) and water (3 mL) was added 1.0 N lithium hydroxide (1.50 mL, 1.50 mmol) at 0° C. The reaction mixture was then warmed up to room temperature and stirred for 16 h. The progress of the reaction was monitored by LC/MS. The reaction mixture was cooled to 0° C., and 1.0 N HCl aqueous solution was added to adjust to pH 7. The resulting mixture was concentrated and the crude product was purified by C18 coated flash column chromatography eluting with ACN/Water with 0.1% trifluoroacetic acid. Freeze-drying of the product containing fraction afforded 250G (TFA salt, 380 mg, 89% yield). Analytical method 7; t_{R}=0.58 min; [M+H]⁺=774.1.

### Step 5. 2-((R)-2-(Carboxymethyl)-3-(((1S,2S)-2-((S)-3-((S)-2-((S)-2-((4-chloro-2-(4-(2-((dimethylamino) methyl)-1-methyl-1H-imidazol-5-yl)phenoxy)benzyl)amino)-4-(dimethylamino)butanamido)-3-methoxy-N-methylpropanamido)-4-(4-chlorophenyl)butanamido)cyclohexyl)(methyl)amino)-3-oxopropyl)pyridine 1-oxide (250H)

250H (320 mg, 94% yield, white solid), prepared according to the procedure in Example 2.6, Step 3 starting from 250G (380 mg, 0.428 mmol) and F1 (158 mg, 0.428 mmol). Analytical method 5; t_{R}=0.79 min; [M+H]⁺=1126.7.

### Step 6. 2-(((4S,7S,10S,14R,16aS,20aS)-11-(4-Chloro-2-(4-(2-((dimethylamino)methyl)-1-methyl-1H-imidazol-5-yl)phenoxy)benzyl)-4-(4-chlorobenzyl)-10-(2-(dimethylamino)ethyl)-7-(methoxymethyl)-5,16-dimethyl-2,6,9,12,15-pentaoxodocosahydrobenzo[l][1,4,7,11,14]pentaazacyclooctadecin-14-yl)methyl)pyridine 1-oxide trifluoroacetate (250I)

To a well-stirred solution of 250H (250 mg, 0.222 mmol) in DCM (200 mL), was lutidine (0.774 mL, 6.65 mmol), followed by HOAt (38.0 mg, 0.279 mmol) and the resulting mixture was stirred at rt for 5 min. HATU (337 mg 0.886 mmol) was then added in portions and the reaction mixture was warmed to 50° C. and stirred at this temperature for 16 h. The progress of the reaction was monitored by LC/MS. The reaction mixture was cooled to rt and concentrated. The crude product was purified by on a C18 coated reversed-phase 50 g column eluting with ACN/Water 5/95 to 55/45 with acid modifier (0.1% TFA) to afford compound 250I (90 mg, 36%) after freeze-drying the product containing fractions. Analytical method 7; t_{R}=1.07 min; [M+H]⁺=1108.60.

Intermediate 91A in Table 7 below prepared according to the procedure used for Intermediate 250I (Example 2.10, intermediate). The intermediate 91A was obtained as byproduct in the synthesis of 250I.

TABLE 7

| Cmd | | Synthesis | |
|---|---|---|---|
| No. | Structure | Procedure | LCMS |
| 91A | 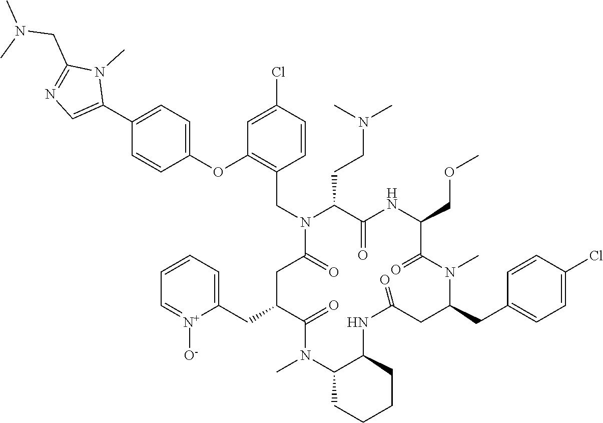 [SMILES: COC[C@@H]1NC(=O)[C@@H](CCN(C)C)N(Cc2ccc(Cl)cc2Oc2ccc(-c3cnc(CN(C)C)n3C)cc2)C(=O)C[C@@H](Cc2cccc[n+]2[O-])C(=O)N(C)[C@H]2CCCC[C@@H]2NC(=O)C[C@H](Cc2ccc(Cl)cc2)N(C)C1=O] | Intermediate 250I (Example 2.10, intermediate)- obtained as byproduct in the synthesis of 250I. | Analytical method 4 t_{R} = 1.40 min [(M + 1)/2]⁺ = 555.2 |

### Example 2.11: Method L—Synthesis of (4S,7S,10S,16aS,20aS)-4-(4-chlorobenzyl)-11-(2-(difluoromethoxy)-6-(4-(2-((dimethylamino)methyl)-1-methyl-1H-imidazol-5-yl)phenoxy)benzyl)-7-(methoxymethyl)-5,10,16-trimethylhexadecahydrobenzo[l][1,4,7,11,14]pentaazacyclooctadecine-2,6,9,12,15(3H)-pentaone (Compound 196)

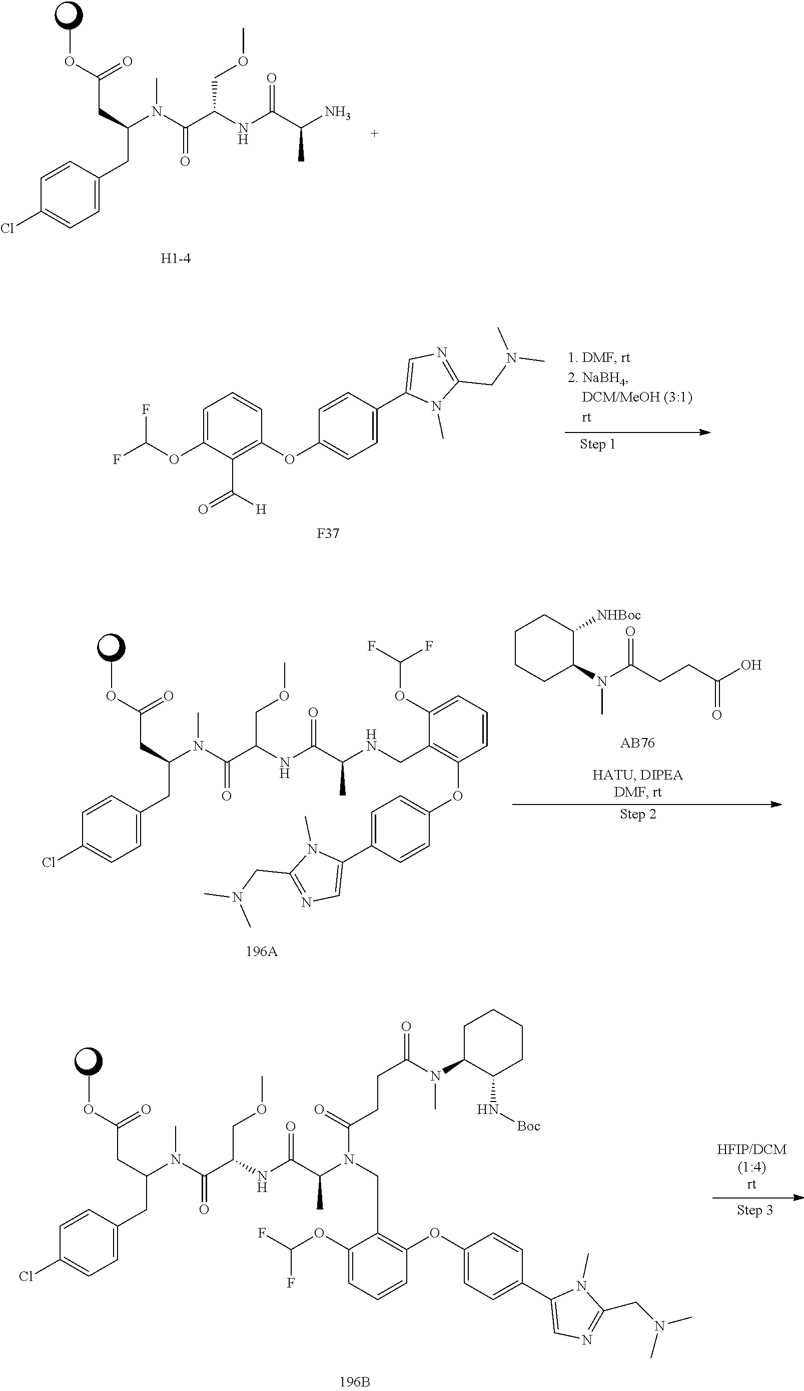 [SMILES: COC[C@H](NC(=O)[C@H](C)N)C(=O)N(C)[C@H](CC(=O)OC)Cc1ccc(Cl)cc1 ; CN(C)Cc1ncc(-c2ccc(Oc3cccc(OC(F)F)c3C=O)cc2)n1C ; COCC(NC(=O)[C@H](C)NCc1c(Oc2ccc(-c3cnc(CN(C)C)n3C)cc2)cccc1OC(F)F)C(=O)N(C)[C@H](CC(=O)OC)Cc1ccc(Cl)cc1 ; "F37" ; CN(C(=O)CCC(=O)O)[C@H]1CCCC[C@@H]1NC(=O)OC(C)(C)C ; COC[C@H](NC(=O)[C@H](C)N(Cc1c(Oc2ccc(-c3cnc(CN(C)C)n3C)cc2)cccc1OC(F)F)C(=O)CCC(=O)N(C)[C@H]1CCCC[C@@H]1NC(=O)OC(C)(C)C)C(=O)N(C)C(CC(=O)OC)Cc1ccc(Cl)cc1]

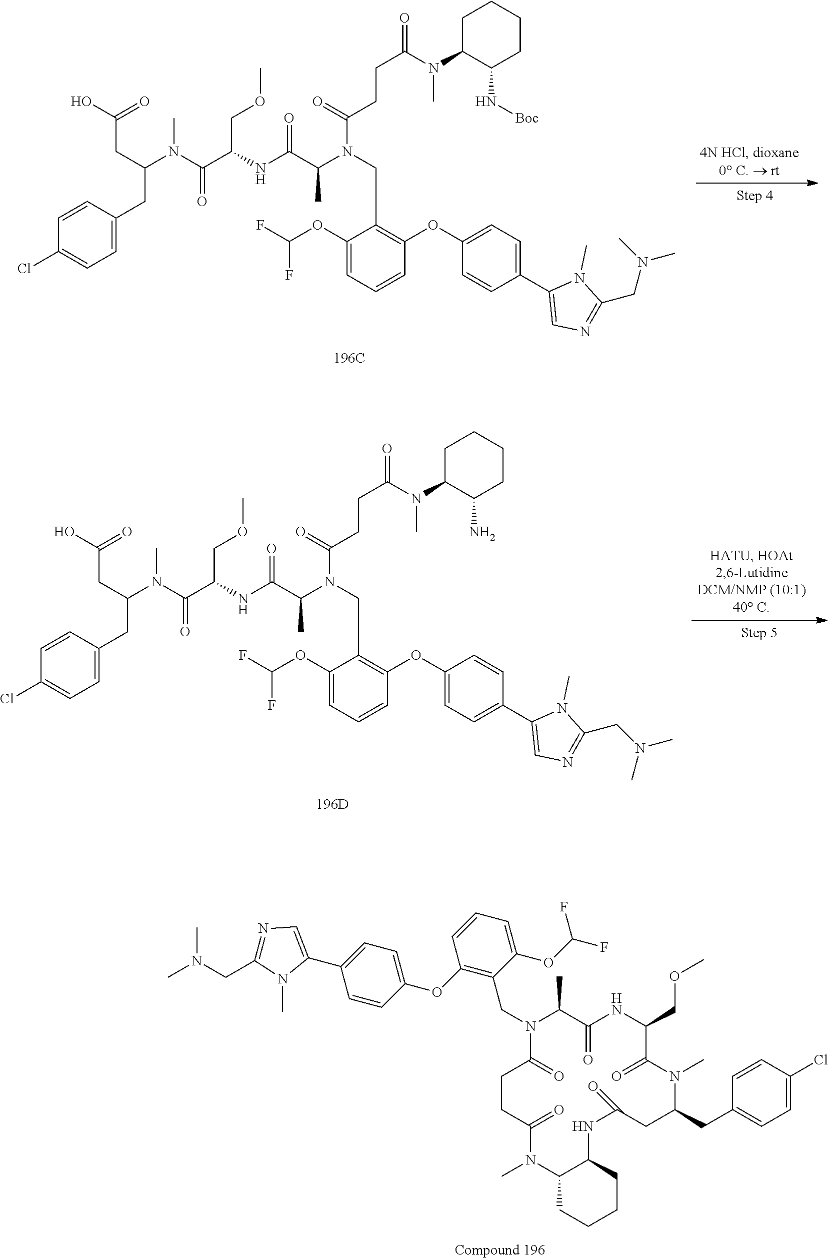

### Step 1. PS-2-chlorotrityl (S)-4-(4-chlorophenyl)-3-((S)-2-((S)-2-((2-(difluoromethoxy)-6-(4-(2-((dimethylamino)methyl)-1-methyl-1H-imidazol-5-yl)phenoxy)benzyl)amino)propanamido)-3-methoxy-N-methylpropanamido)butanoate (196A)

F37 (356 mg, 0.887 mmol) was added in one portion via glass pipette as a solution in DMF (2 mL with a 2 mL wash) to intermediate H1-4 (524 mg, 0.330 mmol) in a 15 mL polypropylene tube. The tube was capped and placed on a mechanical shaker at rt overnight. The resin was then filtered (with DCM, DMF, DCM, DMF, and DCM washing) and transferred to a 40 mL vial. 3:1 DCM:MeOH (6 mL) was added, followed by the addition of NaBH₄ (125 mg, 3.30 mmol) in one portion. The reaction was allowed to sit at rt over the weekend, after which it was filtered (with MeOH, DCM, DMF, DCM, DMF, DCM, DMF, and DCM washing) to provide 196A. Test cleavage of an aliquot (tip of a spatula) using 20% hexafluoroisopropanol:DCM showed a major peak corresponding to the desired cleaved product [M+H]+ by LCMS. The material was taken forward to the next step. Analytical method 5; t_{R}=0.72 min; [M+H]⁺=784.9.

### Step 2. PS-2-chlorotrityl (8S,11S,14S)-2-((1S,2S)-2-((tert-butoxycarbonyl)amino)cyclohexyl)-14-(4-chlorobenzyl)-7-(2-(difluoromethoxy)-6-(4-(2-((dimethylamino)methyl)-1-methyl-1H-imidazol-5-yl)phenoxy)benzyl)-11-(methoxymethyl)-8,13-dimethyl-3,6,9,12-tetraoxo-2,7,10,13-tetraazahexadecan-16-oate (196B)

AB76 (325 mg, 0.990 mmol) was added in one portion as a solution in DMF (3 mL with 2×1.5 mL washes) to intermediate 196A (0.330 mmol) in a 50 mL polypropylene tube. Next, DIPEA (0.35 mL, 1.980 mmol) was added in one portion via syringe, followed by the addition of HATU (376 mg, 0.990 mmol) in one portion. The tube was capped and placed on a mechanical shaker at rt overnight. The resin (196B) was filtered (with DCM, DMF, DCM, DMF, DCM, DMF, and DCM washing) and transferred to a 10 mL filter syringe in preparation for the next step.

### Step 3. (8S,11S,14S)-2-((1S,2S)-2-((tert-butoxycarbonyl)amino)cyclohexyl)-14-(4-chlorobenzyl)-7-(2-(difluoromethoxy)-6-(4-(2-((dimethylamino)methyl)-1-methyl-1H-imidazol-5-yl)phenoxy)benzyl)-11-(methoxymethyl)-8,13-dimethyl-3,6,9,12-tetraoxo-2,7,10,13-tetraazahexadecan-16-oic acid (196C)

Hexafluoroisopropanol/DCM (1:4) (6 mL) was taken up into a 10 mL filter syringe containing intermediate 196B (458 mg, 0.330 mmol). The syringe was placed on a mechanical shaker at rt for 20 mins, after which the filtrate was collected. This procedure was repeated with an additional 6 mL of 20% hexafluoroisopropanol:DCM (with shaking for 15 mins at rt). The resin was washed with DCM (×3), and the combined filtrate was concentrated in vacuo to provide a crude tan oil. The tan oil was taken up in MeOH:DMSO (1:1, 4.5 mL), filtered, and purified by reversed-phase basic HPLC (15-40% MeCN:H₂O (+5 mM NH₄OH), 1×1.5 mL injection on the 30×100 mm 5 m column (t_{R}=8.31 min), 2×1.5 mL injections on the 30×50 mm 5 m column (t_{R}=3.90 min)). The product-containing fractions were combined, partially concentrated in vacuo, frozen, and lyophilized to provide 196C (35 mg, 10%) as a white powder. Analytical method 4; t_{R}=2.14 min; [M+H]⁺=1094.8.

### Step 4. (8S,11S,14S)-2-((1S,2S)-2-aminocyclohexyl)-14-(4-chlorobenzyl)-7-(2-(difluoromethoxy)-6-(4-(2-((dimethylamino)methyl)-1-methyl-1H-imidazol-5-yl)phenoxy)benzyl)-11-(methoxymethyl)-8,13-dimethyl-3,6,9,12-tetraoxo-2,7,10,13-tetraazahexadecan-16-oic acid (196D)

4 N HCl in dioxane (0.5 mL, 2.00 mmol) was added in one portion via syringe to intermediate 196C (35 mg, 0.032 mmol) in dioxane (3 mL) in a round bottom flask charged with a magnetic stir bar at 0° C. The reaction was allowed to warm to rt and stir under N₂ for 4 hours, after which it was concentrated in vacuo to provide a light orange/tan residue. DCM was added, after which it was concentrated in vacuo again. This procedure was repeated ×2 to provide 196D (32 mg) as a light orange/tan solid, which was taken on to the next step without additional purification. Analytical method 7; t_{R}=0.80 min; [M+H]+=995.6.

### Step 5. (4S,7S,10S,16aS,20aS)-4-(4-chlorobenzyl)-11-(2-(difluoromethoxy)-6-(4-(2-((dimethylamino)methyl)-1-methyl-1H-imidazol-5-yl)phenoxy)benzyl)-7-(methoxymethyl)-5,10,16-trimethylhexadecahydro-benzo[l][1,4,7,11,14]pentaazacyclooctadecine-2,6,9,12,15(3H)-pentaone (Compound 196)

2,6-Lutidine (0.036 mL, 0.310 mmol) was added in one portion via syringe to a suspension of 196D (32 mg, 0.031 mmol) in DCM (31 mL) in a round bottom flask charged with a magnetic stir bar at rt. Next, HATU (24 mg, 0.062 mmol) was added in one portion, followed by the addition of HOAt (8.4 mg, 0.062 mmol) in one portion. Finally, NMP (3.1 mL) was added in one portion via syringe, and the reaction was heated to 40° C. overnight. Following overnight reaction, the mixture was partially concentrated in vacuo, after which it was diluted with EtOAc (100 mL), washed with H₂O (2×75 mL) and brine (50 mL), dried over Na₂SO₄, filtered, and concentrated in vacuo to provide a crude orange oil. The oil was taken up in MeOH:DMSO (1:1, 3 mL), filtered, and purified via reverse phase basic HPLC (35-60% MeCN:H₂O (+5 mM NH₄OH), 2×1.5 mL injections, 30×100 mm 5 m column, t_{R}=6.84 min). The main product-containing fractions were combined, partially concentrated in vacuo, frozen, and lyophilized to provide Compound 196 (6.7 mg, 22%) as a white powder. Analytical method 4; t_{R}=1.92 min; [M+H]+=976.9.

### Example 3: Post Modifications

In this section, the modifications that were done after cyclization of the linear peptides are described herein.

### Example 3.1: Synthesis of (2S,5S,8S,13S,16R)-16-benzyl-1-(4-chloro-2-(4-(5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone trifluoroacetate (Compound 121) and (2S,5S,8S,13S,16R)-16-benzyl-1-(4-chloro-2-(4-(7-(pyrrolidine-3-carbonyl)-5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone trifluoroacetate (Compound 124)

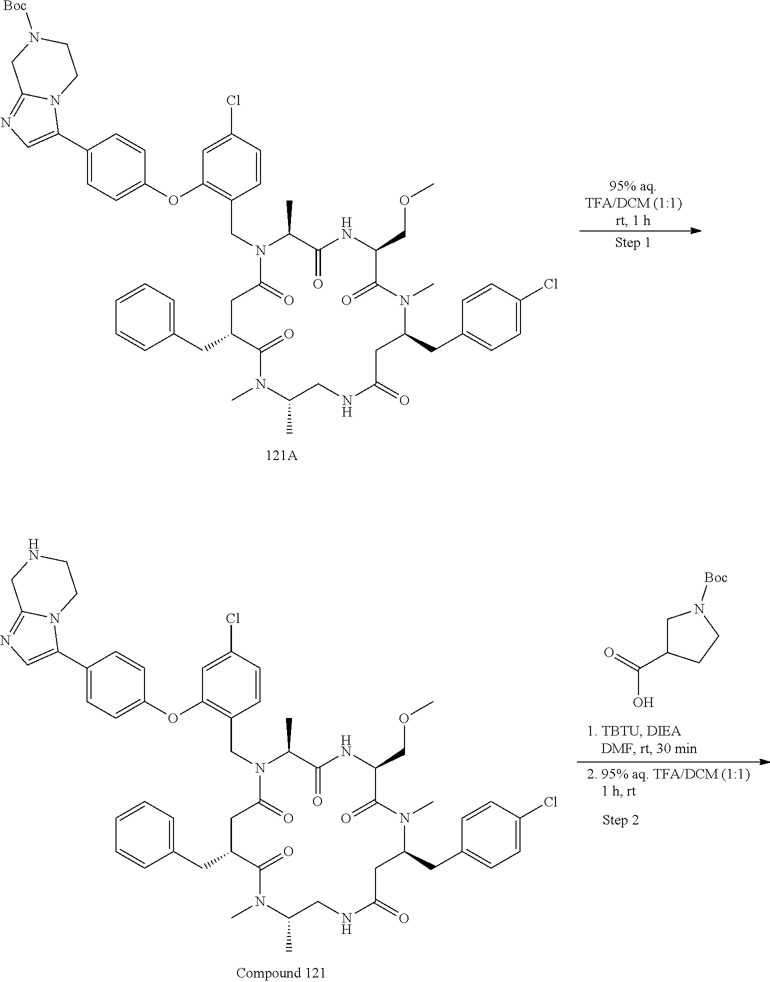 [SMILES: COC[C@@H]1NC(=O)[C@H](C)N(Cc2ccc(Cl)cc2Oc2ccc(-c3cnc4n3CCN(C(=O)OC(C)(C)C)C4)cc2)C(=O)C[C@@H](Cc2ccccc2)C(=O)N(C)[C@@H](C)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cnc4n3CCNC4)cc2)C(=O)C[C@@H](Cc2ccccc2)C(=O)N(C)[C@@H](C)CNC(=O)C[C@H](Cc2ccc(Cl)cc2)N(C)C1=O ; CC(C)(C)OC(=O)N1CCC(C(=O)O)C1]

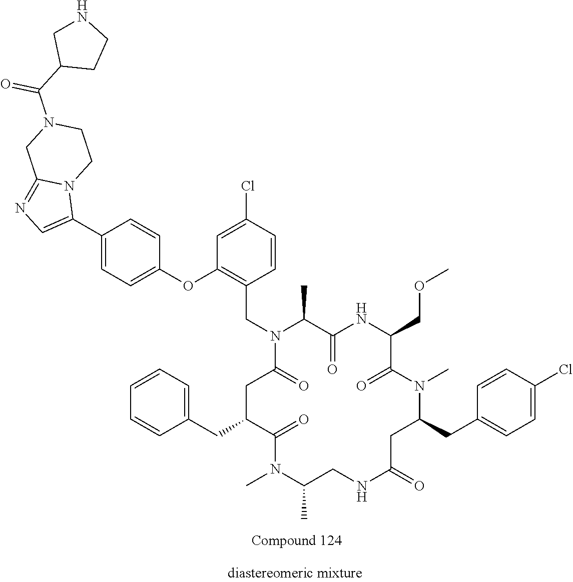 [SMILES: COC[C@@H]1NC(=O)[C@H](C)N(Cc2ccc(Cl)cc2Oc2ccc(-c3cnc4n3CCN(C(=O)C3CCNC3)C4)cc2)C(=O)C[C@@H](Cc2ccccc2)C(=O)N(C)[C@@H](C)CNC(=O)C[C@H](Cc2ccc(Cl)cc2)N(C)C1=O]

### Step 1. (2S,5S,8S,13S,16R)-16-benzyl-1-(4-chloro-2-(4-(5,6,7,8-tetrahydroimidazo[1,2-a]pyrazin-3-yl)-phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone trifluoroacetate (Compound 121)

Intermediate 121A (0.120 mmol) was dissolved in 95% aq. TFA/DCM (1:1) (10 mL) and the resulting mixture was stirred for 1 h at rt and then concentrated to dryness in vacuo. The crude product was purified by preparative reversed-phase HPLC (eluent A: 0.1% TFA in H₂O and eluent B: ACN). Pure fractions were combined and lyophilized to afford Compound 121 (56 mg, 0.046 mmol, 38% yield) as a white solid. Analytical method 12; t_{R}=4.31 min; [M+H]⁺=979.5, [M+2H]²⁺=490.3, [M−H]⁻=977.5.

### Step 2. (2S,5S,8S,13S,16R)-16-benzyl-1-(4-chloro-2-(4-(7-(pyrrolidine-3-carbonyl)-5,6,7,8-tetrahydroimidazo-[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone trifluoroacetate (Compound 124)

N-Boc-pyrrolidine-3-carboxylic acid (5.48 mg, 0.025 mmol) and TBTU (8.17 mg, 0.025 mmol) were dissolved in DMF (0.5 mL) and DIEA (4.45 μL, 0.025 mmol). The solution was stirred for 2 min at rt, and then a solution of Compound 121 (20.5 mg, 0.017 mmol) and DIEA (8.89 μL, 0.051 mmol) in DMF (1.5 mL) was added. The reaction mixture was stirred for 30 min at rt, and then partitioned between EtOAc (30 mL) and 5% aq. NaHCO₃ (5 mL). The organic layer was washed with 5% aq. NaHCO₃ (3×5 mL) and brine (5 mL), dried over Na₂SO₄, filtered, and concentrated to dryness in vacuo. The crude product was dissolved in 95% aq. TFA/DCM (1:1) (5 mL) and the resulting mixture stirred for 1 h at rt and then concentrated to dryness in vacuo. The crude product was purified by preparative HPLC (eluent A: 0.1% TFA in H₂O and eluent B: ACN). Pure fractions were combined and lyophilized to afford Compound 124 (diastereomeric mixture) (19.5 mg, 0.015 mmol, 86% yield) as a white solid. Analytical method 9; t_{R}=4.02 min; [M+H]⁺=1076.5, [M+2H]²⁺=538.7.

The Compounds shown in Table 8 below were prepared according to the procedure used for Compound 124 (Example 3.1, Steps 1 & 2) or Compound 121 (Example 3.1, Step 1).

TABLE 8

| Cmd | | Synthesis | |
|---|---|---|---|
| No. | Structure | Procedure | LCMS |
| 114 | 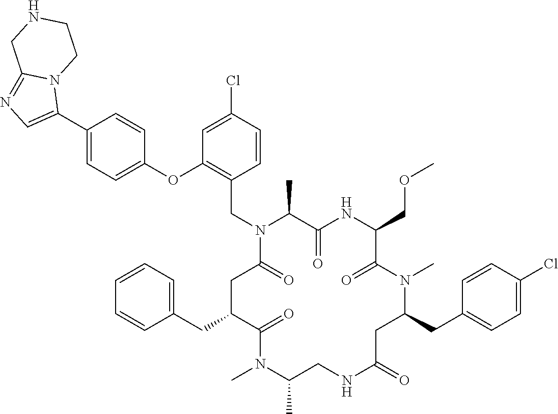 [SMILES: COC[C@@H]1NC(=O)[C@H](C)N(Cc2ccc(Cl)cc2Oc2ccc(-c3cnc4n3CCNC4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1) | Analytical method 15 t_{R} = 2.22 min [M + H]⁺ = 980.4 |
| 115 | 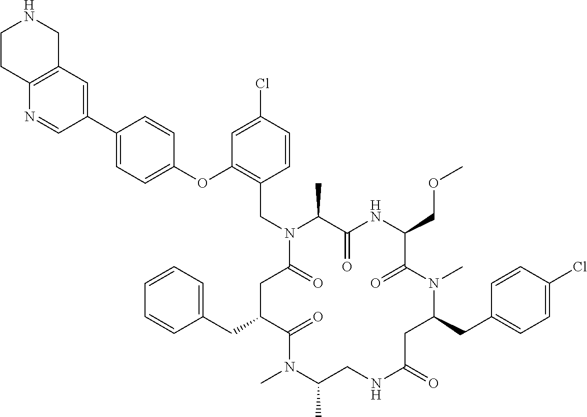 [SMILES: COC[C@@H]1NC(=O)[C@H](C)N(Cc2ccc(Cl)cc2Oc2ccc(-c3cnc4c(c3)CNCC4)cc2)C(=O)C[C@@H](Cc2ccccc2)C(=O)N(C)[C@@H](C)CNC(=O)C[C@H](Cc2ccc(Cl)cc2)N(C)C1=O] | Compound 121 (Example 3.1, Step 1)- starting from intermediate 115A. | Analytical method 15 t_{R} = 2.30 min [M + H]⁺ = 990.4 |
| 116 | 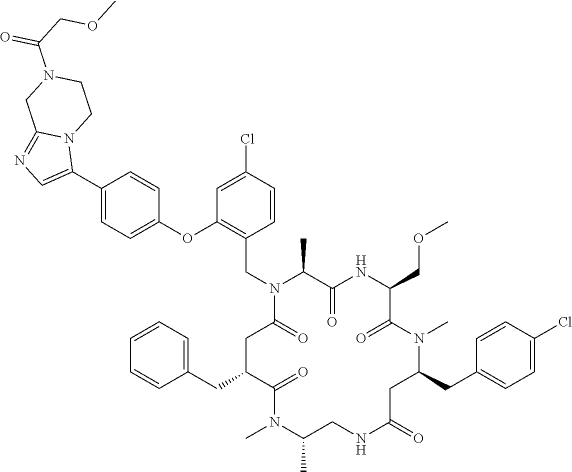 [SMILES: COCC(=O)N1CCn2c(-c3ccc(Oc4cc(Cl)ccc4CN4C(=O)C[C@@H](Cc5ccccc5)C(=O)N(C)[C@@H](C)CNC(=O)C[C@H](Cc5ccc(Cl)cc5)N(C)C(=O)[C@H](COC)NC(=O)[C@@H]4C)cc3)cnc2C1] | Compound 124 (Example 3.1)- starting from Compound 114 and 2-methoxyacetic acid | Analytical method 15 t_{R} = 2.51 min [M + H]⁺ = 1052.4 |
| 117 | 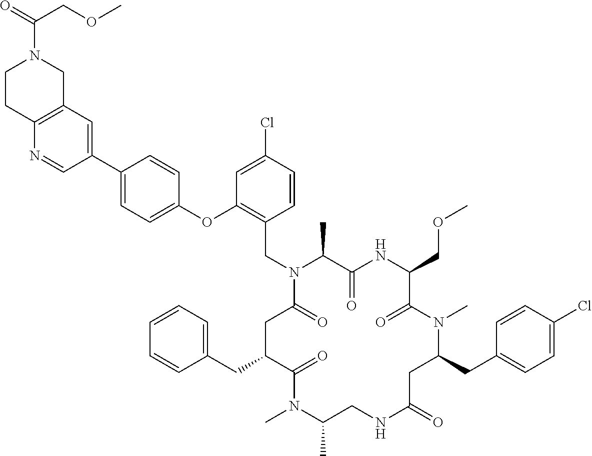 [SMILES: COCC(=O)N1CCc2ncc(-c3ccc(Oc4cc(Cl)ccc4CN4C(=O)C[C@@H](Cc5ccccc5)C(=O)N(C)[C@@H](C)CNC(=O)C[C@H](Cc5ccc(Cl)cc5)N(C)C(=O)[C@H](COC)NC(=O)[C@@H]4C)cc3)cc2C1] | Compound 124 (Example 3.1)- starting from Compound 115 and 2-methoxyacetic acid. | Analytical method 15 t_{R} = 2.46 min [M + H]⁺ = 1062.4 |
| 119 | 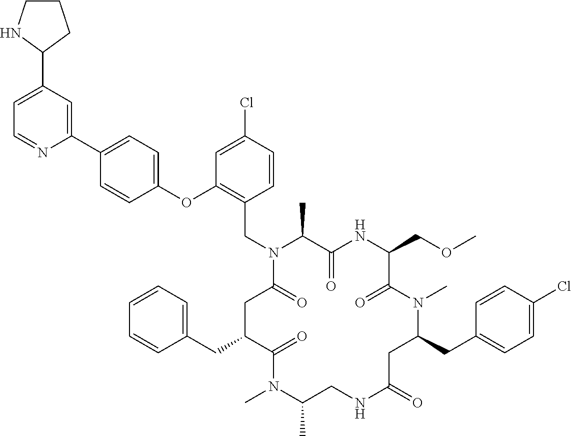 [SMILES: COC[C@@H]1NC(=O)[C@H](C)N(Cc2ccc(Cl)cc2Oc2ccc(-c3cc(C4CCCN4)ccn3)cc2)C(=O)C[C@@H](Cc2ccccc2)C(=O)N(C)[C@@H](C)CNC(=O)C[C@H](Cc2ccc(Cl)cc2)N(C)C1=O] | Compound 121 (Example 3.1, Step 1)- starting from intermediate 119A. | Analytical method 10 t_{R} = 1.08 min [M + H]⁺ = 1005.0 |
| 125 | 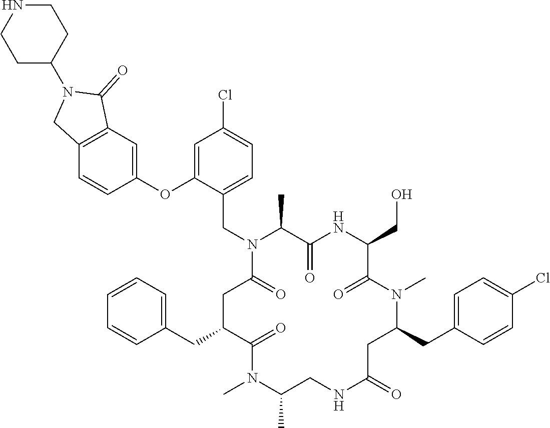 [SMILES: C[C@H]1CNC(=O)C[C@H](Cc2ccc(Cl)cc2)N(C)C(=O)[C@H](CO)NC(=O)[C@H](C)N(Cc2ccc(Cl)cc2Oc2ccc3c(c2)C(=O)N(C2CCNCC2)C3)C(=O)C[C@@H](Cc2ccccc2)C(=O)N1C] | Compound 121 (Example 3.1, Step 1)- starting from intermediate 125A. | Analytical method 4 t_{R} = 1.91 min [M + H]⁺ = 984.2 |
| 129 | 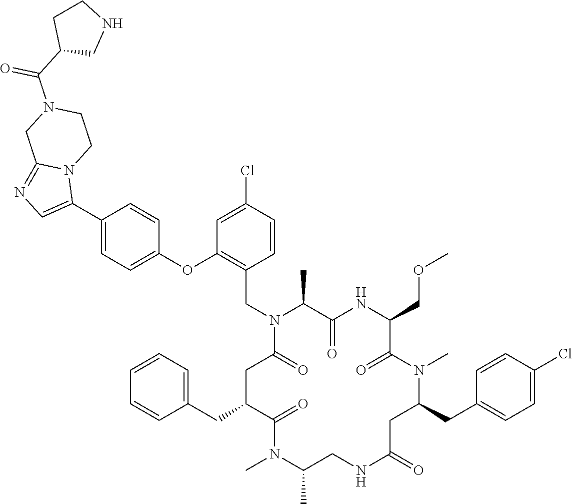 [SMILES: COC[C@@H]1NC(=O)[C@H](C)N(Cc2ccc(Cl)cc2Oc2ccc(-c3cnc4n3CCN(C(=O)[C@@H]3CCNC3)C4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1)- starting from Compound 114 and (R)-1-(tert- butoxycarbonyl)- pyrrolidine-3- carboxylic acid. | Analytical method 4 t_{R} = 2.01 min [M + 2H]²⁺ = 540.5 |
| 131 | 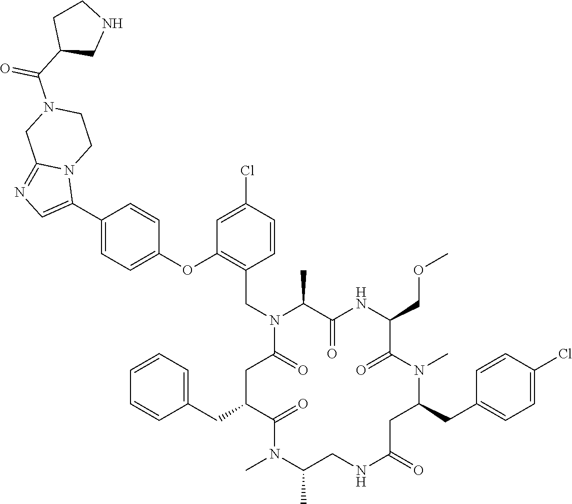 [SMILES: COC[C@@H]1NC(=O)[C@H](C)N(Cc2ccc(Cl)cc2Oc2ccc(-c3cnc4n3CCN(C(=O)[C@H]3CCNC3)C4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1)- starting from Compound 114 and (S)-1-(tert- butoxycarbonyl)- pyrrolidine-3- carboxylic acid. | Analytical method 4 t_{R} = 1.99 min [M + 2H]²⁺ = 540.5 |
| 132 | 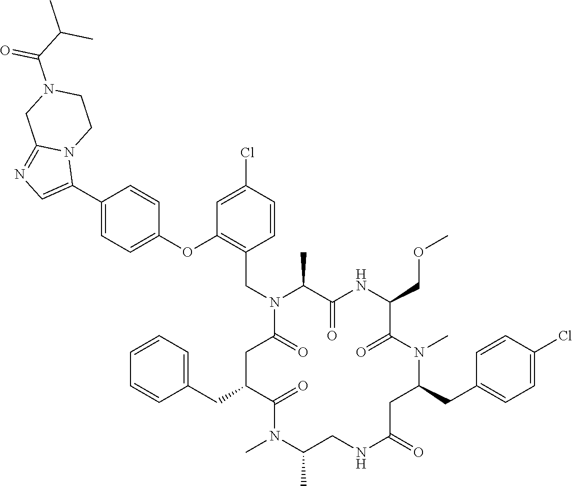 [SMILES: COC[C@@H]1NC(=O)[C@H](C)N(Cc2ccc(Cl)cc2Oc2ccc(-c3cnc4n3CCN(C(=O)C(C)C)C4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1)- startmg from Compound 121 and isobutyric acid. | Analytical method 13 t_{R} = 4.60 min [M + H]⁺ = 1049.5 |
| 133 | 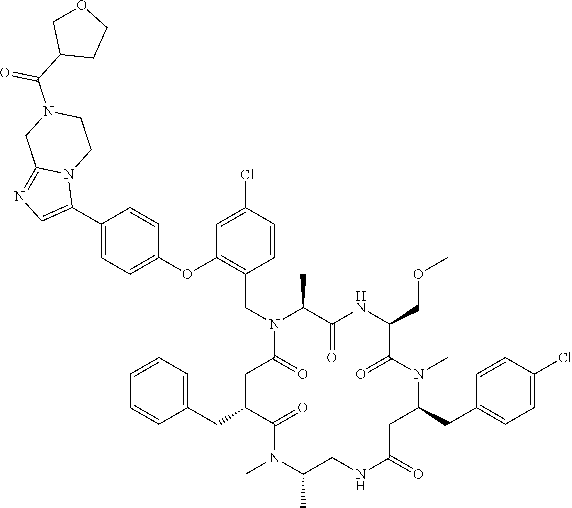 [SMILES: COC[C@@H]1NC(=O)[C@H](C)N(Cc2ccc(Cl)cc2Oc2ccc(-c3cnc4n3CCN(C(=O)C3CCOC3)C4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1)- starting from Compound 121 and tetrahydrofuran-3- carboxylic acid. | Analytical method 13 t_{R} = 4.41 min [M + H]⁺ = 1077.5 |
| 136 | 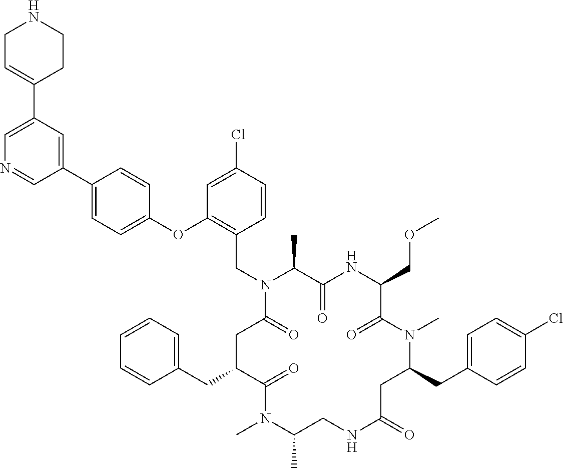 [SMILES: COC[C@@H]1NC(=O)[C@H](C)N(Cc2ccc(Cl)cc2Oc2ccc(-c3cncc(C4=CCNCC4)c3)cc2)C(=O)C[C@@H](Cc2ccccc2)C(=O)N(C)[C@@H](C)CNC(=O)C[C@H](Cc2ccc(Cl)cc2)N(C)C1=O] | Compound 121 (Example 3.1, Step 1)- starting from intermediate 136A. | Analytical method 13 t_{R} = 4.07 min [M + H]⁺ = 1016.4 |
| 137 | 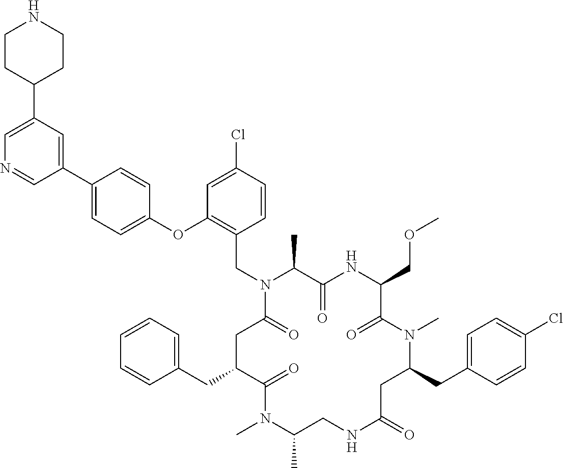 [SMILES: COC[C@@H]1NC(=O)[C@H](C)N(Cc2ccc(Cl)cc2Oc2ccc(-c3cncc(C4CCNCC4)c3)cc2)C(=O)C[C@@H](Cc2ccccc2)C(=O)N(C)[C@@H](C)CNC(=O)C[C@H](Cc2ccc(Cl)cc2)N(C)C1=O] | Compound 121 (Example 3.1, Step 1)- starting from intermediate 137A. | Analytical method 13 t_{R} = 3.92 min [M + H]⁺ = 1018.4 |
| 142 | 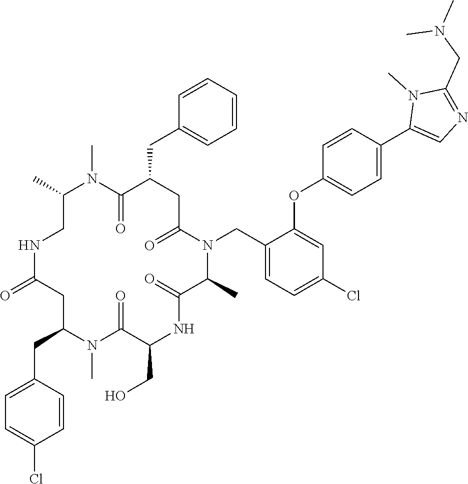 [SMILES: C[C@H]1CNC(=O)C[C@H](Cc2ccc(Cl)cc2)N(C)C(=O)[C@H](CO)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Compound 121 (Example 3.1, Step 1)- starting from 142A. | Analytical method 2 t_{R} = 2.66 min [M + H]⁺ = 981.5 |
| 146 | 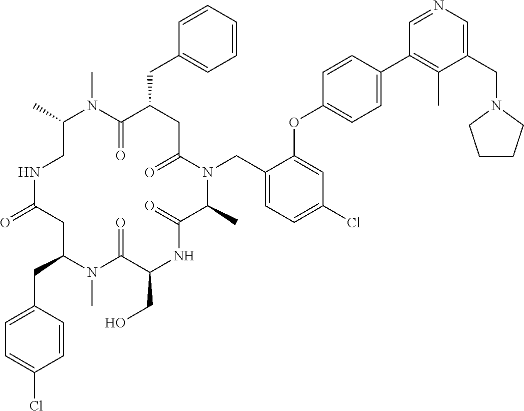 [SMILES: Cc1c(CN2CCCC2)cncc1-c1ccc(Oc2cc(Cl)ccc2CN2C(=O)C[C@@H](Cc3ccccc3)C(=O)N(C)[C@@H](C)CNC(=O)C[C@H](Cc3ccc(Cl)cc3)N(C)C(=O)[C@H](CO)NC(=O)[C@@H]2C)cc1] | Compound 121 (Example 3.1, Step 1)- starting from 146A. | Analytical method 2 t_{R} = 3.16 min [M + H]⁺ = 1018.4 |
| 162 | 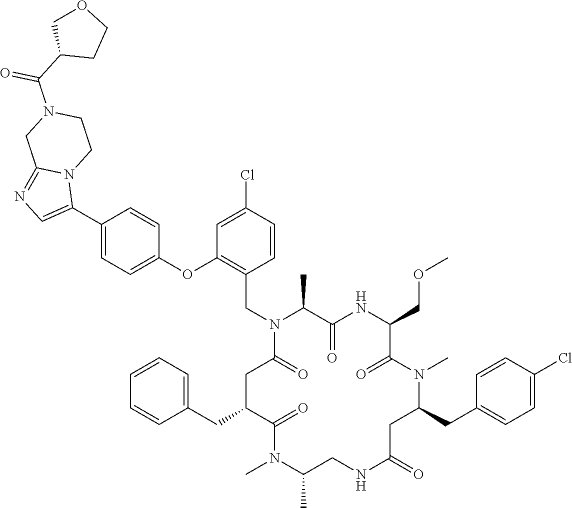 [SMILES: COC[C@@H]1NC(=O)[C@H](C)N(Cc2ccc(Cl)cc2Oc2ccc(-c3cnc4n3CCN(C(=O)[C@H]3CCOC3)C4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1)- starting from Compound 121 and (S)- tetrahydrofuran- 3-carboxylic acid. | Analytical method 13 t_{R} = 4.38 min [M + H]⁺ = 1077.4 |
| 171 | 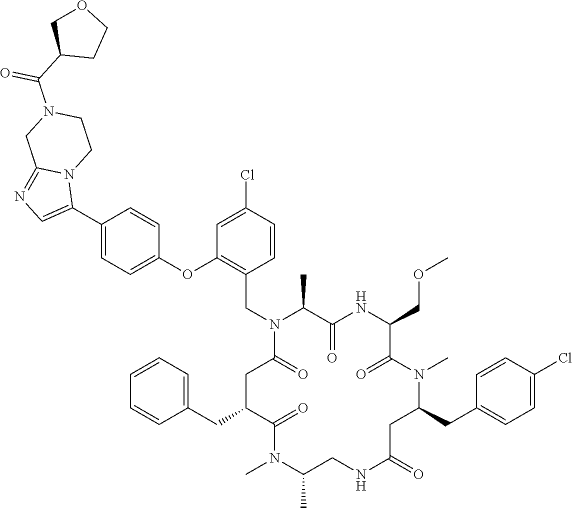 [SMILES: COC[C@@H]1NC(=O)[C@H](C)N(Cc2ccc(Cl)cc2Oc2ccc(-c3cnc4n3CCN(C(=O)[C@@H]3CCOC3)C4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1)- starting from Compound 121 and (R)- tetrahydrofuran- 3-carboxylic acid. | Analytical method 13 t_{R} = 4.38 min [M + H]⁺ = 1077.4 |
| 176 | 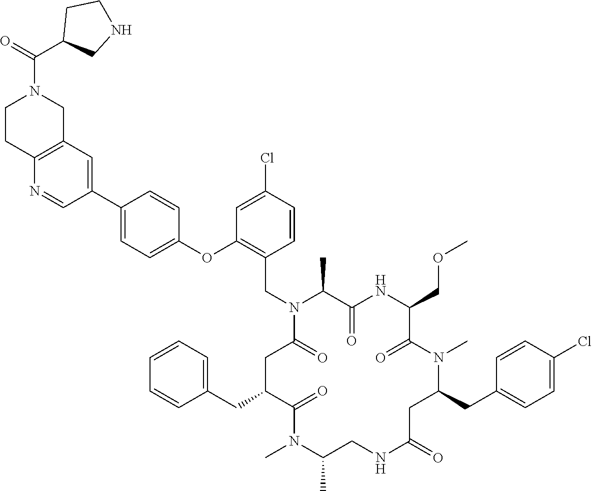 [SMILES: COC[C@@H]1NC(=O)[C@H](C)N(Cc2ccc(Cl)cc2Oc2ccc(-c3cnc4c(c3)CN(C(=O)[C@H]3CCNC3)CC4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1)- starting from Compound 115 and (S)-1-(tert- butoxycarbonyl)- pyrrolidine- 3-carboxylic acid. | Analytical method 13 t_{R} = 4.02 min [M + H]⁺ = 1087.5 |
| 212 | 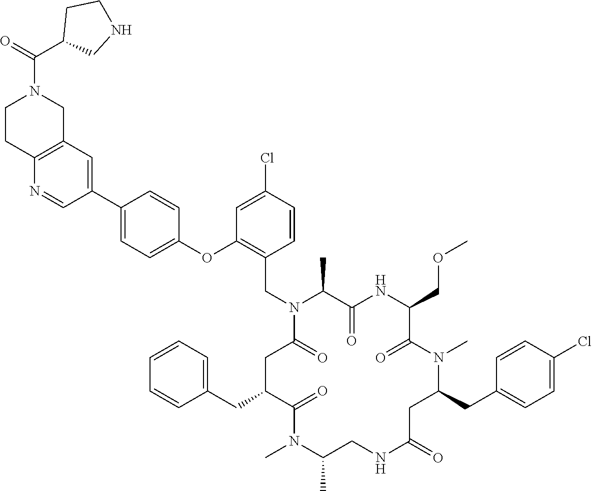 [SMILES: COC[C@@H]1NC(=O)[C@H](C)N(Cc2ccc(Cl)cc2Oc2ccc(-c3cnc4c(c3)CN(C(=O)[C@@H]3CCNC3)CC4)cc2)C(=O)C[C@@H](Cc2ccccc2)C(=O)N(C)[C@@H](C)CNC(=O)C[C@H](Cc2ccc(Cl)cc2)N(C)C1=O] | Compound 124 (Example 3.1)- starting from Compound 115 and (R)-1-(tert- butoxycarbonyl)- pyrrolidine- 3-carboxylic acid. | Analytical method 13 t_{R} = 4.02 min [M + H]⁺ = 1087.5 |
| 220 | 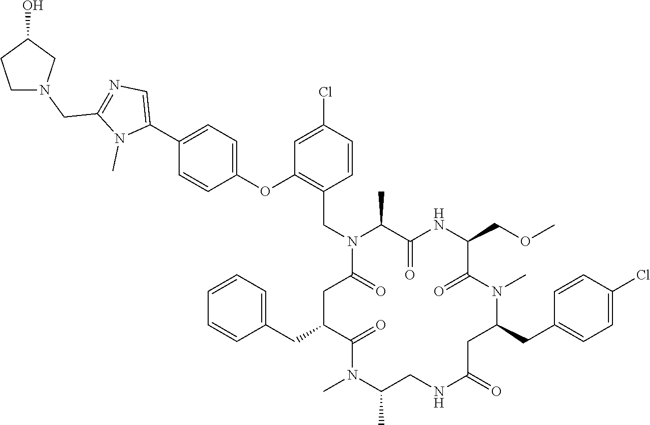 [SMILES: COC[C@@H]1NC(=O)[C@H](C)N(Cc2ccc(Cl)cc2Oc2ccc(-c3cnc(CN4CC[C@H](O)C4)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 121 (Example 3.1, Step 1)- starting with TBS removal of Intermediate 220A according to Step 1 above. | Analytical method13 t_{R} = 4.20 min [M + H]⁺ = 1037.4 |

### Example 3.2: Synthesis of (2S,5S,8S,13S,16R)-16-benzyl-1-(4-chloro-2-(4-(7-isopropyl-5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone trifluoroacetate (Compound 123)

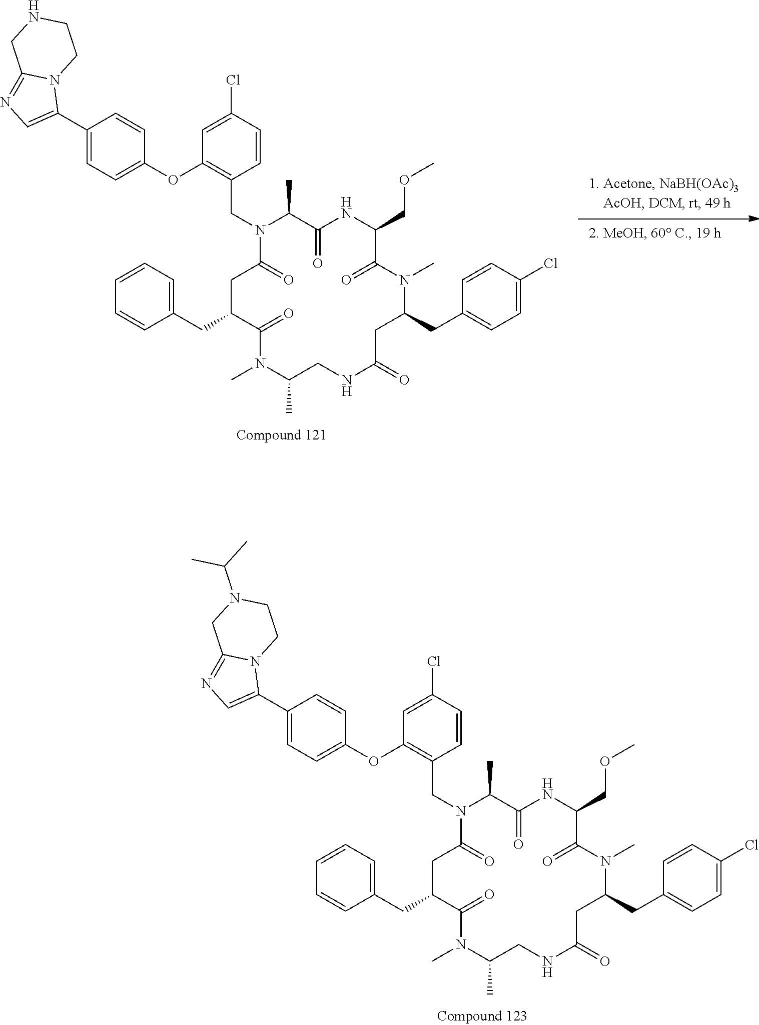 [SMILES: COC[C@@H]1NC(=O)[C@H](C)N(Cc2ccc(Cl)cc2Oc2ccc(-c3cnc4n3CCNC4)cc2)C(=O)C[C@@H](Cc2ccccc2)C(=O)N(C)[C@@H](C)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cnc4n3CCN(C(C)C)C4)cc2)C(=O)C[C@@H](Cc2ccccc2)C(=O)N(C)[C@@H](C)CNC(=O)C[C@H](Cc2ccc(Cl)cc2)N(C)C1=O]

To Compound 121 (18.1 mg, 15.0 μmol) dissolved in DCM (2 mL) was added acetone (3.3 μL, 45.0 μmol), AcOH (2.6 μL, 45.0 μmol), and NaBH(OAc)₃ (9.54 mg, 45.0 μmol) were added and the resulting mixture was stirred for 2 h 20 min at rt. More acetone (3.3 μL, 45.0 μmol) was then added and stirring was continued for 1 h 20 min. An additional amount of acetone (3.3 μL, 45.0 μmol) and NaBH(OAc)₃ (9.54 mg, 45.0 μmol) were added and stirring was continued for 45 h 20 min. MeOH (2 mL) was then added and the resulting solution was stirred for 19 h at 60° C. and then concentrated to dryness in vacuo. The crude product was purified by preparative reversed-phase HPLC (eluent A: 0.1% TFA in H₂O and eluent B: ACN). Pure fractions were combined and lyophilized to afford Compound 123 (12.8 mg, 9.52 μmol, 63.5% yield) as a light yellow solid. Analytical method 12; t_{R}=4.87 min; [M+H]⁺=1021.7, [M+2H]²⁺=511.5, [M−H]⁻=1019.8.

### Example 3.3: Synthesis of (2S,5S,8S,13S,16R)-16-benzyl-1-(4-chloro-2-((2-(1-methylpiperidin-4-yl)-3-oxoisoindolin-5-yl)oxy)benzyl)-8-(4-chlorobenzyl)-5-(hydroxymethyl)-2,7,13,14-tetramethyl-1,4,7,11,14-pentaazacyclooctadecane-3,6,10,15,18-pentaone (Compound 130)

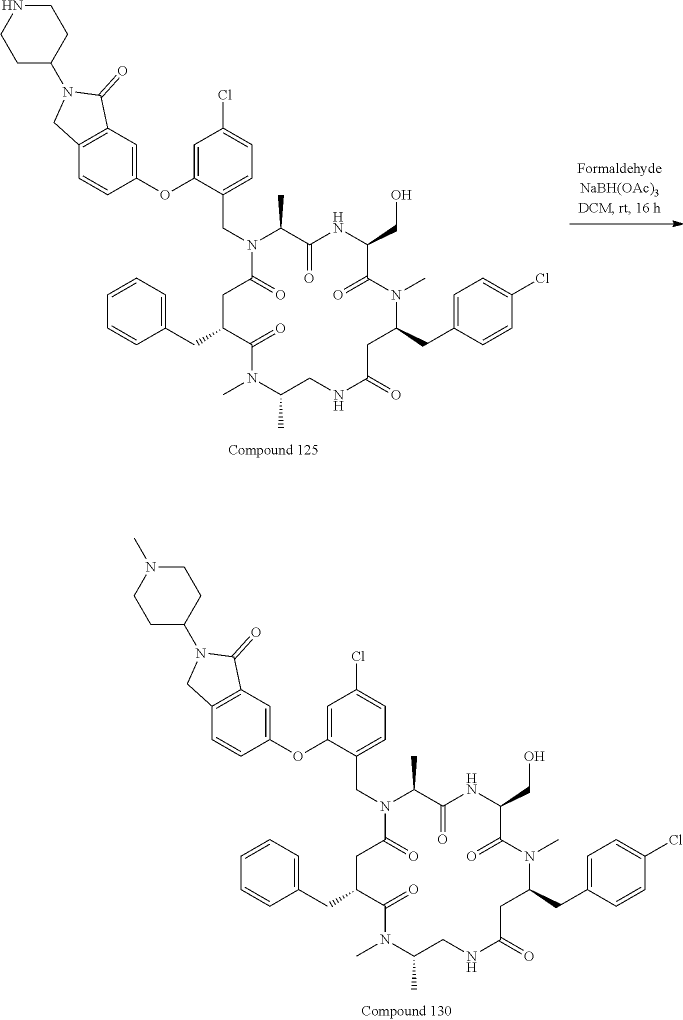 [SMILES: C[C@H]1CNC(=O)C[C@H](Cc2ccc(Cl)cc2)N(C)C(=O)[C@H](CO)NC(=O)[C@H](C)N(Cc2ccc(Cl)cc2Oc2ccc3c(c2)C(=O)N(C2CCNCC2)C3)C(=O)C[C@@H](Cc2ccccc2)C(=O)N1C ; C[C@H]1CNC(=O)C[C@H](Cc2ccc(Cl)cc2)N(C)C(=O)[C@H](CO)NC(=O)[C@H](C)N(Cc2ccc(Cl)cc2Oc2ccc3c(c2)C(=O)N(C2CCN(C)CC2)C3)C(=O)C[C@@H](Cc2ccccc2)C(=O)N1C]

To a solution of Compound 125 (10.3 mg, 10.48 μmol) in DCM (1 mL) was added formaldehyde (2.34 μL, 0.031 mmol) and NaBH(OAc)₃ (6.6 mg, 0.031 mmol) and the resulting mixture was stirred overnight at rt and then diluted with water. The pH of the reaction mixture was adjusted to pH 10 by the addition of Na₂CO₃ (sat) and the resulting mixture was extracted with DCM (2×). The combined organic layers were dried over Na₂SO₄, filtered, and concentrated under reduced pressure. The crude product was purified by reversed-phase chromatography on a C18 column eluting with 10-100% CH₃CN/H₂O with 0.01% TFA. The TFA salt of the product was salt exchanged using work up methods described above in the general preparative HPLC purification procedure and mass spectra section above to give Compound 130 (4.9 mg, 44.6%). Analytical method 4; t_{R}=1.89 min; [M+H]⁺=997.7.

TABLE 9
Compound 139.

| Cmd | | Synthesis | |
|---|---|---|---|
| No. | Structure | procedure | LCMS |
| 139 | 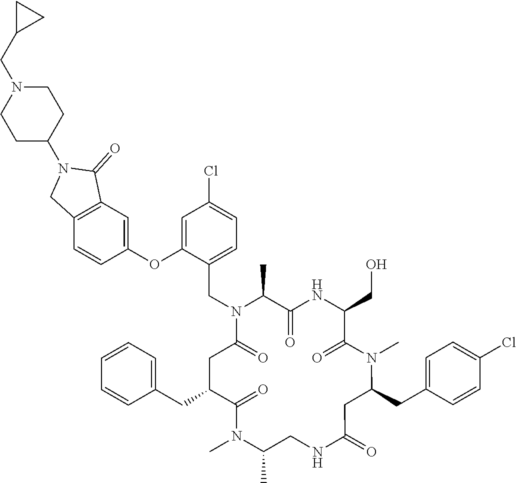 [SMILES: C[C@H]1CNC(=O)C[C@H](Cc2ccc(Cl)cc2)N(C)C(=O)[C@H](CO)NC(=O)[C@H](C)N(Cc2ccc(Cl)cc2Oc2ccc3c(c2)C(=O)N(C2CCN(CC4CC4)CC2)C3)C(=O)C[C@@H](Cc2ccccc2)C(=O)N1C] | Prepared according to the procedure used for Compound 130 (Example 3.3) | Analytical method 4 t_{R} = 1.96 min [M + H]⁺ = 1038.3 |

### Example 3.4: Synthesis of (2S,5S,8S,13S,16R)-16-benzyl-1-(4-chloro-2-(4-(5-(1-(2-fluoroethyl)piperidin-4-yl)pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone hydrochloride (Compound 84)

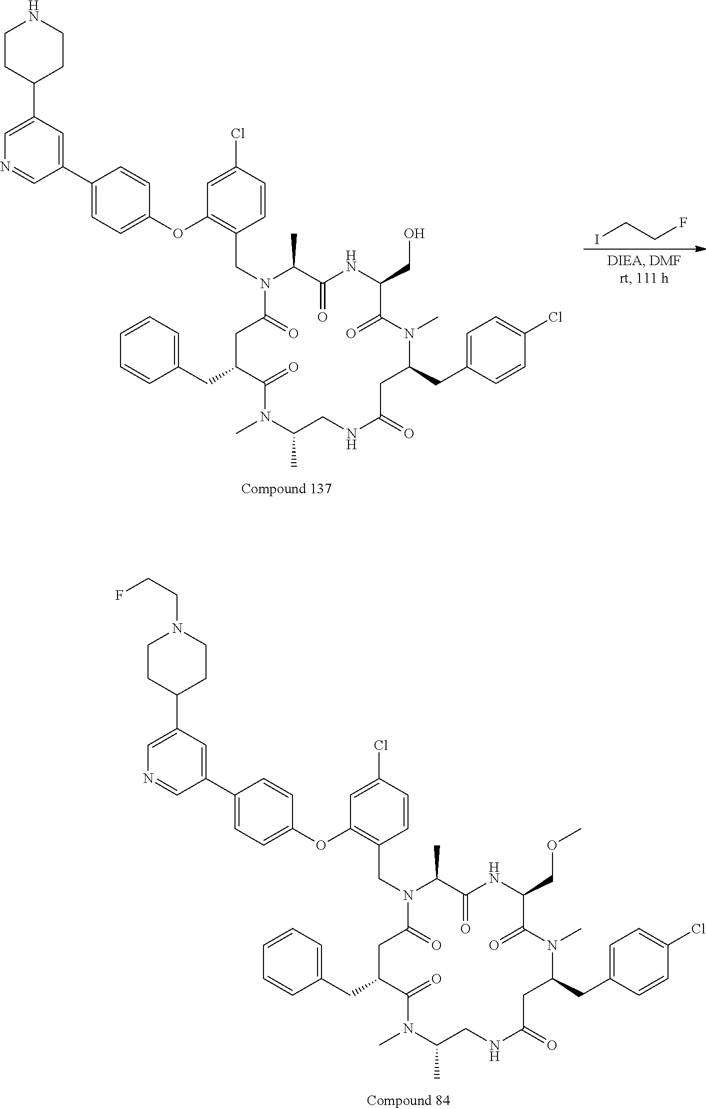

Compound 137 (37.5 mg, 0.030 mmol) was converted to the hydrochloride salt by lyophilization from a mixture of ACN/H₂O/HCl. The crude product was dissolved in DMF (10 mL) and 1-fluoro-2-iodoethane (7.3 μL, 0.090 mmol) und DIEA (0.047 mL, 0.271 mmol) were added. The reaction mixture was stirred for 16.5 h at 40° C. Additional 1-fluoro-2-iodoethane (2.4 μL, 0.030 mmol) was added and stirring was continued for 22 h. Additional 1-fluoro-2-iodoethane (2.4 μL, 0.030 mmol) was added and stirring was continued for 72.5 h. The crude product was purified by preparative HPLC (eluent A: 0.1% TFA in H₂O and eluent B: ACN). Pure fractions were combined and lyophilized to yield the product as a trifluoroacetate salt. The product was converted to the HCl salt via the free base (see general preparative HPLC purification procedure and mass spectra section above) to yield Compound 84 (17.2 mg, 15 μmol) as a white solid. Analytical method 12; t_{R}=4.42 min; [M+H]⁺=1064.6, [M−H]⁻=1062.5.

### Example 3.5: Synthesis of (2S,5S,8S,13S,16R)-13-(4-aminobutyl)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,14-trimethyl-1,4,7,11,14-pentaazacyclooctadecane-3,6,10,15,18-pentaone trifluoroacetate (Compound 169) and (2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-13-(4-(dimethylamino)butyl)-5-(methoxymethyl)-2,7,14-trimethyl-1,4,7,11,14-pentaazacyclooctadecane-3,6,10,15,18-pentaone hydrochloride (Compound 43)

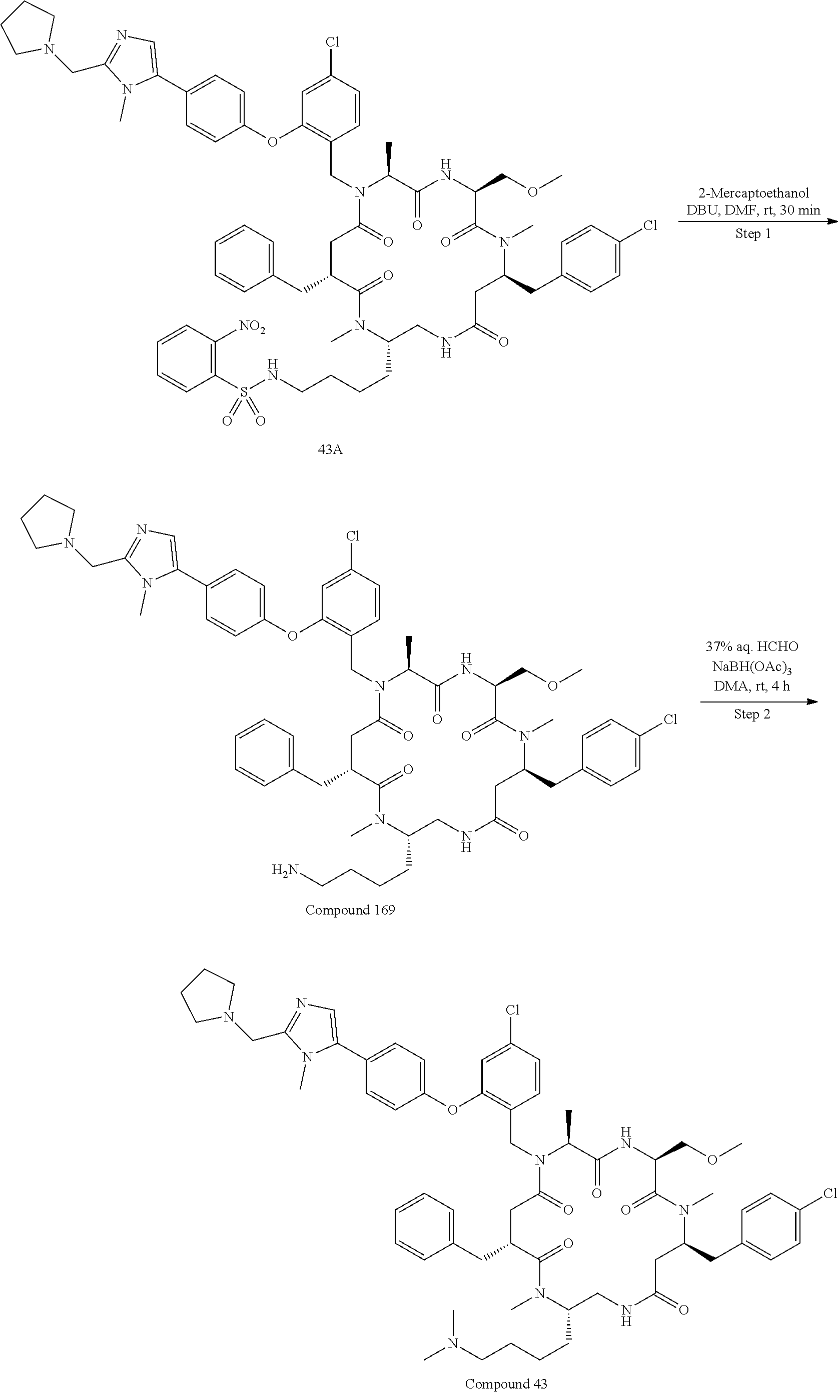 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](CCCCNS(=O)(=O)c2ccccc2[N+](=O)[O-])CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](CCCCN)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](CCCCN(C)C)CNC(=O)C[C@H](Cc2ccc(Cl)cc2)N(C)C1=O]

### Step 1. (2S,5S,8S,13S,16R)-13-(4-aminobutyl)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,14-trimethyl-1,4,7,11,14-pentaazacyclooctadecane-3,6,10,15,18-pentaone trifluoroacetate (Compound 169)

To a solution of intermediate 43A (532 mg, 0.271 mmol, 64.5%) in DMF (15 mL) was added 2-mercaptoethanol (0.115 mL, 1.628 mmol) and DBU (0.082 mL, 0.543 mmol) and the resulting mixture was stirred for 30 min at rt, then quenched by the addition of AcOH (0.4 mL), and concentrated to dryness in vacuo. The crude product was purified by preparative reversed-phase HPLC (eluent A: 0.1% TFA in H₂O and eluent B: ACN). Pure fractions were combined and lyophilized to afford Compound 169 (113 mg, 0.076 mmol, 28.1% yield) as a white solid. Analytical method 9; t_{R}=3.85 min; [M+H]⁺=1078.5, [M+2H]²⁺=539.7.

### Step 2. (2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-13-(4-(dimethylamino)butyl)-5-(methoxymethyl)-2,7,14-trimethyl-1,4,7,11,14-pentaazacyclooctadecane-3,6,10,15,18-pentaone hydrochloride (Compound 43)

To Compound 169 (42 mg, 0.030 mmol) dissolved in DMA (2 mL) was added 37% aq. formaldehyde (8.8 μL, 0.118 mmol) and sodium triacetoxyborohydride (25.1 mg, 0.118 mmol) and the reaction mixture was stirred for 4 h at rt. The reaction was quenched by the addition of H₂O (0.5 mL). The product was isolated by preparative reversed-phase HPLC (eluent A: 0.1% TFA in H₂O and eluent B: ACN). Pure fractions were combined and lyophilized. The product was dissolved in EtOAc (50 mL) and the organic layer was washed with 5% aq. Na₂CO₃ (3×4 mL) and brine (4 mL), dried over Na₂SO₄, filtered, and concentrated to dryness in vacuo. The residue was dissolved in ACN/H₂O (1:1) (20 mL) and 0.1 M aq. HCl (4 mL) was added. After lyophilization Compound 43 (24.5 mg, 0.019 mmol, 64.7% yield) was obtained as a white solid. Analytical method 9; t_{R}=3.91 min; [M+H]⁺=1106.5, [M+2H]²⁺=553.7.

The Compounds shown in Table 10 below were prepared according to the procedure used for Compound 43 (Example 3.5).

TABLE 10

| Cmd | | Synthesis | |
|---|---|---|---|
| No. | Structure | procedure | LCMS |
| 3 | 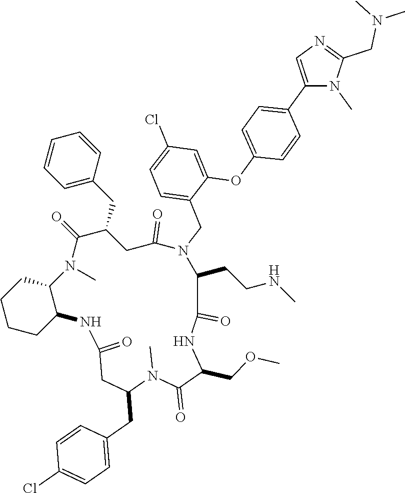 [SMILES: CNCC[C@H]1C(=O)N[C@@H](COC)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1] | Compound 43 (Example 3.5)- starting from Compound 109. | Analytical method 7 t_{R} = 0.93 min [M + 2H]²⁺ = 539.6 |
| 285 | 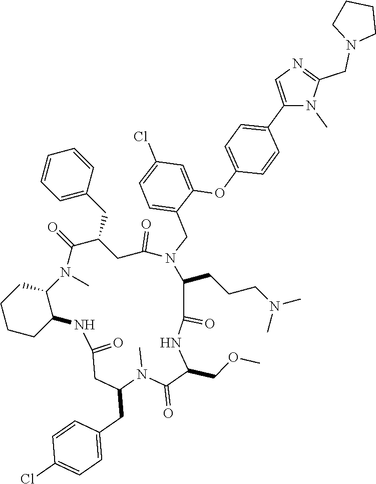 [SMILES: COC[C@@H]1NC(=O)[C@H](CCCN(C)C)N(Cc2ccc(Cl)cc2Oc2ccc(-c3cnc(CN4CCCC4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 43 (Example 3.5)-43 starting from intermediate 285A. | Analytical method 3 t_{R} = 0.91 min [M + H]⁺ = 1132.6 |

### Example 3.6: Synthesis of N-(4-((2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,14-trimethyl-3,6,10,15,18-pentaoxo-1,4,7,11,14-pentaazacyclooctadecan-13-yl)butyl)acetamide trifluoroacetate (Compound 217)

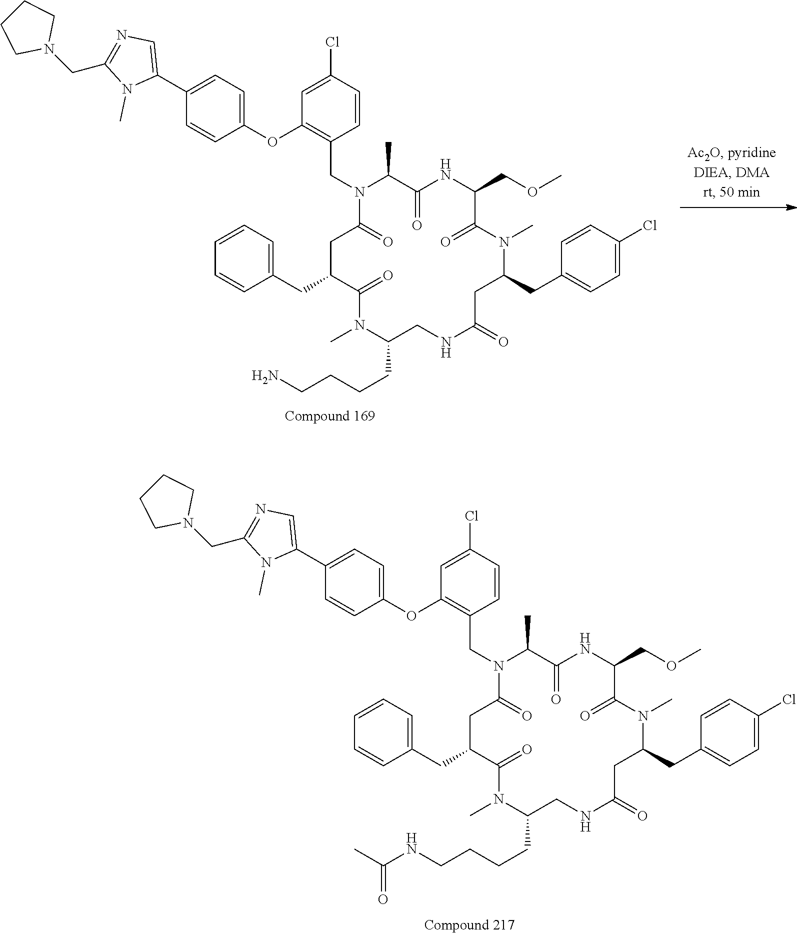 [SMILES: COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](CCCCN)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cnc(CN4CCCC4)n3C)cc2)C(=O)C[C@@H](Cc2ccccc2)C(=O)N(C)[C@@H](CCCCNC(C)=O)CNC(=O)C[C@H](Cc2ccc(Cl)cc2)N(C)C1=O]

To Compound 169 (15.0 mg, 10.55 μmol) dissolved in DMA (1 mL) was added Ac₂O (20 μL, 0.212 mmol), pyridine (10 μL, 0.124 mmol), and DIEA (10 μL, 0.057 mmol) and the resulting mixture was stirred for 50 min at rt. The reaction was quenched with AcOH (100 μL) and the product was isolated by preparative reversed-phase HPLC (eluent A: 0.1% TFA in H₂O and eluent B: ACN). Pure fractions were combined and lyophilized to afford Compound 217 (10.8 mg, 7.68 μmol, 72.8% yield) as a white solid. Analytical method 9; t_{R}=4.59 min; [M+H]⁺=1120.5, [M+2H]²⁺=560.7.

### Example 3.7: Synthesis of (2S,5S,8S,13R,16R)-16-benzyl-1-(4-chloro-2-(4-(2-((dimethylamino)methyl)-1-methyl-1H-imidazol-5-yl)phenoxy)benzyl)-8-(4-chlorobenzyl)-13-(hydroxymethyl)-5-(methoxymethyl)-2,7,14-trimethyl-1,4,7,11,14-pentaazacyclooctadecane-3,6,10,15,18-pentaone (Compound 235)

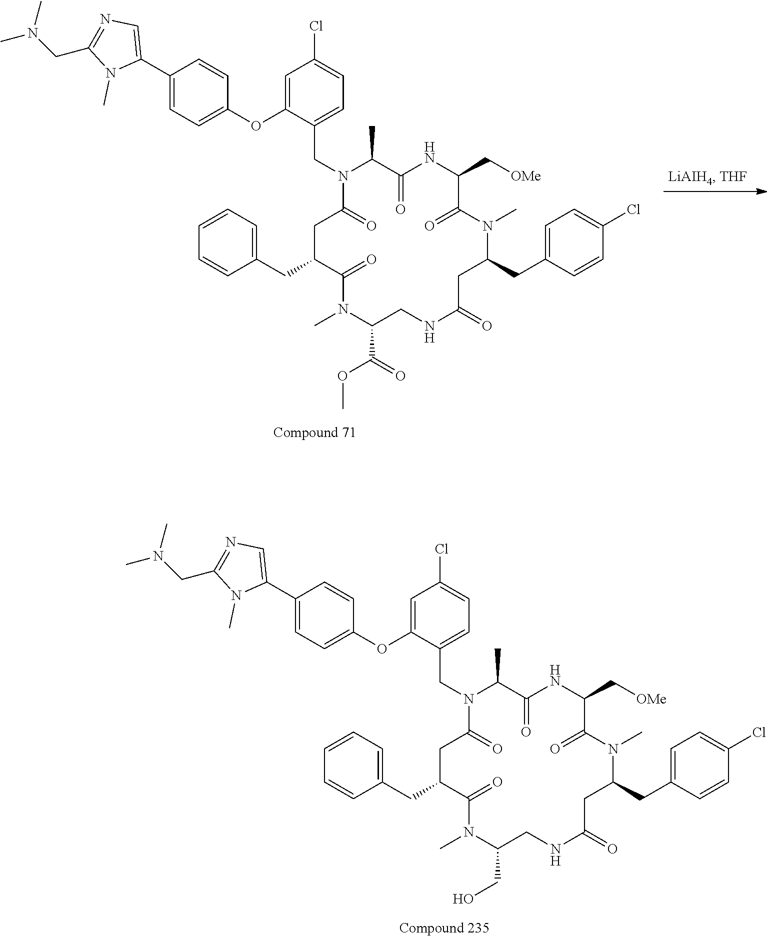 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H](C(=O)OC)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H](CO)CNC(=O)C[C@H](Cc2ccc(Cl)cc2)N(C)C1=O]

To Compound 71 (35 mg, 0.034 mmol) dissolved in dry THF (0.34 mL) and cooled to 0° C. was added lithium aluminum hydride in THF (1 M, 100 μL, 0.1 mmol) the resulting solution was stirred at 0° C. After 2 h, the reaction mixture was quenched with 1.0 M HCl and water, concentrated under reduced pressure, taken up in 3 mL EtOH, filtered, and purified by reversed-phase HPLC (MeCN/Water with 0.1% NH₄OH). Pure fractions were combined to obtain Compound 235 (3 mg, 8%) as a white powder. Analytical method 4; t_{R}=1.95 min, [M+2H]²⁺=506

### Example 3.8: Synthesis of 5-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-N-(piperidin-4-yl)picolinamide trifluoroacetate (Compound 122)

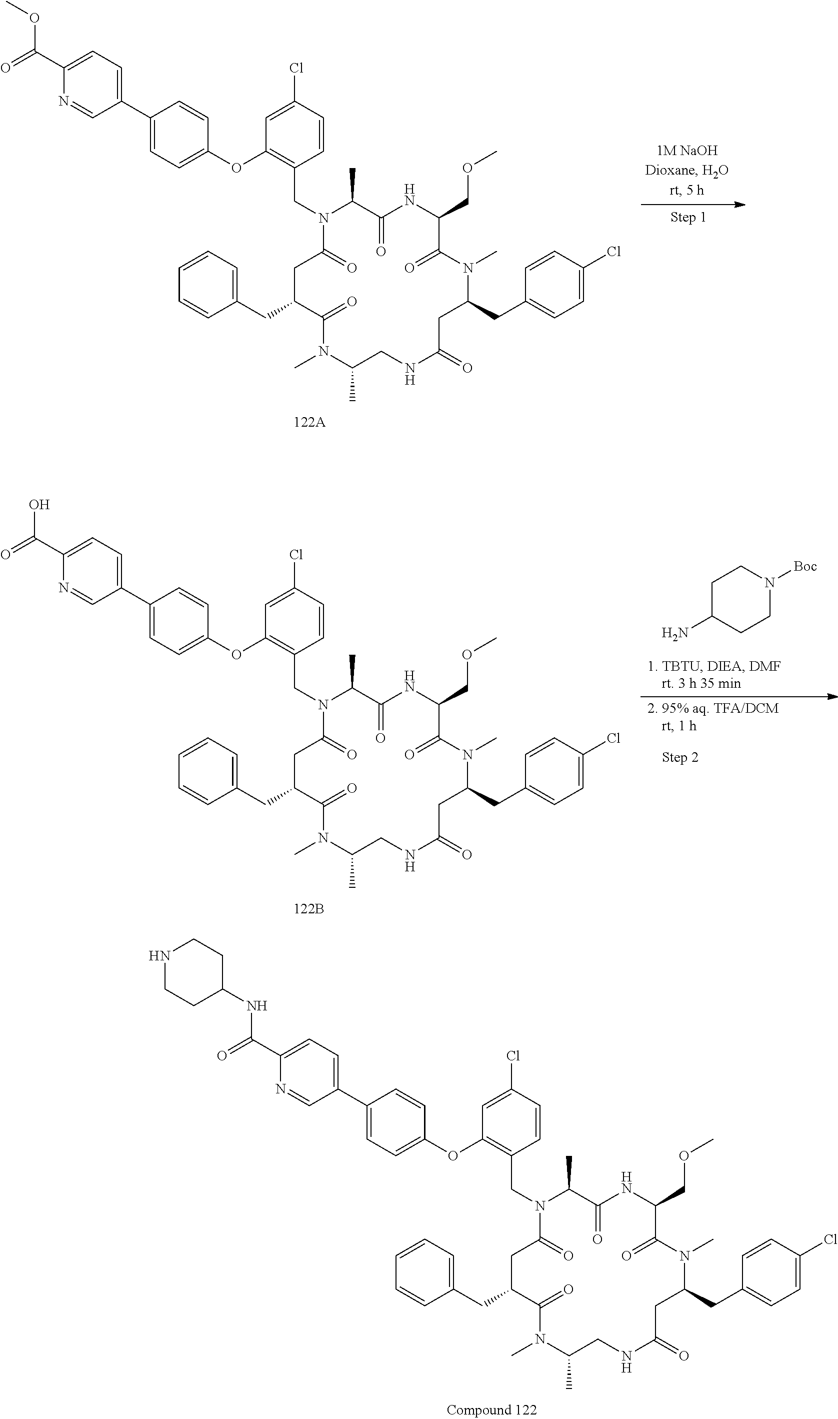 [SMILES: COC[C@@H]1NC(=O)[C@H](C)N(Cc2ccc(Cl)cc2Oc2ccc(-c3ccc(C(=O)OC)nc3)cc2)C(=O)C[C@@H](Cc2ccccc2)C(=O)N(C)[C@@H](C)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3ccc(C(=O)O)nc3)cc2)C(=O)C[C@@H](Cc2ccccc2)C(=O)N(C)[C@@H](C)CNC(=O)C[C@H](Cc2ccc(Cl)cc2)N(C)C1=O ; CC(C)(C)OC(=O)N1CCC(N)CC1 ; COC[C@@H]1NC(=O)[C@H](C)N(Cc2ccc(Cl)cc2Oc2ccc(-c3ccc(C(=O)NC4CCNCC4)nc3)cc2)C(=O)C[C@@H](Cc2ccccc2)C(=O)N(C)[C@@H](C)CNC(=O)C[C@H](Cc2ccc(Cl)cc2)N(C)C1=O]

### Step 1. 5-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)picolinic acid (122B)

To 122A (0.120 mmol) dissolved in dioxane/H₂O (9:1) (10 mL) was added 1 M NaOH (0.239 mL, 0.239 mmol). The resulting mixture was stirred for 5 h at rt and then partitioned between EtOAc (60 mL) and H₂O (10 mL). The organic layer was washed with H₂O (2×10 mL) and brine (10 mL), dried over Na₂SO₄, filtered, and concentrated to dryness in vacuo to afford 122B as a white solid. The crude product was used in the next step without purification. Analytical method 10; t_{R}=1.17 min; [M+H]⁺=979.5, [M−H]⁻=977.6.

### Step 2. 5-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-N-(piperidin-4-yl)picolinamide trifluoroacetate (Compound 122)

Step 2-1: 122B (60 μmol) and TBTU (23.12 mg, 72.0 μmol) were dissolved in DMF (5 mL) and DIEA (0.016 mL, 90 μmol). The solution was stirred for 2 min at rt, then 1-Boc-4-aminopiperidine (18.03 mg, 90 μmol) was added. The reaction was stirred for 3 h 35 min at rt, then partitioned between EtOAc (30 mL) and 5% aq. NaHCO₃ (5 mL). The organic layer was washed with 5% aq. NaHCO₃ (2×5 mL) and brine (5 mL), dried over Na₂SO₄, filtered, and concentrated to dryness in vacuo.

Step 2-2: The residue was dissolved in 95% aq. TFA/DCM (1:1) (5 mL). The reaction mixture was stirred for 1 h at rt, and then concentrated to dryness in vacuo. The crude product was purified by preparative HPLC (eluent A: 0.1% TFA in H₂O and eluent B: ACN). Pure fractions were combined and lyophilized to afford Compound 122 (33.8 mg, 25.9 μmol, 43.2% yield) as a white solid. Analytical method 12; t_{R}=4.59 min; [M+H]=1061.7, [M+2H]²⁺=531.3.

The Compounds shown in Table 11 were prepared according to the procedure used for Compound 122 (Example 3.8).

TABLE 11

| Cmd | | Synthesis | |
|---|---|---|---|
| No. | Structure | Procedure | LCMS |
| 113 | 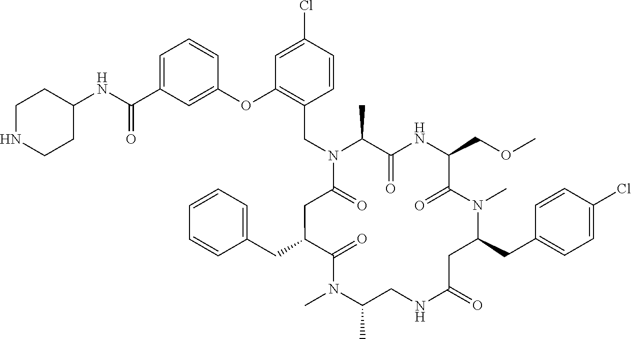 [SMILES: COC[C@@H]1NC(=O)[C@H](C)N(Cc2ccc(Cl)cc2Oc2cccc(C(=O)NC3CCNCC3)c2)C(=O)C[C@@H](Cc2ccccc2)C(=O)N(C)[C@@H](C)CNC(=O)C[C@H](Cc2ccc(Cl)cc2)N(C)C1=O] | Compound 122 (Example 3.8) | Analytical method 15 t_{R} = 2.21 min [M + H]⁺ = 984.4 |
| 177 | 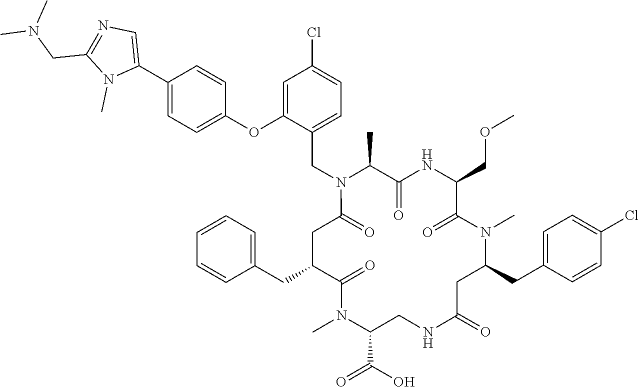 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccc2)C(=O)N(C)[C@@H](C(=O)O)CNC(=O)C[C@H](Cc2ccc(Cl)cc2)N(C)C1=O] | Compound 122 (Example 3.8) | Analytical method 4 t_{R} = 1.97 min [M + 2H]²⁺ = 513.3 |
| 244 | 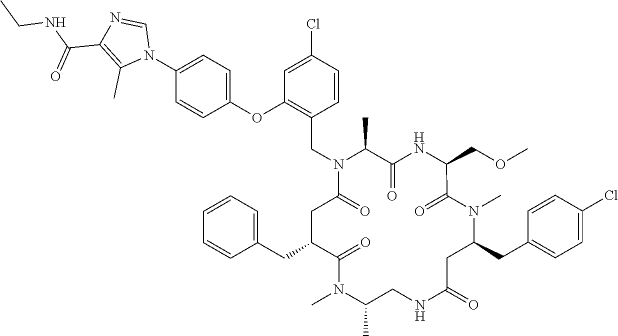 [SMILES: CCNC(=O)c1ncn(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)c1C] | Compound 122 (Example 3.8) | Analytical method 4 t_{R} = 2.83 min [M + 2H]²⁺ = 1009.3 |

### Example 3.9: Synthesis of 3-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)-N-(1-methylpiperidin-4-yl)benzamide (Compound 118)

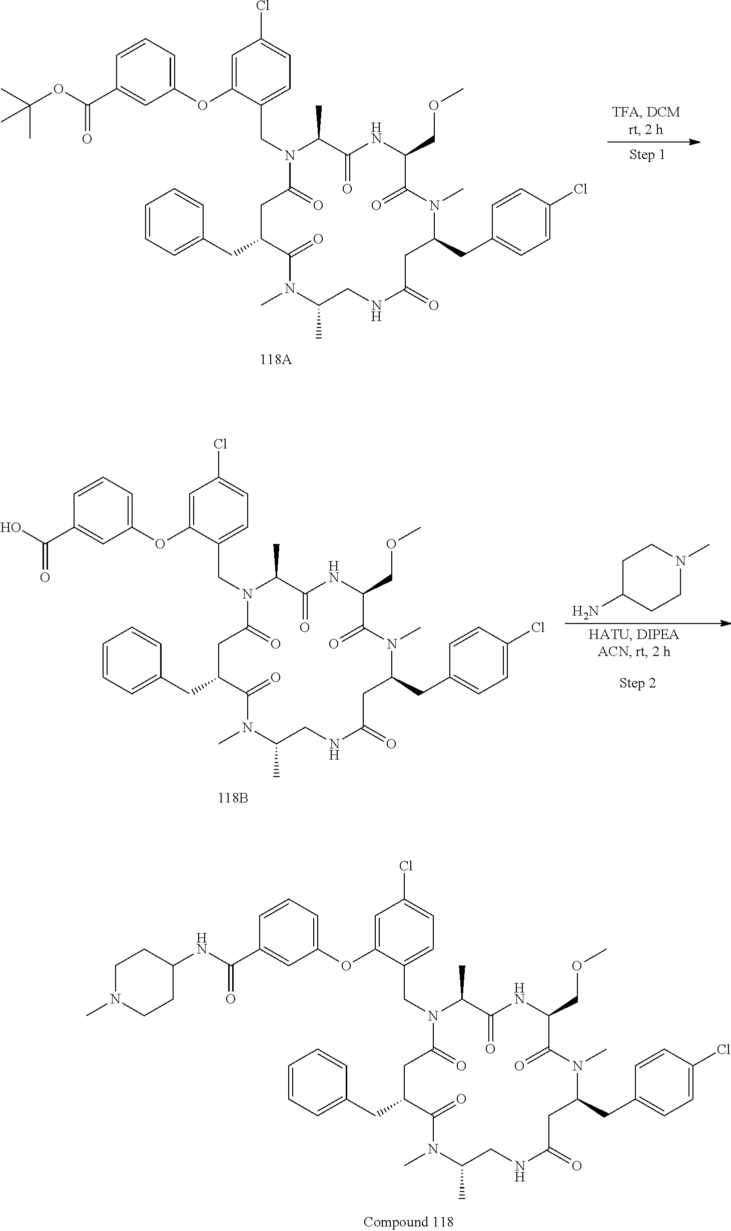

### Step 1. 3-(2-(((2S,5S,8S,13S,16R)-16-Benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)benzoic acid (118B)

To 118A (514 mg, 0.536 mmol) in DCM (10 mL) was added TFA (10 mL, 130 mmol) dropwise and the resulting mixture was stirred at rt for 2 h and then concentrated to dryness in vacuo. The crude mixture was lyophilized to afford 118B (562 mg, 0.622 mmol, 116% yield) as a white fluffy powder. The crude product carried onto the next step without purification. Analytical method 5; t_{R}=1.01 min; [M+H]⁺=902.5.

### Step 2. 3-(2-(((2S,5S,8S,13S,16R)-16-Benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)-N-(1-methylpiperidin-4-yl)benzamide (Compound 118)

To 118B (56 mg, 0.062 mmol) in ACN (2 mL) was added DIPEA (0.043 mL, 0.248 mmol) and 1-methylpiperidin-4-amine (14.17 mg, 0.124 mmol), followed by the addition of HATU (47.2 mg, 0.124 mmol). The resulting mixture was stirred at rt for 2 h. The crude was diluted with ACN (1 mL), filtered, and purified by HPLC (40→80% ACN in water, water contains 0.5 mM of NH₄OH) to afford Compound 118 (30 mg, 0.029 mmol, 47.4% yield) as a fluffy white powder after lyophilization. Analytical method 4; t_{R}=2.08 min; [M+H]⁺=998.9.

The Compounds shown in Table 12 were prepared according to the procedure used for Compound 118 or Intermediate 118B (Example 3.9).

TABLE 12

| Cmd | | Synthesis | |
|---|---|---|---|
| No. | Structure | Procedure | LCMS |
| 20 | 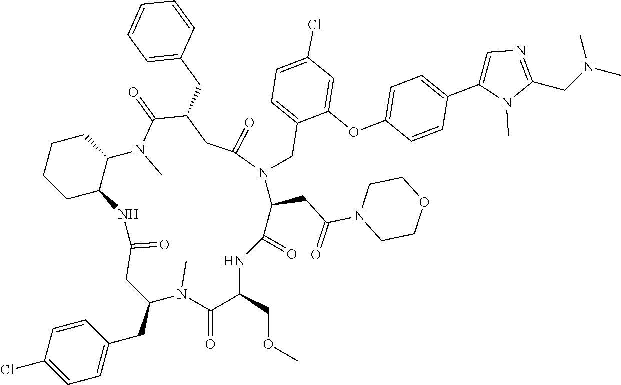 [SMILES: COC[C@@H]1NC(=O)[C@H](CC(=O)N2CCOCC2)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 118 (Example 3.9)- starting from Compound 219. | Analytical method 6 t_{R} = 2.33 min [M + H]⁺ = 1148.5 |
| 38 | 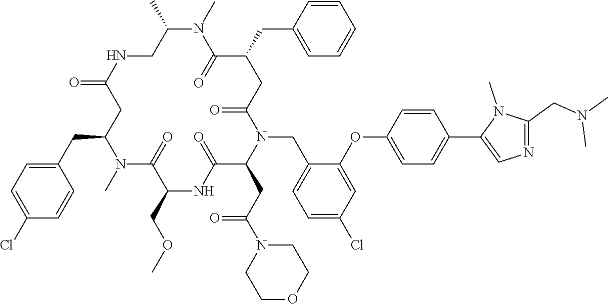 [SMILES: COC[C@@H]1NC(=O)[C@H](CC(=O)N2CCOCC2)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 118 (Example 3.9)- starting from Compound 82. | Analytical method 5 t_{R} = 1.13 min [M + H]⁺ = 1108.7 |
| 52 | 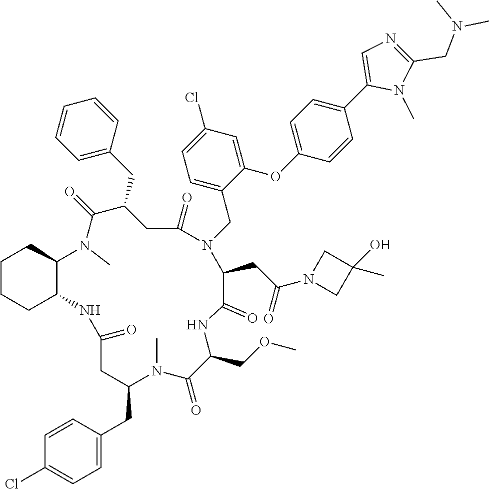 [SMILES: COC[C@@H]1NC(=O)[C@H](CC(=O)N2CC(C)(O)C2)N(Cc2ccc(Cl)cc2Oc2ccc(-c3cnc(CN(C)C)n3C)cc2)C(=O)C[C@@H](Cc2ccccc2)C(=O)N(C)[C@@H]2CCCC[C@H]2NC(=O)C[C@H](Cc2ccc(Cl)cc2)N(C)C1=O] | Compound 118 (Example 3.9)- starting from Compound 219. | Analytical method 6 t_{R} = 2.25 min [M + H]⁺ = 1148.5 |
| 82 | 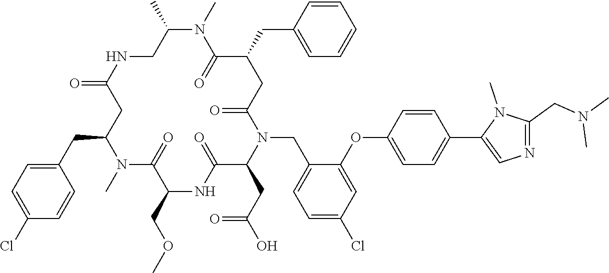 [SMILES: COC[C@@H]1NC(=O)[C@H](CC(=O)O)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 118B (Example 3.9)- starting from Compound 174. | Analytical method 3 t_{R} = 1.01 min [M + H]⁺ = 1039.4 |
| 93 | 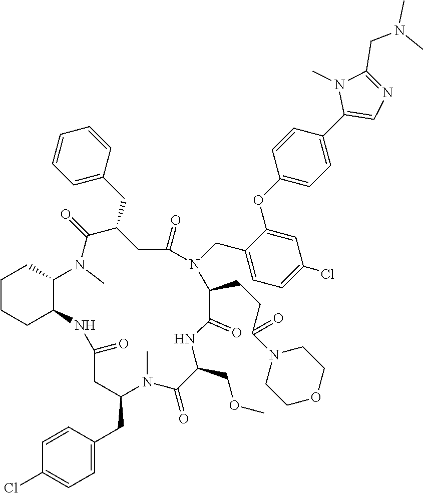 [SMILES: COC[C@@H]1NC(=O)[C@H](CCC(=O)N2CCOCC2)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 118 (Example 3.9)- starting from Compound 245. | Analytical method 6 t_{R} = 2.33 min [M + H]⁺ = 1162.5 |
| 95 | 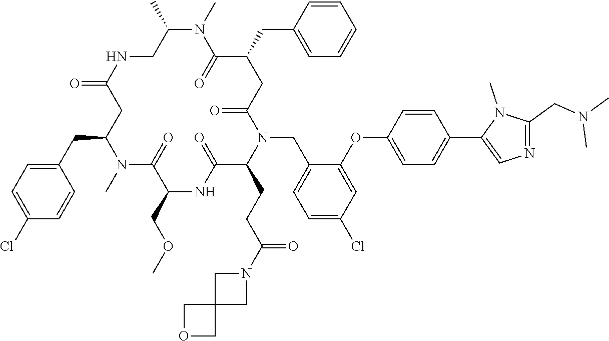 [SMILES: COC[C@@H]1NC(=O)[C@H](CCC(=O)N2CC3(COC3)C2)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 118 (Example 3.9)- starting from Compound 10. | Analytical method 3 t_{R} = 0.98 min [M + H]⁺ = 1134.5 |
| 106 | 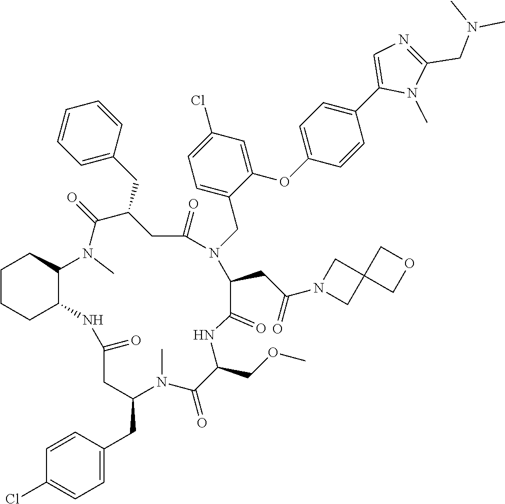 | Compound 118 (Example 3.9)- starting from Compound 219. | Analytical method 6 t_{R} = 2.26 min [M + H]⁺ = 1160.5 |
| 160 | 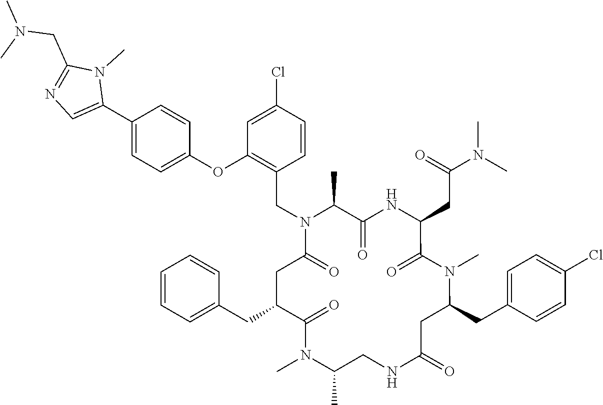 [SMILES: C[C@H]1CNC(=O)C[C@H](Cc2ccc(Cl)cc2)N(C)C(=O)[C@H](CC(=O)N(C)C)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Compound 118 (Example 3.9)- starting from Compound 213. | Analytical method 7 t_{R} = 1.03 min [M + H]⁺ = 1036.4 |
| 175 | 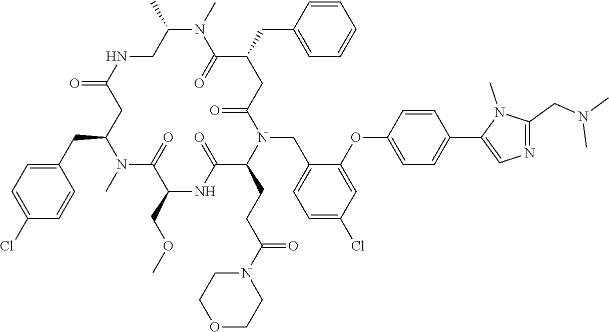 [SMILES: COC[C@@H]1NC(=O)[C@H](CCC(=O)N2CCOCC2)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 118 (Example 3.9)- starting from Compound 10. | Analytical method 3 t_{R} = 1.01 min [M + H]⁺ = 1122.5 |
| 213 | 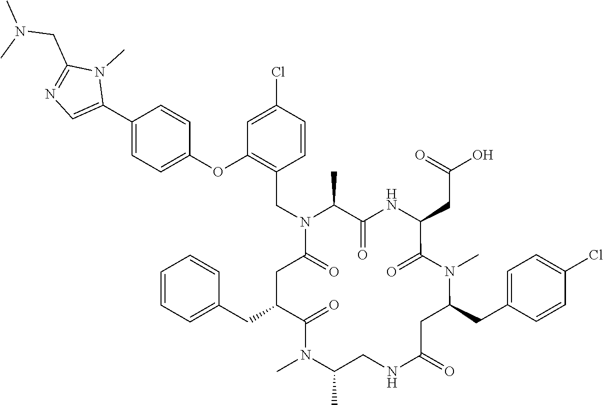 [SMILES: C[C@H]1CNC(=O)C[C@H](Cc2ccc(Cl)cc2)N(C)C(=O)[C@H](CC(=O)O)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Compound 118B (Example 3.9)- starting from Compound 185. | Analytical method 6 t_{R} = 1.99 min [M + H]⁺ = 1009.4 |
| 218 | 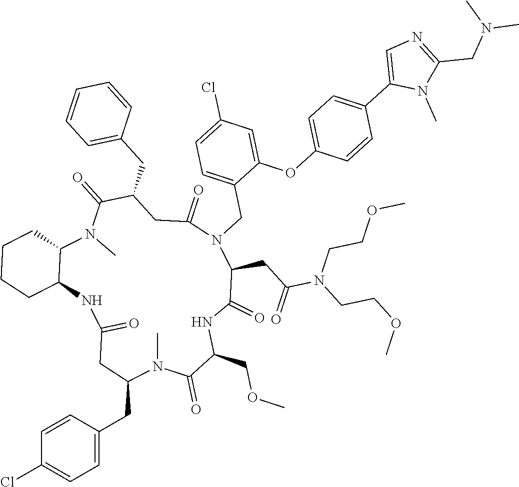 [SMILES: COCCN(CCOC)C(=O)C[C@H]1C(=O)N[C@@H](COC)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1] | Compound 118 (Example 3.9)- starting from Compound 219. | Analytical method 6 t_{R} = 2.43 min [M + H]⁺ = 1194.9 |
| 219 | 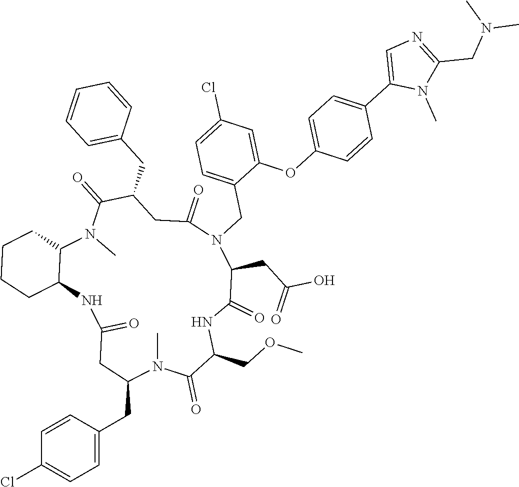 | Compound 118B (Example 3.9)- starting from Compound 237. | Analytical method 6 t_{R} = 2.31 min [M + H]⁺ = 1079.5 |
| 233 | 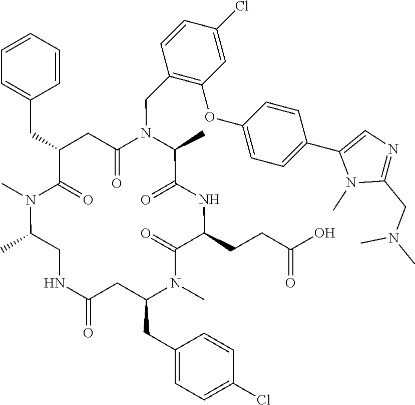 [SMILES: C[C@H]1CNC(=O)C[C@H](Cc2ccc(Cl)cc2)N(C)C(=O)[C@H](CCC(=O)O)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C] | Compound 118B (Example 3.9)- starting from Compound 233A. | Analytical method 4 t_{R} = 1.97 min [M + H]⁺ = 1023.2 |
| 239 | 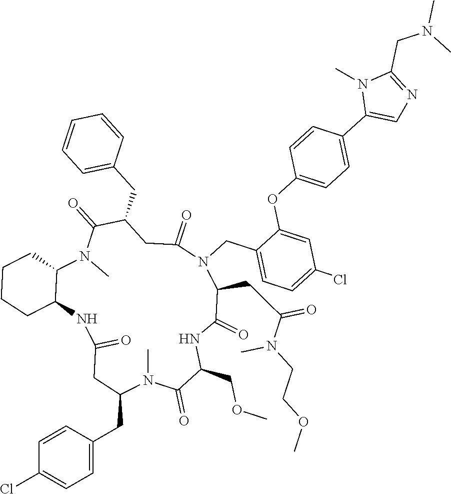 [SMILES: COCCN(C)C(=O)C[C@H]1C(=O)N[C@@H](COC)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1] | Compound 118 (Example 3.9)- starting from Compound 219. | Analytical method 6 t_{R} = 2.38 min [M + H]⁺ = 1150.5 |
| 245 | 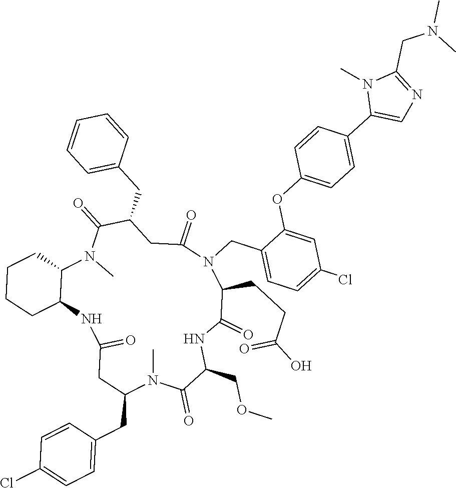 [SMILES: COC[C@@H]1NC(=O)[C@H](CCC(=O)O)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 118 (Example 3.9)- starting from Compound 46. | Analytical method 3 t_{R} = 1.11 min [M + H]⁺ = 1093.5 |

### Example 3.10: Synthesis of (2S,5S,8S,13S,16R)-16-benzyl-1-(4-chloro-2-(4-(5-(4-((2-fluoroethyl)amino)cyclohexyl)-pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone hydrochloride (Compound 36)

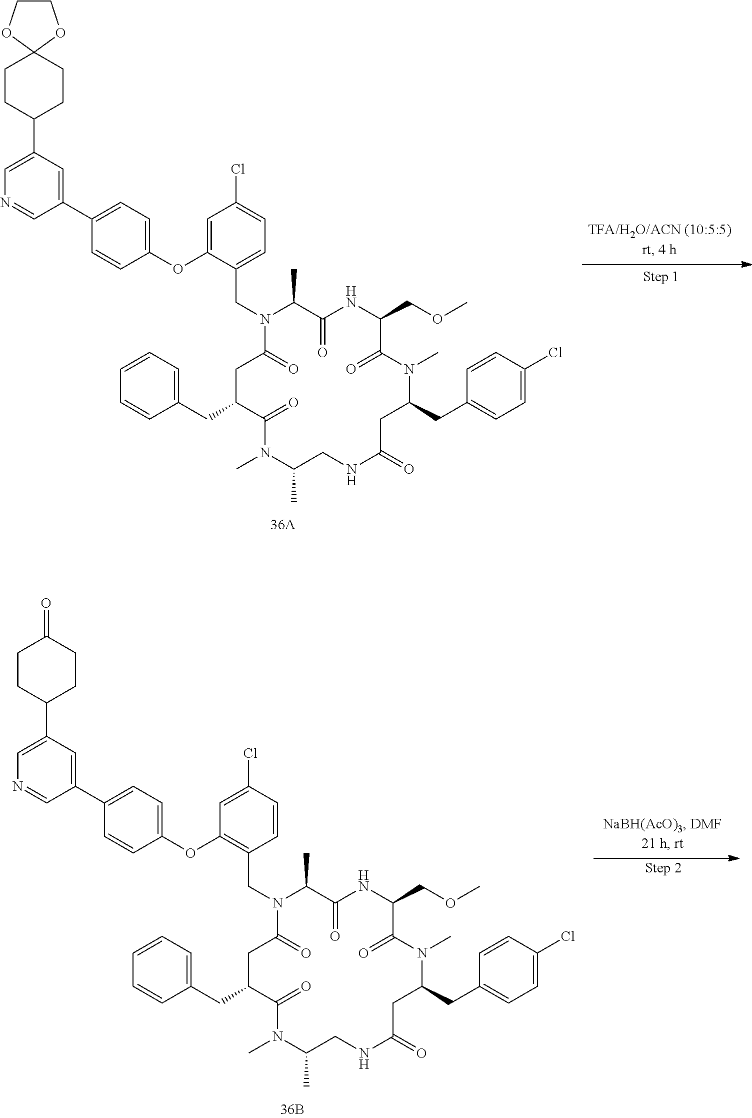 [SMILES: COC[C@@H]1NC(=O)[C@H](C)N(Cc2ccc(Cl)cc2Oc2ccc(-c3cncc(C4CCC5(CC4)OCCO5)c3)cc2)C(=O)C[C@@H](Cc2ccccc2)C(=O)N(C)[C@@H](C)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cncc(C4CCC(=O)CC4)c3)cc2)C(=O)C[C@@H](Cc2ccccc2)C(=O)N(C)[C@@H](C)CNC(=O)C[C@H](Cc2ccc(Cl)cc2)N(C)C1=O]

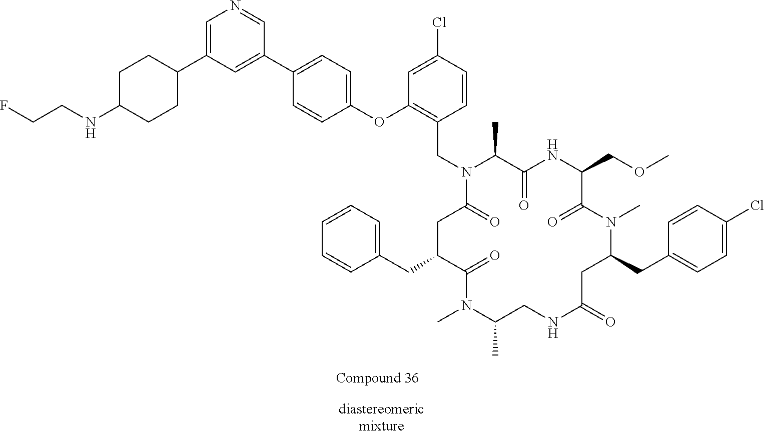 [SMILES: COC[C@@H]1NC(=O)[C@H](C)N(Cc2ccc(Cl)cc2Oc2ccc(-c3cncc(C4CCC(NCCF)CC4)c3)cc2)C(=O)C[C@@H](Cc2ccccc2)C(=O)N(C)[C@@H](C)CNC(=O)C[C@H](Cc2ccc(Cl)cc2)N(C)C1=O]

### Step 1. (2S,5S,8S,13S,16R)-16-Benzyl-1-(4-chloro-2-(4-(5-(4-oxocyclohexyl)pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone (36B)

36A (62.1 mg, 0.052 mmol) was dissolved in TFA/H₂O/ACN (10:5:5) (30 mL). The reaction mixture was stirred for 1 h at rt, and then concentrated to dryness in vacuo. The residue was lyophilized from ACN/H₂O (1:1) and again dissolved in TFA/H₂O/ACN (50:25:25) (65 mL). The resulting mixture was stirred for 3 h at rt, and then concentrated to dryness in vacuo. The residue was lyophilized from ACN/H₂O (1:1) to give 36B, which was carried onto the next step without purification. Analytical method 10; t_{R}=1.30 min; [M+H]⁺=1031.7.

### Step 2. 4. (2S,5S,8S,13S,16R)-16-Benzyl-1-(4-chloro-2-(4-(5-(4-((2-fluoroethyl)amino)cyclohexyl)-pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone hydrochloride (Compound 36)

To half of the crude material (36B, calculated with 26 μmol) dissolved in DMF (12 mL) was added 2-Fluoroethanamine HCl (9.12 mg, 0.092 mmol) and DIEA (0.016 mL, 0.092 mmol) and the resulting mixture was stirred for 5 min. NaBH(AcO)₃ (19.43 mg, 0.092 mmol) was then added and the mixture was stirred for 21 h at rt and then concentrated in vacuo. The crude product was purified by preparative reversed-phase HPLC (eluent A: 0.1% TFA in H₂O and eluent B: ACN). Pure fractions were combined and lyophilized to yield the product as a trifluoroacetate salt. The product was partitioned between EtOAc (30 mL) and 5% aq. NaHCO₃ (5 mL). The organic layer was washed with 5% aq. NaHCO₃ (5 mL) and brine (5 mL), dried over Na₂SO₄, filtered, and concentrated to dryness in vacuo. The residue was dissolved in ACN/H₂O (1:1) (20 mL) and a solution of 1 M aq. HCl (64 μL, 64 μmol) in H₂O (5 mL) was added. After lyophilization Compound 36 (diastereomeric mixture) (11.2 mg, 8.85 μmol) was obtained as a white solid. Analytical method 13; t_{R}=3.89 min; [M+H]⁺=1078.5.

### Example 3.11: Synthesis of 3-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-5,6-dihydro-8H-spiro[imidazo[1,2-a]pyrazine-7,1′-pyrrolidin]-7-ium chloride hydrochloride (Compound 249)

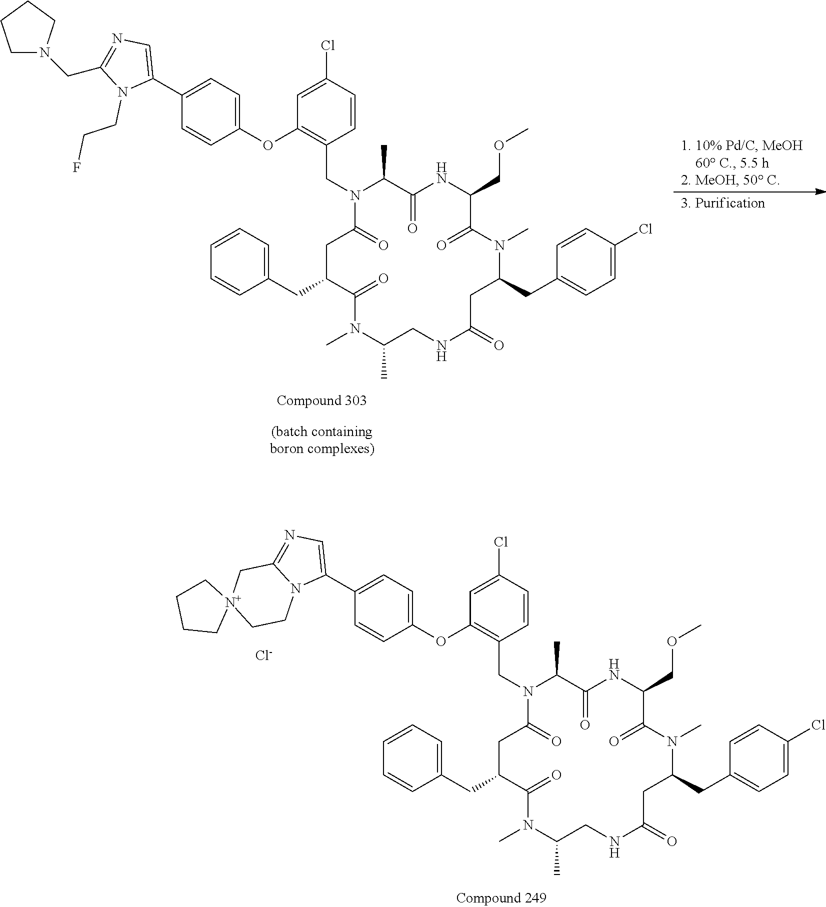 [SMILES: COC[C@@H]1NC(=O)[C@H](C)N(Cc2ccc(Cl)cc2Oc2ccc(-c3cnc(CN4CCCC4)n3CCF)cc2)C(=O)C[C@@H](Cc2ccccc2)C(=O)N(C)[C@@H](C)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cnc4n3CC[N+]3(CCCC3)C4)cc2)C(=O)C[C@@H](Cc2ccccc2)C(=O)N(C)[C@@H](C)CNC(=O)C[C@H](Cc2ccc(Cl)cc2)N(C)C1=O ; [Cl-]]

To Compound 303 (˜96 μmol) (containing boron complexes deriving from the use of sodium cyanoborohydride in the reductive alkylation step (Example 2.1, Step 5) dissolved in MeOH (5 mL) was added 10% Pd/C (102 mg, 96 μmol). The resulting suspension was stirred at 60° C. for 5.5 h and filtered through Hyflo. The resulting solution was then stirred at 60° C. for 89.5 h and concentrated to dryness in vacuo. The crude product was purified by preparative reversed-phase HPLC (eluent A: 0.1% TFA in H₂O and eluent B: ACN). Pure fractions were combined and lyophilized to afford Compound 249 as a TFA salt. The solid was partitioned between EtOAc (30 mL) and 1 M NaOH (2 mL). The organic layer was washed with 1 M NaOH (2×2 mL) and brine (3 mL), dried over Na₂SO₄, filtered, and concentrated to dryness in vacuo. The crude product was dissolved in ACN/H₂O (1:1) (20 mL) and 1 M HCl (93 μL) and then lyophilized to afford Compound 249 (23.3 mg, 20.42 μmol, 21.3% yield) as a white solid which was carried onto the next step without purification. Analytical method 13; t_{R}=4.06 min; [M]⁺=1033.5, [M+H]²⁺=517.2.

### Example 3.12: Synthesis of (2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2-(hydroxymethyl)-5-(methoxymethyl)-7,13,14-trimethyl-1,4,7,11,14-pentaazacyclooctadecane-3,6,10,15,18-pentaone (Compound 11)

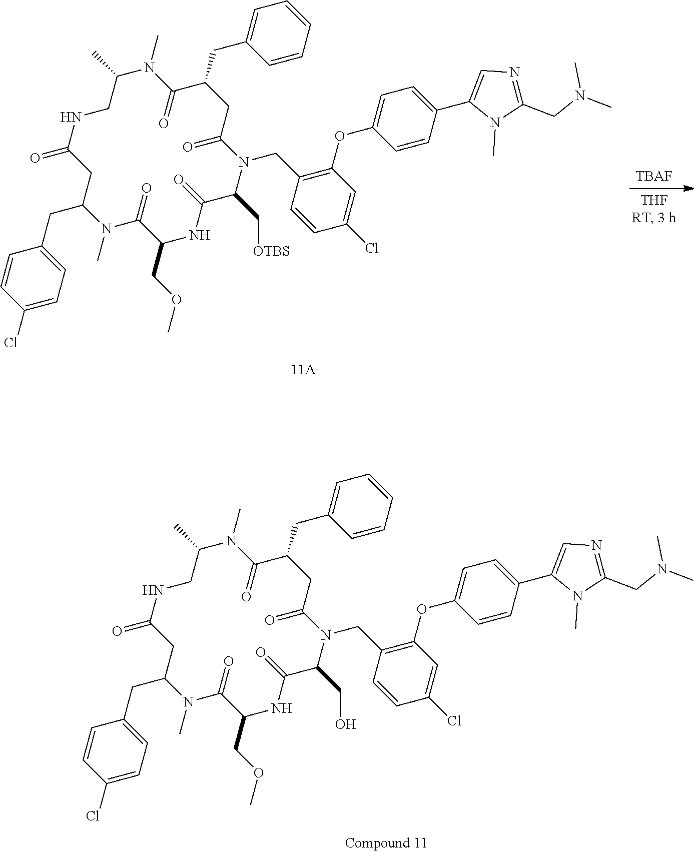

Intermediate 11A (0.187 mmol) was taken up in TBAF solution (1 M in THF, 1.9 mL, 1.90 mmol). The resulting mixture was stirred for 3 h at rt. The mixture was concentrated, then purified directly via reversed-phase chromatography (MeCN/water with 0.1% NH₄OH) to afford 126 mg of a white solid. The solid was further purified by reversed-phase HPLC (MeCN/water with 0.1% NH₄OH) and clean fractions were pooled and lyophilized to yield Compound 11 (42 mg, 22%). Analytical method 3; t_{R}=1.10 min; [M+H]⁺=1011.4.

The Compounds in shown in Table 13 were prepared according to the procedure used for Compound 11 (Example 3.12).

TABLE 13

| Cmp | | Synthesis | |
|---|---|---|---|
| No. | Structure | Procedure | LCMS |
| 66 | 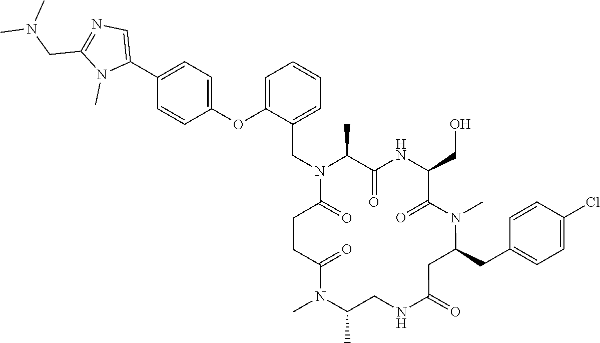 [SMILES: C[C@H]1CNC(=O)C[C@H](Cc2ccc(Cl)cc2)N(C)C(=O)[C@H](CO)NC(=O)[C@H](C)N(Cc2ccccc2Oc2ccc(-c3cnc(CN(C)C)n3C)cc2)C(=O)CCC(=O)N1C] | Compound 11 (Example 3.12) | Analytical method 2 t_{R} = 2.20 min [M + H]⁺ = 857.3 |
| 75 | 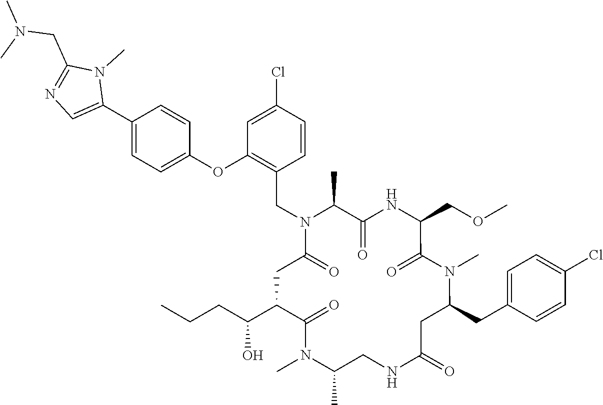 [SMILES: CCC[C@@H](O)[C@@H]1CC(=O)N(Cc2ccc(Cl)cc2Oc2ccc(-c3cnc(CN(C)C)n3C)cc2)[C@@H](C)C(=O)N[C@@H](COC)C(=O)N(C)[C@@H](Cc2ccc(Cl)cc2)CC(=O)NC[C@H](C)N(C)C1=O] | Compound 11 (Example 3.12) | Analytical method 2 t_{R} = 2.57 min [M + H]⁺ = 977.5 |
| 143 | 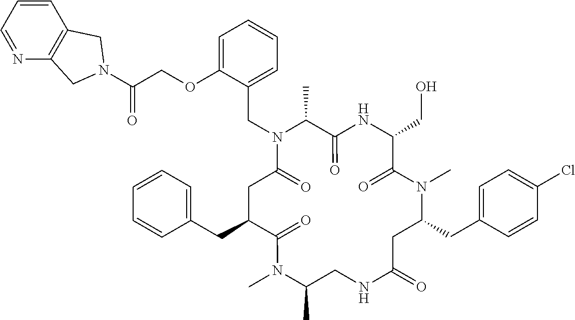 [SMILES: C[C@@H]1CNC(=O)C[C@@H](Cc2ccc(Cl)cc2)N(C)C(=O)[C@@H](CO)NC(=O)[C@@H](C)N(Cc2ccccc2OCC(=O)N2Cc3cccnc3C2)C(=O)C[C@H](Cc2ccccc2)C(=O)N1C] | Compound 11 (Example 3.12) | Analytical method 3 t_{R} = 1.21 min [M + H]⁺ = 894.4 |
| 151 | 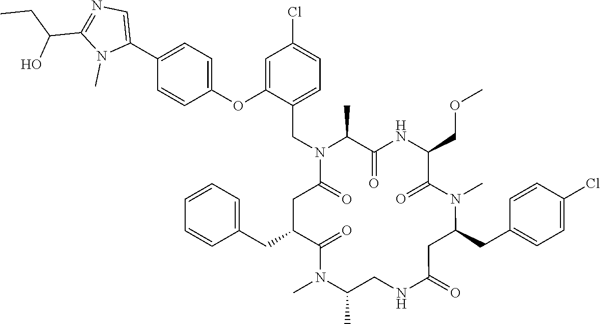 [SMILES: CCC(O)c1ncc(-c2ccc(Oc3cc(Cl)ccc3CN3C(=O)C[C@@H](Cc4ccccc4)C(=O)N(C)[C@@H](C)CNC(=O)C[C@H](Cc4ccc(Cl)cc4)N(C)C(=O)[C@H](COC)NC(=O)[C@@H]3C)cc2)n1C] | Compound 11 (Example 3.12) | Analytical method 4 t_{R} = 2.09 min [M + H]⁺ = 994.4 |
| 167 | 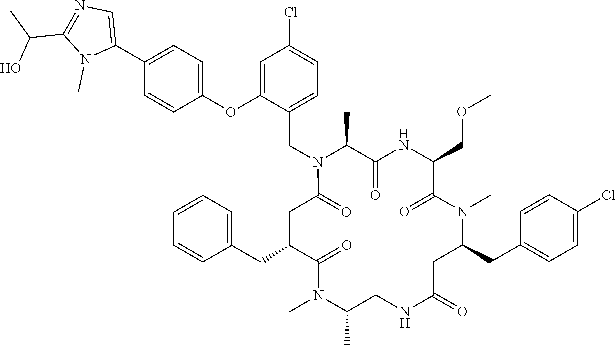 [SMILES: COC[C@@H]1NC(=O)[C@H](C)N(Cc2ccc(Cl)cc2Oc2ccc(-c3cnc(C(C)O)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 11 (Example 3.12) | Analytical method 8 t_{R} = 1.42 min [M + H]⁺ = 982.5 |
| 200 | 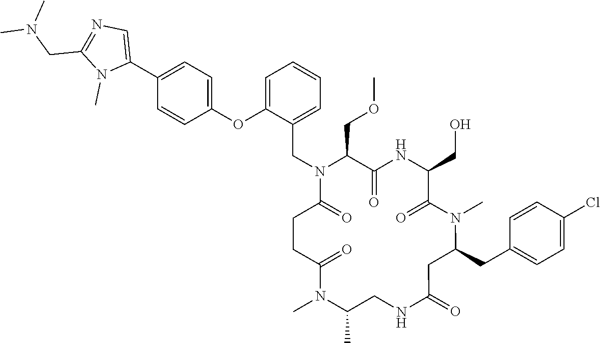 [SMILES: COC[C@H]1C(=O)N[C@@H](CO)C(=O)N(C)[C@@H](Cc2ccc(Cl)cc2)CC(=O)NC[C@H](C)N(C)C(=O)CCC(=O)N1Cc1ccccc1Oc1ccc(-c2cnc(CN(C)C)n2C)cc1] | Compound 11 (Example 3.12) | Analytical method 4 t_{R} = 1.60 min [M + H]⁺ = 887.3 |
| 207 | 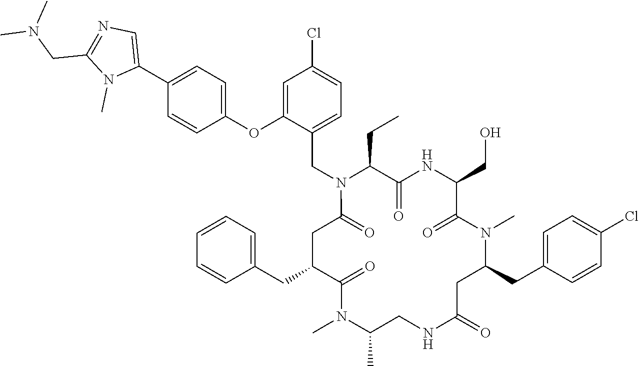 [SMILES: CC[C@H]1C(=O)N[C@@H](CO)C(=O)N(C)[C@@H](Cc2ccc(Cl)cc2)CC(=O)NC[C@H](C)N(C)C(=O)[C@H](Cc2ccccc2)CC(=O)N1Cc1ccc(Cl)cc1Oc1ccc(-c2cnc(CN(C)C)n2C)cc1] | Compound 11 (Example 3.12) | Analytical method 2 t_{R} = 2.74 min [M + 2H]²⁺ = 498.4 |
| 242 | 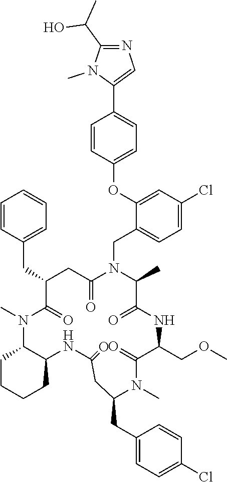 [SMILES: COC[C@@H]1NC(=O)[C@H](C)N(Cc2ccc(Cl)cc2Oc2ccc(-c3cnc(C(C)O)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 11 (Example 3.12) | Analytical method 2 t_{R} = 2.85 min [M + H]⁺ = 1022.3 |
| 260 | 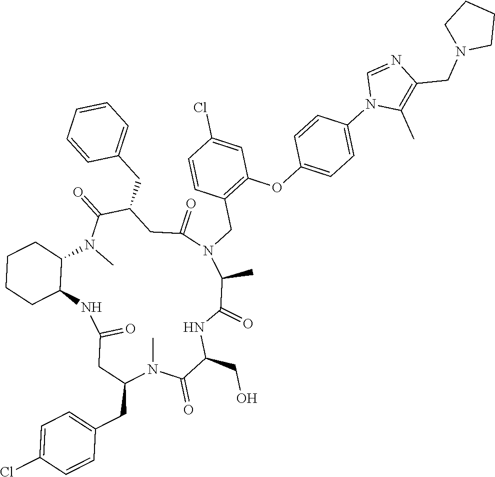 [SMILES: Cc1c(CN2CCCC2)ncn1-c1ccc(Oc2cc(Cl)ccc2CN2C(=O)C[C@@H](Cc3ccccc3)C(=O)N(C)[C@H]3CCCC[C@@H]3NC(=O)C[C@H](Cc3ccc(Cl)cc3)N(C)C(=O)[C@H](CO)NC(=O)[C@@H]2C)cc1] | Compound 11 (Example 3.12) | Analytical method 4 t_{R} = 2.10 min [M + H]⁺ = 1046.6 |
| 278 | 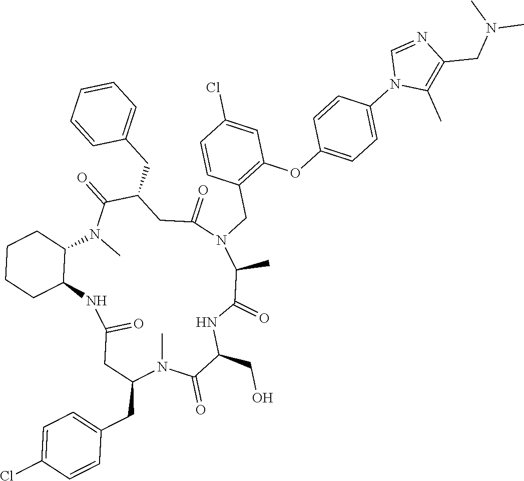 [SMILES: Cc1c(CN(C)C)ncn1-c1ccc(Oc2cc(Cl)ccc2CN2C(=O)C[C@@H](Cc3ccccc3)C(=O)N(C)[C@H]3CCCC[C@@H]3NC(=O)C[C@H](Cc3ccc(Cl)cc3)N(C)C(=O)[C@H](CO)NC(=O)[C@@H]2C)cc1] | Compound 11 (Example 3.12) | Analytical method 3 t_{R} = 1.04 min [M + H]⁺ = 1021.5 |

### Example 3.13: Synthesis of (2S,5S,8S,13S,16R)-16-benzyl-1-(4-chloro-2-(4-(2-(1-hydroxyethyl)-1-methyl-1H-imidazol-5-yl)phenoxy)benzyl)-8-(4-chlorobenzyl)-5-(hydroxymethyl)-2,7,13,14-tetramethyl-1,4,7,11,14-pentaazacyclooctadecane-3,6,10,15,18-pentaone (Compound 21)

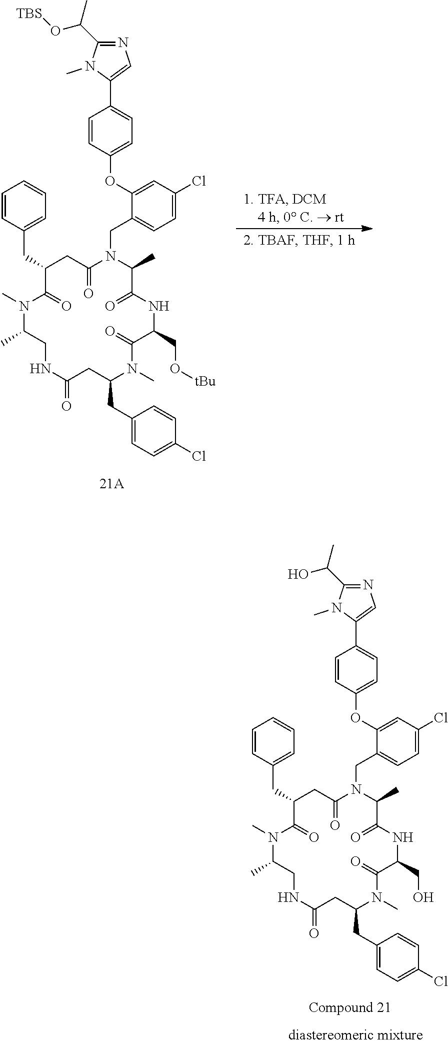

To a solution of 21A (75 mg, 0.066 mmol) in anhydrous DCM (3.0 mL) at 0° C. was added TFA (3 mL) dropwise. The resulting mixture was stirred at 0° C. under an atmosphere of nitrogen for 2 h, and then at rt for 2 h. The reaction mixture was concentrated via rotovap to remove the TFA and solvent. The resulting oil was dissolved in 5 mL of EtOAc, washed with 5.5% W/W NaHCO₃, dried over sodium sulfate, filtered, and concentrated via rotary evaporation. The resulting oil was treated with 1.0 M TBAF in THF (1.975 mL, 1.975 mmol) and stirred at rt for 1 h. The reaction was quenched with 10 mL of 5.5% W/W NaHCO₃ and extracted with 2×10 mL of EtOAc. The combined organic layers were purified by reverse phase HPLC (Phenomenex Gemini 5 m C18, 30×100 mm column, eluting with 30→80% ACN in water, 5 mM NH₄OH in water only) to afford Compound 21 (35.5 mg, 0.037 mmol, 55.7% yield) after lyophilization. Analytical method 5; t_{R}=1.16 min; [M+H]⁺=968.4.

### Example 3.14: Synthesis of (4S,7S,10S,14R,16aS,20aS)-14-benzyl-11-(4-chloro-2-(4-(2-((dimethylamino)-methyl)-1-methyl-1H-imidazol-5-yl)phenoxy)benzyl)-4-(4-chlorobenzyl)-7-(methoxymethyl)-5,16-dimethyl-10-(2-morpholinoethyl)hexadecahydrobenzo-[l][1,4,7,11,14]pentaazacycloocta-decine-2,6,9,12,15(3H)-pentaone (Compound 101)

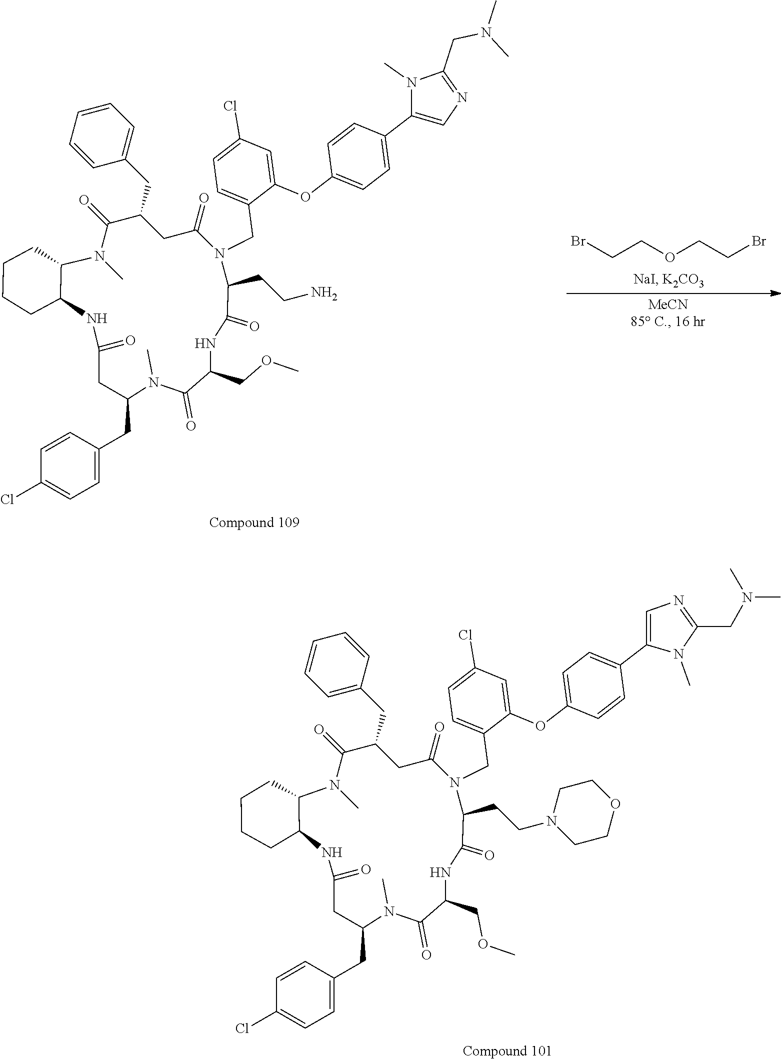 [SMILES: COC[C@@H]1NC(=O)[C@H](CCN)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O ; BrCCOCCBr ; COC[C@@H]1NC(=O)[C@H](CCN2CCOCC2)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O]

To a solution of Compound 109 (20 mg, 0.019 mmol), K₂CO₃ (5.19 mg, 0.038 mmol) and NaI (4.22 mg, 0.028 mmol) in MeCN (2 mL, dry) was added bis(2-bromoethyl) ether (4.35 μL, 0.028 mmol) and the resulting mixture was heated to 85° C. overnight. The reaction mixture was filtered through syringe and then directly purified via reversed-phase HPLC (eluting with 60→75% MeCN in water, 0.1% NH₄OH, 10 min gradient, XBridge Peptide BEH C18 Sum 19×150 mm). The clean fractions containing product were collected and lyophilized to yield Compound 101 (2.6 mg, 11%). Analytical method 7; t_{R}=0.90 min; [M+H]⁺=1134.7.

The Compounds shown in Table 14 below were prepared according to the procedure used for Compound 101 (Example 3.14).

TABLE 14

| Cmp | | Synthesis | |
|---|---|---|---|
| No. | Structure | Procedure | LCMS |
| 158 | 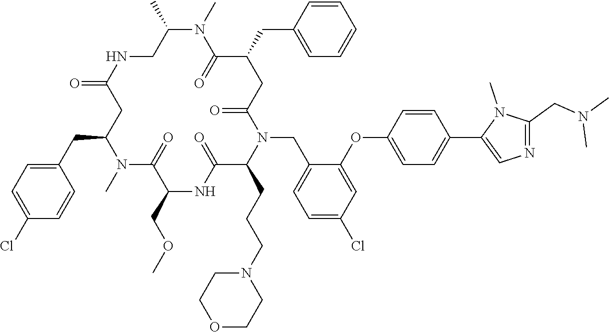 [SMILES: COC[C@@H]1NC(=O)[C@H](CCCN2CCOCC2)N(Cc2ccc(Cl)cc2Oc2ccc(-c3cnc(CN(C)C)n3C)cc2)C(=O)C[C@@H](Cc2ccccc2)C(=O)N(C)[C@@H](C)CNC(=O)C[C@H](Cc2ccc(Cl)cc2)N(C)C1=O] | Compound 101 (Example 3.14)- starting from Compound 192. | Analytical method 3 t_{R} = 0.86 min [M + H]⁺ = 1108.5 |
| 251 | 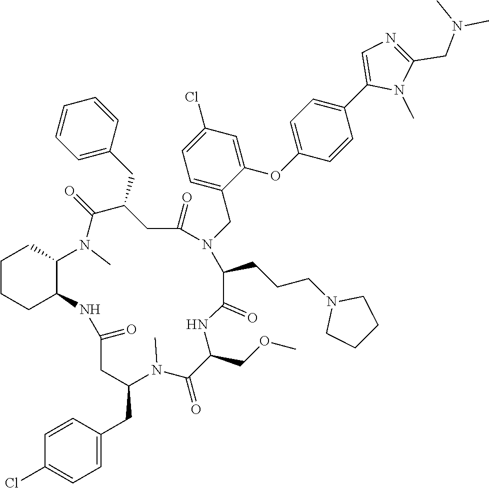 [SMILES: COC[C@@H]1NC(=O)[C@H](CCCN2CCCC2)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 101 (Example 3.14)- starting from Compound 304. | Analytical method 3 t_{R} = 0.91 min [M + H]⁺ = 1132.7 |
| 252 | 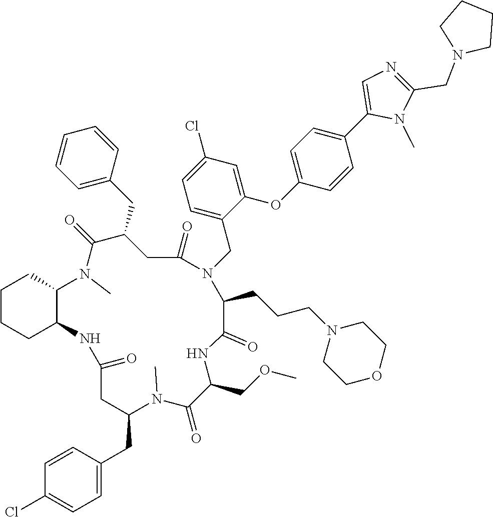 [SMILES: COC[C@@H]1NC(=O)[C@H](CCCN2CCOCC2)N(Cc2ccc(Cl)cc2Oc2ccc(-c3cnc(CN4CCCC4)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 101 (Example 3.14)- starting from Compound 252A. | Analytical method 3 t_{R} = 0.91 min [M + H]⁺ = 1174.6 |
| 267 | 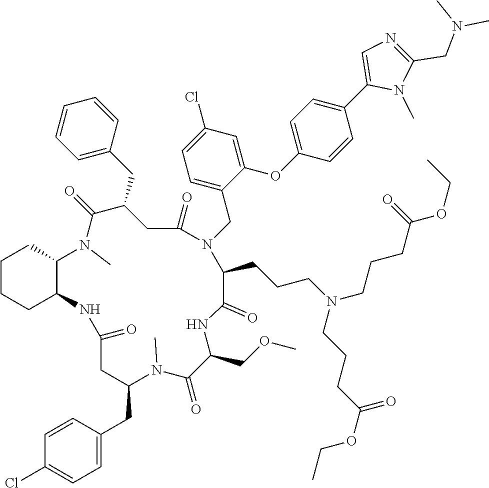 [SMILES: CCOC(=O)CCCN(CCCC(=O)OCC)CCC[C@H]1C(=O)N[C@@H](COC)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1] | Compound 101 (Example 3.14)- starting from Compound 304 | Analytical method 3 t_{R} = 0.98 min [M + H]⁺ = 1306.7 |
| 272 | 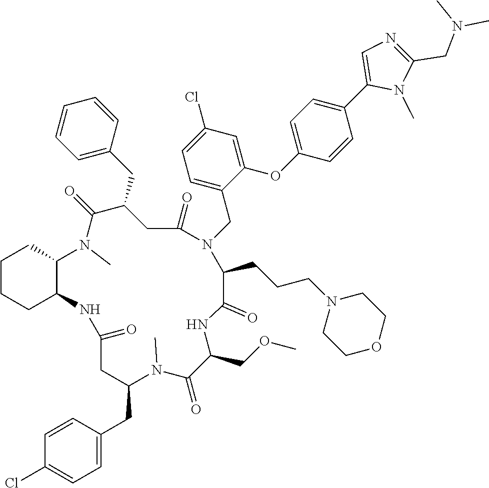 [SMILES: COC[C@@H]1NC(=O)[C@H](CCCN2CCOCC2)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O] | Compound 101 (Example 3.14)- starting from Compound 304. | Analytical method 3 t_{R} = 0.90 min [M + H]⁺ = 1148.6 |

### Example 3.15: Synthesis of (4S,7S,10S,14R,16aS,20aS)-14-benzyl-11-(4-chloro-2-(4-(2-((dimethylamino)-methyl)-1-methyl-1H-imidazol-5-yl)phenoxy)benzyl)-4-(4-chlorobenzyl)-7-(methoxymethyl)-5,16-dimethyl-10-(3-(2-oxopyrrolidin-1-yl)propyl)hexadecahydrobenzo[l][1,4,7,11,14]penta-azacyclooctadecine-2,6,9,12,15(3H)-pentaone (Compound 296)

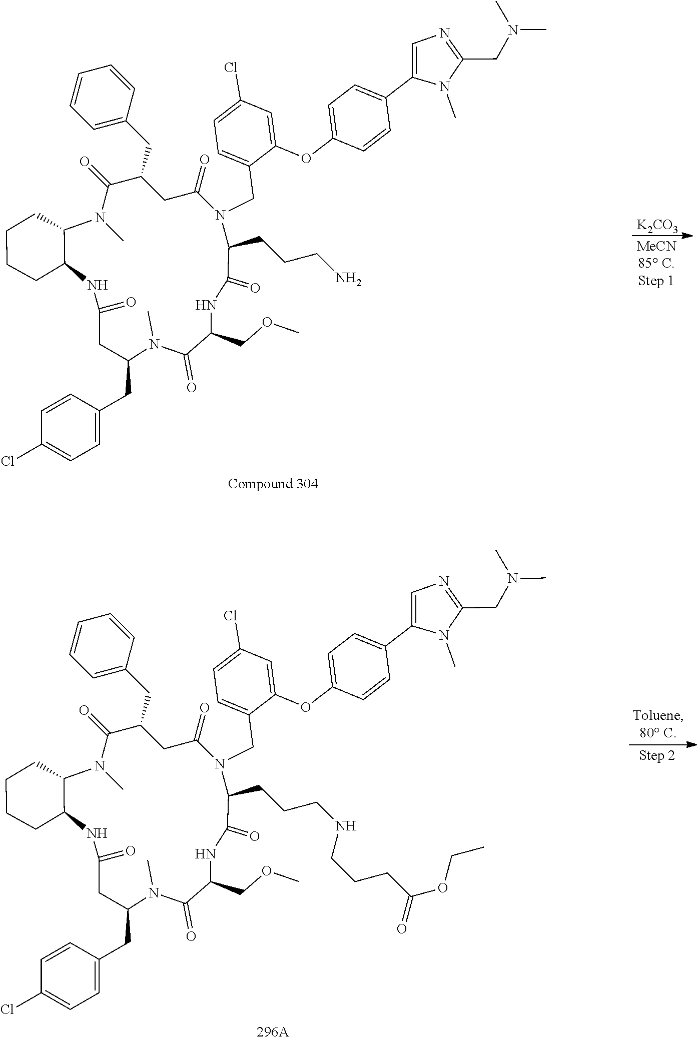 [SMILES: COC[C@@H]1NC(=O)[C@H](CCCN)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O ; "K2CO3" ; CCOC(=O)CCCNCCC[C@H]1C(=O)N[C@@H](COC)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1]

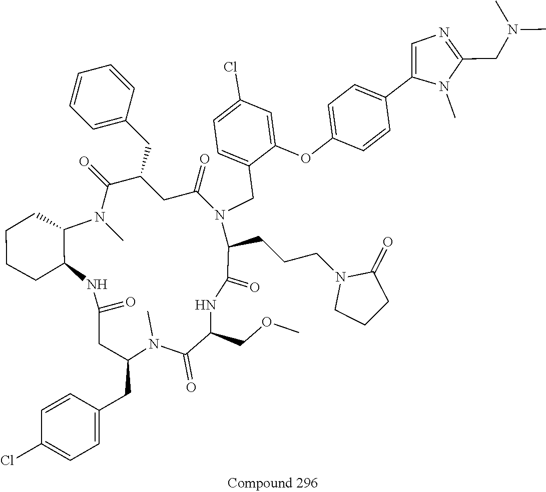 [SMILES: COC[C@@H]1NC(=O)[C@H](CCCN2CCCC2=O)N(Cc2ccc(Cl)cc2Oc2ccc(-c3cnc(CN(C)C)n3C)cc2)C(=O)C[C@@H](Cc2ccccc2)C(=O)N(C)[C@H]2CCCC[C@@H]2NC(=O)C[C@H](Cc2ccc(Cl)cc2)N(C)C1=O]

### Step 1. Ethyl 4-((3-((4S,7S,10S,14R,16aS,20aS)-14-benzyl-11-(4-chloro-2-(4-(2-((dimethylamino) methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)propyl)amino) butanoate (296A)

To a solution of Compound 304 (30 mg, 0.025 mmol) in 6 mL of anhydrous ACN was added K₂CO₃ (13.90 mg, 0.101 mmol), NaI (4.22 mg, 0.028 mmol), and ethyl-4-bromo butyrate (4.90 mg, 0.025 mmol) and the resulting mixture was heated to 85° C. overnight. The reaction mixture was then filtered through a syringe and directly purified via reversed-phase HPLC (eluting with 60→75% MeCN in water, 0.1% NH₄OH, 10 min gradient, XBridge Peptide BEH C18 5 um 19×150 mm). Clean fractions were collected and lyophilized to yield intermediate 296A (10 mg, 27%). Analytical method 7; t_{R}=1.30 min; [M+H]⁺=1193.7.

### Step 2. (4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(3-(2-oxopyrrolidin-1-yl)propyl)hexadecahydrobenzo[l][1,4,7,11,14]pentaazacyclooctadecine-2,6,9,12,15 (3H)-pentaone (Compound 296)

296A (10 mg, 8.38 μmol) was dissolved in toluene (6 mL) and the resulting mixture was stirred at 80° C. for 24 h. The reaction mixture was concentrated under reduced pressure and directly purified via reversed-phase HPLC (eluting with 60-75% MeCN in water, 0.1% NH₄OH, 10 min gradient, XBridge Peptide BEH C18 5 m 19×150 mm). Clean fractions were collected and lyophilized to yield Compound 296 (1.5 mg, 13.5%). Analytical method 3; t_{R}=1.08 min; [M+H]⁺=1145.53.

### Example 3.16: Synthesis of ((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-5,10,16-trimethyl-2,6,9,12,15-pentaoxodocosahydrobenzo[l][1,4,7,11,14]pentaazacyclooctadecin-7-yl)methyl pivalate (Compound 226)

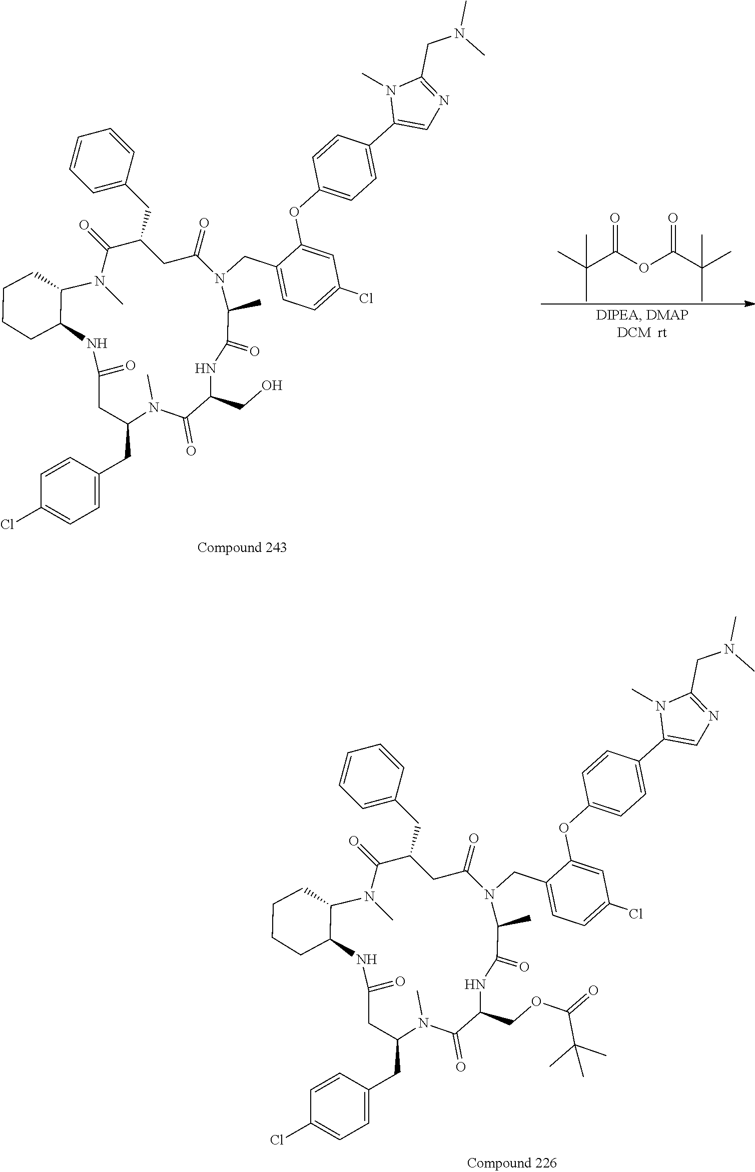 [SMILES: C[C@H]1C(=O)N[C@@H](CO)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1 ; CC(C)(C)C(=O)OC(=O)C(C)(C)C ; C[C@H]1C(=O)N[C@@H](COC(=O)C(C)(C)C)C(=O)N(C)[C@@H](Cc2ccc(Cl)cc2)CC(=O)N[C@H]2CCCC[C@@H]2N(C)C(=O)[C@H](Cc2ccccc2)CC(=O)N1Cc1ccc(Cl)cc1Oc1ccc(-c2cnc(CN(C)C)n2C)cc1]

To Compound 243 (72 mg, 0.070 mmol) in DCM (5 mL) at 0° C. was added DMAP (1.0 mg, 0.8 mmol), followed by DIPEA (0.024 mL, 0.140 mmol). After 5 min, trimethylacetic anhydride (105 mg, 0.56 mmol) in DCM (1 mL) was added and the resulting mixture was stirred at rt for 48 h. The reaction mixture was concentrated and purified via reversed-phase HPLC (0-100% MeCN in water with 0.1% NH₄OH) to afford white solid Compound 226 (44.0 mg, 56.7%). Analytical method 6; t_{R}=2.64 min; [M+H]⁺=1105.5.

### Example 3.17: Synthesis of (2S,5S,8S,13S,16R)-16-benzyl-1-(4-chloro-2-(4-(4-((diethylamino)methyl)-1H-1,2,3-triazol-1-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone (Compound 120)

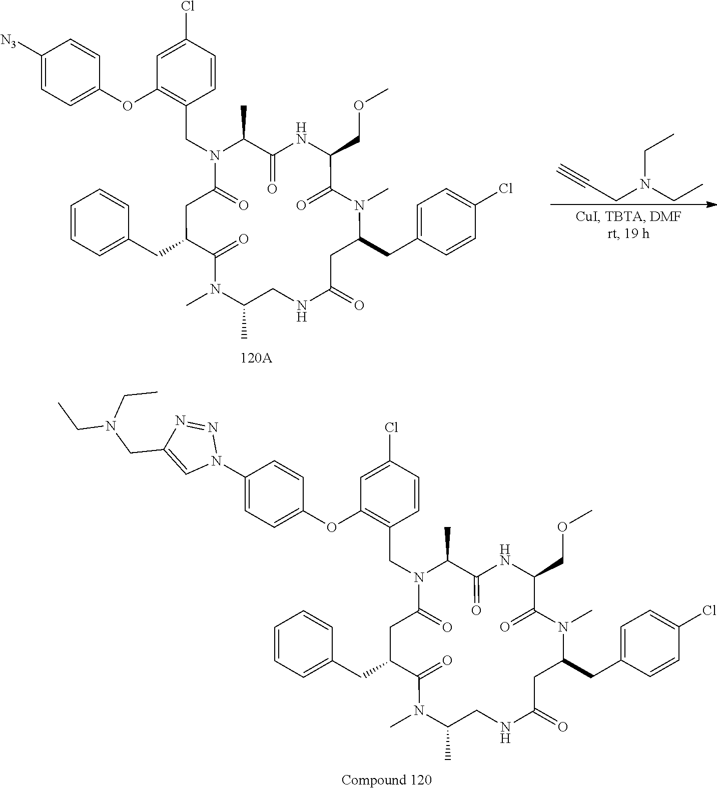

To Intermediate 120A (9.5 mg, 10.6 μmol) taken up in DMF (0.3 mL) in an Eppendorf cap was added N,N-diethylprop-2-yn-1-amine (3 μL, 0.022 mmol), a solution of tris[(1-benzyl-1H-1,2,3-triazol-4-yl)methyl]amine (TBTA, 0.019 M, 111 μL, 2.11 μmol) in DMF, and copper (I) iodide (2.6 mg, 0.014 mmol). The resulting mixture was put on a shaker for 19 h at rt and then loaded onto a Biotage Isolute Si-TMT column, eluting with MeOH. The filtrate was concentrated. The crude product was purified by reversed-phase HPLC (eluting with ACN/water with 0.1% TFA) and clean fractions were collected and lyophilized to yield Compound 120 (2.0 mg, 19%). Analytical method 4; t_{R}=2.20 min; [M+H]⁺=1010.9.

The Compounds shown in Table 15 below were prepared according to the procedure used for Compound 120 (Example 3.17).

TABLE 15

| Cmp | | Synthesis | |
|---|---|---|---|
| No. | Structure | Procedure | LCMS |
| 127 | 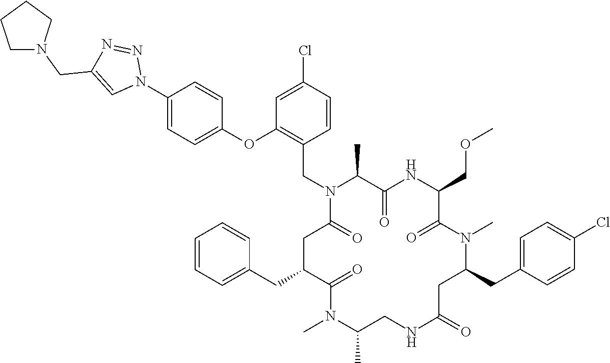 [SMILES: COC[C@@H]1NC(=O)[C@H](C)N(Cc2ccc(Cl)cc2Oc2ccc(-n3cc(CN4CCCC4)nn3)cc2)C(=O)C[C@@H](Cc2ccccc2)C(=O)N(C)[C@@H](C)CNC(=O)C[C@H](Cc2ccc(Cl)cc2)N(C)C1=O] | Compound 120 (Example 3.17) | Analytical method 4 t_{R} = 2.13 min [M + H]⁺ = 1008.6 |
| 128 | 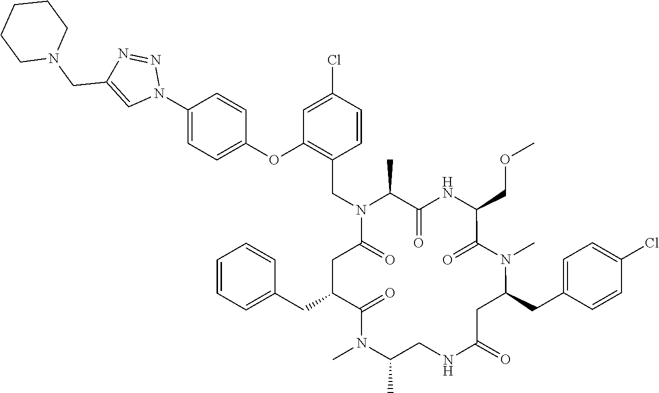 [SMILES: COC[C@@H]1NC(=O)[C@H](C)N(Cc2ccc(Cl)cc2Oc2ccc(-n3cc(CN4CCCCC4)nn3)cc2)C(=O)C[C@@H](Cc2ccccc2)C(=O)N(C)[C@@H](C)CNC(=O)C[C@H](Cc2ccc(Cl)cc2)N(C)C1=O] | Compound 120 (Example 3.17) | Analytical method 4 t_{R} = 2.20 min [M + H]⁺ = 1022.6 |

### Example 3.18: Synthesis of (2S,5S,8S,13S,16R)-16-benzyl-1-(4-chloro-2-(4-(4-((dimethylamino)methyl)-5-methyl-1H-1,2,3-triazol-1-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone formate (Compound 134)

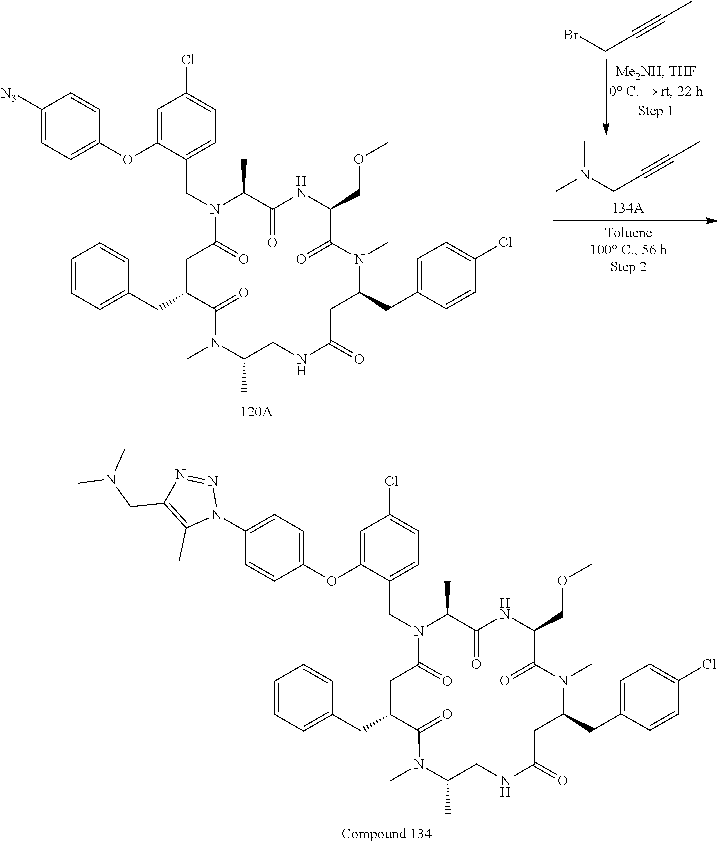

### Step 1. N,N-Dimethylbut-2-yn-1-amine (134A)

1-Bromo-2-butyne (0.3 mL, 3.32 mmol) was added dropwise to dimethylamine solution (2 M in THF, 4.5 mL, 9.00 mmol) at 0° C. The resulting mixture was allowed to come to rt over 22 h, then quenched with water, and partitioned with diethyl ether. The biphasic mixture was transferred to a separatory funnel. The layers were separated and the aqueous layer was extracted with ether (2×). The combined organic layers were dried with magnesium sulfate, filtered, and concentrated. The crude product (red oil) was dried under a stream of nitrogen, providing 134A (90 mg, 10%), which was carried onto to the next step without purification. ¹H NMR (400 MHz, CDCl₃) b 3.18-3.14 (m, 2H), 2.27 (s, 6H), 1.85-1.81 (m, 3H).

### Step 2. (2S,5S,8S,13S,16R)-16-Benzyl-1-(4-chloro-2-(4-(4-((dimethylamino)methyl)-5-methyl-1H-1,2,3-triazol-1-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone formate (Compound 134)

Intermediate 120A (20 mg, 0.022 mmol) and intermediate 134A (9 mg, 0.093 mmol) were taken up in toluene (0.1 mL) in a sealed vial and was heated to 100° C. Additional toluene (0.1 mL) was added after 5 h. LCMS indicated 13% of the product after 56 h, at which point the mixture was diluted with DMSO then purified via reversed-phase HPLC (eluting with ACN/water with 0.1% formic acid). The clean fractions were collected and lyophilized to yield Compound 134 (1.3 mg, 4%). Analytical method 4; t_{R}=2.83 min; [M+2H]²⁺=499.8.

### Example 3.19: Synthesis of (2S,5S,8S,13S,16R)-5-((1H-tetrazol-5-yl)methyl)-16-benzyl-1-(4-chloro-2-(4-(2-((dimethylamino) methyl)-1-methyl-1H-imidazol-5-yl)phenoxy)benzyl)-8-(4-chlorobenzyl)-2,7,13,14-tetramethyl-1,4,7,11,14-pentaazacyclooctadecane-3,6,10,15,18-pentaone (Compound 148)

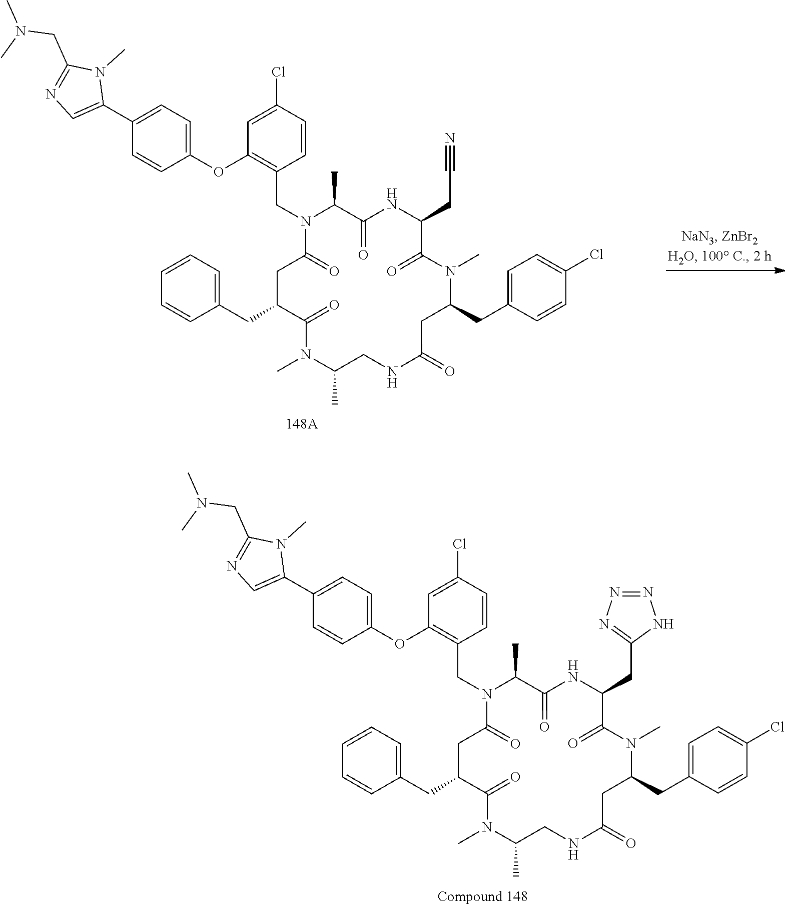 [SMILES: C[C@H]1CNC(=O)C[C@H](Cc2ccc(Cl)cc2)N(C)C(=O)[C@H](CC#N)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C ; C[C@H]1CNC(=O)C[C@H](Cc2ccc(Cl)cc2)N(C)C(=O)[C@H](Cc2nnn[nH]2)NC(=O)[C@H](C)N(Cc2ccc(Cl)cc2Oc2ccc(-c3cnc(CN(C)C)n3C)cc2)C(=O)C[C@@H](Cc2ccccc2)C(=O)N1C]

To a microwave vial charged with 148A (0.017 g, 0.017 mmol), ZnBr₂ (3.86 mg, 0.017 mmol), and NaN₃ (1.227 mg, 0.019 mmol) was added water (0.5 mL) and the resulting mixture was heated in the microwave for 2 h at 100° C. The crude reaction mixture was filtered. The resulting solid was dissolved in MeOH/DMSO and purified by reversed-phase chromatography (C18 aq column eluting with ACN/water, 0.1% NH₄OH) affording Compound 148 (0.4 mg, 0.348 μmol, 2% yield) as a white solid after lyophilization. Analytical method 4; t_{R}=1.97 min; [M+H]⁺=1033.4.

### Example 3.20: Synthesis of (2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-16-((5-fluoropyridin-2-yl)methyl)-5-(methoxymethyl)-2,7,13,14-tetramethyl-1,4,7,11,14-pentaazacyclooctadecane-3,6,10,15,18-pentaone (Compound 45)

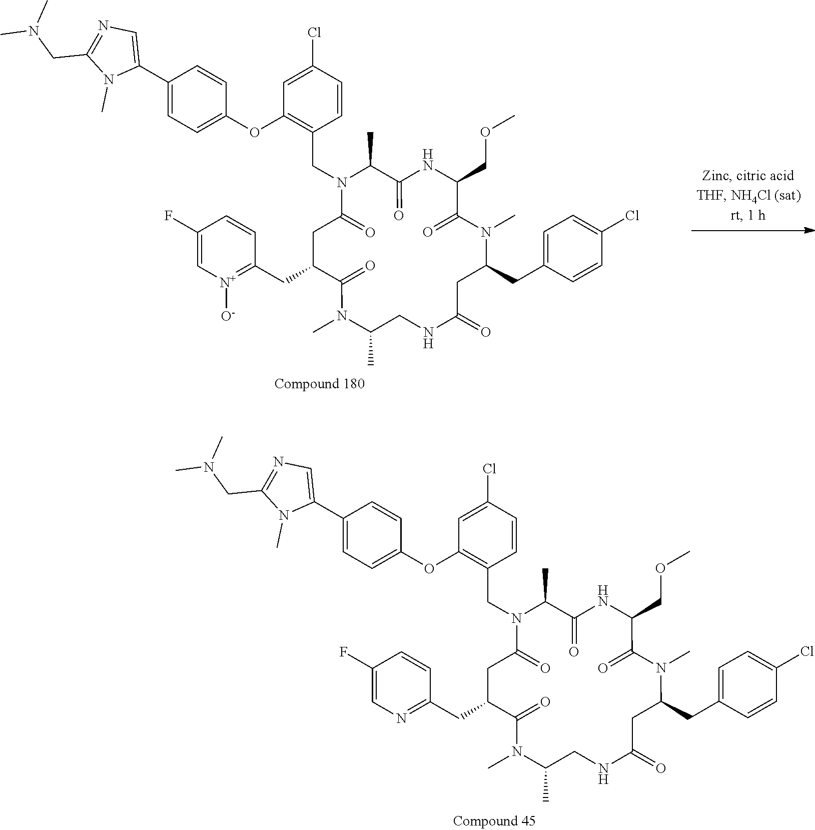 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccc(F)c[n+]2[O-])C(=O)N(C)[C@@H](C)CNC(=O)C[C@H](Cc2ccc(Cl)cc2)N(C)C1=O ; COC[C@@H]1NC(=O)[C@H](C)N(Cc2ccc(Cl)cc2Oc2ccc(-c3cnc(CN(C)C)n3C)cc2)C(=O)C[C@@H](Cc2ccc(F)cn2)C(=O)N(C)[C@@H](C)CNC(=O)C[C@H](Cc2ccc(Cl)cc2)N(C)C1=O]

To a solution of Compound 180 (30 mg, 0.029 mmol) in THF (3 mL) was added NH₄Cl (sat) (1 mL), zinc (80 mg, 1.224 mmol) and citric acid (100 mg, 0.520 mmol). The reaction mixture was stirred at rt for 1 h and then diluted with DCM. The pH of the solution was adjusted to pH 9-10 with NaHCO₃ (sat). The organic phase was dried over Na₂SO₄, filtered, and concentrated. The residue was purified by preparative reversed-phase HPLC using basic conditions (0-100% MeCN in water with 0.1% NH₄OH) to give Compound 45 as white solid (5.9 mg, 19.0%). Analytical method 4; t_{R}=1.85 min; [M+H]⁺=1014.7.

The Compounds shown in Table 16 below were prepared according to the procedure used for Compound 45 (Example 3.20).

TABLE 16

| Cmp | | Synthesis | |
|---|---|---|---|
| No. | Structure | Procedure | LCMS |
| 105 | 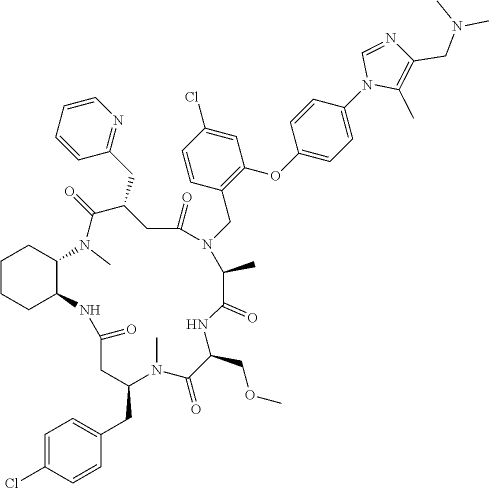 [SMILES: COC[C@@H]1NC(=O)[C@H](C)N(Cc2ccc(Cl)cc2Oc2ccc(-n3cnc(CN(C)C)c3C)cc2)C(=O)C[C@@H](Cc2ccccn2)C(=O)N(C)[C@H]2CCCC[C@@H]2NC(=O)C[C@H](Cc2ccc(Cl)cc2)N(C)C1=O] | Compound 45 (Example 3.20)- starting from intermediate 105A (see Example 2.8). | Analytical method 4 t_{R} = 1.68 min [M + H]⁺ = 1036.6 |
| 250 | 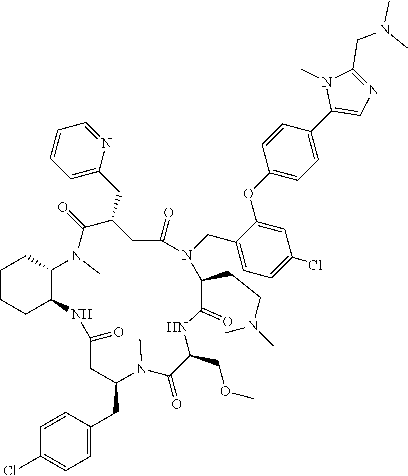 [SMILES: COC[C@@H]1NC(=O)[C@H](CCN(C)C)N(Cc2ccc(Cl)cc2Oc2ccc(-c3cnc(CN(C)C)n3C)cc2)C(=O)C[C@@H](Cc2ccccn2)C(=O)N(C)[C@H]2CCCC[C@@H]2NC(=O)C[C@H](Cc2ccc(Cl)cc2)N(C)C1=O] | Compound 45 (Example 3.20)- starting from intermediate 250I (see Example 2.10). | Analytical method 3 t_{R} = 0.80 min [M + H]⁺ = 1093.5 |
| 284 | 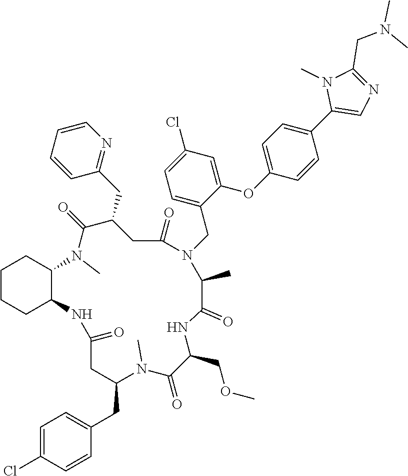 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccn2)C(=O)N(C)[C@H]2CCCC[C@@H]2NC(=O)C[C@H](Cc2ccc(Cl)cc2)N(C)C1=O] | Compound 45 (Example 3.20)- starting from Compound 172 (see Example 2.7). | Analytical method 4 t_{R} = 1.77 min [M + H]⁺ = 1038.7 |
| 295 | 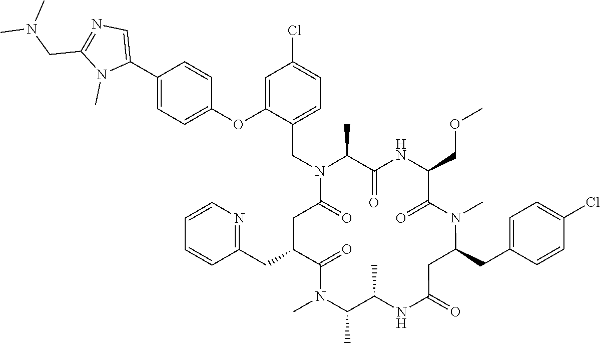 [SMILES: COC[C@@H]1NC(=O)[C@H](C)N(Cc2ccc(Cl)cc2Oc2ccc(-c3cnc(CN(C)C)n3C)cc2)C(=O)C[C@@H](Cc2ccccn2)C(=O)N(C)[C@@H](C)[C@H](C)NC(=O)C[C@H](Cc2ccc(Cl)cc2)N(C)C1=O] | Compound 45 (Example 3.20)- starting from Compound 180 (see Example 2.7). | Analytical method 4 t_{R} = 1.69 min [M + H]⁺ = 1010.7 |

### Example 3.21: Synthesis of tert-butyl 2-((25,55,85,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-3,6,10,15,18-pentaoxo-1,4,7,11,14-pentaazacyclooctadecan-5-yl)acetate (Compound 185)

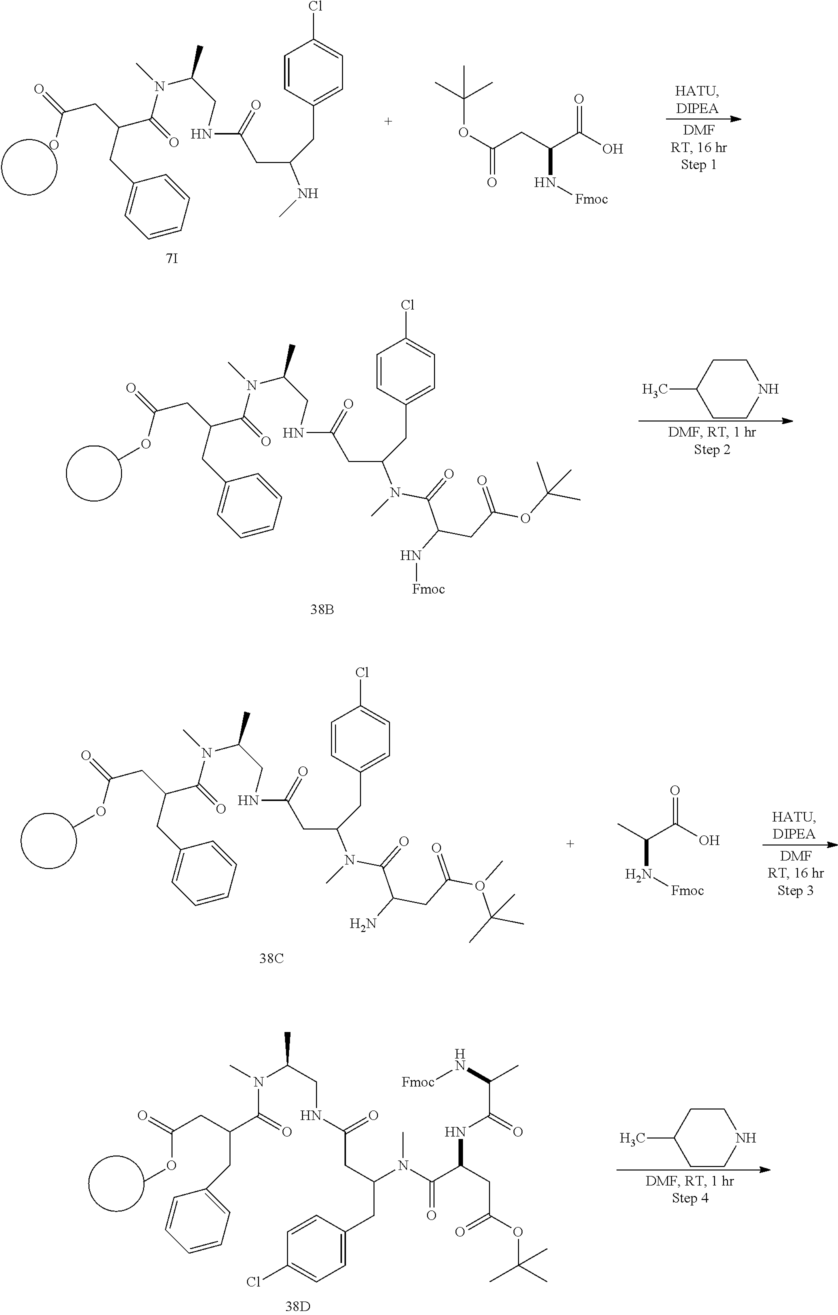

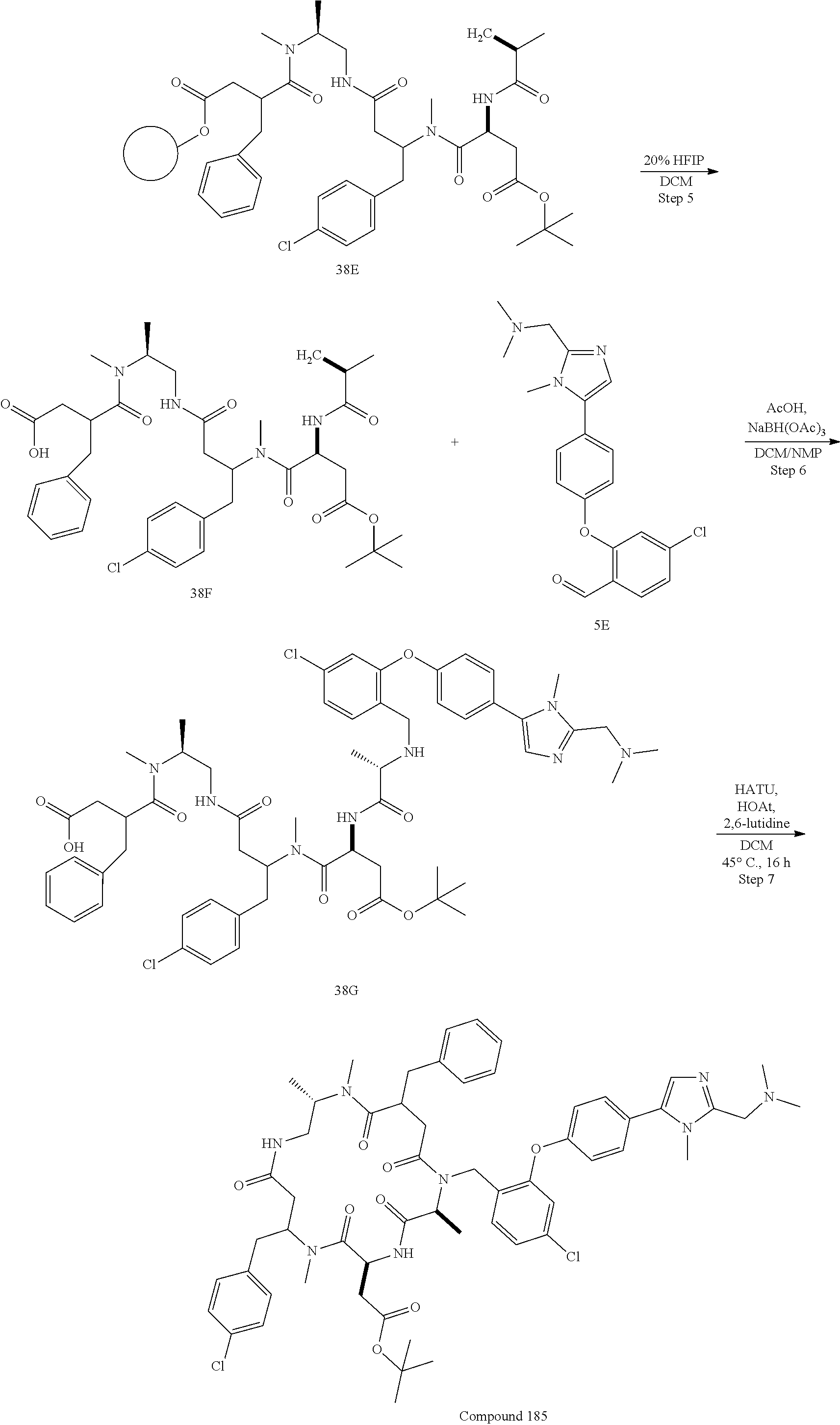

### Step 1. 2-chloro-trityl Resin Bound—5S,8S,13S,16R)-16-benzyl-5-(2-(tert-butoxy)-2-oxoethyl)-8-(4-chlorobenzyl)-1-(9H-fluoren-9-yl)-7,13,14-trimethyl-3,6,10,15-tetraoxo-2-oxa-4,7,11,14-tetraazaoctadecan-18-oic acid (38B)

To 7I resin (0.779 mg, 1.0 mmol) in DMF (10 mL) was added a pre-mixed solution of Fmoc-L-aspartic acid beta-t-butyl ester (0.823 g, 2.0 mmol), DIPEA (0.7 mL, 4.0 mmol) and HATU (0.760 g, 2.0 mmol) and the resulting mixture was shaken for 18 h at room temperature. The resin was filtered, washed with DMF (2×20 mL) and DCM (2×20 mL) and dried under vacuum. Resin 38B (1.75 g, crude) was taken onto the next step without purification.

### Step 2. 2-Chloro trityl Resin Bound—(6S,9S,14S,17R)-6-amino-17-benzyl-9-(4-chlorobenzyl)-2,2,8,14,15-pentamethyl-4,7,11,16-tetraoxo-3-oxa-8,12,15-triazanonadecan-19-oic acid (38C)

To 38B (1.75 g, 1.0 mmol) was added 20% 4-methylpiperidine in DMF (10 mL) and the resulting mixture was shaken at room temperature for 2 h. The resin was then filtered, washed with DMF (2×10 mL) and DCM (2×10 mL) and dried under vacuum. Resin 38C (1.75 g, crude) was taken onto the next step without purification.

### Step 3. 2-Chloro trityl Resin Bound—(5S,8S,11S,16S,19R)-19-benzyl-8-(2-(tert-butoxy)-2-oxoethyl)-11-(4-chlorobenzyl)-1-(9H-fluoren-9-yl)-5,10,16,17-tetramethyl-3,6,9,13,18-pentaoxo-2-oxa-4,7,10,14,17-pentaazahenicosan-21-oic acid (38D)

To 38C (1.75 g, 1.0 mmol) in DMF (10 mL) was added a pre-mixed solution of Fmoc-alanine-OH (0.623 g, 2.0 mmol), DIPEA (0.699 mL, 4.0 mmol) and HATU (0.760 g, 2.0 mmol) and the resulting mixture was shaken for 18 h at room temperature. The resin was then filtered, washed with DMF (2×20 mL) and DCM (2×20 mL), and dried under vacuum. Resin 38D (1.75 g, crude) was taken onto the next step without purification.

### Step 4. 2-Chloro trityl Resin Bound—(6S,9S,14S,17R)-6-((S)-2-aminopropanamido)-17-benzyl-9-(4-chloro benzyl)-2,2,8,14,15-pentamethyl-4,7,11,16-tetraoxo-3-oxa-8,12,15-triazanonadecan-19-oic acid (38E)

To 38D (1.75 g, 1.0 mmol) was added 20% 4-methylpiperidine in DMF (10 mL) and the resulting mixture was shaken at room temperature for 2 h. The resin was then filtered, washed with DMF (2×10 mL) and DCM (2×10 mL), and dried under vacuum. Resin 38E (1.75 g, crude) was taken onto the next step without purification.

### Step 5. (6S,9S,14S,17R)-6-((S)-2-aminopropanamido)-17-benzyl-9-(4-chloro benzyl)-2,2,8,14,15-pentamethyl-4,7,11,16-tetraoxo-3-oxa-8,12,15-triazanonadecan-19-oic acid (38F)

38E (1.75 g, 1.0 mmol) was cleaved by shaking with 20 mL of 20% HFIP/DCM for 20 min at room temperature in a syringe filter. The resin was filtered and the filtrate was collected. Both steps were repeated three more times to ensure all of the product was cleaved from the resin. The combined filtrates were concentrated to provide a crude oil which was purified via reverse phase chromatography (eluting with 0-100% MeCN/water with 0.1% NH₄OH) to afford 38F as a white solid (526 mg, 74%). Analytical method 1; t_{R}=1.14 min; [M+H]⁺=730.4.

### Step 6. (3S,6S,9S,14S,17R)-17-benzyl-6-(2-(tert-butoxy)-2-oxoethyl)-1-(4-chloro-2-(4-(2-((dimethyl amino)methyl)-1-methyl-1H-imidazol-5-yl)phenoxy)phenyl)-9-(4-chlorobenzyl)-3,8,14,15-tetramethyl-4,7,11,16-tetraoxo-2,5,8,12,15-pentaazanonadecan-19-oic acid (38G)

To intermediate 5E (185 mg, 0.5 mmol) in a vial was added a solution of 38F (366 mg, 0.5 mmol) in DCM (16 mL) followed by AcOH (0.086 mL, 1.5 mmol) and the resulting mixture was stirred at room temperature overnight. The reaction mixture was then concentrated to dryness and MeOH (8 mL) was added. This mixture was cooled to 0° C., NaBH₄ (95 mg, 2.51 mmol) was added in one portion. The resulting mixture was stirred in the ice bath for 30 min and then quenched with water (0.5 mL) and acetic acid (0.086 mL, 1.504 mmol), allowed to warm to room temperature, and directly purified by reverse phase chromatography using C18 column (eluting with 0-100% ACN/water with 0.1% TFA gradient). This provided 38G as a white solid (576 mg, 96%). Analytical method 1; t_{R}=1.12 min; [M+H]⁺=1084.1.

### Step 7. tert-butyl 2-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-3,6,10,15,18-pentaoxo-1,4,7,11,14-pentaazacyclooctadecan-5-yl)acetate (Compound 185)

To a mixture of intermediate 38G (576 mg, 0.48 mmol) in DCM (260 mL) at room temperature was added HOAt (164 mg, 1.20 mmol) and HATU (457 mg, 1.20 mmol), followed by 2,6-lutidine (0.840 mL, 7.21 mmol) and the resulting mixture was heated to 45° C. overnight. The reaction mixture was then concentrated to afford a crude oil, which was purified directly by reverse phase column chromatography using C18 column (eluting with 0-100% ACN/water 0.1% NH₄OH). This provided Compound 185 (233 mg, 46%) as a white solid. Analytical method 6; t_{R}=2.37 min; [M+H]⁺=1065.48.

### Example 4: Building Block A (BB A)—Succinates

### Example 4.1: Synthesis of (R)-2-benzyl-4-(tert-butoxy)-4-oxobutanoic acid (A1)

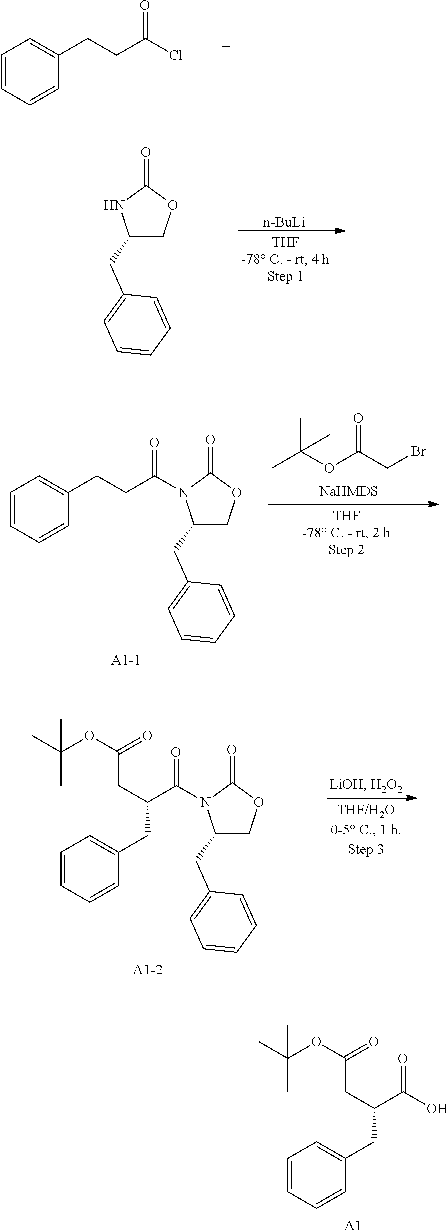

### Step 1. (S)-4-benzyl-3-(3-phenylpropanoyl)oxazolidin-2-one (A1-1)

To a cold stirred solution of (S)-4-benzyloxazolidin-2-one (500 g, 2.821 mol) in THF (9 L) was added n-BuLi (2.5M in hexane) (1.24 L, 3.103 mol) slowly over a period of 30 minutes at −78° C. and the resulting mixture was stirred for 30 minutes at −78° C. A solution of 3-phenylpropanoyl chloride (571 g, 3.38 mol) in THF (1 L) was then slowly added over a period of 1 h at −78 to −60° C. and the reaction mixture was slowly warmed to room temperature for 2 h. The reaction mixture was cooled to 0° C., quenched with sat. NH₄Cl (500 mL) and extracted with dichloromethane (2×1.5 L). The combined organic layers were washed with 0.5 N NaOH (1 L) and brine (1 L), dried over anhydrous sodium sulfate, filtered, and evaporated under reduced pressure. Crude A1-1 was triturated with petroleum ether (5 L) for 1 h. The solid product was filtered, washed with petroleum ether (500 mL), and dried under vacuum to afford compound A1-1 (815 g, 93%) as an off-white solid. Analytical method 7; t_{R}=1.53 min; [M+H]⁺=310.2.

### Step 2. (R)-tert-butyl 3-benzyl-4-((S)-4-benzyl-2-oxooxazolidin-3-yl)-4-oxobutanoate (A1-2)

To a cold stirred solution of A1-1 (500 g, 1.616 mol) in THF (7 L), was added 1.0 M NaHMDS in THF (1.94 L, 1.939 mol) slowly over a period of 30 minutes at −78° C. The resulting mixture was stirred for 1 h at −78° C. and a solution of tert-butyl-2-bromo acetate (472.8 g, 2.424 mol) in THF (500 mL) was then added drop wise over a period of 30 min at −78° C. The mixture was stirred for 2 h, then quenched with sat. NH₄Cl (500 mL), and extracted with ethyl acetate (2×1.5 L). The combined organic layers were washed with brine solution (2 L), dried over anhydrous sodium sulfate, filtered, and concentrated under reduced pressure. The crude material was triturated with methanol (800 mL) for 1 h, after which the solid product was filtered, washed with methanol (200 mL) and dried under vacuum to afford compound A1-2 (410 g, 60%) as an off-white solid. Analytical method 7; t_{R}=1.78 min; [M-tBu]⁺=368.3.

### Step 3. (R)-2-benzyl-4-(tert-butoxy)-4-oxobutanoic acid (A1)

To a cold stirred solution of A1-2 (250 g, 0.59 mol) in THF (9 L) was added 30% H₂O₂ (267 mL, 2.37 mol) at 0-5° C. and the reaction was stirred for 30 min at same temperature. Then a solution of LiOH.H₂O (49.5 g, 1.18 mol) in water (3 L) was added to the above reaction mixture at 0-5° C. and the mixture was stirred for 1 h. The reaction mixture was quenched with sat. sodium sulfite (1.6 L) and sat. sodium bicarbonate (1.6 L). Then solvent was concentrated under reduced pressure, diluted with water (3 L) and washed with DCM (2×1 L) to remove the impurities. Then the aqueous layer was cooled to 5° C. and acidified to pH ˜1.5 with 6 M HCl (1 L). Product was extracted with ethyl acetate (3×1 L). The combined organic layer was washed with brine solution (1 L), dried over anhydrous sodium sulfate, and concentrated under vacuum to afford compound A1 as an oil (125 g, 82%). Analytical method 7; t_{R}=1.78 min; [M−H]⁻=263.5. ¹H NMR (400 MHz, Chloroform-d) δ 1.42 (s, 9H), 2.36 (dd, J=16.93, 4.58 Hz, 1H), 2.48-2.62 (m, 1H), 2.71-2.83 (m, 1H), 3.00-3.18 (m, 2H), 7.12-7.35 (m, 6H).

The succinate building blocks shown in Table 17 below were prepared according to the procedure used for A1 (Example 4.1), A8 (Example 4.4), A8-3 (Example 4.4, Steps 1-3), A26 (Example 4.5).

TABLE 17
Succinates-Builind block A

| BB | | | Synthesis procedure |
|---|---|---|---|
| No. | Structure | Name | LCMS/NMR |
| A2 | 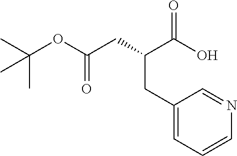 [SMILES: CC(C)(C)OC(=O)C[C@@H](Cc1cccnc1)C(=O)O] | (R)-4-(tert-butoxy)- 4-oxo-2-(pyridin-3- ylmethyl)butanoic acid | A1 (Example 4.1)- starting from intermediate A2-1 (see Example 4.2)/Analytical method 10 t_{R} = 0.61 min [M + H]⁺ = 266.1 |
| A3 | 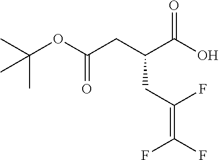 [SMILES: CC(C)(C)OC(=O)C[C@@H](CC(F)=C(F)F)C(=O)O] | (S)-2-(2-(tert- butoxy)-2- oxoethyl)-4,5,5- trifluoropent-4- enoic acid | A1 (Example 4.1)- starting from intermediate A3-1 (see Table 18)/ Analytical method 7 t_{R} = 0.93 min [M − H]⁻ = 267.2 |
| A4 | 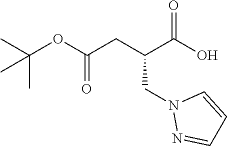 [SMILES: CC(C)(C)OC(=O)C[C@@H](Cn1cccn1)C(=O)O] | (S)-2-((1H-pyrazol- 1-yl)methyl)-4- (tert-butoxy)-4- oxobutanoic acid | A1 (Example 4.1)- starting from intermediate A4-3 (see Table 18)/ Analytical method 5 t_{R} = 0.46 min [M − H]⁻ = 253.1 |
| A5 | 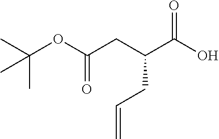 | (R)-2-(2-(tert- butoxy)-2- oxoethyl)pent-4- enoic acid | A1 (Example 4.1)- starting from intermediate A5-1 (see Table 18)/ Analytical method 7 t_{R} = 0.83 min [M − H]⁻ = 213.4 |
| A6 | 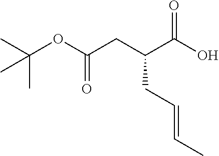 [SMILES: C/C=C/C[C@H](CC(=O)OC(C)(C)C)C(=O)O] | ((R,E)-2-(2-(tert- butoxy)-2- oxoethyl)hex-4- enoic acid | A8-3 (Example 4.4, Steps 1-3)- starting from A8-1 and (E)-1- bromobut-2-ene./ Analytical method 4 t_{R} = 2.03 min [M − tBu]⁺ = 173.3 |
| A7 | 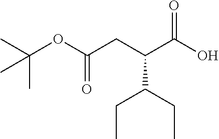 [SMILES: CCC(CC)[C@H](CC(=O)OC(C)(C)C)C(=O)O] | (S)-2-(2-(tert- butoxy)-2- oxoethyl)-3- ethylpentanoic acid | A1 (Example 4.1)- starting from intermediate A7-1 (see Table 18)/ Analytical method 5 t_{R} = 0.52 min [M − H]⁻ = 243.1 |
| A9 | 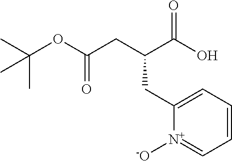 | (R)-2-(4-(tert- butoxy)-2-carboxy- 4- oxobutyl)pyridine 1-oxide | A8 (Example 4.4)- starting from A8-1 and the corresponding bromide/Analytical method 7 t_{R} = 0.60 min [M + H]⁺ = 280.3 |
| A10 | 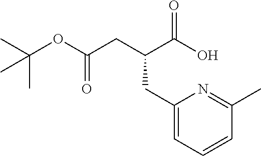 [SMILES: Cc1cccc(C[C@H](CC(=O)OC(C)(C)C)C(=O)O)n1] | (R)-4-(tert-butoxy)- 2-((6- methylpyridin-2- yl)methyl)-4- oxobutanoic acid | A8-3 (Example 4.4, Steps 1-3)- starting from A8-1 and the corresponding bromide/ Analytical method 7 t_{R} = 0.56 min [M + H]⁺ = 281.3 |
| A11 | 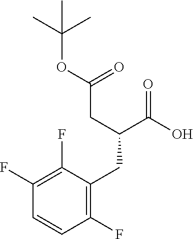 [SMILES: CC(C)(C)OC(=O)C[C@@H](Cc1c(F)ccc(F)c1F)C(=O)O] | (R)-4-(tert-butoxy)- 4-oxo-2-(2,3,6- tritluorobenzyl)- butanoic acid | A8-3 (Example 4.4, Steps 1-3)- starting from A8-1 and the corresponding bromide/ Analytical method 7 t_{R} = 1.02 min [M − H]⁻ = 317.0 |
| A12 | 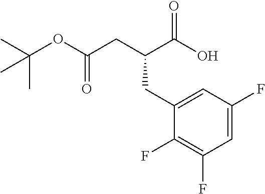 [SMILES: CC(C)(C)OC(=O)C[C@@H](Cc1cc(F)cc(F)c1F)C(=O)O] | (R)-4-(tert-butoxy)- 4-oxo-2-(2,3,5- trifluorobenzyl)- butanoic acid | A8-3 (Example 4.4, Steps 1-3)- starting from A8-1 and the corresponding bromide/ Analytical method 7 t_{R} = 1.04 min [M − H]⁻ = 317.0 |
| A13 | 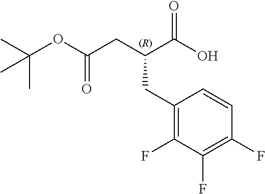 | (R)-4-(tert-butoxy)- 4-oxo-2-(2,3,4- tnfluorobenzyl)- butanoic acid | A8-3 (Example 4.4, Steps 1-3)- starting from A8-1 and the corresponding bromide/ Analytical method 5 t_{R} = 0.61 min [M − H]⁻ = 317.0 |
| A14 | 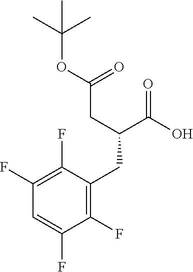 [SMILES: CC(C)(C)OC(=O)C[C@@H](Cc1c(F)c(F)cc(F)c1F)C(=O)O] | (R)-4-(tert-butoxy)- 4-oxo-2-(2,3,5,6- tetrafluorobenzyl)- butanoic acid | A8 (Example 4.4)/ ¹H NMR (400 MHz, DMSO-d₆) δ 12.53 (s, 1H), 7.79 (ddd, J = 10.6, 7.7, 2.9 Hz, 1H), 3.05 (d, J = 6.3 Hz, 1H), 2.97-2.81 (m, 2H), 2.45 (dd, J = 8.4, 6.5 Hz, 2H), 1.36 (s, 9H). |
| A15 | 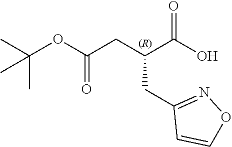 | (R)-4-(tert-butoxy)- 2-(isoxazol-3- ylmethyl)-4- oxobutanoic acid | A8-3 (Example 4.4, Steps 1-3)- starting from A8-1 and the corresponding bromide/ ¹H NMR (400 MHz, Chloroform- d) δ 8.25 (s, 1H), 6.27 (d, J = 1.3 Hz, 1H), 3.04 (dd, J = 15.9, 8.1 Hz, 1H), 2.98-2.79 (m, 2H), 2.75- 2.53 (m, 2H), 1.40 (s, 9H). |
| A16 | 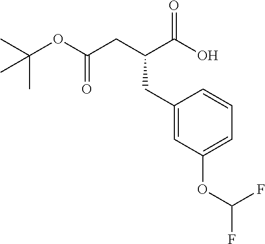 [SMILES: CC(C)(C)OC(=O)C[C@@H](Cc1cccc(OC(F)F)c1)C(=O)O] | (R)-4-(tert-butoxy)- 2-(3- (difluoromethoxy)- benzyl)-4- oxobutanoic acid | A8-3 (Example 4.4, Steps 1-3)- starting from A8-1 and the corresponding bromide/ Analytical method 7 t_{R} = 1.03 min [M − H]⁻ = 329.3 |
| A17 | 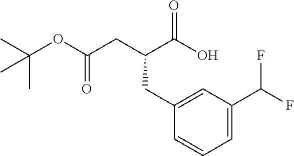 [SMILES: CC(C)(C)OC(=O)C[C@@H](Cc1cccc(C(F)F)c1)C(=O)O] | (R)-4-(tert-butoxy)- 2-(3- (difluoromethyl)- benzyl)-4- oxobutanoic acid | A8-3 (Example 4.4, Steps 1-3)- starting from A8-1 and the corresponding bromide/ Analytical method 7 t_{R} = 1.01 min [M − H]⁻ = 313.3 |
| A18 | 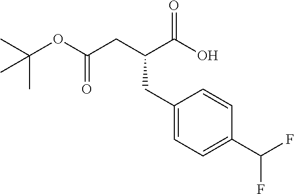 [SMILES: CC(C)(C)OC(=O)C[C@@H](Cc1ccc(C(F)F)cc1)C(=O)O] | (R)-4-(tert-butoxy)- 2-(4- (difluoromethyl)- benzyl)-4- oxobutanoic acid | A8-3 (Example 4.4, Steps 1-3)- starting from A8-1 and the corresponding bromide/ Analytical method 2 t_{R} = 0.97 min [M − H]⁻ = 313.3 |
| A19 | 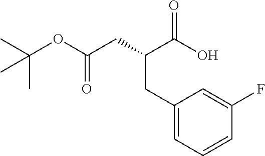 [SMILES: CC(C)(C)OC(=O)C[C@@H](Cc1cccc(F)c1)C(=O)O] | (R)-4-(tert-butoxy)- 2-(3-fluorobenzyl)- 4-oxobutanoic acid | A8-3 (Example 4.4, Steps 1-3)- starting from A8-1 and the corresponding bromide/ Analytical method 10 t_{R} = 1.03 min [M − H]⁻ = 281.2 |
| A20 | 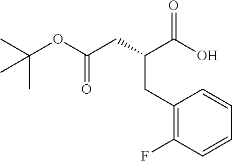 [SMILES: CC(C)(C)OC(=O)C[C@@H](Cc1ccccc1F)C(=O)O] | (R)-4-(tert-butoxy)- 2-(2-fluorobenzyl)- 4-oxobutanoic acid | A8-3 (Example 4.4, Steps 1-3)- starting from A8-1 and the corresponding bromide/ Analytical method 10 t_{R} = 1.02 min [M − H]⁻ = 281.2 |
| A21 | 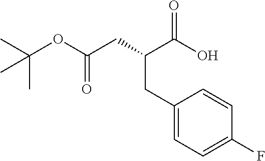 [SMILES: CC(C)(C)OC(=O)C[C@@H](Cc1ccc(F)cc1)C(=O)O] | (R)-4-(tert-butoxy)- 2-(4-fluorobenzyl)- 4-oxobutanoic acid | A8-3 (Example 4.4, Steps 1-3)- starting from A8-1 and the corresponding bromide/ Analytical method 10 t_{R} = 1.03 min [M − H]⁻ = 281.2 |
| A22 | 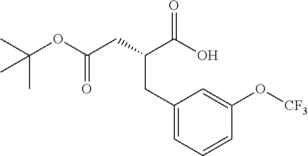 [SMILES: CC(C)(C)OC(=O)C[C@@H](Cc1cccc(OC(F)(F)F)c1)C(=O)O] | (R)-4-(tert-butoxy)- 4-oxo-2-(3- (trifluoromethoxy)- benzyl)butanoic acid | A8-3 (Example 4.4, Steps 1-3)- starting from A8-1 and the corresponding bromide/ Analytical method 10 t_{R} = 1.15 min [M − H]⁻ = 347.3 |
| A23 | 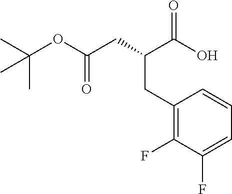 [SMILES: CC(C)(C)OC(=O)C[C@@H](Cc1cccc(F)c1F)C(=O)O] | (R)-4-(tert-butoxyl- 2-(2,3- difluorobenzyl)-4- oxobutanoic acid | A8-3 (Example 4.4, Steps 1-3)- starting from A8-1 and the corresponding bromide/ Analytical method 10 t_{R} = 1.05 min [M − H]⁻ = 299.2 |
| A24 | 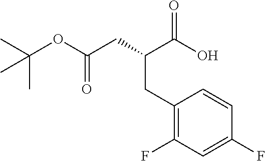 [SMILES: CC(C)(C)OC(=O)C[C@@H](Cc1ccc(F)cc1F)C(=O)O] | (R)-4-(tert-butoxy)- 2-(2,4- difluorobenzyl)-4- oxobutanoic acid | A8-3 (Example 4.4, Steps 1-3)- starting from A8-1 and the corresponding bromide/ Analytical method 10 t_{R} = 1.04 min [M − H]⁻ = 299.2 |
| A25 | 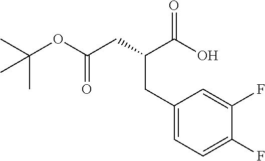 [SMILES: CC(C)(C)OC(=O)C[C@@H](Cc1ccc(F)c(F)c1)C(=O)O] | (R)-4-(tert-butoxy)- 2-(3,4- ditluorobenzyl)-4- oxobutanoic acid | A8-3 (Example 4.4, Steps 1-3)- starting from A8-1 and the corresponding bromide/ Analytical method 10 t_{R} = 1.06 min [M − H]⁻ = 299.2 |
| A26 | 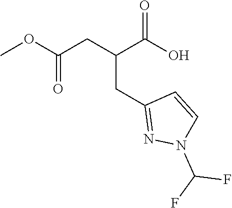 [SMILES: COC(=O)CC(Cc1ccn(C(F)F)n1)C(=O)O] | 2-((1- (difluoromethyl)- 1H-pyrazol-3- yl)methyl)-4- methoxy-4- oxobutanoic acid (racemic) | A26 (Example 4.5)/ Analytical method 10 t_{R} = 0.61 min [M + H]⁺ = 263.1 |
| A29 | 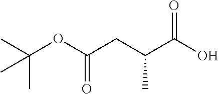 | (R)-4-(tert-butoxy)- 2-methyl-4- oxobutanoic acid | Commercially available |
| A30 | 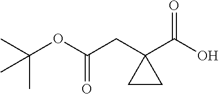 | 1-(2-methoxy-2- oxoethyl)- cyclopropane- 1-carboxylic acid | A1 (Example 4.1)/ ¹H NMR (400 MHz, Chloroform- d) δ 3.70 (s, 3H), 2.54 (s, 2H), 1.44 (q, J = 4 4 Hz, 2H), 0.90 (q, J = 4.4 Hz, 2H). |
| A31 | 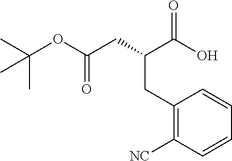 [SMILES: CC(C)(C)OC(=O)C[C@@H](Cc1ccccc1C#N)C(=O)O] | (R)-4-(tert-butoxy)- 2-(2-cyanobenzyl)- 4-oxobutanoic acid | A8-3 (Example 4.4, Steps 1-3)- starting from A8-1 and the corresponding bromide/ Analytical method 7 t_{R} = 0.90 min [M − H]⁻ = 288.2 |
| A32 | 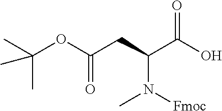 | (S)-2-((((9H- fluoren-9- yl)methoxy)- carbonyl)- (methyl)amino)- 4-(tert-butoxy)-4- oxobutanoic acid | commercially available |
| A33 | 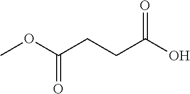 | 4-methoxy-4- oxobutanoic acid | commercially available |
| A34 | 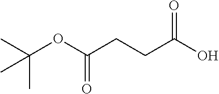 | 4-(tert-butoxy)-4- oxobutanoic acid | commercially available |
| A35 | 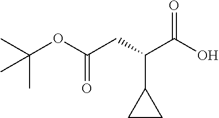 | (S)-4-(tert-butoxy)- 2-cyclopropyl-4- oxobutanoic acid | A1 (Example 4.1)/¹H NMR (400 MHz, DMSO-d₆): δ ppm: 12.14 (s, 1H), 2.57-2.23 (m, 1H), 2.42-2.37 (dd, J = 5.0 Hz & 16.1 Hz; 1H), 1.93-1.87 (m, 1H), 1.37 (s, 9H), 0.86-0.77 (m, 1H), 0.49-0.40 (m, 2H), 0.33-0.30 (m, |
| | | | 1H), 0.18-0.15 (m, 1H). |

### Example 4.2: Synthesis of (S)-4-benzyl-3-(3-(pyridin-3-yl)propanoyl)oxazolidin-2-one (A2-1)

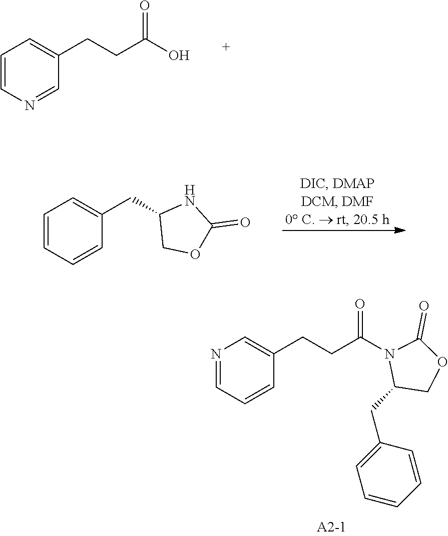 [SMILES: O=C(O)CCc1cccnc1 ; O=C1N[C@@H](Cc2ccccc2)CO1 ; O=C(CCc1cccnc1)N1C(=O)OC[C@@H]1Cc1ccccc1]

To 3-(pyridin-3-yl)propanoic acid (7.56 g, 50 mmol), (S)-4-benzyloxazolidin-2-one (8.86 g, 50.0 mmol) and DMAP (1.833 g, 15.0 mmol) was added DCM (150 mL). The resulting mixture was stirred at rt for 20 min and then cooled to 0° C. The acid was not dissolved to a significant extent. A solution of DIC (10.91 mL, 70.0 mmol) in DCM (10 mL) was added dropwise at 0° C. The acid was still only partially dissolved. DMF (75 mL) was then added and the resulting mixture was stirred for 20.5 h (allowing it to warm up to rt). The solution was concentrated to dryness in vacuo and the residue was suspended in DCM. The suspension was filtered and the residue was washed with DCM. The filtrate was concentrated to dryness in vacuo and the crude product was purified by flash chromatography over silica gel (eluent A: heptane/DIEA (98:2), eluent B: EtOAc/DIEA (98:2)). Pure fractions were combined and concentrated to dryness in vacuo. The resulting residue was dissolved in toluene and the solution was concentrated to dryness in vacuo twice. A2-1 (11.563 g, 37.3 mmol, 74.5% yield) was obtained as a mix of an oil and crystals. Analytical method 10; t_{R}=0.77 min; [M+H]⁺=311.1.

The intermediates shown in Table 18 were prepared according to the procedure used for A2-1 (Example 4.2) from commercially available carboxylic acids or A4-2 (Example 4.3).

TABLE 18

| BB | Structure/Chemical | |
|---|---|---|
| No. | name | LCMS |
| A3-1 | 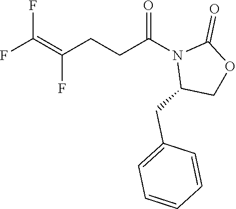 [SMILES: O=C(CCC(F)=C(F)F)N1C(=O)OC[C@@H]1Cc1ccccc1] (S)-4-benzyl-3-(4,5,5- trifluoropent-4- enoyl)oxazolidin-2-one | Analytical method 7 t_{R} = 1.08 min [M + H]⁺ = 314.2 |
| A4-3 | 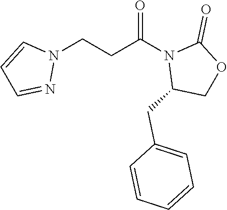 [SMILES: O=C(CCn1cccn1)N1C(=O)OC[C@@H]1Cc1ccccc1] (S)-3-(3-(1H-pyrazol-1- yl)propanoyl)-4- benzyloxazolidin-2-one | Analytical method 5 t_{R} = 0.85 min [M + H]⁺ = 300.2 |
| A5-1 | 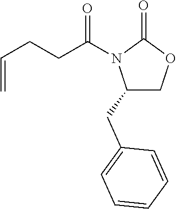 [SMILES: C=CCCC(=O)N1C(=O)OC[C@@H]1Cc1ccccc1] (S)-4-benzyl-3-(pent-4- enoyl)oxazolidin-2-one | Analytical method 5 t_{R} = 1.02 min [M + H]⁺ = 260.0 |
| A7-1 | 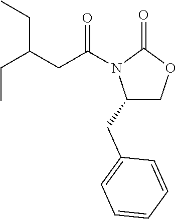 [SMILES: CCC(CC)CC(=O)N1C(=O)OC[C@@H]1Cc1ccccc1] (S)-4-benzyl-3-(3- ethylpentanoyl)oxazolidin- 2-one | Analytical method 5 t_{R} = 1.21 min [M + H]⁺ = 290.3 |

### Example 4.3: Synthesis of 3-(1H-pyrazol-1-yl)propanoic acid (A4-2)

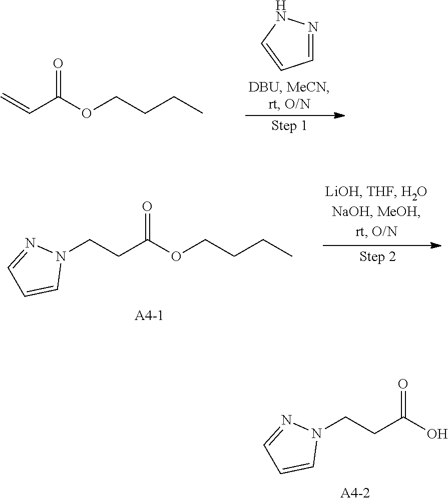

### Step 1. Butyl 3-(1H-pyrazol-1-yl)propanoate (A4-1)

DBU (0.277 mL, 1.836 mmol) was added to a solution of pyrazole (250 mg, 3.67 mmol) and butyl acrylate (789 mL, 5.51 mmol) in MeCN (3.6 mL) in a vial charged with a magnetic stir bar. The reaction was allowed to proceed at rt overnight, after which a peak corresponding to the desired product [M+1]⁺ was observed by LCMS. The reaction was concentrated in vacuo to provide a pale yellow oil. The crude oil was purified by flash chromatography on silica (40 g column, liquid loading (in DCM), eluting with 0-15-100% EtOAc:heptane) to provide A4-1 (723 mg, 100%) as a colorless oil. Analytical method 7; t_{R}=0.85 min; [M+H]⁺=197.3.

### Step 2. 3-(11H-Pyrazol-1-yl)propanoic acid (A4-2)

LiOH (106 mg, 4.42 mmol) was added to a solution of A4-1 (723 mg, 3.68 mmol) in a mixture of THF (7.675 mL) and H₂O (1.535 mL) in a round bottom flask charged with a magnetic stir bar. The reaction was allowed to stir at rt overnight. The reaction mixture was diluted with 10 mL of methanol and treated with 10 mL of 2 N NaOH solution and stirred at rt for 1.5 h, after which LCMS showed complete consumption of starting material. The crude product was acidified with 1 N HCl and concentrated via rotary evaporation to afford A4-2 (mixed with NaCl salt) as a white powder. Analytical method 5; t_{R}=0.15 min; [M−H]⁻=139.2.

### Example 4.4: Synthesis of (R)-2-(4-(tert-butoxy)-2-carboxy-4-oxobutyl)-5-fluoropyridine 1-oxide (A8)

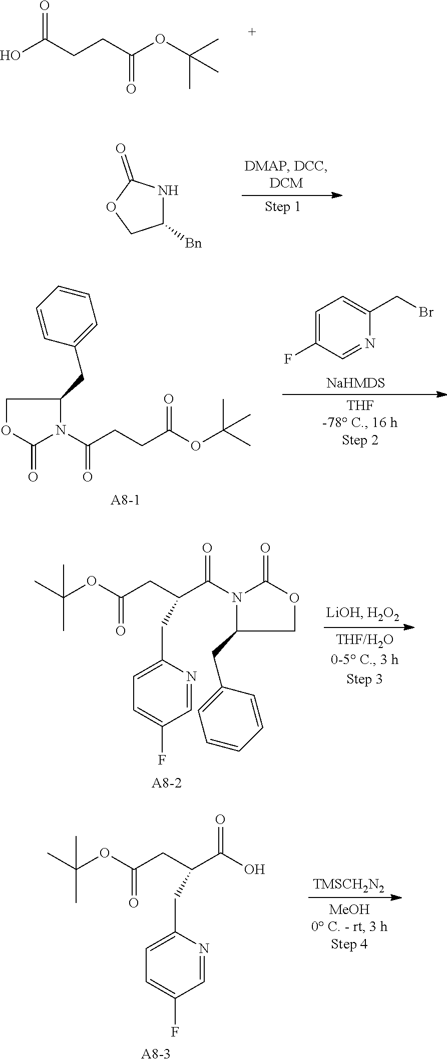 [SMILES: CC(C)(C)OC(=O)CCC(=O)O ; O=C1N[C@H](Cc2ccccc2)CO1 ; CC(C)(C)OC(=O)CCC(=O)N1C(=O)OC[C@H]1Cc1ccccc1 ; Fc1ccc(CBr)nc1 ; CC(C)(C)OC(=O)C[C@@H](Cc1ccc(F)cn1)C(=O)N1C(=O)OC[C@H]1Cc1ccccc1 ; CC(C)(C)OC(=O)C[C@@H](Cc1ccc(F)cn1)C(=O)O]

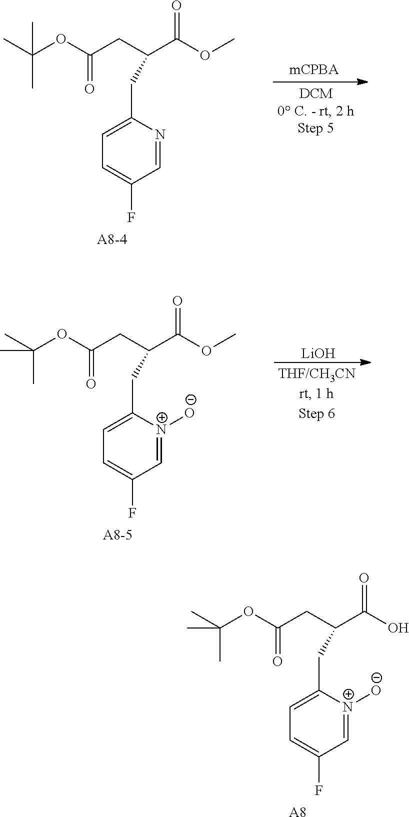

### Step 1. (R)-tert-Butyl 4-(4-benzyl-2-oxooxazolidin-3-yl)-4-oxobutanoate (A8-1)

To a 200 mL round bottom flask containing 4-(tert-butoxy)-4-oxobutanoic acid (5 g, 28.7 mmol), (R)-4-benzyloxazolidin-2-one (6.61 g, 37.3 mmol) and DMAP (0.351 g, 2.87 mmol) in DCM (100 mL) cooled with an ice bath was added DCC (7.7 g, 37.3 mmol) in one portion. The resulting cooled mixture was stirred for 15 min, then warmed to rt and stirred overnight to afford a yellow slurry mixture. The reaction mixture was filtered through a pad of Celite® with DCM and the filtrate was concentrated. The crude product was purified by silica column (eluting with 0→10% EtOAc in DCM) to afford the desired product A8-1 (6.82 g, 71%) as a colorless solid. ¹H NMR (400 MHz, Chloroform-d) δ 7.36 (dd, J=8.0, 6.4 Hz, 2H), 7.33-7.30 (m, 1H), 7.26-7.19 (m, 2H), 4.79-4.64 (m, 1H), 4.32-4.11 (m, 2H), 3.31 (dd, J=13.4, 3.3 Hz, 1H), 3.23 (t, J=6.4 Hz, 2H), 2.80 (dd, J=13.4, 9.6 Hz, 1H), 2.65 (q, J=6.2 Hz, 2H), 1.49 (s, 9H).

### Step 2. (R)-tert-Butyl 4-((R)-4-benzyl-2-oxooxazolidin-3-yl)-3-((5-fluoropyridin-2-yl)methyl)-4-oxobutanoate (A8-2)

To a solution of A8-1 (2 g, 6.00 mmol) in THF (50 mL) was added NaHMDS (1M in THF) (7.20 mL, 7.20 mmol) dropwise at −78° C. The resulting mixture was stirred for 40 min at −78° C. and then 2-(bromomethyl)-5-fluoropyridine (1.3 g, 6.84 mmol) was added dropwise. The reaction mixture was stirred at −78° C. for 16 h, then quenched with NH₄Cl (sat.) and diluted with EtOAc and H₂O. The aqueous layer was extracted twice with EtOAc. The combined organic layers were dried over Na₂SO₄, filtered, and concentrated. The crude product was purified by flash chromatography on silica (eluting with 0-40% EtOAc in heptane) to afford A8-2 as a thick oil (1.6 g, 60%). Analytical method 7; t_{R}=0.94 min; [M+H]⁺=443.3.

### Step 3. (R)-4-(tert-Butoxy)-2-((5-fluoropyridin-2-yl)methyl)-4-oxobutanoic acid (A-8-3)

A8-3 (1.6 g, 3.62 mmol) was prepared according to the procedure used for Example 4.1, Step 3 starting from A8-2 (1.6 g, 3.62 mmol). Analytical method 5; t_{R}=0.43 min; [M+H]⁺=284.3.

### Step 4. (R)-4-tert-Butyl 1-methyl 2-((5-fluoropyridin-2-yl)methyl)succinate (A8-4)

To a solution of (R)-4-(tert-butoxy)-2-((5-fluoropyridin-2-yl)methyl)-4-oxobutanoic acid A8-3 (402 mg, 1.419 mmol) in 12 mL of dry MeOH was added TMSCH₂N₂(2 M in hexane, 3.4 mL, 7.0 mmol) dropwise at 0° C. The resulting mixture was allowed to stir at rt for 2 h, and then quenched with 1 mL of acetic acid. The reaction mixture was diluted with sat. NaHCO₃ and extracted with EtOAc three times. The combined organic layers were dried over Na₂SO₄, filtered, and concentrated under reduced pressure to provide A8-4 (422 mg, 100%), which was used in the next step without purification.

### Step 5. (R)-2-(4-(tert-Butoxy)-2-(methoxycarbonyl)-4-oxobutyl)-5-fluoropyridine 1-oxide (A8-5)

To a solution of A8-4 (422 mg, 1.419 mmol) in DCM (10 mL) at 0° C. was added mCPBA (636 mg, 2.84 mmol). The reaction was stirred at 0° C. for 1 h and then warmed to rt for another 1 h. The reaction was quenched with sat. NaHCO₃ and extracted with EtOAc three times. The combined organic layers were dried over Na₂SO₄, filtered, and concentrated. The resulting residue was purified by flash chromatography on silica gel (0-100% EtOAc/heptane) to afford A8-5 (381 mg, 86%) as light yellow oil. Analytical method 7; t_{R}=0.78 min; [M+H]⁺=315.0.

### Step 6. (R)-2-(4-(tert-Butoxy)-2-carboxy-4-oxobutyl)-5-fluoropyridine 1-oxide, (R)-2-(2,3-dicarboxypropyl)-5-fluoropyridine 1-oxide (A8)

To a solution of A8-5 (381 mg, 1.216 mmol) in THF (9 mL) and ACN (3.00 mL) was added 1 N LiOH aqueous solution (4 mL, 4.00 mmol). The reaction was stirred at rt for 1 h and then concentrated under reduced pressure. The resulting residue was acidified with HCl (4 M aqueous solution) and extracted with EtOAc. The combined organic layers were dried over Na₂SO₄, filtered, and concentrated under reduced pressure. The crude product A8 (364 mg, 100%) was used without purification. Analytical method 7; t_{R}=0.66 min; [M+H]⁺=300.1.

### Example 4.5: Synthesis of 2-((1-(difluoromethyl)-1H-pyrazol-3-yl)methyl)-4-methoxy-4-oxobutanoic acid (A26)

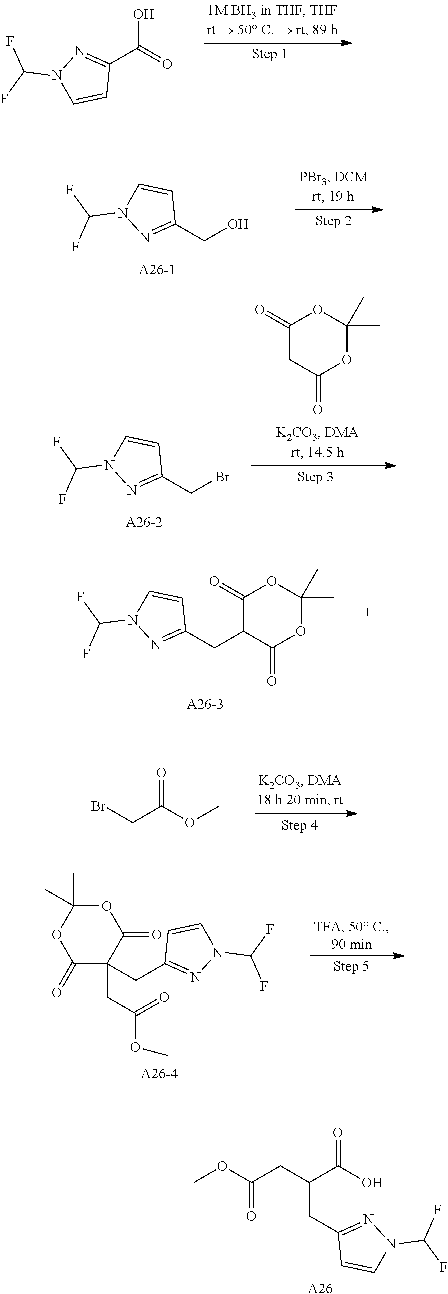

### Step 1. (1-(Difluoromethyl)-1H-pyrazol-3-yl)methanol (A26-1)

To a solution of 1-(difluoromethyl)-1H-pyrazole-3-carboxylic acid (1.84 g, 11.35 mmol) in THF (34 mL) was added 1 M BH₃.THF (34.1 mL, 34.1 mmol). The reaction was stirred for 17 h at rt, then for 5 h at 50° C. Additional 1 M BH₃.THF (5.68 mL, 5.68 mmol) was added and stirring was continued for 4 h at 50° C., and then for 63 h at rt. The reaction was quenched by the addition of H₂O (5 mL). The THF was removed in vacuo and the resulting residue was partitioned between EtOAc (50 mL) and a mixture of 5% aq. NaHCO₃ (25 mL) and 1 M NaOH (10 mL). The organic layer was washed with 1 M NaOH (5 mL) and the combined aqueous layers were washed with EtOAc (2×20 mL). The combined organic layers were washed with brine (10 mL), dried over Na₂SO₄, filtered, and concentrated to dryness in vacuo to afford A26-1 (˜11.35 mmol) as a colorless oil. The crude product was used in the next step without purification. Analytical method 11; t_{R}=0.63 min; [M+H]⁺=149.0.

### Step 2. 3-(Bromomethyl)-1-(difluoromethyl)-1H-pyrazole (A26-2)

To a solution of A26-1 (11.35 mmol) in DCM (20 mL) was added PBr₃ (2.141 mL, 22.70 mmol). The reaction was stirred for 19 h at rt, diluted with DCM (30 mL), and cooled to 0° C. 4 M NaOH (28.4 mL, 114 mmol) was added and the layers were separated. The aqueous layer was washed with DCM (1×20 mL). The combined organic layers were washed with brine (15 mL), dried over Na₂SO₄, filtered, and concentrated to dryness in vacuo. The resulting residue was dissolved in DCM and filtered through silica gel. The filtrate solution was concentrated to dryness in vacuo (40° C., ˜300 mbar) and the crude product was purified by silica gel flash chromatography (eluent A: cyclohexane; eluent B: DCM). Pure fractions were combined and concentrated to dryness in vacuo (40° C., ˜100 mbar) to afford A26-2 (1.45 g, 6.87 mmol, 60.5% yield for 2 steps) as a colorless oil. Analytical method 10; t_{R}=0.79 min; ₁H NMR (400 MHz, Chloroform-d): δ 7.80 (d, J=2.7 Hz, 1H), 7.17 (t, J=60.6 Hz, 1H), 6.54 (d, J=2.6 Hz, 1H), 4.49 (s, 2H).

### Step 3. 5-((1-(Difluoromethyl)-1H-pyrazol-3-yl)methyl)-2,2-dimethyl-1,3-dioxane-4,6-dione (A26-3)

To Meldrum's acid (2.322 g, 16.11 mmol) and K₂CO₃ (2.227 g, 16.11 mmol) was added DMA (15 mL) and the resulting mixture was stirred for 5 min at rt. A solution of A26-2 (0.680 g, 3.22 mmol) in DMA (2 mL) was added and the reaction was stirred for 14.5 h at rt, and then partitioned between EtOAc (100 mL) and 1 M aq. HCl (50 mL). The organic layer was washed with 5% aq. KHSO₄ (15 mL) and the combined aqueous layers were extracted with EtOAc (30 mL). The combined organic layers were washed with brine (20 mL), dried over Na₂SO₄, filtered, and concentrated to dryness in vacuo. The crude product was purified by flash chromatography over silica gel (eluting with 0-100% EtOAc in Heptane). A26-3 (assumed to be 3.22 mmol) was obtained as a colorless oil and used in the next step without purification. Analytical method 10; t_{R}=0.71 min; [M+H]⁺=275.1.

### Step 4. Methyl 2-(5-((1-(difluoromethyl)-1H-pyrazol-3-yl)methyl)-2,2-dimethyl-4,6-dioxo-1,3-dioxan-5-yl)acetate (A26-4)

To A26-3 (3.22 mmol) dissolved in DMA (15 mL) was added K₂CO₃ (1.334 g, 9.65 mmol) and methyl 2-bromoacetate (0.889 mL, 9.65 mmol) and the resulting suspension was stirred for 3 h 20 min at rt. Additional K₂CO₃ (1.334 g, 9.65 mmol) and methyl 2-bromoacetate (0.889 mL, 9.65 mmol) were added and the resulting mixture was stirred at rt for 15 h. The reaction was partitioned between EtOAc (60 mL) and H₂O (15 mL). The organic layer was washed with 5% aq. NaHCO₃ (3×10 mL) and brine (10 mL), dried over Na₂SO₄, filtered, and concentrated to dryness in vacuo to afford A26-4 (assumed to be 3.22 mmol) as a yellowish oil/wax. The crude product was used in the next step without purification. Analytical method 10; t_{R}=0.87 min; [M+H]⁺=347.2.

### Step 5. 2-((1-(Difluoromethyl)-1H-pyrazol-3-yl)methyl)-4-methoxy-4-oxobutanoic acid (A26)

A26-4 (3.22 mmol) was dissolved in TFA (12 mL). The solution was stirred for 90 min at 50° C., H₂O (2 mL) was added and the reaction was concentrated to dryness in vacuo. The crude product was purified by flash chromatography over Polygoprep 60-50 C18 (eluent A: 0.1% TFA in H₂O and eluent B: ACN). Pure fractions were combined and ACN was removed in vacuo. The product was extracted with EtOAc (5×50 mL) and the organic layer was concentrated to dryness to afford A26 (511 mg, 1.949 mmol, 60.5% yield for 3 steps) as a slightly yellow oil. Analytical method 10; t_{R}=0.61 min; [M+H]⁺=263.1.

### Example 4.6: Synthesis of 2-(bicyclo[1.1.1]pentan-1-ylmethyl)-4-(tert-butoxy)-4-oxobutanoic acid (A27)

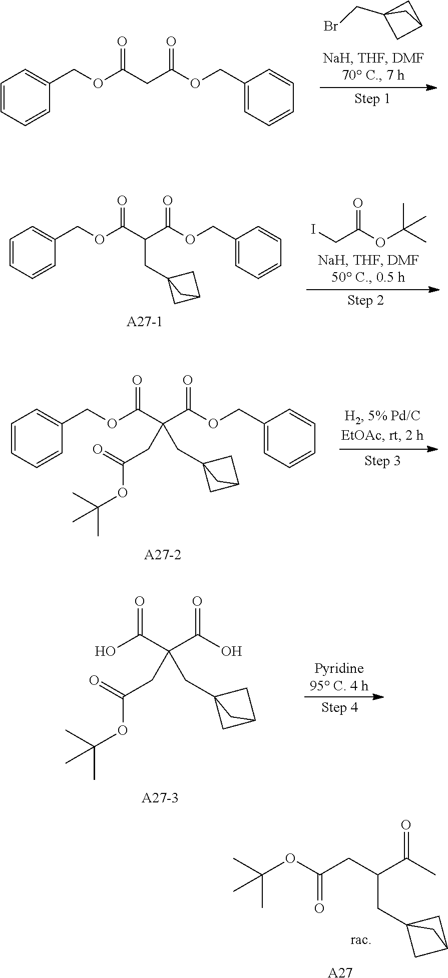

### Step 1. Dibenzyl 2-(bicyclo[1.1.1]pentan-1-ylmethyl)malonate (A27-1)

To benzyl malonate (3574 mg, 12.57 mmol) stirred in THF (23 mL) at 0° C. was added sodium hydride (503 mg, 12.57 mmol, 60% in mineral oil) in two portions. The reaction mixture was stirred at rt for 20 min and then 1-(bromo methyl)bicyclo[1.1.1.]pentane (964 mg, 5.99 mmol) in THF (3 mL) and DMF (19 mL) were added at 0° C. The reaction mixture was stirred at at 0° C. for 30 min and then warmed to 70° C. and stirred for 7 h. The progress of the reaction was monitored by GC. The reaction mixture was diluted with TBME (100 mL) and acidified with 20 mL 10% citric acid aqueous solution. The aqueous solution was washed with TBME (100 mL). The combined organic phases were washed with 20 mL 10% citric acid, 5% aq. NaHCO₃ (50 mL×2) and brine (50 mL), dried over MgSO₄, filtered, and concentrated. The crude product was purified via normal phase chromatography using silica gel (100-200 mesh) eluting with 5→20% ethyl acetate in heptane to afford compound A27-1 (1220 mg, 53%) as a clear oil. Analytical method 10; t_{R}=1.46 min; [M+H]⁺=365.3.

### Step 2. 2,2-Dibenzyl 1-tert-butyl 3-(bicyclo[1.1.1]pentan-1-yl)propane-1,2,2-tricarboxylate (A27-2)

A27-1 (1220 mg, 3.35 mmol) was dissolved in THF (16 mL) at rt and the resulting solution was cooled to about 4° C. (ice bath cooling). NaH (170 mg, 4.25 mmol; 60% in mineral oil) was then added cautiously. The cooling bath was removed and the mixture was stirred for 20 minutes at rt. The mixture was then diluted with DMF (w8 mL) and tert-butyl 2-iodoacetate (1200 mg, 4.71 mmol) in THF (4 mL) was added. The resulting mixture was stirred at 60° C., and after 10 minutes a thick suspension formed. The mixture was diluted with 2 mL THF and then stirred for 2 hours at 60° C. The progress of the reaction was monitored by LCMS. The mixture was cooled to rt, diluted with TBME, and washed twice with 5% aq. citric acid and once with 5% aq. NaHCO₃. The combined aqueous phases were extracted with TBME. The combined organic phases were dried over MgSO₄, filtered, and concentrated. The crude compound was purified via normal phase chromatography using silica gel (100-200 mesh) column chromatography eluting with 5-20% ethyl acetate in heptane as a solvent to afford compound A27-2 (1400 mg, 83%) as a clear oil. Analytical method 10; t_{R}=1.58 min; [M+H]⁺=479.4. ¹H NMR (400 MHz, CDCl₃): δ 7.20-7.31 (m, 10H), 5.06-5.10 (s, 4H), 3.02 (s, 2H), 2.35 (s, 1H), 2.25 (s, 2H), 1.65 (s, 6H), 1.47 (s, 9H).

### Step 3. 2-(Bicyclo[1.1.1]pentan-1-ylmethyl)-2-(2-(tert-butoxy)-2-oxoethyl)malonic acid (A27-3)

To A27-2 (1000 mg, 2.09 mmol) dissolved in EtOAc (60 mL) was added Pd/C (100 mg, 5% wet based) and the resulting mixture was stirred at rt for 2 hours under a hydrogen atmosphere (balloon). The reaction mixture was then filtered through a Celite® pad and the filtrate was concentrated to dryness to afford compound A27-3 (598 mg, 96%) as white solid which was used as is for the next reaction without purification. MS (flow injection) [M−H]⁻=297.2. ¹H NMR (400 MHz, DMSO-d₆): δ 13.20-12.80 (bs, 2H), 2.82 (s, 2H), 2.40 (s, 1H), 2.06 (s, 2H), 1.68 (s, 6H), 1.38 (s, 9H).

### Step 4. 2-(Bicyclo[1.1.1]pentan-1-ylmethyl)-4-(tert-butoxy)-4-oxobutanoic acid (A27)

A27-3 (590 mg, 2.09 mmol) was dissolved in pyridine (20 mL). The resulting solution was stirred at 95° C. for 4 hours and then evaporated to dryness (50° C., 0.1 mbar). The crude product was diluted in TBME and washed with 5% citric acid aqueous solution. The organic phase was dried over MgSO₄, filtered, and concentrated to dryness to afford compound A27 (508 mg, 99%). MS (flow injection) [M−H]⁻=253.3. ¹H NMR (400 MHz, Chloroform-d) δ 2.88-2.76 (m, 1H), 2.60 (dd, J=16.4, 9.0 Hz, 1H), 2.44 (s, 1H), 2.44-2.36 (m, 1H), 1.87 (dd, J=14.4, 7.1 Hz, 1H), 1.72 (s, 6H), 1.64 (dd, J=14.4, 6.2 Hz, 1H), 1.44 (s, 9H).

### Example 4.7: 4-Methoxy-4-oxo-2-((2-oxopyridin-1(2H)-yl)methyl)butanoic acid (A28)

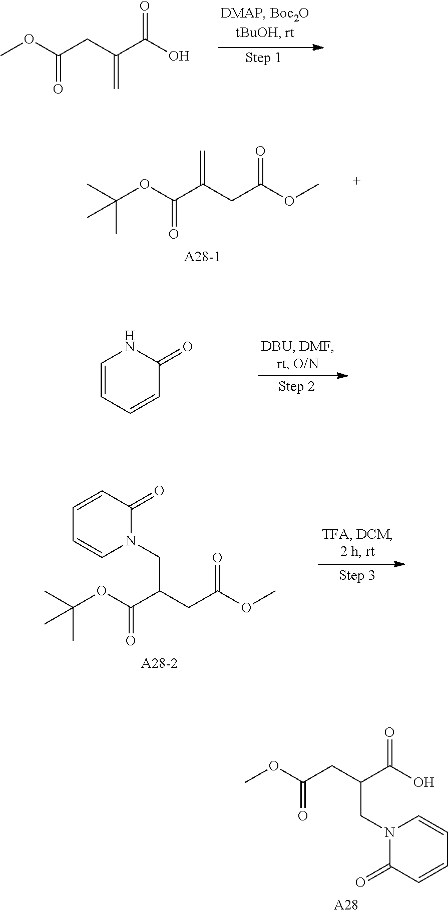

### Step 1. 1-tert-Butyl 4-methyl 2-methylenesuccinate (A28-1)

The title compound was prepared according to procedure in PCT Int. Appl., 2006/074940, 20 Jul. 2006 starting from 4-methoxy-2-methylene-4-oxobutanoic acid (1 g, 6.4 mmol) to afford A28-1 (604 mg, 43%). The crude material taken on to next step without purification.

### Step 2. 1-tert-Butyl 4-methyl 2-((2-oxopyridin-1(2H)-yl)methyl)succinate (A28-2)

To a flask containing A28-1 (320 mg, 1.60 mmol) and pyridin-2(1H)-one (152 mg, 1.60 mmol) was added DMF (1.1 mL) and the resulting mixture was stirred at rt until all of the solid dissolved to give a clear solution. DBU (0.241 mL, 1.60 mmol) was then added dropwise and the resulting mixture stirred for overnight. The reaction mixture was taken up in EtOAc and washed with brine (3×). The organic phase was dried over sodium sulfate, filtered, and concentrated to afford a crude oil. The crude product was by normal phase chromatography using a silica gel column (eluting with 0-10% DCM/MeOH) to afford A28-2 (424 mg, 82%). Analytical method 7; t_{R}=0.75 min; [M+H]⁺=295.8.

### Step 3. 4-Methoxy-4-oxo-2-((2-oxopyridin-1(2H)-yl)methyl)butanoic acid (A28)

To A28-2 (424 mg, 1.44 mmol) in DCM (2 mL) under N₂ was added TFA (2 mL) and the resulting mixture was stirred for 2 h and then concentrated under vacuum to afford A28 (343 mg, crude). Analytical method 7; t_{R}=0.45 min; [M+H]⁺=240.2.

### Example 5: Building Block B (BB B)-D

### Example 5.1: Synthesis of tert-butyl ((1S,2S)-2-(methylamino)cyclohexyl)carbamate (B1)

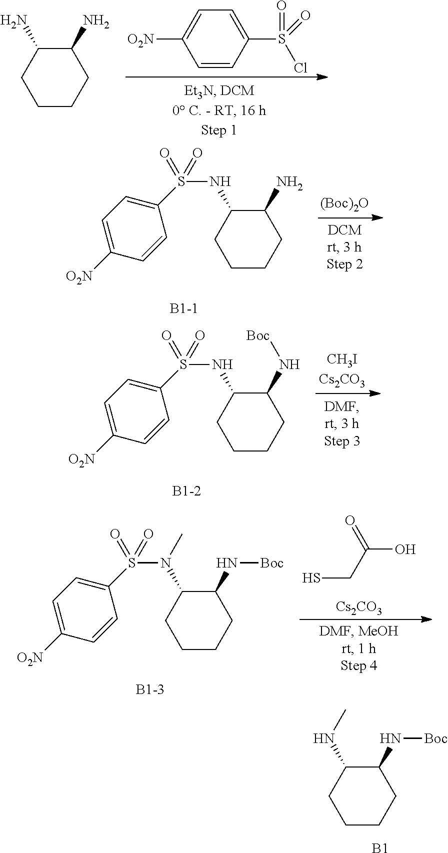

### Step 1. N-((1S,2S)-2-Aminocyclohexyl)-4-nitrobenzenesulfonamide (B1-1)

4-Nitrobenzenesulfonyl chloride (23.29 g, 105 mmol) was added to a solution of (1S,2S (+)-1,2-diaminocyclohexane (12 g, 105 mmol) and triethylamine (21.97 mL, 158 mmol) in DCM (200 mL) at 0° C. and stirred at the same temperature for 30 min. The reaction was slowly warmed to room temperature and stirred for 16 h. The progress of the reaction was monitored by TLC (80% ethyl acetate in petroleum ether). The reaction mixture was concentrated and diluted with water and solid was precipitated. The precipitate was filtered and washed with an excess amount of water and EtOAc and dried under vacuum to afford compound B1-1 (17.66 g, 56%) as a white solid. Analytical method 7; t_{R}=0.74 min; [M+H]⁺=300.2. ¹HNMR (300 MHz, CDCl₃): δ 8.33-8.31 (d, J=8.8 Hz, 2H), 8.05-8.02 (d, J=8.8 Hz, 2H), 6.19-6.18 (d, J=4.8 Hz, 1H), 4.40-4.38 (d, J=7.6 Hz, 1H), 3.35-3.33 (d, J=10.4 Hz, 1H), 2.97-2.91 (m, 1H), 2.01-1.93 (m, 2H), 1.73-1.68 (m, 2H), 1.67-1.65 (d, J=6.8 Hz, 1H), 1.52-1.47 (m, 9H), 1.29-1.16 (m, 4H).

### Step 2. tert-Butyl ((1S,2S)-2-((4-nitrophenyl)sulfonamido)cyclohexyl)carbamate (B1-2)

Boc-anhydride (13.70 mL, 59.0 mmol) was added to a stirred solution of compound B1-1 (17.66 g, 59.0 mmol) in DCM (200 mL) and stirred for 3 h. The progress of the reaction was monitored by TLC (50% ethyl acetate in petroleum ether). The reaction mixture was concentrated under reduced pressure to get compound B1-2 (23.5 g, 100%) as a white solid. Analytical method 7; t_{R}=1.14 min; [M-Boc+H]⁺=299.9. ¹HNMR (400 MHz, CDCl₃): δ 8.33-8.31 (d, J=9.2 Hz, 2H), 8.04-8.02 (d, J=8.8 Hz, 2H), 6.17-6.16 (d, J=4.8 Hz, 1H), 4.38-4.36 (d, J=7.2 Hz, 1H), 3.35-3.32 (m, 1H), 2.96-2.91 (m, 1H), 2.02-1.92 (m, 2H), 1.73-1.65 (m, 2H), 1.52-1.46 (m, 6H), 1.36-1.27 (m, 9H), 1.28-1.21 (m, 4H).

### Step 3. tert-Butyl ((1S,2S)-2-((N-methyl-4-nitrophenyl)sulfonamido)cyclohexyl)carbamate (B1-3)

Methyl iodide (18.19 mL, 294 mmol) was added to a stirred solution of compound B1-2 (23.5 g, 58.8 mmol) and Cs₂CO₃ (47.9 g, 147 mmol) in DMF (200 mL) and stirred at the same temperature for 3 h. The progress of the reaction was monitored by TLC (40% ethyl acetate in petroleum ether). The reaction mixture was diluted with water (300 mL) and extracted with ethyl acetate (300 mL). The organic layer was washed with water and brine and dried over anhydrous Na₂SO₄. The organic layer was concentrated under reduced pressure to get crude compound. The crude compound was purified via normal phase chromatography using silica gel (100-200 mesh) column chromatography eluting with 0→30% ethyl acetate in petroleum ether as a solvent to afford compound B1-3 (21.1 g, 89%) as a white solid. Analytical method 7; t_{R}=1.22 min; [M-Boc]⁺=313.9. ¹HNMR (300 MHz, CDCl₃): δ 8.36-8.35 (d, J=6.8 Hz, 2H), 8.00-7.97 (d, J=8.8 Hz, 2H), 4.48 (s, 1H), 4.14-4.09 (m, 1H), 3.56-3.54 (d, J=6.4 Hz, 2H), 2.88 (s, 3H), 2.12-2.10 (m, 1H), 2.04 (s, 1H), 1.72-1.70 (d, J=7.6 Hz, 2H), 1.41 (s, 9H), 1.27-1.23 (m, 4H).

### Step 4. tert-Butyl ((1S,2S)-2-(methylamino)cyclohexyl)carbamate (B1)

A mixture of compound B1-3 (1.55 g, 3.75 mmol), Cs₂CO₃ (8.55 g, 26.2 mmol) and 2-mercaptoacetic acid (1.524 mL, 14.99 mmol) in a mixture of DMF:MeOH (1:1, 12 mL) was stirred at rt for 1 h. The progress of the reaction was monitored by TLC (10% MeOH in DCM). The reaction mixture was diluted with water (100 mL) and ethyl acetate (100 mL). The aqueous layer was extracted with ethyl acetate (100 mL). The organic layers were combined, washed with brine and dried over Na₂SO₄. The organic layer was concentrated under reduced pressure to get crude product. The crude product was purified via normal phase chromatography by silica gel (100-200 mesh) column chromatography eluting with 0→5% MeOH in DCM as an eluent to afford a pale yellow solid B1 (860 mg, 100%). Analytical method 7; t_{R}=0.75 min; [M+H]⁺=229.3. ¹H NMR (300 MHz, DMSO-d₆): δ 6.26-6.18 (m, 1H), 3.08-3.06 (m, 1H), 2.35 (s, 3H), 2.15-2.12 (m, 1H), 1.93-1.86 (m, 1H), 1.77 (m, 1H), 1.61-1.58 (m, 2H), 1.37 (s, 9H), 1.23-0.96 (m, 5H).

### Example 5.2: Synthesis of (S)—N-(2-(methylamino)propyl)-4-nitrobenzenesulfonamide (B4)

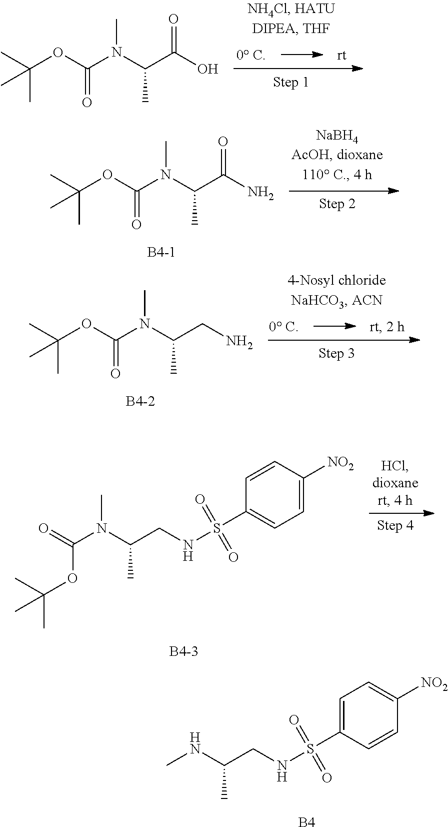

### Step 1. tert-Butyl (S)-(1-amino-1-oxopropan-2-yl)(methyl)carbamate (B4-1)

To a stirred solution of N-Me-Boc-Ala-OH (1000.0 g, 4.92 mol) in THF (6 L) was added DIEA (3179.0 g, 24.6 mol) at 0° C. After 5 min HATU (2058.0 g, 294 mmol) was added in one portion and stirring was continued at the same temperature for 15 min. Solid ammonium chloride (1316.0 g, 24.6 mol) was then added and the mixture was stirred overnight at room temperature. A large amount of precipitate was formed which was filtered through a disposable frit, washing multiple times with THF until most of the solid dissolved. The filtrate was evaporated under reduced pressure to get crude product. The crude mass was diluted with water (2 L) and extracted by petroleum ether (2×2.5 L) to remove the non-polar impurities. The water part was then extracted with 40% EtOAc in petroleum ether several times. Then all organic parts were combined, dried over anhydrous Na₂SO₄, filtered & evaporated under reduced pressure to get compound B4-1 (650.0 g, 65%). ¹H NMR (400 MHz, DMSO-d₆) □ ppm 6.70-7.47 (m, 2H), 4.48-4.49 (m, 1H), 4.11-4.82 (m, 1H), 3.08 (s, 1H), 2.72 (s, 3H), 1.39 (br. s., 9H), 1.18-1.29 (m, 3H), 1.11 (s, 1H).

### Step 2. tert-Butyl (S)-(1-aminopropan-2-yl)(methyl)carbamate (B4-2)

To a stirred solution of B4-1 (100 g, 0.49 mol.) in 1,4-dioxane (500 mL) was added NaBH₄ (56.4 g, 1.48 mol) under argon. Acetic acid (90 mL) in dioxane (200 mL) was added drop wise to the mixture so as to maintain a gentle effervescence, with a bubbler attached as a vent. Upon complete addition of the acid, a reflux condenser was attached, and the mixture was heated to 110° C. for 4 h. The mixture was removed from heat and allowed to come to room temperature. The reaction mass was quenched by ice & acidified with 2 N HCl. The solution was extracted with EtOAc and the aqueous part was basified up to pH 13-14 with 50% NaOH. The solution was then extracted with diethyl ether, dried over anhydrous Na₂SO₄, filtered & evaporated under reduced pressure to get crude yellow oil B4-2 (55.3 g, 59%). The crude compound was used for the next step without purification.

### Step 3. tert-Butyl (S)-methyl(1-((4-nitrophenyl)sulfonamido)propan-2-yl)carbamate (B4-3)

To a stirred solution of crude B4-2 (300.0 g, 1.59 mol) in ACN (1500 mL) was added NaHCO₃ (406.9 g, 4.78 mol), followed by nosyl chloride (388.9 g, 1.75 mol) at 0° C. The mixture was stirred at room temperature for 2 h. The reaction solution was slowly quenched with ice water (2000 mL) and stirred for 2 h until white precipitate formed. Solids were filtered, washed with water and petroleum ether and dried under vacuum to give a light yellow solid crude B4-3 (451.2 g, 75%). The crude compound was used for next step without purification.

### Step 4. (S)—N-(2-(Methylamino)propyl)-4-nitrobenzenesulfonamide (B4)

To a stirred solution of crude compound B4-3 (450.0 g, 1.2 mol) in 1,4-dioxane (2000 mL) was added HCl (4 M in 1,4-dioxane, 2000 mL) and the reaction mixture was stirred at rt for 4 h. Solids precipitated during the reaction, which was filtered, washed with diethyl ether and dried under vacuum to get the amine as an HCl-salt. The crude solid mass was washed several times by n-pentane and finally dried to get pure compound B4 (264.3 g, 80%) as a white solid. Analytical method 7; t_{R}=0.69 min; [M+H]⁺=274.2. ¹H NMR (400 MHz, DMSO-d₆) b ppm 1.18 (d, J=6.57 Hz, 3H) 2.47-2.55 (m, 4H) 2.89-3.13 (m, 2H) 3.14-3.26 (m, 1H) 8.03-8.16 (m, 2H) 8.37-8.52 (m, 2H).

The diamine building blocks shown in Table 19 below were prepared according to the procedure used for B1 (Example 5.1), B4 (Example 5.2), and B4-2 (Example 5.2, Step 1-2).

TABLE 19

| BB | | | Synthesis procedure/ |
|---|---|---|---|
| No. | Structure | Name | LCMS/NMR |
| B2 | 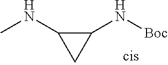 | tert-butyl ((1R*,2S*)- 2-(methylamino)cyclo- propyl)carbamate | B1 (Example 5.1)-intermediate corresponding to B1-2 was obtained by nosylation of tert- butyl (cis)-2- aminocyclopropyl)carbamate/ |
| | | | Analytical method 7 |
| | | | t_{R} = 0.40 min |
| | | | [M + H]⁺ = 187.3 |
| B3 | 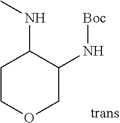 [SMILES: CNC1CCOCC1NC(=O)OC(C)(C)C] | tert-butyl ((3S*,4R*)- 4-(methylamino)tetra- hydro-2H-pyran-3- yl)carbamate | B1 (Example 5.1)-intermediate corresponding to B1-2 was obtained by nosylation of tert- butyl ((1R*,2R*)-2- aminocyclohexyl)carbamate/ Analytical method 7 t_{R} = 0.44 min [M + H]⁺ = 231.3. |
| B5 | 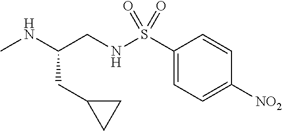 [SMILES: CN[C@H](CNS(=O)(=O)c1ccc([N+](=O)[O-])cc1)CC1CC1] | (S)-N-(3-cyclopropyl- 2-(methylamino) propyl)-4-nitrobenzene sulfonamide | B4 (Example 5.2)- Analytical method 7 t_{R} = 0.61 min [M + H]⁺ = 314.2 |
| B6 | 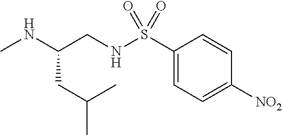 [SMILES: CN[C@H](CNS(=O)(=O)c1ccc([N+](=O)[O-])cc1)CC(C)C] | (S)-N-(4-methyl-2- (methylamino)pentyl)- 4-nitrobenzene- sulfonamide | B4 (Example 5.2)- Analytical method 7 t_{R} = 0.64 min [M + H]⁺ = 316.0 |
| B7 | 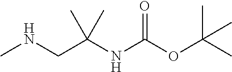 | tert-butyl (2-methyl-1- (methylamino)propan- 2-yl)carbamate | B4-2 (Example 5.2, Step 1-2)- starting from 2-((tert- butoxycarbonyl)amino)-2- methylpropanoic acid/ Analytical method 16 |
| | | | t_{R} = 0.53 min |
| | | | [M + H]⁺ = 202.2/¹H NMR |
| | | | (400 MHz, DMSO-d₆) |
| | | | δ ppm 6.13 (s, 1H), 2.30 (s, 4H), |
| | | | 1.37 (s, 12H), 1.24 (d, J = 4.2 |
| | | | Hz, 3H), 1.16 (s, 8H). |
| B8-1 | 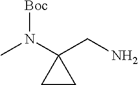 | tert-butyl (1- (aminomethyl)cyclo- propyl)(methyl) carbamate | B4 (Example 5.2)-starting from 1-((tert- butoxycarbonyl)(methyl)amino) cyclopropane-1-carboxylic acid. |
| B9 | 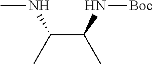 | tert-butyl ((2S,3S)-3- (methylamino)butan-2- yl)carbamate | B1 (Example 5.1)-starting from B9-5 (see Example 5.4)/ ¹H NMR (400 MHz, DMSO-d6) δ ppm 0.89 (3H, d, J = 6.36 Hz) |
| | | | 0.95 (3H, d, J = 6.85 Hz) 1.31- |
| | | | 1.46 (9H, m) 2.24-2.32 (3H, |
| | | | m) 3.42-3.58 (1H, m) 6.60 (1H, |
| | | | br d, J = 8.31 Hz). |
| B10 | 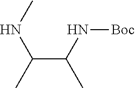 | tert-butyl (3- (methylamino) butan- 2-yl)carbamate | B1 (Example 5.1)-starting from butane-2,3-diamine dihydrochloride. Step 1 and Step 2 were done in reverse order/ Analytical method 8 t_{R} = 0.96 min [M + H]⁺ = 203.2 |
| B16 | 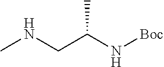 | tert-butyl (S)-(1- (methylamino)propan- 2-yl)carbamate | B4-2 (Example 5.2, Step 1-2)- starting from Boc-Ala-OH and methylamine/ ¹H NMR (400 MHz, DMSO-d₆) δ 3.57-3.45 (m, 1H), 2.43 (dd, J = |
| | | | 11.7, 6.8 Hz, 1H), 2.33 (dd, J = |
| | | | 11.8, 6.4 Hz, 1H), 2.25 (s, 3H), |
| | | | 1.38 (s, 9H), 0.99 (d, J = 6.4 Hz, |
| | | | 3H). |
| B17 | 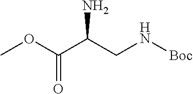 | methyl (S)-2-amino-3- ((tert-butoxycarbonyl) amino)propanoate | Commercially available |
| B18 | 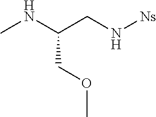 | (R)-N-(3-methoxy-2- (methylamino)propyl)- 4-nitrobenzene- sulfonamide | B4 (Example 5.2)-starting from Boc-Ser(Me)-OH/Analytical method 5 t_{R} = 0.68 min [M + H]⁺ = 304.1 |
| B19 | 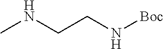 | tert-butyl (2- (methylamino)ethyl) carbamate hydro- chloride | Commercially available |

### Example 5.2: Synthesis of (S)—N-(2-(methylamino)propyl)-4-nitrobenzenesulfonamide (B34)

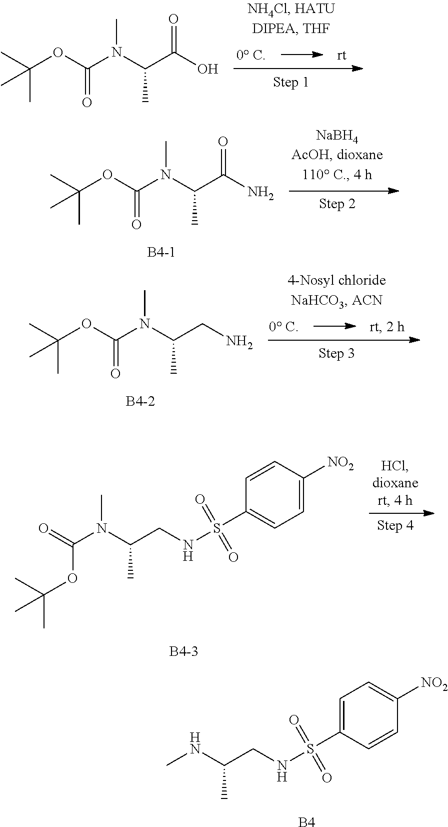

### Step 1. tert-Butyl (S)-(1-amino-1-oxopropan-2-yl)(methyl)carbamate (1B4-1)

To a stirred solution of N-Me-Boc-Ala-OH (1000.0 g, 4.92 mol) in THF (6 L) was added DIEA (3179.0 g, 24.6 mol) at 0° C. After 5 min HATU (2058.0 g, 294 mmol) was added in one portion and stirring was continued at 0° C. for 15 min. Solid ammonium chloride (1316.0 g, 24.6 mol) was then added and the mixture was stirred overnight at room temperature. A large amount of precipitate was formed which was filtered through a disposable frit, washing multiple times with THF until most of the solid dissolved. The filtrate was evaporated under reduced pressure to provide the crude product. Water was added (2 L) to the crude product and the aqueous layer was extracted by petroleum ether (2×2.5 L) to remove the non-polar impurities. The aqueous layer was then extracted with 40% EtOAc in petroleum ether several times. The combined organic phases were dried over anhydrous Na₂SO₄, filtered, and evaporated under reduced pressure to provide compound B4-1 (650.0 g, 65%) which was carried onto the next step without purification. ¹H NMR (400 MHz, DMSO-d₆) δ ppm 6.70-7.47 (m, 2H), 4.48-4.49 (m, 1H), 4.11-4.82 (m, 1H), 3.08 (s, 1H), 2.72 (s, 3H), 1.39 (br. s., 9H), 1.18-1.29 (m, 3H), 1.11 (s, 1H).

### Step 2. tert-Butyl (S)-(1-aminopropan-2-yl)(methyl)carbamate (B4-2)

To a stirred solution of B4-1 (100 g, 0.49 mol.) in 1,4-dioxane (500 mL) under an argon atmosphere was added NaBH₄ (56.4 g, 1.48 mol). Acetic acid (90 mL) in dioxane (200 mL) was added drop wise to the mixture so as to maintain a gentle effervescence, with a bubbler attached to the reaction mixture as a vent. Upon complete addition of the acid, a reflux condenser was attached, and the resulting mixture was heated to 110° C. for 4 h. The reaction mixture was removed from heat, allowed to warm to room temperature and then quenched with ice and acidified with 2 N HCl. The mixture was extracted with EtOAc and the aqueous phase was basified up to pH 13-14 with 50% NaOH. The basified aqueous layer was then extracted with diethyl ether, dried over anhydrous Na₂SO₄, filtered, and evaporated under reduced pressure to afford B4-2 (55.3 g, 59%) as a crude yellow oil, which was used for the next step without purification.

### Step 3. tert-Butyl (S)-methyl(1-((4-nitrophenyl)sulfonamido)propan-2-yl)carbamate (B4-3)

To a stirred solution of crude B4-2 (300.0 g, 1.59 mol) in ACN (1500 mL) was added NaHCO₃ (406.9 g, 4.78 mol), followed by nosyl chloride (388.9 g, 1.75 mol) at 0° C. The mixture was stirred at room temperature for 2 h. The reaction solution was slowly quenched with ice water (2000 mL) and stirred for 2 h until white precipitate formed. Solids were filtered, washed with water and petroleum ether and dried under vacuum to give a light yellow solid crude B4-3 (451.2 g, 75%). The crude compound was used for next step without purification.

### Step 4. (S)—N-(2-(Methylamino)propyl)-4-nitrobenzenesulfonamide (B4)

To a stirred solution of B4-3 (450.0 g, 1.2 mol) in 1,4-dioxane (2000 mL) was added HCl (4 M in 1,4-dioxane, 2000 mL) and the resulting mixture was stirred at rt for 4 h. The solids, which precipitated during the reaction, were filtered, washed with diethyl ether, and dried under vacuum to afford the amine as an HCl-salt. The crude product was washed several times by n-pentane and then dried to provide pure B4 (264.3 g, 80%) as a white solid. Analytical method 7; t_{R}=0.69 min; [M+H]⁺=274.2. ¹H NMR (400 MHz, DMSO-d₆) b ppm 1.18 (d, J=6.57 Hz, 3H) 2.47-2.55 (m, 4H) 2.89-3.13 (m, 2H) 3.14-3.26 (m, 1H) 8.03-8.16 (m, 2H) 8.37-8.52 (m, 2H).

### Example 5.3: Synthesis of (9H-fluoren-9-yl)methyl ((1-(methylamino)cyclopropyl)methyl) carbamate hydrochloride (B8)

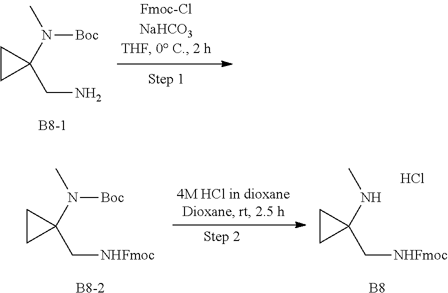

### Step 1. tert-Butyl (1-(((((9H-fluoren-9-yl)methoxy)carbonyl)amino)methyl)cyclopropyl)(methyl)carbamate (B8-2)

To a stirred solution of B8-1 (1.015 g, 5.07 mmol) in THF (24 mL) was added NaHCO₃ (15.2 mL, 15.2 mmol), followed by (9H-fluoren-9-yl)methyl carbonochloridate (1.573 g, 6.08 mmol) at 0° C. and the resulting mixture was stirred at room temperature for 2 h and then concentrated. The crude material was taken up in DCM (40 mL) and the organic phase was washed with water (2×30 mL). The combined organic phases were dried (MgSO₄), filtered, and concentrated under reduced pressure. The crude product was purified via normal phase chromatography on an 80 g silica column eluting with a DCM/EtOAc gradient to yield B8-2 (1.04 g, 49%) as a white foam. Analytical method 7; t_{R}=1.71 min; [M-Boc]⁺=323.1.

### Step 2. (9H-Fluoren-9-yl)methyl ((1-(methylamino)cyclopropyl)methyl)carbamate hydrochloride (B8)

To a stirred solution of B8-2 (196 mg, 0.464 mmol) in 1,4-dioxane (3 mL) was added 4 M HCl in dioxane (1.74 mL, 6.96 mmol). The resulting mixture was stirred at rt for 2.5 h and then concentrated in vacuo to yield B8 (166 mg, 100%) as a white solid. Analytical method 7; t_{R}=1.03 min; [M+H]⁺=323.1.

### Example 5.4: Synthesis of tert-butyl ((2S,3S)-3-((4-nitrophenyl)sulfonamido)butan-2-yl)carbamate (B39-5)

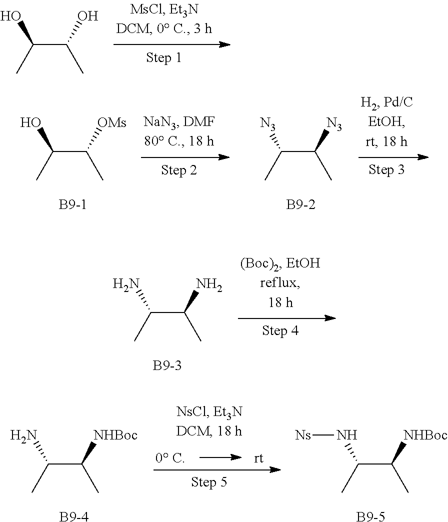

### Step 1. ((2R,3R)-Butane-2,3-diyl dimethanesulfonate (B9-1)

To a solution of (2R,3R)-butane-2,3-diol (25 g, 278 mmol) in DCM (1 L) was added Et₃N (70 g, 694 mmol). MsCl (80 g, 694 mmol) was then added dropwise keeping the temperature below 10° C. The resulting mixture was allowed to stir at 0° C. and then slowly warmed to rt over 3 h. The reaction mixture was poured into 1 N HCl (500 mL) and the layers were separated. The organic layer was washed with sat. aq. NaHCO₃ (500 mL), water, and brine, dried over Na₂SO₄, filtered, and concentrated under reduced pressure. The crude residue was purified by silica gel chromatography eluting with 1:1 PE: EA to afford compound B9-1 (60 g, 87%). ¹H NMR (300 MHz, CDCl₃): δ 4.82-4.73 (m, 2H), 3.07 (s, 6H), 1.46 (d, J=6 Hz, 6H).

### Step 2. (2S,3S)-2,3-Diazidobutane (B9-2)

To a solution of B9-1 (60 g, 244 mmol) in DMF (500 mL) was added NaN₃ (40 g, 615 mmol) and the resulting mixture was stirred at 80° C. overnight. H₂O was added to the mixture and the aqueous phase was extracted with Et₂O (3×1 L). The combined organic phases were dried and concentration dried over Na₂SO₄, filtered, and concentrated under reduced pressure. B9-2 (34 g, crude) was carried onto the next step without purification. ¹H NMR (300 MHz, CDCl₃): δ 3.65-3.37 (m, 2H), 1.48-1.30 (m, 6H).

### Step 3. (2S,3S)-Butane-2,3-diamine (B9-3)

To a solution of B9-2 (17 g, 121.4 mmol) in EtOH (600 mL) was added Pd/C (1.7 g) and the resulting mixture was stirred at rt overnight under an atmosphere of H₂. The reaction mixture was filtered and the filtrate (B9-3) was used in the next step without purification.

### Step 4. tert-Butyl ((2S,3S)-3-aminobutan-2-yl)carbamate (B9-4)

To a solution of compound B9-3 in EtOH (600 mL) was added tert-Butyl phenyl carbonate (24 g, 121 mmol) and the resulting mixture was stirred at reflux overnight and then concentrated under reduced pressure. The crude product was purified by silica gel chromatography eluting with DCM: MeOH (10:1) to afford compound B9-4 (6 g, 26% for two steps).

### Step 5. tert-Butyl ((2S,3S)-3-((4-nitrophenyl)sulfonamido)butan-2-yl)carbamate (B9-5)

To an ice-cooled solution of B9-4 (6 g, 32 mmol) and TEA (5.3 g, 52 mmol) in DCM (350 mL) was added a solution of NsCl (7.6 g, 34 mmol) in DCM (60 mL) and the resulting yellow mixture was stirred at 0° C. and then at rt overnight. The reaction was quenched with H₂O (300 mL) and the aqueous phase was extracted with EtOAc (3×500 mL). The combined organic phases were washed with brine, dried over anhydrous sodium sulfate, filtered, and concentrated under reduced pressure. The crude product was purified by normal phase chromatography using a silica gel column eluting with PE:EA (2:1) to give the compound B9-5 (7.4 g, 64%). ¹H NMR (300 MHz, CDCl₃): δ 8.35 (d, J=8.4 Hz, 2H), 8.07 (d, J=8.4 Hz, 2H), 5.98 (br, 1H), 4.57 (br, 1H), 3.59-3.54 (m, 1H), 3.29-3.27 (m, 1H), 1.44 (s, 9H), 1.15-1.04 (m, 6H).

### Example 5.5: Synthesis of (R)-methyl 3-amino-2-(N-methyl-2-nitrophenylsulfonamido) propanoate hydrochloride (B11)

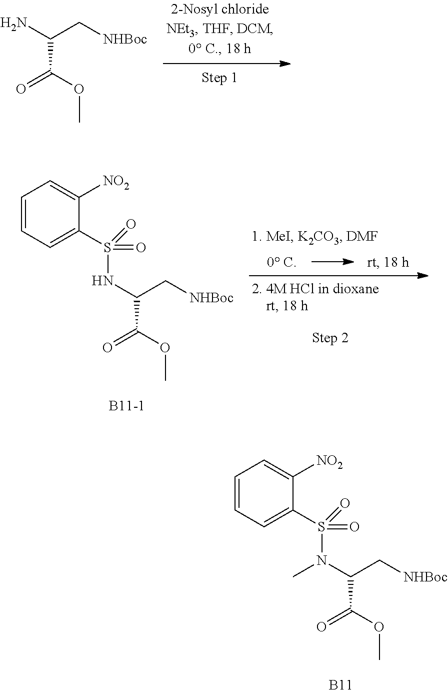

### Step 1. (R)-methyl 3-((tert-butoxycarbonyl)amino)-2-(2-nitrophenylsulfonamido)propanoate (B11-1)

To Boc-Dap-OMe hydrochloride (3.74 g, 14.68 mmol) dissolved in DCM (31 mL) was added triethylamine (4.7 mL, 33.7 mmol) and the resulting mixture was cooled to 0° C. in an ice bath. A solution of 2-nosyl chloride (3.60 g, 16.24 mmol) in THF (27 mL) was then added dropwise at 0° C. using an addition funnel and the reaction mixture was allowed to stir at 0° C. overnight and then concentrated. The obtained off-white solid was taken up in EtOAc and then transferred to a separatory funnel. The organic layer was washed with water (3×) and brine (1×), dried with MgSO₄, filtered, and concentrated in vacuo to yield B11-1 (5.56 g, 94%) as an off-white solid. Analytical method 7; t_{R} 1.01 min; [M−H]⁻=402.0.

### Step 2. (R)-methyl 3-amino-2-(N-methyl-2-nitrophenylsulfonamido)propanoate hydrochloride (B11)

To B11-1 (5.56 g, 13.78 mmol) in DMF (25 mL) under an atmosphere of nitrogen and cooled to 0° C. was added potassium carbonate (9.52 g, 68.9 mmol) in one portion. The resulting mixture was stirred at 0° C. for 30 min and Mel (2.6 mL, 41.6 mmol) was then added dropwise at 0° C. The ice bath was removed upon complete addition of methyl iodide. The reaction mixture was allowed to warm to rt and then stirred overnight. The reaction mixture was filtered and DMF was evaporated on a Genevac. The crude residue was diluted with EtOAc and transferred to a separatory funnel. The organic layer was washed with water (3×) and brine (1×), dried with MgSO₄, filtered, and concentrated in vacuo. The crude yellow oil (6.05 g, 68%) was taken up in 4 M HCl in Dioxane (17 mL, 68.0 mmol) and the resulting mixture was stirred at rt overnight. The reaction mixture was then concentrated under vacuum to yield B11 as a thick orange oil (6.92 g, 70%). Analytical method 7; t_{R}=0.48 min; [M+H]⁺=317.7.

### Example 5.6: Synthesis of tert-butyl (S)-(2-(methylamino)-6-((2-nitrophenyl)sulfonamido) hexyl)carbamate (B12)

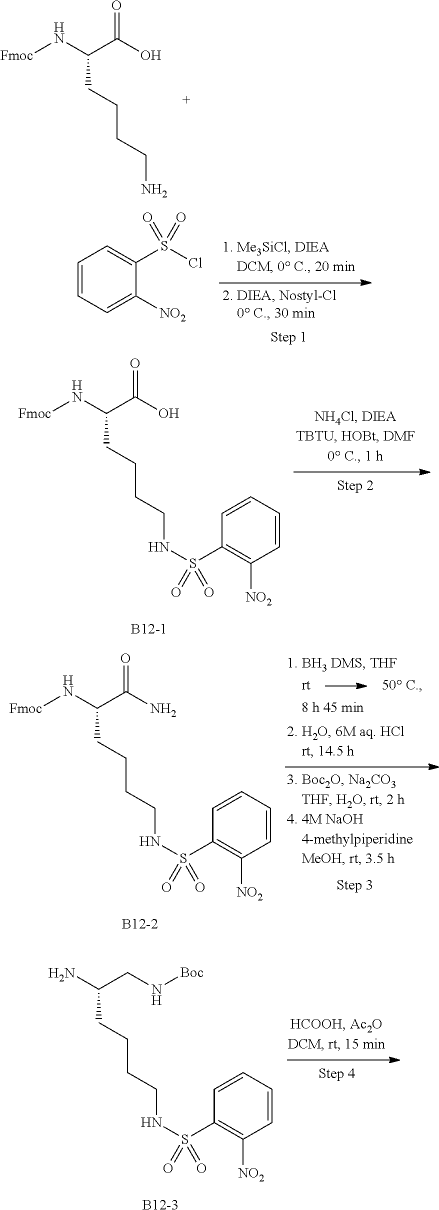

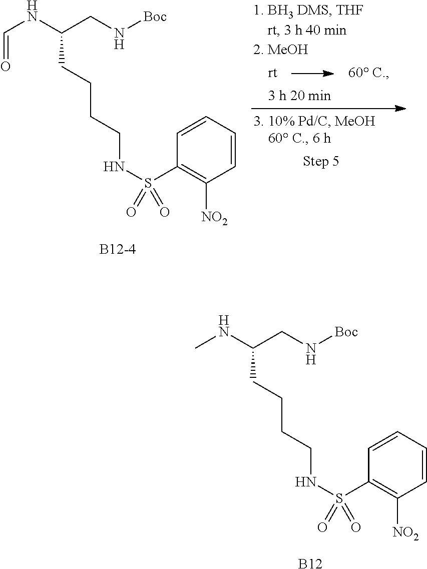

### Step 1. N²-(((9H-Fluoren-9-yl)methoxy)carbonyl)-N⁶-((2-nitrophenyl)sulfonyl)-L-lysine (B12-1)

To a suspension of Fmoc-Lys-OH HCl (4.05 g, 10 mmol) in DCM (80 mL) at 0° C. was added Me₃SiCl (3.83 mL, 30.0 mmol) and DIEA (8.73 mL, 50.0 mmol) and the resulting mixture was stirred for 20 min at 0° C. and became a clear solution. DIEA (1.747 mL, 10.00 mmol) and 2-nitrobenzene-1-sulfonyl chloride (2.327 g, 10.50 mmol) were added and the reaction mixture was stirred for 30 min at 0° C., and then concentrated to dryness in vacuo. The resulting residue was partitioned between EtOAc (150 mL) and 5% aq. KHSO₄ (50 mL). The organic layer was washed with 5% aq. KHSO₄ (3×50 mL) and brine (50 mL), dried over Na₂SO₄, filtered, and concentrated to dryness in vacuo to afford B12-1 (5.36 g, 9.68 mmol, 97% yield) as a yellow foam. The crude product was used in the next step without purification. Analytical method 10; t_{R}=1.08 min; [M+NH4]⁺=571.3.

### Step 2. (9H-Fluoren-9-yl)methyl (S)-(1-amino-6-((2-nitrophenyl)sulfonamido)-1-oxohexan-2-yl)carbamate (B12-2)

To a suspension of B12-1 (5.36 g, 9.68 mmol), NH₄Cl (1.036 g, 19.36 mmol) and HOBt (1.483 g, 9.68 mmol) in DMF (80 mL) at 0° C. was added DIEA (6.76 mL, 38.7 mmol). The resulting suspension was stirred for 5 min at 0° C., and then TBTU (3.42 g, 10.65 mmol) was added. After stirring at 0° C. for 1 h, the reaction mixture was partitioned between EtOAc (250 mL) and 5% aq. NaHCO₃ (100 mL). The organic layer was washed with 5% aq. NaHCO₃ (3×50 mL) and brine (25 mL), dried over Na₂SO₄, filtered, and concentrated to dryness in vacuo to afford B12-2 (5.56 g, assumed to be 9.66 mmol) as a yellow oil. The crude product was used in the next step without purification. Analytical method 19; t_{R}=1.04 min; [M+H]⁺=553.3.

### Step 3. tert-Butyl (S)-(2-amino-6-((2-nitrophenyl)sulfonamido)hexyl)carbamate (B12-3)

Step 3-1: To B12-2 (9.66 mmol) dissolved in THF (60 mL) was added and BH₃.DMS (5.50 mL, 58.0 mmol) and the resulting mixture was stirred for 2 h 15 min at rt, and then for 6.5 h at 50° C. The reaction mixture was allowed to cool to rt.

Step 3-2: H₂O (1 mL) and 6 M aq. HCl (2 mL) were added and the reaction mixture was stirred for 14.5 h at rt.

Step 3-3: 0.5 M aq. Na₂CO₃ (48.3 mL, 24.15 mmol) and a solution of Boc₂O (2.243 mL, 9.66 mmol) in THF (20 mL) were added. The resulting mixture was stirred for 2 h at rt, quenched with 8 M MeNH₂ in EtOH (1 mL), and stirred at rt for 30 min.

Step 3-4: 4 M aq. NaOH (9.66 mL, 38.6 mmol) was added and the reaction mixture was stirred for 85 min at rt. 4-Methylpiperidine (4 mL) was then added and the reaction mixture was stirred for 40 min at rt. Additional 4-methylpiperidine (8 mL) was added and stirring at rt was continued for 30 min. MeOH (10 mL) was then added with stirring for 25 min at rt. Additional MeOH (20 mL) and 4-methylpiperidine (10 mL) were added and stirred for 30 min. The reaction mixture was then concentrated in vacuo and the crude product was purified by flash chromatography on silica gel (eluent A: EtOAc/DIEA (98:2), eluent B: EtOAc/MeOH/DIEA (95:5:2)) Pure fractions were combined and concentrated to dryness in vacuo to afford B12-3 (1.326 g, 3.18 mmol, 33% yield) as a yellow oil. Analytical method 10; t_{R}=0.69 min; [M+H]⁺=417.2.

### Step 4. tert-Butyl (S)-(2-formamido-6-((2-nitrophenyl)sulfonamido)hexyl)carbamate (B12-4)

A mixture of formic acid (0.611 mL, 15.92 mmol) and Ac₂O (1.502 mL, 15.92 mmol) was stirred for 40 min at rt, and then added to a solution of B12-3 (1.326 g, 3.18 mmol) in DCM (15 mL). The reaction mixture was stirred for 15 min at rt, and then concentrated to dryness in vacuo. The obtained residue was partitioned between EtOAc (80 mL) and 5% aq. NaHCO₃ (10 mL) The organic layer was washed with 5% aq. NaHCO₃ (4×10 mL) and brine (10 mL), dried over Na₂SO₄, filtered, and concentrated to dryness in vacuo to afford B12-4 (1.217 g, 2.74 mmol, 86% yield) as a yellow foam. The crude product was used in the next step without purification. Analytical method 10; t_{R}=0.87 min; [M+H]⁺=445.2.

### Step 5. tert-Butyl (S)-(2-(methylamino)-6-((2-nitrophenyl)sulfonamido)hexyl)carbamate (B12)

Step 5-1: To B12-4 (1.217 g, 2.74 mmol) dissolved in THF (20 mL) was added BH₃.DMS (1.300 mL, 13.69 mmol) and the resulting mixture was stirred for 3 h 40 min at rt.

Step 5-2: The reaction was quenched by the addition of MeOH (2 mL) and the resulting solution was stirred for 125 min at rt. MeOH (3 mL) was added and stirring was continued at 60° C. for 75 min. The reaction mixture was then concentrated to dryness in vacuo.

Step 5-3: The obtained residue was dissolved in MeOH (20 mL) and a suspension of 10% Pd/C (0.087 g, 0.082 mmol) in H₂O (1 mL) was added. The resulting mixture was stirred for 3.5 h at 60° C. Additional 10% Pd/C (0.087 g, 0.082 mmol) in H₂O (1 mL) was added and stirring at 60° C. was continued for 2.5 h. The reaction mixture was filtered over Hyflo and the filtrate was concentrated to dryness in vacuo to afford B12 (1.048 g, 2.434 mmol, 89% yield) as a yellow oil. The crude product was used in the next step without purification. Analytical method 10; t_{R}=0.71 min; [M+H]⁺=431.3.

### Example 5.7: Synthesis of (S)—N-(4-(dimethylamino)-2-(methylamino)butyl)-2-nitrobenzenesulfonamide (B13)

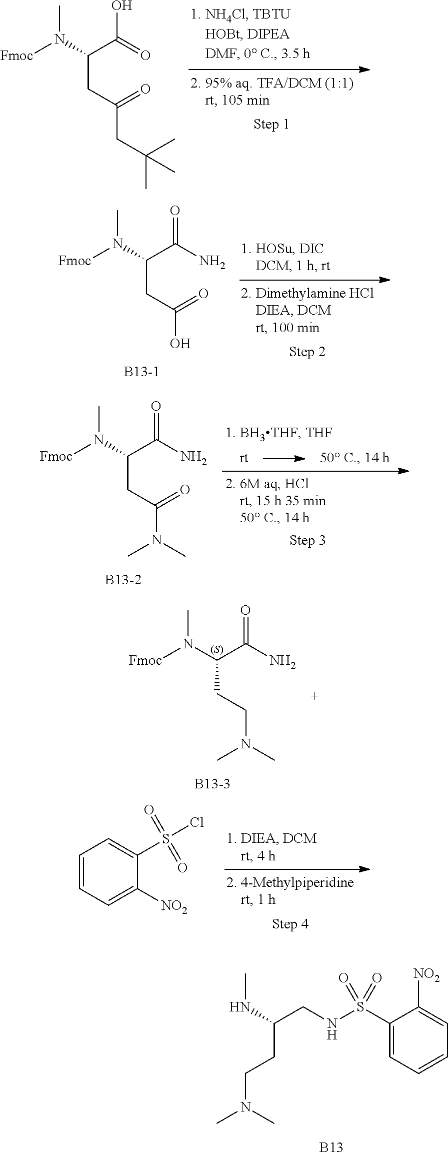

### Step 1. ((S)-3-((((9H-Fluoren-9-yl)methoxy)carbonyl)(methyl)amino)-4-amino-4-oxobutanoic acid (B13-1)

Step 1-1: To N-Fmoc-N-Me-Asp(tBu)-OH (851 mg, 2.00 mmol), NH₄Cl (0.214 g, 4.00 mmol) and HOBt (306 mg, 2.000 mmol) was added DMF (10 mL). The resulting suspension was cooled to 0° C. and DIEA (1.397 mL, 8.00 mmol) was added. The suspension was stirred for 5 min at 0° C. and TBTU (0.706 g, 2.200 mmol) was then added and stirred for 2.5 h at 0° C. Additional NH₄Cl (0.107 g, 2.000 mmol), DIEA (0.699 mL, 4.00 mmol) and TBTU (0.706 g, 2.200 mmol) were added. The reaction mixture was stirred for 1 h at 0° C. and then partitioned between EtOAc (50 mL) and 5% aq. NaHCO₃ (20 mL). The organic layer was washed with 5% aq. NaHCO₃ (3×10 mL) and brine (10 mL), dried over Na₂SO₄, filtered, and concentrated to dryness in vacuo to afford a colorless oil.

Step 1-2: The crude colorless oil was dissolved in 95% aq. TFA/DCM (1:1) (20 mL). The resulting solution was stirred for 105 min at rt and then concentrated to dryness in vacuo. The crude product was purified by flash chromatography on silica gel (eluent A: Heptane/AcOH (99:1), eluent B: EtOAc/AcOH (99:1)). Pure fractions were combined and concentrated to dryness in vacuo. The obtained residue was dissolved in DCM and the resulting solution was concentrated to dryness in vacuo. This step was repeated twice. B13-1 (691 mg, 1.876 mmol, 94% yield) was obtained as a white foam. Analytical method 10; t_{R}=0.84 min; [M+H]⁺=369.2.

### Step 2. (9H-Fluoren-9-yl)methyl (S)-(1-amino-4-(dimethylamino)-1,4-dioxobutan-2-yl)(methyl)carbamate (B13-2)

To B13-1 (690 mg, 1.873 mmol) and HOSu (216 mg, 1.873 mmol) in DCM (20 mL) was added DIC (0.292 mL, 1.873 mmol) and the resulting mixture was stirred for 1 h at rt. A solution of dimethylamine hydrochloride (160 mg, 1.967 mmol) and DIEA (0.393 mL, 2.248 mmol) in DCM (20 mL) was then added dropwise over 40 min. The reaction mixture was stirred for 35 min at rt, then further dimethylamine hydrochloride (16.04 mg, 0.197 mmol) and DIEA (0.039 mL, 0.225 mmol) were added. The reaction mixture was stirred for 25 min at rt, and then concentrated to dryness in vacuo. The obtained residue was partitioned between EtOAc (60 mL) and 5% aq. NaHCO₃ (10 mL). The organic layer was washed with 5% aq. NaHCO₃ (3×10 mL) and brine (10 mL), dried over Na₂SO₄, filtered, and concentrated to dryness in vacuo. The crude product was purified by flash chromatography on silica gel (eluent A: EtOAc, eluent B: EtOAc/MeOH (90:10)). Pure fractions were combined and concentrated to dryness in vacuo. The residue was dissolved in DCM and the resulting solution was concentrated to dryness in vacuo. B13-2 (501.5 mg, 1.268 mmol, 67.7% yield) was obtained as a white foam. Analytical method 10; t_{R}=0.89 min; [M+H]⁺=396.3.

### Step 3. (9H-Fluoren-9-yl)methyl (S)-(1-amino-4-(dimethylamino)butan-2-yl)(methyl)carbamate trifluoro-acetate (B13-3)

Step 3-1: To a solution of B13-2 (500 mg, 1.264 mmol) in THF (dry) (15 mL) was added 1 M BH₃.THF in THF (10.12 mL, 10.12 mmol). The reaction mixture was stirred for 30 min at rt and then for 13.5 h at 50° C.

Step 3-2: The reaction mixture was cooled to rt, then quenched by the addition of 6 M aq. HCl (5 mL), and stirred for 35 min at rt, 9 h at 50° C., 15 h at rt, and 5 h at 50° C. The mixture was concentrated to dryness in vacuo. The obtained residue was partitioned between EtOAc (50 mL) and 4 M aq. NaOH (10 mL). The organic layer was washed with 1 M aq. NaOH (2×10 mL) and brine (10 mL), dried over Na₂SO₄, filtered, and concentrated to dryness in vacuo. The residue was dissolved in THF (5 mL) and 4 M aq. HCl (3 mL) and concentrated to dryness in vacuo to yield the HCl salt of the crude product. The crude product was purified by reversed-phase chromatography (eluent A: 0.1% TFA in H₂O and eluent B: 0.1% TFA in ACN). Pure fractions were combined and lyophilized. The residue was dissolved in DCM and the solution was concentrated to dryness in vacuo to afford the TFA salt of B13-3 (212 mg, 0.356 mmol, 28.2% yield) as a white foam. Analytical method 10; t_{R}=0.55 min; [M+H]⁺=368.3.

### Step 4. (S)—N-(4-(dimethylamino)-2-(methylamino)butyl)-2-nitrobenzenesulfonamide TFA (B13)

Step 4-1: B13-3 (210 mg, 0.353 mmol) was dissolved in DMA (3 mL). DIEA (0.185 mL, 1.058 mmol) and 2-nitrobenzene-1-sulfonyl chloride (78 mg, 0.353 mmol) were added and the resulting mixture was stirred for 4 h at rt.

Step 4-2: 4-Methylpiperidine (0.3 mL) was added and the reaction mixture was stirred for 1 h at rt and then quenched by the addition of AcOH (0.4 mL). The product was isolated by preparative reversed-phase HPLC (eluent A: 0.1% TFA in H₂O and eluent B: ACN). Pure fractions were combined and lyophilized to afford the TFA salt of B13 (113 mg, 0.202 mmol, 57.4% yield) as a colorless sticky oil. Analytical method 11; t_{R}=0.56 min; [M+H]⁺=331.1.

### Example 5.8: Synthesis of (S)—N-(4-fluoro-2-(methylamino)butyl)-2-nitrobenzenesulfonamide (B14)

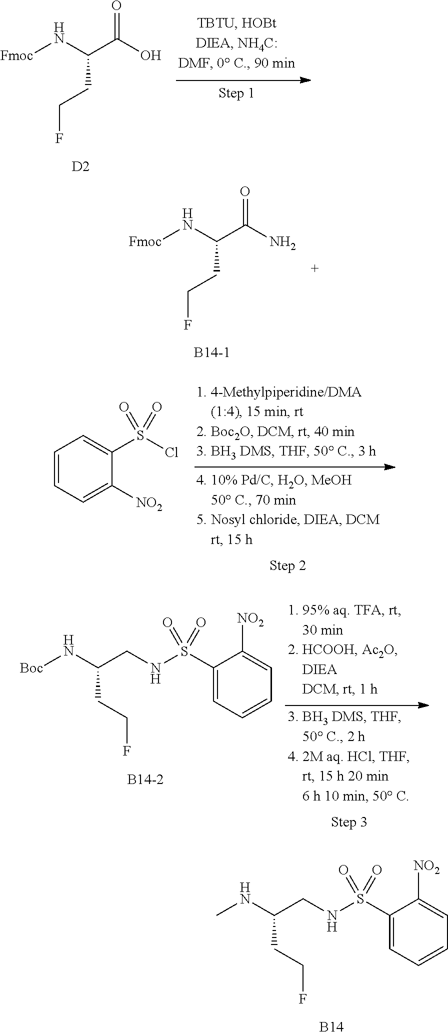

### Step 1. (9H-Fluoren-9-yl)methyl (S)-(1-amino-4-fluoro-1-oxobutan-2-yl)carbamate (B14-1)

To D2 (1.030 g, 3.0 mmol), HOBt (0.459 g, 3.00 mmol) and TBTU (1.060 g, 3.30 mmol) was added DMF (30 mL). the resulting mixture was cooled to 0° C. DIEA (0.524 mL, 3.00 mmol) was added and stirred for 5 min. Ammonium chloride (0.642 g, 12.00 mmol) and DIEA (1.572 mL, 9.00 mmol) were added. The reaction mixture was stirred for 55 min at 0° C., additional TBTU (0.193 g, 0.600 mmol) was added, and stirring at 0° C. was continued for 35 min. The reaction mixture was partitioned between EtOAc (100 mL) and 5% aq. NaHCO₃ (50 mL). The organic layer was washed with 5% aq. NaHCO₃ (3×25 mL) and brine (20 mL), dried over Na₂SO₄, filtered, and concentrated to dryness in vacuo to afford B14-1 (assumed to be 3.0 mmol) as a beige solid. The crude product was used in the next step without purification. Analytical method 10; t_{R}=0.91 min; [M+H]⁺=343.3.

### Step 2. (tert-Butyl (S)-(4-fluoro-1-((2-nitrophenyl)sulfonamido)butan-2-yl)carbamate (B14-2)

Step 2-1: B14-1 (3.0 mmol) was dissolved in 4-methylpiperidine/DMA (1:4) (20 mL). The reaction mixture was stirred for 15 min at rt and then concentrated to dryness in vacuo.

Step 2-2: To the obtained residue dissolved in DCM (20 mL) was added a solution of Boc₂O (0.697 mL, 3.00 mmol) in DCM (10 mL). The reaction mixture was stirred for 40 min at rt and then concentrated to dryness in vacuo.

Step 2-3: To a solution of the resulting residue in THF (25 mL) at 50° C. was added BH₃.DMS (1.424 mL, 15.00 mmol). The reaction mixture was stirred for 3 h at 50° C., quenched by addition of MeOH (5 mL), and then concentrated to dryness in vacuo.

Step 2-4: To the residue from Step 2-3 dissolved in MeOH (30 mL) was added a suspension of 10% Pd/C (96 mg, 0.090 mmol) in H₂O (1 mL). The reaction mixture was stirred for 70 min at 50° C., filtered over Hyflo, and concentrated to dryness in vacuo.

Step 2-5: To the residue from Step 2-4 dissolved in DCM (30 mL) was added DIEA (1.048 mL, 6.00 mmol). 2-Nitrobenzene-1-sulfonyl chloride (665 mg, 3.00 mmol) and the resulting mixture was stirred for 15 h at rt. DCM was then removed in vacuo and the resulting residue was partitioned between EtOAc (50 mL) and 5% aq. NaHCO₃ (20 mL). The organic layer was washed with 5% aq. NaHCO₃ (2×20 mL), 5% aq. KHSO₄ (3×20 mL) and brine (10 mL), dried over Na₂SO₄, filtered, and concentrated to dryness in vacuo. The crude product was purified by flash chromatography on silica gel (eluent A: heptane, eluent B: EtOAc). Pure fractions were combined and concentrated to dryness in vacuo to afford B14-2 (305 mg, 0.779 mmol, 26.0% yield for 2 steps) as a yellow foam. Analytical method 10; t_{R}=0.97 min; [M+NH4]+=409.3.

### Step 3. ((S)—N-(4-Fluoro-2-(methylamino)butyl)-2-nitrobenzenesulfonamide (B14)

Step 3-1: B14-2 (305 mg, 0.779 mmol) was dissolved in 95% aq. TFA (10 mL). The resulting solution was stirred for 30 min at rt and then concentrated to dryness in vacuo.

Step 3-2: A mixture of formic acid (0.149 mL, 3.90 mmol) and Ac₂O (0.368 mL, 3.90 mmol) was stirred for 30 min at rt and then added to a solution of the residue from Step 3-1 in DCM (10 ml). The resulting mixture was stirred for 1 h at rt and then concentrated to dryness in vacuo. The obtained residue was partitioned between EtOAc (50 mL) and 5% aq. NaHCO₃ (10 mL) The organic layer was washed with 5% aq. NaHCO₃ (3×10 mL) and brine (10 mL), dried over Na₂SO₄, filtered, and concentrated to dryness in vacuo.

Step 3-3: To a solution of the residue from Step 3-2 in THF (10 mL) at 50° C. was added BH₃.DMS (0.370 mL, 3.90 mmol). The resulting was stirred for 2 h at 50° C., quenched by addition of MeOH (2 mL), and concentrated to dryness in vacuo.

Step 3-4: The residue from 3-3 was dissolved in THF (10 mL) and 2 M aq. HCl (4 mL). The resulting solution was stirred for 15 h 20 min at rt and then for 6 h 10 min at 50° C. The THF was removed in vacuo and the obtained d residue was partitioned between EtOAc (50 mL) and 5% aq. Na₂CO₃ (10 mL). The organic layer was washed with 5% aq. Na₂CO₃ (10 mL). The aqueous layer was extracted with EtOAc (2×20 mL). The combined organic layers were washed with brine (10 mL), dried over Na₂SO₄, filtered, and concentrated to dryness in vacuo to afford B14 (209 mg, 0.685 mmol, 88% yield) as a yellow oil. The crude product was used in the next step without purification. Analytical method 10; t_{R}=0.45 min; [M+H]⁺=306.2.

### Example 5.9: Synthesis of tert-butyl (S)-(2-(methylamino)propyl)carbamate (B15)

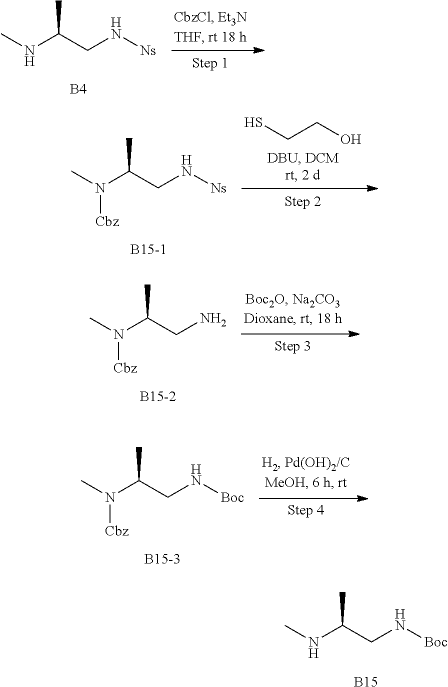

### Step 1. Benzyl (S)-methyl(1-((4-nitrophenyl)sulfonamido)propan-2-yl)carbamate (B15-1)

To a solution of B4 (7 g, 22.60 mmol) in THF (100 mL) at 0° C., was added triethylamine (9.45 ml, 67.8 mmol) and the resulting mixture was stirred at 0° C. for 30 min. Cbz chloride (6.49 g, 36.2 mmol) in THF (5 mL) was added dropwise at 0° C. and the reaction mixture was stirred at 22° C. for 18 hr. Water (5 mL) was added and the reaction mixture was concentrated under reduced pressure. 80 mL water and 20 mL saturated NaHCO₃ were added the crude residue and resulting mixture was extracted with 400 mL EtOAc. The organic layer was washed with brine, dried over Na₂SO₄, filtered, and concentrated in vacuo. The crude product was purified by normal phase chromatography using a silica gel column eluting with EtOAc/heptane 10:90 to 60:40 to afford the desired intermediate B15-1 (7.2 g, 15.90 mmol, 70.4% yield). Analytical method 7; t_{R}=1.42 min; [M+H]⁺=408.2.

### Step 2. Benzyl (S)-(1-aminopropan-2-yl)(methyl)carbamate (B15-2)

To a solution of B15-1 (7 g, 17.18 mmol) in DCM (100 mL), was added DBU (11.40 mL, 75.6 mmol) and mercapto ethanol (5.36 g, 68.8 mmol) at rt and the resulting mixture was stirred at 22° C. for 2 days. The reaction mixture was then poured into 100 mL of water. 20 mL of aq. saturated NaHCO₃ and 300 mL of EtOAc were added. The organic layer was washed with brine, dried over Na₂SO₄, filtered, and concentrated in vacuo to afford the desired intermediate B15-2 (3.82 g, 17.18 mmol, 100% yield). Analytical method 5; t_{R}=0.97 min; [M+H]⁺=223.2.

### Step 3. Benzyl (S)-(1-((tert-butoxycarbonyl)amino)propan-2-yl)(methyl)carbamate (B15-3)

To a solution of B15-2 (3.82 g, 17.19 mmol) in dioxane (60 mL) stirred at 0° C. was added aq. 1.0 M sodium carbonate (34.4 mL, 34.4 mmol) at 0° C. and the resulting mixture was stirred at 0° C. for 30 min. Boc₂O (3.75 g, 17.19 mmol) in dioxane (5 mL) was then added dropwise at 0° C. and the reaction mixture was stirred at 22° C. for 18 h. The dioxane was removed under reduced pressure and the aqueous phase was extracted with 200 mL EtOAc. The organic layer washed with brine and dried over Na₂SO₄, filtered, concentrated, and purified by normal phase chromatography using a silica gel column eluting with EtOAc/Heptane 5:95 to 60:40 to afford B15-3 (4.0 g, 72.2% yield) as a solid. Analytical method 7; t_{R}=1.43 min; [M+H]⁺=323.3. ¹H NMR (400 MHz, DMSO-d₆) b ppm 7.36 (s, 5H), 6.81-6.86 (m, 1H), 5.02-5.06 (m, 2H), 4.06-4.10 (m, 1H), 2.92-2.98 (m, 2H), 2.66-2.70 (m, 3H), 1.45 (s, 9H), 1.02-1.06 (d, 3H).

### Step 4. tert-Butyl (S)-(2-(methylamino)propyl)carbamate (B15)

B15-3 (1.50 g, 4.65 mmol) was dissolved in MeOH (10 mL) at 22° C. The resulting mixture was stirred and N₂ was bubbled through the solution at rt for 5 min. 20% Palladium hydroxide on carbon (0.327 g, 0.465 mmol) was then added and the resulting mixture was stirred at rt for 6 h. The reaction mixture was filtered through a pad of Celite® and the pad was washed with MeOH and EtOAc. The filtrate was concentrated in vacuo to afford the desired intermediate B15. Analytical method 7; t_{R}=0.47 min; [M+H]⁺=189.2. ¹H NMR (400 MHz, CD₃OD) b ppm 5.03-5.08 (m, 1H), 3.16-3.22 (m, 1H), 3.06-3.10 (m, 1H), 3.02-3.06 (m, 1H), 2.72-2.76 (m, 1H), 2.44 (s, 3H), 1.45 (s, 9H), 1.02-1.06 (d, 3H).

### Example 6: Building Block AB—Polymer-Bound Dimers

### Example 6.1: Synthesis of PS-(2-Chlorotrityl) methyl (R)-4-(((S)-2-aminopropyl)(methyl) amino)-3-benzyl-4-oxobutanoate (AB3)

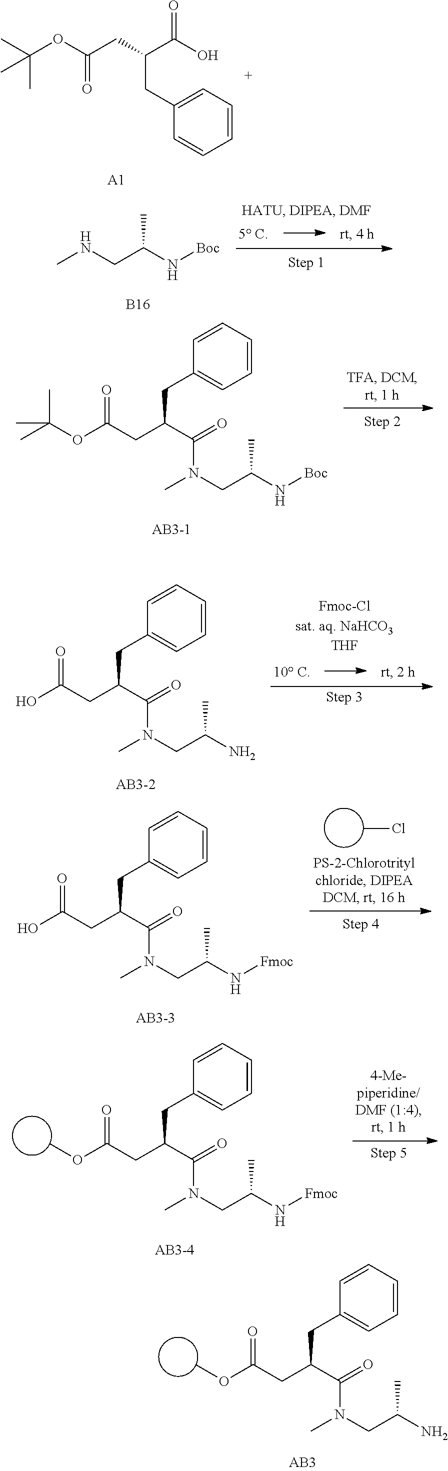

### Step 1. tert-Butyl (R)-3-benzyl-4-(((S)-2-((tert-butoxycarbonyl)amino)propyl)(methyl)amino)-4-oxobutanoate (AB3-1)

To a cold stirred solution of A1 (211 mg, 0.797 mmol) and B16 (150 mg, 0.797 mmol) in DMF (5 mL) was added DIPEA (0.557 mL, 3.19 mmol) followed by HATU (303 mg, 0.797 mmol) at 5-10° C. The resulting mixture was removed from the bath and stirred at rt for 4 h. The reaction mixture was poured in ice-cold water (5 mL) and the product was extracted with ethyl acetate (2×20 mL). The combined organic layers were washed with 1 M HCl (20 mL), 1 M NaOH (20 mL), water (20 mL) and brine solution (20 mL), dried over anhydrous Na₂SO₄ and concentrated under vacuum to afford crude AB3-1 (300 mg, 87%). The crude compound was used in the next step without purification. Analytical method 7; t_{R}=1.18 min; [M+H]⁺=435.5.

### Step 2. (R)-4-(((S)-2-Aminopropyl)(methyl)amino)-3-benzyl-4-oxobutanoic acid (AB3-2)

AB3-1 (300 mg, 0.690 mmol) in 20% TFA in DCM (10 mL) at rt was stirred for 1 h and the resulting mixture was then concentrated in vacuo. The crude oil was taken up in DCM (10 mL) and concentrated down again. This was repeated with fresh DCM (2×) to yield AB3-2 (271 mg, 100%) as a clear oil which was used as is directly in the next step without purification. Analytical method 7; t_{R}=0.44 min; [M+H]⁺=279.3.

### Step 3. (R)-4-(((S)-2-((((9H-Fluoren-9-yl)methoxy)carbonyl)amino)propyl)(methyl)amino)-3-benzyl-4-oxobutanoic acid (AB3-3)

To a cold stirred solution of AB3-2 (271 mg, 0.69 mmol) in THF (30 mL) was added Fmoc-Cl (179 mg, 0.69 mmol) portion wise over 30 minutes at 10° C., followed by sat. sodium bicarbonate solution (2.0 mL) over a period of 30 minutes at the same temperature. The resulting mixture was stirred at room temperature for 2 h and diluted with water (50 mL). The Aqueous phase was extracted with ethyl acetate (2×100 mL). The combined organic layers were washed with brine solution (200 mL), dried over anhydrous Na₂SO₄ and concentrated under vacuum to afford crude compound. The crude material was purified by 230-400-mesh silica gel column chromatography eluting with 20% ethyl acetate in petroleum ether to afford compound AB3-3 (210 mg, 61%) as a yellow gum solid. Analytical method 7; t_{R}=1.11 min; [M+H]⁺=501.3.

### Step 4. PS-(2-Chlorotrityl)-(R)-4-(((S)-2-((((9H-fluoren-9-yl)methoxy)carbonyl)amino)propyl) (methyl)-amino)-3-benzyl-4-oxobutanoic acid (AB3-4)

2-Chlorotrityl chloride resin (336 mg, 0.336 mmol) was pre-washed with DCM (3×20 mL). AB3-3 (210 mg, 0.420 mmol) in DCM (10 mL) and DIPEA (0.220 mL, 1.259 mmol) were added to the resin. The resulting mixture was shaken at rt for 16 h, washed with DCM (3×20 mL), and then shaken in DCM/MeOH (5:2) (7 mL) to cap the resin for 30 min. The resin was then filtered, washed with DMF (2×20 mL) and DCM (2×20 mL), and dried under vacuum. This provided resin AB3-4 (266 mg, crude), which was carried onto the next step without purification.

### Step 5. PS-(2-Chlorotrityl)-(R)-4-(((S)-2-aminopropyl)(methyl)amino)-3-benzyl-4-oxobutanoic acid (AB3)

To AB3-4 (266 mg, 0.336 mmol) was added 20% 4-methylpiperidine in DMF (10 mL) and the resulting mixture was shaken at rt for 2 h. The resin was then filtered, washed with DMF (2×10 mL) and DCM (2×10 mL), and dried under vacuum. This provided resin AB3 (191 mg, crude), which was carried onto the next step without purification.

The polymer bound Building Blocks AB (BB AB) in Table 20 below were prepared according to the procedure used for AB3 (Example 6.1), AB1 (Example 6.2), AB5 (Example 6.3), or AB6-3 (Example 6.4) from the respective intermediates shown in Table 17 and Table 19.

TABLE 20
Building block AB - Polymer-bound dimers

| BB | | | |
|---|---|---|---|
| No. | Structure | Compound Name | Synthesis Protocol |
| AB2 | 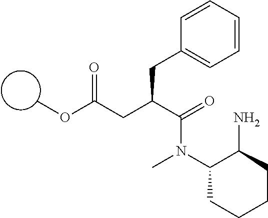 [SMILES: COC(=O)C[C@@H](Cc1ccccc1)C(=O)N(C)[C@H]1CCCC[C@@H]1N] | PS-(2-Chlorotrityl) (R)-4-(((1S,2S)-2- aminocyclohexyl) (methyl)amino)-3- benzyl-4- oxobutanoate | AB3 (Example 6.1) Example 6.2 |
| AB4 | 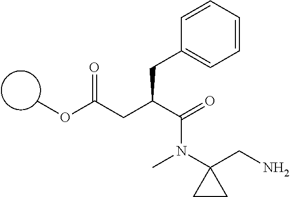 [SMILES: COC(=O)C[C@@H](Cc1ccccc1)C(=O)N(C)C1(CN)CC1] | PS-(2-Chlorotrityl) (R)-4-((1-(amino- methyl)cyclopropyl) (methyl)amino)- 3-benzyl-4- oxobutanoate | AB1 (Example 6.2)- starting from AB4-1 (see Example 6.6). |
| AB5 | 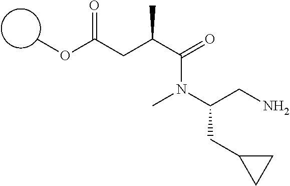 [SMILES: COC(=O)C[C@@H](C)C(=O)N(C)[C@H](CN)CC1CC1] | PS-(2-Chlorotrityl) (R)-4-(((S)-1-amino- 3-cyclopropylpropan- 2-yl)(methyl)amino)- 3-methyl-4- oxobutanoate | AB5 (Example 6.3) |
| AB6 | 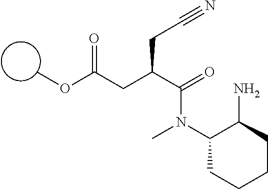 [SMILES: COC(=O)C[C@@H](CC#N)C(=O)N(C)[C@H]1CCCC[C@@H]1N] | PS-(2-Chlorotrityl) (R)-4-(((1S,2S)-2- aminocyclohexyl) (methyl)amino)-3- (cyanomethyl)-4- oxobutanoate | AB3 (Example 6.1)-starting from AB6-3 (see Example 6.4) |
| AB7 | 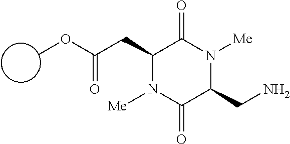 [SMILES: COC(=O)C[C@H]1C(=O)N(C)[C@@H](CN)C(=O)N1C] | PS-(2-Chlorotrityl) 2- ((2S,5S)-5- (aminomethyl)-1,4- dimethyl-3,6- dioxopiperazin-2- yl)acetate | AB3 (Example 6.1)-starting from AB7-4 (see Example 6.5). |
| AB8 | 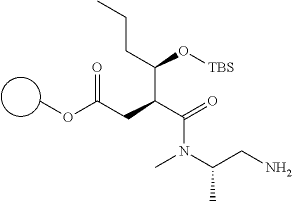 | PS-(2-Chlorotrityl) (3S,4R)-3-(((S)-1- aminopropan-2- yl)(methyl)carba- moyl)-4-((tert- butyldimethylsilyl) oxy)heptanoate | AB5 (Example 6.3)-starting from AB8-6 as starting material (see Example 6.7). |
| AB9 | 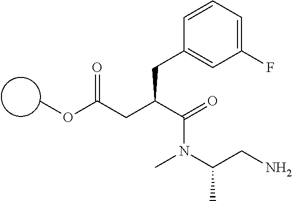 [SMILES: COC(=O)C[C@@H](Cc1cccc(F)c1)C(=O)N(C)[C@@H](C)CN] | PS-(2-Chlorotrityl) (R)-4-(((S)-1- aminopropan-2- yl)(methyl)amino)-3- (3-fluorobenzyl)-4- oxobutanoate | AB5 (Example 6.3)-starting from A19 and B4. |
| AB10 | 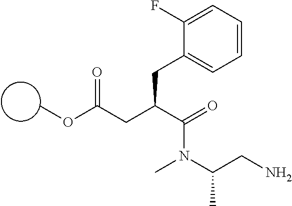 [SMILES: COC(=O)C[C@@H](Cc1ccccc1F)C(=O)N(C)[C@@H](C)CN] | PS-(2-Chlorotrityl) (R)-4-(((S)-1- aminopropan-2- yl)(methyl)amino)-3- (2-fluorobenzyl)-4- oxobutanoate | AB5 (Example 6.3)-starting from A20 and B4. |
| AB11 | 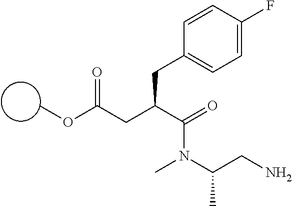 [SMILES: COC(=O)C[C@@H](Cc1ccc(F)cc1)C(=O)N(C)[C@@H](C)CN] | PS-(2-Chlorotrityl) (R)-4-(((S)-1- aminopropan-2- yl)(methyl)amino)-3- (4-fluorobenzyl)-4- oxobutanoate | AB5 (Example 6.3)-starting from A21 and B4. |
| AB12 | 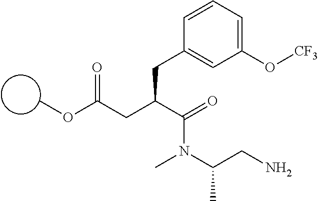 [SMILES: COC(=O)C[C@@H](Cc1cccc(OC(F)(F)F)c1)C(=O)N(C)[C@@H](C)CN] | PS-(2-Chlorotrityl) (R)-4-(((S)-1- aminopropan-2- yl)(methyl)amino)-4- oxo-3-(3- (trifluoromethoxy) benzyl)butanoate | AB5 (Example 6.3)-starting from A22 and B4. |
| AB13 | 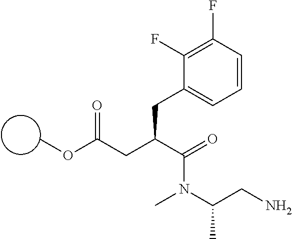 [SMILES: COC(=O)C[C@@H](Cc1cccc(F)c1F)C(=O)N(C)[C@@H](C)CN] | PS-(2-Chlorotrityl) (R)-4-(((S)-1- aminopropan-2- yl)(methyl)amino)-3- (2,3-difluorobenzyl)- 4-oxobutanoate | AB5 (Example 6.3)-starting from A23 and B4. |
| AB14 | 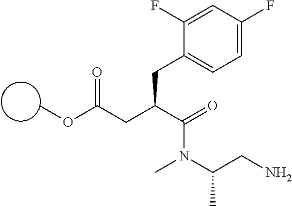 [SMILES: COC(=O)C[C@@H](Cc1ccc(F)cc1F)C(=O)N(C)[C@@H](C)CN] | PS-(2-Chlorotrityl) (R)-4-(((S)-1- aminopropan-2- yl)(methyl)amino)-3- (2,4-difluorobenzyl)- 4-oxobutanoate | AB5 (Example 6.3)-starting from A24 and B4. |
| AB15 | 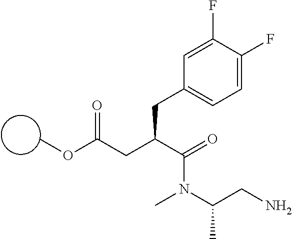 [SMILES: COC(=O)C[C@@H](Cc1ccc(F)c(F)c1)C(=O)N(C)[C@@H](C)CN] | PS-(2-Chlorotrityl) (R)-4-(((S)-1- aminopropan-2- yl)(methyl)amino)-3- (3,4-difluorobenzyl)- 4-oxobutanoate | AB5 (Example 6.3)-starting from A25 and B4. |
| AB16 | 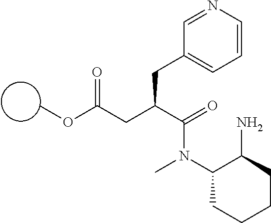 [SMILES: COC(=O)C[C@@H](Cc1cccnc1)C(=O)N(C)[C@H]1CCCC[C@@H]1N] | PS-(2-Chlorotrityl) (R)-4-(((1S,2S)-2- aminocyclohexyl)- (methyl)amino)-4- oxo-3-(pyridin-3- ylmethyl)butanoate | AB3 (Example 6.1)-starting from A2 and B1. |
| AB17 | 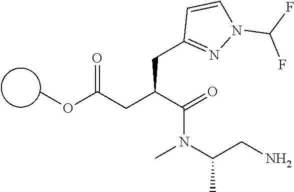 [SMILES: COC(=O)C[C@@H](Cc1ccn(C(F)F)n1)C(=O)N(C)[C@@H](C)CN] | PS-(2-Chlorotrityl) 4- (((S)-1- aminopropan-2- yl)(methyl)amino)-3- ((1-(difluoromethyl)- 1H-pyrazol-3- yl)methyl)-4- oxobutanoate | AB5 (Example 6.3)-starting from A26 and B4. |
| AB18 | 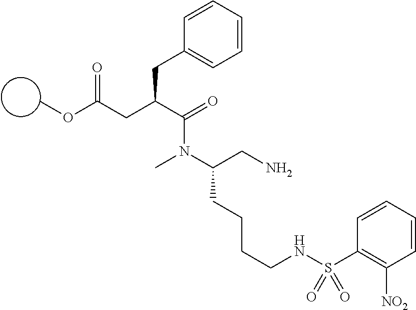 [SMILES: COC(=O)C[C@@H](Cc1ccccc1)C(=O)N(C)[C@H](CN)CCCCNS(=O)(=O)c1ccccc1[N+](=O)[O-]] | PS-(2-Chlorotrityl) (R)-4-(((S)-1-amino- 6-((2-nitrophenyl) sulfonamido)hexan- 2-yl)(methyl)amino)- 3-benzyl-4- oxobutanoate | AB3 (Example 6.1)-starting from A1 and B12. |
| AB19 | 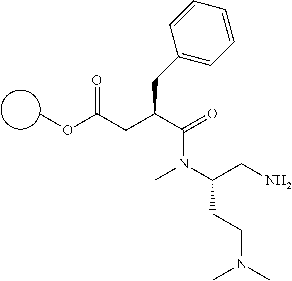 | PS-(2-Chlorotrityl) (R)-4-(((S)-1-amino- 4-(dimethylamino) butan-2- yl)(methyl)amino)-3- benzyl-4- oxobutanoate | AB5 (Example 6.3)-starting from A1 and B13. |
| AB20 | 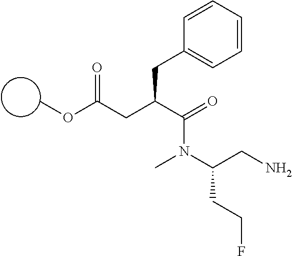 [SMILES: COC(=O)C[C@@H](Cc1ccccc1)C(=O)N(C)[C@H](CN)CCF] | PS-(2-Chlorotrityl) (R)-4-(((S)-1-amino- 4-fluorobutan-2- yl)(methyl)amino)-3- benzyl-4- oxobutanoate | AB5 (Example 6.3)-starting from A1 and B14. |
| AB21 | 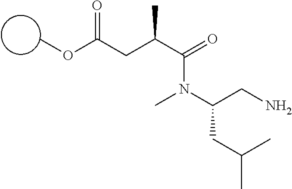 [SMILES: COC(=O)C[C@@H](C)C(=O)N(C)[C@H](CN)CC(C)C] | PS-(2-Chlorotrityl) (R)-4-(((S)-1-amino- 4-methylpentan-2- yl)(methyl)amino)-3- methyl-4- oxobutanoate | AB5 (Example 6.3)-starting from A29 and B6. |
| AB22 | 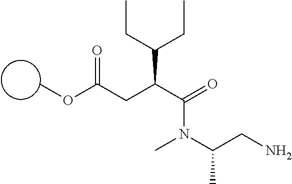 [SMILES: CCC(CC)[C@H](CC(=O)OC)C(=O)N(C)[C@@H](C)CN] | PS-(2-Chlorotrityl) (S)-3-(((S)-1-amino- propan-2-yl)(methyl) carbamoyl)-4-ethyl- hexanoate | AB5 (Example 6.3)-starting from A7 and B4. |
| AB23 | 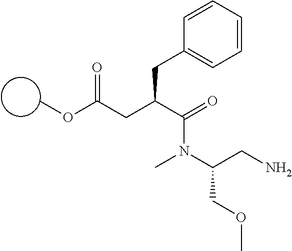 [SMILES: COC[C@@H](CN)N(C)C(=O)[C@@H](CC(=O)OC)Cc1ccccc1] | PS-(2-Chlorotrityl) (R)-4-(((R)-1-amino- 3-methoxypropan-2- yl)(methyl)amino)-3- benzyl-4- oxobutanoate | AB5 (Example 6.3)-starting from A1 and B18. |
| AB24 | 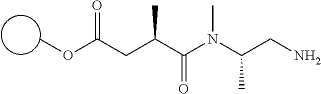 | PS-(2-Chlorotrityl) (R)-4-(((S)-1- aminopropan-2- yl)(methyl)amino)-3- methyl-4- oxobutanoate | AB1 (Example 6.2)-starting from AB29 and B4 0 |
| AB25 | 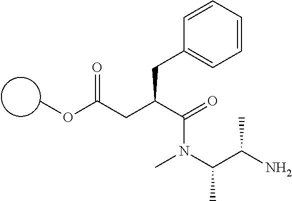 [SMILES: COC(=O)C[C@@H](Cc1ccccc1)C(=O)N(C)[C@@H](C)[C@H](C)N] | PS-(2-Chlorotrityl) (R)-4-(((2S,3S)-3- aminobutan-2- yl)(methyl)amino)-3- benzyl-4- oxobutanoate | AB3 (Example 6.1)-starting from A1 and B9. |
| AB26 | 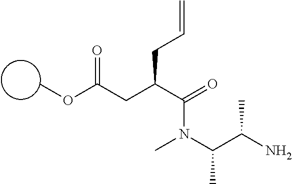 [SMILES: C=CC[C@H](CC(=O)OC)C(=O)N(C)[C@@H](C)[C@H](C)N] | PS-(2-Chlorotrityl) (R)-3-(((2S,3S)-3- aminobutan-2-yl) (methyl)carbamoyl) hex-5-enoate | AB3 (Example 6.1)-starting from A5 and B9. |
| AB27 | 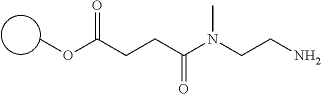 | PS-(2-Chlorotrityl) 4- ((2-aminoethyl) (methyl)amino)-4- oxobutanoate | AB3 (Example 6.1)-starting from A33 and B19. After Steps 1 to 3, an additional step for acidic hydrolysis of the methyl ester (aq. HCl, dioxane, 100° C.) was |
| | | | required. The final two |
| | | | steps were carried out as |
| | | | described for AB3. |
| AB28 | 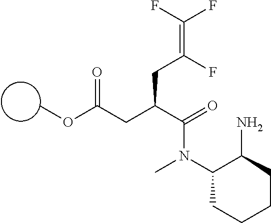 [SMILES: COC(=O)C[C@@H](CC(F)=C(F)F)C(=O)N(C)[C@H]1CCCC[C@@H]1N] | PS-(2-Chlorotrityl) (S)-3-(((1S,2S)-2- aminocyclohexyl) (methyl)carbamoyl)- 5,6,6-trifluorohex-5- enoate | AB3 (Example 6.1)-starting from A3 and B1. |
| AB29 | 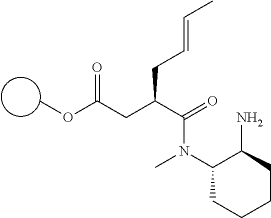 [SMILES: C/C=C/C[C@H](CC(=O)OC)C(=O)N(C)[C@H]1CCCC[C@@H]1N] | PS-(2-Chlorotrityl) (R,E)-3-(((1S,2S)-2- aminocyclohexyl) (methyl)carbamoyl) hept-5-enoate | AB3 (Example 6.1)-starting from A6 and B1. |
| AB30 | 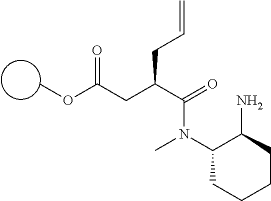 [SMILES: C=CC[C@H](CC(=O)OC)C(=O)N(C)[C@H]1CCCC[C@@H]1N] | PS-(2-Chlorotrityl) (R)-3-(((1S,2S)-2- aminocyclohexyl) (methyl)carbamoyl) hex-5-enoate | AB3 (Example 6.1)-starting from A5 and B1. |
| AB31 | 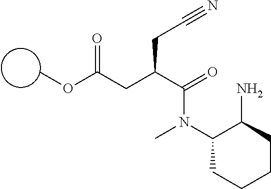 [SMILES: COC(=O)C[C@@H](CC#N)C(=O)N(C)[C@H]1CCCC[C@@H]1N] | PS-(2-Chlorotrityl) (R)-4-(((1S,2S)-2- aminocyclohexyl) (methyl)amino)-3- (cyanomethyl)-4- oxobutanoate | AB6-3 (Example 6.4) followed by deprotection as described in Example 6.2, Steps 4 to 6. |
| AB32 | 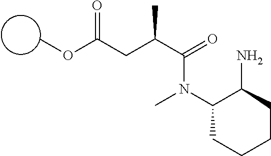 [SMILES: COC(=O)C[C@@H](C)C(=O)N(C)[C@H]1CCCC[C@@H]1N] | PS-(2-Chlorotrityl) (R)-4-(((1S,2S)-2- aminocyclohexyl) (methyl)amino)-3- methyl-4- oxobutanoate | AB3 (Example 6.1)-starting from A29 and B1. |
| AB33 | 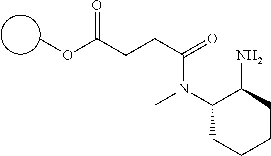 [SMILES: COC(=O)CCC(=O)N(C)[C@H]1CCCC[C@@H]1N] | PS-(2-Chlorotrityl) 4- (((1S,2S)-2- aminocyclohexyl) (methyl)amino)-4- oxobutanoate | AB3 (Example 6.1)-starting from A34 and B1. |
| AB34 | 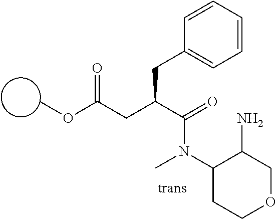 [SMILES: COC(=O)C[C@@H](Cc1ccccc1)C(=O)N(C)C1CCOCC1N] | PS-(2-Chlorotrityl) (R)-4-(((3S*,4R*)-3- aminotetrahydro-2H- pyran-4- yl)(methyl)amino)-3- benzyl-4- oxobutanoate | AB3 (Example 6.1)-starting from A1 and B3. |
| AB35 | 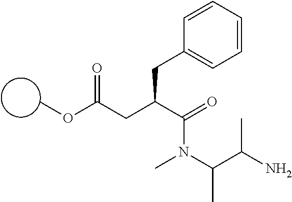 [SMILES: COC(=O)C[C@@H](Cc1ccccc1)C(=O)N(C)C(C)C(C)N] | PS-(2-Chlorotrityl) (3R)-4-((3- aminobutan-2- yl)(methyl)amino)-3- benzyl-4- oxobutanoate | AB3 (Example 6.1)-starting from A1 and B10. |
| AB36 | 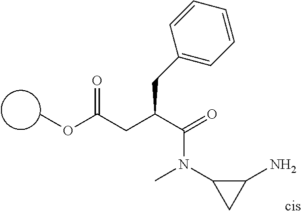 [SMILES: COC(=O)C[C@@H](Cc1ccccc1)C(=O)N(C)C1CC1N] | PS-(2-Chlorotrityl) (R)-4-(((1R*,2S*)- 2-aminocyclo- propyl)(methyl) amino)-3-benzyl-4- oxobutanoate | AB3 (Example 6.1)-starting from A1 and B2. |
| AB37 | 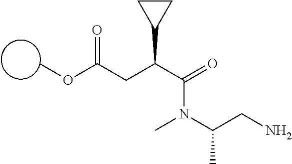 [SMILES: COC(=O)C[C@H](C(=O)N(C)[C@@H](C)CN)C1CC1] | PS-(2-Chlorotrityl) (S)-4-(((S)-1- aminopropan-2- yl)(methyl)amino)-3- cyclopropyl-4- oxobutanoate | AB5 (Example 6.3) |

### Example 6.2: Synthesis of PS-(2-Chlorotrityl) (R)-4-(((S)-1-aminopropan-2-yl)(methyl)amino)-3-benzyl-4-oxobutanoate (AB1)

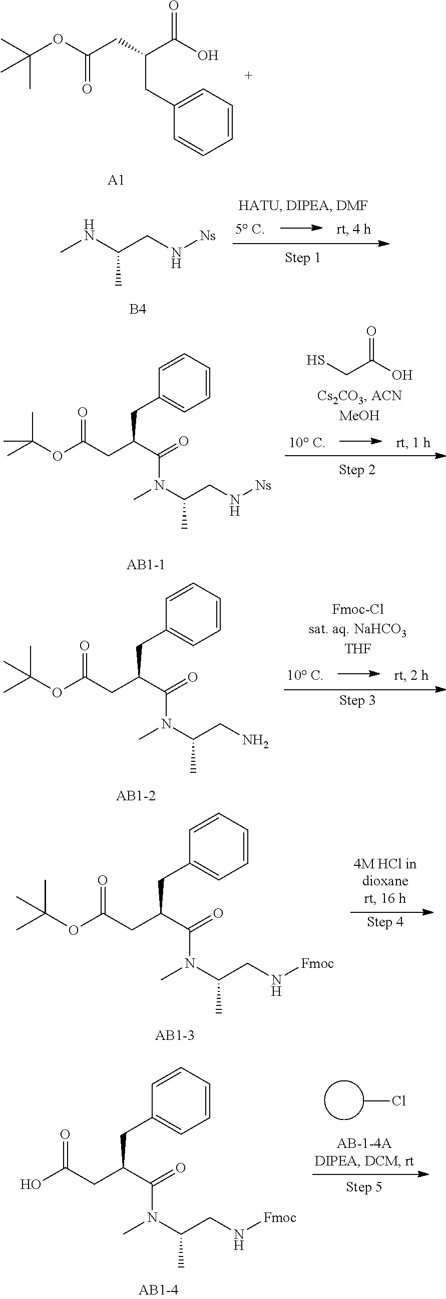

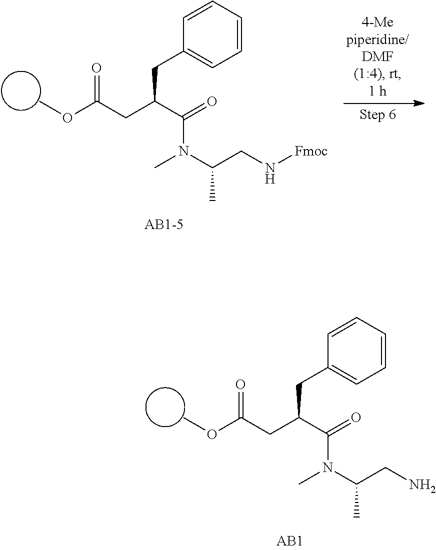

### Step 1. tert-Butyl (R)-3-benzyl-4-(methyl((S)-1-((4-nitrophenyl)sulfonamido)propan-2-yl)amino)-4-oxobutanoate (AB1-1)

To a cold stirred solution of B4 (230 g, 0.742 mol) and A1 (186.4 g, 0.705 mol) in DMF (460 mL) was added DIPEA (388 mL, 2.227 mol) followed by HATU (310.3 g, 0.816 mol) at 5-10° C. The resulting mixture was removed from the cold bath and stirred at rt for 4 h. The reaction mixture was then poured into ice-cold water (5 L) and extracted with ethyl acetate (2×2 L). The combined organic layers were washed with brine solution (2 L), dried over anhydrous Na₂SO₄ and concentrated under vacuum to afford crude AB1-1. The crude product was purified by 230-400-mesh silica gel column chromatography eluting with 20% ethyl acetate in petroleum ether to afford compound AB1-1 (232 g, 60%) as a pale gum solid. Analytical method 7; t_{R}=1.40 min; [M+H]⁺=520.3.

### Step 2. tert-Butyl (R)-4-(((S)-1-aminopropan-2-yl)(methyl)amino)-3-benzyl-4-oxobutanoate (AB1-2)

To a cold stirred solution of AB1-1 (455 g, 0.875 mol) in acetonitrile (3.5 L) and methanol (3.5 L) below 10° C. was added cesium carbonate (1.995 kg, 6.125 mol) and the resulting mixture was stirred for 15 minutes. 2-Mercaptoacetic acid (322.7 g, 3.50 mol) was then added to at the same temperature. The resulting mixture was stirred at room temperature for 1 h and concentrated under reduced pressure to remove the solvent. The crude product was dissolved in water (3 L) and the aqueous phase was extracted with dichloromethane (2×2 L). The combined organic layers were washed with sat. sodium bicarbonate (3 L) and brine solution (2 L), dried over anhydrous Na₂SO₄, filtered, and concentrated under vacuum to afford AB1-2 (300 g, crude) as a yellow oil which used in the next step without purification.

### Step 3. tert-Butyl (R)-4-(((S)-1-((((9H-fluoren-9-yl)methoxy)carbonyl)amino)propan-2-yl)(methyl) amino)-3-benzyl-4-oxobutanoate (AB1-3)

To a cold stirred solution of AB1-2 (270 g, 0.807 mol) in THF (1.5 L) was added Fmoc-Cl (209.1 g, 0.807 mol) portion wise over 30 min at 10° C., followed by sat. sodium bicarbonate solution (3.3 L) over a period of 30 minutes at the same temperature. The mixture was then stirred at room temperature for 2 h. The reaction mixture was diluted with water (2 L) and extracted with ethyl acetate (2×1 L). The combined organic layers were washed with brine solution (2 L), dried over anhydrous Na₂SO₄, filtered, and concentrated under vacuum. The crude material was purified on a 230-400-mesh silica gel column eluting with 20% ethyl acetate in petroleum ether to afford compound AB1-3 (265 g, 59%) as a yellow gum solid. Analytical method 7; t_{R}=1.49 min; [M+H]⁺=557.3.

### Step 4. (R)-4-(((S)-1-((((9H-Fluoren-9-yl)methoxy)carbonyl)amino)propan-2-yl)(methyl)amino)-3-benzyl-4-oxobutanoic acid (AB1-4)

To a solution of AB1-3 (265 g, 0.476 mol) in 1,4-dioxane (530 mL) was added 4 M HCl in dioxane (2.65 L) at room temperature. The resulting mixture was stirred for 16 h and then concentrated under reduced pressure. The crude product was purified on a 230-400-mesh silica gel column eluting with 30% ethyl acetate in petroleum ether to give AB1-4 (200 g, 84%) as an off-white solid. Analytical method 7; t_{R}=1.48 min; [M+H]⁺=501.4.

### Step 5. PS-(2-Chlorotrityl) (R)-4-(((S)-1-((((9H-fluoren-9-yl)methoxy)carbonyl)amino)propan-2-yl)(methyl)amino)-3-benzyl-4-oxobutanoic acid (AB1-5)

2-Chlorotrityl chloride resin (AB-1-4A, 4.27 g, 4.27 mmol) was pre-washed with DCM (3×20 mL). AB1-4 (1.9 g, 3.80 mmol) dissolved in DCM (20 mL) and DIPEA (1.5 mL, 8.59 mmol) were added to the resin. The resulting mixture was shaken at rt for 16 h, then washed with DCM (3×40 mL), and shaken in DCM/MeOH (50 mL/20 mL) for 30 min to cap the resin. The resin was then filtered, washed with DMF (2×50 mL), and DCM (2×50 mL) and dried under vacuum to give AB1-5 resin (6.13 g, crude). The resin was taken onto the next step without purification.

### Step 6. PS-(2-Chlorotrityl) (R)-4-(((S)-1-aminopropan-2-yl)(methyl)amino)-3-benzyl-4-oxobutanoic acid (AB1)

AB1 resin (2.2 g, crude), was prepared according to the procedure in Example 6.1, Step 5 starting from AB1-5 resin (2.85 g, 1.283 mmol). AB1 was taken onto the next step without purification.

### Example 6.3: Synthesis of PS-(2-Chlorotrityl) (R)-4-(((S)-1-amino-3-cyclopropylpropan-2-yl)(methyl)amino)-3-methyl-4-oxobutanoate (AB5)

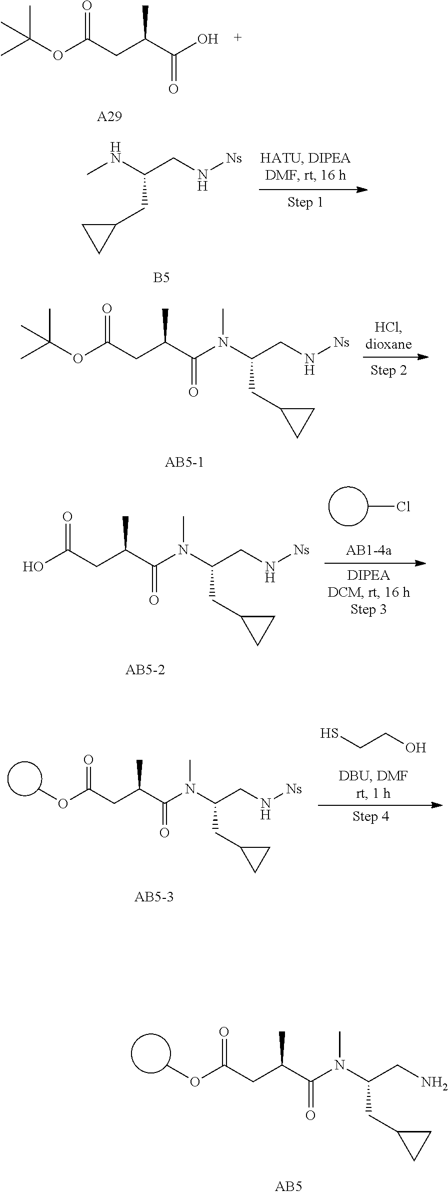

### Step 1. tert-Butyl (R)-4-(((S)-1-cyclopropyl-3-((4-nitrophenyl)sulfonamido)propan-2-yl)(methyl)amino)-3-methyl-4-oxobutanoate (AB5-1)

AB5-1 (700 mg, 48%) was prepared according to the procedure in Example 6.2, Step 1 starting from B5 (1.3 g, 3.72 mmol) and A29 (572 mg, 0.304 mmol). Analytical method 5; t_{R}=1.14 min; [M−H]⁻=482.4.

### Step 2. (R)-4-(((S)-1-Cyclopropyl-3-((4-nitrophenyl)sulfonamido)propan-2-yl)(methyl)amino)-3-methyl-4-oxobutanoic acid (AB5-2)

To a solution of AB5-1 (700 mg, 1.448 mmol) in 1,4-dioxane (5 mL) was added 4 M HCl in dioxane (7.3 mL) at room temperature. The resulting was stirred for 16 h and then concentrated under reduced pressure to afford crude intermediate AB5-2 (619 mg, 100%). Analytical method 5; t_{R}=0.57 min; [M+H]⁺=428.2.

### Step 3. PS-(2-Chlorotrityl) (R)-4-(((S)-1-cyclopropyl-3-((4-nitrophenyl)sulfonamido)propan-2-yl)(methyl)amino)-3-methyl-4-oxobutanoate (AB5-3)

AB5-3 (832 mg, 80%) was prepared according to the procedure in Example 6.2, Step 5 starting from AB5-2 (619 mg, 1.45 mmol). AB5-3 was taken onto the next step without purification.

### Step 4. PS-(2-Chlorotrityl) (R)-4-(((S)-1-amino-3-cyclopropylpropan-2-yl)(methyl)amino)-3-methyl-4-oxobutanoate (AB5)

AB5-3 (832 mg, 1.158 mmol) was suspended in DMF (10 mL) and treated with 2-mercaptoethanol (0.815 mL, 11.58 mmol) and DBU (0.873 mL, 5.79 mmol). The suspension was shaken at room temperature for 1 h. The resin was filtered, washed with NMP (2×10 mL), DMF (2×10 mL), and DCM (3×10 mL) and dried under vacuum to yield AB5 resin (617 mg, crude). The resin was taken onto the next step step without purification.

### Example 6.4: Synthesis of tert-butyl (R)-4-(((1S,2S)-2-((tert-butoxycarbonyl)amino)cyclohexyl)-(methyl)amino)-3-(cyanomethyl)-4-oxobutanoate (AB6-3)

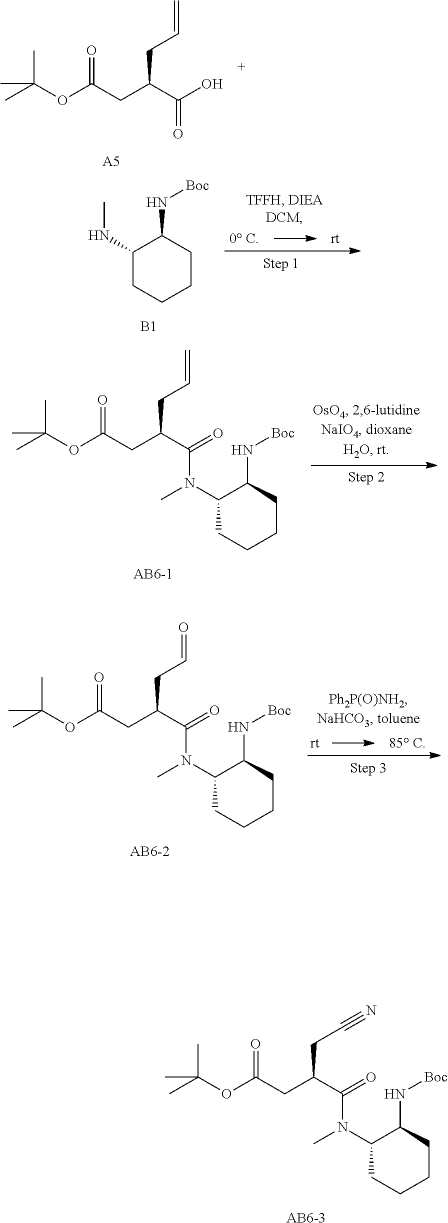

### Step 1. tert-Butyl (R)-3-(((1S,2S)-2-((tert-butoxycarbonyl)amino)cyclohexyl)(methyl)carbamoyl)hex-5-enoate (AB6-1)

To a solution of A5 (286 mg, 1.335 mmol) in DCM (13.3 mL) at 0° C. was added DIEA (0.699 mL, 4.00 mmol), followed by fluoro-N,N,N′,N′-tetramethylformamidinium hexafluorophosphate (441 mg, 1.669 mmol) in three portions over 2 min. The resulting clear pale yellow-orange mixture was stirred for 30 min, and then B1 (442 mg, 1.669 mmol) was added. The resulting mixture was warmed to room temperature slowly and stirred for 48 h. After aqueous workup, the organic residue was purified by normal phase chromatography using a silica gel column, eluting with 0-100% EtOAc in heptanes, to provide AB6-1 (355 mg, 0.836 mmol, 62.6% yield). Analytical method 7; t_{R}=1.24 min, [M+H]⁺=425.3.

### Step 2. tert-Butyl (R)-3-(((1S,2S)-2-((tert-butoxycarbonyl)amino)cyclohexyl)(methyl)carbamoyl)-5-oxopenta-noate (AB6-2)

To a solution of AB6-1 (315 mg, 0.742 mmol) in dioxane:water (3:1, 15 mL) was added 2,6-lutidine (173 μL, 1.484 mmol) followed by a solution of osmium tetroxide (2.5% in tBuOH) (186 μL, 0.015 mmol) and sodium periodate (635 mg, 2.97 mmol), and the resulting mixture was stirred at room temperature for 1 h. Water was added, followed by DCM. The organic layer was separated and the aqueous layer was extracted with DCM (2×). The combined organic layers were washed with 0.1 N HCl (2×), saturated sodium bicarbonate solution and brine, dried over sodium sulfate, filtered, and concentrated to provide AB6-2 (300 mg, 0.703 mmol, 95% yield). The crude material was taken directly to the next step without purification. Analytical method 7; t_{R}=1.10 min, [M+Na]⁺=449.4. Method 5; t_{R}=1.09 min, [M+H]⁺=427.5.

### Step 3. tert-Butyl (R)-4-(((1S,2S)-2-((tert-butoxycarbonyl)amino)cyclohexyl)(methyl) amino)-3-(cyanomethyl)-4-oxobutanoate (AB6-3)

To a solution of AB6-2 (255 mg, 0.598 mmol) in toluene (4 mL) was added (aminooxy)diphenylphosphine oxide (174 mg, 0.747 mmol) followed by sodium bicarbonate (151 mg, 1.793 mmol) and the resulting mixture was stirred at room temperature for 2 h. The reaction mixture was then heated to 85° C. and stirred at that temperature. After 4 h of stirring, an additional equivalent each of (aminooxy)diphenylphosphine oxide and sodium bicarbonate were added, and stirring was continued overnight at 85° C. The reaction mixture was then allowed to cool to room temperature and was diluted with EtOAc and saturated sodium bicarbonate solution. The organic layer was washed with saturated sodium bicarbonate solution and brine, dried over sodium sulfate, filtered, and concentrated. The crude product was purified by normal phase chromatography using a silica column to provide AB6-3 (168 mg, 0.397 mmol, 66% yield). Analytical method 7; t_{R}=1.11 min, [M+Na]⁺=446.2. Method 5; t_{R}=1.12 min, [M+Na]⁺=446.2.

### Example 6.5: Synthesis of 2-((2S,5S)-5-(((((9H-fluoren-9-yl)methoxy)carbonyl)amino)methyl)-1,4-dimethyl-3,6-dioxopiperazin-2-yl)acetic acid (AB7-4)

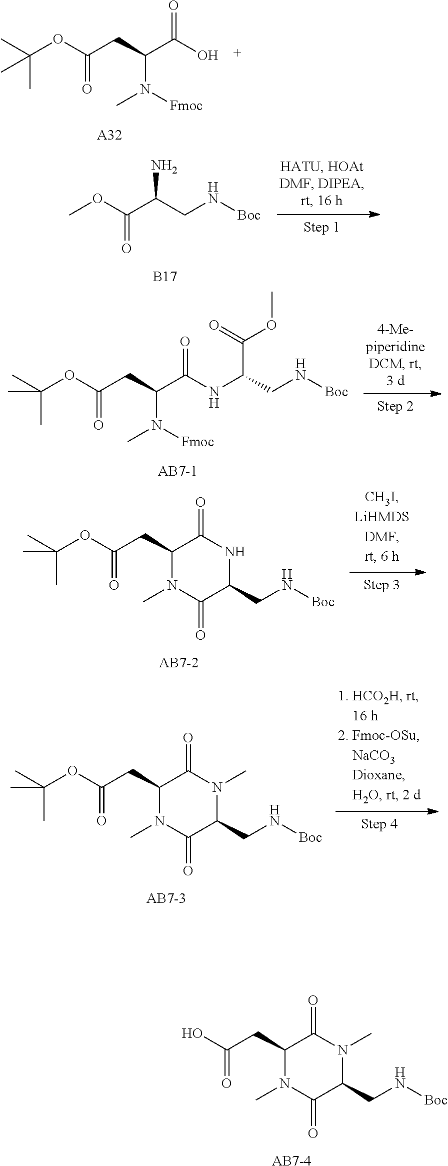

### Step 1. (S)-tert-Butyl 3-((((9H-fluoren-9-yl)methoxy)carbonyl)(methyl)amino)-4-(((S)-3-((tert-butoxy-carbonyl)amino)-1-methoxy-1-oxopropan-2-yl)amino)-4-oxobutanoate (AB7-1)

To A32 (0.97 g, 2.29 mmol) dissolved in DMF (11 mL) and HOAt (0.03 g, 0.23 mmol) was added HATU (0.87 g, 2.29 mmol) and DIEA (2 mL) and the resulting solution was stirred for 5 min. B17 (0.58 g, 2.29 mmol) was then added. The reaction mixture was stirred for 16 h and then partitioned between 0.1 M HCl and EtOAc. The organic layer was washed with 10% LiCl (3×), dried over Na₂SO₄, filtered, and concentrated under vacuum to provide Ab7-1 as a white solid which was taken onto the next step without purification. Analytical method 1; t_{R}=1.75 min, [M+H]⁺=626.0.

### Step 2. tert-Butyl 2-((2S,5S)-5-(((tert-butoxycarbonyl)amino)methyl)-1-methyl-3,6-dioxopiperazin-2-yl)acetate (AB7-2)

AB7-1 (1.4 g, 2.29 mmol) was suspended in DCM (18 mL), and 4-methylpiperidine (4.5 mL) was slowly added. The solution cleared, turned yellow and was stirred at rt for 3 days. The solution was concentrated under vacuum and purified by column chromatography (SiO₂, Heptane/EtOAc, eluting with 80%-100% EtOAc). Material with Rf of 0.25 in 100% EtOAc was collected (TLC stain: phosphomolybdic acid) to obtain 201 mg (24%) of the desired cis-isomer AB7-2 as a colorless solid. ¹H NMR (400 MHz, DMSO-d₆) δ 8.20-8.12 (m, 1H), 6.84 (d, J=6.1 Hz, 1H), 4.23-4.14 (m, 1H), 3.88 (s, 1H), 3.31 (m, 2H), 3.28-3.18 (m, 1H), 1.99 (s, 3H), 1.41 (s, 9H), 1.38 (s, 9H).

### Step 3. tert-Butyl 2-((2S,5S)-5-(((tert-butoxycarbonyl)amino)methyl)-1,4-dimethyl-3,6-dioxopiperazin-2-yl)acetate (AB7-3)

To lactam AB7-2 (170 mg, 0.46 mmol) dissolved in DMF (4.2 mL) and THF (0.4 mL) was added Mel (65 mg, 0.46 mmol), followed by LiHMDS (77 mg, 0.458 mmol). The resulting mixture was stirred at rt for 6 h and then quenched with water and EtOAc. The organic layer was washed with 10% LiCl (2×), dried over Na₂SO₄, filtered, and concentrated under reduced pressure. The crude product was purified via silica gel chromatography (eluting with Heptane/EtOAc, 90-100% EtOAc) to provide AB7-3 as a yellow oil (155 mg, 88%).

### Step 4. 2-((2S,5S)-5-(((((9H-Fluoren-9-yl)methoxy)carbonyl)amino)methyl)-1,4-dimethyl-3,6-dioxopiperazin-2-yl)acetic acid (AB7-4)

AB7-3 (140 mg, 0.36 mmol) was stirred in 5.5 mL of formic acid at rt for 16 h. The reaction mixture was concentrated under vacuum and then taken up in water and lyophilized. The material obtained was suspended in 7.2 mL dioxane and Na₂CO₃ (1 M, 1.8 mL) was added. The resulting solution was cooled to 0° C. and Fmoc-OSu (121 mg, 0.36 mmol, in 1.8 mL dioxane) was added. After stirring the solution for 2 days, 1 M HCl (5 mL) was added, and the organic volatiles were removed under vacuum. The obtained residue was purified by column chromatography on SiO₂ (eluting with DCM/MeOH (0-30% MeOH)) to provide AB7-4 as a colorless solid (84 mg, 52%). Analytical method 1; t_{R}=1.14 min, [M+H]⁺=452.0. ¹H NMR (400 MHz, DMSO-d₆) δ 7.88 (d, J=7.5 Hz, 2H), 7.78-7.60 (m, 3H), 7.41 (t, J=7.4 Hz, 2H), 7.32 (t, J=6.9 Hz, 2H), 4.37-4.15 (m, 4H), 4.01 (s, 1H), 3.66-3.53 (m, 1H), 3.43-3.34 (m, 1H), 2.85 (s, 2H), 2.82 (s, 3H).

### Example 6.6: Synthesis of tert-butyl (R)-4-((1-(((((9H-fluoren-9-yl)methoxy)carbonyl)amino)-methyl)cyclopropyl)-(methyl)amino)-3-benzyl-4-oxobutanoate (AB4-1)

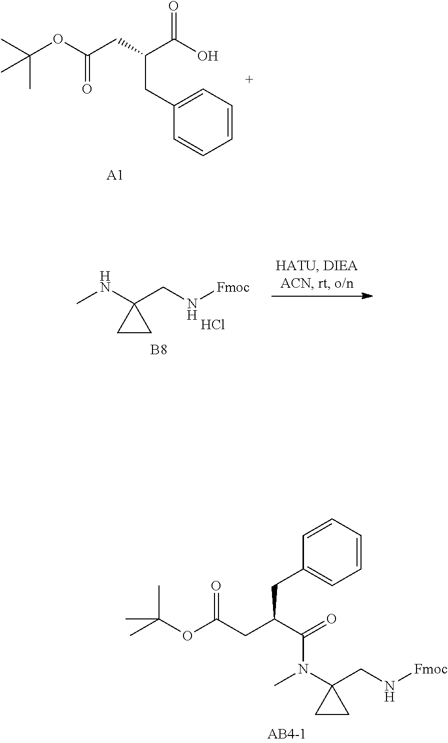

To a flask containing B8 (794 mg, 2.21 mmol), A1 (585 mg, 2.21 mmol), HATU (1.01 g, 2.66 mmol) and HOAt (512 mg, 3.76 mmol) was added ACN (15 mL), followed by DIPEA (1.24 mL, 7.08 mmol) and the resulting mixture was stirred at rt for overnight. The reaction mixture was the concentrated to dryness and the crude material purified by flash column on silica gel (eluting with EtOAc/Heptane, 0-30%) to afford AB4-1 after concentrating the pure fractions under reduced pressure (863 mg, 65% yield). Analytical method 1; t_{R}=1.87 min; [M+H]⁺=569.3.

### Example 6.7: Synthesis of (3S,4R)-4-((tert-butyldimethylsilyl)oxy)-3-(methyl((S)-1-((4-nitrophenyl)sulfonamido)-propan-2-yl)carbamoyl)heptanoic acid (AB8-6)

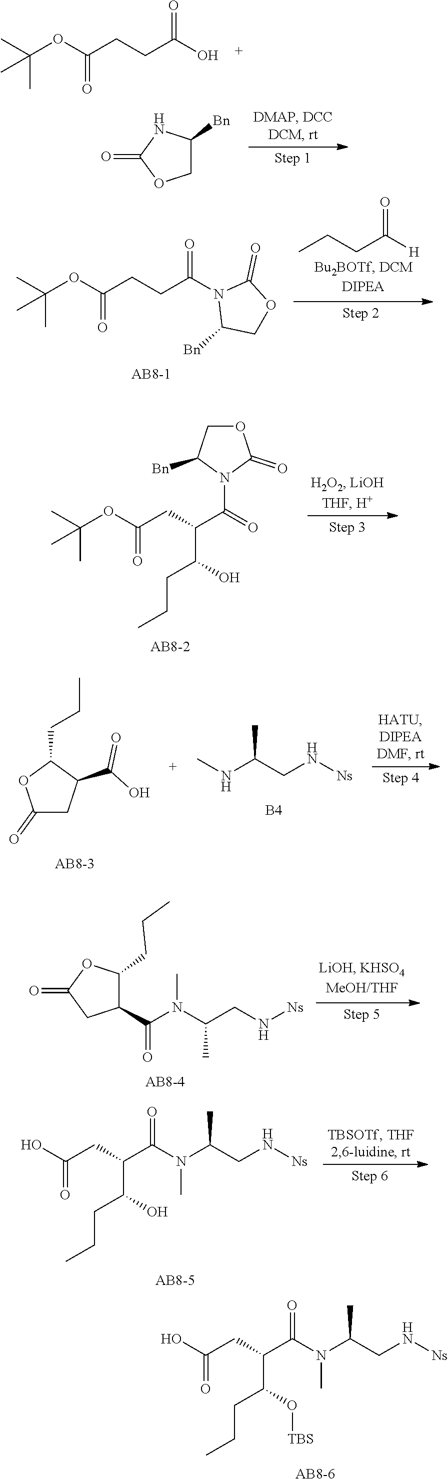

### Step 1. (S)-tert-Butyl 4-(4-benzyl-2-oxooxazolidin-3-yl)-4-oxobutanoate (AB8-1)

AB8-1 (1.2 g, 63%) was prepared according to the procedure for Example 4.4, Step 1 starting from 4-(tert-butoxy)-4-oxobutanoic acid (1 g, 5.74 mmol) and (S)-4-benzyloxazolidin-2-one (1.32 g, 7.46 mmol). Analytical method 5; t_{R}=1.08 min; MS [M+Na]⁺=356.1.

### Step 2. (3S,4R)-tert-Butyl 3-((S)-4-benzyl-2-oxooxazolidine-3-carbonyl)-4-hydroxyheptanoate (AB8-2)

To a flask containing AB8-1 (500 mg, 1.50 mmol) and DCM (8 mL) cooled to −78° C. was added solution of dibutylboryl trifluoromethanesulfonate (1 M in DCM, 1.65 mL, 1.65 mmol) dropwise and the resulting mixture was stirred at −78° C. for 30 min. DIPEA (0.31 mL, 1.80 mmol) was then added dropwise and stirred for another 45 min. A solution of butyraldehyde (151 mg, 2.10 mmol) in 1.5 mL DCM was added dropwise and stirred at −78° C. for 3 h and then warmed to −15° C. The reaction was quenched with water and then stirred at rt to afford a biphasic layer. The organic phase was concentrated and purified by by normal phase column chromatography using a silica gel column (eluting with DCM/EtOAc) to afford AB8-2 (319 mg, 53% yield). Analytical method 5; t_{R}=1.14 min; [M+Na]⁺=428.3.

### Step 3. (2R,3S)-5-Oxo-2-propyltetrahydrofuran-3-carboxylic acid (AB8-3)

To a flask containing AB8-2 (219 mg, 0.54 mmol) in THF (6 mL) cooled in an ice bath was added a solution of H₂O₂(35% aq, 0.19 mL, 2.16 mmol) and LiOH (0.5 M, 2.2 mL, 1.08 mmol) and the resulting mixture was stirred in an ice bath for 1.5 h. A solution of sodium sulfite (0.8 M, 4.05 mL, 3.24 mmol) was then added and the reaction mixture was warmed to rt, stirred for 30 min, and then concentrated to remove excess THF to afford a mostly aqueous mixture. A small amount of saturated sodium bicarbonate solution was added and the mixture was washed twice with DCM. The resulting aqueous portion was collected and the pH was adjusted with 4 N HCl to pH ˜2. The mixture was extracted with EtOAc twice, and the combined organic phases were washed with brine, dried over sodium sulfate, filtered, and concentrated to afford AB8-3 as solid (76 mg, 82% yield).

### Step 4. (2R,3S)—N-Methyl-N—((S)-1-(4-nitrophenylsulfonamido)propan-2-yl)-5-oxo-2-propyltetrahydrofuran-3-carboxamide (AB8-4)

AB8-4 (117 mg, 62%) was prepared according to the procedure used in Example 6.5, Step 1 starting from AB8-3 (76 mg, 0.44 mmol) and B4 (121 mg, 0.44 mmol). Analytical method 5; t_{R}=0.90 min; [M+H]⁺=428.2.

### Step 5. (3S,4R)-4-Hydroxy-3-(methyl((S)-1-(4-nitrophenylsulfonamido)propan-2-yl)carbamoyl)heptanoic acid (AB8-5)

To a flask containing AB8-4 (117 mg, 0.27 mmol) in THF (1.5 mL) and MeOH (0.5 mL) at rt was added a solution of LiOH (0.93 mL, 0.47 mmol) the resulting mixture was heated to 50° C. overnight. The reaction mixture was cooled to rt and concentrated. The crude material was taken up in water and the pH was adjusted with a 1 N solution of KHSO₄ to pH of 3 to 4. The mixture was then freeze-dried to afford AB8-5 as a crude powder. The material was carried onto the next step without purification (122 mg, assume quantitative yield). Analytical method 7; t_{R}=0.77 min; [M+H]⁺=446.2.

### Step 6. (3S,4R)-4-((tert-Butyldimethylsilyl)oxy)-3-(methyl((S)-1-(4-nitrophenylsulfonamido)propan-2-yl)-carbamoyl)heptanoic acid (AB8-6)

To a flask containing AB8-5 (122 mg, 0.27 mmol) in THF (3 mL) cooled in an ice bath was added 2,6-lutidine (0.16 mL, 1.37 mmol), followed by TBS-OTf (0.22 mL, 0.96 mmol) and the resulting mixture was warmed to rt and stirred for 1.5 h. MeOH was then added and the reaction mixture was stirred for 30 min and then concentrated under reduced pressure. The obtained residue was then taken up in ACN and treated with an aq. solution of 10% citric acid in water for 1 hour. EtOAc was then added and the aqueous phase was extracted with more EtOAc. The organic phase was dried over sodium sulfate, filtered, and concentrated to afford the crude product. The material was combined with a previous batch and purified by reverse phase flash C18 column (eluting with water/ACN, neutral) to afford AB8-6 (166 mg, 81% yield). Analytical method 7; t_{R}=1.25 min; [M+H]⁺=560.1.

### Example 7: Building Block AB

### Example 7.1: Synthesis of tert-butyl (R)-3-((pyridinoxide-2-yl)methyl)-4-(((1S,2S)-2-aminocyclohexyl)(methyl)-amino)-4-oxobutanoate (AB50)

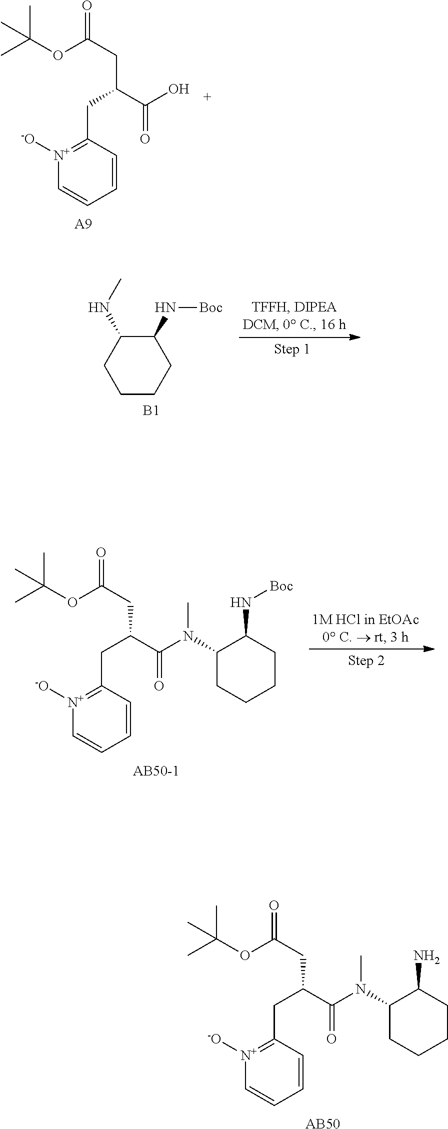

### Step 1. tert-Butyl (R)-3-((pyridinoxide-2-yl)methyl)-4-(((1S,2S)-2-((tert-butoxycarbonyl)amino)-cyclohexyl)-(methyl)amino)-4-oxobutanoate (AB50-1)

To a solution of Intermediate A9 (560 mg, 1.79 mmol) in DCM (200 mL) was added DIPEA (0.94 mL, 5.37 mmol) and TFFH (709 mg, 2.685 mmol) at 0° C. and the resulting mixture was stirred for 30 min. Intermediate B1 (474 mg, 1.79 mmol) was added. The resulting mixture was warmed to rt slowly and then stirred at room temperature overnight. The reaction mixture was concentrated down to 20 mL of DCM and then diluted with EtOAc (200 mL). The organic layer was washed with sat. aq. NaHCO₃ (3×200 mL), water, and brine, dried (Na₂SO₄), filtered, and concentrated in vacuo. The crude product was purified via normal phase chromatography using an ISCO 40 g Silica gel column eluting with 0-50% EtOAc/heptane to afford AB50-1 (778 mg, 88%) as a white solid. Analytical method 2; t_{R}=2.15 min; [M+H]⁺=492.2.

### Step 2. 2-((R)-2-(((1S,2S)-2-aminocyclohexyl)(methyl)carbamoyl)-4-(tert-butoxy)-4-oxobutyl)pyridine 1-oxide (AB50)

To a solution of AB50-1 (778 mg, 1.583 mmol) in EtOAc (1 mL) was added 1 M HCl in EtOAc (15.83 mL, 15.83 mmol) at 0° C. The reaction mixture was then stirred at rt for 3 h and then concentrated under reduced pressure at rt. The obtained residue was diluted with EtOAc. The organic layer was dried over Na₂SO₄, filtered, and concentrated under reduced pressure to give AB50 (281 mg, 45.4%). Analytical method 7; t_{R}=0.71 min; [M+H]⁺=392.7.

The Building Blocks AB (BB AB) in Table 21 below were used for the assembly of the linear pentamer compounds by solution phase chemistry. They were prepared according to the procedure used for AB50 (Example 7.1), AB55 (Example 7.2), AB65 & AB66 (Example 7.3), AB70 (Example 7.4), AB72 (Example 7.5), or AB70-2 (Example 7.4, Step 1-2), from the monomer building blocks listed in Table 17 and Table 19.

TABLE 21
Building block AB - Polymer-bound dimers

| | | | Synthesis |
|---|---|---|---|
| BB | | | procedure/ |
| No. | Structure | Compound Name | LCMS |
| AB51 | 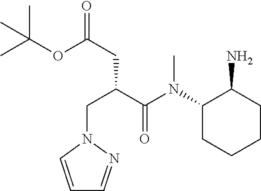 [SMILES: CN(C(=O)[C@@H](CC(=O)OC(C)(C)C)Cn1cccn1)[C@H]1CCCC[C@@H]1N] | tert-butyl (S)-3-((1H-pyrazol- 1-yl)methyl)-4-(((1S,2S)-2- aminocyclohexyl)(methyl) amino)-4-oxobutanoate | AB50 (Example 7.1)-starting from A4 and B1/ Analytical method 5 t_{R} = 0.88 min [M + H]⁺ = 365.3 |
| AB52 | 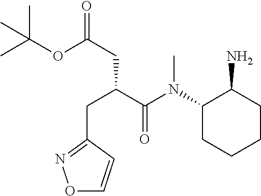 [SMILES: CN(C(=O)[C@@H](CC(=O)OC(C)(C)C)Cc1ccon1)[C@H]1CCCC[C@@H]1N] | tert-butyl (R)-4-(((1S,2S)-2- aminocyclohexyl)(methyl) amino)-3-(isoxazol-3-yl- methyl)-4-oxobutanoate | AB50 (Example 7.1)-starting from A15 and B1/ Analytical method 5 t_{R} = 0.88 min [M + H]⁺ = 366.0 |
| AB53 | 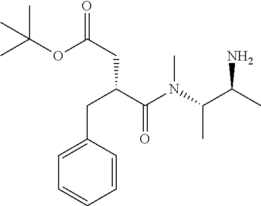 [SMILES: C[C@H](N)[C@H](C)N(C)C(=O)[C@@H](CC(=O)OC(C)(C)C)Cc1ccccc1] | tert-butyl (R)-4-(((2S,3S)-3- aminobutan-2- yl)(methyl)amino)-3-benzyl- 4-oxobutanoate | AB50 (Example 7.1)-starting from A1 and B9/ Analytical method 7 t_{R} = 1.27min [M-tBu]⁺ = 293.0 |
| AB54 | 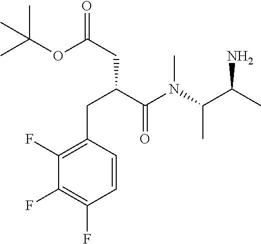 [SMILES: C[C@H](N)[C@H](C)N(C)C(=O)[C@@H](CC(=O)OC(C)(C)C)Cc1ccc(F)c(F)c1F] | tert-butyl (R)-4-(((2S,3S)-3- aminobutan-2- yl)(methyl)amino)-4-oxo-3- (2,3,4- trifluorobenzyl)butanoate | AB50 (Example 7.1)-starting from A13 and B9/ Analytical method 5 t_{R} = 1.05 min [M + H]⁺ = 403.6 |
| AB56 | 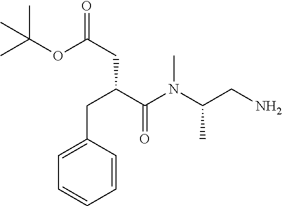 [SMILES: C[C@@H](CN)N(C)C(=O)[C@@H](CC(=O)OC(C)(C)C)Cc1ccccc1] | tert-butyl (R)-4-(((S)-1- aminopropan-2- yl)(methyl)amino)-3-benzyl- 4-oxobutanoate | AB55 (Example 7.2)-starting from A1 and B4/ Analytical method 5 t_{R} = 0.91 min [M + H]⁺ = 335.2 |
| AB57 | 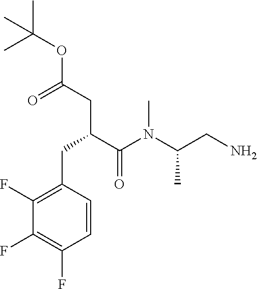 [SMILES: C[C@@H](CN)N(C)C(=O)[C@@H](CC(=O)OC(C)(C)C)Cc1ccc(F)c(F)c1F] | tert-butyl (R)-4-(((S)-1- aminopropan-2- yl)(methyl)amino)-4-oxo-3- (2,3,4- trifluorobenzyl)butanoate | AB55 (Example 7.2)-starting from A13 and B4/ Analytical method 5 t_{R} = 1.01 min [M + H]⁺ = 389.3 |
| AB58 | 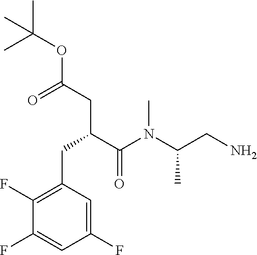 [SMILES: C[C@@H](CN)N(C)C(=O)[C@@H](CC(=O)OC(C)(C)C)Cc1cc(F)cc(F)c1F] | tert-butyl (R)-4-(((S)-1- aminopropan-2- yl)(methyl)amino)-4-oxo-3- (2,3,5- trifluorobenzyl)butanoate | AB55 (Example 7.2)-starting from A12 and B4/ Analytical method 5 t_{R} = 1.02 min [M + H]⁺ = 389.3 |
| AB59 | 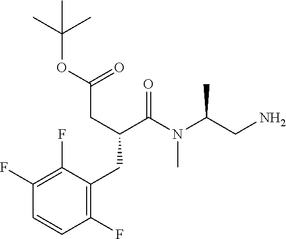 [SMILES: C[C@@H](CN)N(C)C(=O)[C@@H](CC(=O)OC(C)(C)C)Cc1c(F)ccc(F)c1F] | tert-butyl (R)-4-(((S)-1- aminopropan-2- yl)(methyl)amino)-4-oxo-3- (2,3,6- trifluorobenzyl)butanoate | AB55 (Example 7.2)-starting from A11 and B4/ Analytical method 5 t_{R} = 0.96 min [M + H]⁺ = 389.1 |
| AB60 | 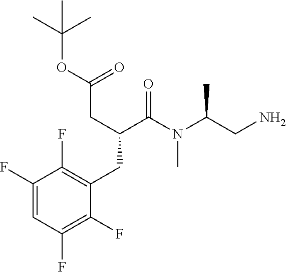 [SMILES: C[C@@H](CN)N(C)C(=O)[C@@H](CC(=O)OC(C)(C)C)Cc1c(F)c(F)cc(F)c1F] | tert-butyl (R)-4-(((S)-1- aminopropan-2- yl)(methyl)amino)-4-oxo-3- (2,3,5,6- tetrafluorobenzyl)butanoate | AB55 (Example 7.2)-starting from A14 and B4/ Analytical method 5 t_{R} = 0.98 min [M + H]⁺ = 407.1 |
| AB61 | 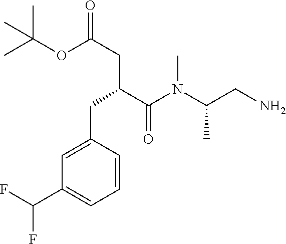 [SMILES: C[C@@H](CN)N(C)C(=O)[C@@H](CC(=O)OC(C)(C)C)Cc1cccc(C(F)F)c1] | tert-butyl (R)-4-(((S)-1- aminopropan-2- yl)(methyl)amino)-3-(3- (difluoromethyl)benzyl)-4- oxobutanoate | AB55 (Example 7.2)-starting from A17 and B4/ Analytical method 5 t_{R} = 0.95 min [M + H]⁺ = 385.2 |
| AB62 | 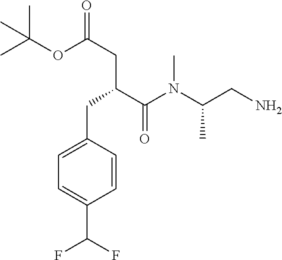 [SMILES: C[C@@H](CN)N(C)C(=O)[C@@H](CC(=O)OC(C)(C)C)Cc1ccc(C(F)F)cc1] | tert-butyl (R)-4-(((S)-1- aminopropan-2- yl)(methyl)amino)-3-(4- (difluoromethyl)benzyl)-4- oxobutanoate | AB55 (Example 7.2)-starting from A18 and B4/ Analytical method 5 t_{R} = 0.95 min [M + H]⁺ = 385.3 |
| AB63 | 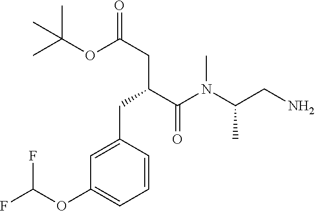 [SMILES: C[C@@H](CN)N(C)C(=O)[C@@H](CC(=O)OC(C)(C)C)Cc1cccc(OC(F)F)c1] | tert-butyl (R)-4-(((S)-1- aminopropan-2- yl)(methyl)amino)-3-(3- (difluoromethoxy)benzyl)-4- oxobutanoate | AB55 (Example 7.2)-starting from A22 and B4/ Analytical method 5 t_{R} = 1.01 min [M + H]⁺ = 401.3 |
| AB64 | 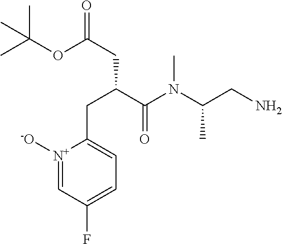 | 2-((R)-2-(((S)-1- aminopropan-2- yl)(methyl)carbamoyl)-4- (tert-butoxy)-4-oxobutyl)-5- fluoropyridine 1-oxide | AB55 (Example 7.2)-starting from A8 and B4/ Analytical method 7 t_{R} = 0.59 min [M + H]⁺ = 370.3 |
| AB67 | 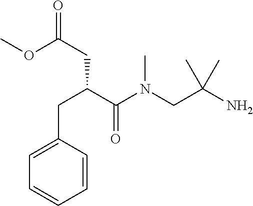 [SMILES: COC(=O)C[C@@H](Cc1ccccc1)C(=O)N(C)CC(C)(C)N] | methyl (R)-4-((2-amino-2- methylpropyl)(methyl)amino)- 3-benzyl-4-oxobutanoate | AB65 & AB66 (Example 7.3)- starting from A1 and B7/Analytical method 5 t_{R} = 0.79 min [M + H]⁺ = 306.8 |
| AB68 | 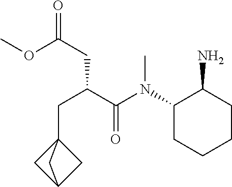 | methyl (R)-4-(((1S,2S)-2- aminocyclohexyl)(methyl) amino)-3-(bicyclo[1.1.1] pentan-1-ylmethyl)-4-oxo- butanoate | AB65 & AB66 (Example 7.3)- starting from A27 and B1/ Analytical method 5 t_{R} = 1.35 min [M + H]⁺ = 465.4 |
| AB69 | 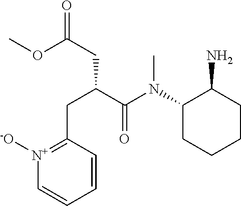 | 2-((R)-2-(((1S,2S)-2- aminocyclohexyl)(methyl) carbamoyl)-4-methoxy-4- oxobutyl)pyridine 1-oxide | AB65 & AB66 (Example 7.3)- starting from A9 and B1/Analytical method 5 t_{R} = 0.54 min [M + H]⁺ = 349.9 |
| AB71 | 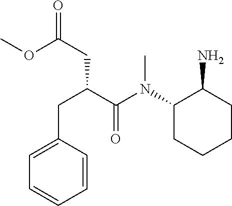 [SMILES: COC(=O)C[C@@H](Cc1ccccc1)C(=O)N(C)[C@H]1CCCC[C@@H]1N] | methyl (R)-4-(((1S,2S)-2- aminocyclohexyl)(methyl) amino)-3-benzyl-4-oxo- butanoate | AB70 (Example 7.4)-starting from A1 and B1/ Analytical method 5 t_{R} = 0.85 min [M + H]⁺ = 333.5 |
| AB73 | 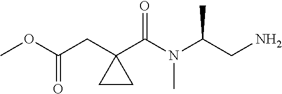 [SMILES: COC(=O)CC1(C(=O)N(C)[C@@H](C)CN)CC1] | methyl (S)-2-(1-((1- aminopropan-2- yl)(methyl)carbamoyl) cyclopropyl) acetate | AB72 (Example 7.5)-starting from A29 and B15/ Analytical method 5 t_{R} = 0.47 min [M + H]⁺ = 229.0 |
| AB75 | 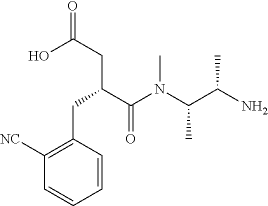 [SMILES: C[C@H](N)[C@H](C)N(C)C(=O)[C@@H](CC(=O)O)Cc1ccccc1C#N] | (R)-4-(((2S,3S)-3- aminobutan-2- yl)(methyl)amino)-3-(2- cyanobenzyl)-4-oxobutanoic acid | AB70-2 (Example 7.4, Step 1-2)- starting from A31 and B9/Analytical method 5 t_{R} = 0.44 min [M + H]⁺ = 317.8 |

### Example 7.2: Synthesis of tert-butyl (R)-3-(((S)-1-aminopropan-2-yl)(methyl)carbamoyl)hex-5-enoate (AB55)

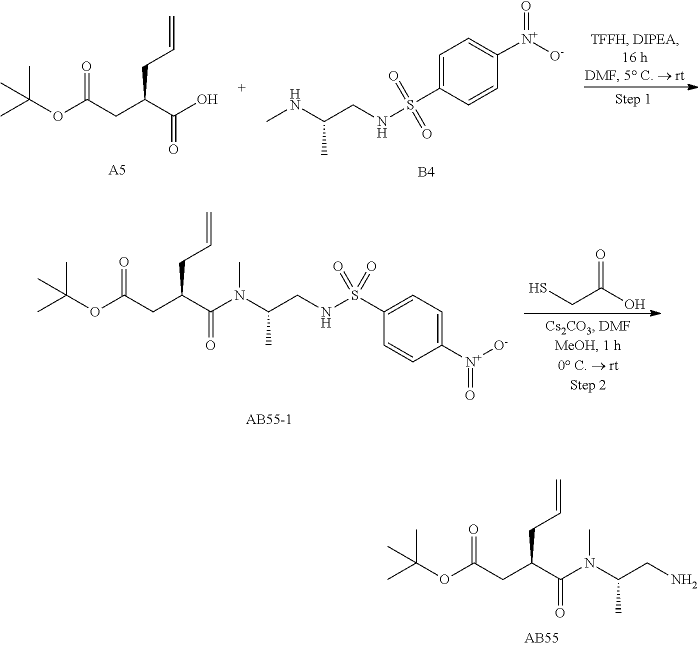

### Step 1. tert-Butyl (R)-3-(methyl((S)-1-((4-nitrophenyl)sulfonamido)propan-2-yl)carbamoyl)hex-5-enoate (AB55-1)

To a solution of (R)-2-(2-(tert-butoxy)-2-oxoethyl)pent-4-enoic acid A5 (332 mg, 1.550 mmol) in DCM (15 mL) was added DIPEA (0.541 mL, 3.10 mmol) and TFFH (409 mg, 1.550 mmol) at 0° C. The resulting mixture was stirred for 30 min until dissolved and then intermediate B4 (320 mg, 1.033 mmol) in DCM (15 mL) was added. The ice bath was removed and the reaction mixture was stirred at room temperature overnight, then concentrated to 5 mL DCM and diluted with EtOAc (25 mL). The organic layer was washed with sat. NaHCO₃ (25 mL), water (25 mL), and brine (25 mL), dried over Na₂SO₄, filtered, and concentrated under reduced pressure which provided a yellow oil. The crude material was purified via normal phase chromatography using a 80 g Silica gel column eluting with 0-50% EtOAc in heptane to yield intermediate AB55-1 (200 mg, 39%) as a clear oil. Analytical method 2; t_{R}=2.44 min; [M−H]⁻=468.4.

### Step 2. tert-butyl (R)-3-(((S)-1-aminopropan-2-yl)(methyl)carbamoyl)hex-5-enoate (AB55)

To a cold stirred solution of AB55-1 (200 mg, 0.426 mmol) in DMF (3 mL) and methanol (4 mL) was added cesium carbonate (971 mg, 2.98 mmol) at <10° C. and the resulting mixture was stirred for 15 minutes. 2-Mercaptoacetic acid (0.182 mL, 2.56 mmol) was added at the same temperature and the resulting mixture was stirred at room temperature for 1 h and then concentrated under reduced pressure. The obtained residue was dissolved in water (30 mL) and the aqueous phase was extracted with dichloromethane (2×20 mL). The combined organic layers were washed with sat. aq. sodium bicarbonate (30 mL) and brine solution (20 mL), dried over anhydrous Na₂SO₄, filtered, and concentrated under vacuum to afford crude AB55 (121 mg, 100%) as a yellow oil. Analytical method 4; t_{R}=0.94 min; [M+H]⁺=285.3. The crude material was carried onto the next step without purification.

### Example 7.3: Synthesis of methyl (R)-4-(((3R,4S)-3-aminotetrahydro-2H-pyran-4-yl)(methyl)amino)-3-benzyl-4-oxobutanoate (AB65) and methyl (R)-4-(((3S,4R)-3-aminotetrahydro-2H-pyran-4-yl)(methyl)amino)-3-benzyl-4-oxobutanoate (AB65 and AB66)

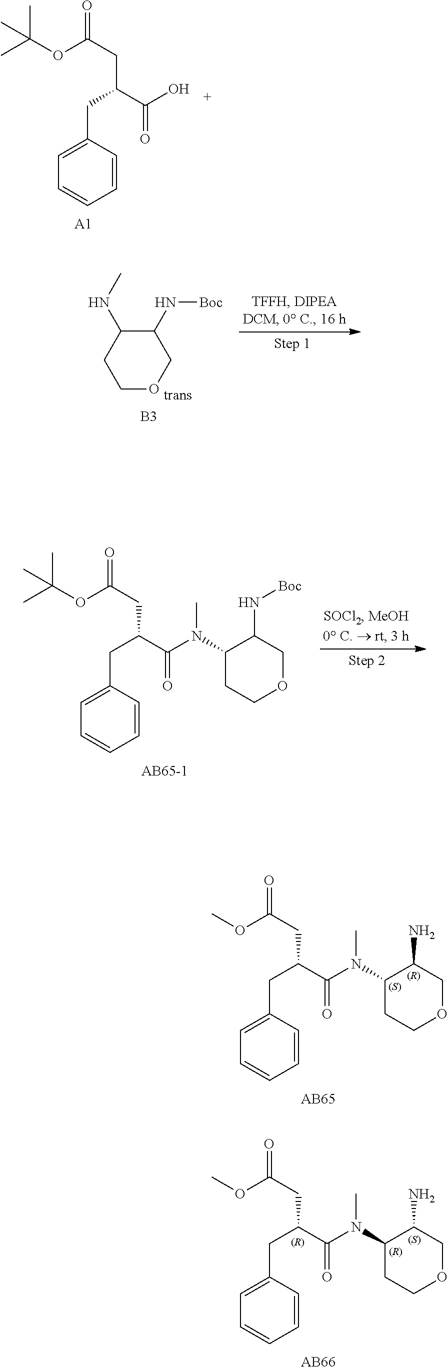

### Step 1. tert-Butyl (3R)-3-benzyl-4-((3-((tert-butoxycarbonyl)amino)tetrahydro-2H-pyran-4-yl)(methyl)amino)-4-oxobutanoate (AB65-1)

To a solution of A1 (252 mg, 0.955 mmol) in DCM (15 mL) was added DIPEA (0.501 mL, 2.87 mmol) and TFFH (124 mg, 1.051 mmol) at 0° C. The resulting mixture was stirred for 30 min and then B3 (220 mg, 0.955 mmol) was added. The resulting mixture was warmed to rt slowly, stirred at room temperature overnight, then concentrated down to 2 mL DCM and diluted with EtOAc (20 mL). The organic layer was washed with sat. aq. NaHCO₃ (3×20 mL), water, and brine then dried (Na₂SO₄), filtered, and concentrated in vacuo. The crude oil was purified via normal phase chromatography using an ISCO 40 g silica gel column eluting with 0-50% EtOAc/heptane to afford AB65-1 (330 mg, 73%) as a white solid. Analytical method 7; t_{R}=1.17 min; [M+H]⁺=477.5.

### Step 2. Methyl (R)-4-(((3R,4S)-3-aminotetrahydro-2H-pyran-4-yl)(methyl)amino)-3-benzyl-4-oxobutanoate (AB65) and methyl (R)-4-(((3S,4R)-3-aminotetrahydro-2H-pyran-4-yl)(methyl)amino)-3-benzyl-4-oxobutanoate (AB65 and AB66)

To a solution of AB65-1 (320 mg, 0.671 mmol) in anhydrous MeOH (4 mL) was added thionyl chloride (0.2 ml, 2.8 mmol) at 0° C. The reaction mixture was stirred at rt for 16 h and then concentrated under reduced pressure at rt. Toluene was added and the resulting mixture was concentrated at rt. The obtained residue was dried to give a diastereomeric mixture of AB65 and AB66 (250 mg). Analytical method 5; showed two peaks t_{R}=0.88 min and t_{R}=0.91 min, [M+H]⁺=335.2. The mixture was separated by SFC (superfluid chromatography; Column: Lux Cellulose-4 30×250 mm) eluted by CO₂ (80 g/min) with 5-55% methanol as cosolvent to afford AB65 (90 mg, LCMS method 5, t_{R}=0.69 min, [M+H]⁺=335.2) and AB66 (90 mg, LCMS method 5, t_{R}=0.73 min, [M+H]⁺=335.1).

### Example 7.4: Synthesis of methyl (R)-4-(((1S,2S)-2-aminocyclohexyl)(methyl)amino)-3-((6-methylpyridin-2-yl)methyl)-4-oxobutanoate (AB70)

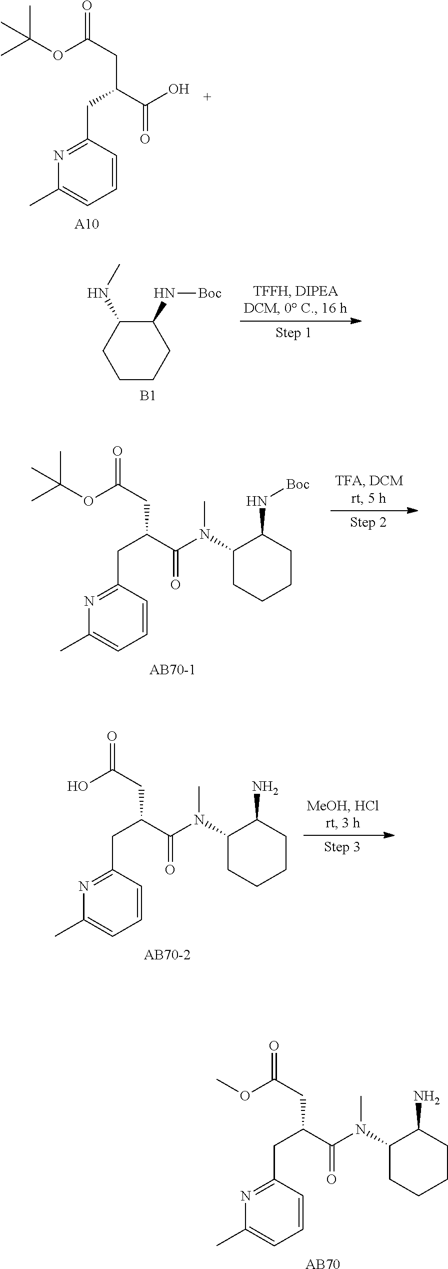

### Step 1. tert-Butyl tert-butyl (R)-4-(((1S,2S)-2-((tert-butoxycarbonyl)amino)cyclohexyl)(methyl)amino)-3-((6-methylpyridin-2-yl)methyl)-4-oxobutanoate (AB70-1)

AB70-1 (197 mg, 20.4%) was prepared according to the procedure in Example 7.3, Step 1 with starting from B1 (450 mg, 1.97 mmol) and A10 (550 mg, 1.97 mmol). Analytical method 5; t_{R}=1.20 min; [M+H]⁺=490.4.

### Step 2. (R)-4-(((1S,2S)-2-Aminocyclohexyl)(methyl)amino)-3-((6-methylpyridin-2-yl)methyl)-4-oxobutanoic acid trifluoroacetate (AB70-2)

AB70-1 (197 mg, 0.402 mmol) in 20% TFA in DCM (10 mL) at rt was stirred for 5 h and the reaction mixture was then concentrated in vacuo. The crude oil was taken up in DCM (10 mL) and concentrated again. This was repeated with fresh DCM (2×10 mL) to yield AB70-2 (TFA salt, 226 mg, 100%) as a white solid. Analytical method 5; t_{R}=0.54 min; [M+H]⁺=334.2.

### Step 3. Methyl (R)-4-(((1S,2S)-2-aminocyclohexyl)(methyl)amino)-3-((6-methylpyridin-2-yl)methyl)-4-oxobutanoate (AB70)

To a solution of AB70-2 (225 mg, 0.4 mmol) in anhydrous MeOH (8 mL) was added 4 M HCl in dioxane (2 mL, 8.00 mmol). The resulting mixture was stirred at rt for 3 h and then concentrated under reduced pressure. The obtained residue was partitioned between EtOAc and NaHCO₃ (aq) and the aqueous layer was extracted with EtOAc. The combined organic layers were dried over Na₂SO₄, filtered, and concentrated under reduced pressure to give AB70 (139 mg, 100%). Analytical method 7; t_{R}=0.43 min; [M+H]⁺=348.2.

### Example 7.5: Synthesis of methyl 4-(((1S,2S)-2-aminocyclohexyl)(methyl)amino)-4-oxo-3-((2-oxopyridin-1(2H)-yl)methyl)butanoate (AB72)

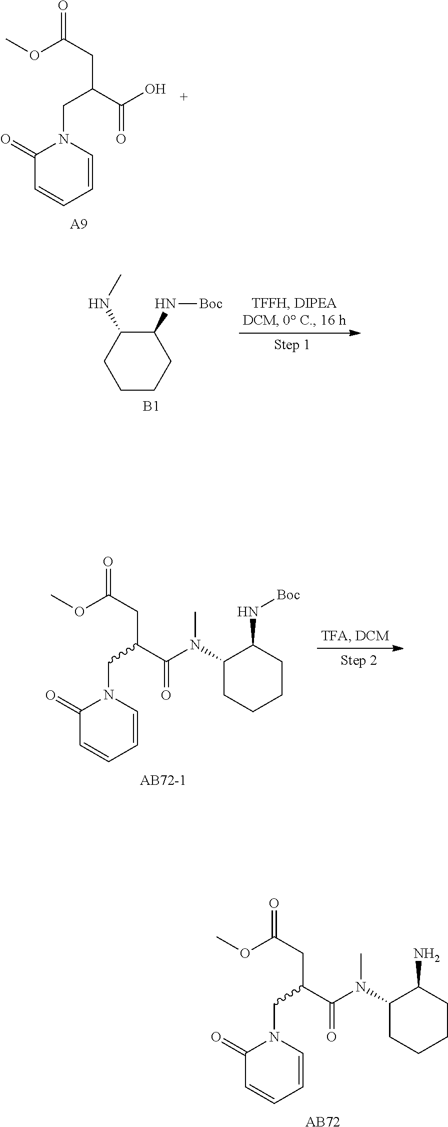

### Step 1. Methyl 4-(((1S,2S)-2-((tert-butoxycarbonyl)amino)cyclohexyl)(methyl)amino)-4-oxo-3-((2-oxopyridin-1(2H)-yl)methyl)butanoate AB72-1

AB72-1 was prepared according to the procedure in Example 7.3, Step 1 starting from B1 (419 mg, 1.84 mmol) and A9 (439 mg, 1.84 mmol) to give a mixture of two diastereomers. After workup the crude material was purified by normal phase chromatography using a silica gel column eluting with 10% DCM/MeOH to afford AB72-1 as one of the diastereomers after concentrating the pure fractions under reduced pressure (96 mg, 12% yield). Analytical method 5; t_{R}=0.86 min; [M+H]⁺=450.3.

The remaining fractions were pooled and purified by normal phase chromatography using a silica gel column eluting with 10% DCM/ACN to afford the other diastereomer after concentrating the pure fractions reduced pressure (117 mg, 14% yield). Analytical method 5; t_{R}=0.88 min; [M+H]⁺=450.3.

### Step 2. Methyl 4-(((1S,2S)-2-aminocyclohexyl)(methyl)amino)-4-oxo-3-((2-oxopyridin-1(2H)-yl)methyl)-butanoate (AB72)

AB72 (99 mg, assume quantitative yield) was prepared according to the procedure in Example 7.4, Step 2 starting from AB72-1 (96 mg, 0.21 mmol). The material was carried onto the next step without purification. Analytical method 5; t_{R}=0.59 min; [M+H]⁺=349.9.

### Example 7.6: Synthesis of tert-butyl (S)-2-(4-(aminomethyl)-3-methyl-2-oxoimidazolidin-1-yl)acetate (AB74)

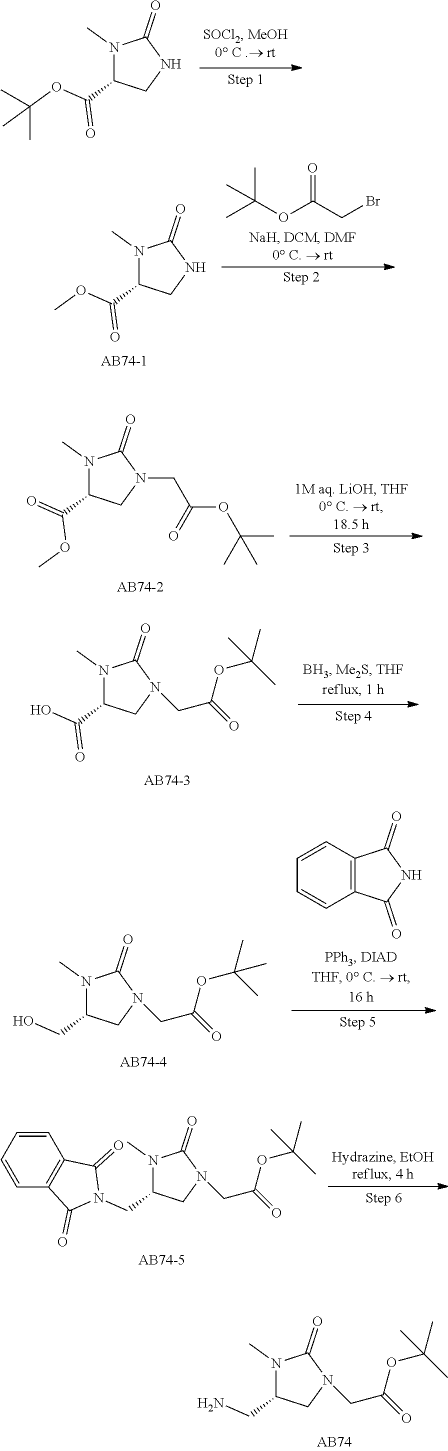

### Step 1. (R)-Methyl 3-methyl-2-oxoimidazolidine-4-carboxylate (AB74-1)

To a solution of (R)-tert-butyl 3-methyl-2-oxoimidazolidine-4-carboxylate (800 mg, 4.00 mmol) in MeOH (30 mL) at 0° C., was added thionyl chloride (0.875 mL, 11.99 mmol) dropwise. The resulting mixture was stirred at 0° C. for 3 h, then at rt for 16 h, concentrated without a heating bath, and dried under reduced pressure to afford AB74-1 (650 mg, 98%). ¹H NMR (400 MHz, CD₃OD) b ppm 4.24-4.27 (m, 1H), 3.75 (s, 3H), 3.61-3.65 (m, 1H), 3.33-3.38 (m, 1H), 2.86 (s, 3H).

### Step 2. (R)-Methyl 1-(2-(tert-butoxy)-2-oxoethyl)-3-methyl-2-oxoimidazolidine-4-carboxylate (AB74-2)

To a solution of AB74-1 in CH₂Cl₂/DMF (5:1) (24 mL) was added Cs₂CO₃ (9.98 mmol, 3.26 g). The reaction mixture was stirred at rt for 20 min, then 2-bromo-t-butylacetate (1.951 g, 10.00 mmol) was added. The resulting mixture was stirred at rt for 24 h and then diluted with EtOAc (100 mL). The organic phase washed with 10% citric acid (20 mL), 5% aq. NaHCO₃ (2×20 mL) and brine (20 mL), dried over MgSO₄, filtered, and concentrated under reduced pressure. The crude product was purified via normal phase chromatography using silica gel (100-200 mesh) column eluting with 5-35% ethyl acetate in heptane as a solvent to afford AB74-2 (1.06 g, 78%) as a thick oil. ¹H NMR (400 MHz, CDCl₃): δ ppm 4.12-4.16 (m, 1H), 4.02-3.98 (d, J=10.4 Hz, 1H), 3.80 (s, 3H), 3.76-3.78 (m, 1H), 3.74-3.78 (d, J=10.4 Hz, 1H), 3.44-3.49 (m, 1H), 2.87 (s, 3H), 1.45 (s, 9H).

### Step 3. (R)-1-(2-(tert-Butoxy)-2-oxoethyl)-3-methyl-2-oxoimidazolidine-4-carboxylic acid (AB74-3)

To a solution of AB74-2 (1060 mg, 3.89 mmol) in THF (40 mL), was added 1.0 N LiOH (6.0 mL, 6.0 mmol) in water and the resulting solution was stirred at 0° C. for 30 min, and then slowly warmed to rt and stirred at rt for 18 h. The progress of the reaction was monitored by LCMS. The reaction mixture was cooled to 0° C., neutralized to pH 7 with 1.0 N HCl (6.0 mL) and concentrated to dryness in vacuo. The obtained residue was partitioned between EtOAc (60 mL) and water (10 mL). The organic layer was dried over Na₂SO₄, filtered, and concentrated to dryness in vacuo to afford AB74-3 (916 mg, 91%) which was carried onto the next step without purification. Analytical method 7; t_{R}=0.61 min; [M−H]⁻=257.3; ¹H NMR (400 MHz, CDCl₃): δ ppm 4.12-4.16 (m, 1H), 3.94-3.98 (d, J=10.4 Hz, 1H), 3.78-3.82 (d, J=10.4 Hz, 1H), 3.76-3.78 (m, 1H), 3.54-3.57 (m, 1H), 2.92 (s, 3H), 1.47 (s, 9H).

### Step 4. (R)-tert-Butyl 2-(4-(hydroxymethyl)-3-methyl-2-oxoimidazolidin-1-yl)acetate (AB74-4)

BH₃ DMS complex (0.502 mL, 5.29 mmol) was added dropwise to a stirred solution of AB74-3 (910 mg, 3.52 mmol) in THF (50 mL) at room temperature under an atmosphere of nitrogen. The resulting mixture was heated at reflux for 1 h. The progress of the reaction was monitored by LCMS. After cooling to rt, the reaction mixture was concentrated under reduced pressure. The obtained residue was dissolved in a mixture of CH₂Cl₂ (160 mL), aqueous NaHCO₃ (10 mL), and brine (10 mL). The organic layer was dried over Na₂SO₄, filtered, and concentrated to dryness in vacuo to afford AB74-4 (700 mg, 81%) which was carried onto the next step without purification. Analytical method 16; t_{R}=0.87 min; [M-tBu]⁺=189.1. ¹H NMR (400 MHz, CD₃OD): δ ppm 3.94-3.98 (m, 2H), 3.61-3.70 (m, 2H), 3.52-3.60 (m, 3H), 2.90 (s, 3H), 1.46 (s, 9H).

### Step 5. (S)-tert-Butyl 2-(4-((1,3-dioxoisoindolin-2-yl)methyl)-3-methyl-2-oxoimidazolidin-1-yl)acetate (AB74-5)

DIAD (0.641 mL, 3.30 mmol) was added dropwise to a stirred solution of AB74-4 (700 mg, 2.87 mmol), Phthalimide (506 mg, 3.44 mmol) and triphenylphosphine (902 mg, 3.44 mmol) in THF (20 mL) under an atmosphere of nitrogen. The resulting mixture was slowly warmed to room temperature and stirred at rt for 16 h. The progress of the reaction was monitored by LCMS. The reaction mixture was concentrated under reduced pressure and the obtained residue was dissolved in a mixture of CH₂Cl₂ (100 mL) and water (20 mL). The two layers were separated and the CH₂Cl₂ layer was washed with saturated aqueous NaHCO₃ solution (20 mL) and brine (20 mL), dried over Na₂SO₄, filtered, and concentrated under reduced pressure. The crude product was purified via normal phase chromatography using a silica gel (100-200 mesh) column eluting with 5-30% ethyl acetate in heptane as a solvent to afford AB74-5 (600 mg, 56.1%). Analytical method 7; t_{R}=0.87 min; [M-tBu]⁺=318.2; ¹H NMR (400 MHz, CDCl₃): δ ppm 7.86-7.90 (d, J=6.2 Hz, 2H), 7.74-7.78 (d, J=6.2 Hz, 2H), 3.88-3.92 (m, 1H), 3.94-3.90 (d, J=10.4 Hz, 2H), 3.72-3.76 (d, J=10.4 Hz, 2H), 3.33-3.36 (t, J=8.8 Hz 1H), 2.93 (s, 3H), 1.45 (s, 9H).

### Step 6. (S)-tert-Butyl 2-(4-(aminomethyl)-3-methyl-2-oxoimidazolidin-1-yl)acetate, 2,3-dihydrophthalazine-1,4-dione (AB74)

To a well-stirred solution of AB74-5 (550 mg, 1.473 mmol) in ethanol (40 mL) was added hydrazine (0.462 mL, 14.73 mmol). The reaction mixture was heated at reflux for 4 h and then cooled. The solid formed was filtered and washed with ethanol (10 mL). The filtrates were combined and concentrated under reduced pressure. The obtained residue was dissolved in a mixture of CH₂Cl₂ (100 mL) and saturated NaHCO₃ aqueous solution (20 mL). The two layers were separated and the CH₂Cl₂ layer was washed with saturated NaHCO₃ aqueous solution (20 mL) and brine (20 mL). The organic layer was dried over Na₂SO₄, filtered, and concentrated to afford AB74 (360 mg, 80%) which was carried onto the next step without purification. Analytical method 7; t_{R}=0.45 min; [M+H]⁺=244.3; ¹H NMR (400 MHz, CDCl₃): δ ppm 7.40-8.00 (bs, 1H), 6.74-7.00 (bs, 1H), 3.90-3.94 (d, J=10.4 Hz, 1H), 3.78-3.82 (d, J=10.4 Hz, 1H), 3.50-3.54 (m, 2H), 3.30-3.34 (m, 1H), 2.94-2.99 (m, 1H), 2.80-2.86 (m, 1H), 2.80 (s, 3H), 1.46 (s, 9H).

### Example 7.7: Synthesis of 4-(((1S,2S)-2-((tert-butoxycarbonyl)amino)cyclohexyl)(methyl)amino)-4-oxobutanoic acid (AB76)

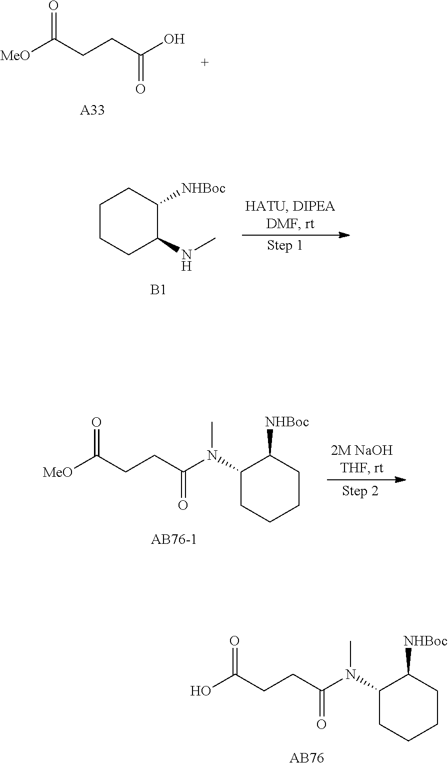

### Step 1. Methyl 4-(((1S,2S)-2-((tert-butoxycarbonyl)amino)cyclohexyl)(methyl)amino)-4-oxobutanoate (Ab76-1)

DIPEA (1.48 mL, 8.50 mmol) was added in one portion via syringe to A33 (374 mg, 2.83 mmol) in DMF (5.7 mL) in a round bottom flask charged with a magnetic stir bar. HATU (1.08 g, 2.83 mmol) was then added in one portion. After 5 min, intermediate B1 (750 mg, 2.83 mmol) was added in one portion and the resulting mixture was allowed to stir at rt under an N₂ atmosphere overnight. The reaction mixture was then diluted with EtOAc (75 mL), washed with 1 N HCl (50 mL), 1 M NaOH (50 mL), H₂O (50 mL), and brine (50 mL), dried over Na₂SO₄, filtered, and concentrated in vacuo to provide AB76-1 as a crude orange oil, which was taken on to the next step without purification. Analytical method 5; t_{R}=0.89 min; [M−H]⁻=341.3.

### Step 2. 4-(((1S,2S)-2-((tert-Butoxycarbonyl)amino)cyclohexyl)(methyl)amino)-4-oxobutanoic acid (AB76)

2 M NaOH (2.8 mL, 5.67 mmol) was added in one portion via syringe to intermediate AB76-1 (970 mg, 2.83 mmol) in THF (7.1 mL) in a round bottom flask charged with a magnetic stir bar at rt and the resulting mixture was allowed to stir at rt under a N₂ atmosphere overnight. The reaction mixture was then diluted with EtOAc (50 mL) and washed with 1 N NaOH (50 mL). The organic layer was discarded, and the aqueous layer was diluted with 1 N HCl (75 mL) and extracted with EtOAc (2×100 mL). This organic layer was washed with brine (50 mL), dried with Na₂SO₄, filtered, and concentrated in vacuo to provide a cloudy pale yellow oil. The crude oil was taken up in EtOAc (100 mL), washed with sat. NH₄Cl (50 mL), dried over Na₂SO₄, filtered, and concentrated in vacuo to provide 1.2 g of a cloudy pale yellow oil. The crude material was taken up in EtOAc and acetic acid and the sample was azeotroped with heptane to provide 650 mg (62%) of AB76 as a white solid. Analytical method 7; t_{R}=0.78 min; [M−H]⁻=327.3.

### Example 8: Building Block C—β-Amino Acids

### Example 8.1: (S)-3-((((9H-Fluoren-9-yl)methoxy)carbonyl)(methyl)amino)-4-(4-chlorophenyl)butanoic acid (C1)

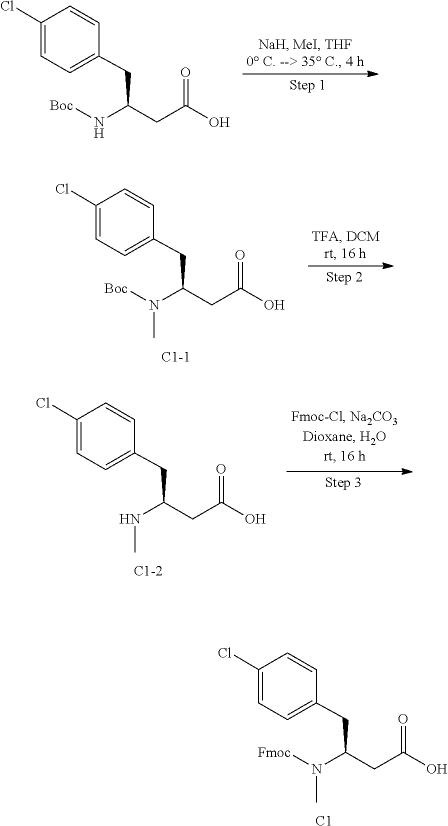

### Step 1. (S)-3-((tert-Butoxycarbonyl)(methyl)amino)-4-(4-chlorophenyl)butanoic acid (C1-1)

To a 20 L 4-necked round-bottom flask, purged and maintained with an inert atmosphere of nitrogen, containing a solution of (3S)-3-[[(tert-butoxy)carbonyl]amino]-4-(4-chlorophenyl)butanoic acid (360 g, 1.15 mol) in THF (8 L) was added by sodium hydride (212 g, 5.74 mol, 65%), in portions at 0° C. and the resulting mixture was stirred at 0° C. for 1 h. Mel was then added (1633 g, 11.5 mol) dropwise with stirring at 0° C. and the resulting solution was stirred for 4 h at 35° C. The reaction mixture was then quenched by the addition of 300 g of water/ice at −10° C., concentrated under vacuum and then diluted with 3 L of water. The aqueous phase was extracted with 3×1 L of ether. The pH value of the aqueous phase was adjusted to pH 3 with HCl (2 N) at 0° C. and the resulting solution was extracted with 3×2 L of ethyl acetate. The combined organic phases were washed with brine (1×2 L), dried over anhydrous sodium sulfate, filtered, and concentrated under vacuum to provide C1-1 as a yellow oil (284.2 g, 76%). Analytical method 7; t_{R}=1.01 min; [M+H]⁺=328.1.

### Step 2. (S)-4-(4-Chlorophenyl)-3-(methylamino)butanoic acid (C1-2)

To a 5 L 4-necked round-bottom flask, purged and maintained with an inert atmosphere of nitrogen containing a solution of C1-1 (284.2 g, 866.98 mmol) in DCM (3 L) was added TFA (990.8 g, 8.77 mol) dropwise with stirring at 0° C. The resulting solution was stirred overnight at room temperature and then concentrated under vacuum to provide intermediate C1-2 (320 g, crude). Analytical method 1; t_{R}=0.66 min; [M+H]⁺=228.2.

### Step 3. (S)-3-((((9H-Fluoren-9-yl)methoxy)carbonyl)(methyl)amino)-4-(4-chlorophenyl)butanoic acid (C1)

To a 5 L 4-necked round-bottom flask, purged and maintained with an inert atmosphere of nitrogen containing a solution of (3S)-4-(4-chlorophenyl)-3-(methylamino)butanoic acid trifluoroacetic acid salt (C1-2) (320 g crude) in dioxane:H₂O (5:1) (3.6 L) was added sodium carbonate (249.1 g, 2.35 mol) in several batches. Fmoc-Cl (242 g, 935.45 mmol) was then added in several batches at 0° C. The resulting mixture was stirred overnight at room temperature, concentrated under vacuum and then diluted with 3 L of water. The pH value of the aqueous solution was adjusted to pH 5 with HCl (1 N). The aqueous phase was extracted with 3×1 L of ethyl acetate. The combined organic phases were washed brine (1×1 L), dried over anhydrous sodium sulfate, filtered, and concentrated under vacuum. The crude residue was purified by silica gel column chromatography eluting with ethyl acetate/petroleum ether (1:5-1:3) to provide C1 as a white solid (193 g, 49.77% for two steps). Analytical method 1; t_{R}=1.60 min; [M+H]⁺=450.3. ¹H NMR (300 MHz, DMSO-d₆, ppm): δ 12.09-12.45 (br, 1H), 7.89 (m, 2H), 7.21-7.69 (m, 8H), 7.13-7.20 (m, 1H), 6.85-7.08 (br, 1H), 4.04-4.55 (m, 4H), 2.73-2.8 (m, 1H), 2.11-2.85 (m, 6H).

### Example 8.2: Synthesis of (S)-3-((((9H-fluoren-9-yl)methoxy)carbonyl)(methyl)amino)-4-(4-chloro-3-fluorophenyl)butanoic acid (C3)

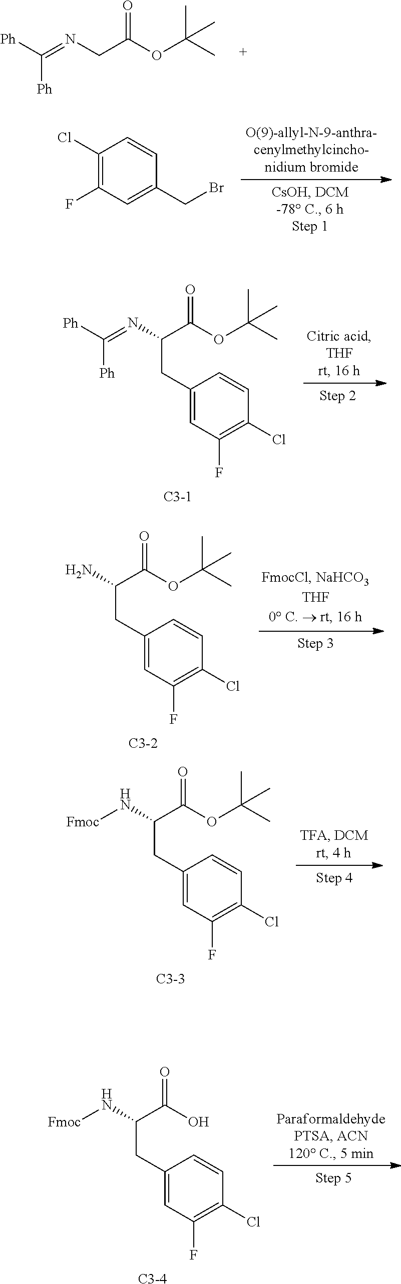

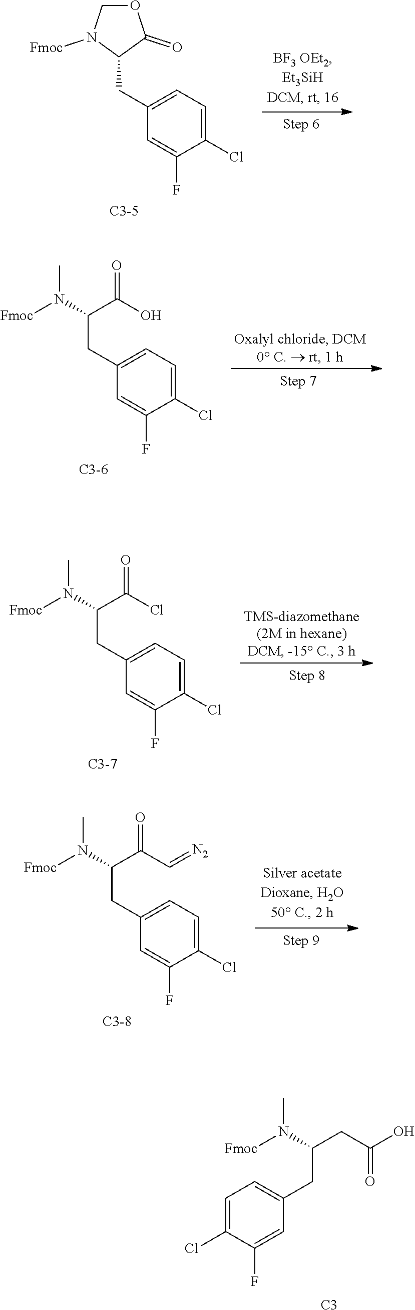

### Step 1. tert-Butyl (S)-3-(4-chloro-3-fluorophenyl)-2-((diphenylmethylene)amino)propanoate (C3-1)

A mixture of tert-butylglycinate benzophenone imine (2.5 g, 8.46 mmol) and O(9)-allyl-N-9-anthracenylmethylcinchonidium bromide (1) (513 mg, 0.846 mmol) and CsOH.H₂O (14.21 g, 85 mmol) in DCM (25 mL) was treated with a solution of 4-(bromomethyl)-1-chloro-2-fluorobenzene (3.3 g, 14.77 mmol) in DCM (5 mL) dropwise at −78° C. The resulting mixture was stirred vigorously at −78° C. for 6 h and then slowly warmed to 0° C. overnight. The obtained suspension was diluted with ether, washed with water and brine, dried over MgSO₄, filtered, and concentrated. The crude product was purified by normal phase flash chromatography (Silica gel column 40 g, eluting with 0-40% EtOAc/heptane) to give C3-1 (3.69 g, 100%) as a yellow oil. Analytical method 5; t_{R}=1.46 min; [M+H]⁺=438.2.

### Step 2. tert-Butyl (S)-2-amino-3-(4-chloro-3-fluorophenyl)propanoate (C3-2)

To a solution of C3-1 (3.69 g, 8.43 mmol) in THF (160 mL) was added citric acid (162 mL, 126 mmol) slowly. The resulting mixture was stirred at rt overnight, then cooled with ice bath and basified by K₂CO₃ (sat) slowly until basic (pH ˜9). The mixture was then diluted with EtOAc and the phases were separated. The aqueous layer was extracted with EtOAc. The combined organic phases were dried over sodium sulfate, filtered, and concentrated under reduced pressure to give C3-2 (3.63 g, 100%) which was carried onto the next step without purification. Analytical method 5: t_{R}=1.01 min; [M-OtBu]⁺=218.1.

### Step 3. (S)-tert-Butyl 2-((((9H-fluoren-9-yl)methoxy)carbonyl)amino)-3-(4-chloro-3-fluorophenyl)propanoate (C3-3)

C3-3 (2.9 g, 70.0%, white solid) was prepared according to the procedure in Example 8.1, Step 3 starting material from C3-2 (3.63 g, 8.35 mmol). Analytical method 7; t_{R}=1.40 min; [M-tBu]⁺=440.1.

### Step 4. (S)-2-((((9H-Fluoren-9-yl)methoxy)carbonyl)amino)-3-(4-chloro-3-fluorophenyl)propanoic acid (C3-4)

C3-4 (2.57 g, 100% white solid) was prepared according to the procedure in Example 8.1, Step 2 starting from C3-3 (2.9 g, 5.85 mmol). Reaction was complete in 4 h. Analytical method 7; t_{R}=1.17 min; [(M+2)/2]⁺⁺=220.1.

### Step 5. (9H-Fluoren-9-yl)methyl (S)-4-(4-chloro-3-fluorobenzyl)-5-oxooxazolidine-3-carboxylate (C3-5)

To a solution of C3-4 (620 mg, 1.41 mmol) in ACN (5 mL) was added paraformaldehyde (254 mg, 8.46 mmol) and p-toluenesulfonic acid monohydrate (26.8 mg, 0.14 mmol). The resulting mixture was heated at 120° C. for 5 min using a microwave and then concentrated under reduced pressure. The crude product was purified by normal phase flash chromatography (40 g silica gel column, eluting with 0-50% EtOAc/heptane) to give C3-5 (602 mg, 95%) as a white foam. Analytical method 7; t_{R}=1.30 min; [M+2H]²⁺=228.1.

### Step 6. (S)-2-((((9H-Fluoren-9-yl)methoxy)carbonyl)(methyl)amino)-3-(4-chloro-3-fluorophenyl)propanoic acid (C3-6)

To a solution of C3-5 (600 mg, 1.328 mmol) in DCM (8 mL) was added triethylsilane (1.591 mL, 9.96 mmol) and BF₃.OEt₂ (0.673 mL, 5.31 mmol). The reaction mixture was stirred at rt overnight and then quenched with water and extracted with DCM. The combined organic phases were dried over sodium sulfate, filtered, and concentrated. The crude product was purified by normal phase flash chromatography (silica gel column 40 g, eluting with 30-100% EtOAc/heptane) to give C3-6 (521 mg, 86%) as a white foam. Analytical method 7; t_{R}=1.22 min; [M+Na]⁺=476.2.

### Step 7. (S)-(9H-Fluoren-9-yl)methyl (1-chloro-3-(4-chloro-3-fluorophenyl)-1-oxopropan-2-yl)(methyl)carbamate, (S)-methyl 2-((((9H-fluoren-9-yl)methoxy)carbonyl)(methyl)amino)-3-(4-chloro-3-fluorophenyl)propanoate (C3-7)

To a solution of C3-6 (521 mg, 1.148 mmol) in DCM (6 mL) was added 2 drops of DMF and oxalyl chloride (0.201 mL, 2.296 mmol) was added dropwise at 0° C. The reaction mixture was stirred at rt for 1 h and then concentrated to dryness to afford C3-7 (542 mg, 100%) as a light yellow foam. Analytical method 7; t_{R}=1.32 min; [M-Cl+OCH₃+H]⁺=467.9.

### Step 8. (S)-(9H-Fluoren-9-yl)methyl (1-(4-chloro-3-fluorophenyl)-4-diazo-3-oxobutan-2-yl)(methyl)carbamate (C3-8)

A solution of C3-7 (542 mg, 1.148 mmol) in DCM (5 mL) was added to TMS-diazomethane (2M in hexane) (1.722 mL, 3.44 mmol) dropwise at −20 to −10° C. The reaction mixture was stirred for 3 h and then quenched with 10% HOAc (10 mL). The organic layer was separated, dried over Na₂SO₄, filtered, and concentrated. The obtained residue was redissolved in EtOAc, washed with Na₂CO₃ (aq), dried over Na₂SO₄, filtered, and concentrated to afford C3-8 (549 mg, 100%) as a light yellow foam which was carried onto the next step without purification. Analytical method 7; t_{R}=1.30 min; [M-N₂+H]⁺=450.1.

### Step 9. (S)-3-((((9H-fluoren-9-yl)methoxy)carbonyl)(methyl)amino)-4-(4-chloro-3-fluorophenyl)butanoic acid (C3)

To a solution of C3-8 (549 mg, 1.148 mmol) in dioxane (10 mL) and H₂O (2.5 mL) was added silver acetate (19.16 mg, 0.115 mmol). The reaction mixture was heated to 50° C. for 2 h and then diluted with DCM and H₂O. The aqueous layer was adjusted to pH 1 using HCl (4 N). The resulting mixture was filtered through Celite®. The organic layer was separated, dried over Na₂SO₄, filtered, and concentrated. The crude product was purified by normal phase flash chromatography (silica gel 24 g column, eluting with 10-100% EtOAc/heptane) to give C3 (351 mg, 65.3%). Analytical method 7; t_{R}=1.20 min; [M+H]⁺=467.8.

TABLE 22
β-Amino acids

| | | | Synthesis |
|---|---|---|---|
| BB | | | procedure/ |
| No. | Structure | Compound Name | LCMS |
| C2 | 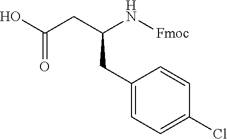 | (S)-3-((((9H-fluoren-9- yl)methoxy)carbonyl)amino)- 4-(4-chlorophenyl)butanoic acid | Commercially Available |
| C4 | 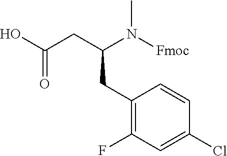 | (S)-3-((((9H-fluoren-9- yl)methoxy)carbonyl)(methyl) amino)-4-(4-chloro-2- fluorophenyl)butanoic acid | C3 (Example 8.2)- starting from 4-chloro- 2-fluorobenzylbromide/ Analytical method 7 t_{R} = 1.21 min [M + H]⁺ = 467.8 |

### Example 9: Building Block D (BB D)—α-Amino Acid

### Example 9.1: Synthesis of (S)-2-((((9H-fluoren-9-yl)methoxy)carbonyl)amino)-4-fluorobutanoic acid (D2)

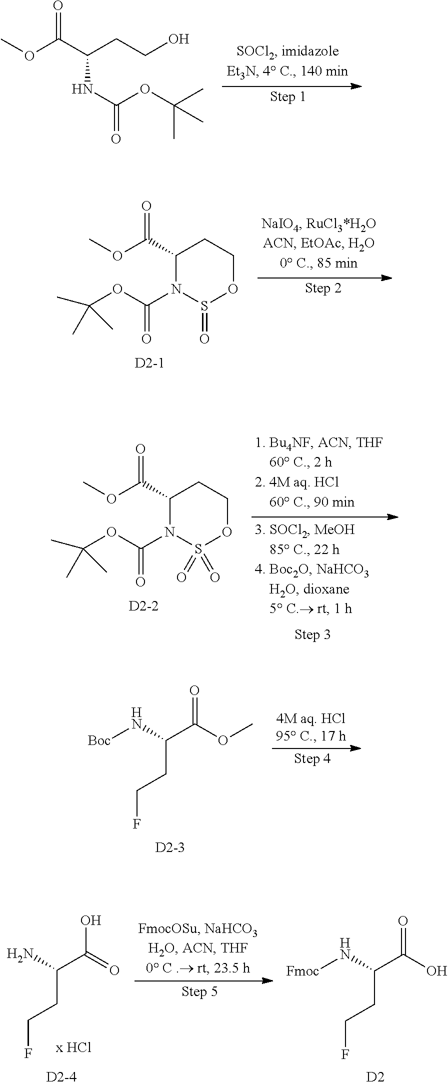

### Step 1. 3-(tert-Butyl) 4-methyl (4S)-1,2,3-oxathiazinane-3,4-dicarboxylate 2-oxide (D2-1)

To a solution of imidazole (173.4 g, 2.55 mol) and triethylamine (132.0 mL, 939 mmol) in DCM (3 L) at 4° C. was added SOCl₂ (65 mL) dropwise over 1 h. The resulting suspension was stirred for 25 min at 4° C., and then a solution of methyl (tert-butoxycarbonyl)-L-homoserinate (110.65 g, 90% content, 427.0 mmol) in DCM (0.5 L) was added over 30 min. The reaction mixture was stirred at 4° C. for 2 h 20 min and then quenched by addition of H₂O (1.2 L). The phases were separated and the aqueous layer was extracted with DCM (0.5 L). The combined organic phases were washed with H₂O (1 L) and brine (1 L), dried over Na₂SO₄, filtered, and concentrated to dryness in vacuo. The crude product was purified by silica gel chromatography. D2-1 was obtained as a yellow oil (78.14 g, 279.8 mmol, 66% yield).

### Step 2. 3-(tert-Butyl) 4-methyl (4S)-1,2,3-oxathiazinane-3,4-dicarboxylate 2-oxide (D2-2)

To a solution of D2-1 (97.3 g, 348.4 mmol) in ACN (1.8 L) and EtOAc (180 mL) at 0° C. was added RuCl₃H₂O (4.19 g, 20.2 mmol), followed by a cooled cloudy solution of NaIO₄ (149.0 g, 696.7 mmol) in H₂O (800 mL) over 15 min. The reaction mixture was stirred for 85 min at 0° C. and then Et₂O (1400 mL) was added. The resulting suspension was filtered and the phases were separated. The aqueous phases were extracted with Et₂O (2×800 mL). The combined organic phases were washed with NaHCO₃-solution (1000 mL) and brine, dried over Na₂SO₄, filtered, and concentrated to dryness in vacuo to afford D2-2 (92.75 g, 282.7 mmol, 90% purity by NMR, 81% yield) as a white solid. The crude product was onto the next step without purification

### Step 3. Methyl (S)-2-((tert-butoxycarbonyl)amino)-4-fluorobutanoate (D2-3)

To D2-2 (92.75 g, 90% purity, 282.7 mmol) dissolved in ACN (1 L) was added 1 M Tetrabutylammoniumfluoride in THF (466.5 mL; 466.5 mmol) slowly at rt. The reaction mixture was stirred for 2 h at 60° C., then the solvent was evaporated in vacuo, and the obtained oil was mixed with 4 M aq. HCl (800 mL). The resulting mixture was heated to 60° C. and stirred for 90 min and then concentrated to dryness in vacuo. The obtained oil was co-evaporated with toluene to dryness to give a viscous yellow oil

The crude intermediate was dissolved in MeOH (1200 mL) and SOCl₂ (102.6 mL, 1.41 mol) was added at 5° C. and the resulting mixture was stirred under reflux for 22 h. After complete consumption of starting material, the solvent was removed in vacuo and the crude product was co-evaporated with toluene to dryness.

The obtained oil was dissolved in 1 M aq. NaHCO₃ and dioxane (200 mL) at 5° C. and a solution of Boc₂O (124.1 g, 568.6 mmol) in dioxane (100 mL) was added. The reaction mixture was allowed to warm to rt and then stirred for 21 h. H₂O (500 mL) and Et₂O (500 mL) were added and the phases were separated. The aqueous phase was extracted with Et₂O (2×500 mL). The combined organic phases were washed with H₂O (500 mL) and brine (300 mL), dried over Na₂SO₄, filtered, and concentrated to dryness in vacuo. The crude product was purified by silica gel chromatography to provide D2-3 (55.0 g, 233.8 mmol, 83% yield) as a yellow oil.

### Step 4. (S)-2-Amino-4-fluorobutanoic acid (D2-4)

D2-3 (55.0 g, 233.8 mmol) was dissolved in 4 M HCl (1000 mL) and the resulting mixture was stirred for 17 h at 95° C. and then concentrated in vacuo. Further drying was achieved by co-evaporation with toluene. D2-4 (33.94 g, 194 mmol, 90% purity, 83% yield) was obtained as a yellow solid.

### Step 5. (S)-2-((((9H-Fluoren-9-yl)methoxy)carbonyl)amino)-4-fluorobutanoic acid (D2)

To D2-4 (33.9 g, 194 mmol) dissolved in 0.5 M aq. NaHCO₃ (1 L, 500 mmol) was added ACN/THF (4:1). The reaction mixture was cooled to 0° C. and a solution of Fmoc-OSu (75.35 g, 223.4 mmol) in ACN/THF (4:1) (500 mL) was added. The resulting mixture was allowed to warm to rt and stirred for 23.5 h. Ice (1000 mL) was added and the reaction mixture was carefully acidified to pH of ˜2-3 by the addition of conc. HCl (25 mL). Et₂O (2 L) was added and the layers were separated. The aqueous phase was extracted with Et₂O (600 mL). The combined organic phases were washed with H₂O (600 mL) and brine, dried over Na₂SO₄, filtered, and concentrated to dryness in vacuo. The crude product was purified by silica gel chromatography. Pure fractions were combined and concentrated to dryness in vacuo. The residue was suspended in toluene (150 mL) and then concentrated to dryness in vacuo. This step was repeated twice. D2 (17.5 g, 50.7 mmol, 26% yield) was obtained as an off-white solid. Analytical method 10; t_{R}=0.96 min; [M+H]⁺=344.1. ¹H NMR (600 MHz, DMSO-d₆) δ 12.75 (s, 1H), 7.88 (d, J=7.5 Hz, 2H), 7.71 (t, J=7.2 Hz, 2H), 7.40 (t, J=7.4 Hz, 2H), 7.32 (t, J=7.4 Hz, 2H), 4.55 (dt, J=9.7, 5.0 Hz, 0.5H), 4.48 (dt, J=9.7, 4.8 Hz, 1H), 4.40 (td, J=8.9, 4.4 Hz, 0.5H), 4.30 (d, J=7.1 Hz, 2H), 4.22 (t, J=7.0 Hz, 1H), 4.06 (td, J=9.6, 4.4 Hz, 1H), 2.14 (dtt, J=19.0, 8.7, 4.4 Hz, 1H), 1.92 (dtd, J=31.2, 10.2, 5.1 Hz, 1H).

### Example 9.2: Synthesis of (S)-2-((((9H-fluoren-9-yl)methoxy)carbonyl)amino)-2-(oxetan-3-yl)acetic acid (D4)

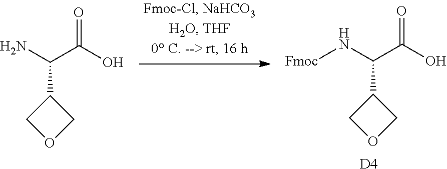

To a solution of (S)-2-amino-2-(oxetan-3-yl)acetic acid (1 g, 7.63 mmol) in THF (40 mL) was added NaHCO₃ (19.07 ml, 38.1 mmol) at 0° C., followed by FmocCl (1.973 g, 7.63 mmol) in THF (5 mL). The resulting mixture was slowly warmed to rt and stirred overnight. The reaction mixture was concentrated under vacuum and diluted with water (100 mL). The aqueous phase was washed with ethyl ether:heptane (3:2, 150:100 mL), then was acidified to pH 1-2 using HCl (4 M in dioxane) at 0° C., and extracted with EtOAc (2×100 mL). The combined organic phases were dried over Na₂SO₄. filtered, and concentrated to yield D4 (1.82 g, 67.5%) as a white solid. Analytical method 7; t_{R}=0.91 min; [M+H]⁺=354.1.

### Example 9.3: Synthesis of N-(((9H-fluoren-9-yl)methoxy)carbonyl)-O-(difluoromethyl)-L-serine (D12)

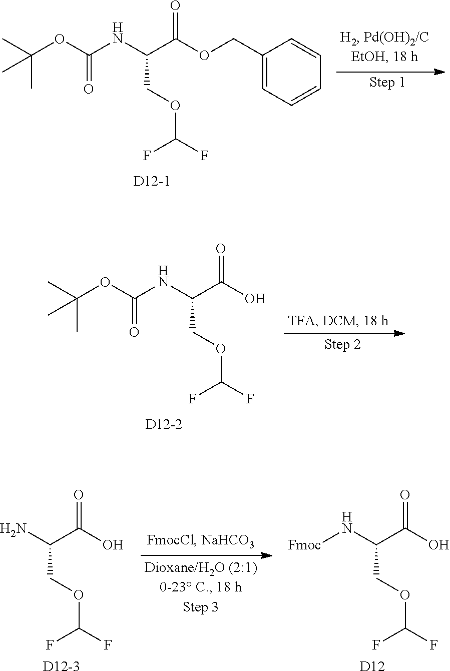

### Step 1. N-(tert-Butoxycarbonyl)-O-(difluoromethyl)-L-serine (D12-2)

To D12-1 (prepared according to the procedure in patent: US2015/218212 A1) (1 g, 2.90 mmol) in EtOH (20 mL) in a round bottom flask evacuated using vacuum and filled with nitrogen gas three times was added 20% palladium hydroxide on carbon (contains 50% water) (0.203 g, 0.290 mmol). The reaction flask was again evacuated using vacuum and filled with nitrogen gas three times and then filled with hydrogen in a balloon through a glass connector. The reaction mixture was stirred overnight and monitored by LCMS. The hydrogen balloon was then disconnected and the reaction flask was vacuumed and filled with nitrogen three times before being exposed to air. Celite® and 20 mL of DCM were added. The mixture was filtered through a pad of pre-wetted (with DCM) Celite® and washed with additional DCM. The filtrate was concentrated via rotovap to afford D12-2 as a yellow oil, which was carried onto the next step without purification. Analytical method 5; t_{R}=0.94 min; observed mass 243.3 (calculated mass not observed).

### Step 2. O-(Difluoromethyl)-L-serine (D12-3)

To a solution of D12-2 (1.56 g, 6.11 mmol) in anhydrous DCM (20 mL) was added TFA (7.06 mL, 92 mmol). The resulting mixture was stirred at rt overnight, after which LCMS showed complete consumption of starting material. The reaction mixture was concentrated via rotovap, diluted with toluene (10 mL and concentrated again (repeated this process twice) to afford D12-3 (0.948 g, 6.11 mmol, 100% yield), which was carried onto the next step without purification. Analytical method 5; t_{R}=0.13 min; [M+H]⁺=156.0.

### Step 3. N-(((9H-Fluoren-9-yl)methoxy)carbonyl)-O-(difluoromethyl)-L-serine (D12)

To D12-3 (0.948 g, 6.11 mmol) dissolved in a mixture of 1,4-dioxane (40 mL) and water (20.00 mL) was added sat. NaHCO₃ (30.8 g, 36.7 mmol) at 0° C., followed by Fmoc-Cl (1.739 g, 6.72 mmol). The resulting mixture was stirred at 0° C. for one hour, then at rt overnight, and then EtOAc was added. The organic phase was washed with H₂O, 1 N HCl and brine, dried over Na₂SO₄, and concentrated in vacuo. The crude product was purified by silica gel flash chromatography (eluting with 0-100% EtOAc:heptane) to give pure D12 (1.6 g, 4.24 mmol, 69.4% yield) a pure white solid after being dried under reduced pressure overnight. Analytical method 5; t_{R}=0.63 min; [M+H]⁺=378.1.

TABLE 23
Building block D - α-amino acids

| | | Synthesis |
|---|---|---|
| | | procedure/ |
| BB | Structure/Chemical | LCMS |
| D1 | 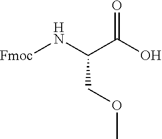 N-(((9H-fluoren-9- yl)methoxy)carbonyl)- O-methyl-L-serine | Commercially Available |
| D3 | 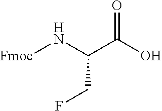 (R)-2-((((9H-fluoren-9- yl)methoxy)carbonyl) amino)-3- fluoropropanoic acid | Commercially Available |
| D5 | 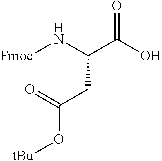 (S)-2-((((9H-fluoren-9- yl)methoxy)carbonyl) amino)-4-(tert-butoxy)-4- oxobutanoic acid | Commercially Available |
| D6 | 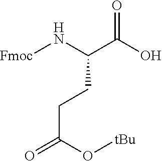 (S)-2-((((9H-fluoren-9- yl)methoxy)carbonyl) amino)-5-(tert-butoxy)-5- oxopentanoic acid | Commercially Available |
| D7 | 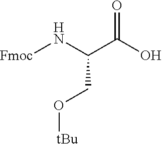 N-(((9H-fluoren-9- yl)methoxy)carbonyl)- O-(tert-butyl)-L-serine | commercially available |
| D8 | 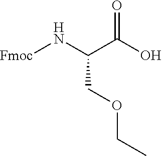 N-(((9H-fluoren-9- yl)methoxy)carbonyl)- O-ethyl-L-serine | commercially available |
| D9 | 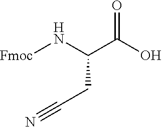 (S)-2-((((9H-fluoren-9- yl)methoxy)carbonyl) amino)-3- cyanopropanoic acid | commercially available |
| D10 | 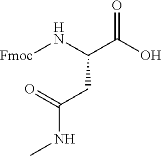 N2-(((9H-fluoren-9- yl)methoxy)carbonyl)- N4-methyl-L- asparagine | commercially available |
| D11 | 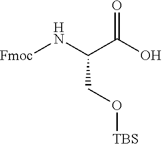 N-(((9H-fluoren-9- yl)methoxy)carbonyl)- O-(tert- butyldimethylsilyl)-L- serine | commercially available |
| D13 | 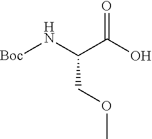 N-(tert- butoxycarbonyl)-O- methyl-L-serine | commercially available |

### Example 10: Building Block E—α-Amino Acid

### Example 10.1: Synthesis of (S)-3-(azetidin-1-yl)-2-((tert-butoxycarbonyl)amino)propanoic acid (E10)

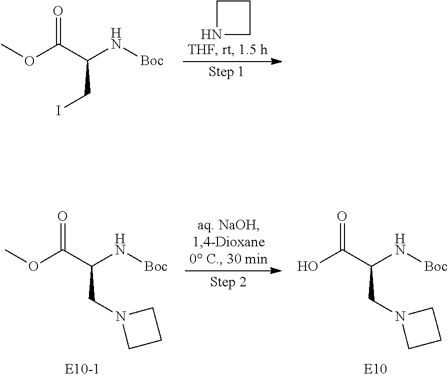

### Step 1. Methyl (S)-3-(azetidin-1-yl)-2-((tert-butoxycarbonyl)amino)propanoate (E10-1)

To a solution of (R)-methyl 2-((tert-butoxycarbonyl)amino)-3-iodopropanoate (500 mg, 1.519 mmol) in THF (5 mL) was added azetidine (347 mg, 6.08 mmol). The resulting mixture was stirred at rt for 1.5 h, after which it was diluted with sat. NaHCO₃ (10 mL) and extracted with 2×20 mL of EtOAc. The combined organic layers were combined, dried over sodium sulfate, concentrated via rotovap, and purified by silica gel flash chromatography (40 g column, eluting with 0-10% MeOH in DCM) to afford E10-1 (360 mg, 1.394 mmol, 92% yield) as a light yellow oil. Analytical method 5; t_{R}=0.81 min; [M+H]⁺=259.2.

### Step 2. (S)-3-(Azetidin-1-yl)-2-((tert-butoxycarbonyl)amino)propanoic acid (E10)

To E10-1 (350 mg, 1.355 mmol) in dioxane (2 mL) cooled to 0° C. in an ice bath was added 0.67 M NaOH (5.06 mL, 3.39 mmol) dropwise via a syringe. The resulting mixture was stirred at 0° C. for 30 min (LCMS showed complete consumption of starting material after 15 min) and then acidified with 0.2 M HCl to pH 5. The aqueous phase was extracted with EtOAc but the product remained in aqueous layer. The aqueous layer was lyophilized overnight and MeOH was added to dissolve the desired product. The mixture was sonicated and filtered. The filtrate was concentrated via rotovap to afford E10 (370 mg, 1.515 mmol, 112% yield). Analytical method 5; t_{R}=0.30 min; [M−H]⁻=243.4.

### Example 10.2: Synthesis of (S)-2-amino-5-morpholinopentanoic acid (E20)

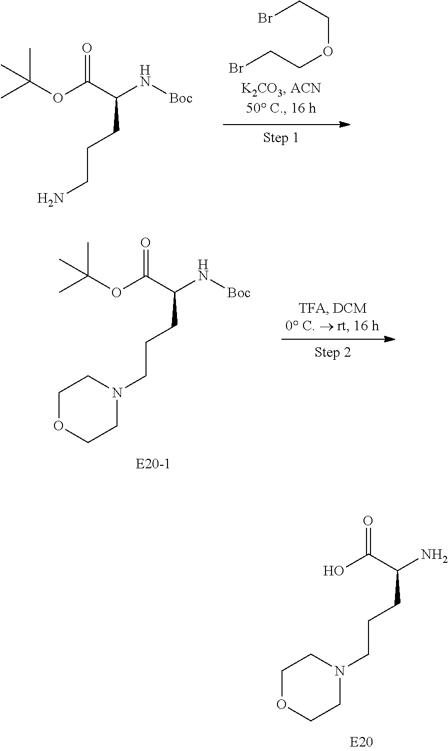 [SMILES: CC(C)(C)OC(=O)N[C@@H](CCCN)C(=O)OC(C)(C)C ; BrCCOCCBr ; CC(C)(C)OC(=O)N[C@@H](CCCN1CCOCC1)C(=O)OC(C)(C)C ; N[C@@H](CCCN1CCOCC1)C(=O)O]

### Step 1. tert-Butyl (S)-2-((tert-butoxycarbonyl)amino)-5-morpholinopentanoate (E20-1)

To a solution of (S)-tert-butyl 5-amino-2-((tert-butoxycarbonyl)amino)pentanoate (500 mg, 1.734 mmol) and bis-2-Bromoethylether (483 mg, 2.081 mmol) in dry ACN (6 mL) was added a solution of K₂CO₃ (719 mg, 5.20 mmol) in dry ACN (2 mL). The resulting mixture was stirred overnight at 50° C., then concentrated in vacuo and the obtained residue was directly purified by normal phase silica gel chromatography using a silica gel column eluting with EtOAc in heptane 10-70% to yield E20-1 (500 mg, 80%) as a white solid. Analytical method 5; t_{R}=1.00 min; [M+H]⁺=358.9.

### Step 2. (S)-2-Amino-5-morpholinopentanoic acid (E20)

To a solution of E20-1 (220 mg, 0.614 mmol) in dry DCM (2 mL) at 0° C. was slowly added trifluoroacetic acid (1 mL, 12.98 mmol) dropwise. The resulting mixture was stirred at rt for 3 h, then concentrated without heat, and the obtained residue was dried under high vacuum for 2 h. The residue E20 (210 mg, 97%) was used as is in the next step without purification. Analytical method 7; t_{R}=0.12 min; [M+H]⁺=203.0.

### Example 10.3: Synthesis of (S)-tert-butyl 2-amino-5-(3,3,4,4-tetrafluoropyrrolidin-1-yl)pentanoate (E23)

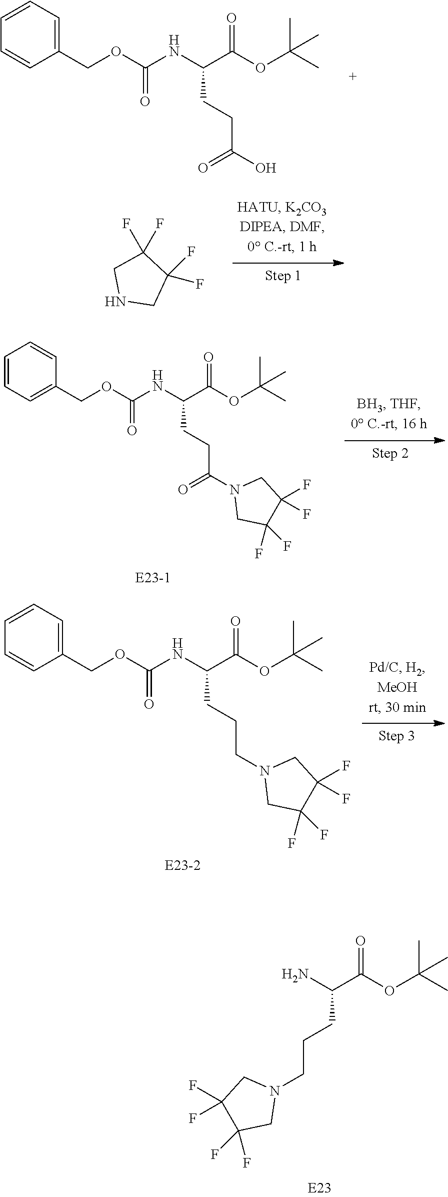 [SMILES: CC(C)(C)OC(=O)[C@H](CCC(=O)O)NC(=O)OCc1ccccc1 ; FC1(F)CNCC1(F)F ; CC(C)(C)OC(=O)[C@H](CCC(=O)N1CC(F)(F)C(F)(F)C1)NC(=O)OCc1ccccc1 ; CC(C)(C)OC(=O)[C@H](CCCN1CC(F)(F)C(F)(F)C1)NC(=O)OCc1ccccc1 ; CC(C)(C)OC(=O)[C@@H](N)CCCN1CC(F)(F)C(F)(F)C1]

### Step 1. (S)-tert-Butyl 2-(((benzyloxy)carbonyl)amino)-5-oxo-5-(3,3,4,4-tetrafluoropyrrolidin-1-yl)pentanoate (E23-1)

To a solution of (S)-4-(((benzyloxy)carbonyl)amino)-5-(tert-butoxy)-5-oxopentanoic acid (1 g, 2.96 mmol) in DMF (5 mL) at 0° C. was added DIPEA (2.59 mL, 14.82 mmol) and HATU (1.240 g, 3.26 mmol). The resulting mixture was stirred at room temperature for 15 min and then 3,3,4,4-tetrafluoropyrrolidine (0.532 g, 2.96 mmol) (pre-basified in DMF (5 mL) by excess K₂CO₃ (1.229 g, 8.89 mmol) for 20 min) was added. The reaction mixture was stirred at room temperature for 1 h, then filtered and purified by chromatography (80 g silica gel column, eluting with 20-50% EtOAc/Heptane) to yield E23-1 (1.22 g, 89%). ¹H NMR (400 MHz, chloroform-d) δ 7.38 (s, 5H), 5.46 (s, 1H), 5.12 (d, J=8.9 Hz, 2H), 4.27 (s, 1H), 3.92 (dd, J=30.6, 15.5 Hz, 4H), 2.29 (s, 3H), 1.96 (s, 1H), 1.48 (s, 9H).

### Step 2. (S)-tert-Butyl 2-(((benzyloxy)carbonyl)amino)-5-(3,3,4,4-tetrafluoropyrrolidin-1-yl)pentanoate, (S)-tert-butyl 2-amino-5-(3,3,4,4-tetrafluoropyrrolidin-1-yl)pentanoate (E23-2)

To a solution of E23-1 (1.02 g, 2.206 mmol) in THF (10 mL) at 0° C. was added borane (2 M in THF, 4.41 mL, 4.41 mmol) dropwise. The resulting mixture was gradually warmed to room temperature and stirred at 22° C. for 16 h. The reaction mixture was quenched dropwise with MeOH (10 mL) at −78° C. and evaporated. The process was repeated 3 more times. EtOAc (10 mL) was added to the mixture and the solvent was evaporated. The obtained residue was purified by chromatography (80 g silica gel column, eluting with 10-50% EtOAc/Heptane) to afford product E23-2 (0.63 g, 64% yield). ¹H NMR (400 MHz, Chloroform-d) δ 7.38 (s, 5H), 5.43 (s, 1H), 5.13 (d, J=5.8 Hz, 2H), 4.26 (s, 1H), 3.10 (t, J=12.4 Hz, 4H), 2.59 (s, 2H), 2.19 (s, 2H), 1.87 (s, 1H), 1.69 (d, J=13.8 Hz, 1H), 1.48 (s, 9H).

### Step 3. (S)-tert-butyl 2-amino-5-(3,3,4,4-tetrafluoropyrrolidin-1-yl)pentanoate (E23)

To a solution of E23-2 (0.63 g, 1.405 mmol) in MeOH (5 mL) in a flask was added Pd/C (0.020 g, 0.140 mmol). The flask was flushed with hydrogen three times and stirred under an atmosphere of hydrogen using a balloon for 30 min. The reaction mixture was flushed with nitrogen 3 times, and then filtered through Celite®-filled filtration funnel. The filtrate was concentrated and dried under vacuum to yield E23 (442 mg, crude). Analytical method 5; t_{R}=0.97 min; [M+H]⁺=315.2. The residue was used in the next step without purification.

### Example 10.4: Synthesis of N⁴,N⁴-dimethyl-L-asparagine trifluoroacetate (E21)

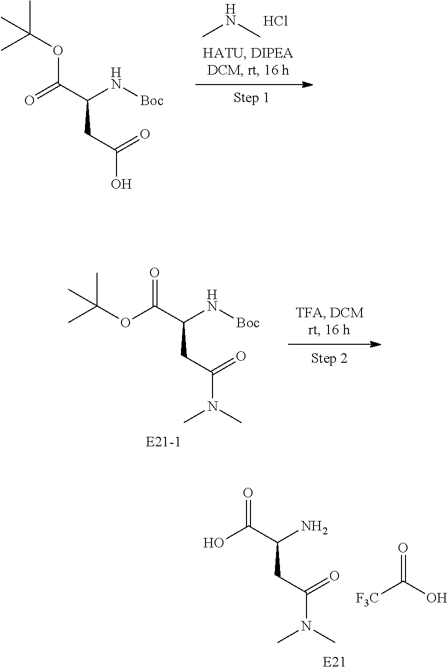

### Step 1. tert-Butyl N²-(tert-butoxycarbonyl)-N⁴,N⁴-dimethyl-L-asparaginate (E21-1)

To a solution of (S)-4-(tert-butoxy)-3-((tert-butoxycarbonyl)amino)-4-oxobutanoic acid (1.000 g, 3.46 mmol) in anhydrous DCM (30 ml) was added dimethylamine HCl salt (0.846 g, 10.37 mmol), HATU (1.511 g, 3.97 mmol) and DIPEA (1.811 ml, 10.37 mmol). The reaction mixture was stirred at rt for 16 h and then concentrated. The crude product was purified by silica gel chromatography (eluting with heptane/EtOAc 95/5 to 40/60) to afford E21-1 (1.10 g, 96%). Analytical method 7; t_{R}=0.89 min; [M+H]⁺=317.1.

### Step 2. N⁴,N⁴-Dimethyl-L-asparagine trifluoroacetate (E21)

To a solution of E21-1 (350 mg, 1.106 mmol) in DCM (6 mL) at 0° C. was added slowly TFA (1.5 mL, 19.47 mmol). The reaction mixture was stirred for 16 h at rt and then concentrated to dryness in vacuo to afford E21 (300 mg, 94%). Analytical method 16; t_{R}=0.13 min; [M+H]⁺=160.8.

### Example 10.5: Synthesis of (S)-2-amino-5-(pyrrolidin-1-yl)pentanoic acid trifluoroacetate (E22)

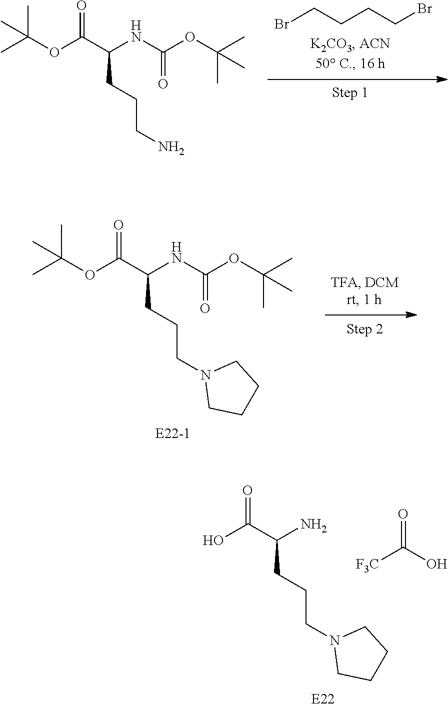

### Step 1. tert-Butyl (S)-2-((tert-butoxycarbonyl)amino)-5-(pyrrolidin-1-yl)pentanoate (E22-1)

To a solution of tert-butyl (S)-5-amino-2-((tert-butoxycarbonyl)amino)pentanoate (1.6 g, 5.55 mmol) and 1,4-dibromobutane (2.396 g, 11.10 mmol) in 6 mL of anhydrous ACN was added a solution of K₂CO₃ (2.300 g, 16.64 mmol) in 2 mL of anhydrous ACN. The resulting mixture was stirred at rt for 30 min before being heated to 50° C. and stirred overnight. The reaction mixture was filtered through a pad of Celite® and concentrated. The crude residue was purified via silica gel chromatography (eluted by DCM/MeOH (TEA modifier) 95/5 to 80/20) to afford white solid intermediate E22-1 (700 mg, 37%). Analytical method 7; t_{R}=1.18 min; [M+H]+=343.3.

### Step 2. (S)-2-Amino-5-(pyrrolidin-1-yl)pentanoic acid trifluoroacetate (E22)

To a solution of E22-1 (700 mg, 2.044 mmol) in anhydrous DCM (8 mL) at 0° C., was added TFA (2.0 ml, 25.96 mmol) dropwise at 0° C. The reaction was stirred at rt for 3 h, after which it was concentrated in vacuo. The crude residue was dried under reduced pressure to afford E22 (940 mg, 100%) as an off-white solid. Analytical method 7; t_{R}=0.30 min; [M+H]⁺=187.2.

TABLE 24
Building block E

| | | Synthesis |
|---|---|---|
| BB | | procedure/ |
| No. | Structure | LCMS |
| E1 | 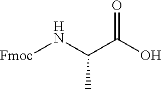 (((9H-fluoren-9- yl)methoxy)carbonyl)-L- alanine | commercially available |
| E2 | 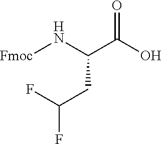 (S)-2-((((9H-fluoren-9- yl)methoxy)carbonyl) amino)-4,4- difluorobutanoic acid | commercially available |
| E3 | 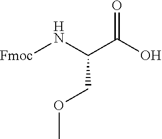 N-(((9H-fluoren-9- yl)methoxy)carbonyl)- O-methyl-L-serine | commercially available |
| E4 | 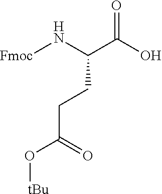 (S)-2-((((9H-fluoren-9- yl)methoxy)carbonyl) amino)-5-(tert-butoxy)-5- oxopentanoic acid | commercially available |
| E5 | 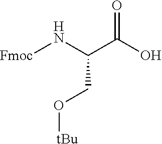 N-(((9H-fluoren-9- yl)methoxy)carbonyl)- O-(tert-butyl)-L-serine | commercially available |
| E6 | 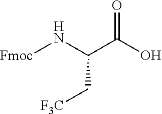 (S)-2-((((9H-fluoren-9- yl)methoxy)carbonyl) amino)-4,4,4- trifluorobutanoic acid | commercially available |
| E7 | 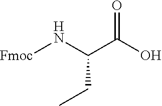 (S)-2-((((9H-fluoren-9- yl)methoxy)carbonyl) amino)butanoic acid | commercially available |
| E8 | 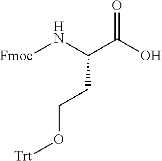 N-(((9H-fluoren-9- yl)methoxy)carbonyl)- O-trityl-L-homoserine | commercially available |
| E11 | 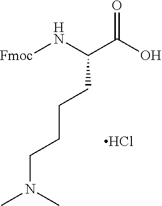 N2-(((9H-fluoren-9- yl)methoxy)carbonyl)- N6,N6-dimethyl-L- lysine hydrochloride | commercially available |
| E12 | 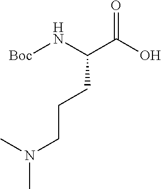 [SMILES: CN(C)CCC[C@H](NC(=O)OC(C)(C)C)C(=O)O] (S)-2-((tert- butoxycarbonyl)amino)- 5-(dimethylamino) pentanoic acid | commercially available |
| E13 | 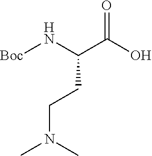 [SMILES: CN(C)CC[C@H](NC(=O)OC(C)(C)C)C(=O)O] (S)-2-((tert- butoxycarbonyl)amino)- 4-(dimethylamino) butanoic acid | commercially available |
| E14 | 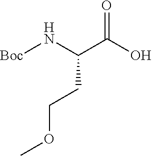 [SMILES: COCC[C@H](NC(=O)OC(C)(C)C)C(=O)O] N-(tert-butoxycarbonyl)- O-methyl-L-homoserine | commercially available |
| E15 | 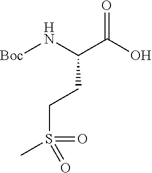 [SMILES: CC(C)(C)OC(=O)N[C@@H](CCS(C)(=O)=O)C(=O)O] (S)-2-((tert- butoxycarbonyl)amino)-4- (methylsulfonyl)butanoic acid | commercially available |
| E16 | 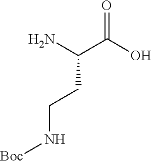 [SMILES: CC(C)(C)OC(=O)NCC[C@H](N)C(=O)O] (S)-2-amino-4-((tert- butoxycarbonyl)amino) butanoic acid | commercially available |
| E17 | 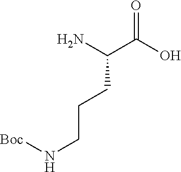 [SMILES: CC(C)(C)OC(=O)NCCC[C@H](N)C(=O)O] (S)-2-amino-5-((tert- butoxycarbonyl)amino) pentanoic acid | commercially available |
| E18 | 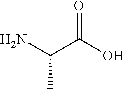 L-alanine | commercially available |
| E19 | 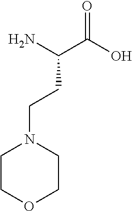 [SMILES: N[C@@H](CCN1CCOCC1)C(=O)O] (S)-2-amino-4- morpholinobutanoic acid | commercially available |
| E24 | 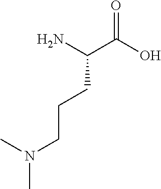 (S)-2-amino-5- (dimethylamino)pentanoic acid | commercially available |
| E25 | 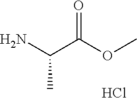 methyl L-alaninate hydrochloride | commercially available |
| E26 | 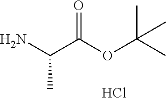 tert-butyl L-alaninate hydrochloride | commercially available |

### Example 11: Building Block F—Aldehydes

### Example 11.1: Synthesis of 4-chloro-2-(4-(2-((dimethylamino)methyl)-1-methyl-1H-imidazol-5-yl)phenoxy)-benzaldehyde (F1)

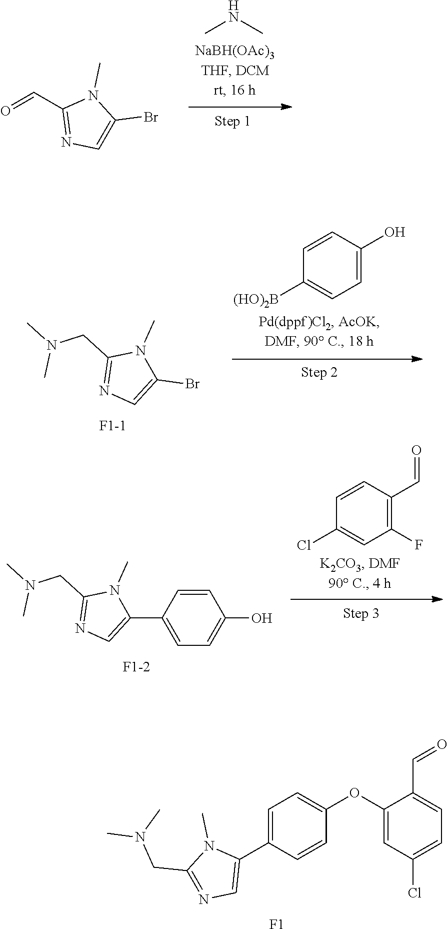

### Step 1. [(5-Bromo-1-methyl-1H-imidazol-2-yl)methyl]dimethylamine (F1-1)

Into a 5-L 3-necked round-bottom flask, purged and maintained under an inert atmosphere of nitrogen, were placed 5-bromo-1-methyl-1H-imidazole-2-carbaldehyde (216 g, 1.14 mol, 1.00 equiv) and 4 Å molecular sieves in dichloromethane (3 L) and THF (1 L), followed by dimethylamine (862.5 mL, 1.50 equiv). The resulting mixture was stirred for 30 min at rt and NaBH(OAc)₃ (292.6 g, 1.38 mol, 1.20 equiv) was added batchwise at 0° C. The reaction mixture was stirred overnight at rt and then quenched with water (1 L). The organic phase was separated and washed with H₂O (2×2 L). The aqueous phase was then extracted with DCM (2×1 L). The combined organic phases were dried over anhydrous sodium sulfate, filtered, and concentrated under vacuum. The residue was purified via silica gel column chromatography eluting with dichloromethane/ethyl acetate (2:1) to afford F1-1 (214.7 g, 86%) as a light yellow oil. Analytical method 5; t_{R}=0.60 min; [M+H]⁺=220.1.

### Step 2. 4-[2-[(Dimethylamino)methyl]-1-methyl-1H-imidazol-5-yl]phenol (F1-2)

Into a 3-L 4-necked round-bottom flask, purged and maintained under an inert atmosphere of nitrogen, were placed F1-1 (53.675 g, 246.11 mmol, 1.00 equiv), (4-hydroxyphenyl)boronic acid (66.55 g, 482.49 mmol, 1.50 equiv), Pd(dppf)Cl₂ (11.75 g, 16.06 mmol, 0.05 equiv), and potassium acetate (189.05 g, 1.93 mol, 6.00 equiv) in N,N-dimethylformamide (1.3 L). The resulting solution was stirred for 18 h at 90° C. in an oil bath. The reaction was repeated three times on the same scale. The batches were combined and the mixture was cooled to rt and then poured into 3.5 L of water/ice. The resulting solution was extracted with EtOAc (3×1.5 L) and the organic layers were combined. The mixture was diluted with water (1 L) and the pH value of the solution was adjusted to 4-5 with 2 M aq. HCl. The aqueous phase was extracted with EtOAc (2×1 L) and the aqueous layers were combined. The pH value of the solution was then adjusted to 11 with NH₃.H₂O. The resulting solution was extracted with DCM (6×1 L) and the organic layers were combined and concentrated under vacuum. The crude product was purified via silica gel column chromatography eluting with dichloromethane/methanol (8:1) to afford F1-2 (120 g, 53%) as a purple oil. Analytical method 5; t_{R}=0.55 min; [M+H]⁺=232.1.

### Step 3. 4-Chloro-2-(4-[2-[(dimethylamino)methyl]-1-methyl-1H-imidazol-5-yl]phenoxy)benzaldehyde (F1)

Into a 3-L 4-necked round-bottom flask were placed F1-2 (120 g, 518.82 mmol, 1.00 equiv) and potassium carbonate (214.9 g, 1.55 mol, 3.00 equiv) in N,N-dimethylformamide (2 L). The resulting mixture was stirred for 30 min at rt and 4-chloro-2-fluorobenzaldehyde (98.5 g, 621.23 mmol, 1.20 equiv). The reaction mixture was stirred for 4 h at 90° C. in an oil bath and then cooled to rt and diluted with water (3 L). The resulting solution was extracted with EtOAc (3×2 L) and the organic layers were combined. The mixture was diluted with water (1 L) and the pH value of the solution was adjusted to 2 with 2 M aq. HCl. The aqueous phase was extracted with EtOAc (3×2 L) and the aqueous layers were combined. The pH value of the solution was adjusted to 11 with NH₃.H₂O and then extracted with DCM (2×2 L). The combined organic phases were concentrated under vacuum and then purified via silica gel column chromatography eluting with dichloromethane/ethyl acetate (7:3) to afford F1 (43.8 g, 23%). ¹H NMR: (300 MHz, CDCl₃, ppm): δ 10.47 (s, 1H), 7.90 (d, J=8.4 Hz, 1H), 7.44 (d, J=8.6 Hz, 1H), 7.25-7.10 (m, 3H), 7.02 (s, 1H), 6.94 (d, J=1.9 Hz, 1H), 3.71 (s, 3H), 3.60 (s, 2H). Analytical method 5; t_{R}=1.00 min; [M+H]⁺=370.2.

The aldehydes shown in Table 25 below were prepared according to the procedure used for F1 (Example 11.1), F63 (Example 11.2), F2 (Example 11.3), F27 (Example 11.4), F34 (Example 11.5), F71 (Example 11.6), or F68 (Example 11.7).

TABLE 25
Building block F - Aldehydes

| | | |
|---|---|---|
| BB | | Synthesis procedure/LCMS |
| No. | Structure/Chemical name | |

| | | |
|---|---|---|
| F3 | 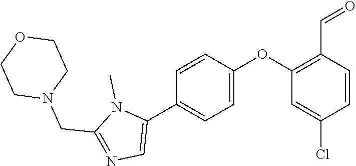 [SMILES: Cn1c(-c2ccc(Oc3cc(Cl)ccc3C=O)cc2)cnc1CN1CCOCC1] 4-chloro-2-(4-(1-methyl-2- (morpholinomethyl)-1H-imidazol-5- yl)phenoxy)benzaldehyde | F2 (Example 11.3)-starting from morpholine, 5-bromo-1-methyl-1H- imidazole-2-carbaldehyde, (4- hydroxyphenyl)boronic acid and 4- chloro-2-fluorobenzaldehyde/Analytical method 10 t_{R} = 0.84 min [M + H]⁺ = 412.1 |
| F4 | 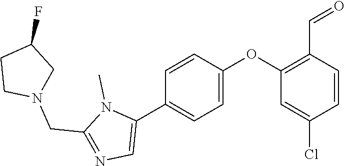 [SMILES: Cn1c(-c2ccc(Oc3cc(Cl)ccc3C=O)cc2)cnc1CN1CC[C@@H](F)C1] (R)-4-chloro-2-(4-(2-((3-fluoropyrrolidin-1- yl)methyl)-1-methyl-1H-imidazol-5- yl)phenoxy)benzaldehyde | F2 (Example 11.3)-starting from (R)-3- fluoropyrrolidine, 5-bromo-1-methyl-1H- imidazole-2-carbaldehyde, (4- hydroxyphenyl)boronic acid and 4- chloro-2-fluorobenzaldehyde/Analytical method 10 t_{R} = 0.86 min [M + H]⁺ = 414.2 |
| F5 | 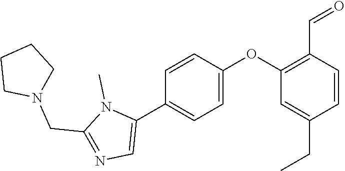 [SMILES: CCc1ccc(C=O)c(Oc2ccc(-c3cnc(CN4CCCC4)n3C)cc2)c1] 4-ethyl-2-(4-(1-methyl-2-(pyrrolidin-1- ylmethyl)-1H-imidazol-5- yl)phenoxy)benzaldehyde | F2 (Example 11.3) or F63 (Example 11.2)-starting from 5-bromo-1-methyl- 1H-imidazole-2-carbaldehyde, (4- hydroxyphenyl)boronic acid, pyrrolidine and 4-ethyl-2-fluorobenzaldehyde/ Analytical method 10 t_{R} = 0.80 min [M + H]⁺ = 390.4 |
| F6 | 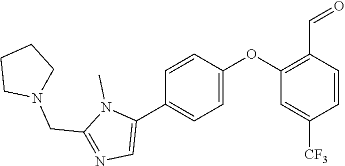 [SMILES: Cn1c(-c2ccc(Oc3cc(C(F)(F)F)ccc3C=O)cc2)cnc1CN1CCCC1] 2-(4-(1-methyl-2-(pyrrolidin-1-ylmethyl)-1H- imidazol-5-yl)phenoxy)-4- (trifluoromethyl)benzaldehyde | F2 (Example 11.3)-starting from pyrrolidine, 5-bromo-1-methyl-1H- imidazole-2-carbaldehyde, (4- iydroxyphenyl)boronic acid and 2-fluoro- 4-(trifluoromethyl)benzaldehyde/ Analytical method 10 t_{R} = 0.83 min [M + H]⁺ = 430.4 |
| F7 | 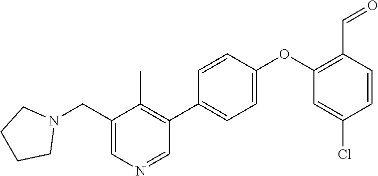 [SMILES: Cc1c(CN2CCCC2)cncc1-c1ccc(Oc2cc(Cl)ccc2C=O)cc1] 4-chloro-2-(4-(4-methyl-5-(pyrrolidin-1- ylmethyl)pyridin-3- yl)phenoxy)benzaldehyde | F2 (Example 11.3)-starting from pyrrolidine, 5-bromo-4- methylnicotinaldehyde, (4- hydroxyphenyl)boronic acid and 4- chloro-2-fluorobenzaldehyde/Analytical method 10 t_{R} = 0.82 min [M + H]⁺ = 407.3 |
| F8 | 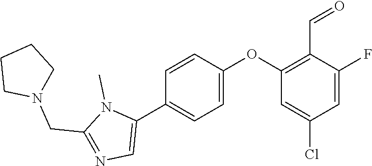 [SMILES: Cn1c(-c2ccc(Oc3cc(Cl)cc(F)c3C=O)cc2)cnc1CN1CCCC1] 4-chloro-2-(4-(4-methyl-5-(pyrrolidin-1- ylmethyl)pyridin-3- yl)phenoxy)benzaldehyde | F2 (Example 11.3)-starting from pyrrolidine, 5-bromo-1-methyl-1H- imidazole-2-carbaldehyde, (4- hydroxyphenyl)boronic acid and 4- chloro-2,6-difluorobenzaldehyde/ Analytical method 10 t_{R} = 0.81 min [M + H]⁺ = 414.2 |
| F9 | 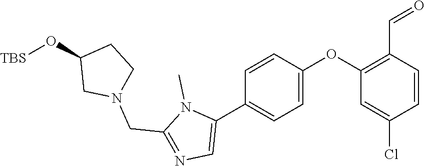 2-(4-(2-((3-((tert- butyldimethylsilyl)oxy)pyrrolidin-1- yl)methyl)-1-methyl-1H-imidazol-5- yl)phenoxy)-4-chlorobenzaldehyde | F2 (Example 11.3)-starting from (S)-3- ((tert-butyldimethylsilyl)oxy)pyrrolidine, 5-bromo-1-methyl-1H-imidazole-2- carbaldehyde, (4-hydroxyphenyl)boronic acid and 4-chloro-2-fluorobenzaldehyde as starting materials. TBS was partially lost in Step 3. Alcohol was reprotected by treatment with TBS-Cl and imidazole/ Analytical method 10 t_{R} = 1.21 min [M + H]⁺ = 526.3 |
| F10 | 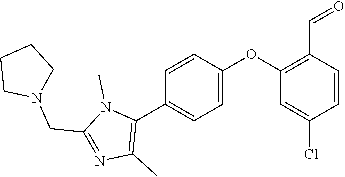 [SMILES: Cc1nc(CN2CCCC2)n(C)c1-c1ccc(Oc2cc(Cl)ccc2C=O)cc1] 4-chloro-2-(4-(1,4-dimethyl-2-(pyrrolidin-1- ylmethyl)-1H-imidazol-5- yl)phenoxy)benzaldehyde trifluoroacetate | F2 (Example 11.3)-starting from pyrrolidine, F10-1 (mixture of regioisomers) (see Example 11.9), (4- hydroxyphenyl)boronic acid and 4- chloro-2-fluorobenzaldehyde/Analytical method 10 t_{R} = 0.83 min [M + H]⁺ = 410.2 |
| F11 | 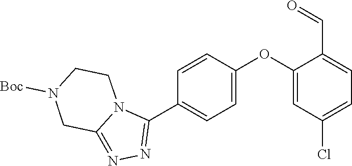 [SMILES: CC(C)(C)OC(=O)N1CCn2c(nnc2-c2ccc(Oc3cc(Cl)ccc3C=O)cc2)C1] tert-butyl 3-(4-(5-chloro-2- formylphenoxy)phenyl)-5,6-dihydro- [1,2,4]triazolo[4,3-a]pyrazine-7(8H)- carboxylate | F2 (Example 11.3)-starting from tert- butyl 3-bromo-5,6-dihydro- [1,2,4]triazolo[4,3-a]pyrazine-7(8H)- carboxylate, (4-hydroxyphenyl)boronic acid and 4-chloro-2-fluorobenzaldehyde/ Analytical method 10 t_{R} = 1.07 min [M + H]⁺ = 455.2 |
| F12 | 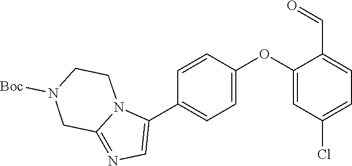 [SMILES: CC(C)(C)OC(=O)N1CCn2c(-c3ccc(Oc4cc(Cl)ccc4C=O)cc3)cnc2C1] tert-butyl 3-(4-(5-chloro-2- formylphenoxy)phenyl)-5,6- dihydroimidazo[1,2-a]pyrazine-7(8H)- carboxylate | F2 (Example 11.3)-starting from tert- butyl 3-bromo-5,6-dihydroimidazo[1,2- a]pyrazine-7(8H)-carboxylate, (4- hydroxyphenyl)boronic acid and 4- chloro-2-fluorobenzaldehyde/Analytical method 10 t_{R} = 1.04 min [M + H]⁺ = 454. |
| F13 | 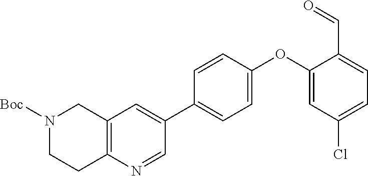 [SMILES: CC(C)(C)OC(=O)N1CCc2ncc(-c3ccc(Oc4cc(Cl)ccc4C=O)cc3)cc2C1] tert-butyl 3-(4-(5-chloro-2- formylphenoxy)phenyl)-7,8-dihydro-1,6- naphthyridine-6(5H)-carboxylate | F2 (Example 11.3)-starting from F13-1 (see Example 11.10), (4- hydroxyphenyl)boronic acid and 4- chloro-2-fluorobenzaldehyde/Analytical method 10 t_{R} = 1.36 min [M + H]⁺ = 465.3 |
| F14 | 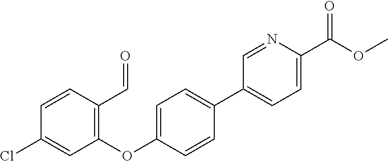 [SMILES: COC(=O)c1ccc(-c2ccc(Oc3cc(Cl)ccc3C=O)cc2)cn1] methyl 5-(4-(5-chloro-2- formylphenoxy)phenyl)picolinate | F2 (Example 11.3)-starting from methyl 5-bromopicolinate, (4- hydroxyphenyl)boronic acid and 4- chloro-2-fluorobenzaldehyde/Analytical method 10 t_{R} = 1.16 min [M + H]⁺ = 368.1 |
| F15 | 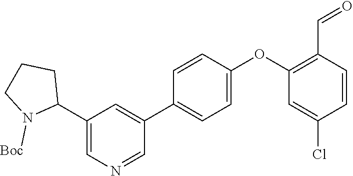 [SMILES: CC(C)(C)OC(=O)N1CCCC1c1cncc(-c2ccc(Oc3cc(Cl)ccc3C=O)cc2)c1] tert-butyl 2-(5-(4-(5-chloro-2- formylphenoxy)phenyl)pyridin-3- yl)pyrrolidine-1-carboxylate (racemate) | F2 (Example 11.3)-starting from F15-1 (see Table 26), (4- hydroxyphenyl)boronic acid and 4- chloro-2-fluorobenzaldehyde/Analytical method 10 t_{R} = 1.34 min [M + H]⁺ = 479.2 |
| F16 | 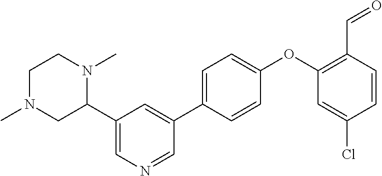 [SMILES: CN1CCN(C)C(c2cncc(-c3ccc(Oc4cc(Cl)ccc4C=O)cc3)c2)C1] 4-chloro-2-(4-(5-(1,4-dimethylpiperazin-2- yl)pyridin-3-yl)phenoxy)benzaldehyde (racemate) | F2 (Example 11.3)-starting from F16-2 (see Example 11.18), (4- hydroxyphenyl)boronic acid and 4- chloro-2-fluorobenzaldehyde/Analytical method 10 t_{R} = 0.80 min [M + H]⁺ = 422.3 |
| F17 | 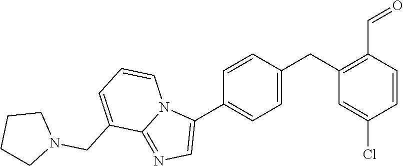 [SMILES: O=Cc1ccc(Cl)cc1Cc1ccc(-c2cnc3c(CN4CCCC4)cccn23)cc1] 4-chloro-2-(4-(8-(pyrrolidin-1- ylmethyl)imidazo[1,2-a]pyridin-3- yl)phenoxy)benzaldehyde | F2 (Example 11.3)-starting from F17-2 (see Example 11.19), (4- hydroxyphenyl)boronic acid and 4- chloro-2-fluorobenzaldehyde/Analytical method 10 t_{R} = 0.88 min [M + H]⁺ = 432.3 |
| F18 | 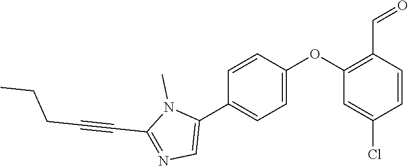 [SMILES: CCCC#Cc1ncc(-c2ccc(Oc3cc(Cl)ccc3C=O)cc2)n1C] 4-chloro-2-(4-(1-methyl-2-(pent-1-yn-1-yl)- 1H-imidazol-5-yl)phenoxy)benzaldehyde | F2 (Example 11.3)-starting from F18-4 (see Example 11.21) and 4-chloro-2- fluorobenzaldehyde/Analytical method 10 t_{R} = 1.23 min [M + H]⁺ = 379.2 |
| F19 | 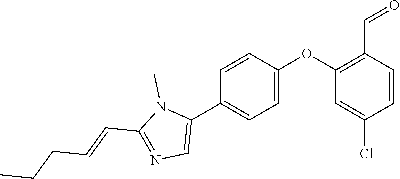 [SMILES: CCC/C=C/c1ncc(-c2ccc(Oc3cc(Cl)ccc3C=O)cc2)n1C] (E)-4-chloro-2-(4-(1-methyl-2-(pent-1-en-1- yl)-1H-imidazol-5-yl)phenoxy)benzaldehyde | F2 (Example 11.3)-starting from F19-2 (see Example 11.22) and 4-chloro-2- fluorobenzaldehyde/Analytical method 10 t_{R} = 0.95 min [M + H]⁺ = 381.2 |
| F20 | 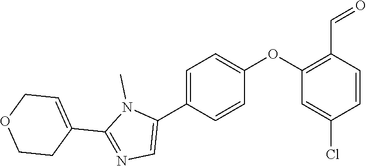 [SMILES: Cn1c(-c2ccc(Oc3cc(Cl)ccc3C=O)cc2)cnc1C1=CCOCC1] 4-chloro-2-(4-(2-(3,6-dihydro-2H-pyran-4- yl)-1-methyl-1H-imidazol-5- yl)phenoxy)benzaldehyde | F2 (Example 11.3)-starting from F20-2 (see Example 11.23) and 4-chloro-2- fluorobenzaldehyde/Analytical method 10 t_{R} = 0.81 min [M + H]⁺ = 395.1 |
| F21 | 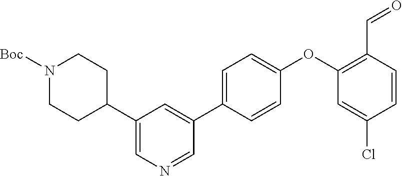 [SMILES: CC(C)(C)OC(=O)N1CCC(c2cncc(-c3ccc(Oc4cc(Cl)ccc4C=O)cc3)c2)CC1] tert-butyl 4-(5-(4-(5-chloro-2- formylphenoxy)phenyl)pyridin-3- yl)piperidine-1-carboxylate | F2 (Example 11.3)-starting from F21-3 (see Example 11.24) and 4-chloro-2- fluorobenzaldehyde/Analytical method 10 t_{R} = 1.39 min [M + H]⁺ = 493.4 |
| F22 | 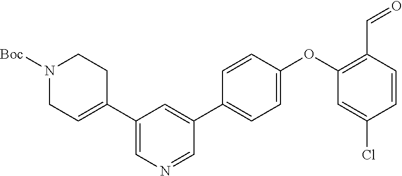 [SMILES: CC(C)(C)OC(=O)N1CC=C(c2cncc(-c3ccc(Oc4cc(Cl)ccc4C=O)cc3)c2)CC1] tert-butyl 5-(4-(5-chloro-2- formylphenoxy)phenyl)-3′,6′-dihydro-[3,4′- bipyridine]⁻1′(2′H)-carboxylate | F2 (Example 11.3)-starting from F21-2 (see Example 11.24) and 4-chloro-2- fluorobenzaldehyde/Analytical method 10 t_{R} = 1.44 min [M + H]⁺ = 491.3 |
| F23 | 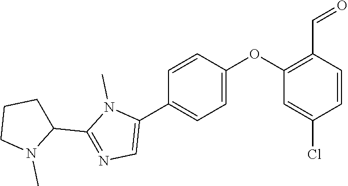 [SMILES: CN1CCCC1c1ncc(-c2ccc(Oc3cc(Cl)ccc3C=O)cc2)n1C] 4-chloro-2-(4-(1-methyl-2-(1- methylpyrrolidin-2-yl)-1H-imidazol-5- yl)phenoxy)benzaldehyde (racemate) | F2 (Example 11.3)-starting from F23-2 (see Example 11.25) and 4-chloro-2- fluorobenzaldehyde/Analytical method 10 t_{R} = 0.81 min [M + H]⁺ = 396.3 |
| F24 | 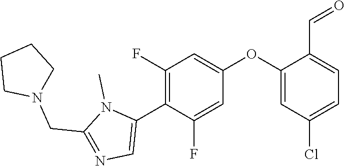 [SMILES: Cn1c(-c2c(F)cc(Oc3cc(Cl)ccc3C=O)cc2F)cnc1CN1CCCC1] 4-chloro-2-(3,5-difluoro-4-(1-methyl-2- (pyrrolidin-1-ylmethyl)-1H-imidazol-5- yl)phenoxy)benzaldehyde | F2 (Example 11.3)-starting from F24-3 (see Example 11.26) and 4-chloro-2- fluorobenzaldehyde/Analytical method 10 t_{R} = 0.82 min [M + H]⁺ = 432.1 |
| F25 | 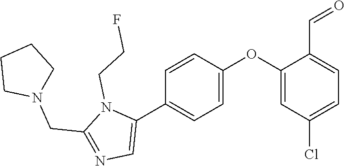 [SMILES: O=Cc1ccc(Cl)cc1Oc1ccc(-c2cnc(CN3CCCC3)n2CCF)cc1] 4-choro-2-(4-(1-(2-fluoroethyl)-2- (pyrrolidin-1-ylmethyl)-1H-imidazol-5- yl)phenoxy)benzaldehyde | F2 (Example 11.3) starting from F25-3 (see Example 11.27) and 4-chloro-2- fluorobenzaldehyde/Analytical method 10 t_{R} = 0.84 min [M + H]⁺ = 428.2 |
| F26 | 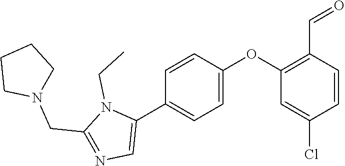 [SMILES: CCn1c(-c2ccc(Oc3cc(Cl)ccc3C=O)cc2)cnc1CN1CCCC1] 4-chloro-2-(4-(1-ethyl-2-(pyrrolidin-1- ylmethyl)-1H-imidazol-5- yl)phenoxy)benzaldehyde | F2 (Example 11.3)-starting from F26-2 (see Example 11.27) and 4-chloro-2- fluorobenzaldehyde/Analytical method 10 t_{R} = 0.88 min [M + H]⁺ = 410.1 |
| F28 | 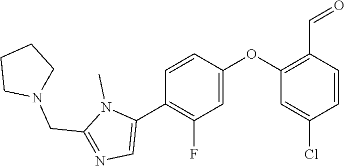 [SMILES: Cn1c(-c2ccc(Oc3cc(Cl)ccc3C=O)cc2F)cnc1CN1CCCC1] 4-chloro-2-(3-fluoro-4-(1-methyl-2- (pyrrolidin-1-ylmethyl)-1H-imidazol-5- yl)phenoxy)benzaldehyde | F27 (Example 11.4)-starting from F2-1 (see Example 11.3), (4-(benzyloxy)-2- fluorophenyl)boronic acid and 4-chloro-2- fluorobenzaldehyde/ Analytical method 10 t_{R} = 0.82 min [M + H]⁺ = 414.3 |
| F29 | 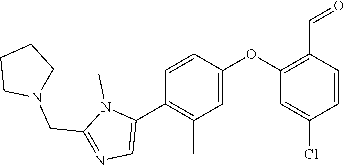 [SMILES: Cc1cc(Oc2cc(Cl)ccc2C=O)ccc1-c1cnc(CN2CCCC2)n1C] 4-chloro-2-(3-methyl-4-(1-methyl-2- (pyrrolidin-1-ylmethyl)-1H-imidazol-5- yl)phenoxy)benzaldehyde | F27 (Example 11.4)-starting from F2-1 (see Example 11.3), (4-(benzyloxy)-2- methylphenyl)boronic acid and 4-chloro- 2-fluorobenzaldehyde/Analytical method 10 t_{R} = 0.85 min [M + H]⁺ = 410.3 |
| F30 | 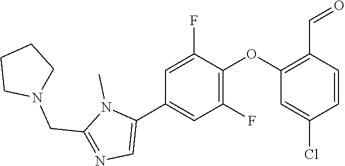 [SMILES: Cn1c(-c2cc(F)c(Oc3cc(Cl)ccc3C=O)c(F)c2)cnc1CN1CCCC1] 4-chloro-2-(2,6-difluoro-4-(1-methyl-2- (pyrrolidin-1-ylmethyl)-1H-imidazol-5- yl)phenoxy)benzaldehyde | F27 (Example 11.4)-starting from F2-1 (see Example 11.3), (4-(benzyloxy)-3,5- difluorophenyl)boronic acid and 4-chloro- 2-fluorobenzaldehyde/Analytical method 10 t_{R} = 0.81 min [M + H]⁺ = 432.2 |
| F31 | 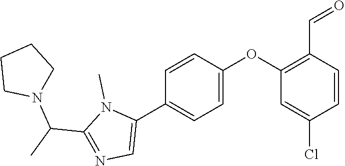 [SMILES: CC(c1ncc(-c2ccc(Oc3cc(Cl)ccc3C=O)cc2)n1C)N1CCCC1] 4-chloro-2-(4-(1-methyl-2-(1-(pyrrolidin-1- yl)ethyl)-1H-imidazol-5- yl)phenoxy)benzaldehyde (racemate) | F27 (Example 11.4)-starting from F31-2 (see Example 11.20), (4- (benzyloxy)phenyl)boronic acid and 4- chloro-2-fluorobenzaldehyde/Analytical method 10 t_{R} = 0.83 min [M + H]⁺ = 410.0 |
| F32 | 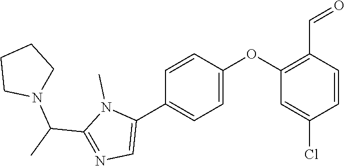 [SMILES: CC(c1ncc(-c2ccc(Oc3cc(Cl)ccc3C=O)cc2)n1C)N1CCCC1] 4-chloro-2-(4-(1-methyl-2-(1-(pyrrolidin-1- yl)ethyl)-1H-imidazol-5- yl)phenoxy)benzaldehyde (enantiomer of F33) | F27 (Example 11.4)-obtained after separation of the racemate F31 by chiral chromatography/Analytical method 10 t_{R} = 0.84 min [M + H]⁺ = 410.3 |
| F33 | 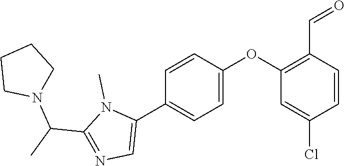 [SMILES: CC(c1ncc(-c2ccc(Oc3cc(Cl)ccc3C=O)cc2)n1C)N1CCCC1] 4-chloro-2-(4-(1-methyl-2-(1-(pyrrolidin-1- yl)ethyl)-1H-imidazol-5- yl)phenoxy)benzaldehyde (enantiomer of F32) | F27 (Example 11.4)-obtained after separation of the racemate F31 by chiral chromatography/Analytical method 10 t_{R} = 0.85 min [M + H]⁺ = 410.2 |
| F35 | 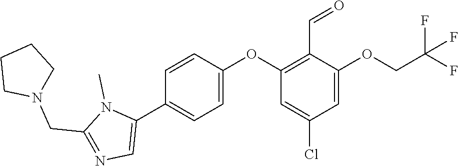 [SMILES: Cn1c(-c2ccc(Oc3cc(Cl)cc(OCC(F)(F)F)c3C=O)cc2)cnc1CN1CCCC1] 4-chloro-2-(4-(1-methyl-2-(pyrrolidin-1- ylmethyl)-1H-imidazol-5-yl)phenoxy)-6- (2,2,2-trifluoroethoxy)-benzaldehyde | F34 (Example 11.5)-starting from F2-2 (see Example 11.3)/ Analytical method 10 t_{R} = 0.87 min [M + H]⁺ = 494.2 |
| F36 | 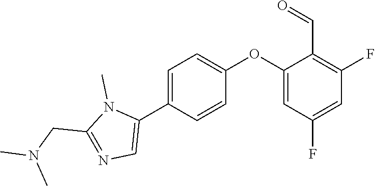 [SMILES: CN(C)Cc1ncc(-c2ccc(Oc3cc(F)cc(F)c3C=O)cc2)n1C] 2-(4-(2-((dimethylamino)methyl)-1-methyl- 1H-imidazol-5-yl)phenoxy)-4,6- difluorobenzaldehyde | F1 (Example 11.1)-starting from F1-2 and the respective substituted aldehyde/ Analytical method 7 t_{R} = 0.69 min [M + H]⁺ = 372.2 |
| F37 | 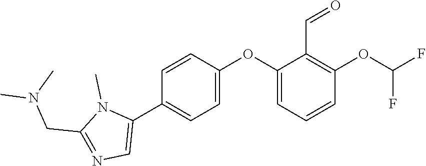 [SMILES: CN(C)Cc1ncc(-c2ccc(Oc3cccc(OC(F)F)c3C=O)cc2)n1C] 2-(difluoromethoxy)-6-(4-(2- ((dimethylamino)methyl)-1-methyl-1H- imidazol-5-yl)phenoxy) benzaldehyde | F1 (Example 11.1)-starting from F1-2 and the respective substituted aldehyde/ Analytical method 7 t_{R} = 0.66 min [M + H]⁺ = 402.1 |
| F38 | 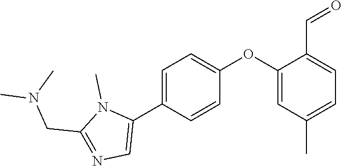 [SMILES: Cc1ccc(C=O)c(Oc2ccc(-c3cnc(CN(C)C)n3C)cc2)c1] 2-(4-(2-((dimethylamino)methyl)-1-methyl- 1H-imidazol-5-yl)phenoxy)-4- methylbenzaldehyde | F1 (Example 11.1)-starting from F1-2 and the respective substituted aldehyde/ Analytical method 7 t_{R} = 0.67 min [M + H]⁺ = 350.3 |
| F39 | 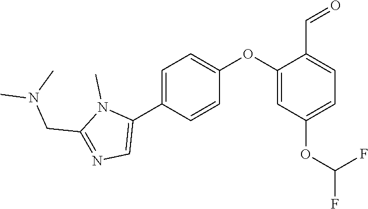 [SMILES: CN(C)Cc1ncc(-c2ccc(Oc3cc(OC(F)F)ccc3C=O)cc2)n1C] 4-(difluoromethoxy)-2-(4-(2- ((dimethylamino)methyl)-1-methyl-1H- imidazol-5-yl)phenoxy)benzaldehyde | F1 (Example 11.1)-starting from F1-2 and the respective substituted aldehyde/ Analytical method 7 t_{R} = 0.59 min [M + H]⁺ = 402.2 |
| F40 | 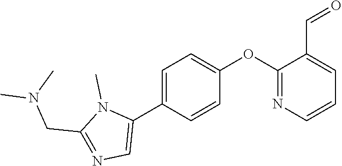 [SMILES: CN(C)Cc1ncc(-c2ccc(Oc3ncccc3C=O)cc2)n1C] 2-(4-(2-((dimethylamino)methyl)-1-methyl- 1H-imidazol-5-yl)phenoxy)nicotinaldehyde | F1 (Example 11.1)-starting from F1-2 and the respective substituted aldehyde/ Analytical method 7 t_{R} = 0.50 min [M + H]⁺ = 336.7 |
| F41 | 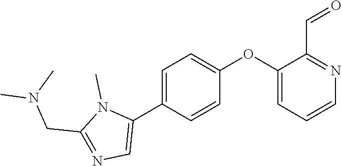 [SMILES: CN(C)Cc1ncc(-c2ccc(Oc3cccnc3C=O)cc2)n1C] 3-(4-(2-((dimethylamino)methyl)-1-methyl- 1H-imidazol-5-yl)phenoxy)picolinaldehyde | F1 (Example 11.1)-starting from F1-2 and the respective substituted aldehyde/ Analytical method 1 t_{R} = 0.59 min [M + H]⁺ = 337.2 |
| F42 | 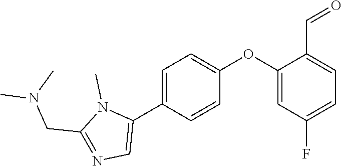 [SMILES: CN(C)Cc1ncc(-c2ccc(Oc3cc(F)ccc3C=O)cc2)n1C] 2-(4-(2-((dimethylamino)methyl)-1-methyl- 1H-imidazol-5-yl)phenoxy)-4- fluorobenzaldehyde | F1 (Example 11.1)-starting from F1-2 and the respective substituted aldehyde/ Analytical method 7 t_{R} = 0.61 min [M + H]⁺ = 354.2 |
| F43 | 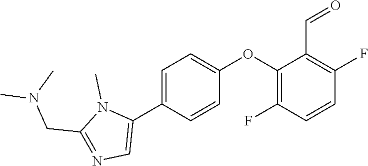 [SMILES: CN(C)Cc1ncc(-c2ccc(Oc3c(F)ccc(F)c3C=O)cc2)n1C] 2-(4-(2-((dimethylamino)methyl)-1-methyl- 1H-imidazol-5-yl)phenoxy)-3,6- difluorobenzaldehyde | F1 (Example 11.1)-starting from F1-2 and the respective substituted aldehyde/ Analytical method 7 t_{R} = 0.89 min [M + H]⁺ = 372.4 |
| F44 | 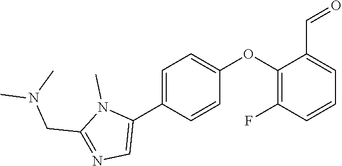 [SMILES: CN(C)Cc1ncc(-c2ccc(Oc3c(F)cccc3C=O)cc2)n1C] 2-(4-(2-((dimethylamino)methyl)-1-methyl- 1H-imidazol-5-yl)phenoxy)-3- fluorobenzaldehyde | F1 (Example 11.1)-starting from F1-2 and the respective substituted aldehyde/ Analytical method 7 t_{R} = 0.91 min [M + H]⁺ = 354.3 |
| F45 | 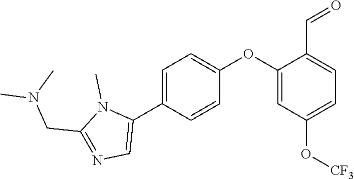 [SMILES: CN(C)Cc1ncc(-c2ccc(Oc3cc(OC(F)(F)F)ccc3C=O)cc2)n1C] 2-(4-(2-((dimethylamino)methyl)-1-methyl- 1H-imidazol-5-yl)phenoxy)-4- (trifluoromethoxy)benzaldehyde | F1 (Example 11.1)-starting from F1-2 and the respective substituted aldehyde/ Analytical method 7 t_{R} = 0.70 min [M + H]⁺ = 420.4 |
| F46 | 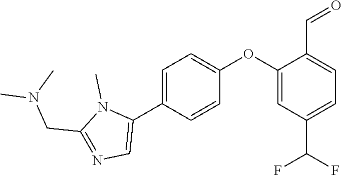 [SMILES: CN(C)Cc1ncc(-c2ccc(Oc3cc(C(F)F)ccc3C=O)cc2)n1C] 4-(difluoromethyl)-2-(4-(2- ((dimethylamino)methyl)-1-methyl-1H- imidazol-5-yl)phenoxy)benzaldehyde | F1 (Example 11.1)-starting from F1-2 and F46-1 (see Example 11.34)/ Analytical method 5 t_{R} = 0.99 min [M + H]⁺ = 386.2 |
| F47 | 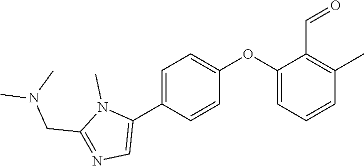 2-(4-(2-((dimethylamino)methyl)-1-methyl- 1H-imidazol-5-yl)phenoxy)-6- methylbenzaldehyde | F1 (Example 11.1)-starting from F1-2 and the respective substituted aldehyde/ Analytical method 5 t_{R} = 0.98 min [M + H]⁺ = 350.1 |
| F48 | 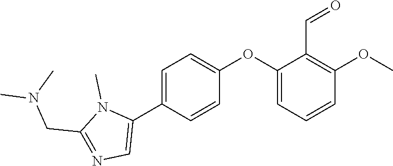 2-(4-(2-((dimethylamino)methyl)-1-methyl- 1H-imidazol-5-yl)phenoxy)-6- methoxybenzaldehyde | F1 (Example 11.1)-starting from F1-2 and the respective substituted aldehyde/ Analytical method 5 t_{R} = 0.85 min [M + H]⁺ = 366.2 |
| F49 | 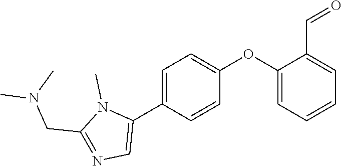 [SMILES: CN(C)Cc1ncc(-c2ccc(Oc3ccccc3C=O)cc2)n1C] 2-(4-(2-((dimethylamino)methyl)-1-methyl- 1H-imidazol-5-yl)phenoxy)benzaldehyde | F1 (Example 11.1-starting from F1-2 and the respective substituted aldehyde/ Analytical method 7 t_{R} = 1.00 min [M + H]⁺ = 336.2 |
| F50 | 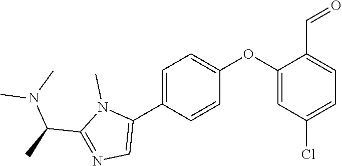 [SMILES: C[C@H](c1ncc(-c2ccc(Oc3cc(Cl)ccc3C=O)cc2)n1C)N(C)C] (R)-4-chloro-2-(4-(2-(1- (dimethylamino)ethyl)-1-methyl-1H- imidazol-5-yl)phenoxy) benzaldehyde | F1 (Example 11.1)-starting from F50-4 (see Example 11.13), (4- hydroxyphenyl)boronic acid and 4- chloro-2-fluorobenzaldehyde/ Analytical method 5 t_{R} = 1.03 min [M + H]⁺ = 384.3 |
| F51 | 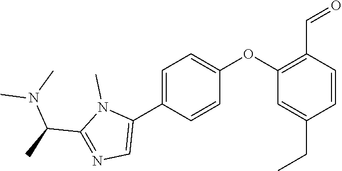 [SMILES: CCc1ccc(C=O)c(Oc2ccc(-c3cnc([C@@H](C)N(C)C)n3C)cc2)c1] (R)-2-(4-(2-(1-(dimethylamino)ethyl)-1- methyl-1H-imidazol-5-yl)phenoxy)-4- ethylbenzaldehyde | F1 (Example 11.1)-starting from F50-4 (see Example 11.13), (4- hydroxyphenyl)boronic acid and 4-ethyl- 2-fluorobenzaldehyde/ Analytical method 5 t_{R} = 1.08 min [M + H]⁺ = 378.1 |
| F52 | 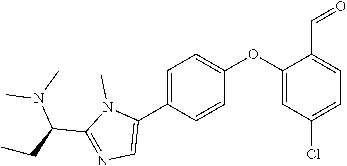 [SMILES: CC[C@H](c1ncc(-c2ccc(Oc3cc(Cl)ccc3C=O)cc2)n1C)N(C)C] (R)-4-chloro-2-(4-(2-(1- (dimethylamino)propyl)-1-methyl-1H- imidazol-5-yl)phenoxy)benzaldehyde | F1 (Example 11.1)-F1 starting from F52-1 (see Error! Reference source not found.), (4-hydroxyphenyl)boronic acid and 4-chloro-2-fluorobenzaldehyde/ Analytical method 5 t_{R} = 1.10 min [M + H]⁺ = 398.1 |
| F53 | 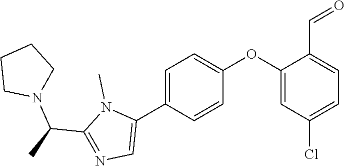 [SMILES: C[C@H](c1ncc(-c2ccc(Oc3cc(Cl)ccc3C=O)cc2)n1C)N1CCCC1] (R)-4-chloro-2-(4-(1-methyl-2-(1-(pyrrolidin- 1-yl)ethyl)-1H-imidazol-5- yl)phenoxy)benzaldehyde | F1 (Example 11.1)-starting from F53-1 (see Example 11.14), (4- hydroxyphenyl)boronic acid and 4- chloro-2-fluorobenzaldehyde/ Analytical method 5 t_{R} = 1.13 min [M + H]⁺ = 410 2. |
| F54 | 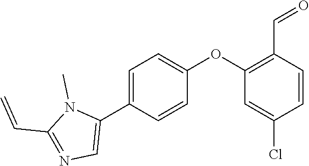 [SMILES: C=Cc1ncc(-c2ccc(Oc3cc(Cl)ccc3C=O)cc2)n1C] 4-chloro-2-(4-(1-methyl-2-vinyl-1H- imidazol-5-yl)phenoxy)benzaldehyde | F1 (Example 11.1)-starting from F54-1 (see Example 11.15), (4- hydroxyphenyl)boronic acid and 4- chloro-2-fluorobenzaldehyde/ Analytical method 5 t_{R} = 1.17 min [M + H]⁺ = 339.1 |
| F55 | 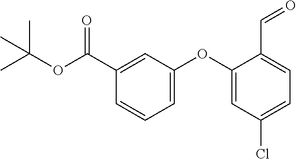 [SMILES: CC(C)(C)OC(=O)c1cccc(Oc2cc(Cl)ccc2C=O)c1] tert-butyl 3-(5-chloro-2- formylphenoxy)benzoate | F1 (Example 11.1)-starting from tert- butyl 3-hydroxybenzoate and 4-chloro-2- fluorobenzaldehyde/ Analytical method 7 t_{R} = 1.50 min [M-tBu + H]⁺ = 277.2 |
| F56 | 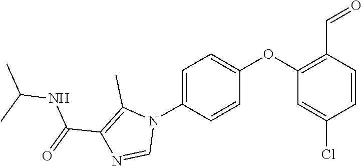 [SMILES: Cc1c(C(=O)NC(C)C)ncn1-c1ccc(Oc2cc(Cl)ccc2C=O)cc1] -4-(5-chloro-2-formylphenoxy)phenyl)-N- isopropyl-1H-imidazole-4-carboxamide | F27 (Example 11.4)-starting from F56-2 (Example 11.29) and 4-chloro-2- fluorobenzaldehyde/Analytical method 4 t_{R} = 2.37 min [M + H]⁺ = 398.1 |
| F57-2 | 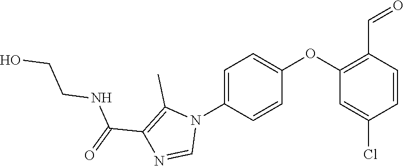 [SMILES: Cc1c(C(=O)NCCO)ncn1-c1ccc(Oc2cc(Cl)ccc2C=O)cc1] 1-(4-(5-chloro-2-formylphenoxy)phenyl)-N- (2-hydroxyethyl)-1H-imidazole-4- carboxamide | F27 (Example 11.4)-starting from F57-1 (see Table 31) and 4-chloro-2- fluorobenzaldehyde/Analytical method 7 t_{R} = 0.96 min [M + H]⁺ = 400.1 |
| F58 | 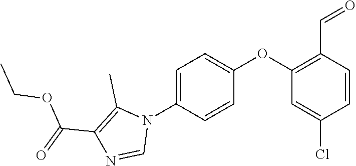 [SMILES: CCOC(=O)c1ncn(-c2ccc(Oc3cc(Cl)ccc3C=O)cc2)c1C] ethyl 1-(4-(5-chloro-2- formylphenoxy)phenyl)-5-methyl-1H- imidazole-4-carboxylate | F27 (Example 11.4)-starting from F59-3 (see Example 11.28) and 4-chloro-2- fluorobenzaldehyde/Analytical method 1 t_{R} = 1.28 min [M + H]⁺ = 385.3 |
| F59 | 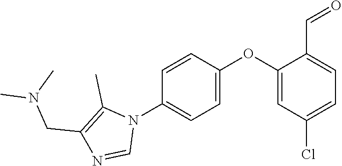 [SMILES: Cc1c(CN(C)C)ncn1-c1ccc(Oc2cc(Cl)ccc2C=O)cc1] 4-chloro-2-(4-(4-((dimethylamino)methyl)-5- methyl-1H-imidazol-1- yl)phenoxy)benzaldehyde | F27 (Example 11.4)-starting from F59-7 (see Example 11.28 and 4-chloro-2- fluorobenzaldehyde/Analytical method 5 t_{R} = 1.02 min [M + H]⁺ = 370.2 |
| F60 | 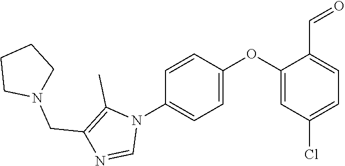 [SMILES: Cc1c(CN2CCCC2)ncn1-c1ccc(Oc2cc(Cl)ccc2C=O)cc1] 4-chloro-2-(4-(5-methyl-4-(pyrrolidin-1- ylmethyl)-1H-imidazol-1- yl)phenoxy)benzaldehyde | F27 (Example 11.4)-starting from F60-1 (see Error! Reference source not found.) and 4-chloro-2-fluorobenzaldehyde/ Analytical method 7 t_{R} = 0.78 min [M + H]⁺ = 396.2 |
| F61 | 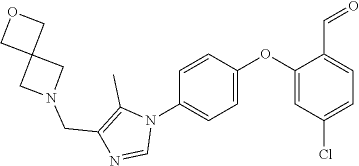 [SMILES: Cc1c(CN2CC3(COC3)C2)ncn1-c1ccc(Oc2cc(Cl)ccc2C=O)cc1] 2-(4-(4-((2-oxa-6-azaspiro[3.3]heptan-6- yl)methyl)-5-methyl-1H-imidazol-1- yl)phenoxy)-4-chlorobenzaldehyde | F27 (Example 11.4)-starting from F61-1 (see Error! Reference source not found.) and 4-chloro-2-fluorobenzaldehyde/ Analytical method 5 t_{R} = 0.90 min [M + H]⁺ = 424.0 |
| F62 | 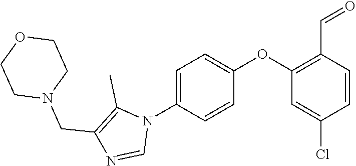 [SMILES: Cc1c(CN2CCOCC2)ncn1-c1ccc(Oc2cc(Cl)ccc2C=O)cc1] 4-chloro-2-(4-(5-methyl-4- (morpholinomethyl)-1H-imidazol-1- yl)phenoxy)benzaldehyde | F27 (Example 11.4)-starting from F62-5 (see Example 1130) and 4-chloro-2- fluorobenzaldehyde Analytical method 5 t_{R} = 0.94 min [M + H]⁺ = 412.2 |
| F64 | 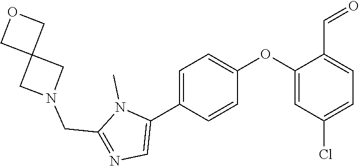 [SMILES: Cn1c(-c2ccc(Oc3cc(Cl)ccc3C=O)cc2)cnc1CN1CC2(COC2)C1] 2-(4-(2-((2-oxa-6-azaspiro[3.3]heptan-6- yl)methyl)-1-methyl-1H-imidazol-5- yl)phenoxy)-4-chlorobenzaldehyde | F63 (Example 11.2)-starting from 5- bromo-1-methyl-1H-imidazole-2- carbaldehyde, 4-(4,4,5,5-tetramethyl- 1,3,2-dioxaborolan-2-yl)phenol, 2-oxa-6- azaspiro[3.3]heptane and 4-chloro-2- fluorobenzaldehyde/ Analytical method 5 t_{R} = 0.91 min [M + H]⁺ = 424.2 |
| F65 | 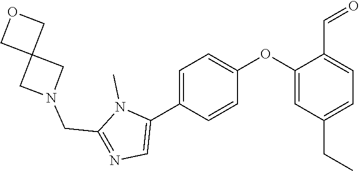 [SMILES: CCc1ccc(C=O)c(Oc2ccc(-c3cnc(CN4CC5(COC5)C4)n3C)cc2)c1] 2-(4-(2-((2-oxa-6-azaspiro[3.3]heptan-6- yl)methyl)-1-methyl-1H-imidazol-5- yl)phenoxy)-4-ethylbenzaldehyde | F63 (Example 11.2)-starting from 5- bromo-1-methyl-1H-imidazole-2- carbaldehyde, (4-hydroxyphenyl)boronic acid, 2-oxa-6-azaspiro[3.3]heptane and 4-ethyl-2-fluorobenzaldehyde/ Analytical method 7 t_{R} = 0.79 min [M + H]⁺ = 418.3 |
| F66 | 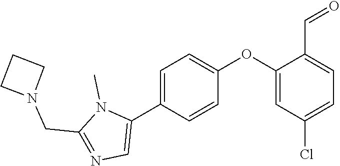 [SMILES: Cn1c(-c2ccc(Oc3cc(Cl)ccc3C=O)cc2)cnc1CN1CCC1] 2-(4-(2-(azetidin-1-ylmethyl)-1-methyl-1H- imidazol-5-yl)phenoxy)-4- chlorobenzaldehyde | F63 (Example 11.2)-5-bromo-1-methyl- 1H-imidazole-2-carbaldehyde, 4-(4,4,5,5- tetramethyl-1,3,2-dioxaborolan-2- yl)phenol, azetidine and 4-chloro-2- fluorobenzaldehyde/ Analytical method 5 t_{R} = 0.99 min [M + H]⁺ = 382.2 |
| F69 | 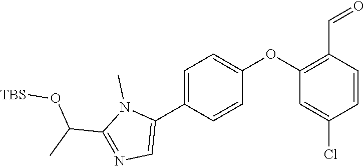 2-(4-(2-(1-((tert- butyldimethylsilyl)oxy)ethyl)-1-methyl-1H- imidazol-5-yl)phenoxy)-4- chlorobenzaldehyde (racemate) | F68 (Example 11.7)-starting from F69-1 (see Table 28)/ Analytical method 5 t_{R} = 1.61 min [M + H]⁺ = 471.1 |
| F70 | 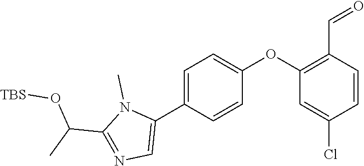 2-(4-(2-(1-((tert- butyldimethylsilyl)oxy)ethyl)-1-methyl-1H- imidazol-5-yl)phenoxy)-4- chlorobenzaldehyde (single enantiomer) | F68 (Example 11.7)-starting from F70-1 (see Example 11.16) Analytical method 5 t_{R} = 1.61 min [M + H]⁺ = 471.1 |
| F72 | 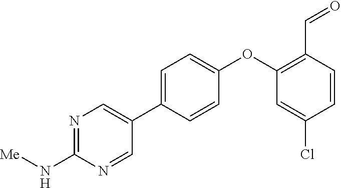 [SMILES: CNc1ncc(-c2ccc(Oc3cc(Cl)ccc3C=O)cc2)cn1] 4-chloro-2-(4-(2-(methylamino)pyrimidin-5- yl)phenoxy)benzaldehyde | F71 (Example 11.6)-starting from F71-1 and 5-bromo-N-methylpyrimidin-2-amine/ Analytical method 8 t_{R} = 1.38 min [M + H]⁺ = 339.0 |
| F73 | 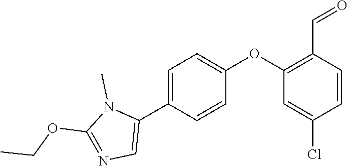 [SMILES: CCOc1ncc(-c2ccc(Oc3cc(Cl)ccc3C=O)cc2)n1C] 4-chloro-2-(4-(2-ethoxy-1-methyl-1H- imidazol-5-yl)phenoxy)benzaldehyde | F71 (Example 11.6)-starting from F71-1 and F73-1 (see Example 11.17) Analytical method 1 t_{R} = 1.09 min [M + H]⁺ = 357.3 |
| F74 | 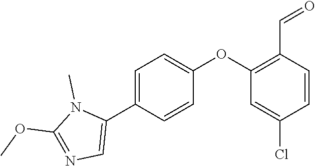 [SMILES: COc1ncc(-c2ccc(Oc3cc(Cl)ccc3C=O)cc2)n1C] 4-chloro-2-(4-(2-methoxy-1-methyl-1H- imidazol-5-yl)phenoxy)benzaldehyde | F71 (Example 11.6)-starting from F71-1 and F74-1 (see Table 29)/ Analytical method 1 t_{R} = 1.07 min [M + H]⁺ = 343.0 |
| F75 | 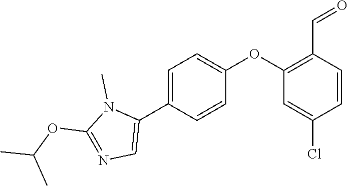 [SMILES: CC(C)Oc1ncc(-c2ccc(Oc3cc(Cl)ccc3C=O)cc2)n1C] 4-chloro-2-(4-(2-isopropoxy-1-methyl-1H- imidazol-5-yl)phenoxy)benzaldehyde | F71 (Example 11.6)-starting from F71-1 and F75-1 (see Table 29) Analytical method 1 t_{R} = 1.15 min [M + H]⁺ = 371.0 |
| F80 | 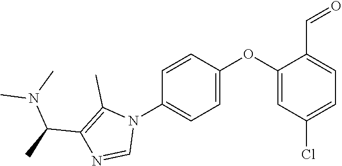 [SMILES: Cc1c([C@@H](C)N(C)C)ncn1-c1ccc(Oc2cc(Cl)ccc2C=O)cc1] (R)-4-chloro-2-(4-(4-(1- (dimethylamino)ethyl)-5-methyl-1H- imidazol-1-yl)phenoxy)benzaldehyde | F63 (Example 11.2)-F80-2 (see Table 27) and 4-chloro-2-fluorobenzaldehyde/ Analytical method 5 t_{R} = 1.08 min [M + H]⁺ = 384.0 |

### Example 11.2: Synthesis of 2-(4-(2-((bis(methyl-d3)amino)methyl)-1-methyl-1H-imidazol-5-yl)phenoxy)-4-chlorobenz-aldehyde (F63)

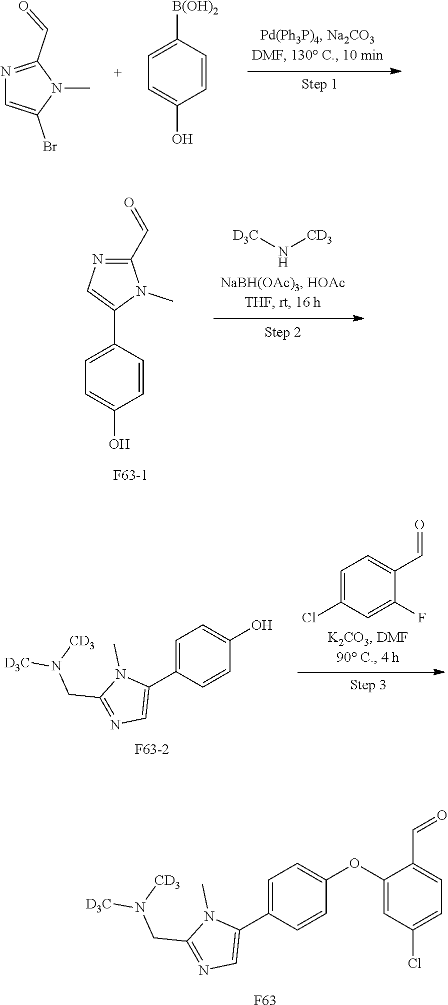

### Step 1. 5-(4-Hydroxyphenyl)-1-methyl-1H-imidazole-2-carbaldehyde (F63-1)

To a solution of 5-bromo-1-methyl-1H-imidazole-2-carbaldehyde (500 mg, 2.65 mmol), (4-hydroxyphenyl)boronic acid (547 mg, 3.97 mmol) and tetrakis(triphenylphosphine) palladium(O) (153 mg, 0.132 mmol) in DMF (7 mL) was added Na₂CO₃ (1 M, 5.29 mL). The resulting mixture was purged with nitrogen gas before being irradiated in the microwave at 130° C. for 10 min. The reaction mixture was then poured into water and pH was adjusted to pH 6 with acetic acid. The reaction mixture was extracted with EtOAc twice. The combined organic layers were dried over Na₂SO₄, filtered, and concentrated in vacuo. The resulting solid was triturated with a small amount of EtOAc to provide F63-1 as an orange solid (425 mg, 79%). Analytical method 5; t_{R}=0.47 min; [M+H]⁺=203.1.

### Step 2. 4-(2-((Bis(methyl-d3)amino)methyl)-1-methyl-1H-imidazol-5-yl)phenol (F63-2)

To a solution of F63-1 (850 mg, 4.20 mmol) (from two batches) in THF (21 mL) was added deuterated-d₆-dimethylamine HCl salt (736 mg, 8.41 mmol) and TEA (1.758 mL, 12.61 mmol). The resulting mixture was stirred at rt for 2 h, then NaBH(OAc)₃ (1960 mg, 9.25 mmol) and acetic acid (0.963 mL, 16.81 mmol) were added. The reaction mixture was stirred at rt overnight, then quenched with sat. aq. NaHCO₃, and extracted with EtOAc (2×). The combined organic layers were washed with brine, dried over Na₂SO₄, filtered, and concentrated to dryness in vacuo to give a brown solid. The brown solid was triturated in EtOAc to afford F63-2 (906 mg, 91%) as a beige solid. Analytical method 5; t_{R}=0.51 min; [M+H]⁺=238.3.

### Step 3. 2-(4-(2-((Bis(methyl-d3)amino)methyl)-1-methyl-1H-imidazol-5-yl)phenoxy)-4-chlorobenz-aldehyde (F63)

To a mixture of K₂CO₃ (1583 mg, 11.45 mmol) and 4-chloro-2-fluorobenzaldehyde (605 mg, 3.82 mmol) in anhydrous DMF (12 mL) was added F63-2 (906 mg, 3.82 mmol). The resulting mixture was stirred at 90° C. for 4 h then cooled to rt, and diluted with water. The reaction mixture was extracted with EtOAc (2×50 mL). The combined organic phases were washed with water (2×100 mL) and then washed with 1.0 N aq. HCl (2×50 mL). The combined aqueous phases were basified with sat. aqueous K₂CO₃ solution and then extracted with EtOAc (2×100 mL). The combined organic phases were dried over sodium sulfate, filtered, and concentrated to dryness in vacuo to afford F63 (1.22 g, 3.25 mmol, 85% yield) as a clean light brown solid upon standing under vacuum overnight. Analytical method 5; t_{R}=0.99 min; [M+H]⁺=376.1. ¹H NMR (400 MHz, dichloromethane-d₂) b ppm 10.50 (d, J=0.73 Hz, 1H) 7.91 (d, J=8.44 Hz, 1H) 7.49 (d, J=7.78 Hz, 2H) 7.19-7.26 (m, 3H) 7.01 (m, 1H) 7.00 (s, 1H) 3.73 (s, 3H) 3.60 (s, 2H).

### Example 11.3: Synthesis of 4-chloro-2-(4-(1-methyl-2-(pyrrolidin-1-ylmethyl)-1H-imidazol-5-yl)phenoxy)benzaldehyde (F2)

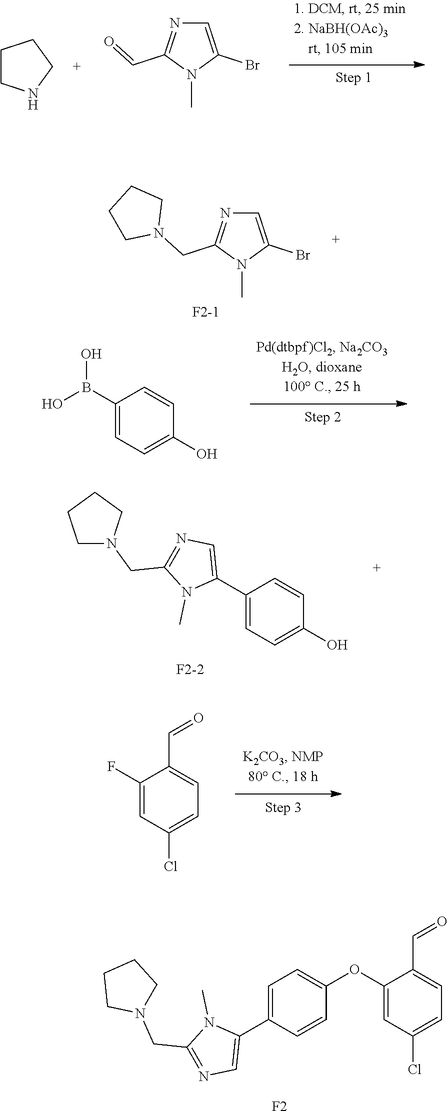

### Step 1. 5-Bromo-1-methyl-2-(pyrrolidin-1-ylmethyl)-1H-imidazole (F2-1)

To a solution of 5-bromo-1-methyl-1H-imidazole-2-carbaldehyde (1.890 g, 10.0 mmol) in DCM (70 mL) was added pyrrolidine (1.643 mL, 20.0 mmol). After stirring for 25 min at rt NaBH(OAc)₃ (8.48 g, 40.0 mmol) was added. The resulting mixture was stirred for 105 min at rt, then concentrated to dryness in vacuo, and partitioned between EtOAc (250 mL) and 1 M aq. NaOH (50 mL). The organic layer was washed with 1 M NaOH (2×40 mL) and brine (20 mL), dried over Na₂SO₄, filtered, and concentrated to dryness in vacuo to afford F2-1 (assumed to be 10.0 mmol) as a yellow solid. The crude product was used in the next step without purification. Analytical method 11; t_{R}=0.66 min; [M+H]⁺=244.1.

### Step 2. 4-(1-Methyl-2-(pyrrolidin-1-ylmethyl)-1H-imidazol-5-yl)phenol (F2-2)

To F2-1 (10 mmol), (4-hydroxyphenyl)boronic acid (2.76 g, 20.0 mmol) and [1,1′-bis(di-tert-butylphosphino)ferrocene]dichloropalladium(II) (0.978 g, 1.50 mmol) were added dioxane (30 mL) and 1 M aq. Na₂CO₃ (30 mL). The reaction mixture was stirred for 4 h at 100° C. under a N₂ atmosphere. An additional amount of (4-hydroxyphenyl)boronic acid (1.379 g, 10.0 mmol) was added and stirring at 100° C. was continued for 135 min. More (4-hydroxyphenyl)boronic acid (1.379 g, 10.0 mmol) and [1,1′-bis(di-tert-butylphosphino)ferrocene]dichloropalladium(II) (0.244 g, 0.375 mmol) were added and stirring at 100° C. was continued for 18.75 h. EtOAc (250 mL) and H₂O (50 mL) were added and the mixture was filtered over Hyflo. The layers were separated and the organic layer was washed with 5% aq. NaHCO₃ (3×40 mL) and brine (40 mL), dried over Na₂SO₄, filtered, and concentrated to dryness in vacuo. The crude product was purified by silica gel flash chromatography (eluent A: EtOAc/MeOH/DIEA (95:5:2), eluent B: EtOAc/MeOH/DIEA (85:15:2)) to give F2-2 (2.28 g, 8.86 mmol, 89% yield, two steps) as a brown solid. Analytical method 11; t_{R}=0.76 min; [M+H]⁺=258.1.

### Step 3. 4-Chloro-2-(4-(1-methyl-2-(pyrrolidin-1-ylmethyl)-1H-imidazol-5-yl)phenoxy)benzaldehyde (F2)

F2-2 (1.029 g, 4 mmol) and 4-chloro-2-fluorobenzaldehyde (0.824 g, 5.20 mmol) were dissolved in NMP (20 mL) and K₂CO₃ (1.437 g, 10.40 mmol) was added. The reaction mixture was stirred for 18 h at 80° C., and then partitioned between EtOAc (125 mL) and H₂O (20 mL). The organic layer was washed with 5% aq. NaHCO₃ solution (3×10 mL) and brine (10 mL), dried with Na₂SO₄, filtered, and concentrated to dryness in vacuo. The crude product was purified by silica gel flash chromatography (eluent A: EtOAc/DIEA (98:2), eluent B: EtOAc/MeOH/DIEA (90:10:2)) to give F2 as a brown oil. Analytical method 10; t_{R}=0.79 min; [M+H]⁺=396.2.

Alternatively, F2 can be synthesized according to the procedure used for F63 (Example 11.2) Starting from 5-bromo-1-methyl-1H-imidazole-2-carbaldehyde, (4-hydroxyphenyl)boronic acid, pyrrolidine and 4-chloro-2-fluorobenzaldehyde.

### Example 11.4: Synthesis of 4-chloro-2-(2,3-difluoro-4-(1-methyl-2-(pyrrolidin-1-ylmethyl)-1H-imidazol-5-yl)phenoxy)-benzaldehyde (F27)

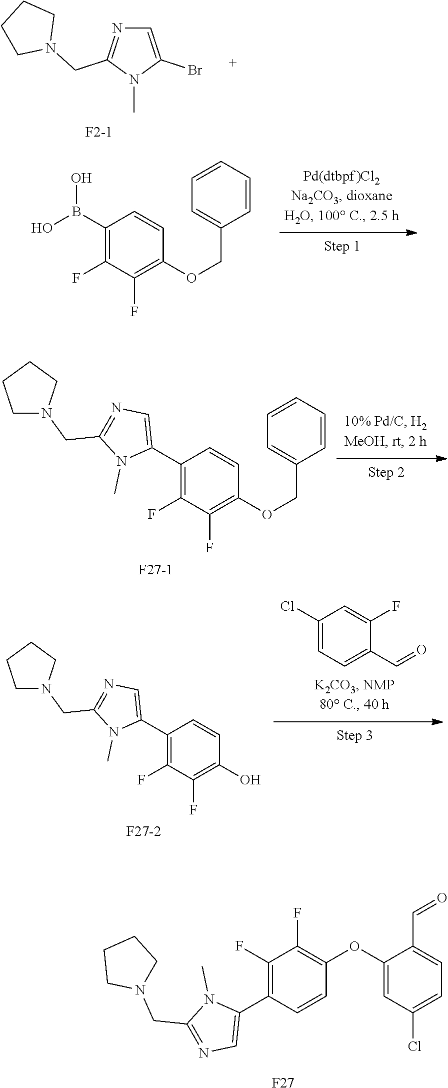

### Step 1. 5-(4-(Benzyloxy)-2,3-difluorophenyl)-1-methyl-2-(pyrrolidin-1-ylmethyl)-1H-imidazole (F27-1)

To F2-1 (325 mg, 1.33 mmol), (4-(benzyloxy)-2,3-difluorophenyl)boronic acid (702 mg, 2.66 mmol) and [1,1′-bis(di-tert-butylphosphino)ferrocene]dichloropalladium(II) (87 mg, 0.133 mmol) dissolved in dioxane (4 mL) was added 1 M aq. Na₂CO₃ (4 mL, 4.0 mmol). The reaction mixture was stirred for 2.5 h at 100° C. under an atmosphere of nitrogen, and then partitioned between EtOAc (70 mL) and H₂O (10 mL). The organic layer was washed with 5% aq. NaHCO₃ (2×10 mL) and brine (5 mL), dried over Na₂SO₄, filtered, and concentrated to dryness. The crude product was purified by flash chromatography over silica gel (eluent A: EtOAc/DIEA (98:2), eluent B: EtOAc/MeOH/DIEA (90:10:2)) to afford F27-1 (387.6 mg, 1.011 mmol, 76% yield). Analytical method 10; t_{R}=0.83 min; [M+H]⁺=384.5.

### Step 2. 2,3-Difluoro-4-(1-methyl-2-(pyrrolidin-1-ylmethyl)-1H-imidazol-5-yl)phenol (F27-2)

To F27-1 (387 mg, 1.009 mmol) dissolved in MeOH (5 mL) was added 10% Pd/C (53.7 mg, 0.050 mmol) and the resulting suspension was stirred for 2 h at rt under an atmosphere of nitrogen. The reaction mixture was filtered over Hyflo and concentrated to dryness in vacuo to give F27-2 (293 mg, 0.999 mmol, 99% yield). Analytical method 10; t_{R}=0.46 min; [M+H]⁺=294.2.

### Step 3. 4-Chloro-2-(2,3-difluoro-4-(1-methyl-2-(pyrrolidin-1-ylmethyl)-1H-imidazol-5-yl)phenoxy)-benzaldehyde (F27)

F27-2 and 4-chloro-2-fluorobenzaldehyde (158 mg, 0.999 mmol) were dissolved in NMP (8 mL) and K₂CO₃ (276 mg, 1.998 mmol) was added. The reaction mixture was stirred for 20 h at 80° C. A additional portion of 4-chloro-2-fluorobenzaldehyde (15.8 mg, 0.099 mmol) was added and stirring was continued for 20 h at 80° C. The reaction mixture was partitioned between EtOAc (70 mL) and H₂O (15 mL). The organic layer was washed with 5% aq. NaHCO₃ (2×10 mL) and brine (5 mL), dried over Na₂SO₄, filtered, and concentrated to dryness in vacuo. The crude product was purified by silica gel flash chromatography (eluent A: EtOAc/DIEA (98:2), eluent B: EtOAc/MeOH/DIEA (90:10:2)) to afford F27 (283 mg, 0.655 mmol, 65.6% yield) as a yellow solid. Analytical method 10; t_{R}=0.85 min; [M+H]⁺=432.2.

### Example 11.5: Synthesis of 4-chloro-2-(4-(2-((dimethylamino)methyl)-1-methyl-1H-imidazol-5-yl)phenoxy)-6-(2,2,2-trifluoroethoxy)benzaldehyde (F34)

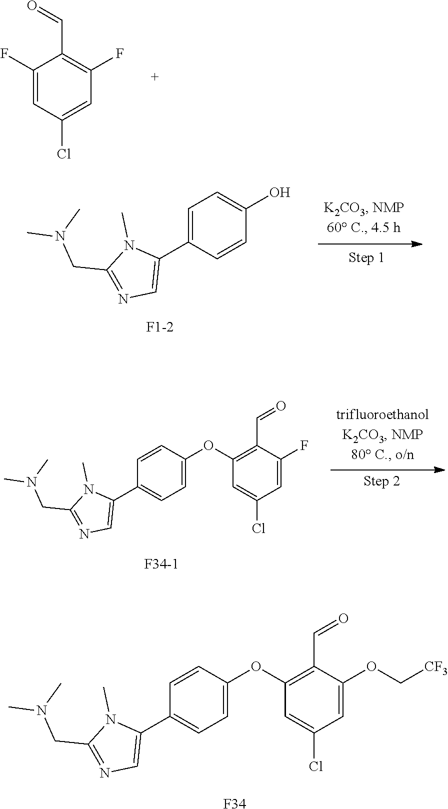

### Step 1. 4-Chloro-2-(4-(2-((dimethylamino)methyl)-1-methyl-1H-imidazol-5-yl)phenoxy)-6-fluorobenz-aldehyde (F34-1)

4-Chloro-2,6-difluorobenzaldehyde (404 mg, 2.288 mmol), F1-2 (529 mg, 2.288 mmol), and K₂CO₃ (949 mg, 6.86 mmol) were taken up in NMP (10 mL) in a round bottom flask charged with a magnetic stir bar. The reaction was heated to 60° C. for 4.5 hours, and then cooled to rt and diluted with EtOAc (100 mL). The organic layer was washed with H₂O (2×75 mL), sat. NaHCO₃(50 mL), and brine (50 mL), dried over Na₂SO₄, filtered, and concentrated in vacuo to provide a crude yellow-green oil (F34-1) which was carried onto the next step without purification. Analytical method 7; t_{R}=0.71 min; [M+H]⁺=388.2.

### Step 2. 4-Chloro-2-(4-(2-((dimethylamino)methyl)-1-methyl-1H-imidazol-5-yl)phenoxy)-6-(2,2,2-trifluoroethoxy)benzaldehyde (F34)

2,2,2-Trifluoroethanol (0.82 mL, 11.44 mmol) was added in one portion via syringe to crude intermediate F34-1 (887 mg, 2.288 mmol) and K₂CO₃ in NMP (10 mL) in a round bottom flask charged with a magnetic stir bar. The reaction was heated to 80° C. under an N₂ atmosphere overnight, and then cooled to rt and diluted with EtOAc (150 m). The organic layer was washed with H₂O (1×100 mL, 1×75 mL, sat. NaHCO₃ (50 m), and brine (50 m), dried with Na₂SO₄, filtered, and concentrated in vacuo to provide a crude dark orange oil. The crude oil was purified by silica gel flash chromatography (40 g column, liquid loading (in 10% MeOH:DCM), eluting with 0-100% EtOAc then 10% MeOH:DCM). The product-containing fractions were combined and concentrated in vacuo to provide F34 (380 mg, 36%) as a beige foam. Analytical method 7; t_{R}=0.81 min; [M+H]⁺=468.1.

### Example 11.6: Synthesis of 4-chloro-2-(4-(2-methoxypyrimidin-5-yl)phenoxy)benzaldehyde (F71)

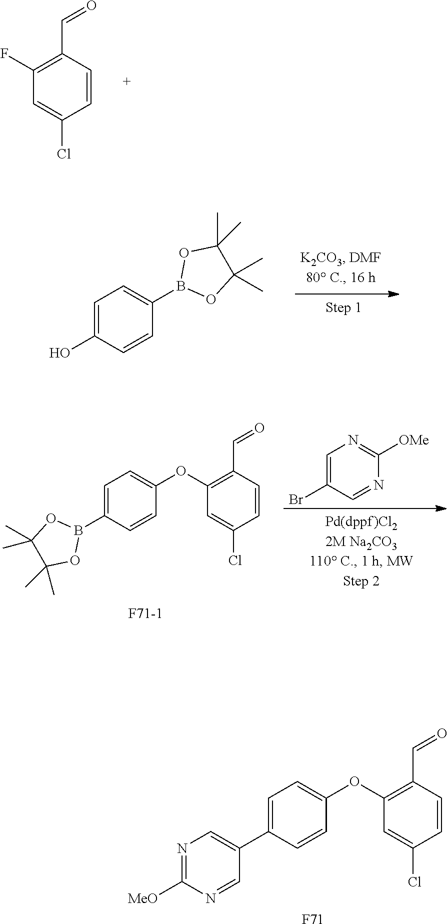

### Step 1: 4-Chloro-2-(4-(4,4,5,5-tetramethyl-1,3,2-dioxaborolan-2-yl)phenoxy)benzaldehyde (F71-1)

To a solution of 4-chloro-2-fluorobenzaldehyde (2.5 g, 15.77 mmol) and 4-hydroxyphenylboronic ester (3.47 g, 15.77 mmol) in DMF (31.5 mL) was added K₂CO₃ (4.36 g, 31.5 mmol) and the resulting mixture was stirred in a 80° C. oil bath. After 16 h at 80° C., the reaction was cooled to rt, diluted with water, and extracted with EtOAc. The combined organic phases were washed with brine, dried over MgSO₄, filtered, and concentrated to afford a crude orange syrup. The crude material was purified via silica gel flash chromatography (50 g column, eluting with 0→25% EtOAc in heptane) to give F71-1 as a white solid (4.27 g, 76%). Analytical method 8; t_{R}=1.77 min; [M+H]⁺=359.0.

### Step 2: 4-Chloro-2-(4-(2-methoxypyrimidin-5-yl)phenoxy)benzaldehyde (F71)

To a microwave vial charged with F71-1 (285 mg, 0.795 mmol), 5-bromo-2-methoxypyrimidine (186 mg, 0.984 mmol) and Pd(dppf)Cl₂-DCM (32.4 mg, 0.040 mmol) was added DMF (2271 μL) and 2 M Na₂CO₃ (795 μL, 1.589 mmol). The vial was capped and sparged with N₂ for 10 min before being stirred at 110° C. for 1 h in a microwave reactor. The reaction mixture was diluted with water and extracted with EtOAc. The combined organic phases were washed with brine (3×), dried over MgSO₄, filtered, and concentrated to dryness to afford the crude product (379 mg) as a dark syrup. The crude material was purified silica gel via flash chromatography (50 g column, eluting with 0→100% EtOAc in heptane) to give F71 (213 mg, 79% yield) as a pale yellow solid. Analytical method 8; t_{R}=1.44 min; [M+H]⁺=341.0.

### Example 11.7: Synthesis of 2-(4-(2-(1-((tert-butyldimethylsilyl)oxy)propyl)-1-methyl-1H-imidazol-5-yl)phenoxy)-4-chlorobenzaldehyde (F68)

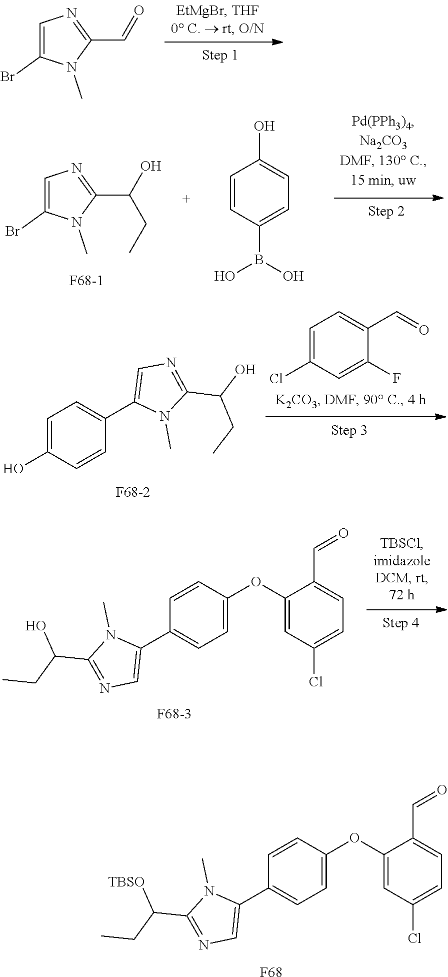

### Step 1. 1-(5-Bromo-1-methyl-1H-imidazol-2-yl)propan-1-ol (F68-1)

EtMgBr (1 M in THF, 2.08 mL, 2.08 mmol) was added dropwise over 1 minute to a solution of 5-bromo-1-methyl-1H-imidazole-2-carbaldehyde (375 mg, 1.984 mmol) in THF (9.9 mL) in a round bottom flask charged with a magnetic stir bar under an atmosphere of N₂ at 0° C. The resulting mixture was allowed to warm to rt and stirred overnight. The reaction mixture was then diluted with sat. NH₄Cl (25 mL), DCM (100 mL), and H₂O (25 mL). The organic phase was separated, and the aqueous layer was washed with DCM (2×50 mL). The combined organic phases were dried with Na₂SO₄, filtered, and concentrated in vacuo to provide a crude yellow oil. The crude oil was purified by silica gel flash chromatography (24 g column, liquid loading (in DCM), eluting with 0-5% MeOH:DCM), but the product-containing fractions contained significant impurities. The fractions were concentrated in vacuo, and the resulting oil was repurified via silica gel flash chromatography (24 g column, liquid loading (in DCM), eluting with 0-50-100% EtOAc:heptane) to provide F68-1 (105 mg, 24% yield) as a colorless oil. Analytical method 5; t_{R}=0.62 min, [M−H]⁻=217.0.

### Step 2. 4-(2-(1-Hydroxypropyl)-1-methyl-1H-imidazol-5-yl)phenol (F68-2)

F68-1 (103 mg, 0.470 mmol) was added as a solution in DMF (2 mL) to (4-hydroxyphenyl)boronic acid (130 mg, 0.940 mmol) and Pd(PPh₃)₄ (27.2 mg, 0.024 mmol) in a 2-5 mL microwave vial charged with a magnetic stir bar. 1 M aqueous Na₂CO₃ (0.940 mL, 0.940 mmol) was added and the resulting slurry was purged with N₂ for 3 min. The vial was capped and the resulting mixture was heated in the microwave for 15 min at 130° C. and then diluted with H₂O (50 mL) and EtOAc (50 mL). The organic phase was separated, and the aqueous phase was washed with EtOAc (2×50 mL). The combined organic phases were washed with H₂O (50 mL), dried with Na₂SO₄, filtered, and concentrated in vacuo to provide a crude red oil. The crude oil was purified via silica gel flash chromatography (24 g column, liquid loading (in DCM plus a few drops of MeOH to aid solubility), eluting with 0→10% MeOH:DCM) to provide F68-2 (104 mg, 95% yield) as a light tan oil. Analytical method 5; t_{R}=0.58 min, [M+H]⁺=233.1.

### Step 3. 4-Chloro-2-(4-(2-(1-hydroxypropyl)-1-methyl-1H-imidazol-5-yl)phenoxy)benzaldehyde (F68-3)

4-Chloro-2-fluorobenzaldehyde (69 mg, 0.437 mmol) was added in one portion to a suspension of F68-2 (102 mg, 0.437 mmol) and K₂CO₃ in DMF (5 mL) in a round bottom flask charged with a magnetic stir bar. The reaction mixture was allowed to stir at 90° C. for 4 hours, and then diluted with EtOAc (75 mL) and washed with H₂O (75 mL). The aqueous phase was washed with EtOAc (75 mL). The combined organic phases were washed with H₂O (50 mL) and brine (50 mL), dried with Na₂SO₄, filtered, and concentrated in vacuo to provide F68-3 (134 mg) as a crude red/tan residue which was carried onto the next step without purification. Analytical method 5; t_{R}=1.09 min; [M+H]⁺=371.1.

### Step 4. 2-(4-(2-(1-((tert-Butyldimethylsilyl)oxy)propyl)-1-methyl-1H-imidazol-5-yl)phenoxy)-4-chlorobenz-aldehyde (F68)

Imidazole (47 mg, 0.690 mmol) was added in one portion to a suspension of F68-3 (128 mg, 0.345 mmol) in DCM (2 mL) in a vial charged with a magnetic stir bar at rt. TBSCl (78 mg, 0.518 mmol) was added in one portion via glass pipette as a solution in DCM (1.5 mL). The resulting mixture was allowed to stir at rt under N₂ over 2 days and then diluted with EtOAc (75 mL). The reaction mixture was washed with sat. NaHCO₃ (50 mL) and brine (40 mL), dried over Na₂SO₄, and concentrated in vacuo to provide a crude pale tan oil. The crude oil was purified via silica gel flash chromatography (24 g column, liquid loading (in DCM), eluting with 0-50% EtOAc:heptane) to provide F68 (141.8 mg, 85% yield) as a colorless oil. Analytical method 5; t_{R}=1.72 min; [M+H]⁺=484.9.

### Example 11.8: Synthesis of 4-(4-(2-((dimethylamino)methyl)-1-methyl-1H-imidazol-5-yl)phenoxy)-6-hydroxynicotin-aldehyde (F76)

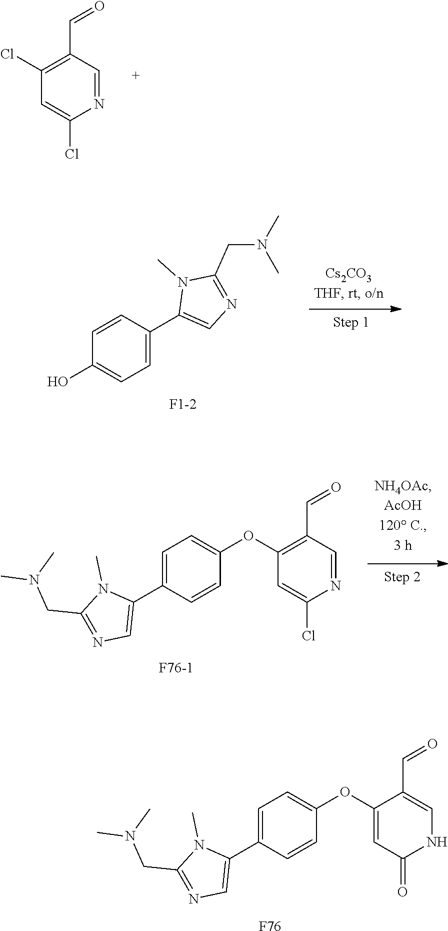

### Step 1. 6-Chloro-4-(4-(2-((dimethylamino)methyl)-1-methyl-1H-imidazol-5-yl)phenoxy)nicotinaldehyde (F76-1)

To a flask containing 4,6-dichloronicotinaldehyde (400 mg, 2.27 mmol), F1-2 (526 mg, 2.27 mmol) and cesium carbonate (740 mg, 2.27 mmol) was added THF (11 mL). The resulting mixture was stirred at rt overnight and then concentrated. Water was added to afford a white slurry. The reaction mixture was filtered and washed with water to afford an off-white solid. The crude product was purified by reverse flash column chromatography (water/ACN, neutral) to afford F76-1 as an off-white powder after freeze-drying the pure fractions (381 mg, 45% yield). Analytical method 5; t_{R}=0.83 min; [M+H]⁺=371.1.

### Step 2. 4-(4-(2-((Dimethylamino)methyl)-1-methyl-1H-imidazol-5-yl)phenoxy)-6-hydroxynicotinaldehyde (F76)

To a flask containing F76-1 (381 mg, 1.03 mmol) and ammonium acetate (396 mg, 5.14 mmol) was added acetic acid (7 mL). The mixture was heated to 120° C. for 3 h, then was cooled to rt, and concentrated to dryness to afford a crude oil. The crude material was purified by reversed-phase flash column chromatography (water/ACN, neutral) to afford F76 as an off-white powder after freeze-drying the pure fractions (53 mg, 15% yield). Analytical method 5; t_{R}=0.51 min; [M+H]⁺=353.2.

### Example 11.9: Synthesis of 5-iodo-1,4-dimethyl-1H-imidazole-2-carbaldehyde (F10-1)

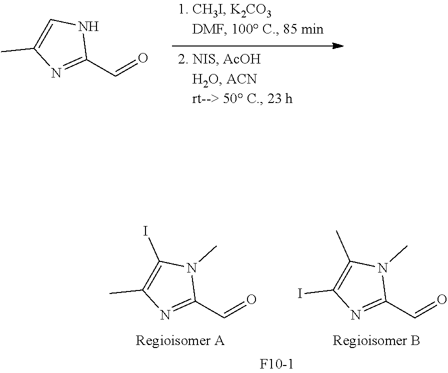

4-Methyl-1H-imidazole-2-carbaldehyde (881 mg, 8.00 mmol) was dissolved in DMF (15 mL) at 100° C. The solution was cooled to rt and K₂CO₃ (1.327 g, 9.60 mmol) and methyl iodide (0.650 mL, 10.40 mmol) were added. The resulting suspension was stirred for 75 min at 100° C. in a closed vessel and then K₂CO₃ (0.332 g, 2.400 mmol) and methyl iodide (0.150 mL, 2.400 mmol) were added. The reaction mixture was stirred for 10 min at 100° C., and then cooled to rt. H₂O (10 mL), AcOH (4.58 mL, 80 mmol) and NIS (1980 mg, 8.80 mmol) were added and the resulting mixture was stirred for 40 min at rt, and then for 13 h 40 min at 50° C. An additional amount of NIS (990 mg, 4.40 mmol) was added and stirring at 50° C. was continued for 5 h. NIS (495 mg, 2.200 mmol) and ACN/H₂O (1:1) (20 mL) were then added and the reaction mixture was stirred for 3 h at 50° C. Further AcOH (2.290 mL, 40.0 mmol) was added and stirring was continued for 40 min at 50° C. ACN was removed in vacuo. EtOAc (150 mL), 4 M aq. NaOH (30 mL) and aq. sodium thiosulfate (3162 mg, 20.00 mmol) were added to the reaction mixture and the phases were separated. The organic phase was washed with 1 M aq. NaOH (3×20 mL) and brine (20 mL), dried over Na₂SO₄, filtered, and concentrated to dryness in vacuo. The crude product was purified by silica gel flash chromatography (eluent A: DCM; eluent B: DCM/MeOH (95:5)) to afford F10-1 (808 mg, 3.23 mmol, 40.4% yield) (mixture of regioisomers) as a light yellow solid. Analytical method 11; Regioisomer A: t_{R}=1.09 min; [M+H]⁺=251.0; Regioisomer B: t_{R}=1.07 min; [M+H]⁺=251.1.

### Example 11.10: Synthesis of tert-butyl 3-bromo-7,8-dihydro-1,6-naphthyridine-6(5H)-carboxylate (F13-1)

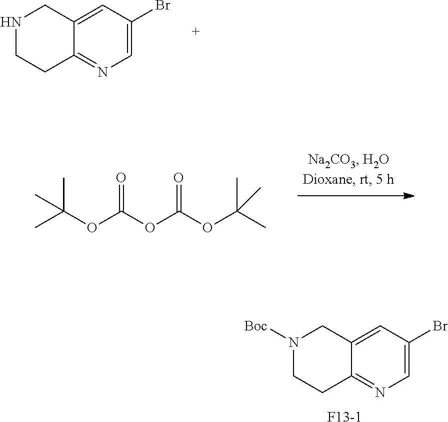

To 3-Bromo-5,6,7,8-tetrahydro-1,6-naphthyridine HCl (499 mg, 2.00 mmol) dissolved in dioxane (15 mL) and 1 M aq. Na₂CO₃ (6.0 mL, 6.00 mmol) was added a solution of Boc₂O (458 mg, 2.100 mmol) in dioxane (5 mL) and the resulting mixture was stirred for 5 h at rt. The reaction mixture was partitioned between EtOAc (50 mL) and 5% aq. NaHCO₃ (20 mL). The organic phase was washed with 5% aq. NaHCO₃ (2×10 mL), 5% aq. KHSO₄ (3×10 mL), 5% aq. NaHCO₃ (10 mL) and brine (10 mL), dried over Na₂SO₄, filtered, and concentrated to dryness in vacuo to afford F13-1 (692 mg, 2.99 mmol, 100% yield). The crude product was used in the next step without purification. Analytical method 10; t_{R}=1.08 min; [M+H]⁺=313.2.

TABLE 26

| BB | | | Synthesis procedure/ |
|---|---|---|---|
| No. | Structure | Name | LCMS |
| F15-1 | 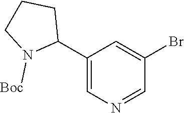 [SMILES: CC(C)(C)OC(=O)N1CCCC1c1cncc(Br)c1] | tert-butyl 2-(5-bromopyridin- 3-yl)pyrrolidine-1- carboxylate (racemate) | F13-1 (Example 11.10)- starting from 3-bromo-5- (pyrrolidin-2-yl)pyridine/ Analytical method 10 t_{R} = 1.10 min [M + H]⁺ = 327.1 |

### Example 11.11: Synthesis of 4-chloro-2-((5-(2-((dimethylamino)methyl)-1-methyl-1H-imidazol-5-yl)pyridin-2-yl)oxy)benzaldehyde trifluoroacetate (F77)

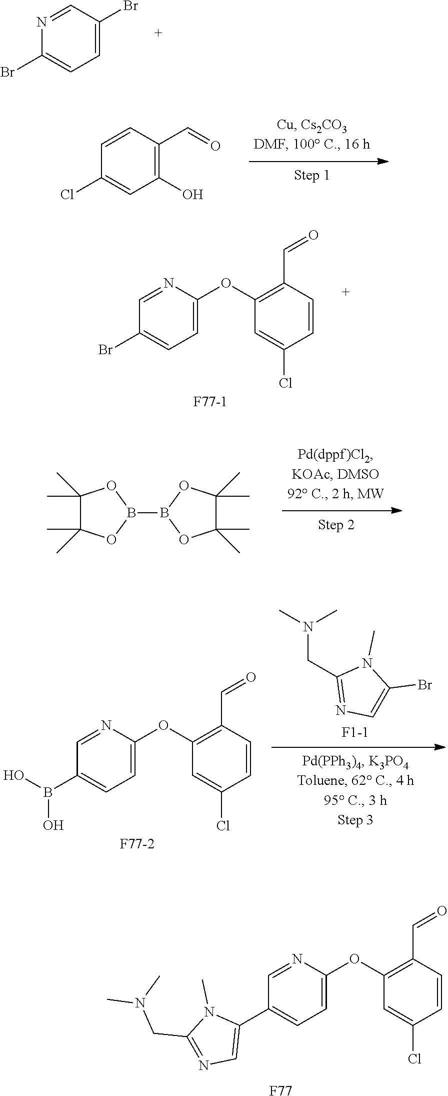

### Step 1. 2-((5-Bromopyridin-2-yl)oxy)-4-chlorobenzaldehyde (F77-1)

2,5-Dibromopyridine (1 g, 4.22 mmol), 4-chloro-2-hydroxybenzaldehyde (0.661 g, 4.22 mmol), copper (0.107 g, 1.689 mmol) and cesium carbonate (2.063 g, 6.33 mmol) in DMF (5 mL) under a N₂ atmosphere was heated at 100° C. for 16 h. The reaction mixture was filtered and directly purified by normal phase silica gel chromatography (120 g column, eluting with 30→70% EtOAc in heptane) to afford intermediate F77-1 (0.24 g, 18% yield). Analytical method 5; t_{R}=1.11 min; [M+H]⁺=311.6. ¹H NMR (400 MHz, Chloroform-d) δ 10.22 (s, 1H), 8.23 (d, J=2.4 Hz, 1H), 7.93 (d, J=8.4 Hz, 1H), 7.90 (dd, J=8.6, 2.6 Hz, 1H), 7.35 (dd, J=8.7, 1.6 Hz, 1H), 7.22 (d, J=1.9 Hz, 1H), 7.04 (d, J=8.4 Hz, 1H).

### Step 2. (6-(5-Chloro-2-formylphenoxy)pyridin-3-yl)boronic acid (F77-2)

A mixture of pinacol borane (0.390 g, 1.536 mmol), F77-1 (0.24 g, 0.768 mmol), Pd(dppf)Cl₂ (0.056 g, 0.077 mmol) and KOAc (0.151 g, 1.536 mmol) in dry DMSO (2 mL) was microwaved at 92° C. for 2 h under an atmosphere of N₂. The reaction mixture was filtered to provide F77-2 (crude) which was used in next step without purification. Analytical method 5; t_{R}=0.77 min; [M+H]⁺=277.9.

### Step 3. 4-Chloro-2-((5-(2-((dimethylamino)methyl)-1-methyl-1H-imidazol-5-yl)pyridin-2-yl)oxy)benzaldehyde trifluoroacetate (F77)

A microwave vial was charged with Pd(PPh₃)₄(83 mg, 0.072 mmol), F1-1 (189 mg, 0.865 mmol), F77-2 (200 mg, 0.721 mmol), tripotassium phosphate (2 M aq. solution, 1.081 mL, 2.162 mmol) and toluene (6 mL) and placed under an atmosphere of N₂. The resulting mixture was microwaved at 62° C. for 4 h and 95° C. for 3 h. The crude product was purified by reverse phase chromatography (40 g C18 column, eluting with 0-100% MeCN in water, with 0.1% TFA) to afford product F77 (70 mg, 26% yield) as a TFA salt. Analytical method 7; t_{R}=0.90 min; [M+H]+=370.8.

### Example 11.12: Synthesis of N-(2-((tert-butyldimethylsilyl)oxy)ethyl)-1-(4-(5-chloro-2-formylphenoxy)-phenyl)-1H-imidazole-4-carboxamide (F57)

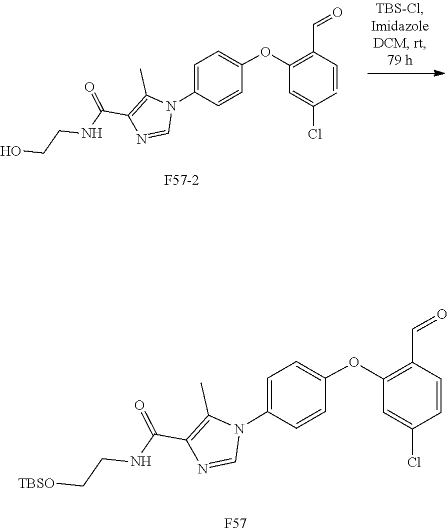

To F57-2 (57 mg, 0.142 mmol) (see Table 25) suspended in DCM (2 mL) was added TBS-Cl (34 mg, 0.226 mmol) and imidazole (21 mg, 0.308 mmol) and the resulting mixture was stirred rt for 63 h. Additional DCM (2 mL), TBS-Cl (11 mg, 0.0729 mmol), and imidazole (8 mg, 0.117 mmol) were added. DMF (3 drops) was then added and the resulting mixture was stirred for 16 h at rt and then concentrated. Purification via normal phase chromatography on a silica column eluting with 0-100% ethyl acetate in heptane provided F57 (28 mg, 36%) as an off-white foam in 94% purity. Analytical method 4; t_{R}=3.30 min; [M+H]⁺=514.1 ¹H NMR (400 MHz, CD₂Cl₂) δ 10.32 (d, J=0.7 Hz, 1H), 7.82 (d, J=8.4 Hz, 1H), 7.66 (s, 1H), 7.31-7.24 (m, 2H), 7.21-7.13 (m, 3H), 6.94 (d, J=1.9 Hz, 1H), 3.69 (t, J=5.9 Hz, 2H), 3.42 (q, J=5.9 Hz, 2H), 2.43 (s, 3H), 0.82 (s, 9H).

### Example 11.13: Synthesis of (R)-1-(5-bromo-1-methyl-1H-imidazol-2-yl)-N,N-dimethylethan-1-amine (F50-4)

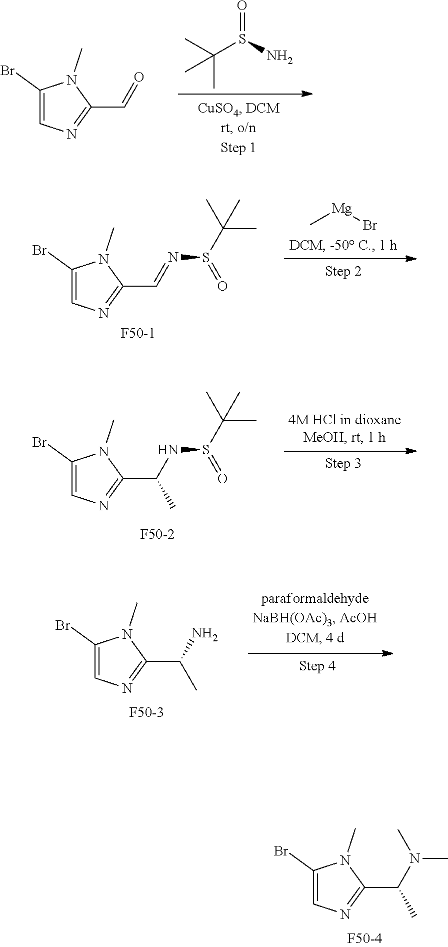

### Step 1. (R,E)-N-((5-Bromo-1-methyl-1H-imidazol-2-yl)methylene)-2-methylpropane-2-sulfinamide (F50-1)

To 5-bromo-1-methyl-1H-imidazole-2-carbaldehyde (3 g, 15.87 mmol) in anhydrous DCM (40 mL) was added (R)-2-methylpropane-2-sulfinamide (2.116 g, 17.46 mmol) and copper sulfate anhydrous (5.07 g, 31.7 mmol). The resulting mixture was stirred at rt overnight and then filtered through a pad of Celite®, flushing with DCM. The filtrate was concentrated via rotary evaporation and purified by normal phase flash chromatography, using a silica gel column, eluting with 0-100% EtOAc in heptane to afford F50-1 (4 g, 13.69 mmol, 86% yield) as a white solid. Analytical method 5; t_{R}=0.83 min; [M+H]⁺=294.2.

### Step 2. (R)—N—((R)-1-(5-Bromo-1-methyl-1H-imidazol-2-yl)ethyl)-2-methylpropane-2-sulfinamide (F50-2)

To a solution of F50-1 (2.7 g, 9.24 mmol) in anhydrous DCM (50 mL) at −50° C. was added 3 M MethylMgBr in ether (7.08 mL, 21.25 mmol) via a syringe. The resulting mixture was stirred at −50° C. for 1 h and then being quenched with sat. NH₄Cl. After warming to rt, the reaction mixture was diluted with sat. NaHCO₃ (50 mL) and extracted with 2×60 mL of DCM. The separated organic phases were dried with sodium sulfate, filtered, and concentrated via rotovap to afford a white solid. This solid was recrystallized in EtOAc/heptane to afford F50-2 (2.03 g, 6.59 mmol, 71% yield) as a white power. Analytical method 5; t_{R}=0.68 min; [M+H]⁺=310.1.

### Step 3. (R)-1-(5-Bromo-1-methyl-1H-imidazol-2-yl)ethanamine hydrochloride (F50-3)

To a suspension of F50-2 (2.02 g, 6.55 mmol) in anhydrous MeOH (10 mL) was added 4 M HCl in dioxane (6.55 mL, 26.2 mmol). The resulting mixture turned clear and was stirred at rt for 1 h. Diethyl ether (100 mL) was then added. The resulting white precipitate was collected by vacuum filtration to give F50-3 (1.815 g, 6.55 mmol, 100% yield) as white power. Analytical method 5; t_{R}=0.51 min; [M+H]⁺=203.9.

### Step 4. (R)-1-(5-Bromo-1-methyl-1H-imidazol-2-yl)-N,N-dimethylethanamine (F50-4)

To F50-3 (1.815 g, 6.55 mmol) was added anhydrous DCM (50 mL,), paraformaldehyde (1.967 g, 65.5 mmol), NaBH(OAc)₃ (3.47 g, 16.38 mmol), and AcOH (5 mL). The resulting mixture was stirred at rt over 2 days. An additional amount of NaBH(OAc)₃ (1600 mg) and paraformaldehyde (600 mg) were added. The reaction mixture was stirred overnight, then quenched with 100 mL of 2 N Na₂CO₃ solution and extracted with 2×100 mL of DCM. The separated organic phases were dried with sodium sulfate, filtered, and concentrated via rotovap to afford F50-4 (1.46 g, 3.14 mmol, 48.0% yield) as a yellow oil. Minor impurities were present. The material was carried onto the next step without purification. Analytical method 5; t_{R}=0.66 min; [M+H]⁺=233.9.

TABLE 27
Intermediates in Table 27 below were prepared according to the
procedure used for F50-4 (Example 11.13)

| BB | | |
|---|---|---|
| No. | Structure | Synthesis procedure/LCMS/NMR |
| F52-1 | 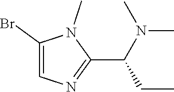 (R)-1-(5-bromo-1-methyl-1H- imidazol-2-yl)-N,N-dimethyl- propan-1-amine | F50-4 (Example 11.13)/Analytical method 5 t_{R} = 0.72 min [M + H]⁺ = 246.2 |
| F80-2 | 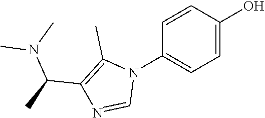 [SMILES: Cc1c([C@@H](C)N(C)C)ncn1-c1ccc(O)cc1] R)-4-(4-(1-(dimethylamino)ethyl)-5-methyl- 1H-imidazol-1-yl)phenol | F50-4 (Example 11.13)-starting from F80- 1 (Example 11.31). Upon treatment with 4 M HCl in dioxane in Step 3 the TBS group was cleaved/¹H NMR (400 MHz, CDCl₃ + D₂O): δ 8.30 (s, 1H), 7.77 (s, 1H), 7.24 (d, J = 8.8 Hz, 2H), 6.93 (d, J = 8.3 Hz, 2H), 4.48-4.44 (m, 1H), 2.65 (s, 6H), 2.13 (s, 3H), 1.57 (d, J = 6.3 Hz, 3H) |

### Example 11.14: Synthesis of (R)-5-bromo-1-methyl-2-(1-(pyrrolidin-1-yl)ethyl)-1H-imidazole (F53-1)

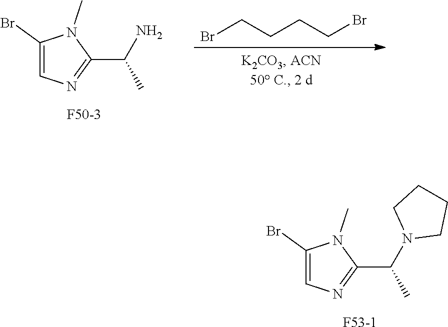

To a solution of 1,4-dibromobutane (468 mg, 2.166 mmol) and K₂CO₃ (998 mg, 7.22 mmol) in 10 mL of anhydrous ACN was added a suspension of F50-3 (400 mg, 1.444 mmol) in anhydrous ACN (5 mL). The resulting mixture was stirred at rt for 30 min before being heated to 50° C. and stirred for 2 days. LCMS showed ˜83% completion of the reaction and a desired product peak was observed. The crude reaction mixture was filtered. The filtrate was diluted with 50 mL of EtOAc and extracted with 50 mL 1.0 N HCl aq. solution. The aqueous phase was washed with EtOAc, basified with 1 N K₂CO₃ solution, and extracted with DCM (50 mL) and EtOAc (50 mL). The combined organic layers were dried over sodium sulfate, filtered, and concentrated via rotary evaporation to afford F53-1 (300 mg, 1.162 mmol, 80% yield) as a colorless oil mixed with white precipitate, which was used in the next step without purification. Analytical method 5; t_{R}=0.80 min; [M+H]⁺=258.0.

### Example 11.15: Synthesis of 5-bromo-1-methyl-2-vinyl-1H-imidazole (F54-1)

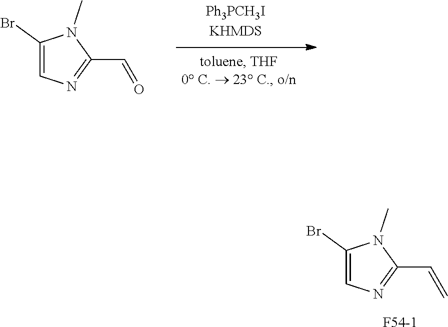

To Ph₃PCH₃I (3.21 g, 7.94 mmol) in anhydrous THF (30 mL) at 0° C. under an atmosphere of nitrogen was added 0.5 M KN(TMS)₂ in toluene (17.99 mL, 8.99 mmol). The resulting yellow mixture was stirred at 0° C. for 30 min and a solution of 5-bromo-1-methyl-1H-imidazole-2-carbaldehyde (1 g, 5.29 mmol) in anhydrous THF (30 mL) was then added dropwise. The resulting mixture was allowed to warm to rt and stirred overnight. The reaction mixture was quenched with sat. NaHCO₃ solution and extracted with 2×100 mL of EtOAc. The combined organic phases were dried over sodium sulfate, concentrated via rotovap, and purified by silica gel flash chromatography (80 g column, eluting with 0→100% EtOAc in heptane) to afford F54-1 (280 mg, 1.497 mmol, 28.3% yield). Analytical method 5; t_{R}=0.68 min; [M+H]⁺=187.0. TLC: R_{f}=0.35, EtOAc/heptane (1:1).

### Example 11.16: Synthesis of 1-(5-bromo-1-methyl-1H-imidazol-2-yl)ethan-1-ol (F70-1)

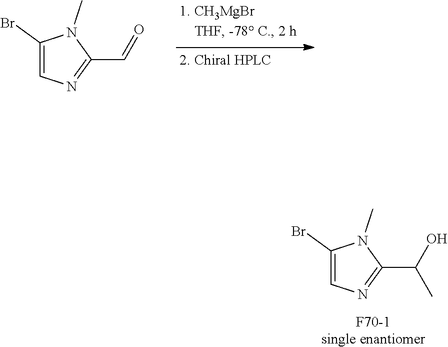

To a solution of 5-bromo-1-methyl-1H-imidazole-2-carbaldehyde (1 g, 5.29 mmol) in anhydrous THF (20 mL) at −78° C. under the presence an atmosphere of nitrogen was added 3 M methylmagnesium bromide in diethyl ether (2.65 mL, 7.94 mmol) dropwise via a syringe. The reaction mixture was stirred at −78° C. for 2 h and then quenched with methanol and sat. NH₄Cl. The resulting mixture was extracted with 3×40 mL of DCM. The combined organic phases were dried over MgSO₄, filtered, and concentrated via rotary evaporation. The formed precipitate was diluted with 2 mL of DCM and filtered to afford the racemic F70-1 (550 mg, 2.68 mmol, 50.7% yield) as a white solid. This racemate was purified by chiral HPLC and F70-1 (260 mg, 1.268 mmol, 24% yield) was obtained (the first peak from chiral separation). Analytical method 5; t_{R}=0.50 min; [M+H]⁺=205.1.

TABLE 28
F69-1 in Table 28 below was prepared according to the procedure
used for F70-1 (Example 11.16).

| BB No. | Structure | Name | LCMS |
|---|---|---|---|
| F69-1 | 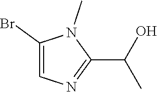 | 1-(5-bromo-1-methyl-1H- imidazol-2-yl)ethan-1-ol (racemate) | Analytical method 5 t_{R} = 0.50 min [M + H]⁺ = 205.1 |

### Example 11.17: Synthesis of 5-bromo-2-ethoxy-1-methyl-1H-imidazole (F73-1)

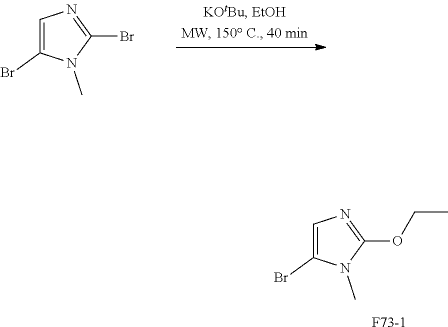

To a microwave vial containing 2,5-dibromo-1-methyl-1H-imidazole (620 mg, 2.58 mmol) and potassium tert-butoxide (870 mg, 7.75 mmol) was added absolute EtOH (6.2 mL). The vial was sealed and heated to 150° C. for 40 min inside a microwave reactor to complete the reaction. The reaction mixture was cooled to rt and concentrated. The crude material was taken up in EtOAc, washed with saturated sodium bicarbonate and brine, and dried over sodium sulfate, filtered, and concentrated. The crude material was purified by normal phase flash chromatography using a silica gel column eluting with 0-100% EtOAc in DCM to afford F73-1 as a pale yellow oil after concentrating the pure fractions (832 mg, 97% yield). Analytical method 8; t_{R}=1.04 min; [M+H]⁺=207.1. The material was cautiously dried under vacuum due to potential low boiling point.

The imidazole building blocks shown in Table 29 were prepared according to the procedure used for F73-1 (Example 11.17).

TABLE 29

| BB No. | Structure | Name | LCMS |
|---|---|---|---|
| F74-1 | 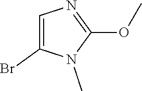 | 5-bromo-2-methoxy-1- methyl-1H-imidazole | Analytical method 7 t_{R} = 0.39 min [M + H]⁺ = 192.9 |
| F75-1 | 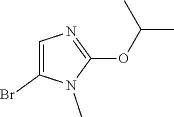 | 5-bromo-2-isopropoxy- 1-methyl-1H-imidazole | Analytical method 5 t_{R} = 1.16 min [M + H]⁺ = 219.1 |

### Example 11.18: Synthesis of 2-(5-bromopyridin-3-yl)-1,4-dimethylpiperazine (F16-2)

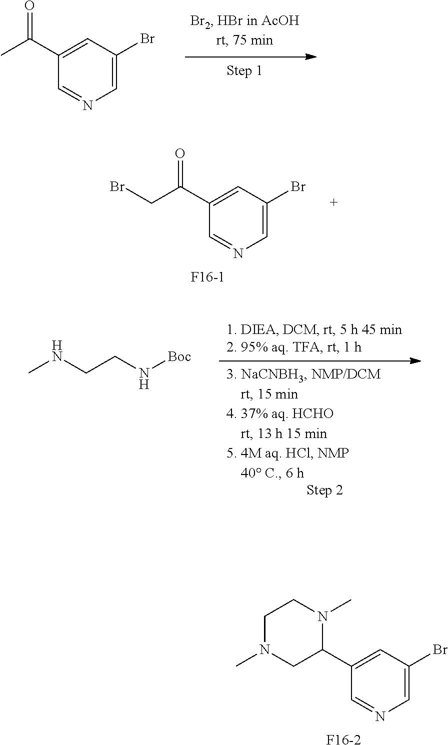

### Step 1. 2-Bromo-1-(5-bromopyridin-3-yl)ethan-1-one (F16-1)

3-Acetyl-5-bromopyridine (1.000 g, 5.00 mmol) was dissolved in 5.7 M HBr in AcOH (10 mL, 57.0 mmol) and Br₂ (0.258 mL, 5.00 mmol) was added dropwise. The reaction mixture was stirred for 75 min at rt and then filtered. The collected solid was partitioned between EtOAc (70 mL) and 5% aq. NaHCO₃ (20 mL). The organic phase was washed with 5% aq. NaHCO₃ (2×10 mL) and brine (10 mL), dried over Na₂SO₄, filtered, and concentrated to dryness in vacuo to afford F16-1 (1.20 g, 4.30 mmol, 86% yield) as an off-white solid. Analytical method 10; t_{R}=0.86 min; [M+H]⁺=278.0.

### Step 2. 2-(5-Bromopyridin-3-yl)-1,4-dimethylpiperazine (F16-2)

To F16-1 (279 mg, 1.000 mmol) dissolved in DCM (10 mL) was added a solution of tert-butyl (2-(methylamino)ethyl)carbamate (192 mg, 1.100 mmol) in DCM (10 mL) and DIEA (0.524 mL, 3.00 mmol). The resulting solution was stirred for 5 h 45 min at rt under a N₂ atmosphere. 95% aq. TFA (20 mL) was then added. The reaction mixture was stirred for 1 h at rt and then concentrated to dryness in vacuo. The obtained residue was suspended in toluene and the mixture concentrated to dryness in vacuo twice.

The residue was dissolved in NMP/DCM (1:1) (10 mL) and NaCNBH₃ (126 mg, 2.000 mmol) was added. The reaction was stirred for 15 min at rt.

37% HCHO (0.243 mL, 3.00 mmol) was then added and stirring at rt was continued for 13.5 h. DCM was removed in vacuo.

NMP (5 mL) and 4 M aq. HCl (10 mL) were added to the obtained residue and the resulting mixture was stirred for 6 h at 40° C. and then concentrated to dryness in vacuo. The obtained residue was partitioned between EtOAc (30 mL) and H₂O (20 mL). The organic phase was discarded. The aqueous phase was basified with 4 M NaOH (10 mL) and the product was extracted with EtOAc (1×40 mL, 2×20 mL). The combined organic phases were washed with brine (10 mL), dried over Na₂SO₄, filtered, and concentrated to dryness in vacuo to afford F16-2 (217 mg, 0.803 mmol, 80% yield) as a brown oil. The crude product was used in the next step without purification. Analytical method 10; t_{R}=0.45 min; [M+H]⁺=270.1

### Example 11.19: Synthesis of 3-bromo-8-(pyrrolidin-1-ylmethyl)imidazo[1,2-a]pyridine (F17-2)

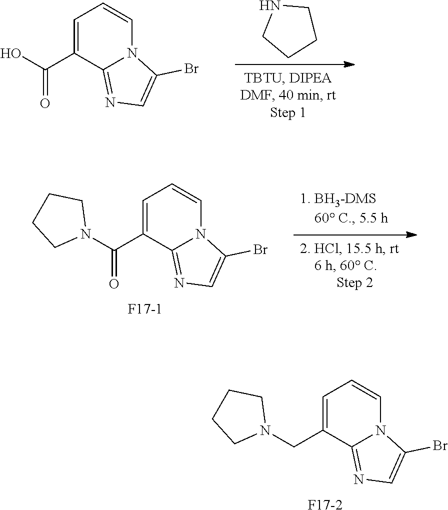

### Step 1. (3-Bromoimidazo[1,2-a]pyridin-8-yl)(pyrrolidin-1-yl)methanone F17-1

A solution of 3-bromo-imidazo[1,2-a] pyridine-8-carboxylic acid (362 mg, 1.5 mmol), TBTU (530 mg, 1.650 mmol) and DIPEA (0.288 mL, 1.650 mmol) in DMF (5 mL) was stirred at rt for 10 min. Pyrrolidine (0.136 mL, 1.650 mmol) was then added and stirring was continued for 40 min at rt. The reaction mixture was partitioned between EtOAc (50 mL) and 5% aq. NaHCO₃ (10 mL). The organic phase was washed with 5% aq. NaHCO₃ solution (2×10 mL) and brine (10 mL), dried over Na₂SO₄, filtered, and concentrated to dryness in vacuo to afford F17-1 (440 mg, 1.301 mmol, 87% yield) as a brown oil which was used in the next step without purification. Analytical method 10; t_{R}=0.69 min; [M+H]⁺=294.0.

### Step 2. 3-Bromo-8-(pyrrolidin-1-ylmethyl)imidazo[1,2-a]pyridine F17-2

To F17-1 (440 mg, 1.301 mmol) dissolved in THF (10 mL) was added borane-dimethyl sulfide complex (371 μL, 3.90 mmol) dropwise at rt and the resulting mixture was stirred at 60° C. for 5.5 h. 1 M aq. HCl (10 mL) was added and the reaction mixture was stirred for 15.5 h at rt, and then 6 h at 60° C. The reaction mixture was partitioned between EtOAc (50 mL) and 5% aq. Na₂CO₃ (10 mL). The organic phase was washed with 5% aq. Na₂CO₃ (2×5 mL) and brine (5 mL), dried over Na₂SO₄, filtered, and concentrated to dryness in vacuo to give F17-2 (285 mg, 78% yield) as a yellow oil which was used in the next step without purification. Analytical method 10; t_{R}=0.47 min; [M+H]⁺=280.1.

### Example 11.20: Synthesis of 5-bromo-1-methyl-2-(1-(pyrrolidin-1-yl)ethyl)-1H-imidazole (F31-2)

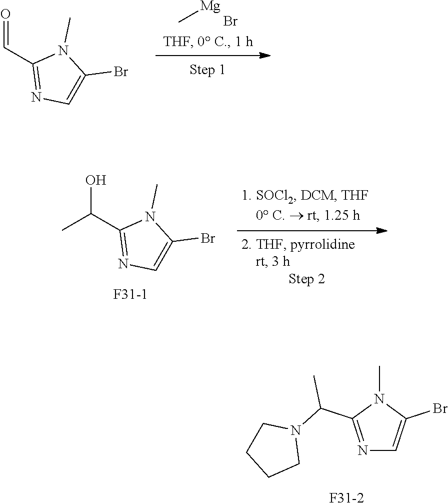

### Step 1. 1-(5-Bromo-1-methyl-1H-imidazol-2-yl)ethan-1-ol (F31-1)

To 5-bromo-1-methyl-1H-imidazole-2-carbaldehyde (567 mg, 3.0 mmol) in THF (15 mL) at 0° C. was added 3 M methylmagnesium bromide in diethyl ether (1.100 mL, 3.30 mmol) dropwise during 10 min. The resulting mixture was stirred for 45 min at 0° C., an additional amount of 3 M methylmagnesium bromide in diethyl ether (0.20 mL, 0.60 mmol) was added and stirring was continued for additional 15 min at 0° C. The reaction mixture was concentrated to dryness in vacuo. The obtained residue was partitioned between EtOAc (70 mL) and 5% aq. NaHCO₃ (15 mL). The organic phase was washed with 5% aq. NaHCO₃ (2×10 mL). The combined aqueous phases were washed with EtOAc (7×15 mL). Then, the combined organic phases were washed with brine (10 mL), dried over Na₂SO₄, filtered, and concentrated to dryness in vacuo to give F31-1 (assumed to be 3.0 mmol). The crude product was used in the next step without purification. Analytical method 11; t_{R}=0.58 min; [M+H]⁺=205.1.

### Step 2. 5-Bromo-1-methyl-2-(1-(pyrrolidin-1-yl)ethyl)-1H-imidazole (F31-2)

Step 2-1. To a solution of F31-1 (2.9 mmol) in DCM/THF (1:1) (12 mL) at 0° C. was added SOCl₂ (0.684 mL, 9.38 mmol). The reaction mixture was allowed to warm up to rt, stirred for 1.25 h, then concentrated to dryness in vacuo.

Step 2-2. The residue from Step 2-1 was dissolved in a mixture of THF (6 mL) and pyrrolidine (2.59 mL, 31.3 mmol). The resulting mixture was stirred for 3 h at rt and then concentrated to dryness in vacuo. The obtained residue was partitioned between EtOAc (70 mL) and 10% aq. Na₂CO₃ (10 mL). The organic phase was washed with 10% aq. Na₂CO₃ (2×5 mL). The combined aqueous phases were washed with EtOAc (6×15 mL). All organic phases were combined, washed with brine (5 mL), dried over Na₂SO₄, filtered, and concentrated to dryness in vacuo to afford F31-2 (715 mg, 2.77 mmol, 95% yield). The crude product was used in the next step without purification. Analytical method 11; t_{R}=0.75 min; [M+H]⁺=258.1.

### Example 11.21: Synthesis of 4-(1-methyl-2-(pent-1-yn-1-yl)-1H-imidazol-5-yl)phenol (F18-4)

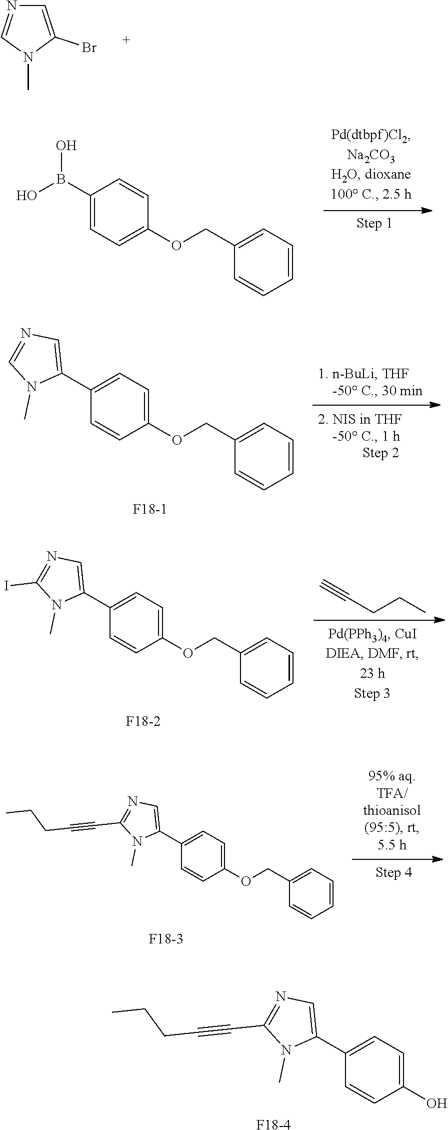

### Step 1. 5-(4-(Benzyloxy)phenyl)-1-methyl-1H-imidazole (F18-1)

To (4-(benzyloxy)phenyl)boronic acid (2.281 g, 10.00 mmol), 5-bromo-1-methyl-1H-imidazole (1.610 g, 10.00 mmol) and [1,1′-bis(di-tert-butylphosphino)ferrocene]dichloropalladium(II) (0.326 g, 0.500 mmol) were added dioxane (25 mL) and 1 M aq. Na₂CO₃ (25 mL, 25.00 mmol). The resulting mixture was stirred for 2.5 h at 100° C. in a N₂ atmosphere and then partitioned between EtOAc (200 mL) and H₂O (20 mL). The organic phase was washed with 5% aq. NaHCO₃ (3×25 mL) and brine, dried over Na₂SO₄, filtered, and concentrated to dryness in vacuo. The crude product was purified by silica gel flash chromatography (eluent A: EtOAc/DIEA (98:2); eluent B: EtOAc/MeOH/DIEA (90:10:2)) to afford F18-1 (1.173 g, 4.44 mmol, 44% yield). Analytical method 10; t_{R}=0.75 min; [M+H]⁺=265.2.

### Step 2. 5-(4-(Benzyloxy)phenyl)-2-iodo-1-methyl-1H-imidazole (F18-2)

F18-1 (538 mg, 2.035 mmol) was dissolved in THF (50 mL). The brown solution was cooled down to −50° C. and placed under a N₂ atmosphere turning into a brown suspension. 1.6 M n-BuLi in hexane (1.781 mL, 2.85 mmol) was added dropwise over 10 min at −50° C. After 20 min of stirring at −50° C., an additional amount of 1.6 M n-BuLi in hexane (0.509 mL, 0.814 mmol) was added dropwise over 5 min. The brown suspension turned into a red solution. After 10 min of stirring at −50° C., N-iodosuccinimide (641 mg, 2.85 mmol) in THF (5 mL) was added dropwise over 10 min. The reaction mixture was stirred for 1 h at −50° C. and then quenched by addition of H₂O (10 mL). THF was removed in vacuo and the residue was partitioned between EtOAc (80 mL) and 5% aq. NaHCO₃ (10 mL). The organic phase was washed with 5% aq. NaHCO₃ (2×5 mL). The combined aqueous phases were washed with EtOAc (2×20 mL). The combined organic phases were washed with brine (15 mL), dried over Na₂SO₄, filtered, and concentrated to dryness in vacuo. The crude product was purified by silica gel flash chromatography (eluent A: heptane/DIEA (98:2); eluent B: EtOAc/DIEA (98:2)) to yield F18-2 (372 mg, 0.953 mmol, 47% yield) as a beige solid. Analytical method 10; t_{R}=1.12 min; [M+H]⁺=391.0.

### Step 3. 5-(4-(Benzyloxy)phenyl)-1-methyl-2-(pent-1-yn-1-yl)-1H-imidazole (F18-3)

To F18-2 (293 mg, 750 μmol), tetrakis(triphenylphosphine)palladium (87 mg, 75 μmol) and CuI (14.28 mg, 75 μmol) dissolved in DMF (20 mL) under an atmosphere of argon was added DIEA (0.393 mL, 2250 μmol) and pent-1-yne (0.110 mL, 1125 μmol) and the resulting mixture was stirred for 2.5 h at rt under an atmosphere of argon. An additional amount of pent-1-yne (0.037 mL, 375 μmol) was added and stirring was continued for 1 h at rt under argon. An additional portion of tetrakis(triphenylphosphine)palladium (87 mg, 75 μmol), CuI (14.28 mg, 75 μmol) and pent-1-yne (0.110 mL, 1125 μmol) were added. The reaction mixture was stirred for additional 19.5 h at rt and then partitioned between EtOAc (60 mL) and H₂O (10 mL). The organic phase was washed with 5% aq. NaHCO₃ (3×5 mL) and brine (5 mL), dried over Na₂SO₄, filtered, and concentrated to dryness in vacuo. The crude product was purified by silica gel flash chromatography (eluent A: heptane/DIEA (98:2); eluent B: EtOAc/DIEA (98:2)) to give F18-3 (196 mg, 593 μmol, 79% yield) as a brown oil. Analytical method 10; t_{R}=1.19 min; [M+H]⁺=331.3.

### Step 4. 4-(1-Methyl-2-(pent-1-yn-1-yl)-1H-imidazol-5-yl)phenol (F18-4)

F18-3 (196 mg, 0.593 mmol) was dissolved in 95% aq. TFA/thioanisole (95:5) (5 mL) and the resulting mixture stirred for 5.5 h at rt and then concentrated to dryness in vacuo. The obtained residue was partitioned between EtOAc (30 mL) and H₂O (8 mL). The organic phase was washed with 5% aq. NaHCO₃ (2×5 mL) and brine (5 mL), dried over Na₂SO₄, filtered, and concentrated to dryness in vacuo. The crude product was purified by silica gel flash chromatography (eluent A: heptane/DIEA (98:2); eluent B: EtOAc/DIEA (98:2)) to afford F18-4 (78 mg, 0.325 mmol, 54.7% yield) as a beige solid. Analytical method 10; t_{R}=0.68 min; [M+H]+=241.3.

### Example 11.22: Synthesis of (E)-4-(1-methyl-2-(pent-1-en-1-yl)-1H-imidazol-5-yl)phenol (F19-2)

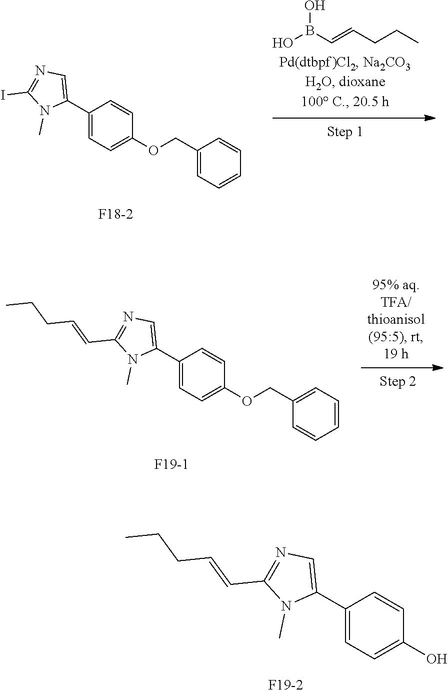

### Step 1. (E)-5-(4-(Benzyloxy)phenyl)-1-methyl-2-(pent-1-en-1-yl)-1H-imidazole (F19-1)

To F18-2 (234 mg, 600 μmol) was added (E)-pent-1-en-1-ylboronic acid (89 mg, 780 μmol), [1,1′-bis(di-tert-butylphosphino)ferrocene]dichloropalladium(II) (19.6 mg, 30 μmol), 1 M aq. Na₂CO₃ (1.800 mL, 1800 μmol) and dioxane (6 mL). Without stirring, the mixture was degassed and flushed with N₂ gas (5 times). The reaction mixture was stirred for 2 h at 100° C., and then partitioned between EtOAc (70 mL) and H₂O (15 mL). The organic phase was washed with 5% aq. NaHCO₃ (2×10 mL) and brine (10 mL), dried over Na₂SO₄, filtered, and concentrated to dryness in vacuo to give F19-1 (assumed to be 600 μmol). The crude product was used in the next step without purification. Analytical method 10; t_{R}=0.97 min; [M+H]⁺=333.3.

### Step 2. (E)-4-(1-Methyl-2-(pent-1-en-1-yl)-1H-imidazol-5-yl)phenol (F19-2)

F19-1 (600 μmol) was dissolved in 95% aq. TFA/thioanisol (95:5) (20 mL). The reaction mixture was stirred at rt for 19 h and then concentrated to dryness in vacuo. Toluene (20 mL) was added to the residue and the mixture was concentrated again to dryness in vacuo. The crude product was purified by normal phase flash chromatography using a silica gel column and eluting with 0-100% MeOH in cyclohexane. Pure fractions were combined and concentrated to dryness to afford F19-2 (180.7 mg, 492 μmol, 82% yield, two steps). Analytical method 10; t_{R}=0.61 min; [M+H]⁺=243.1.

### Example 11.23: Synthesis of 4-(2-(3,6-dihydro-2H-pyran-4-yl)-1-methyl-1H-imidazol-5-yl)phenol (F20-2)

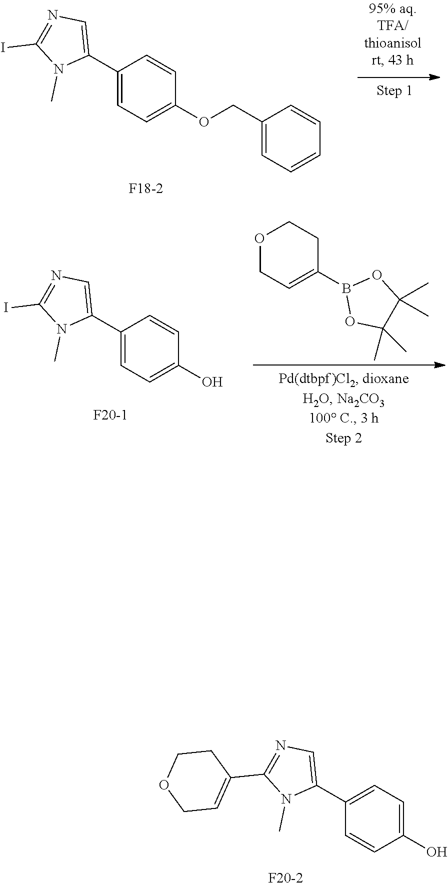

### Step 1. 4-(2-Iodo-1-methyl-1H-imidazol-5-yl)phenol (F20-1)

F18-2 (405.3 mg, 1.039 mmol) was dissolved in 95% aq. TFA/thioanisol (95:5) (20 mL) and the resulting mixture was stirred for 43 h at rt and then concentrated to dryness in vacuo. The obtained residue was partitioned between EtOAc (70 mL) and 5% aq. NaHCO₃ (10 mL). The organic phase was washed with 5% aq. NaHCO₃ (3×10 mL) and brine (10 mL), dried over Na₂SO₄, filtered, and concentrated to dryness in vacuo. The residue was purified by silica gel flash chromatography (eluent A: heptane/DIEA (98:2); eluent 1: EtOAc/DIEA; eluent B2: EtOAc/MeOH/DIEA (90:10:2)) to afford F20-1 (165 mg, 0.55 mmol, 53% yield). Analytical method 10; t_{R}=0.55 min; [M−H]⁻=299.0.

### Step 2. 4-(2-(3,6-Dihydro-2H-pyran-4-yl)-1-methyl-1H-imidazol-5-yl)phenol (F20-2)

To F20-1 (165 mg, 0.55 mmol), 2-(3,6-dihydro-2H-pyran-4-yl)-4,4,5,5-tetramethyl-1,3,2-dioxaborolane (139 mg, 0.660 mmol) and [1,1′-bis(di-tert-butylphosphino)ferrocene]dichloropalladium(II) (17.92 mg, 0.027 mmol) were added dioxane (10 mL) and 0.5 M aq. Na₂CO₃ (3.30 mL, 1.649 mmol). The resulting mixture was stirred for 3 h at 100° C. under a N₂ atmosphere and then partitioned between EtOAc (50 mL) and H₂O (10 mL). The organic phase was washed with 5% aq. NaHCO₃ (3×10 mL) and brine (10 mL), dried over Na₂SO₄, filtered, and concentrated to dryness in vacuo to afford F20-2 as a brown oil. The crude product was used in the next step without purification. Analytical method 10; t_{R}=0.42 min; [M+H]⁺=257.1.

### Example 11.24: Synthesis of tert-butyl 4-(5-(4-hydroxyphenyl)pyridin-3-yl)piperidine-1-carboxylate (F21-3)

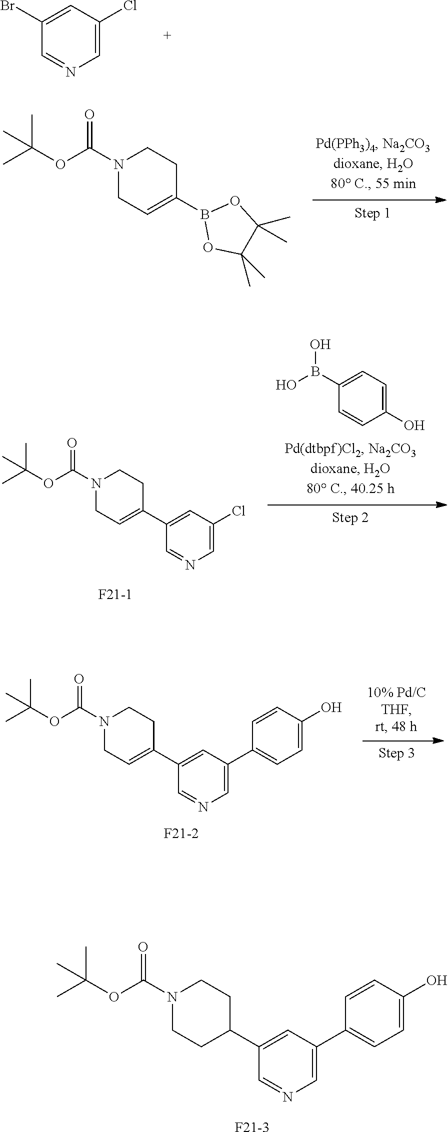

### Step 1. tert-Butyl 5-chloro-3′,6′-dihydro-[3,4′-bipyridine]-1′(2′H)-carboxylate (F21-1)

To a mixture of 3-bromo-5-chloropyridine (0.577 g, 3 mmol), tert-butyl 4-(4,4,5,5-tetramethyl-1,3,2-dioxaborolan-2-yl)-5,6-dihydropyridine-1(2H)-carboxylate (0.928 g, 3.00 mmol) and Pd(Ph₃P)₄ (0.173 g, 0.150 mmol) was added dioxane (9 mL) and 1 M aq. Na₂CO₃ (9.00 mL, 9.00 mmol). The reaction mixture was stirred for 55 min at 80° C. under an atmosphere of nitrogen and then cooled to rt and used in the next step without purification. Analytical method 10; t_{R}=1.17 min; [M+H]⁺=295.1.

### Step 2. tert-Butyl 5-(4-hydroxyphenyl)-3′,6′-dihydro-[3,4′-bipyridine]-1′(2′H)-carboxylate (F21-2)

To the solution of F21-1 from Step 1 was added 4-hydroxyphenyl)boronic acid (0.497 g, 3.60 mmol) and [1,1-bis(di-tert-butylphosphino)ferrocene]dichloropalladium(II) (0.098 g, 0.150 mmol). The resulting mixture was stirred for 16.25 h at 80° C. under an atmosphere of nitrogen and then cooled to rt. An additional portion of (4-hydroxyphenyl)boronic acid (0.497 g, 3.60 mmol) and [1,1-bis(di-tert-butylphosphino)ferrocene]dichloropalladium(II) (0.098 g, 0.150 mmol) was added and stirring was continued for 24 h at 80° C. under an atmosphere of nitrogen. The reaction mixture was partitioned between EtOAc (100 mL) and H₂O (20 mL). The organic phase was washed with 5% aq. NaHCO₃ (4×20 mL) and brine (20 mL), dried over Na₂SO₄, filtered, and concentrated to dryness in vacuo. The crude product was by normal phase flash chromatography, using a silica gel column and eluting with 0-100% EtOAc in heptane to afford F21-2 (467 mg, 1.325 mmol, 44% for 2 steps). Analytical method 10; t_{R}=0.99 min; [M+H]⁺=353.2.

### Step 3. tert-Butyl 4-(5-(4-hydroxyphenyl)pyridin-3-yl)piperidine-1-carboxylate (F21-3)

To F21-2 (467 mg, 1.325 mmol) dissolved in THF (15 mL) was added 10% Pd/C (141 mg, 0.133 mmol). The resulting suspension was stirred for 48 h at rt under an atmosphere of hydrogen and then filtered through Hyflo. The filtrate was concentrated to dryness in vacuo to afford F21-3 (assumed to be 1.325 mmol). The crude product was used in the next step without purification. Analytical method 10; t_{R}=0.94 min; [M+H]⁺=355.3.

### Example 11.25: Synthesis of 4-(1-methyl-2-(1-methylpyrrolidin-2-yl)-1H-imidazol-5-yl)phenol (F23-2)

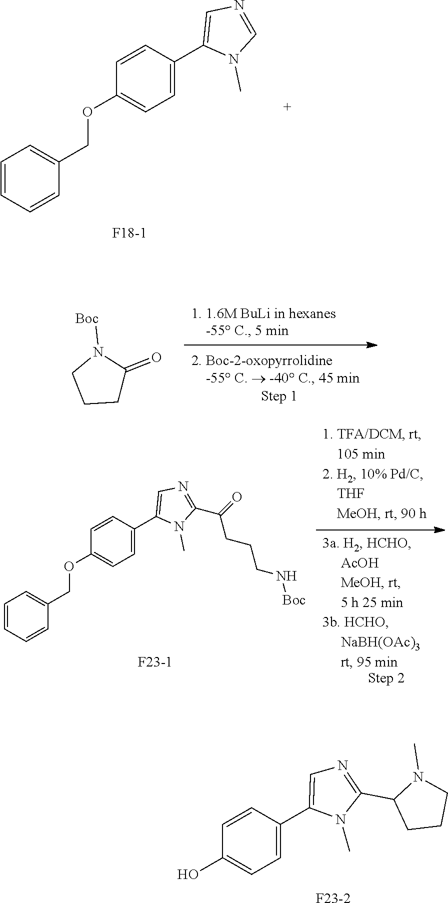

### Step 1. tert-Butyl (4-(5-(4-(benzyloxy)phenyl)-1-methyl-1H-imidazol-2-yl)-4-oxobutyl)carbamate (F-23-1)

To a solution of F18-1 (450 mg, 1.702 mmol) cooled to −55° C. was added 1.6 M n-BuLi in hexanes (1.702 mL, 2.72 mmol) dropwise. After stirring for 5 min at −55° C., tert-butyl 2-oxopyrrolidine-1-carboxylate (473 mg, 2.55 mmol) was added. The resulting mixture was stirred for 45 min and then allowed to warm to −40° C. The reaction mixture was quenched by the addition of AcOH (200 μL) and then concentrated in vacuo. The obtained residue was partitioned between EtOAc (50 mL) and 5% aq. NaHCO₃ (15 mL). The organic phase was washed with 5% aq. NaHCO₃ (2×10 mL) and brine (10 mL), dried over Na₂SO₄, filtered, and concentrated to dryness in vacuo to afford F23-1 (assumed to be 1.702 mmol) as a beige wax. The crude product was used in the next step without purification. Analytical method 10; t_{R}=1.27 min; [M+H]⁺=450.3.

### Step 2. 4-(1-Methyl-2-(1-methylpyrrolidin-2-yl)-1H-imidazol-5-yl)phenol (F23-2)

Step 2-1: F23-1 (967 mg, 1.70 mmol) was dissolved in TFA/DCM (1:1) (20 mL). The resulting solution was stirred for 105 min at rt and then concentrated to dryness in vacuo. The obtained residue was dissolved in toluene (5 mL) and the solution was concentrated to dryness in vacuo. The treatment with toluene was repeated.

Step 2-2: To a solution of the residue from Step 2-1 in THF/MeOH (1:1) (10 mL) was added 10% Pd/C (90 mg, 0.085 mmol). The suspension was stirred for 90 h at rt under an atmosphere of hydrogen.

Step 2-3: To the suspension from Step 2-2 was added 37% HCHO (0.253 mL, 3.40 mmol), AcOH (0.097 mL, 1.700 mmol), and MeOH (5 mL) and the resulting mixture was stirred under an atmosphere of hydrogen at rt for 5 h 25 min. An additional amount of 37% HCHO (0.253 mL, 3.40 mmol) and NaBH(OAc)₃ (721 mg, 3.40 mmol) were added and the mixture was stirred for 70 min. Additional NaBH(OAc)₃ (144 mg, 0.680 mmol) was added and stirring was continued for 25 min. The reaction mixture was filtered over Hyflo and the filtrate was concentrated to dryness in vacuo. The crude product was purified by silica gel flash chromatography (eluent A: EtOAc/MeOH/DIEA (95:5:2), eluent B: EtOAc/MeOH/DIEA (90:10:2)) to yield F23-2 (412 mg, 1.601 mmol, 94% yield for 2 steps) as a beige oil. Analytical method 10; t_{R}=0.44 min; [M+H]⁺=258.1.

### Example 11.26: Synthesis of 3,5-difluoro-4-(1-methyl-2-(pyrrolidin-1-ylmethyl)-1H-imidazol-5-yl)phenol (F24-3)

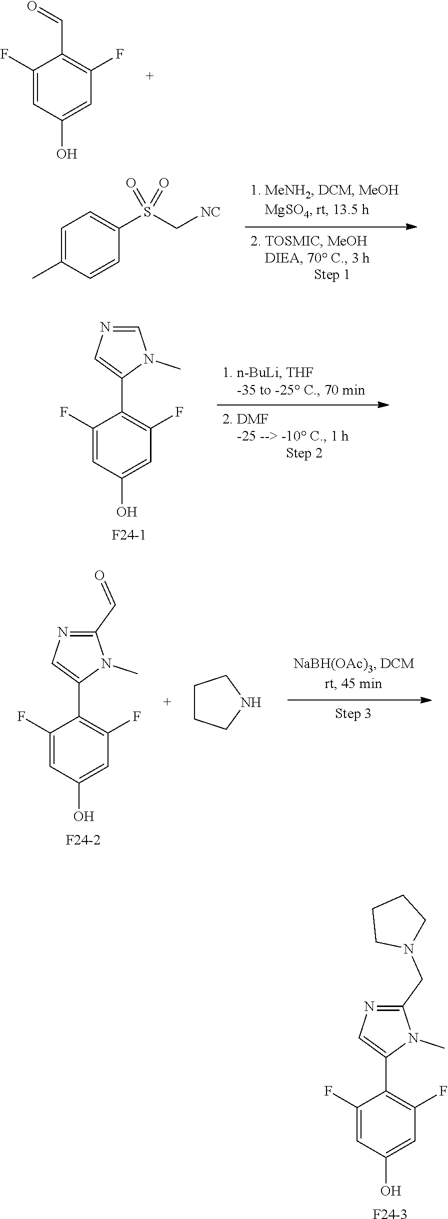

### Step 1. 3,5-Difluoro-4-(1-methyl-1H-imidazol-5-yl)phenol (F24-1)

Step 1-1: To 2,6-Difluoro-4-hydroxybenzaldehyde (949 mg, 6.00 mmol) dissolved in DCM (20 mL) was added 2 M methylamine in methanol (9.00 mL, 18.00 mmol), followed by MgSO₄ (4333 mg, 36.0 mmol). The resulting suspension was stirred for 13.5 h at rt and then filtered. The obtained residue was washed with DCM (˜40 mL) and the filtrate was concentrated to dryness in vacuo to afford an orange-brown solid.

Step 1-2: To TOSMIC (1523 mg, 7.80 mmol) was added a solution of the residue in MeOH (15 mL) and DIEA (3.14 mL, 18.00 mmol). The solution was stirred for 3 h at 70° C., then concentrated to dryness in vacuo. The crude product was purified by flash chromatography over silica gel (eluent A: EtOAc/MeOH/DIEA (95:5:2), eluent B: EtOAc/MeOH/DIEA (90:10:2)) to yield F24-1 (960 mg, 4.57 mmol, 76% yield) as a beige solid. Analytical method 11; t_{R}=0.71 min; [M+H]⁺=211.1.

### Step 2. 5-(2,6-Difluoro-4-hydroxyphenyl)-1-methyl-1H-imidazole-2-carbaldehyde (F24-2)

F24-1 (0.420 g, 2.00 mmol) was dissolved in THF (5.0 mL) and TTPA (5.0 mL) (Tris(N,N-tetramethylene)phosphoric acid triamide). The resulting solution was cooled to −30° C. and n-BuLi (1.6 M in hexanes) (7.50 mL, 12.00 mmol) was added dropwise. The reaction mixture was stirred for 65 min at −25 to −35° C. (some gel formed upon addition of n-BuLi). Additional TTPA (1.0 mL) and n-BuLi (1.6 M in hexanes) (1.25 mL, 2.00 mmol) were added and stirring at −25° C. was continued for 5 min. DMF (1.55 mL, 20.0 mmol) was added dropwise. The resulting mixture was stirred for 1 h, then allowed to warm to −10° C. and quenched by the addition of AcOH (0.916 mL, 16.0 mmol). The reaction mixture was partitioned between EtOAc (50 mL) and 5% aq. NaHCO₃ (15 mL). The organic phase was washed with 5% aq. NaHCO₃ (2×10 mL) and brine (10 mL), dried over Na₂SO₄, filtered, and concentrated to dryness in vacuo to afford F24-2 (assumed to be 2.0 mmol) as a yellow oil. The crude product was used in the next step without purification. Analytical method 11; t_{R}=1.10 min; [M+H]⁺=239.1.

### Step 3. 3,5-Difluoro-4-(1-methyl-2-(pyrrolidin-1-ylmethyl)-1H-imidazol-5-yl)phenol (F24-3)

F24-2 (2.0 mmol) was dissolved in DCM (20 mL) and pyrrolidine (0.331 mL, 4.00 mmol). The resulting solution was stirred for 5 min at rt and then NaBH(OAc)₃ (0.848 g, 4.00 mmol) was added. The reaction mixture was stirred for 45 min at rt and then partitioned between EtOAc (40 mL) and 1 M aq. HCl (10 mL). The organic phase was washed with 1 M aq. HCl (3×10 mL). The combined aqueous layers were basified to a pH of 8-9 by the addition of 4 M aq. NaOH and then extracted with EtOAc (3×40 mL). The combined organic phases were washed with brine (20 mL), dried over Na₂SO₄, filtered, and concentrated to dryness in vacuo to afford F24-3 (assumed to be 2.0 mmol) as a yellow oil. The crude product was used in the next step without purification. Analytical method 11; t_{R}=0.84 min; [M+H]⁺=294.2.

### Example 11.27: Synthesis of 4-(1-(2-fluoroethyl)-2-(pyrrolidin-1-ylmethyl)-1H-imidazol-5-yl)phenol (F25-3) and 4-(1-ethyl-2-(pyrrolidin-1-ylmethyl)-1H-imidazol-5-yl)phenol (F26-2)

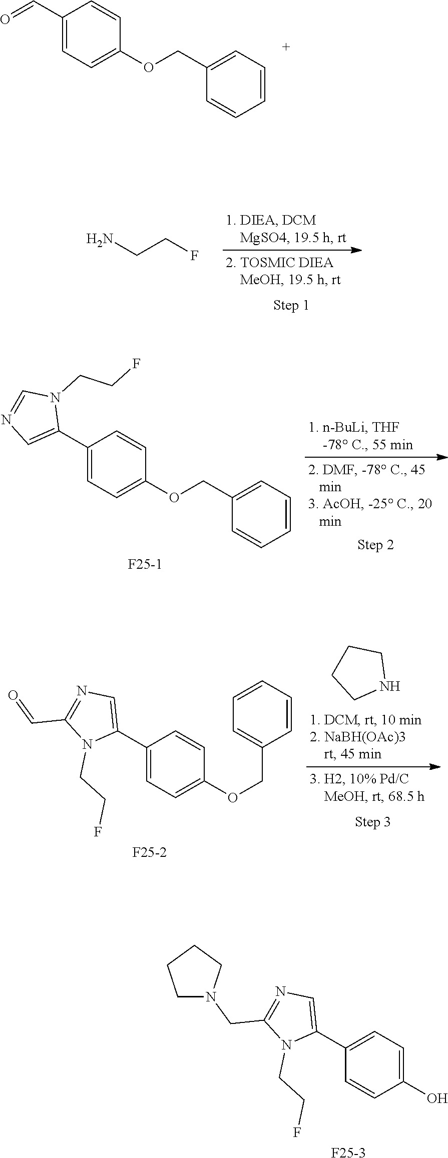

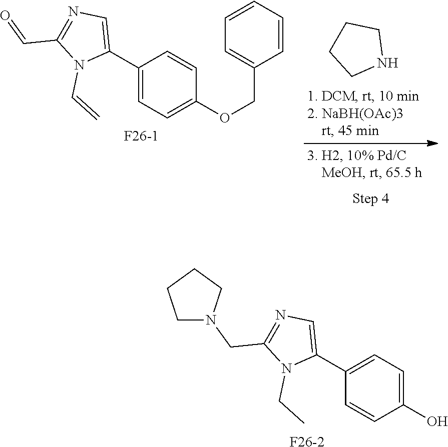

### Step 1. 5-(4-(Benzyloxy)phenyl)-1-(2-fluoroethyl)-1H-imidazole (F25-1)

To 4-(benzyloxy)benzaldehyde (1.061 g, 5 mmol), 2-fluoroethanamine (0.547 g, 5.50 mmol) and DIEA (1.223 mL, 7.00 mmol) in DCM (25 mL) was added MgSO₄ (3.61 g, 30.0 mmol). The resulting suspension was stirred for 19.5 h at rt, then filtered, and concentrated. The obtained residue was washed with DCM (5×10 mL). The combined filtrate and washing solutions were concentrated to dryness in vacuo. The residue was partitioned between DCM (70 mL) and H₂O (10 mL). The organic phase was washed with H₂O (5 mL) and brine (5 mL), dried over Na₂SO₄, filtered, and concentrated to dryness in vacuo. The residue was dissolved in MeOH (15 mL). TOSMIC (1.269 g, 6.50 mmol) and DIEA (2.62 mL, 15.00 mmol) were added, the reaction mixture was stirred for 19.5 h at rt and then concentrated to dryness in vacuo. The crude product was purified by silica gel flash chromatography (eluent A: EtOAc/DIEA (98:2), eluent B: EtOAc/MeOH/DIEA (95:5:2)) to yield F25-1 (1.07 g, 3.61 mmol, 72% yield) as a yellow solid. Analytical method 10; t_{R}=0.78 min; [M−H]⁻=295.0.

### Step 2. 5-(4-(Benzyloxy)phenyl)-1-(2-fluoroethyl)-1H-imidazole-2-carbaldehyde (F25-2) and 5-(4-(benzyloxy)phenyl)-1-vinyl-1H-imidazole-2-carbaldehyde (F26-1)

To a solution of F25-1 (942 mg, 3.18 mmol) in THF (30 mL) under an atmosphere of nitrogen at −78° C. was added BuLi (3.97 mL, 6.36 mmol) dropwise over 15 min (formation of a slightly cloudy red solution) and the resulting mixture was stirred for 55 min at −78° C. DMF (0.985 mL, 12.72 mmol) was then added dropwise stirring was continued for 45 min at −78° C. The reaction mixture was allowed to warm up to −25° C. and then acetic acid (0.728 mL, 12.72 mmol) was added dropwise. The reaction mixture was allowed to warm up to rt and then concentrated to dryness in vacuo. The obtained residue was partitioned between EtOAc (50 mL) and 5% aq. NaHCO₃ (25 mL). The organic phase was washed with 5% aq. NaHCO₃ (2×10 mL) and brine (10 mL), dried over Na₂SO₄, filtered, and concentrated to dryness in vacuo. The crude product was purified by silica gel flash chromatography (eluent A: heptane, eluent B: EtOAc) to give F25-2 (430.8 mg, 1.328 mmol, 41.8% yield) as a yellow solid (Analytical method 10; t_{R}=1.09 min; [M+H]⁺=325.2) and F26-1 (275.2 mg, 0.904 mmol, 28.4% yield) as a white solid (Analytical method 10; t_{R}=1.11 min; [M+H]⁺=304.9).

### Step 3 4-(1-(2-Fluoroethyl)-2-(pyrrolidin-1-ylmethyl)-1H-imidazol-5-yl)phenol (F25-3)

Step 3-1: F25-2 (430 mg, 1.326 mmol) and pyrrolidine (0.218 mL, 2.65 mmol) were dissolved in DCM (10 mL) and the resulting mixture was stirred for 10 min at rt. NaBH(OAc)₃ (562 mg, 2.65 mmol) was then added and stirring was continued for 45 min at rt. The reaction mixture was concentrated to dryness in vacuo. The residue was partitioned between EtOAc (50 mL) and 5% aq. NaHCO₃ solution (10 mL). The organic phase was washed with 5% aq. NaHCO₃ solution (2×5 mL) and brine (5 mL), dried over Na₂SO₄, filtered, and concentrated to dryness in vacuo.

Step 3-2: To the residue from Step 3-1 dissolved in MeOH (10 mL) was added 10% Pd/C (70.8 mg, 0.067 mmol) and the reaction mixture was stirred for 3 h at rt under an atmosphere of hydrogen. An additional amount of 10% Pd/C (142 mg, 0.133 mmol) was added and stirring under an atmosphere of hydrogen was continued for 65.5 h at rt. The reaction mixture was filtered through Hyflo and the filtrate was concentrated to dryness to give F25-3 (assumed to be 1.326 mmol). The crude product was used in the next step without purification. Analytical method 10; t_{R}=0.48 min; [M+H]⁺=290.1.

### Step 4. 4-(1-Ethyl-2-(pyrrolidin-1-ylmethyl)-1H-imidazol-5-yl)phenol (F26-2)

F-26-2 was prepared according to the procedure used for F25-3 starting from F26-1 (275 mg, 0.904 mmol). F26-2 (assumed to be 0.879 mmol) was obtained as a pink oil and used in the next step without purification. Analytical method 10; t_{R}=0.44 min; [M+H]⁺=273.2.

### Example 11.28: Synthesis of (S)-3-((S)-2-((S)-2-((4-chloro-2-(4-(4-((dimethylamino)methyl)-5-methyl-1H-imidazol-1-yl)phenoxy)benzyl)amino)propanamido)-3-methoxy-N-methylpropanamido)-4-(4-chlorophenyl)butanoic acid (F59-7)

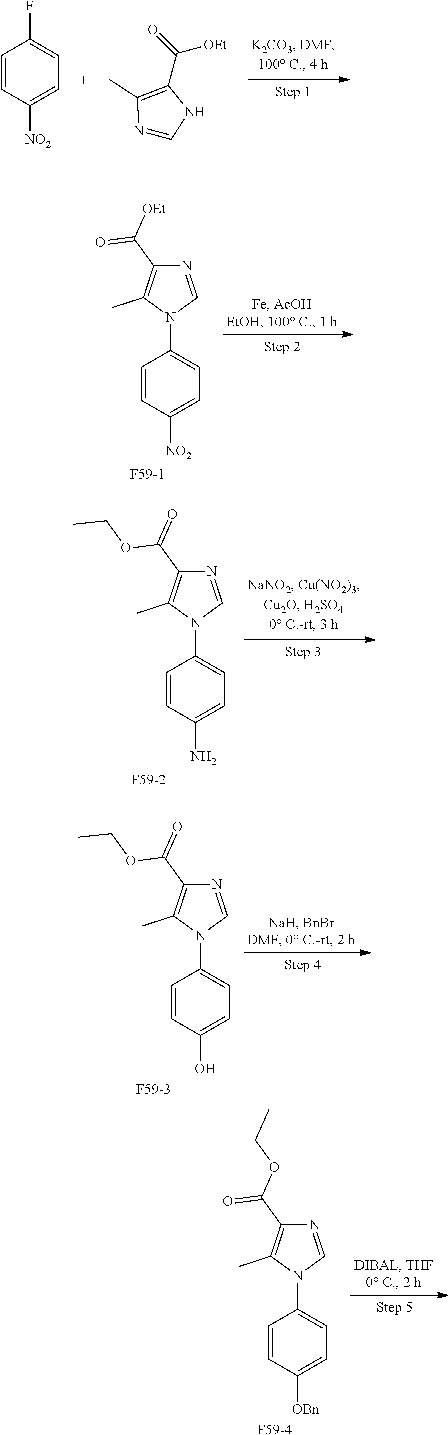

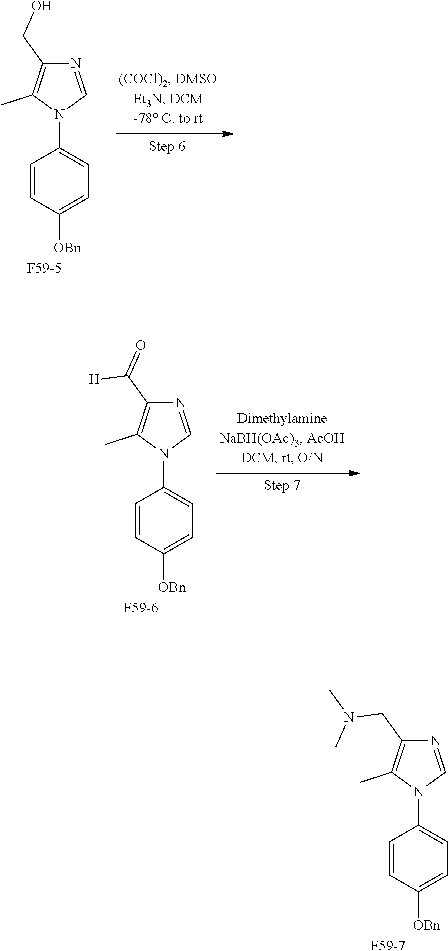

### Step 1. Ethyl 5-methyl-1-(4-nitrophenyl)-1H-imidazole-4-carboxylate (F59-1)

To a round bottom flask containing 1-fluoro-4-nitrobenzene (100 g, 0.71 mol) and ethyl 4-methyl-1H-imidazole-5-carboxylate (101 g, 0.71 mol) in DMF (800 mL) was added potassium carbonate (392 g, 2.84 mmol). The resulting mixture was heated to 100° C. and stirred for 4 h. The reaction mixture was then cooled to rt and poured into an ice bath to afford a slurry with stirring. The mixture was filtered and the filter cake was dried under high vacuum to afford F59-1 as a pale yellow solid (170 g, 87% yield). ¹H NMR (400 MHz, DMSO-d₆): δ 8.42 (d, J=6.8 Hz, 2H), 8.01 (s, 1H), 7.83 (d, J=6.8 Hz, 2H), 4.27 (q, J=7.2 Hz, 2H), 2.47 (s, 3H), 1.30 (t, J=7.2 Hz, 2H).

### Step 2. Ethyl 1-(4-aminophenyl)-5-methyl-1H-imidazole-4-carboxylate (F59-2)

To a mixture of F59-1 (85 g, 0.31 mol) in ethanol (700 mL) cooled in an ice bath was added acetic acid (200 mL), followed by iron powder (69 g, 1.24 mol). The ice bath was then removed and the mixture was heated to 100° C. and stirred for 1 h. The reaction mixture was warmed to room temperature, concentrated under reduced pressure and partitioned between DCM and water. The separated organic phase was dried over Na₂SO₄, filtered, and concentrated under reduced pressure to afford F59-2 as a gummy material (55 g, 73% yield). ¹H NMR (400 MHz, CDCl₃): δ 7.56 (s, 1H), 7.02 (d, J=8.0 Hz, 2H), 6.75 (d, J=8.4 Hz, 2H), 6.20 (bs, 2H), 4.38 (q, J=7.2 Hz, 2H), 2.41 (s, 3H), 1.41 (t, J=7.2 Hz, 2H).

### Step 3. Ethyl 1-(4-hydroxyphenyl)-5-methyl-1H-imidazole-4-carboxylate (F59-3)

To a mixture of F59-2 (25 g, 0.10 mol) in water (2 L) cooled in ice bath was added sulfuric acid (35%, 100 mL). The resulting mixture was stirred for 10 min and then NaNO₂ (14 g, 0.23 mol) was added with stirring while cold for another 10 min. Urea (6.13 g, 0.10 mol) was then added and the reaction mixture was then allowed to warm to rt. A solution of Cu(NO₃)₂ (370 g, 1.53 mol), followed by Cu₂O (7.3 g, 0.05 mol), were added. The resulting mixture was stirred at rt for 3 h, then quenched with aqueous ammonia, and repeatedly extracted with 10% methanol in DCM. The separated organic phase was dried over Na₂SO₄, filtered, and concentrated under reduced pressure to afford F59-3 as an off-white solid (16 g, 64% yield). The crude compound was used in next step without purification.

### Step 4. Ethyl 1-(4-(benzyloxy)phenyl)-5-methyl-1H-imidazole-4-carboxylate (F59-4)

To an ice cold suspension of NaH (2.85 g, 71.4 mmol) in DMF (75 mL) was added a solution of F59-3 (16 g, 65 mmol) in DMF (75 mL). The mixture was warmed to rt and stirred for 1 h. Benzyl bromide (13.3 g, 78 mmol) was added and stirring was continued at rt for ˜2 h. The reaction mixture was then quenched with ice-cold water and extracted twice with EtOAc. The separated organic phase was dried over Na₂SO₄, filtered, and concentrated under reduced pressure to afford a crude material. The crude product was purified by normal phase column chromatography using a silica gel column and eluting with with 0-40% EtOAc in ether to afford F59-4 as a pale yellow solid after drying down the pure fractions (8 g, 37% yield). ¹H NMR (400 MHz, CDCl₃): δ 7.50 (s, 1H), 7.48-7.36 (m, 5H), 7.18 (d, J=8.8 Hz, 2H), 7.08 (d, J=8.8 Hz, 2H), 5.12 (s, 2H), 4.39 (q, J=7.2 Hz, 2H), 2.43 (s, 3H), 1.42 (t, J=7.2 Hz, 2H).

### Step 5. (1-(4-(Benzyloxy)phenyl)-5-methyl-1H-imidazol-4-yl)methanol, (F59-5)

To a cooled solution of F59-4 (10 g, 30 mmol) in THF (150 mL) in an ice bath was added DIBAL-H (1 M in toluene, 98 mL, 98 mmol) and the resulting mixture stirred for 2. The reaction mixture was then quenched with water (21 mL) and then 5% aq NaOH (6 mL) was added. The resulting mixture was warmed to rt gradually and stirred for 15 min. The reaction mixture was filtered and the filtrate was concentrated under reduced pressure to afford F59-5 as an off-white solid after drying (8 g, 91% yield). ¹H NMR (400 MHz, DMSO-d₆): δ 7.59 (s, 1H), 7.48 (d, J=6.8 Hz, 2H), 7.41 (t, J=7.2 Hz, 2H), 7.36-7.30 (m, 3H), 7.14 (dd, J₁=2.0 Hz, J₂=6.8 Hz, 2H), 5.17 (s, 2H), 4.70 (t, J=5.6 Hz, 1H), 4.34 (d, J=5.6 Hz, 1H), 2.09 (s, 3H).

### Step 6. 1-(4-(Benzyloxy)phenyl)-5-methyl-1H-imidazole-4-carbaldehyde (F59-6)

To a pre-chilled solution of oxalyl chloride (4.7 mL, 54 mmol) in CH₂Cl₂ (80 mL) at −78° C. was added DMSO (7.7 mL, 108 mmol) dropwise. The resulting mixture was stirred at −78° C. for 10 min and a solution of F59-5 (8 g, 27 mmol) in CH₂Cl₂ (60 mL) was added dropwise. The mixture was stirred at −78° C. for 10 min and then Et₃N (23 mL, 162 mmol) was added dropwise. The resulting mixture was allowed to warm to rt and stirred for ˜3 h. The reaction mixture was diluted with water and extracted with DCM twice. The separated organic phase was dried over Na₂SO₄, filtered, and concentrated under reduced pressure to afford a crude product. The crude material was purified using a Grace prep HPLC system to afford F59-6 as an off-white solid after drying down the pure fractions (4 g, 50%). ¹H NMR (400 MHz, CDCl₃): δ 9.88 (s, 1H), 7.92 (s, 1H), 7.49-7.35 (m, 7H), 7.19 (d, J=6.8 Hz, 2H), 5.19 (s, 2H), 2.39 (s, 3H).

### Step 7. 1-(1-(4-(Benzyloxy)phenyl)-5-methyl-1H-imidazol-4-yl)-N,N-dimethylmethanamine (F59-7)

To a round bottom flask containing F59-6 (1.4 g, 4.79 mmol) in THF (42 mL) was added a solution of dimethylamine (2 M in THF, 4.79 mL, 9.58 mmol) and acetic acid (0.82 mL, 14.4 mmol) at rt. The resulting mixture was stirred for 1 h and then NaBH(OAc)₃ (2.2 g, 10.54 mmol) was added. The reaction mixture was stirred at rt overnight and then quenched with MeOH. The mixture was stirred until all gas evolution ceased and then concentrated to afford a crude material. The crude product was taken up in EtOAc and washed with a saturated solution of sodium bicarbonate. The organic layer was collected and a small amount of brine was added. The aqueous portion was back extracted with EtOAc once more. The combined organic phases were washed with brine (2×), dried over sodium sulfate, filtered, and concentrated to afford F59-7 as a yellow oil after drying under high vacuum (1.45 g, 94% yield). Analytical method 5; t_{R}=1.03 min; [M+H]⁺=322.1. The material was used in the next step without purification.

F60-1 and F61-1 in Table 30 below were prepared according to the procedure used for F59-7 (Example 11.28) starting from F59-6.

TABLE 30

| BB | | | |
|---|---|---|---|
| No. | Structure | Name | LCMS |
| F60-1 | 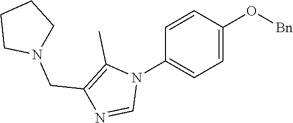 [SMILES: Cc1c(CN2CCCC2)ncn1-c1ccc(OCc2ccccc2)cc1] | 1-(4-(benzyloxy)phenyl)- 5-methyl-4-(pyrrolidin-1- ylmethyl)-1H-imidazole | Analytical method 5 t_{R} = 1.08 min [M + H]⁺ = 347.9 |
| F61-1 | 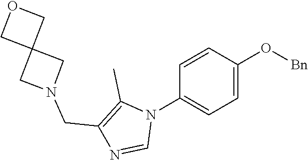 [SMILES: Cc1c(CN2CC3(COC3)C2)ncn1-c1ccc(OCc2ccccc2)cc1] | 6-((1-(4- (benzyloxy)phenyl)-5- methyl-1H-imidazol-4- yl)methyl)-2-oxa-6- azaspiro[3.3]heptane | Analytical method 5 t_{R} = 0.94 min [M + H]⁺ = 375.9 |

### Example 11.29: Synthesis of 1-(4-(benzyloxy)phenyl)-N-isopropyl-5-methyl-1H-imidazole-4-carboxamide (F56-2)

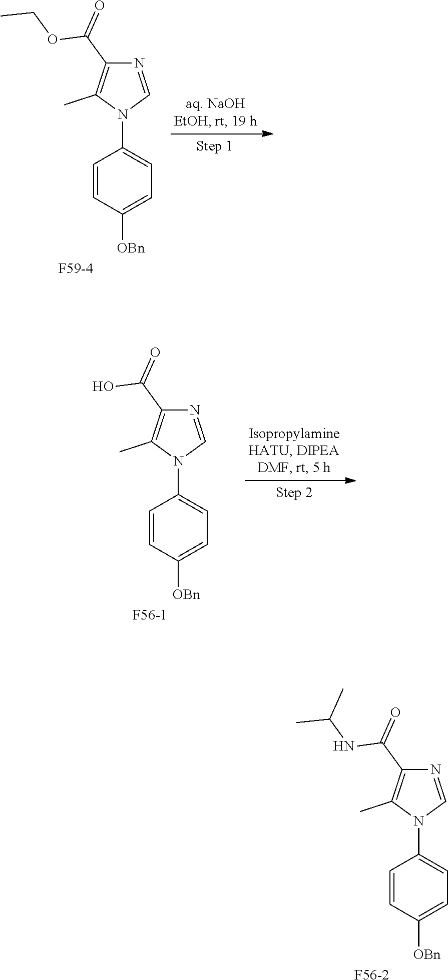

### Step 1. 1-(4-(Benzyloxy)phenyl)-5-methyl-1H-imidazole-4-carboxylic acid (F56-1)

Intermediate F59-4 (300 mg, 0.892 mmol) was suspended in EtOH (5 mL) and 1 M aq. NaOH (9 mL, 9 mmol) was added. The resulting mixture was allowed to stir at rt for 2 h and then concentrated to remove the EtOH. The pH of the remaining solution was adjusted to pH 5 using 1 M HCl. A precipitate formed which was collected and then further dried by lyophilization from MeCN/water. The precipitate was shown to be the product based on NMR analysis. F56-1 (264 mg, 96%) was obtained as a white solid. Analytical method 1; t_{R}=1.07 min; [M+H]⁺=309. ¹H NMR (400 MHz, DMSO-d₆) δ 7.76 (s, 1H), 7.52-7.31 (m, 7H), 7.18 (d, J=8.9 Hz, 2H), 5.19 (s, 2H), 2.35 (s, 3H).

### Step 2. 1-(4-(Benzyloxy)phenyl)-N-isopropyl-5-methyl-1H-imidazole-4-carboxamide (F56-2)

To F56-1 (50 mg, 0.162 mmol) suspended in DMF (0.8 mL) was added DIEA (0.140 mL, 0.802 mmol) and HATU (62 mg, 0.162 mmol). The resulting mixture was allowed to stir at rt for 5 min and isopropylamine (0.090 mL, 1.05 mmol) was added. The reaction mixture was allowed to stir at rt for 5 h, then partitioned between DCM and 10% aq. LiCl solution, and transferred to a separatory funnel. The phases were separated. The organic phase was washed with 10% aq. LiCl solution (2×), brine (1×), dried with sodium sulfate, and concentrated to provide F56-2 (50 mg, 88%) as a yellow solid, which was used in the next step without purification. Analytical method 1; t_{R}=1.39 min; [M+H]⁺=350.

F57-1 in Table 31 below was prepared according to the procedure use for F56-2 (example 11.29) starting from F56-1 and 2-aminoethan-1-ol.

TABLE 31

| BB | | | |
|---|---|---|---|
| No. | Structure | Name | LCMS |
| F57-1 | 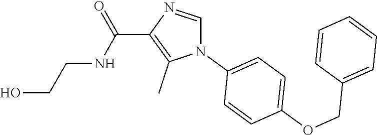 [SMILES: Cc1c(C(=O)NCCO)ncn1-c1ccc(OCc2ccccc2)cc1] | 1-(4-(benzyloxy)phenyl)- N-(2-hydroxyethyl)-5- methyl-1H-imidazole-4- carboxamide | Analytical method 1 t_{R} = 1.18 min [M + H]⁺ = 352.1 |

### Example 11.30: Synthesis of 4-(5-methyl-4-(morpholinomethyl)-1H-imidazol-1-yl)phenol (F62-5)

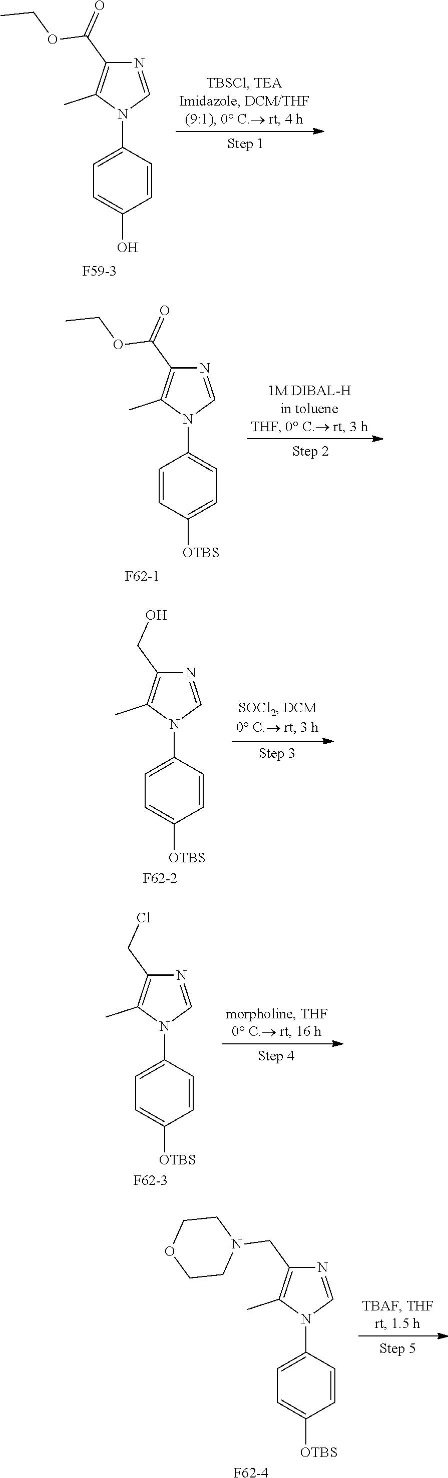

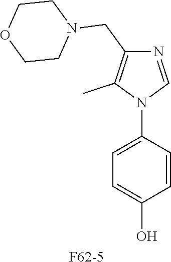 [SMILES: Cc1c(CN2CCOCC2)ncn1-c1ccc(O)cc1 ; "F62-5"]

### Step 1. Ethyl 1-(4-((tert-butyldimethylsilyl)oxy)phenyl)-5-methyl-1H-imidazole-4-carboxylate (F62-1)

To a stirred solution of F59-3 (3.5 g, 14.22 mmol) in CH₂Cl₂ (180 mL) and THF (20 mL) (9:1) under a nitrogen atmosphere, was added triethylamine (3.94 mL, 28.44 mmol), imidazole (0.096 g, 1.422 mmol) and tert-butylchlorodimethylsilane (4.28 g, 28.44 mmol) at 0° C. The resulting solution was allowed to stir at room temperature for 4 h and then evaporated to dryness. The obtained residue was dissolved in EtOAc (150 mL), washed with water (2×200 mL), dried over anhydrous Na₂SO₄ and concentrated under reduced pressure. The crude compound was purified by normal phase chromatography using a 40 g silica gel column eluting with 40-45% of EtOAc in Hexane to get the desired product F62-1 as an off white solid (4 g, 78%). ¹H NMR (400 MHz, CDCl₃): δ 7.47 (s, 1H), 7.13 (d, J=8.8 Hz, 2H), 6.95 (d, J=8.3 Hz, 2H), 4.43-4.37 (q, J=6.80 Hz, 2H), 2.44 (s, 3H), 1.42 (t, J=7.1 Hz, 3H), 1.01 (s, 9H), 0.25 (s, 6H).

### Step 2. (1-(4-((tert-Butyldimethylsilyl)oxy)phenyl)-5-methyl-1H-imidazol-4-yl)methanol (F62-2)

To a stirred solution of compound F62-1 (2.5 g, 6.94 mmol) in THF (50 mL) at 0° C. under nitrogen atmosphere was added 1.0 M solution of DIBAL-H in toluene (34.70 mL, 34.70 mmol). The resulting reaction mixture was allowed to stir at room temperature for 3 h. The reaction mixture was poured into ice cold aqueous sodium potassium tartrate solution (50 mL) carefully and extracted with EtOAc (2×200 mL). The combined organic phases were filtered through Celite®, dried over anhydrous Na₂SO₄ and concentrated under reduced pressure to get the desired product F62-2 as an off white solid (1.5 g, 68%). ¹H NMR (400 MHz, CDCl₃): δ 7.51 (s, 1H), 7.12 (d, J=8.8 Hz, 2H), 6.92 (d, J=8.3 Hz, 2H), 4.63 (s, 2H), 2.14 (s, 3H), 1.01 (s, 9H), 0.24 (s, 6H); LC-MS: t_{R}=1.00 min. (97.40%); 319.2 (M+1)

### Step 3. 1-(4-((tert-Butyldimethylsilyl)oxy)phenyl)-4-(chloromethyl)-5-methyl-1H-imidazole (F62-3)

To a stirred solution of F62-2 (0.30 g, 0.94 mmol) in DCM (20 mL) under CaCl₂ guard tube was added thionyl chloride (0.21 mL, 2.83 mmol) at 0° C. The resulting mixture was allowed to stir at room temperature r 3 h. After complete consumption of starting material, the reaction mixture was concentrated under reduced pressure and co-distilled with toluene twice under high vacuum to get the desired product F62-3 as a yellow sticky solid (0.30 g, crude). ¹H NMR (400 MHz, DMSO-d₆): δ 8.95 (s, 1H), 7.48 (d, J=8.8 Hz, 2H), 7.06 (d, J=8.8 Hz, 2H), 4.92 (s, 2H), 2.19 (s, 3H), 0.98 (s, 9H), 0.25 (s, 6H).

### Step 4. 4-((1-(4-((tert-Butyldimethylsilyl)oxy)phenyl)-5-methyl-1H-imidazol-4-yl)methyl)morpholine (F62-4)

To a stirred solution of F62-3 (0.30 g, 0.89 mmol) in dry THF (10 mL) at 0° C. under nitrogen atmosphere was added morpholine (0.38 mL, 4.46 mmol). The resulting mixture was allowed to stir at room temperature for 16 h. The reaction mixture was diluted with water (20 mL) and extracted with EtOAc (2×30 mL). The combined organic phases were washed with brine (10 mL), dried over anhydrous Na₂SO₄, and concentrated under reduced pressure. The crude compound was purified by normal phase chromatography using a 12 g silica gel column eluting with 3-4% methanol in dichloromethane to get the desired product F62-4 as a pale brown liquid (0.3 g, 65%). ¹H NMR (400 MHz, DMSO-d₆): δ 7.61 (s, 1H), 7.29 (d, J=8.8 Hz, 2H), 6.97 (d, J=8.8 Hz, 2H), 3.55 (t, J=4.4 Hz, 4H), 3.36 (s, 2H), 2.49-2.41 (m, 4H), 2.08 (s, 3H), 0.97 (s, 9H), 0.23 (s, 6H); LC-MS: t_{R}=1.16 min. (99.89%).

### Step 5. 4-(5-Methyl-4-(morpholinomethyl)-1H-imidazol-1-yl)phenol (F62-5)

To a solution of F62-4 (290 mg, 0.748 mmol) in THF (7 mL) was added TBAF (1.122 mL, 1.122 mmol) dropwise at rt. The reaction mixture was stirred for 1.5 hr, after which LCMS showed the reaction was completed. The reaction mixture was concentrated down and diluted with DCM/H₂O. The organic phase was separated. The aqueous layer was extracted with DCM twice. The combined organic phases were dried over sodium sulfate and concentrated to give F62-5 (196.5 mg, 96%) as an off-white solid, which was used directly without purification. Analytical method 5: t_{R}=0.51 mins; MS [M+H]⁺=274.2.

### Example 11.31: Synthesis of 1-(4-((tert-butyldimethylsilyl)oxy)phenyl)-5-methyl-1H-imidazole-4-carbaldehyde (F80-1)

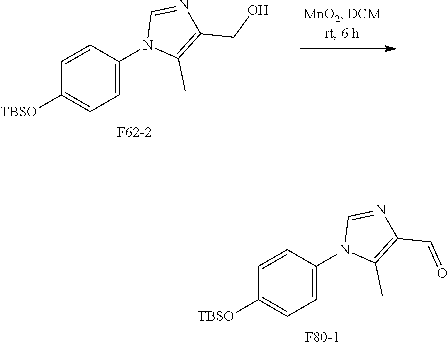

To a stirred solution of F62-2 (1.5 g, 4.71 mmol, 1.0 eq.) in DCM (30 mL) at rt, was added MnO₂ (4.09 g, 47.14 mmol, 10.0 eq.) under a nitrogen atmosphere. The resulting reaction mixture was stirred for 6 h at rt and then filtered through Celite® and washed with EtOAc. The filtrate was concentrated under reduced pressure to get the crude compound. The crude compound was purified by normal phase chromatography using a 12 g silica gel column eluting with 35-40% of EtOAc in hexane to provide F80-1 (1.2 g, 80% yield) as an off white solid. ¹H NMR (400 MHz, CDCl₃): δ 10.02 (s, 1H), 7.56 (s, 1H), 7.14 (d, J=8.8 Hz, 2H), 6.96 (d, J=8.8 Hz, 2H), 2.45 (s, 3H), 1.01 (s, 9H), 0.25 (s, 6H).

### Example 11.32: Synthesis of tert-butyl 4-(6-(5-chloro-2-formylphenoxy)-1-oxoisoindolin-2-yl)piperidine-1-carboxylate (F79)

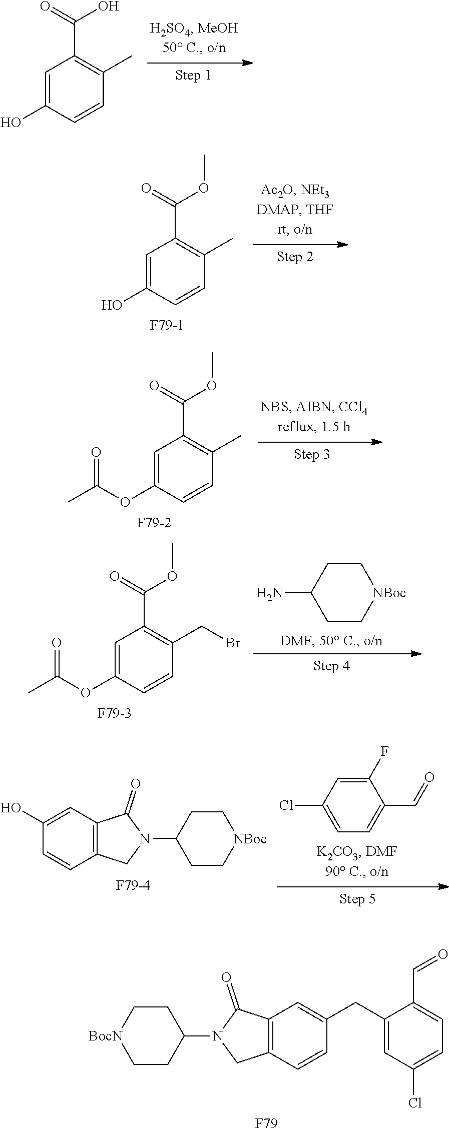

### Step 1. Methyl 5-hydroxy-2-methylbenzoate (F79-1)

To a solution of 5-hydroxy-2-methylbenzoic acid (1.91 g, 12.55 mmol) in methanol (31.3 mL) was added sulfuric acid (1 mL, 18.76 mmol). The resulting mixture was heated to 50° C. and stirred overnight. The reaction mixture was allowed to cool to rt and then poured into water (80 mL). The aqueous layer was extracted with EtOAc (3×80 mL), and the combined organic phases were washed with saturated sodium bicarbonate solution and brine, dried over sodium sulfate, filtered, and concentrated to provide F79-1 (2 g, 12.04 mmol, 96% yield). The crude residue was used directly in the following step without purification. Analytical method 7; t_{R}=1.05 min, [M+H]⁺=167.2. ¹H NMR (400 MHz, chloroform-d) b ppm 7.40 (d, J=2.78 Hz, 1H) 7.11 (d, J=8.21 Hz, 1H) 6.90 (dd, J=8.27, 2.84 Hz, 1H) 3.88 (s, 3H) 2.51 (s, 3H).

### Step 2. Methyl 5-acetoxy-2-methylbenzoate (F79-2)

To a solution of F79-1 (2 g, 12.04 mmol) in THF (48.1 ml) was added triethylamine (5.03 ml, 36.1 mmol), followed by DMAP (0.147 g, 1.204 mmol) and acetic anhydride (1.703 mL, 18.05 mmol). The resulting mixture was stirred at rt overnight. The reaction mixture was concentrated to remove the bulk of the THF, and the residue was diluted with EtOAc. The organic layer was washed with 0.1 N HCl, followed by water, saturated sodium bicarbonate solution, and brine, dried over sodium sulfate, filtered, and concentrated to provide F79-2. The crude residue was used directly in the following step without purification. Analytical method 7; t_{R}=1.28 min, [M+H]⁺=209.1. ¹H NMR (400 MHz, chloroform-d) b ppm 7.67 (d, J=2.65 Hz, 1H) 7.25 (s, 1H) 7.15 (dd, J=8.34, 2.53 Hz, 1H) 3.89 (s, 3H) 2.60 (s, 3H) 2.31 (s, 3H).

### Step 3. Methyl 5-acetoxy-2-(bromomethyl)benzoate (F79-3)

A solution of F79-2 (1.92 g, 9.22 mmol) in carbon tetrachloride (30.7 mL) was refluxed for 1.5 h in the presence of N-bromosuccinimide (1.805 g, 10.14 mmol) and AIBN (0.151 g, 0.922 mmol). After removal of the succinimide by filtration, the solvent was evaporated and the product was purified by silica gel column chromatography (eluting with 0-20% EtOAc in heptanes) to provide F79-3 (2.0 g, 6.97 mmol, 76% yield). Analytical method 7; t_{R}=1.35 min, [M+NH₄]⁺=304.2. Method 5; t_{R}=1.24 min, [M+NH₄]⁺=304.2.

### Step 4. tert-Butyl 4-(6-hydroxy-1-oxoisoindolin-2-yl)piperidine-1-carboxylate (F79-4)

To a solution of tert-butyl 4-aminopiperidine-1-carboxylate (4.46 g, 22.29 mmol) in DMF (27.9 mL) was added F79-3 (2.0 g, 6.97 mmol) and the resulting mixture was stirred at 50° C. overnight. The reaction mixture was poured into saturated aqueous ammonium chloride and EtOAc (50:50), and the pH of the aqueous phase was adjusted to neutral with 4 M HCl. The aqueous phase was extracted twice more with EtOAc, and the combined organic phases were washed with water (3×) and brine, dried over sodium sulfate, filtered, and concentrated. The residue was purified by normal phase chromatography using a silica gel column (eluting with 25-100% EtOAc in heptanes) to provide F79-4 (1.84 g, 5.54 mmol, 79% yield), as an off-white foam. Analytical method 7; t_{R}=1.19 min, [M+H]⁺=333.4. Method 5; t_{R}=1.06 min, [M+H]⁺=333.4.

### Step 5. tert-Butyl 4-(6-(5-chloro-2-formylphenoxy)-1-oxoisoindolin-2-yl)piperidine-1-carboxylate (F79)

To a mixture of 4-chloro-2-fluorobenzaldehyde (0.41 g, 2.59 mmol) and F79-4 (0.860 g, 2.59 mmol) in anhydrous DMF (10 mL) was added K₂CO₃ (0.715 g, 5.17 mmol) in one portion. The resulting mixture was stirred at 90° C. under an atmosphere of nitrogen overnight. The reaction mixture was cooled to rt and diluted with water and EtOAc. The organic phase was separated, washed with water, dried over sodium sulfate, filtered, and concentrated. The crude was purified by normal phase chromatography using a silica gel column (eluting with 0-70% EtOAc/heptane) to afford F79 (278 mg, 22.8%) as a white foam. Analytical method 7; t_{R}=1.27 min; [M-C(CH₃)₃+H]⁺=415.2. ¹H NMR (400 MHz, dichloromethane-d₂) b ppm 10.47 (d, J=0.86 Hz, 1H) 7.91 (d, J=8.44 Hz, 1H) 7.58 (d, J=8.19 Hz, 1H) 7.49 (d, J=2.20 Hz, 1H) 7.36 (dd, J=8.19, 2.32 Hz, 1H) 7.24 (ddd, J=8.41, 1.86, 0.73 Hz, 1H) 6.90 (d, J=1.83 Hz, 1H) 4.40 (s, 2H) 4.34-4.45 (m, 1H) 4.28 (dt, J=13.63, 2.11 Hz, 2H) 2.84-2.95 (m, 2H) 1.82-1.90 (m, 2H) 1.64-1.78 (m, 2H) 1.46-1.51 (s, 9H).

### Example 11.33: Synthesis of 2-(2-oxo-2-(5H-pyrrolo[3,4-b]pyridin-6(7H)-yl)ethoxy)benzaldehyde (F78)

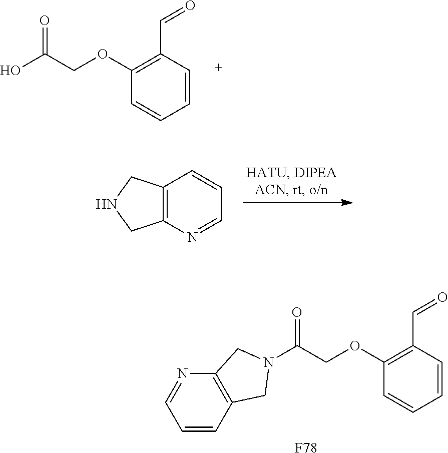 [SMILES: O=Cc1ccccc1OCC(=O)O ; c1cnc2c(c1)CNC2 ; O=Cc1ccccc1OCC(=O)N1Cc2cccnc2C1 ; "F78"]

To a vial containing 2-(2-formylphenoxy)acetic acid (300 mg, 1.67 mmol), 6,7-dihydro-5H-pyrrolo[3,4-b]pyridine (338 mg, 1.75 mmol) and HATU (633 mg, 1.67 mmol) was added ACN (8 mL). The resulting solution was stirred at rt and DIPEA (1.45 mL, 8.33 mmol) was then added. The reaction mixture was stirred overnight and then concentrated under reduced pressure. The obtained residue was taken up in DCM, washed with water twice, dried over sodium sulfate, filtered, and concentrated under reduced pressure. The crude material was via normal phase flash chromatography using a silica gel column and eluting with 0-10% MeOH in DCM to afford the desired product as a pale solid after concentrating the pure fractions (231 mg, 47% yield). Analytical method 1; t_{R}=0.89 min; [M+H]⁺=283.0.

### Example 11.34: Synthesis of 4-(difluoromethyl)-2-fluorobenzaldehyde (F46-1)

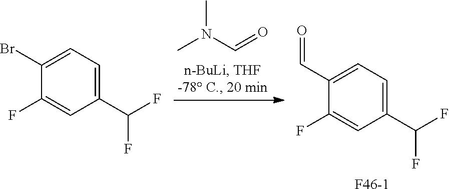

To a solution of 1-bromo-4-(difluoromethyl)-2-fluorobenzene (950 mg, 4.22 mmol) in THF (20 mL) at −78° C. under a N₂ atmosphere was added 1.6 M n-butyllithium in hexanes (2.77 mL, 4.43 mmol) over 5 min. The resulting mixture turned dark green and was stirred for 30 min at −78° C. DMF (0.490 mL, 6.33 mmol) was then added dropwise over 1 min and the reaction mixture was stirred for 20 min, then quenched with 1 M HCl/MeOH (2:1) (3 mL), and warmed to rt (orange solution). H₂O (5 mL) was added (pH ˜5) and the aqueous phase was extracted with diethyl ether (3×5 mL). The combined organic phases were washed with 1 M NaOH (10 mL) and brine (10 mL), dried over MgSO₄, filtered, and concentrated in vacuo to afford F46-1 as a dark orange oil (920 mg, 125%). The crude material was taken onto next step without purification. Analytical method 5; t_{R}=0.83 min; [M−H]⁻=173.2.

### Example 12: Building Block G—Benzylated Building Block E

### Example 12.1: Synthesis of (4-chloro-2-(4-(2-((dimethylamino)methyl)-1-methyl-1H-imidazol-5-yl)phenoxy)benzyl)-L-alanine (G1)

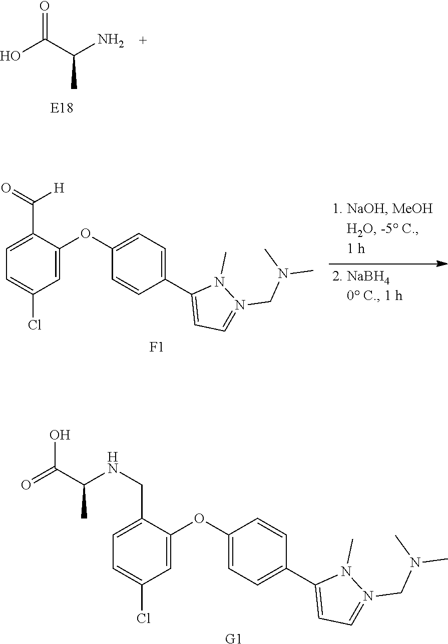

To a suspension of L-alanine (E18) (257 mg, 2.88 mmol) in MeOH (4 mL) and water (0.4 mL) at rt was added NaOH (115 mg, 2.88 mmol). The resulting mixture was allowed to stir for 30 min at rt. Intermediate F1 (1.02 g, 2.74 mmol) was then added and the reaction mixture was cooled to −5° C. and allowed to stir for 1 h. NaBH₄ (42 mg, 1.1 mmol) was added in portions maintaining the internal reaction temperature below 0° C. The reaction mixture was stirred at 0° C. for 1 h and then quenched by adding water dropwise until gas evolution stopped. The mixture was concentrated and the aqueous phase was extracted with EtOAc (2×50 mL). The aqueous phase was loaded onto a pad of dowex. The pad was eluted with water followed by 2 N NH₄OH in water. The aqueous filtrate was lyophilized to provide a white solid. The solid was then dissolved in water and then acidified to pH of ˜7. The aqueous phase was extracted with 20% isopropanol in CHCl₃ (100 mL) several times. Brine was added to the aqueous layer and extraction continued. The combined organic phases were dried over Na₂SO₄, filtered, and concentrated to yield white solid G1 (510 mg, 42%). Analytical method 7; t_{R}=0.49 min; [M+H]⁺=443.3.

The Building Blocks (BB) shown in Table 32 below were prepared according to the procedure used for G1 (Example 12.1) starting from the corresponding amino acids in Table 24.

TABLE 32
Building block G

| | | | Synthesis |
|---|---|---|---|
| BB | | | procedure/ |
| No. | Structure | Name | LCMS |
| G2 | 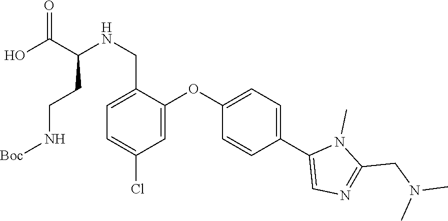 [SMILES: CN(C)Cc1ncc(-c2ccc(Oc3cc(Cl)ccc3CN[C@@H](CCNC(=O)OC(C)(C)C)C(=O)O)cc2)n1C] | (S)-4-((tert- butoxycarbonyl)amino)-2- ((4-chloro-2-(4-(2- ((dimethylamino)methyl)- 1-methyl-1H-imidazol-5- yl)phenoxy)benzyl)amino) butanoic acid | G1 (Example 12.1)/ Analytical method 7 t_{R} = 0.66 min [M + H]⁺ = 572.0 |
| G3 | 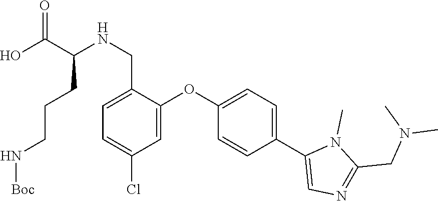 [SMILES: CN(C)Cc1ncc(-c2ccc(Oc3cc(Cl)ccc3CN[C@@H](CCCNC(=O)OC(C)(C)C)C(=O)O)cc2)n1C] | (S)-5-((tert- butoxycarbonyl)amino)-2- ((4-chloro-2-(4-(2- ((dimethylamino)methyl)- 1-methyl-1H-imidazol-5- yl)phenoxy)benzyl)amino) pentanoic acid | G1 (Example 12.1)/ Analytical method 7 t_{R} = 0.67 min [M + H]⁺ = 586.2 |
| G4 | 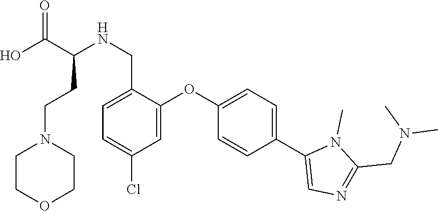 [SMILES: CN(C)Cc1ncc(-c2ccc(Oc3cc(Cl)ccc3CN[C@@H](CCN3CCOCC3)C(=O)O)cc2)n1C] | (S)-2-((4-chloro-2-(4-(2- ((dimethylamino)methyl)- 1-methyl-1H-imidazol-5- yl)phenoxy)benzyl)amino)- 4-morpholinobutanoic acid | G1 (Example 12.1)/ Analytical method 5 t_{R} = 0.60 min [M + H]⁺ = 542.2 |
| G5 | 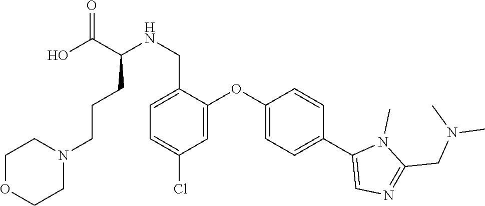 [SMILES: CN(C)Cc1ncc(-c2ccc(Oc3cc(Cl)ccc3CN[C@@H](CCCN3CCOCC3)C(=O)O)cc2)n1C] | (S)-2-((4-chloro-2-(4-(2- ((dimethylamino)methyl)- 1-methyl-1H-imidazol-5- yl)phenoxy)benzyl)amino)- 5-morpholinopentanoic acid | G1 (Example 12.1)/ Analytical method 5 t_{R} = 0.59 min [M + 1]⁺ = 556.4 |
| G6 | 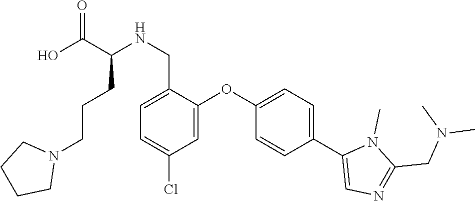 [SMILES: CN(C)Cc1ncc(-c2ccc(Oc3cc(Cl)ccc3CN[C@@H](CCCN3CCCC3)C(=O)O)cc2)n1C] | (S)-2-((4-chloro-2-(4-(2- ((dimethylamino)methyl)- 1-methyl-1H-imidazol-5- yl)phenoxy)benzyl)amino)- 5-(pyrrolidin-1- yl)pentanoic acid | G1 (Example 12.1)/ Analytical method 5 t_{R} = 0.72 min [M + H]⁺ = 540.3 |
| G7 | 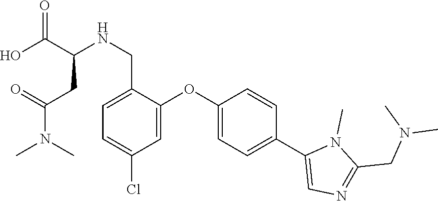 [SMILES: CN(C)Cc1ncc(-c2ccc(Oc3cc(Cl)ccc3CN[C@@H](CC(=O)N(C)C)C(=O)O)cc2)n1C] | (S)-2-((4-chloro-2-(4-(2- ((dimethylamino)methyl)- 1-methyl-1H-imidazol-5- yl)phenoxy)benzyl)amino)- 4-(dimethylamino)-4- oxobutanoic acid | G1 (Example 12.1)/ Analytical method 5 t_{R} = 0.57 min [M + H]⁺ = 513.7 |
| G8 | 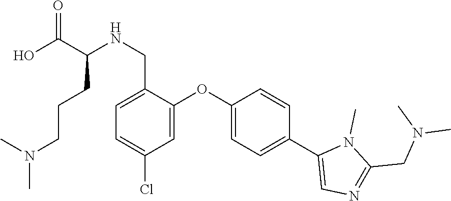 | (S)-2-((4-chloro-2-(4-(2- ((dimethylamino)methyl)- 1-methyl-1H-imidazol-5- yl)phenoxy)benzyl)amino)- 5-(dimethylamino)pentanoic acid | G1 (Example 12.1)/ Analytical method 5 t_{R} = 0.68 min [M + 1]⁺ = 514.0 |

### Example 12.2: Synthesis of N-(((9H-fluoren-9-yl)methoxy)carbonyl)-N-(2-(4-azidophenoxy)-4-chlorobenzyl)-L-alanine (G9)

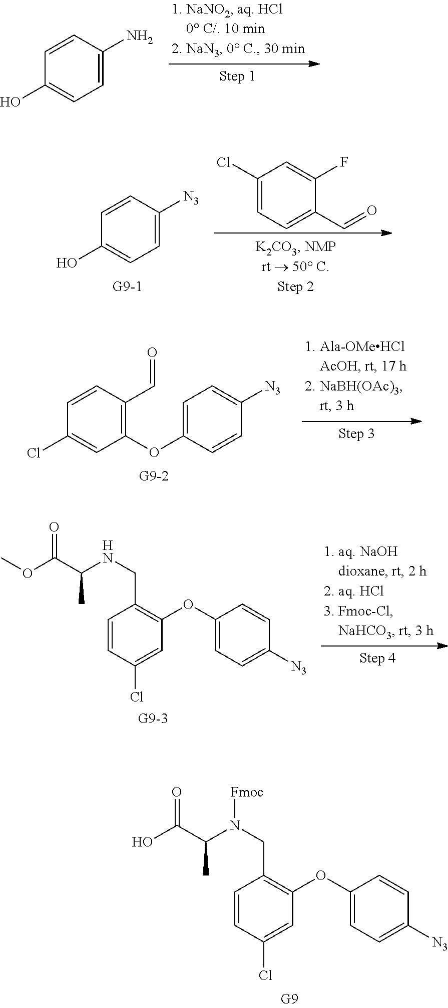

### Step 1. 4-Azidophenol (G9-1)

To 4-Aminophenol (1.00 g, 9.16 mmol) suspended in 2 N aq. HCl (18 mL) and cooled to 0° C. was added a solution of sodium nitrite (948 mg, 13.8 mmol) in water (1.5 mL) dropwise. The resulting mixture was allowed to stir for 10 min and a solution of sodium azide (953 mg, 14.7 mmol) in water was added dropwise (effervescence was observed). The reaction mixture was allowed to stir for 30 min and then partitioned between water and DCM The layers were separated in a separatory funnel. The aqueous phase was extracted with DCM (2×). The combined organic phases were dried over sodium sulfate, filtered, and concentrated to provide G9-1 (890 mg, 72%) as a brown oil, which was carried onto the next step without purification. ¹H NMR (400 MHz, CDCl₃) δ 6.94-6.89 (m, 2H), 6.85-6.80 (m, 2H).

### Step 2. 2-(4-Azidophenoxy)-4-chlorobenzaldehyde (G9-2)

G9-1 (890 mg, 6.59 mmol) and 2-(4-azidophenoxy)-4-chlorobenzaldehyde (1.04 g, 6.59 mmol) were suspended in NMP (16.5 mL) at rt. The resulting mixture was allowed to stir for 17 h and then heated to 50° C. for 23 h. The reaction mixture was partitioned between EtOAc and brine. The biphasic mixture was transferred to a separatory funnel. The layers were separated. The organic layer was washed with water (4×) and brine (1×), dried over sodium sulfate, filtered, and concentrated. Purification on silica gel (heptane/ethyl acetate) provided G9-2 (1.40 g, 78%) as a yellow oil. Analytical method 1; t_{R}=1.69 min; [M+H]⁺=273.9. ¹H NMR (400 MHz, CDCl₃) δ 10.49 (d, J=0.8 Hz, 1H), 7.90 (d, J=8.4 Hz, 1H), 7.17 (ddd, J=8.4, 1.9, 0.8 Hz, 1H), 7.12 (s, 4H), 6.82 (d, J=1.8 Hz, 1H).

### Step 3. Methyl (2-(4-azidophenoxy)-4-chlorobenzyl)-L-alaninate (G9-3)

To Intermediate G9-2 (1.40 g, 5.12 mmol) and Ala-OMe.HCl (1.07 g, 7.67 mmol) suspended in DCM (50 mL) at rt was added AcOH (0.590 mL, 10.3 mmol). The resulting mixture was allowed to stir at rt for 17 h and sodium triacetoxyborohydride (2.80 g, 13.2 mmol) was added in portions. Effervescence was observed. The reaction mixture was allowed to stir at rt for 3 h and then quenched with sat. sodium bicarbonate solution and DCM was added. The biphasic mixture was transferred to a separatory funnel and the phases were separated. The aqueous phase was extracted with DCM (2×). The combined organic phases were washed with sat. sodium bicarbonate solution and brine, dried over magnesium sulfate, filtered, and concentrated to provide G9-3 (1.72 g, 68%) as an orange oil in 73% purity by LCMS. The material was used in the next step without purification. Analytical method 1; t_{R}=1.11 min; [M+H]⁺=361.

### Step 4. N-(((9H-Fluoren-9-yl)methoxy)carbonyl)-N-(2-(4-azidophenoxy)-4-chlorobenzyl)-L-alanine (G9)

To intermediate G9-3 (1.72 g, 3.48 mmol) suspended in dioxane (40 mL) was aqueous 1 M NaOH (12 mL). The resulting mixture was allowed to stir at rt for 2 h and then the remaining base was quenched by the addition of 1 M aq. HCl (7 mL). Fmoc-Cl (1.36 g, 5.26 mmol), sodium carbonate (1.07 g, 10.1 mmol), and water (10 mL) were then added. The reaction mixture was allowed to stir at rt for 3 h, and then quenched by the addition of 1 M aq. HCl, and EtOAc was added. The biphasic mixture was transferred to a separatory funnel and the phases were separated. The aqueous phase was extracted with EtOAc (2×). The combined organic phases were dried over magnesium sulfate, filtered, and concentrated. Purification on silica gel (eluting with heptane/EtOAc with 0.1% AcOH) provided the product as an off-white foam. The foam was lyophilized from MeCN/water to remove residual solvent, yielding G9 (1.45 g, 51%) as a tan solid in 95% purity. Analytical method 7; t_{R}=1.60 min; [M+H]⁺=569.2.

### Example 12.3: Synthesis of (S)-2-((4-chloro-2-(4-(2-((dimethylamino)methyl)-1-methyl-1H-imidazol-5-yl)phenoxy)-benzyl)amino)-5-(3,3,4,4-tetrafluoropyrrolidin-1-yl)pentanoic acid (G10)

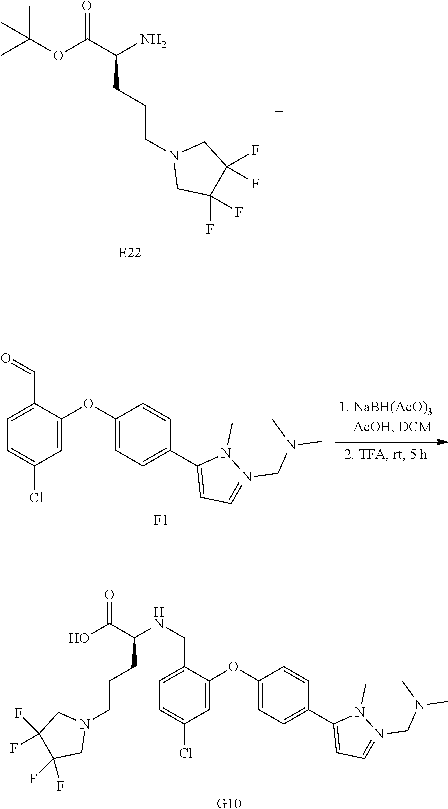

To a solution of (S)-tert-butyl 2-amino-5-(3,3,4,4-tetrafluoropyrrolidin-1-yl)pentanoate E22 (0.442 g, 1.405 mmol) and 4-chloro-2-(4-(2-((dimethylamino)methyl)-1-methyl-1H-imidazol-5-yl)phenoxy)benzaldehyde F1 (1.039 g, 2.81 mmol) in anhydrous DCM (10 mL) was added AcOH (0.322 mL, 5.62 mmol). The resulting mixture was stirred for 1 h before NaBH(AcO)₃ (1.489 g, 7.03 mmol) was added. The reaction mixture was then stirred for an additional 1.5 h, after which LCMS showed complete consumption of starting material. The reaction was quenched by the slow addition of NaHCO₃ sat. aq. solution (50 mL) at 0° C. and then EtOAc (100 mL). The organic phase was separated, washed with water (50 mL) and brine, dried over Na₂SO₄ and concentrated. The crude material was concentrated and purified by chromatography on a 80 g C18-column eluting with 0-100% MeCN/water, 0.1% NH₄OH.

Deprotection: TFA (10 mL) was added to the purified intermediate and the mixture was stirred at room temperature for 5 h. LCMS showed full conversion to desired product. The reaction mixture was concentrated, 10 mL of toluene were added and the mixture was concentration. ACN/water was added to the residue for lyophilization and the product was used in the next step without purification. Analytical method 5; t_{R}=0.67 min; [M+H]⁺=612.1.

### Example 12.4: Synthesis of N-(((9H-fluoren-9-yl)methoxy)carbonyl)-N-(4-chloro-2-(3-(methoxycarbonyl)phenoxy)-benzyl)-L-alanine (G11)

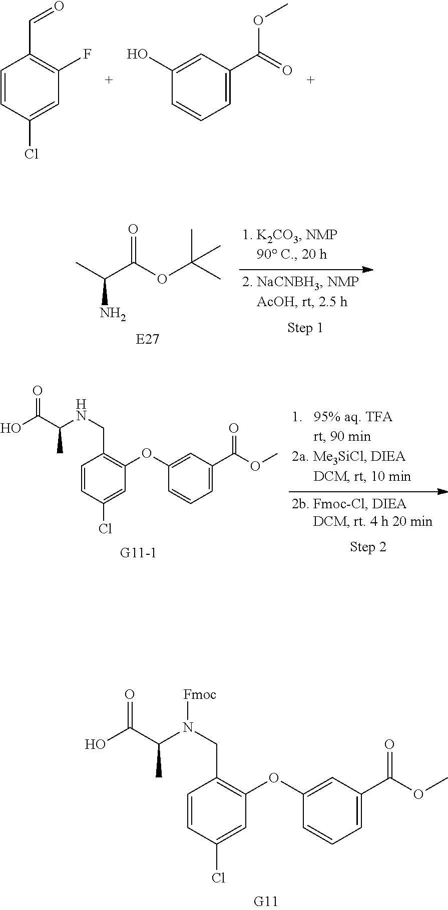

### Step 1. Methyl (S)-3-(2-(((1-(tert-butoxy)-1-oxopropan-2-yl)amino)methyl)-5-chlorophenoxy)benzoate (G11-1)

To 4-chloro-2-fluorobenzaldehyde (0.238 g, 1.50 mmol), methyl 3-hydroxybenzoate (0.228 g, 1.500 mmol) and K₂CO₃ (0.415 g, 3.00 mmol) was added NMP (4 mL). The resulting mixture was stirred for 20 h at 90° C. NMP (6 mL) and AcOH (0.429 mL, 7.50 mmol) were added and stirring was continued for 1 h at rt. H-Ala-OtBu HCl (E27, 0.409 g, 2.250 mmol) was added and the reaction mixture was stirred for another 2.5 h at rt. NaCNBH₃ (0.283 g, 4.50 mmol) was added and stirring was continued for 2.5 h at rt. The reaction mixture was partitioned between EtOAc (60 mL) and 5% aq. NaHCO₃ (15 mL). The organic phase was washed with 5% aq. NaHCO₃ (2×15 mL), half-saturated KH₂PO₄ (4×15 mL) and brine (10 mL), dried over Na₂SO₄, filtered, and concentrated to dryness in vacuo to afford G11-1 (assumed to be 1.50 mmol) as a brown oil. The crude product was used in the next step without purification. Analytical method 10; t_{R}=1.05 min; [M+H]⁺=420.6.

### Step 2: N-(((9H-fluoren-9-yl)methoxy)carbonyl)-N-(4-chloro-2-(3-(methoxycarbonyl)phenoxy)benzyl)-L-alanine (G11)

Step 2-1: G11-1 (1.500 mmol) was dissolved in 95% aq. TFA (10 mL). The solution was stirred for 90 min at rt and then concentrated to dryness in vacuo. The residue was dissolved in DCM and the solution was concentrated to dryness in vacuo.

Step 2-2: To the residue from Step 2-1 dissolved in DCM (15 mL) was added DIEA (1.310 mL, 7.50 mmol) and trimethylsilylchloride (0.575 mL, 4.50 mmol) and the resulting solution was stirred for 10 min at rt. A solution of Fmoc-Cl (0.349 g, 1.350 mmol) in DCM (5 mL) was added stirring was continued for 1 h at rt. Additional Fmoc-Cl (0.039 g, 0.150 mmol) in DCM (2 mL) was added and stirring was continued for 1 h 50 min. DIEA (0.262 mL, 1.500 mmol) was added and stirring at rt was continued for 35 min. DIEA (0.262 mL, 1.500 mmol) was added and stirring at rt was continued for 55 min. H₂O (0.5 mL) was added and the reaction mixture was concentrated in vacuo. The obtained residue was partitioned between EtOAc (60 mL) and 1 M aq. HCl (15 mL). The organic layer was washed with 5% aq. KHSO₄ (3×15 mL) and brine (15 mL), dried over Na₂SO₄, filtered, and concentrated to dryness in vacuo.

The crude product was purified by silica gel flash chromatography (Part 1, Eluent A: DCM; Part 2, Eluent A: Heptane/AcOH (99:1), Eluent B: EtOAc/AcOH (99:1)). Pure fractions were combined and concentrated to dryness in vacuo. The residue was dissolved in EtOAc (50 mL) and the solution was washed with 5% aq. NaHCO₃ (3×5 mL), 5% aq. KHSO₄ (1×10 mL) and brine (10 mL), dried over Na₂SO₄, filtered, and concentrated to dryness in vacuo. The residue was lyophilized from tert-BuOH/H₂O (4:1) to afford G11 (434 mg, 0.741 mmol, 49.4% yield for 2 steps) as a white solid. Analytical method 10; t_{R}=1.36 min; [M+H]⁺=586.5.

### Example 13: Building Block H—Benzylated CDE-Trimer

### Example 13.1: Synthesis of (S)-3-((S)-2-((S)-2-((4-chloro-2-(4-(2-((dimethylamino)methyl)-1-methyl-1H-imidazol-5-yl)phenoxy)benzyl)amino)propanamido)-3-methoxy-N-methylpropanamido)-4-(4-chlorophenyl)butanoic acid (H1)

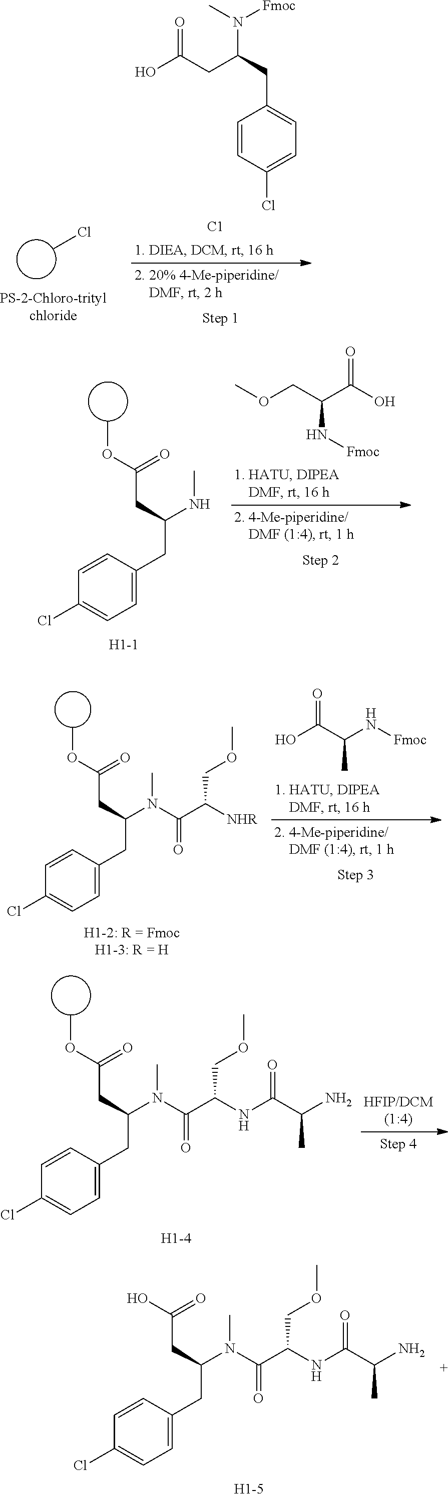

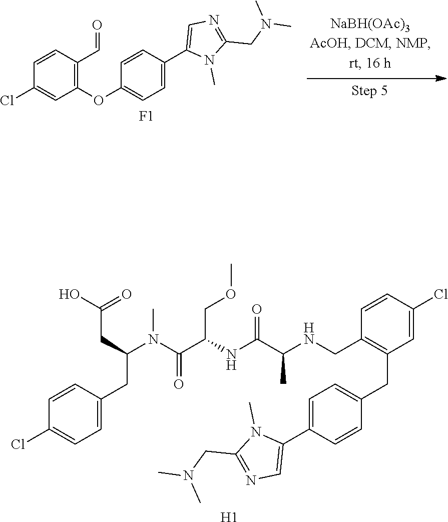

### Step 1. PS-2-chloro-trityl (S)-4-(4-chlorophenyl)-3-(methylamino)butanoate (H1-1)

Step 1-1. 2-Chlorotrityl chloride PS resin (49.8 g, 49.8 mmol) was pre-washed with DCM (3×200 mL). A solution of C1 (20.0 g, 44.5 mmol) and DIPEA (17.0 mL, 97 mmol) dissolved in DCM (100 mL) was added to the resin. The resulting mixture was shaken at rt for 16 h, then washed with DCM (3×200 mL), and shaken in DCM/MeOH (5:2) (70 mL) to cap resin for 30 min. The resin was then filtered, washed with DMF (2×200 mL) and DCM (2×200 mL), and dried under vacuum.

Step 1-2. To dried resin (70.0 g) was added 20% 4-methylpiperidine in DMF (300 mL) and the mixture was shaken at rt for 2 h. The resin was filtered, washed with DMF (4×100 mL) and DCM (4×100 mL), and dried under vacuum. Resin H1-1 (60 g, crude) was taken onto the next step without purification.

### Step 2. PS-2-chloro-trityl (S)-3-((S)-2-amino-3-methoxy-N-methylpropanamido)-4-(4-chlorophenyl)butanoate (H1-3)

Step 2-1. To H1-1 (60.0 g, 48.0 mmol) in DMF (300 mL) was added a pre-mixed solution of Fmoc-L-Ser(OMe)-OH (19.66 g, 57.6 mmol), DIPEA (20.96 mL, 120.0 mmol) and HATU (21 g, 55.2 mmol). The reaction mixture was shaken for 18 h at rt. The resin was filtered, washed with DMF (2×200 mL) and DCM (2×200 mL) and dried under vacuum to yield H1-2.

Step 2-2. H1-3 (72 g, crude) was prepared according to the procedure used for Example 13.1, Step 1-2 which was taken onto the next step without purification.

### Step 3. PS-2-chloro-trityl (S)-3-((S)-2-((S)-2-aminopropanamido)-3-methoxy-N-methylpropanamido)-4-(4-chlorophenyl)butanoate (H1-4)

H1-4 resin (66 g, crude) was prepared according to the procedure used for Example 13.1, Step 2 starting from H1-3 (62 g, 49.6 mmol) and Fmoc-Ala-OH (18.53 g, 59.5 mmol). H1-4 resin was taken onto the next step without purification.

### Step 4. (S)-3-((S)-2-((S)-2-aminopropanamido)-3-methoxy-N-methylpropanamido)-4-(4-chlorophenyl) butanoic acid (H1-5)

H1-4 (66 g, 52.8 mmol) was cleaved from the resin by shaking with 400 mL of 20% HFIP/DCM for 30 min at rt. The resin was drained and the filtrate collected. This was repeated 3 more times. The combined filtrates were concentrated to provide H1-5 (22.29 g, 98%) as a pink foam. Analytical method 2; t_{R}=0.65 min; [M+H]⁺=400.2.

### Step 5. (S)-3-((S)-2-((S)-2-((4-chloro-2-(4-(2-((dimethylamino)methyl)-1-methyl-1H-imidazol-5-yl) phenoxy)benzyl)amino)propanamido)-3-methoxy-N-methylpropanamido)-4-(4-chlorophenyl)butanoic acid (H1)

To Intermediate H1-5 (2.2 g, 5.5 mmol) dissolved in DCM (50 mL) and NMP (10 mL) was added Intermediate F1 (1.628 g, 4.40 mmol), followed by acetic acid (0.472 mL, 8.25 mmol) and the resulting mixture was stirred at rt for 1.5 h. Sodium triacetoxyborohydride (0.624 g, 16.51 mmol) was then added and stirring was continued for 16 h at rt. The reaction mixture was quenched with MeOH/water (1 mL) and stirred until gas evolution stopped. The mixture was concentrated and purified directly via reversed-phase chromatography (eluting with MeCN/water with 0.1% NH₄OH) to afford H1 (2.7 g, 63.7%). Analytical method 5; t_{R}=0.73 min; [M+H]⁺=753.2.

The Building Blocks (BB) H2 to H4 shown in Table 33 below were prepared according to the procedure used for H1 (Example 13.1).

TABLE 33
Benzylated CDE trimers

| BB | | | |
|---|---|---|---|
| No. | Structure | Name | LCMS |
| H2 | 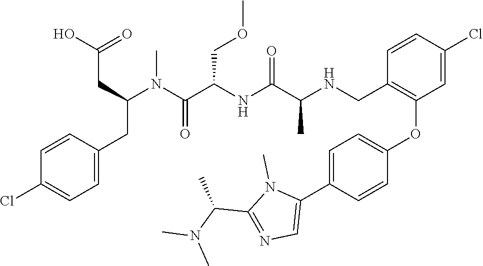 [SMILES: COC[C@H](NC(=O)[C@H](C)NCc1ccc(Cl)cc1Oc1ccc(-c2cnc([C@@H](C)N(C)C)n2C)cc1)C(=O)N(C)[C@H](CC(=O)O)Cc1ccc(Cl)cc1] | (S)-3-((S)-2-((S)-2-((4- chloro-2-(4-(2-((R)-1- (dimethylamino)ethyl)-1- methyl-1H-imidazol-5- yl)phenoxy)benzyl)amino) propanamido)-3-methoxy- N-methylpropanamido)-4- (4-chlorophenyl)butanoic acid | Analytical method 5 t_{R} = 0.73 min [M + H]⁺ = 767.0 |
| H3 | 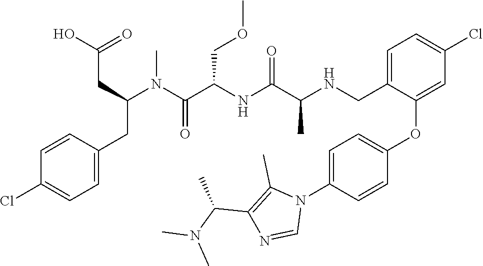 [SMILES: COC[C@H](NC(=O)[C@H](C)NCc1ccc(Cl)cc1Oc1ccc(-n2cnc([C@@H](C)N(C)C)c2C)cc1)C(=O)N(C)[C@H](CC(=O)O)Cc1ccc(Cl)cc1] | (S)-3-((S)-2-((S)-2-((4- chloro-2-(4-(4-((R)-1- (dimethylamino)ethyl)-5- methyl-1H-imidazol-1- yl)phenoxy)benzyl)amino) propanamido)-3-methoxy-N- methylpropanamido)-4-(4- chlorophenyl)butanoic acid | Analytical method 5 t_{R} = 0.77 min [M + H]⁺ = 767.0 |
| H4 | 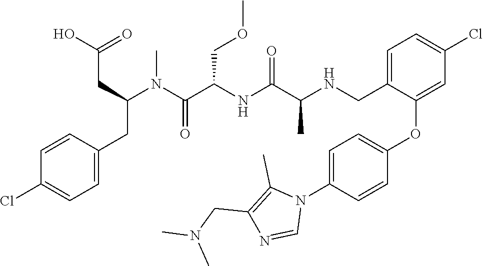 [SMILES: COC[C@H](NC(=O)[C@H](C)NCc1ccc(Cl)cc1Oc1ccc(-n2cnc(CN(C)C)c2C)cc1)C(=O)N(C)[C@H](CC(=O)O)Cc1ccc(Cl)cc1] | (S)-3-((S)-2-((S)-2-((4- chloro-2-(4-(4- ((dimethylamino)methyl)-5- methyl-1H-imidazol-1- yl)phenoxy)benzyl)amino) propanamido)-3-methoxy-N- methylpropanamido)-4-(4- chlorophenyl)butanoic acid | Analytical method 5 t_{R} = 0.76 min [M + H]⁺ = 753.2 |

### Example 14: Building block J—Fmoc-protected CDE trimer

### Example 14.1 Synthesis of (5S,8S,11S)-11-(4-chlorobenzyl)-1-(9H-fluoren-9-yl)-8-(methoxymethyl)-5,10-dimethyl-3,6,9-trioxo-2-oxa-4,7,10-triazatridecan-13-oic acid (J1)

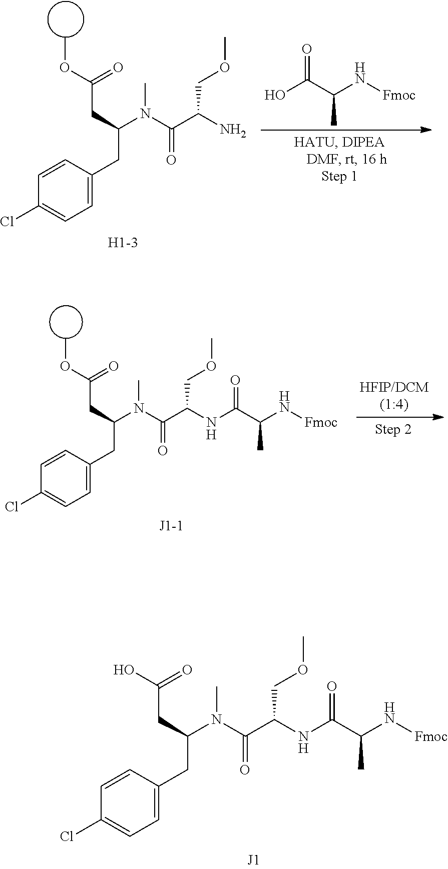

J1 (2 g, 64%) was prepared according to the procedure used for Example 13.1, Step 3-1 and Step 4 starting from intermediate H1-3 (10 g, 5 mmol). Analytical method 1; t_{R}=1.50 min; [M−H]⁻=622.6.

J2 in Table 34 below was prepared according to the procedure used for J1 (Example 14.1).

TABLE 34
Fmoc-protected CDE trimer J2

| BB | | | |
|---|---|---|---|
| No. | Structure | Name | LCMS |
| J2 | 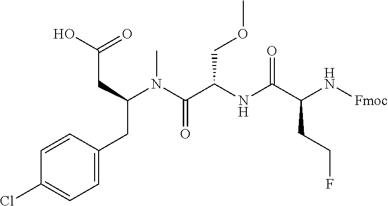 | (5S,8S,11S)-11-(4- chlorobenzyl)-1-(9H- fluoren-9-yl)-5-(2- fluoroethyl)-8- (methoxymethyl)-10- methyl-3,6,9-trioxo-2-oxa- 4,7,10-triazatridecan- 13-oic acid | Analytical method 5 t_{R} = 0.82 min [M + H]⁺ = 654.1 |

### Example 15: Building Block K—ABC Trimer

### Example 15.1: (5S,10R,13R)-13-benzyl-5-(4-chlorobenzyl)-1-(9H-fluoren-9-yl)-10-(methoxycarbonyl)-4,11-dimethyl-3,7,12-trioxo-2-oxa-4,8,11-triazapentadecan-15-oic acid (K1)

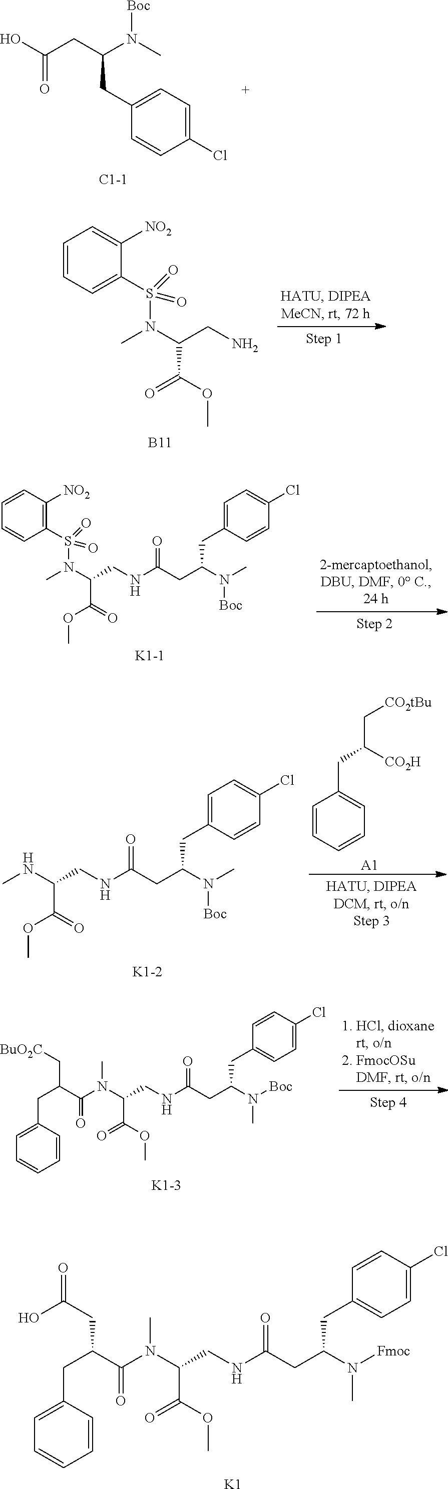

### Step 1. (R)-methyl 3-((S)-3-((tert-butoxycarbonyl)(methyl)amino)-4-(4-chlorophenyl)butanamido)-2-(N-methyl-2-nitrophenylsulfonamido)propanoate (K1-1)

To a solution of C1-1 (2.39 g, 7.29 mmol) in MeCN (50 mL) was added B11 (6.92 g, 6.65 mmol) dissolved in MeCN (40 mL). HATU (2.78 g, 7.32 mmol) and DIPEA (5.8 mL, 33.2 mmol) and the resulting mixture was stirred at rt for ˜2 days. The reaction mixture was concentrated. The resulting residue was diluted with EtOAc and transferred to a separatory funnel. The organic phase was washed with 1 M HCl, sat. NaHCO₃ solution, and brine, dried over MgSO₄, filtered, and concentrated in vacuo. The crude product was purified via silica flash column chromatography (eluting with 25→75% EtOAc in heptane) to obtain K1-1 as a yellow foamy solid (5.80 g, 85%). Analytical method 1; t=1.19 min, [M+H]0=671.1.

### Step 2. (R)-methyl 3-((S)-3-((tert-butoxycarbonyl)(methyl)amino)-4-(4-chlorophenyl) butanamido)-2-(methylamino)propanoate (K1-2)

To a solution of K1-1 (5.41 g, 5.26 mmol) in DMF (56 mL) and cooled to 0° C. was added dropwise DBU (4 mL, 26.5 mmol) dropwise, followed by 2-mercaptoethanol (3.7 mL, 52.6 mmol) and the resulting mixture was stirred at 0° C. for 24 h. The reaction mixture was then quenched with sat. NaHCO₃ solution, partitioned with EtOAc, and then transferred to a separatory funnel. The phases were separated, and the aqueous phase was extracted with EtOAc (5×). The combined organic phases were dried over MgSO₄, filtered, and concentrated in vacuo. Purification of the crude product via silica flash column chromatography (eluting with 0→10% MeOH in DCM) provided K1-2 as a yellow oil (3.98 g, 91%). Analytical method 1; t_{R}=0.80 min; [M+H]⁺=442.2.

### Step 3. (6S,11R,14R)-tert-butyl 14-benzyl-6-(4-chlorobenzyl)-11-(methoxycarbonyl)-2,2,5,12-tetramethyl-4,8,13-trioxo-3-oxa-5,9,12-triazahexadecan-16-oate (K1-3)

To a solution of Amine K1-2 (3.98 g, 4.77 mmol) in DCM (53 mL) was added succinic acid A1 (1.91 g, 7.23 mmol), HATU (2.72 g, 7.16 mmol), and DIPEA (3.5 mL, 20.04 mmol) and the resulting mixture was stirred at room temperature overnight. The reaction mixture was then concentrated to remove the DCM. The resulting residue was taken up in EtOAc and transferred to a separatory funnel. The phases were separated. The organic phase was washed with 1 M HCl and sat. NaHCO₃, dried over MgSO₄, filtered, and concentrated in vacuo. Silica flash column chromatography (eluting with 10% MeOH/DCM) provided K1-3 as a tan foamy solid (3.53 g, 71%). Analytical method 5; t_{R}=1.32 min, [M+H]⁺=688.5.

### Step 4. (5S,10R,13R)-13-benzyl-5-(4-chlorobenzyl)-1-(9H-fluoren-9-yl)-10-(methoxycarbonyl)-4,11-dimethyl-3,7,12-trioxo-2-oxa-4,8,11-triazapentadecan-15-oic acid (K1)

To K1-3 (3.53 g, 3.39 mmol) was added HCl in dioxane (12 mL, 48.0 mmol), and the resulting mixture was stirred at rt overnight. Volatiles were then removed in vacuo, and a tan foamy solid was obtained (3.45 g). The resulting crude product (348 mg, 0.654 mmol) and Fmoc-OSu (331 mg, 0.981 mmol) were taken up in DMF (7 mL) and the resulting mixture was stirred at rt overnight. The volatiles were then removed in vacuo. Silica flash column chromatography of the residue (eluting with 0→10% MeOH in DCM) provided K1 as a colorless gum (215 mg, 43%). Analytical method 1; t_{R}=1.08 min, [M+H]⁺=754.4.

### Example 16: PCSK9 Ligand Binding Assay

The PCSK9 binding of the compounds of the disclosure were measured using a time resolved fluorescence resonance energy transfer (TR-FRET) assay. This time resolved fluorescence resonance energy transfer (TR-FRET) assay measures the ability of a compound of the present disclosure to interfere with the binding of human PCSK9 to human LDLR, providing measures of both potency (IC50) and efficacy (Amax).

### Materials

Human PCSK9
Human PCSK9 Alexa Fluor 647
Human LDLR extracellular domain-Europium Kryptate
Proxi plate-low volume assay plate (PerkinElmer #6008280)
Greiner V-bottom (Greiner BioOne #781280)
Assay Buffer
   20 mM HEPES, pH 7.5
   150 mM NaCl
   1 mM CaCl₂
   0.01% v/v Tween20
   0.01% w/v BSA

A master compound plate was prepared in a Greiner V bottom plate by diluting compounds of the disclosure in dimethylsulfoxide to the correct concentration for the desired top concentration based on the desired final concentration: for a 30 uM final concentration the master plate concentration is 1.5 mM (68 uL DMSO+12 uL 10 mM of a compound of the disclosure), for a 10 uM final concentration the master plate concentration is 0.5 mM (76 uL DMSO+4 uL 10 mM of a compound of the disclosure), for a 3 uM final concentration the master plate concentration is 150 uM (69 uL DMSO+1 uL 10 mM of a compound of the disclosure). These solutions were pipetted into columns 1 and 11 of the compound plate. Threefold serial dilutions were generated in columns 2-10 and 12-20 of the compound plate by transferring 10 uL into 20 uL of DMSO. Columns 21 and 22 of the compound plate were negative controls containing DMSO alone.

An intermediate plate was generated in a Greiner V bottom plate by transferring 8 uL from each well of the master plate into a corresponding well containing 92 uL of assay buffer and mixing thoroughly.

A Proxi plate-low volume assay plate was used for the assay. To all wells of the plate was added 10 uL of 16 nM Human PCSK9 Alexa Fluor 647, followed by 5 uL from the intermediate plate. For the positive control wells in columns 23 and 24 of the plate, 5 uL of unlabeled human PCSK9 was added at 4 uM in assay buffer containing 8% DMSO. Following a 30 minute incubation, 5 uL of 4 nM Human LDLR extracellular domain-Europium Kryptate was added and the mixture was incubated for an additional 2 hours.

The TR-FRET signal was measured on an EnVision or PheraStar instrument with a 60 μs delay, 330 nm excitation and 665 nm emission (FRET), and 330 nM excitation and 615 nm (Europium). The FRET ratio (FRET/Europium) was used for calculations.

### Data Analysis

No inhibition (0%) was observed from the wells containing DMSO (Control) in columns 21 and 22 of the compound plate. Full inhibition (100%) was observed from the wells containing 1 uM human PCSK9 (Control) in columns 23 and 24 of the plate. Data is expressed as percent inhibition: (value−0%)/(100%−0%). Table 35 shows the PCSK9 activity of cyclic polypeptides of the present disclosure.

TABLE 35
PCSK9 activity of cyclic polypeptides of the
present disclosure in the PCSK9 Fret assay.

| Cmd | PCSK9 FRET Assay | Max % |
|---|---|---|
| No. | IC₅₀ (uM) | inhibition |
| 2 | 0.1722 | 95.16 |
| 3 | 0.0013 | 95.68 |
| 4 | 0.0029 | 97.50 |
| 5 | 0.0096 | 83.48 |
| 7 | 0.0099 | 93.00 |
| 8 | 0.0089 | 101.12 |
| 9 | 0.0031 | 98.16 |
| 10 | 0.0043 | 101.88 |
| 11 | 0.0026 | 105.07 |
| 12 | 0.0741 | 98.09 |
| 13 | 0.1062 | 101.32 |
| 14 | 0.3073 | 93.84 |
| 15 | 0.3308 | 48.77 |
| 16 | 0.0017 | 92.71 |
| 17 | 0.0612 | 81.08 |
| 18 | 0.0011 | 86.12 |
| 19 | 0.0015 | 106.65 |
| 20 | 0.0012 | 98.33 |
| 21 | 0.0024 | 66.09 |
| 22 | 0.0011 | 98.82 |
| 23 | 0.0022 | 96.76 |
| 25 | 0.1132 | 52.86 |
| 26 | 0.0024 | 104.11 |
| 27 | 0.0011 | 101.53 |
| 28 | 0.0036 | 102.04 |
| 29 | 0.0006 | 94.90 |
| 30 | 0.0598 | 85.21 |
| 31 | 0.7868 | 60.09 |
| 32 | 0.0018 | 103.15 |
| 33 | 0.0013 | 95.65 |
| 34 | 0.0010 | 95.86 |
| 35 | 0.0042 | 104.05 |
| 36 | 0.0027 | 82.38 |
| 37 | 0.0023 | 100.65 |
| 38 | 0.0021 | 101.48 |
| 39 | 0.0009 | 104.01 |
| 40 | 0.0996 | 104.99 |
| 41 | 0.0047 | 84.34 |
| 42 | 0.0245 | 102.18 |
| 43 | 0.0018 | 94.38 |
| 44 | 0.0026 | 102.15 |
| 45 | 0.0022 | 95.96 |
| 46 | 0.0027 | 96.56 |
| 48 | 0.0048 | 87.82 |
| 49 | 0.0017 | 99.12 |
| 50 | 0.0410 | 101.21 |
| 51 | 0.0005 | 94.86 |
| 52 | 0.0015 | 104.32 |
| 53 | 0.0211 | 92.32 |
| 54 | 0.0046 | 92.54 |
| 55 | 0.0093 | 58.77 |
| 56 | 0.0026 | 101.80 |
| 57 | 0.0021 | 95.83 |
| 58 | 0.0093 | 103.43 |
| 60 | 0.0029 | 82.23 |
| 61 | 0.0014 | 99.31 |
| 62 | 0.0168 | 88.46 |
| 63 | 0.0873 | 103.93 |
| 64 | 0.0090 | 79.08 |
| 65 | 0.1886 | 92.63 |
| 66 | 0.0772 | 91.88 |
| 67 | 0.0006 | 96.63 |
| 68 | 0.0305 | 77.84 |
| 69 | 0.0430 | 64.09 |
| 70 | 0.0009 | 101.10 |
| 71 | 0.0129 | 102.12 |
| 72 | 0.0005 | 92.57 |
| 73 | 0.0399 | 90.77 |
| 74 | 0.0465 | 104.33 |
| 75 | 0.0040 | 103.69 |
| 76 | 0.0010 | 96.97 |
| 77 | 0.0090 | 104.08 |
| 78 | 0.0946 | 97.49 |
| 79 | 0.0018 | 103.04 |
| 80 | 0.0063 | 103.62 |
| 81 | 0.0345 | 87.86 |
| 82 | 0.0013 | 99.99 |
| 83 | 0.0017 | 99.18 |
| 84 | 0.0032 | 95.65 |
| 85 | 0.0009 | 114.68 |
| 86 | 0.0205 | 56.62 |
| 87 | 0.4968 | 87.74 |
| 88 | 0.0401 | 90.30 |
| 89 | 0.0016 | 85.89 |
| 90 | 0.0021 | 101.52 |
| 91 | 0.1682 | 86.95 |
| 92 | 0.0646 | 61.54 |
| 93 | 0.0063 | 96.70 |
| 94 | 0.0024 | 104.90 |
| 95 | 0.0008 | 104.58 |
| 96 | 0.3043 | 78.14 |
| 97 | 0.0022 | 90.83 |
| 98 | 0.0019 | 98.16 |
| 99 | 0.0016 | 104.20 |
| 100 | 0.0054 | 105.41 |
| 101 | 0.0011 | 94.32 |
| 102 | 0.0015 | 101.56 |
| 103 | 0.0101 | 106.00 |
| 104 | 0.0014 | 109.94 |
| 105 | 0.0010 | 104.44 |
| 106 | 0.0020 | 106.45 |
| 107 | 0.0017 | 100.41 |
| 108 | 0.0007 | 99.73 |
| 109 | 0.0076 | 76.18 |
| 110 | 0.0012 | 99.22 |
| 111 | 0.0009 | 103.17 |
| 112 | 0.0032 | 107.46 |
| 113 | 0.2992 | 53.35 |
| 114 | 0.0045 | 57.46 |
| 115 | 0.0053 | 72.72 |
| 116 | 0.0048 | 59.45 |
| 117 | 0.0042 | 67.25 |
| 118 | 0.0246 | 65.13 |
| 119 | 0.0035 | 64.72 |
| 120 | 0.0236 | 48.04 |
| 121 | 0.0028 | 59.89 |
| 122 | 0.0087 | 55.05 |
| 123 | 0.0034 | 74.19 |
| 124 | 0.0038 | 100.50 |
| 125 | 0.0280 | 60.93 |
| 126 | 0.0015 | 101.12 |
| 127 | 0.0078 | 55.01 |
| 128 | 0.0204 | 51.35 |
| 129 | 0.0063 | 94.48 |
| 130 | 0.0203 | 77.42 |
| 131 | 0.0099 | 90.72 |
| 132 | 0.0073 | 71.97 |
| 133 | 0.0058 | 93.15 |
| 134 | 0.0395 | 98.77 |
| 135 | 0.0094 | 58.33 |
| 136 | 0.0050 | 96.16 |
| 137 | 0.0084 | 90.76 |
| 138 | 0.0015 | 98.02 |
| 139 | 0.0057 | 65.69 |
| 141 | 0.8817 | 102.37 |
| 142 | 0.0012 | 100.91 |
| 143 | 0.7742 | 49.36 |
| 144 | 0.0070 | 100.34 |
| 145 | 0.0011 | 102.40 |
| 146 | 0.0031 | 51.67 |
| 147 | 0.0037 | 104.16 |
| 148 | 0.0292 | 94.67 |
| 149 | 0.0187 | 72.43 |
| 150 | 0.0008 | 104.13 |
| 151 | 0.0088 | 64.23 |
| 152 | 0.0012 | 107.64 |
| 154 | 0.0051 | 80.05 |
| 155 | 0.0008 | 98.43 |
| 156 | 0.0037 | 108.38 |
| 157 | 0.0018 | 89.28 |
| 158 | 0.0014 | 98.98 |
| 159 | 0.0740 | 55.26 |
| 160 | 0.0076 | 91.45 |
| 161 | 0.0147 | 92.62 |
| 162 | 0.0029 | 91.41 |
| 163 | 0.1347 | 84.60 |
| 164 | 0.0036 | 80.18 |
| 165 | 0.0056 | 92.29 |
| 166 | 0.0043 | 96.70 |
| 167 | 0.0018 | 81.26 |
| 168 | 0.0677 | 85.66 |
| 169 | 0.0013 | 91.73 |
| 170 | 0.0013 | 102.49 |
| 171 | 0.0031 | 91.76 |
| 172 | 0.0014 | 102.44 |
| 173 | 0.0733 | 84.80 |
| 174 | 0.0114 | 102.02 |
| 175 | 0.0058 | 102.55 |
| 176 | 0.0043 | 70.99 |
| 177 | 0.0098 | 107.53 |
| 178 | 0.0023 | 114.16 |
| 179 | 0.0006 | 103.76 |
| 180 | 0.0031 | 102.27 |
| 181 | 0.0103 | 54.93 |
| 182 | 0.0012 | 99.86 |
| 183 | 0.0112 | 81.31 |
| 184 | 0.0094 | 54.06 |
| 185 | 0.0115 | 96.86 |
| 186 | 0.0006 | 103.39 |
| 187 | 0.0013 | 94.30 |
| 188 | 0.0162 | 75.17 |
| 189 | 0.0014 | 95.01 |
| 190 | 0.0326 | 100.60 |
| 191 | 0.0021 | 94.20 |
| 192 | 0.1066 | 97.97 |
| 193 | 0.0061 | 84.54 |
| 194 | 0.0055 | 96.92 |
| 195 | 0.0734 | 100.43 |
| 196 | 0.0030 | 97.92 |
| 197 | 0.0174 | 72.51 |
| 198 | 0.0006 | 98.27 |
| 199 | 0.0248 | 100.29 |
| 200 | 0.0654 | 93.78 |
| 201 | 0.4414 | 72.22 |
| 202 | 0.0029 | 95.27 |
| 203 | 0.0020 | 90.79 |
| 204 | 0.2859 | 83.07 |
| 205 | 0.0020 | 93.20 |
| 206 | 0.0058 | 99.72 |
| 207 | 0.0022 | 102.49 |
| 208 | 0.0027 | 87.45 |
| 209 | 0.0049 | 98.10 |
| 210 | 0.0147 | 91.38 |
| 211 | 0.0039 | 102.01 |
| 212 | 0.0054 | 71.82 |
| 213 | 0.0035 | 91.51 |
| 214 | 0.0332 | 97.43 |
| 215 | 0.0110 | 95.08 |
| 216 | 0.0006 | 109.48 |
| 217 | 0.0007 | 95.74 |
| 218 | 0.0010 | 105.97 |
| 219 | 0.0009 | 105.15 |
| 220 | 0.0021 | 94.82 |
| 221 | 0.0014 | 98.35 |
| 222 | 0.0026 | 50.13 |
| 223 | 0.0018 | 100.16 |
| 224 | 0.6495 | 95.15 |
| 225 | 0.0080 | 98.86 |
| 226 | 0.2493 | 42.24 |
| 227 | 0.0088 | 104.70 |
| 228 | 0.0084 | 93.92 |
| 229 | 0.0006 | 99.91 |
| 230 | 0.0778 | 46.03 |
| 232 | 0.0057 | 96.17 |
| 233 | 0.7049 | 90.08 |
| 234 | 0.0008 | 100.63 |
| 235 | 0.0111 | 100.56 |
| 236 | 0.0066 | 95.33 |
| 237 | 0.0014 | 102.97 |
| 238 | 0.0025 | 104.90 |
| 239 | 0.0013 | 106.17 |
| 240 | 0.0009 | 99.28 |
| 241 | 0.2469 | 72.66 |
| 242 | 0.0013 | 87.34 |
| 243 | 0.0009 | 97.38 |
| 244 | 0.0229 | 80.79 |
| 245 | 0.0013 | 104.03 |
| 246 | 0.0024 | 103.97 |
| 247 | 0.0332 | 98.86 |
| 248 | 0.0022 | 106.74 |
| 249 | 0.0023 | 98.36 |
| 250 | 0.0004 | 84.24 |
| 251 | 0.0008 | 99.24 |
| 252 | 0.0021 | 92.00 |
| 253 | 0.0015 | 98.40 |
| 254 | 0.0030 | 102.70 |
| 255 | 0.0197 | 64.95 |
| 256 | 0.0524 | 79.36 |
| 257 | 0.0213 | 50.23 |
| 258 | 0.0043 | 98.94 |
| 259 | 0.0032 | 108.15 |
| 260 | 0.0021 | 109.05 |
| 261 | 0.0020 | 111.41 |
| 262 | 0.2052 | 97.63 |
| 263 | 0.0053 | 76.79 |
| 265 | 0.3124 | 104.88 |
| 266 | 0.0019 | 106.30 |
| 267 | 0.0071 | 93.76 |
| 268 | 0.0094 | 68.37 |
| 269 | 0.0013 | 97.18 |
| 270 | 0.0027 | 106.63 |
| 271 | 0.0069 | 84.13 |
| 272 | 0.0012 | 98.30 |
| 273 | 0.0030 | 105.53 |
| 274 | 0.0014 | 93.20 |
| 275 | 0.0008 | 102.47 |
| 276 | 0.0164 | 101.53 |
| 277 | 0.0025 | 83.22 |
| 278 | 0.0013 | 107.17 |
| 279 | 0.0007 | 105.26 |
| 280 | 0.0006 | 100.09 |
| 281 | 0.0010 | 89.62 |
| 282 | 0.0061 | 60.78 |
| 283 | 0.0016 | 105.87 |
| 284 | 0.0009 | 98.50 |
| 285 | 0.0021 | 90.24 |
| 286 | 0.0007 | 59.34 |
| 287 | 0.0026 | 89.28 |
| 288 | 0.0241 | 87.05 |
| 289 | 0.0006 | 100.92 |
| 290 | 0.0036 | 79.21 |
| 291 | 0.0016 | 102.86 |
| 292 | 0.0030 | 113.30 |
| 293 | 0.0007 | 103.68 |
| 294 | 0.1046 | 55.44 |
| 295 | 0.0018 | 101.64 |
| 296 | 0.0012 | 98.03 |
| 298 | 0.00201 | 99.00 |
| 299 | 0.0033 | 95.40 |
| 300 | 0.0021 | 80.60 |
| 301 | 0.0039 | 54.20 |
| 302 | 0.0035 | 54.00 |
| 303 | 0.0029 | 52.60 |
| 304 | 0.0006 | 88.80 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A compound of Formula (I): 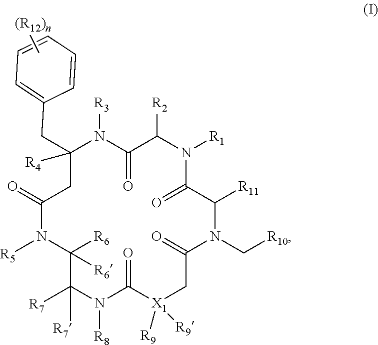
wherein:
X₁ is C or N;
R₁ is H, (C₁-C₆)alkyl, or (C₁-C₆)haloalkyl; or
R₁ and R₁₁ together with the atoms to which they are attached form a 5- to 7-membered heterocyclyl ring comprising 1-3 heteroatoms selected from N, O, and S optionally substituted with one or more substituents each independently selected from ═(O), (C₁-C₆)alkyl, and (C₁-C₆)haloalkyl;
R₂ is (C₁-C₆)alkyl, (C₁-C₆)haloalkyl, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one or more substituents each independently selected from (C₁-C₆)alkoxy, (C₁-C₆)haloalkoxy, —C(O)(C₁-C₆)alkyl, —C(O)OH, —C(O)O(C₁-C₆)alkyl, —OC(O)(C₁-C₆)alkyl, —C(O)NR₁₇R₁₈, —NR₁₇C(O)R₁₈, (C₆-C₁₀)aryl, and 5- or 6-membered heteroaryl comprising 1-4 heteroatoms selected from N, O, and S;
R₃, R₄, and R₅ are each independently H, (C₁-C₆)alkyl, or (C₁-C₆)haloalkyl;
R₆, R_{6′}, R₇, and R_{7′} are each independently H, (C₁-C₆)alkyl, (C₁-C₆)haloalkyl, (C₁-C₆)hydroxyalkyl, —C(O)OH, or —C(O)O(C₁-C₆)alkyl, (C₁-C₆)hydroxyalkyl, wherein the alkyl is optionally substituted with one or more substituents each independently selected from (C₁-C₆)alkoxy, —C(O)OH, —C(O)O(C₁-C₆)alkyl, —NR₁₇R₁₈, —C(O)NR₁₇R₁₈, —NR₁₇C(O)R₁₈, (C₃-C₇)cycloalkyl, and 4- to 7-membered heterocyclyl comprising 1-3 heteroatoms selected from N, O, and S; or
R₆ and R₇ together with the carbon atoms to which they are attached form a (C₃-C₇)cycloalkyl or a 4- to 7-membered heterocyclyl ring comprising 1-3 heteroatoms selected from N, O, and S; or
R₇ and R_{7′} together with the carbon atom to which they are attached form a (C₃-C₇)cycloalkyl or a 4- to 7-membered heterocyclyl ring comprising 1-3 heteroatoms selected from N, O, and S; or
R₇ and R₉ together with the atoms to which they are attached form a 5- to 7-membered heterocyclyl ring comprising 1-3 heteroatoms selected from N, O, and S optionally substituted with one or more substituents each independently selected from (C₁-C₆)alkyl, (C₁-C₆)haloalkyl, and ═(O);
R₈ is H or (C₁-C₆)alkyl;
R₉ and R_{9′} are each independently H, (C₁-C₆)alkyl, (C₂-C₆)alkenyl, (C₁-C₆)haloalkyl, (C₂-C₆)haloalkenyl, (C₁-C₆)alkoxy, (C₁-C₆)haloalkoxy, (C₁-C₆)hydroxyalkyl, (C₃-C₇)cycloalkyl, or 4- to 7-membered heterocyclyl comprising 1-3 heteroatoms selected from N, O, and S, wherein the alkyl is optionally substituted with one or more R₂₇; or R_{9′} is absent when X₁ is N; or
R₉ and R_{9′} together with the carbon atom to which they are attached form a (C₃-C₇)cycloalkyl or a 4- to 7-membered heterocyclyl ring comprising 1-3 heteroatoms selected from N, O, and S; or
R₇ and R₉ together with the atoms to which they are attached form a 5- to 7-membered heterocyclyl ring comprising 1-3 heteroatoms selected from N, O, and S optionally substituted with one or more substituents each independently selected from (C₁-C₆)alkyl, (C₁-C₆)haloalkyl, and ═(O);
R₁₀ is (C₆-C₁₀)aryl, 5- or 6-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl, heterocyclyl are substituted with —OR₁₃ or —NR₂₃R₁₃, and optionally substituted with one or more R₁₄;
R₁₁ is (C₁-C₆)alkyl, (C₁-C₆)haloalkyl, or (C₁-C₆)hydroxyalkyl, wherein the alkyl is optionally substituted with one or more R₁₅; or
R₁ and R₁₁ together with the atoms to which they are attached form a 5- to 7-membered heterocyclyl ring comprising 1-3 heteroatoms selected from N, O, and S optionally substituted with one or more substituents each independently selected from ═(O), (C₁-C₆)alkyl, and (C₁-C₆)haloalkyl;
R₁₂ is halogen, (C₁-C₆)alkyl, (C₁-C₆)alkoxy, (C₁-C₆)haloalkyl, (C₁-C₆)haloalkoxy, —OH, or CN;
R₁₃ is (C₆-C₁₀)aryl or 5- or 6-membered heteroaryl comprising 1-3 heteroatoms selected from N, O, and S, wherein the aryl and heteroaryl are substituted with R₁₆ and optionally substituted with one or more R₁₆;
each R₁₄ is independently at each occurrence halogen, (C₁-C₆)alkyl, (C₁-C₆)alkoxy, (C₁-C₆)haloalkyl, (C₁-C₆)haloalkoxy, oxo, —OH, or CN; or
when R₁₀ is cycloalkyl or heterocyclyl, two R₁₄ together, when attached to the same carbon atom, together form ═(O);
each R₁₅ is independently at each occurrence (C₁-C₆)alkoxy, (C₁-C₆)haloalkoxy, —C(O)R₁₉, —S(O)_{q}(C₁-C₆)alkyl, —C(O)OH, —C(O)O(C₁-C₆)alkyl, —OC(O)(C₁-C₆)alkyl, —NR₁₇R₁₈, —C(O)NR₁₇R₁₈, —NR₁₇C(O)R₂₀, —NR₁₇C(O)OR₁₈, (C₃-C₇)cycloalkyl, or 4- to 7-membered heterocyclyl comprising 1-3 heteroatoms selected from N, O, and S, wherein the cycloalkyl and heterocyclyl are optionally substituted with one or more R₂₁;
R₁₆ is —C(O)NR₃₁R₃₂, (C₆-C₁₀)aryl, or 5- to 7-membered heteroaryl comprising 1-3 heteroatoms selected from N, O, and S, wherein the aryl and heteroaryl are optionally substituted with one or more R₂₆;
each R₁₆ is independently at each occurrence halogen, (C₁-C₆)alkyl, (C₁-C₆)alkoxy, (C₁-C₆)haloalkyl, (C₁-C₆)haloalkoxy, —OH, or CN; or
R₁₆ and R_{16′} together with the atoms to which they are attached form a 5- to 7-membered heterocyclyl ring optionally substituted with one or more substituents independently selected from ═(O) and R₃₄;
R₁₇ and R₁₈ are each independently H or (C₁-C₆)alkyl optionally substituted with one or more substituents each independently selected from (C₁-C₆)alkoxy and —C(O)O(C₁-C₆)alkyl;
R₁₉ is (C₃-C₇)cycloalkyl or 4- to 7-membered heterocyclyl comprising 1-3 heteroatoms selected from N, O, and S, wherein the cycloalkyl and heterocyclyl are optionally substituted with one or more R₂₂;
R₂₀ is —(CH₂CH₂O)ₘCH₂CH₂ONH₂, —(CH₂CH₂O)ₘCH₂CH₂ONH(C₁-C₆)alkyl, or (C₁-C₆)alkyl optionally substituted with one or more —NR₂₃C(O)R₂₄;
each R₂₁ is independently at each occurrence (C₁-C₆)alkyl, (C₁-C₆)alkoxy, (C₁-C₆)haloalkyl, (C₁-C₆)haloalkoxy, halogen, ═(O), or —OH;
each R₂₂ is independently at each occurrence (C₁-C₆)alkyl, (C₁-C₆)haloalkyl, halogen, or —OH; or
two R₂₂, when on the same atom, together with the atom to which they are attached form a (C₃-C₇)spirocycloalkyl or a 4- to 7-membered spiroheterocyclyl ring comprising 1-3 heteroatoms selected from N, O, and S;
R₂₃ is H or (C₁-C₆)alkyl;
R₂₄ is H or (C₁-C₆)alkyl optionally substituted with one or more R₂₅;
each R₂₅ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with one or more substituents each independently selected from (C₁-C₆)alkyl, (C₁-C₆)haloalkyl, and ═(O);
each R₂₆ is independently at each occurrence (C₁-C₆)alkyl optionally substituted with one or more R₂₉, (C₂-C₆)alkenyl, (C₂-C₆)alkynyl, (C₁-C₆)alkoxy, (C₁-C₆)haloalkyl, (C₁-C₆)haloalkoxy, (C₁-C₆)hydroxyalkyl, —NR₃₁R₃₂, —C(O)NR₃₁R₃₂, —C(O)O(C₁-C₆)alkyl, (C₃-C₇)cycloalkyl, 4- to 7-membered heterocyclyl comprising 1-3 heteroatoms selected from N, O, and S, (C₆-C₁₀)aryl, or 5- or 6-membered heteroaryl comprising 1-3 heteroatoms selected from N, O, and S, wherein the cycloalkyl, heterocyclyl, aryl and heteroaryl are optionally substituted with one or more substituents each independently selected from (C₁-C₆)alkyl, (C₁-C₆)haloalkyl, —NH₂, —N(H)(C₁-C₆)alkyl, —N((C₁-C₆)alkyl)₂, —N(H)(C₁-C₆)haloalkyl, —N((C₁-C₆)haloalkyl)₂, halogen, and —OH; or
two R₂₆, when on adjacent atoms, together with the atoms to which they are attached form a (C₃-C₇)carbocyclyl or a 4- to 7-membered heterocyclyl ring comprising 1-3 heteroatoms selected from N, O, and S, optionally substituted with one or more R₃₃;
each R₂₇ is independently at each occurrence CN, (C₆-C₁₀)aryl, 5- to 7-membered heteroaryl comprising 1-3 heteroatoms selected from N, O, and S, (C₃-C₇)cycloalkyl, or 4- to 7-membered heterocyclyl comprising 1-3 heteroatoms selected from N, O, and S, wherein the aryl, heteroaryl, cycloalkyl and heterocyclyl are optionally substituted with one or more R₂₈;
each R₂₈ is independently at each occurrence (C₁-C₆)alkyl, (C₁-C₆)haloalkyl, (C₁-C₆)alkoxy, (C₁-C₆)haloalkoxy, (C₁-C₆)hydroxyalkyl, halogen, oxo, or CN; or
when R₂₇ is cycloalkyl or heterocyclyl, two R₂₈ together with the atoms to which they are attached form a (C₄-C₇)cycloalkyl or a 4- to 7-membered heterocyclyl ring comprising 1-3 heteroatoms selected from N, O, and S; or
when R₂₇ is cycloalkyl or heterocyclyl, two R₂₈ together, when attached to the same carbon atom, together form ═(O);
each R₂₉ is independently at each occurrence —NR₃₁R₃₂ or 4- to 7-membered heterocyclyl comprising 1-3 heteroatoms selected from N, O, and S optionally substituted with one or more R₃₀;
each R₃₀ is independently at each occurrence —OH, halogen, (C₁-C₆)alkyl, or (C₁-C₆)haloalkyl; or
two R₃₀, when on the same atom, together with the atom to which they are attached form a (C₃-C₇)spirocycloalkyl or a 4- to 7-membered spiroheterocyclyl ring comprising 1-3 heteroatoms selected from N, O, and S;
R₃₁ and R₃₂ are each independently H, (C₁-C₆)alkyl, (C₁-C₆)hydroxyalkyl, (C₃-C₇) cycloalkyl, or 4- to 7-membered heterocyclyl comprising 1-3 heteroatoms selected from N, O, and S, wherein the alkyl is optionally substituted with one or more D, and the cycloalkyl and heterocyclyl are optionally substituted with one or more substituents each independently selected from (C₁-C₆)alkyl, (C₁-C₆)haloalkyl, halogen, and —OH;
each R₃₃ is independently at each occurrence (C₁-C₆)alkyl, (C₁-C₆)haloalkyl, or —C(O)R, wherein R is (C₁-C₆)haloalkyl, (C₃-C₇)cycloalkyl, 4- to 7-membered heterocyclyl comprising 1-3 heteroatoms selected from N, O, and S, or (C₁-C₆)alkyl optionally substituted with one or more (C₁-C₆)alkoxy; or
two R₃₃, when on the same atom, together with the atom to which they are attached form a (C₃-C₇)spirocycloalkyl or a 4- to 7-membered spiroheterocyclyl ring comprising 1-3 heteroatoms selected from N, O, and S;
each R₃₄ is independently at each occurrence (C₃-C₇)cycloalkyl or 4- to 10-membered mono or bicyclic heterocyclyl comprising 1-4 heteroatoms selected from N, O, and S, wherein the cycloalkyl and heterocyclyl are optionally substituted with (C₁-C₆)alkyl optionally substituted with one or more substituents each independently selected from (C₃-C₇)cycloalkyl and 4- to 10-membered mono or bicyclic heterocyclyl comprising 1-4 heteroatoms selected from N, O, and S;
m is 1-13;
n is 1, 2, 3, or 4; and
q is 0, 1, or 2;
or a pharmaceutically acceptable salt, hydrate, solvate, stereoisomer, or tautomer thereof.

2. The compound according to claim 1, wherein X₁ is C.

3. The compound according to claim 1, wherein R₁ is H or (C₁-C₆)alkyl; or R₁ and R₁₁ together with the atoms to which they are attached form a 5- to 7-membered heterocyclyl ring comprising 1-3 heteroatoms selected from N, O, and S optionally substituted with ═(O).

4. (canceled)

5. The compound according to claim 1, wherein R₁ and R₁₁ together with the atoms to which they are attached form a 5- to 7-membered heterocyclyl ring comprising 1-3 heteroatoms selected from N, O, and S optionally substituted with ═(O).

6. The compound according to claim 1, wherein R₃, R₄, R₅, R₆, R_{6′}, and R_{7′} are each independently H or (C₁-C₆)alkyl.

7-15. (canceled)

16. The compound according to claim 1, wherein R₈ is (C₁-C₆)alkyl.

17. (canceled)

18. The compound according to claim 1, wherein R₁₂ is halogen.

19. (canceled)

20. The compound according to claim 1, wherein n is 1.

21. The compound according to claim 1, wherein n is 2.

22. The compound of claim 1, having a Formula (Ia), Formula (Ia-1), Formula (Ia-2), Formula (Ib), Formula (Ic), Formula (Id), Formula (Ie), Formula (If), Formula (Ig), Formula (Ih), Formula (Ii) or Formula (Ij):

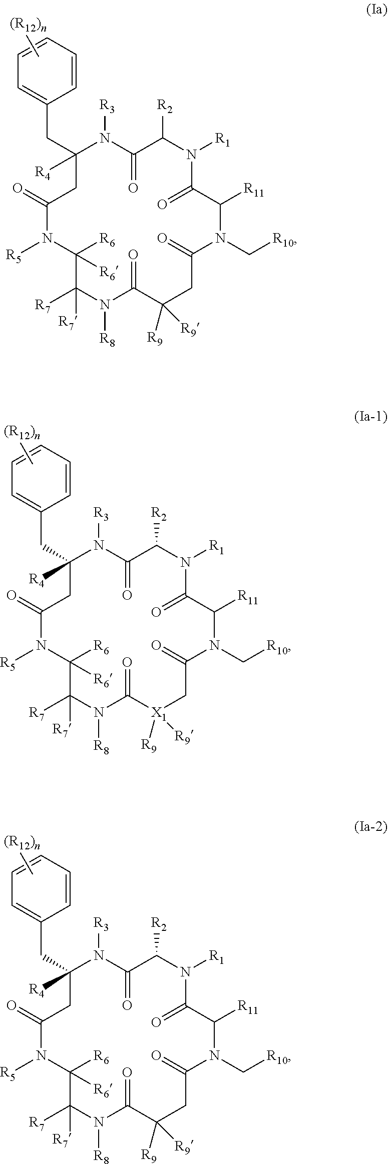

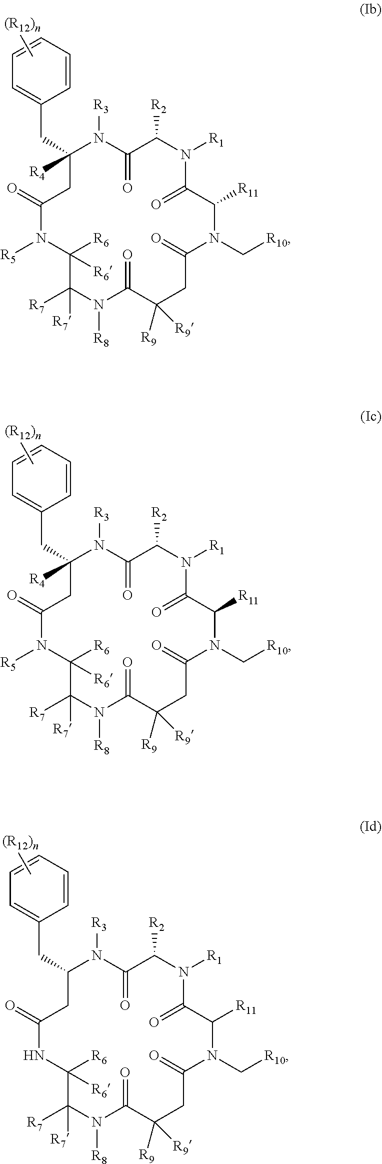

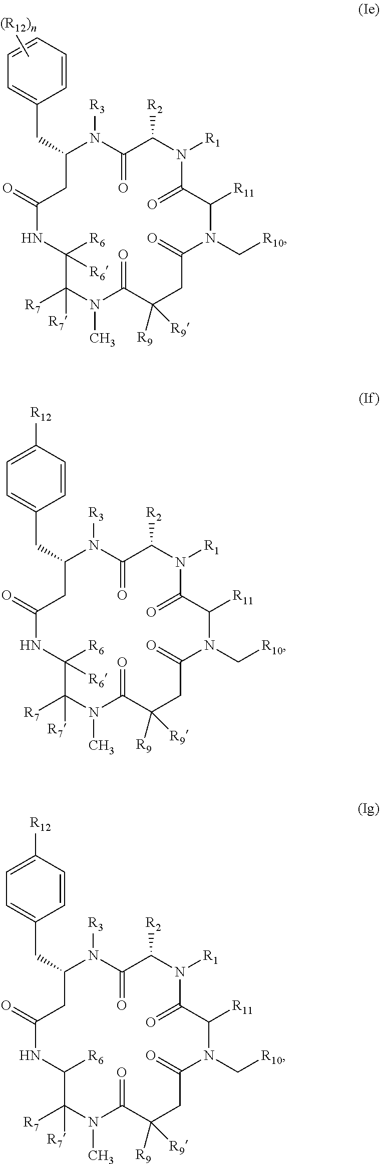

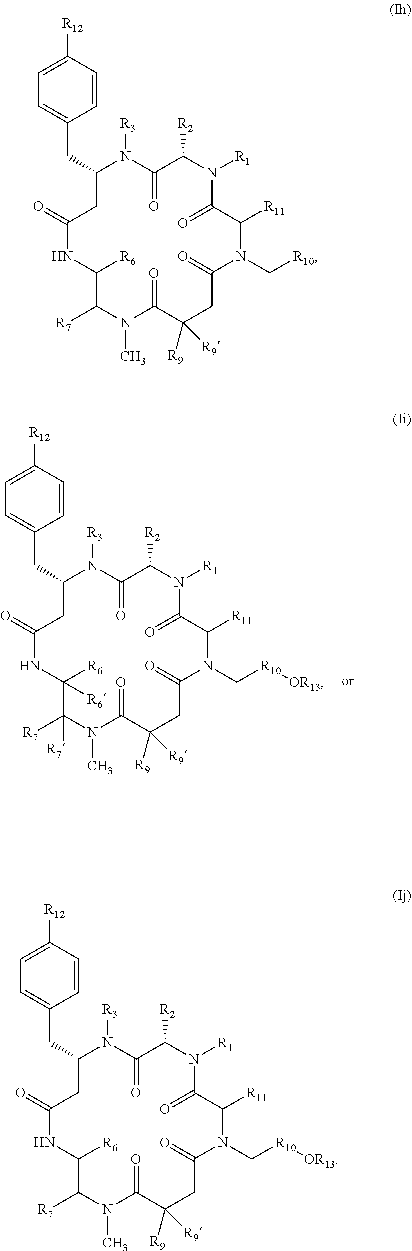

23. The compound according to claim 1, wherein R₁₀ is phenyl substituted with —OR₁₃ and optionally substituted with one to three R₁₄.

24. The compound according to claim 1, wherein R₁₀ is pyridinyl or pyridinone substituted with —OR₁₃ and optionally substituted with one to three R₁₄.

25. The compound according to claim 1, wherein R₁₃ is phenyl substituted with R₁₆ and optionally substituted with one to three R_{16′}.

26. The compound according to claim 1, wherein R₁₃ is pyridinyl substituted with R₁₆ and optionally substituted with one to three R_{16′}.

27. The compound according to claim 1 selected from:
(4S,7S,10S,14R,16aS,20aS)-14-((1H-pyrazol-1-yl)methyl)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(2-(methylamino)ethyl)hexadecahydrobenzo[l][1,4,7,11,14]pentaazacyclooctadecine-2,6,9,12,15(3H)-pentaone;
tert-butyl (3-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-3,6,10,15,18-pentaoxo-1,4,7,11,14-pentaazacyclooctadecan-2-yl)propyl)carbamate (2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-13-(cyclopropylmethyl)-5-(methoxymethyl)-2,7,14,16-tetramethyl-1,4,7,11,14-pentaazacyclooctadecane-3,6,10,15,18-pentaone;
(2S,5S,8S,13S,16R)-1-(4-chloro-2-(4-(1-methyl-2-(pyrrolidin-1-ylmethyl)-1H-imidazol-5-yl)phenoxy)-6-(2,2,2-trifluoroethoxy)benzyl)-8-(4-chlorobenzyl)-5-(methoxymethyl)-2,7,13,14,16-pentamethyl-1,4,7,11,14-pentaazacyclooctadecane-3,6,10,15,18-pentaone;
(4S,7S,10R,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7,10-bis(methoxymethyl)-5,16-dimethylhexadecahydrobenzo[l][1,4,7,11,14]pentaazacyclooctadecine-2,6,9,12,15(3H)-pentaone;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)benzyl)-5-(methoxymethyl)-2,7,13,14-tetramethyl-1,4,7,11,14-pentaazacyclooctadecane-3,6,10,15,18-pentaone;
tert-butyl 3-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-3,6,10,15,18-pentaoxo-1,4,7,11,14-pentaazacyclooctadecan-2-yl)propanoate;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2-(hydroxymethyl)-5-(methoxymethyl)-7,13,14-trimethyl-1,4,7,11,14-pentaazacyclooctadecane-3,6,10,15,18-pentaone;
(2R,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,10S,13R,17S)-13-benzyl-16-(4-chloro-2-(4-(2-((dimethylamino)methyl)-1-methyl-1H-imidazol-5-yl)phenoxy)benzyl)-5-(4-chlorobenzyl)-2-(methoxymethyl)-4,10,11-trimethyl-1,4,8,11,16-pentaazabicyclo[15.2.1]icosane-3,7,12,15,19,20-hexaone;
(4S,7S,10S,14R,16aR,20aS)-14-benzyl-11-(4-chloro-2-(4-(1-methyl-2-(pyrrolidin-1-ylmethyl)-1H-imidazol-5-yl)phenoxy)benzyl)-4-(4-chlorobenzyl)-7-(methoxymethyl)-5,10,16-trimethyltetradecahydro-1H-pyrano[3,4-l][1,4,7,11,14]pentaazacyclooctadecine-2,6,9,12,15(3H)-pentaone;
(4S,7S,10S,14R,16aS,20aS)-14-benzyl-4-(4-chlorobenzyl)-11-(4-(4-(2-((dimethylamino)methyl)-1-methyl-1H-imidazol-5-yl)phenoxy)-2-fluorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-11-(2-(4-(2-(azetidin-1-ylmethyl)-1-methyl-1H-imidazol-5-yl)phenoxy)-4-chlorobenzyl)-14-benzyl-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-2-(azetidin-1-ylmethyl)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-1,4,7,11,14-pentaazacyclooctadecane-3,6,10,15,18-pentaone;
(4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-14-(isoxazol-3-ylmethyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(5-methyl-4-(pyrrolidin-1-ylmethyl)-1H-imidazol-1-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(2-morpholino-2-oxoethyl)hexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(2-(1-hydroxyethyl)-1-methyl-1H-imidazol-5-yl)phenoxy)benzyl)-8-(4-chlorobenzyl)-5-(hydroxymethyl)-2,7,13,14-tetramethyl-1,4,7,11,14-pentaazacyclooctadecane-3,6,10,15,18-pentaone;
(2S,5S,8S,13S,16R)-1-(4-chloro-2-(4-(1-methyl-2-(pyrrolidin-1-ylmethyl)-1H-imidazol-5-yl)phenoxy)benzyl)-8-(4-chlorobenzyl)-16-(2,4-difluorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-11-(2-(4-(2-(2-oxa-6-azaspiro[3.3]heptan-6-ylmethyl)-1-methyl-1H- imidazol-5-yl)phenoxy)-4-ethylbenzyl)-14-benzyl-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5R,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(fluoromethyl)-2,13,14-trimethyl-1,4,7,11,14-pentaazacyclooctadecane-3,6,10,15,18-pentaone;
(2S,5S,8S,13S,16R)-16-benzyl-8-(4-chlorobenzyl)-1-(4-ethyl-2-(4-(1-methyl-2-(pyrrolidin-1-ylmethyl)-1H-imidazol-5-yl)phenoxy)benzyl)-2-(2-fluoroethyl)-5-(methoxymethyl)-7,13,14-trimethyl-1,4,7,11,14-pentaazacyclooctadecane-3,6,10,15,18-pentaone;
(2S,5S,8S,13R,16R)-16-benzyl-1-(4-chloro-2-(4-(2-((dimethylamino)methyl)-1-methyl-1H-imidazol-5-yl)phenoxy)benzyl)-8-(4-chlorobenzyl)-5,13-bis(methoxymethyl)-2,7,14-trimethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(1-methylpyrrolidin-2-yl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-10-(2-(dimethylamino)ethyl)-7-(methoxymethyl)-5,16-dimethylhexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(1-methyl-2-(pent-1-yn-1-yl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-5-(oxetan-3-yl)-1,4,7,11,14-pentaazacyclooctadecane-3,6,10,15,18-pentaone;
(2S,5S,8S,12S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,12,14-tetramethyl-1,4,7,11,14-pentaazacyclooctadecane-3,6,10,15,18-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-16-(2,3,4-trifluorobenzyl)-1,4,7,11,14-pentaazacyclooctadecane-3,6,10,15,18-pentaone;
(2S,5S,8S,13S,16R)-16-benzyl-1-(4-chloro-2-fluoro-6-(4-(1-methyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)-4-(trifluoromethoxy)benzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(5-(4-((2-fluoroethyl)amino)cyclohexyl)pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-2-(2,2,2-trifluoroethyl)-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-2-(2-morpholino-2-oxoethyl)-1,4,7,11,14-pentaazacyclooctadecane-3,6,10,15,18-pentaone;
(4S,7S,10S,16aS,20aS)-11-(4-chloro-2-(4-(2-((dimethylamino)methyl)-1-methyl-1H-imidazol-5-yl)phenoxy)-6-(2,2,2-trifluoroethoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16S)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-16-(pentan-3-yl)-1,4,7,11,14-pentaazacyclooctadecane-3,6,10,15,18-pentaone;
(4S,7S,14R,16aS,20aS)-14-benzyl-4-(4-chlorobenzyl)-11-(2-(4-(2-((dimethylamino)methyl)-1-methyl-1H-imidazol-5-yl)phenoxy)-4,6-difluorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-8-(4-chlorobenzyl)-1-(4-(difluoromethyl)-2-(4-(2-((dimethylamino)methyl)-1-methyl-1H-imidazol-5-yl)phenoxy)benzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-13-(4-(dimethylamino)butyl)-5-(methoxymethyl)-2,7,14-trimethyl-1,4,7,11,14-pentaazacyclooctadecane-3,6,10,15,18-pentaone;
(2S,5S,8S,13S,16R)-16-allyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-16-((5-fluoropyridin-2-yl)methyl)-5-(methoxymethyl)-2,7,13,14-tetramethyl-1,4,7,11,14-pentaazacyclooctadecane-3,6,10,15,18-pentaone;
tert-butyl 3-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)propanoate;
(4S,7S,10S,14R,16aS,20aS)-14-benzyl-4-(4-chlorobenzyl)-11-(2-(4-(2-((dimethylamino)methyl)-1-methyl-1H-imidazol-5-yl)phenoxy)-4-fluorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-1-(2-(4-(2-((bis(methyl-d3)amino)methyl)-1-methyl-1H-imidazol-5-yl)phenoxy)-4-chlorobenzyl)-8-(4-chlorobenzyl)-2-ethyl-5-(methoxymethyl)-7,13,14-trimethyl-1,4,7,11,14-pentaazacyclooctadecane-3,6,10,15,18-pentaone;
(2S,5S,8S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,12,13,14-pentamethyl-1,4,7,11,14-pentaazacyclooctadecane-3,6,10,15,18-pentaone;
(2S,5S,8S,13S,16R)-16-benzyl-1-(4-chloro-2-(3,5-difluoro-4-(1-methyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-10-(2-(3-hydroxy-3-methylazetidin-1-yl)-2-oxoethyl)-7-(methoxymethyl)-5,16-dimethylhexadecahydrobenzo[l][1,4,7,11,14]pentaazacyclooctadecine-2,6,9,12,15(3H)-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(ethoxymethyl)-2,7,13,14,16-pentamethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-2-(2-fluoroethyl)-5-(methoxymethyl)-7,13,14-trimethyl-1,4,7,11,14-pentaazacyclooctadecane-3,6,10,15,18-pentaone;
(2S,5S,8S,13S,16R)-16-benzyl-1-(4-chloro-2-(4-(1,4-dimethyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-16-(3-(difluoromethyl)benzyl)-5-(methoxymethyl)-2,7,13,14-tetramethyl-1,4,7,11,14-pentaazacyclooctadecane-3,6,10,15,18-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-16-(4-(difluoromethyl)benzyl)-5-(methoxymethyl)-2,7,13,14-tetramethyl-1,4,7,11,14-pentaazacyclooctadecane-3,6,10,15,18-pentaone;
(4S,7S,10R,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(2-(((R)-3-fluoropyrrolidin-1-yl)methyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,14R)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyltetradecahydro-1H-pyrano[3,4-l][1,4,7,11,14]pentaazacyclooctadecine-2,6,9,12,15(3H)-pentaone;
(5S,8S,13S,16R)-8-(4-chlorobenzyl)-1-(2-(difluoromethoxy)-6-(4-(2-((dimethylamino)methyl)-1-methyl-1H-imidazol-5-yl)phenoxy)benzyl)-5-(methoxymethyl)-2,7,13,14,16-pentamethyl-1,4,7,11,14-pentaazacyclooctadecane-3,6,10,15,18-pentaone;
(2S,5S,8S,13S)-1-(4-chloro-2-(4-(1-methyl-2-(pyrrolidin-1-ylmethyl)-1H-imidazol-5-yl)phenoxy)benzyl)-8-(4-chlorobenzyl)-16-((1-(difluoromethyl)-1H-pyrazol-3-yl)methyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,14,16-tetramethylhexadecahydrobenzo[l][1,4,7,11,14]pentaazacyclooctadecine-2,6,9,12,15(3H)-pentaone;
(2S,5S,8S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,12,12,14-pentamethyl-1,4,7,11,14-pentaazacyclooctadecane-3,6,10,15,18-pentaone;
(2S,5S,8S,13S)-8-(4-chlorobenzyl)-1-(2-(4-(2-((dimethylamino)methyl)-1-methyl-1H-imidazol-5-yl)phenoxy)benzyl)-5-(hydroxymethyl)-2,7,13,14-tetramethyl-1,4,7,11,14-pentaazacyclooctadecane-3,6,10,15,18-pentaone;
(2S,5S,8S,13S,16R)-1-(4-chloro-2-(4-(1-methyl-2-(pyrrolidin-1-ylmethyl)-1H-imidazol-5-yl)phenoxy)benzyl)-8-(4-chlorobenzyl)-16-(2,3-difluorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-ethoxy-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-methoxypyrimidin-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2-ethyl-5-(methoxymethyl)-7,13,14-trimethyl-1,4,7,11,14-pentaazacyclooctadecane-3,6,10,15,18-pentaone;
(2S,5S,8S,13R,16R)-methyl 16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,14-trimethyl-3,6,10,15,18-pentaoxo-1,4,7,11,14-pentaazacyclooctadecane-13-carboxylate;
(2S,5S,8S,13S,16R)-16-benzyl-1-(4-chloro-2-(3-fluoro-4-(1-methyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(1-(pyrrolidin-1-yl)ethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2R,5S,8S,13S,16R)-16-benzyl-8-(4-chlorobenzyl)-1-(4-(difluoromethyl)-2-(4-(2-((dimethylamino)methyl)-1-methyl-1H-imidazol-5-yl)phenoxy)benzyl)-5-(methoxymethyl)-2,7,13,14-tetramethyl-1,4,7,11,14-pentaazacyclooctadecane-3,6,10,15,18-pentaone;
(2S,5S,8S,13S,16S)-1-(4-chloro-2-(4-(2-((dimethylamino)methyl)-1-methyl-1H-imidazol-5-yl)phenoxy)benzyl)-8-(4-chlorobenzyl)-16-((R)-1-hydroxybutyl)-5-(methoxymethyl)-2,7,13,14-tetramethyl-1,4,7,11,14-pentaazacyclooctadecane-3,6,10,15,18-pentaone;
(2S,5S,8S,13S,16R)-1-(2-(4-(2-(azetidin-1-ylmethyl)-1-methyl-1H-imidazol-5-yl)phenoxy)-4-chlorobenzyl)-16-benzyl-8-(4-chlorobenzyl)-5-(methoxymethyl)-2,7,13,14-tetramethyl-1,4,7,11,14-pentaazacyclooctadecane-3,6,10,15,18-pentaone;
(1S,5S,8S,11S,15R,18R)-15-benzyl-12-(4-chloro-2-(4-(2-((dimethylamino)methyl)-1-methyl-1H-imidazol-5-yl)phenoxy)benzyl)-5-(4-chlorobenzyl)-8-(methoxymethyl)-6,11,17-trimethyl-2,6,9,12,17-pentaazabicyclo[16.1.0]nonadecane-3,7,10,13,16-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,5-bis(methoxymethyl)-7,13,14-trimethyl-1,4,7,11,14-pentaazacyclooctadecane-3,6,10,15,18-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-((difluoromethoxy)methyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-8-(4-chlorobenzyl)-5-(methoxymethyl)-2,7,13,14-tetramethyl-1-(2-(4-(1-methyl-2-(pyrrolidin-1-ylmethyl)-1H-imidazol-5-yl)phenoxy)-4-(trifluoromethyl)benzyl)-1,4,7,11,14-pentaazacyclooctadecane-3,6,10,15,18-pentaone;
(2S,5S,8S,13S,16R)-16-benzyl-1-(4-chloro-2-(4-(1-methyl-2-((E)-pent-1-en-1-yl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
2-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-3,6,10,15,18-pentaoxo-1,4,7,11,14-pentaazacyclooctadecan-2-yl)acetic acid;
(2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-13-(2-fluoroethyl)-5-(methoxymethyl)-2,7,14-trimethyl-1,4,7,11,14-pentaazacyclooctadecane-3,6,10,15,18-pentaone;
(2S,5S,8S,13S,16R)-16-benzyl-1-(4-chloro-2-(4-(5-(1-(2-fluoroethyl)piperidin-4-yl)pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-14-benzyl-4-(4-chlorobenzyl)-11-(2-(4-(2-((R)-1-(dimethylamino)ethyl)-1-methyl-1H-imidazol-5-yl)phenoxy)-4-ethylbenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(2-(methylamino)pyrimidin-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2-(2-hydroxyethyl)-5-(methoxymethyl)-7,13,14-trimethyl-1,4,7,11,14-pentaazacyclooctadecane-3,6,10,15,18-pentaone;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)-6-methylbenzyl)-5-(methoxymethyl)-2,7,13,14-tetramethyl-1,4,7,11,14-pentaazacyclooctadecane-3,6,10,15,18-pentaone;
2-((4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-2,6,9,12,15-pentaoxodocosahydrobenzo[l][1,4,7,11,14]pentaazacyclooctadecin-14-yl)acetonitrile;
(2S,5S,8S,12S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2-(3-(dimethylamino)propyl)-5-(methoxymethyl)-7,12,13,14-tetramethyl-1,4,7,11,14-pentaazacyclooctadecane-3,6,10,15,18-pentaone;
(4S,7S,10R,14R,16aS,20aS)-11-(4-chloro-2-(4-(2-((dimethylamino)methyl)-1-methyl-1H-imidazol-5-yl)phenoxy)benzyl)-4-(4-chlorobenzyl)-10-(2-(dimethylamino)ethyl)-7-(methoxymethyl)-5,16-dimethyl-14-(pyridin-2-ylmethyl)hexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(2-isopropoxy-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(3-morpholino-3-oxopropyl)hexadecahydrobenzo[l][1,4,7,11,14]pentaazacyclooctadecine-2,6,9,12,15(3H)-pentaone;
(2S,5S,8S,12S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,12,13,14-tetramethyl-2-(2-morpholinoethyl)-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-2-(3-oxo-3-(2-oxa-6-azaspiro[3.3]heptan-6-yl)propyl)-1,4,7,11,14-pentaazacyclooctadecane-3,6,10,15,18-pentaone;
(4S,7S,10S,14S,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-(2,3,3-trifluoroallyl)hexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-2-(2,2-difluoroethyl)-5-(methoxymethyl)-7,13,14-trimethyl-1,4,7,11,14-pentaazacyclooctadecane-3,6,10,15,18-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-16-(2,3,5-trifluorobenzyl)-1,4,7,11,14-pentaazacyclooctadecane-3,6,10,15,18-pentaone;
2-(((2S,5S,8S,12S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,12,13,14-pentamethyl-3,6,10,15,18-pentaoxo-1,4,7,11,14-pentaazacyclooctadecan-16-yl)methyl)benzonitrile;
(4S,7S,10S,14R,16aS,20aS)-14-(bicyclo[1.1.1]pentan-1-ylmethyl)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(2-morpholinoethyl)hexadecahydrobenzo[l][1,4,7,11,14]pentaazacyclooctadecine-2,6,9,12,15(3H)-pentaone;
(2S,5S,8S,12S,13S,16R)-16-benzyl-8-(4-chlorobenzyl)-1-(2-(4-(2-((dimethylamino)methyl)-1-methyl-1H-imidazol-5-yl)phenoxy)-4-methylbenzyl)-5-(methoxymethyl)-2,7,12,13,14-pentamethyl-1,4,7,11,14-pentaazacyclooctadecane-3,6,10,15,18-pentaone;
(2S,5S,8S,13S,16R)-1-(4-chloro-2-(4-(1-methyl-2-((S)-1-(pyrrolidin-1-yl)ethyl)-1H-imidazol-5-yl)phenoxy)benzyl)-8-(4-chlorobenzyl)-5-(methoxymethyl)-2,7,13,14,16-pentamethyl-1,4,7,11,14-pentaazacyclooctadecane-3,6,10,15,18-pentaone;
(4S,7S,10S,14R,16aS,20aS)-14-benzyl-11-(4-chloro-2-(4-(2-((R)-1-(dimethylamino)ethyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-11-(4-chloro-2-(4-(4-((dimethylamino)methyl)-5-methyl-1H-imidazol-1-yl)phenoxy)benzyl)-4-(4-chlorobenzyl)-7-(methoxymethyl)-5,10,16-trimethyl-14-(pyridin-2-ylmethyl)hexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(2-oxo-2-(2-oxa-6-azaspiro[3.3]heptan-6-yl)ethyl)hexadecahydrobenzo[l][1,4,7,11,14]pentaazacyclooctadecine-2,6,9,12,15(3H)-pentaone;
(2S,5S,8S,12S,13S,16R)-16-allyl-8-(4-chlorobenzyl)-1-(2-(4-(2-((dimethylamino)methyl)-1-methyl-1H-imidazol-5-yl)phenoxy)-4-methylbenzyl)-5-(methoxymethyl)-2,7,12,13,14-pentamethyl-1,4,7,11,14-pentaazacyclooctadecane-3,6,10,15,18-pentaone;
(2S,5S,8S,13S,16R)-1-(4-chloro-2-(4-(1-methyl-2-(pyrrolidin-1-ylmethyl)-1H-imidazol-5-yl)phenoxy)benzyl)-8-(4-chlorobenzyl)-16-(4-fluorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-10-(2-aminoethyl)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethylhexadecahydrobenzo[l][1,4,7,11,14]pentaazacyclooctadecine-2,6,9,12,15(3H)-pentaone;
(4S,7S,10S,14R,16aS,20aS)-14-allyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
2-(((4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxo-10-(3-(pyrrolidin-1-yl)propyl)docosahydrobenzo[l][1,4,7,11,14]pentaazacyclooctadecin-14-yl)methyl)pyridine 1-oxide;
(2S,5S,8S,13S,16R)-1-(2-(4-(4-(2-oxa-6-azaspiro[3.3]heptan-6-ylmethyl)-5-methyl-1H-imidazol-1-yl)phenoxy)-4-chlorobenzyl)-16-benzyl-8-(4-chlorobenzyl)-5-(methoxymethyl)-2,7,13,14-tetramethyl-1,4,7,11,14-pentaazacyclooctadecane-3,6,10,15,18-pentaone;
3-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)-N-(piperidin-4-yl)benzamide;
(2S,5S,8S,13S,16R)-16-benzyl-1-(4-chloro-2-(4-(5,6,7,8-tetrahydro-[1,2,4]triazolo[4,3-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(5,6,7,8-tetrahydro-1,6-naphthyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(7-(2-methoxyacetyl)-5,6,7,8-tetrahydro-[1,2,4]triazolo[4,3-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(6-(2-methoxyacetyl)-5,6,7,8-tetrahydro-1,6-naphthyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
3-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)-N-(1-methylpiperidin-4-yl)benzamide;
(2S,5S,8S,13S,16R)-16-benzyl-1-(4-chloro-2-(4-(5-(pyrrolidin-2-yl)pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(4-((diethylamino)methyl)-1H-1,2,3-triazol-1-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
5-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-N-(piperidin-4-yl)picolinamide;
(2S,5S,8S,13S,16R)-16-benzyl-1-(4-chloro-2-(4-(7-isopropyl-5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(7-(pyrrolidine-3-carbonyl)-5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-((3-oxo-2-(piperidin-4-yl)isoindolin-5-yl)oxy)benzyl)-8-(4-chlorobenzyl)-5-(hydroxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(4-(pyrrolidin-1-ylmethyl)-1H-1,2,3-triazol-1-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(4-(piperidin-1-ylmethyl)-1H-1,2,3-triazol-1-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(7-((R)-pyrrolidine-3-carbonyl)-5,6,7,8-tetrahydro-[1,2,4]triazolo[4,3-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-((2-(1-methylpiperidin-4-yl)-3-oxoisoindolin-5-yl)oxy)benzyl)-8-(4-chlorobenzyl)-5-(hydroxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(7-((S)-pyrrolidine-3-carbonyl)-5,6,7,8-tetrahydro-[1,2,4]triazolo[4,3-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(7-isobutyryl-5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(7-(tetrahydrofuran-3-carbonyl)-5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(4-((dimethylamino)methyl)-5-methyl-1H-1,2,3-triazol-1-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(5-(1,4-dimethylpiperazin-2-yl)pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1′,2′,3′,6′-tetrahydro-[3,4′-bipyridin]-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(5-(piperidin-4-yl)pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-((2-(1-(cyclopropylmethyl)piperidin-4-yl)-3-oxoisoindolin-5-yl)oxy)benzyl)-8-(4-chlorobenzyl)-5-(hydroxymethyl)-2,7,13,14-tetramethyl-1,4,7,11,14-pentaazacyclooctadecane-3,6,10,15,18-pentaone;
(2S,5S,8S)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,14-trimethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(hydroxymethyl)-2,7,13,14-tetramethyl-1,4,7,11,14-pentaazacyclooctadecane-3,6,10,15,18-pentaone;
(2S,5S,8S,13S,16R)-16-benzyl-8-(4-chlorobenzyl)-5-(hydroxymethyl)-2,7,13,14-tetramethyl-1-(2-(2-oxo-2-(5H-pyrrolo[3,4-b]pyridin-6(7H)-yl)ethoxy)benzyl)-1,4,7,11,14-pentaazacyclooctadecane-3,6,10,15,18-pentaone;
(6R,10S,13S,16S)-6-benzyl-9-(4-chloro-2-(4-(2-((dimethylamino)methyl)-1-methyl-1H-imidazol-5-yl)phenoxy)benzyl)-16-(4-chlorobenzyl)-13-(methoxymethyl)-4,10,15-trimethyl-4,9,12,15,19-pentaazaspiro[2.17]icosane-5,8,11,14,18-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(4-methyl-5-(pyrrolidin-1-ylmethyl)pyridin-3-yl)phenoxy)benzyl)-8-(4-chlorobenzyl)-5-(hydroxymethyl)-2,7,13,14-tetramethyl-1,4,7,11,14-pentaazacyclooctadecane-3,6,10,15,18-pentaone;
(1R,5S,8S,11S,15R,18S)-15-benzyl-12-(4-chloro-2-(4-(2-((dimethylamino)methyl)-1-methyl-1H-imidazol-5-yl)phenoxy)benzyl)-5-(4-chlorobenzyl)-8-(methoxymethyl)-6,11,17-trimethyl-2,6,9,12,17-pentaazabicyclo[16.1.0]nonadecane-3,7,10,13,16-pentaone;
(2S,5S,8S,13S,16R)-5-((1H-tetrazol-5-yl)methyl)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-1,4,7,11,14-pentaazacyclooctadecane-3,6,10,15,18-pentaone;
ethyl 1-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-5-methyl-1H-imidazole-4-carboxylate;
(2S,5S,8S,13S,16R)-16-benzyl-8-(4-chlorobenzyl)-1-(4-ethyl-2-(4-(1-methyl-2-(pyrrolidin-1-ylmethyl)-1H-imidazol-5-yl)phenoxy)benzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-(1-hydroxypropyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-14-benzyl-11-(4-chloro-2-(4-(1-methyl-2-((R)-1-(pyrrolidin-1-yl)ethyl)-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)-6-methoxybenzyl)-5-(methoxymethyl)-2,7,13,14-tetramethyl-1,4,7,11,14-pentaazacyclooctadecane-3,6,10,15,18-pentaone;
(2S,5S,8S,13S,16R)-1-(4-chloro-2-(4-(1-methyl-2-(pyrrolidin-1-ylmethyl)-1H-imidazol-5-yl)phenoxy)benzyl)-8-(4-chlorobenzyl)-16-(3-fluorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2-(2-methoxyethyl)-5-(methoxymethyl)-7,13,14-trimethyl-1,4,7,11,14-pentaazacyclooctadecane-3,6,10,15,18-pentaone;
(4S,7S,10S,14R,16aS,20aS)-11-(2-(4-(2-(2-oxa-6-azaspiro[3.3]heptan-6-ylmethyl)-1-methyl-1H- imidazol-5-yl)phenoxy)-4-chlorobenzyl)-14-benzyl-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-2-(3-morpholinopropyl)-1,4,7,11,14-pentaazacyclooctadecane-3,6,10,15,18-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-16-methoxy-5-(methoxymethyl)-2,7,13,14-tetramethyl-1,4,7,11,14-pentaazacyclooctadecane-3,6,10,15,18-pentaone;
2-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-3,6,10,15,18-pentaoxo-1,4,7,11,14-pentaazacyclooctadecan-5-yl)-N,N-dimethylacetamide;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14,16-pentamethyl-1,4,7,11,14-pentaazacyclooctadecane-3,6,10,15,18-pentaone;
(2S,5S,8S,13S,16R)-16-benzyl-1-(4-chloro-2-(4-(7-((S)-tetrahydrofuran-3-carbonyl)-5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
1-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-N-isopropyl-5-methyl-1H-imidazole-4-carboxamide (4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(hydroxymethyl)-5,10,14,16-tetramethylhexadecahydrobenzo[l][1,4,7,11,14]pentaazacyclooctadecine-2,6,9,12,15(3H)-pentaone;
(2S,5S,8S,13S,16R)-1-(4-chloro-2-(4-(1-methyl-2-(pyrrolidin-1-ylmethyl)-1H-imidazol-5-yl)phenoxy)benzyl)-8-(4-chlorobenzyl)-16-(3,4-difluorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)-3,6-difluorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-(1-hydroxyethyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-8-(4-chlorobenzyl)-1-((4-(4-(2-((dimethylamino)methyl)-1-methyl-1H-imidazol-5-yl)phenoxy)-6-oxo-1,6-dihydropyridin-3-yl)methyl)-5-(methoxymethyl)-2,7,13,14-tetramethyl-1,4,7,11,14-pentaazacyclooctadecane-3,6,10,15,18-pentaone;
(2S,5S,8S,13S,16R)-13-(4-aminobutyl)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,14-trimethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-((difluoromethoxy)methyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(7-((R)-tetrahydrofuran-3-carbonyl)-5,6,7,8-tetrahydroimidazo[1,2-a]pyrazin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
2-(((4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-2,6,9,12,15-pentaoxodocosahydrobenzo[l][1,4,7,11,14]pentaazacyclooctadecin-14-yl)methyl)pyridine 1-oxide;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-2-ethyl-5-(methoxymethyl)-7,13,14,16-tetramethyl-1,4,7,11,14-pentaazacyclooctadecane-3,6,10,15,18-pentaone;
tert-butyl 2-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-3,6,10,15,18-pentaoxo-1,4,7,11,14-pentaazacyclooctadecan-2-yl)acetate;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-2-(3-morpholino-3-oxopropyl)-1,4,7,11,14-pentaazacyclooctadecane-3,6,10,15,18-pentaone;
(2S,5S,8S,13S,16R)-16-benzyl-1-(4-chloro-2-(4-(6-((S)-pyrrolidine-3-carbonyl)-5,6,7,8-tetrahydro-1,6-naphthyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13R,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,14-trimethyl-3,6,10,15,18-pentaoxo-1,4,7,11,14-pentaazacyclooctadecane-13-carboxylic acid;
(2S,5S,8S,13S,16R)-16-benzyl-1-(4-chloro-2-(4-(1-methyl-2-(1-(pyrrolidin-1-yl)ethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-8-(4-chlorobenzyl)-1-(4-(difluoromethoxy)-2-(4-(2-((dimethylamino)methyl)-1-methyl-1H-imidazol-5-yl)phenoxy)benzyl)-5-(methoxymethyl)-2,7,13,14-tetramethyl-1,4,7,11,14-pentaazacyclooctadecane-3,6,10,15,18-pentaone;
2-(((2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-3,6,10,15,18-pentaoxo-1,4,7,11,14-pentaazacyclooctadecan-16-yl)methyl)-5-fluoropyridine 1-oxide;
(2S,5S,8S,13S,16R)-16-benzyl-1-(4-chloro-2-(4-(2-ethyl-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
tert-butyl (3-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)propyl)carbamate;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-16-(cyclohexylmethyl)-5-(methoxymethyl)-2,7,13,14-tetramethyl-1,4,7,11,14-pentaazacyclooctadecane-3,6,10,15,18-pentaone;
(5S,8S,13S,16R)-1-(4-chloro-2-(4-(2-((dimethylamino)methyl)-1-methyl-1H-imidazol-5-yl)phenoxy)benzyl)-8-(4-chlorobenzyl)-13-isobutyl-5-(methoxymethyl)-2,7,14,16-tetramethyl-1,4,7,11,14-pentaazacyclooctadecane-3,6,10,15,18-pentaone;
tert-butyl 2-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-3,6,10,15,18-pentaoxo-1,4,7,11,14-pentaazacyclooctadecan-5-yl)acetate;
(2S,5S,8S,12S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,12,13,14-pentamethyl-1,4,7,11,14-pentaazacyclooctadecane-3,6,10,15,18-pentaone;
(4S,7S,10S,14R,16aS,20aR)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyltetradecahydro-1H-pyrano[3,4-l][1,4,7,11,14]pentaazacyclooctadecine-2,6,9,12,15(3H)-pentaone;
1-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-N-(2-hydroxyethyl)-5-methyl-1H-imidazole-4-carboxamide;
(2S,5S,8S,13S,16R)-1-(2-(4-(2-(2-oxa-6-azaspiro[3.3]heptan-6-ylmethyl)-1-methyl-1H-imidazol-5-yl)phenoxy)-4-chlorobenzyl)-16-benzyl-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-13-(2-(dimethylamino)ethyl)-5-(methoxymethyl)-2,7,14-trimethyl-1,4,7,11,14-pentaazacyclooctadecane-3,6,10,15,18-pentaone;
tert-butyl (3-((4S,7S,10S,14R,16aS,20aS)-14-benzyl-11-(4-chloro-2-(4-(1-methyl-2-(pyrrolidin-1-ylmethyl)-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)propyl)carbamate;
(2S,5S,8S,13S,16R)-2-(3-aminopropyl)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-1,4,7,11,14-pentaazacyclooctadecane-3,6,10,15,18-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(hydroxymethyl)-2,7,13,14,16-pentamethyl-1,4,7,11,14-pentaazacyclooctadecane-3,6,10,15,18-pentaone;
(4S,7S,10R,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(hydroxymethyl)-5,10,16-trimethylhexadecahydrobenzo[l][1,4,7,11,14]pentaazacyclooctadecine-2,6,9,12,15(3H)-pentaone;
(2S,5S,8S,13S)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,16aS,20aS)-4-(4-chlorobenzyl)-11-(2-(difluoromethoxy)-6-(4-(2-((dimethylamino)methyl)-1-methyl-1H-imidazol-5-yl)phenoxy)benzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(2-methoxy-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,12S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,12,13,14-pentamethyl-1,4,7,11,14-pentaazacyclooctadecane-3,6,10,15,18-pentaone;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)-4-fluorobenzyl)-5-(methoxymethyl)-2,7,13,14-tetramethyl-1,4,7,11,14-pentaazacyclooctadecane-3,6,10,15,18-pentaone;
(2S,5S,8S,13S)-8-(4-chlorobenzyl)-1-(2-(4-(2-((dimethylamino)methyl)-1-methyl-1H-imidazol-5-yl)phenoxy)benzyl)-5-(hydroxymethyl)-2-(methoxymethyl)-7,13,14-trimethyl-1,4,7,11,14-pentaazacyclooctadecane-3,6,10,15,18-pentaone;
(6S,11S,14S,17S)-18-(4-chloro-2-(4-(2-((dimethylamino)methyl)-1-methyl-1H-imidazol-5-yl)phenoxy)benzyl)-11-(4-chlorobenzyl)-14-(methoxymethyl)-5,6,12,17-tetramethyl-5,8,12,15,18-pentaazaspiro[2.17]icosane-4,9,13,16,19-pentaone;
(4S,7S,10S,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(1-methyl-2-(pyrrolidin-1-ylmethyl)-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10R,14R,16aS,20aS)-10-(2-aminoethyl)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethylhexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(1-methyl-2-(morpholinomethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-16-(3-(trifluoromethoxy)benzyl)-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2-ethyl-5-(hydroxymethyl)-7,13,14-trimethyl-1,4,7,11,14-pentaazacyclooctadecane-3,6,10,15,18-pentaone;
(4S,7S,10S,14R,16aS,20aS)-14-benzyl-4-(4-chlorobenzyl)-11-(2-(4-(2-((dimethylamino)methyl)-1-methyl-1H-imidazol-5-yl)phenoxy)-4,6-difluorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chloro-3-fluorobenzyl)-2-ethyl-5-(methoxymethyl)-7,13,14-trimethyl-1,4,7,11,14-pentaazacyclooctadecane-3,6,10,15,18-pentaone;
(2S,5S,8S,13S,16R)-16-benzyl-8-(4-chlorobenzyl)-1-((2-(4-(2-((dimethylamino)methyl)-1-methyl-1H-imidazol-5-yl)phenoxy)pyridin-3-yl)methyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,13,14-trimethyl-2-(2-(methylsulfonyl)ethyl)-1,4,7,11,14-pentaazacyclooctadecane-3,6,10,15,18-pentaone;
(2S,5S,8S,13S,16R)-16-benzyl-1-(4-chloro-2-(4-(6-((R)-pyrrolidine-3-carbonyl)-5,6,7,8-tetrahydro-1,6-naphthyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
2-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-3,6,10,15,18-pentaoxo-1,4,7,11,14-pentaazacyclooctadecan-5-yl)acetic acid;
(2S,5S,8S,13S,16R)-16-benzyl-8-(4-chlorobenzyl)-1-((3-(4-(2-((dimethylamino)methyl)-1-methyl-1H-imidazol-5-yl)phenoxy)pyridin-2-yl)methyl)-5-(methoxymethyl)-2,7,13,14-tetramethyl-1,4,7,11,14-pentaazacyclooctadecane-3,6,10,15,18-pentaone;
(2S,5S,8S,13S)-1-(4-chloro-2-(4-(2-((dimethylamino)methyl)-1-methyl-1H-imidazol-5-yl)phenoxy)benzyl)-8-(4-chlorobenzyl)-16-cyclopropyl-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(1-(pyrrolidin-1-yl)ethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
N-(4-((2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,14-trimethyl-3,6,10,15,18-pentaoxo-1,4,7,11,14-pentaazacyclooctadecan-13-yl)butyl)acetamide;
2-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)-N,N-bis(2-methoxyethyl)acetamide;
2-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)acetic acid;
(2S,5S,8S,13S,16R)-16-benzyl-1-(4-chloro-2-(4-(2-(((S)-3-hydroxypyrrolidin-1-yl)methyl)-1-methyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
2-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-3,6,10,15,18-pentaoxo-1,4,7,11,14-pentaazacyclooctadecan-5-yl)-N-methylacetamide;
(2R,5S,8S,13S,16R)-16-benzyl-1-(4-chloro-2-(4-(2-(hydroxymethyl)-1-methyl-1H-imidazol-5-yl)phenoxy)benzyl)-8-(4-chlorobenzyl)-2,5-bis(methoxymethyl)-7,13,14-trimethyl-1,4,7,11,14-pentaazacyclooctadecane-3,6,10,15,18-pentaone;
(4S,7S,10S,14R,16aS,20aS)-14-benzyl-4-(4-chlorobenzyl)-11-(4-ethyl-2-(4-(1-methyl-2-(pyrrolidin-1-ylmethyl)-1H-imidazol-5-yl)phenoxy)benzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aR,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyltetradecahydro-1H-pyrano[3,4-l][1,4,7,11,14]pentaazacyclooctadecine-2,6,9,12,15(3H)-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2-(4-(dimethylamino)butyl)-5-(methoxymethyl)-7,13,14-trimethyl-1,4,7,11,14-pentaazacyclooctadecane-3,6,10,15,18-pentaone;
((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-5,10,16-trimethyl-2,6,9,12,15-pentaoxodocosahydrobenzo[l][1,4,7,11,14]pentaazacyclooctadecin-7-yl)methyl pivalate;
(5S,8S,11S,16S)-4-(4-chloro-2-(4-(2-((dimethylamino)methyl)-1-methyl-1H-imidazol-5-yl)phenoxy)benzyl)-11-(4-chlorobenzyl)-8-(methoxymethyl)-5,10,17-trimethyl-1,4,7,10,14,17-hexaazabicyclo[14.2.1]nonadecane-3,6,9,13,18-pentaone;
(4S,7S,14R,16aS,20aS)-14-benzyl-4-(4-chlorobenzyl)-11-(2-(4-(2-((dimethylamino)methyl)-1-methyl-1H-imidazol-5-yl)phenoxy)-4-fluorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,13S,16R)-1-(4-chloro-2-(4-(1-methyl-2-(pyrrolidin-1-ylmethyl)-1H-imidazol-5-yl)phenoxy)benzyl)-8-(4-chlorobenzyl)-16-(2-fluorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-1-(2-chloro-6-(4-(2-((dimethylamino)methyl)-1-methyl-1H-imidazol-5-yl)phenoxy)benzyl)-8-(4-chlorobenzyl)-5-(methoxymethyl)-2,7,13,14,16-pentamethyl-1,4,7,11,14-pentaazacyclooctadecane-3,6,10,15,18-pentaone;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)-3-fluorobenzyl)-5-(methoxymethyl)-2,7,13,14-tetramethyl-1,4,7,11,14-pentaazacyclooctadecane-3,6,10,15,18-pentaone;
3-((2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-2,7,13,14-tetramethyl-3,6,10,15,18-pentaoxo-1,4,7,11,14-pentaazacyclooctadecan-5-yl)propanoic acid;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7,10-bis(methoxymethyl)-5,16-dimethylhexadecahydrobenzo[l][1,4,7,11,14]pentaazacyclooctadecine-2,6,9,12,15(3H)-pentaone;
(2S,5S,8S,13R,16R)-16-benzyl-1-(4-chloro-2-(4-(2-((dimethylamino)methyl)-1-methyl-1H-imidazol-5-yl)phenoxy)benzyl)-8-(4-chlorobenzyl)-13-(hydroxymethyl)-5-(methoxymethyl)-2,7,14-trimethyl-1,4,7,11,14-pentaazacyclooctadecane-3,6,10,15,18-pentaone;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)-4,6-difluorobenzyl)-5-(methoxymethyl)-2,7,13,14-tetramethyl-1,4,7,11,14-pentaazacyclooctadecane-3,6,10,15,18-pentaone;
tert-butyl 2-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)acetate;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-16-(3-(difluoromethoxy)benzyl)-5-(methoxymethyl)-2,7,13,14-tetramethyl-1,4,7,11,14-pentaazacyclooctadecane-3,6,10,15,18-pentaone;
2-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)-N-(2-methoxyethyl)-N-methylacetamide;
(2S,5S,8S,13S,16R)-16-benzyl-8-(4-chlorobenzyl)-1-(2-(4-(2-((dimethylamino)methyl)-1-methyl-1H-imidazol-5-yl)phenoxy)-4-methylbenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-14-benzyl-11-(4-chloro-2-(4-(2-((S)-1-(dimethylamino)ethyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-(1-hydroxyethyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(hydroxymethyl)-5,10,16-trimethylhexadecahydrobenzo[l][1,4,7,11,14]pentaazacyclooctadecine-2,6,9,12,15(3H)-pentaone;
1-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-N-ethyl-5-methyl-1H-imidazole-4-carboxamide;
3-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)propanoic acid;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-16-(2,3,5,6-tetrafluorobenzyl)-1,4,7,11,14-pentaazacyclooctadecane-3,6,10,15,18-pentaone;
(1R,5S,8S,11S,15R,18S)-15-benzyl-12-(4-chloro-2-(4-(2-((dimethylamino)methyl)-1-methyl-1H-imidazol-5-yl)phenoxy)benzyl)-5-(4-chlorobenzyl)-8-(methoxymethyl)-6,11,17-trimethyl-2,6,9,12,17-pentaazabicyclo[16.1.0]nonadecane-3,7,10,13,16-pentaone;
(2S,5S,8S,13S,16R)-16-benzyl-1-(4-chloro-2-(4-(2-((dimethylamino)methyl)-1-methyl-1H-imidazol-5-yl)phenoxy)benzyl)-8-(4-chloro-2-fluorobenzyl)-2-ethyl-5-(methoxymethyl)-7,13,14-trimethyl-1,4,7,11,14-pentaazacyclooctadecane-3,6,10,15,18-pentaone;
3-(4-(2-(((2S,5S,8S,13S,16R)-16-benzyl-8-(4-chlorobenzyl)-5-(methoxymethyl)-2,7,13,14-tetramethyl-3,6,10,15,18-pentaoxo-1,4,7,11,14-pentaazacyclooctadecan-1-yl)methyl)-5-chlorophenoxy)phenyl)-6,8-dihydro-5H-spiro[imidazo[1,2-a]pyrazine-7,1′-pyrrolidin]-1′-ium chloride;
(4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-10-(2-(dimethylamino)ethyl)-7-(methoxymethyl)-5,16-dimethyl-14-(pyridin-2-ylmethyl)hexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(3-(pyrrolidin-1-yl)propyl)hexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(1-methyl-2-(pyrrolidin-1-ylmethyl)-1H-imidazol-5-yl)phenoxy)benzyl)-4-(4-chlorobenzyl)-7-(methoxymethyl)-5,16-dimethyl-10-(3-morpholinopropyl)hexadecahydrobenzo[l][1,4,7,11,14]pentaazacyclooctadecine-2,6,9,12,15(3H)-pentaone;
(4S,7S,10S,16aS,20aS)-14-((1H-pyrazol-1-yl)methyl)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(3-(3,3,4,4-tetrafluoropyrrolidin-1-yl)propyl)hexadecahydrobenzo[l][1,4,7,11,14]pentaazacyclooctadecine-2,6,9,12,15(3H)-pentaone;
(5S,8S,13S,16R)-1-(4-chloro-2-(4-(2-((dimethylamino)methyl)-1-methyl-1H-imidazol-5-yl)phenoxy)benzyl)-8-(4-chlorobenzyl)-13-isobutyl-5-(methoxymethyl)-2,7,14,16-tetramethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,13,14-trimethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-(pyrrolidin-1-ylmethyl)-1H-imidazol-5-yl)phenoxy)benzyl)-8-(4-chlorobenzyl)-5-(2-fluoroethyl)-2,7,13,14-tetramethyl-1,4,7,11,14-pentaazacyclooctadecane-3,6,10,15,18-pentaone;
(2S,5S,8S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,13,14-tetramethyl-16-(2,3,6-trifluorobenzyl)-1,4,7,11,14-pentaazacyclooctadecane-3,6,10,15,18-pentaone;
(2S,5S,8S,13S,16R)-16-benzyl-1-(4-chloro-2-(4-(5-methyl-4-(pyrrolidin-1-ylmethyl)-1H-imidazol-1-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-14-benzyl-11-(4-chloro-2-(4-(5-methyl-4-(pyrrolidin-1-ylmethyl)-1H-imidazol-1-yl)phenoxy)benzyl)-4-(4-chlorobenzyl)-7-(hydroxymethyl)-5,10,16-trimethylhexadecahydrobenzo[l][1,4,7,11,14]pentaazacyclooctadecine-2,6,9,12,15(3H)-pentaone;
(2S,5S,8S,12S,13S,16R)-16-benzyl-1-(4-chloro-2-(4-(2-((R)-1-(dimethylamino)ethyl)-1-methyl-1H-imidazol-5-yl)phenoxy)benzyl)-8-(4-chlorobenzyl)-5-(methoxymethyl)-2,7,12,13,14-pentamethyl-1,4,7,11,14-pentaazacyclooctadecane-3,6,10,15,18-pentaone;
(1S,6S,9S,12S,16S)-13-(4-chloro-2-(4-(2-((dimethylamino)methyl)-1-methyl-1H-imidazol-5-yl)phenoxy)benzyl)-6-(4-chlorobenzyl)-9-(methoxymethyl)-7,12,17,19-tetramethyl-3,7,10,13,17,19-hexaazabicyclo[14.2.2]icosane-4,8,11,14,18,20-hexaone;
(4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-propylhexadecahydrobenzo[l][1,4,7,11,14]pentaazacyclooctadecine-2,6,9,12,15(3H)-pentaone;
(4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-((6-methylpyridin-2-yl)methyl)hexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(5-methyl-4-(morpholinomethyl)-1H-imidazol-1-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
diethyl 4,4′-((3-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)propyl)azanediyl)dibutanoate;
(4S,7S,10S,14R,16aS,20aS)-14-benzyl-11-(4-chloro-2-(4-(2-((R)-1-(dimethylamino)propyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
2-((4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-2,6,9,12,15-pentaoxodocosahydrobenzo[l][1,4,7,11,14]pentaazacyclooctadecin-10-yl)-N,N-dimethylacetamide;
(2S,5S,8S,12S,13S,16R)-16-benzyl-1-(4-chloro-2-(4-(4-((R)-1-(dimethylamino)ethyl)-5-methyl-1H-imidazol-1-yl)phenoxy)benzyl)-8-(4-chlorobenzyl)-5-(methoxymethyl)-2,7,12,13,14-pentamethyl-1,4,7,11,14-pentaazacyclooctadecane-3,6,10,15,18-pentaone;
(2S,5S,8S,13S,16R)-16-benzyl-1-(4-chloro-2-(4-(2-(3,6-dihydro-2H-pyran-4-yl)-1-methyl-1H- imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(3-morpholinopropyl)hexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-((5-(2-((dimethylamino)methyl)-1-methyl-1H-imidazol-5-yl)pyridin-2-yl)oxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aR)-14-benzyl-11-(4-chloro-2-(4-(1-methyl-2-(pyrrolidin-1-ylmethyl)-1H-imidazol-5-yl)phenoxy)benzyl)-4-(4-chlorobenzyl)-7-(methoxymethyl)-5,10,16-trimethyltetradecahydro-1H-pyrano[3,4-l][1,4,7,11,14]pentaazacyclooctadecine-2,6,9,12,15(3H)-pentaone;
(4S,7S,10S,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-((2-oxopyridin-1(2H)-yl)methyl)hexadecahydrobenzo[l][1,4,7,11,14]pentaazacyclooctadecine-2,6,9,12,15(3H)-pentaone;
(4S,7S,10S,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-((2-oxopyridin-1(2H)-yl)methyl)hexadecahydrobenzo[l][1,4,7,11,14]pentaazacyclooctadecine-2,6,9,12,15(3H)-pentaone;
(4S,7S,10S,14R,16aS,20aS)-14-allyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(3-morpholinopropyl)hexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(4-((dimethylamino)methyl)-5-methyl-1H-imidazol-1-yl)phenoxy)benzyl)-4-(4-chlorobenzyl)-7-(hydroxymethyl)-5,10,16-trimethylhexadecahydrobenzo[l][1,4,7,11,14]pentaazacyclooctadecine-2,6,9,12,15(3H)-pentaone;
(2S,5S,8S,12S,13S,16R)-1-(4-chloro-2-(4-(1-methyl-2-(pyrrolidin-1-ylmethyl)-1H-imidazol-5-yl)phenoxy)benzyl)-8-(4-chlorobenzyl)-5-(methoxymethyl)-2,7,12,13,14-pentamethyl-16-(2,3,4-trifluorobenzyl)-1,4,7,11,14-pentaazacyclooctadecane-3,6,10,15,18-pentaone;
(4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-(pyridin-3-ylmethyl)hexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(3-methyl-4-(1-methyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1-methyl-2-vinyl-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,12S,13S,16R)-16-benzyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-7,12,13,14-tetramethyl-2-(3-morpholinopropyl)-1,4,7,11,14-pentaazacyclooctadecane-3,6,10,15,18-pentaone;
(4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyl-14-(pyridin-2-ylmethyl)hexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(1-methyl-2-(pyrrolidin-1-ylmethyl)-1H-imidazol-5-yl)phenoxy)benzyl)-4-(4-chlorobenzyl)-10-(3-(dimethylamino)propyl)-7-(methoxymethyl)-5,16-dimethylhexadecahydrobenzo[l][1,4,7,11,14]pentaazacyclooctadecine-2,6,9,12,15(3H)-pentaone;
(4S,7S,10S,14R,16aS,20aS)-14-(but-2-en-1-yl)-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
tert-butyl 3-((4S,7S,10R,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(hydroxymethyl)-5,16-dimethyl-2,6,9,12,15-pentaoxodocosahydrobenzo[l][1,4,7,11,14]pentaazacyclooctadecin-10-yl)propanoate;
(4S,7S,10R,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-10-(2-(dimethylamino)ethyl)-7-(methoxymethyl)-5,16-dimethylhexadecahydrobenzo[l][1,4,7,11,14]pentaazacyclooctadecine-2,6,9,12,15(3H)-pentaone;
(2S,5S,8S,12S,13S,16R)-16-allyl-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,12,13,14-pentamethyl-1,4,7,11,14-pentaazacyclooctadecane-3,6,10,15,18-pentaone;
(4S,7S,10S,14R,16aS,20aS)-11-(4-chloro-2-(4-(2-((dimethylamino)methyl)-1-methyl-1H-imidazol-5-yl)phenoxy)benzyl)-4-(4-chlorobenzyl)-14-(cyclopropylmethyl)-7-(methoxymethyl)-5,10,16-trimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-10-(3-(dimethylamino)propyl)-7-(methoxymethyl)-5,16-dimethylhexadecahydrobenzo[l][1,4,7,11,14]pentaazacyclooctadecine-2,6,9,12,15(3H)-pentaone;
(4S,7S,10S,14R,16aS,20aS)-14-benzyl-11-(4-chloro-2-(4-(4-((dimethylamino)methyl)-5-methyl-1H-imidazol-1-yl)phenoxy)benzyl)-4-(4-chlorobenzyl)-7-(methoxymethyl)-5,10,16-trimethylhexadecahydrobenzo[l][1,4,7,11,14]pentaazacyclooctadecine-2,6,9,12,15(3H)-pentaone;
(2S,5S,8S,12S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,12,13,14-pentamethyl-16-(2,3,4-trifluorobenzyl)-1,4,7,11,14-pentaazacyclooctadecane-3,6,10,15,18-pentaone;
(4S,7S,10R,14R,16aS,20aR)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,10,16-trimethyltetradecahydro-1H-pyrano[3,4-l][1,4,7,11,14]pentaazacyclooctadecine-2,6,9,12,15(3H)-pentaone;
(2S,5S,8S,12S,13S,16R)-1-(4-chloro-2-(4-(2-((dimethylamino)methyl)-1-methyl-1H-imidazol-5-yl)phenoxy)benzyl)-8-(4-chlorobenzyl)-5-(methoxymethyl)-2,7,12,13,14-pentamethyl-16-(pyridin-2-ylmethyl)-1,4,7,11,14-pentaazacyclooctadecane-3,6,10,15,18-pentaone;
(4S,7S,10S,14R,16aS,20aS)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethyl-10-(3-(2-oxopyrrolidin-1-yl)propyl)hexadecahydrobenzo[l][1,4,7,11,14]pentaazacyclooctadecine-2,6,9,12,15(3H)-pentaone;
(2S,5S,8S,13S,16R)-16-benzyl-1-(4-chloro-2-(2,6-difluoro-4-(1-methyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(2,3-difluoro-4-(1-methyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1-ethyl-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(4-methyl-5-(pyrrolidin-1-ylmethyl)pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(8-(pyrrolidin-1-ylmethyl)imidazo[1,2-a]pyridin-3-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone;
(2S,5S,8S,13S,16R)-16-benzyl-1-(4-chloro-2-(4-(1-(2-fluoroethyl)-2-(pyrrolidin-1-ylmethyl)-1H-imidazol-5-yl)phenoxy)benzyl)-8-(4-chlorobenzyl)-5-(methoxymethyl)-2,7,13,14-tetramethyl-1,4,7,11,14-pentaazacyclooctadecane-3,6,10,15,18-pentaone; and
(4S,7S,10S,14R,16aS,20aS)-10-(3-aminopropyl)-14-benzyl-11-(4-chloro-2-(4-(2-((dimethylamino)methyl)-1-methyl-1H-imidazol-5-yl)phenoxy)benzyl)-4-(4-chlorobenzyl)-7-(methoxymethyl)-5,16-dimethylhexadecahydrobenzo[l][1,4,7,11,14]pentaazacyclooctadecine-2,6,9,12,15(3H)-pentaone;
or a pharmaceutically acceptable salt, hydrate, solvate, stereoisomer, or tautomer thereof.

28. (canceled)

29. A pharmaceutical composition comprising a compound according to claim 1, or a pharmaceutically acceptable salt thereof, and one or more pharmaceutically acceptable carriers or excipients.

30-35. (canceled)

36. A method for treating, preventing, ameliorating or delaying the progression of a PCSK9-mediated disease or disorder comprising the step of administering to a patient in need thereof a therapeutically effective amount of a compound according to claim 1, or a pharmaceutically acceptable salt thereof.

37. The method of claim 36, wherein said PCSK9-mediated disease or disorder is selected from hypercholesterolemia, hyperlipidemia, hypertriglyceridemia, sitosterolemia, atherosclerosis, arteriosclerosis, coronary heart disease, peripheral vascular disease, peripheral arterial disease, vascular inflammation, elevated Lp(a), elevated LDL, elevated TRL, elevated triglycerides, sepsis, and xanthoma.

38. A method of (i) reducing Lp(a), (ii) reducing Lp(a) plasma levels, (iii) reducing Lp(a) serum levels, (iv) reducing serum TRL or LDL levels, (v) reducing serum triglyceride levels, (vi) reducing LDL-C, (vii) reducing total plasma apoB concentrations, (viii) reducing LDL apoB, (ix) reducing TRL apoB, or (x) reducing non HDL-C, the method comprising administering to a patient in need thereof a therapeutically effective amount of a compound of claim 1 or a pharmaceutically acceptable salt thereof to the patient, thereby reducing LDL-C in the patient.

39. The method of claim 36, wherein administering is performed orally, parentally, subcutaneously, by injection, or by infusion.

40-50. (canceled)
